(19) United States
(12) Patent Application Publication
(10) Pub. No.: US 2023/0116602 A1
(43) Pub. Date: Apr. 13, 2023

(54) MCL1 INHIBITORS AND USES THEREOF

(71) Applicants: California Institute of Technology, Pasadena, CA (US); 1200 Pharma LLC, Culver City, CA (US); The Regents of the University of California, Oakland, CA (US)

(72) Inventors: Brendan M. O' Boyle, Monrovia, CA (US); Emma L. Baker-Tripp, Los Angeles, CA (US); Corey M. Reeves, San Mateo, CA (US); Kevin C. Yang, San Gabriel, CA (US); Tristin E. Rose, Los Angeles, CA (US); Justin A. Hilf, Los Angeles, CA (US); Brian M. Stoltz, San Marino, CA (US); Michael D. Bartberger, Sherman Oaks, CA (US); Oliver C. Loson, Monrovia, CA (US); Martina S. McDermott, Los Angeles, CA (US); Neil A. O'Brien, Los Angeles, CA (US); Dennis Slamon, Woodland Hills, CA (US)

(21) Appl. No.: 17/766,147

(22) PCT Filed: Oct. 2, 2020
(86) PCT No.: PCT/US20/54095

§ 371 Date: Apr. 1, 2022

Related U.S. Application Data
(60) Provisional application No. 63/065,755, filed on Aug. 14, 2020.
(60) Provisional application No. 62/964,964, filed on Jan. 23, 2020.
(60) Provisional application No. 62/910,146, filed on Oct. 3, 2019.

(51) Int. Cl. C07D 513/04; C07D 417/14; C07D 471/04; A61P 35/00

(52) U.S. Cl. CPC C07D 513/04; C07D 417/14; C07D 471/04; A61P 35/00

(57) ABSTRACT

The present disclosure provides compounds, such as compounds of Formula I, and compositions that are MCL1 inhibitors.

### CROSS-REFERENCE TO RELATED APPLICATIONS

This application claims the benefit of U.S. Provisional Application 63/065,755, filed Aug. 14, 2020, U.S. Provisional Application No. 62/964,964, filed Janaury 23, 2020, and U.S. Provisional Application No. 62/910,146, filed Oct. 3, 2019, each of which is incorporated by reference herein in its entirety.

### BACKGROUND

MCL1 (also abbreviated as MCl-1, MCL-1, Mcl1 or Mcl-1) protein is a member of the BCL2 family of proteins. The BCL2 family regulates apoptosis. Members of the BCL2 family include the pro-apoptotic proteins BAX and BAK, which, when activated, translocate to the outer membrane of mitochondria, where they form homo-oligomers. These oligomers cause pore formation in the outer mitochondrial membrane and triggers apoptosis. Other members of the BCL2 family, including BCL2, BCLXL and MCL1, prevent apoptosis (i.e., they are anti-apoptotic).

The pathological mechanisms of certain diseases are known to involve the deregulation of apoptosis. For example, increased apoptosis is implicated in the neurodegenerative diseases Parkinson's disease, Alzheimer's disease and ischemia. In contrast, deficiencies in apoptosis are implicated in the development of cancers and their chemoresistances, in auto-immune diseases, inflammatory diseases and viral infections.

The anti-apoptotic proteins of the BCL2 family are associated with several cancers, such as, for example, colon cancer, breast cancer, non-small-cell lung cancer, small-cell lung cancer, bladder cancer, prostate cancer, lymphoma, myeloma, acute myeloid leukemia (also called acute myelogenous leukemia), chronic lymphocytic leukemia, pancreatic cancer, and ovarian cancer.

Some cancers overexpress MCL1. This overexpression prevents cancer cells from undergoing apoptosis, which allows them to survive and leads to disease progression. It is understood in the art that MCL1 inhibitors can be useful for the treatment of cancers.

Therefore, a welcomed contribution to the art would be small-molecules (i.e., compounds) that inhibit MCL1 activity for treating a broad spectrum of cancers, such as, for example, myeloma, lymphoma, acute myelogenous leukemia, melanoma, sarcoma, pancreatic cancer, thyroid cancer, colorectal cancer, lung cancer, breast cancer, and ovarian cancer.

### SUMMARY

In certain embodiments, the invention relates to a compound having:

(a) the structure of Formula I:

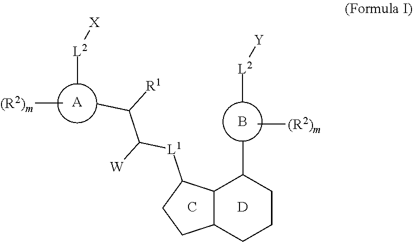

or a pharmaceutically acceptable salt thereof,
wherein:

A is aryl or heteroaryl;

B is a bond, aryl or heteroaryl;

the 5,6-membered bicyclic heteroaryl represented by C and D is selected from:

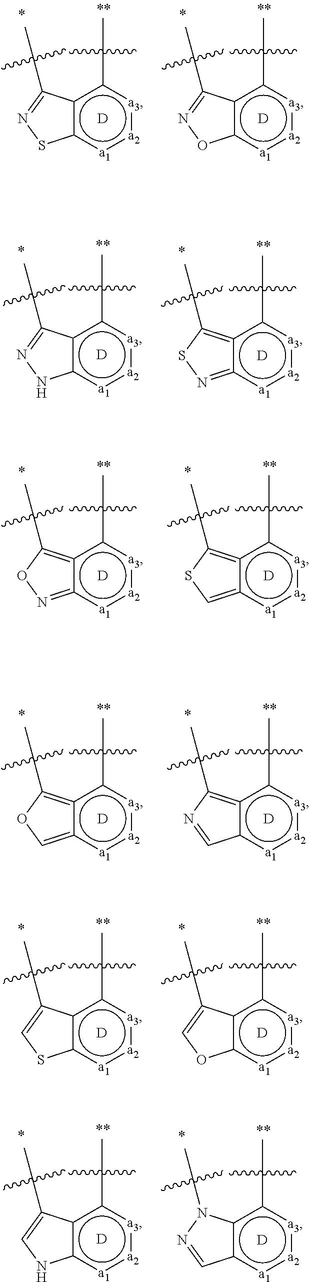

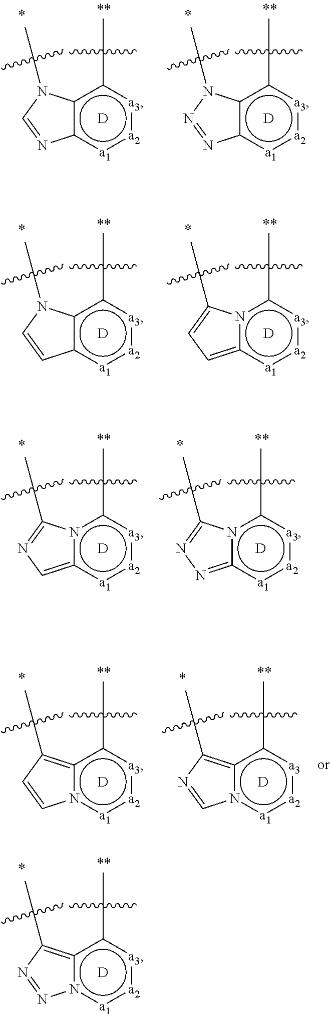

where [symbol] represents the points of attachment, * represents the point attaching to L¹, and ** represents the point attaching to B;
a₁ is CH, N or NH;
a₂ is C(Z¹), N or N(Z²);

a₃ is C(Z¹), N or N(Z²),

provided that a₁, a₂, and as are selected such that ring D is aromatic;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

W is C(O)OR^{W1}, C(O)N(H)S(O)₂R^{W2}, S(O)₂N(H)C(O)R^{W2}, S(O)₂N(H)R^{W3},

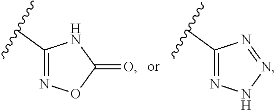 [SMILES: *c1noc(=O)[nH]1 ; *c1nn[nH]n1]

where [symbol] represents the point of attachment;

X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X2}), or hydroxyl, wherein each of said aryl, heteroaryl, cycloalkyl and heterocyclyl is optionally substituted with one, two, three or four R^{X3};

Z¹ is H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Z² is H, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

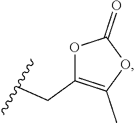 [SMILES: *Cc1oc(=O)oc1C]

where [symbol] represents the point of attachment, wherein each of said heterocyclyl, aryl and heteroaryl is optionally substituted with one, two, three or four R^{W3a}.

R^{W2} is C₁-C₆ alkyl, cycloalkyl, heterocyclyl, aryl or heteroaryl;

R^{W3} is aryl or heteroaryl;

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or

R^{X1} and R^{X2}, together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4, with the proviso that n is 0 when B is a bond; and

p in each instance is independently 0 or 1; or

(b) the structure of Formula II:

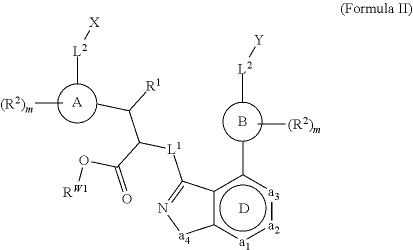

or a pharmaceutically acceptable salt thereof,
wherein:

A is aryl or heteroaryl;

B is a bond, aryl or heteroaryl;

a₁ is CH, N or NH;

a₂ is C(Z¹), N or N(Z²);

a₃ is C(Z¹), N or N(Z²);

a₄ is S, O or NH,

provided that a₁, a₂, and as are selected such that ring D is aromatic;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X2}), or hydroxyl, wherein each of said aryl, heteroaryl, cycloalkyl and heterocyclyl is optionally substituted with one, two, three or four R^{X3};

Z¹ is H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Z² is H, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

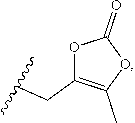 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or

R^{X1} and R^{X2}, together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4, with the proviso that n is 0 when B is a bond; and

p in each instance is independently 0 or 1; or

(c) the structure of Formula III:

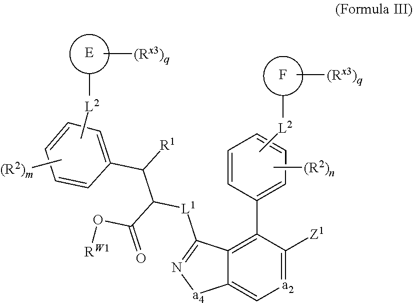

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is absent, aryl, heteroaryl, cycloalkyl or heterocyclyl;

F is aryl, heteroaryl, cycloalkyl or heterocyclyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

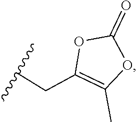 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3}a is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3}.

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1; and

q in each instance is independently 0, 1, 2, 3 or 4, with the proviso that q is 0 in —(R^{X3})_{q} when E is absent;

(d) the structure of Formula IV:

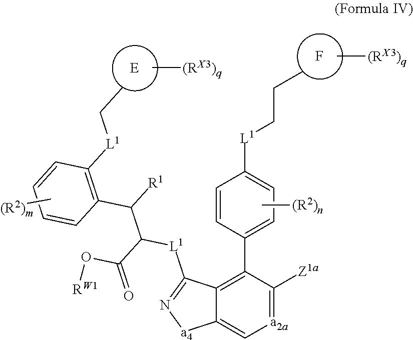

or a pharmaceutically acceptable salt thereof,
wherein:

a₂a is CH or N;

a₄ is S, O or NH;

L¹ in each instance is independently a bond, CH₂, O, NH, S, SO, or SO₂;

E is absent, aryl, heteroaryl, cycloalkyl or heterocyclyl;

F is aryl, heteroaryl, cycloalkyl or heterocyclyl;

Z^{1a} is H, halogen, aryl, heteroaryl, cycloalkyl or heterocyclyl, wherein each of said aryl, heteroaryl, cycloalkyl and heterocyclyl is optionally substituted with one or more R^{Z1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

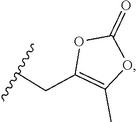 [SMILES: *Cc1oc(=O)oc1C]

where [symbol] represents the point of attachment, wherein each of said heterocyclyl, aryl and heteroaryl is optionally substituted with one, two, three or four R^{W3a}.

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

m is 0, 1, 2, or 3;

n is 0, 1, 2, 3 or 4; and

q in each instance is independently 0, 1, 2, 3 or 4, with the proviso that q is 0 in —(R^{X3})_{q} when E is absent; or

(e) the structure of Formula V:

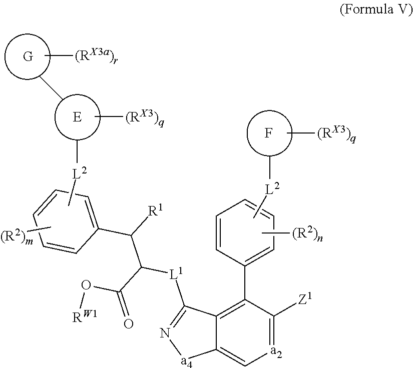

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is aryl, heteroaryl, cycloalkyl or heterocyclyl;

F is aryl, heteroaryl, cycloalkyl or heterocyclyl;

G is aryl, heteroaryl, cycloalkyl or heterocyclyl; Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

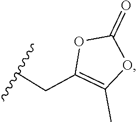 [SMILES: *Cc1oc(=O)oc1C]

where [symbol] represents the point of attachment, wherein each of said heterocyclyl, aryl and heteroaryl is optionally substituted with one, two, three or four R^{W3a}.

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}) amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen; R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1;

q in each instance is independently 0, 1, 2, 3 or 4; and

r is 0, 1, 2, 3 or 4; or

(f) the structure of Formula VI:

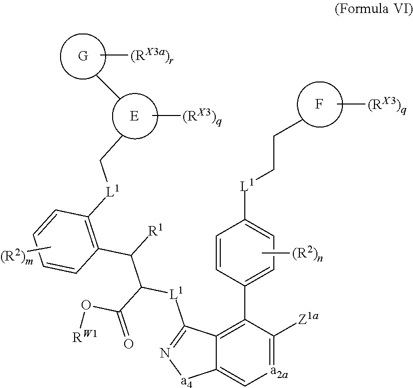

or a pharmaceutically acceptable salt thereof,
wherein:

a₂ is C(Z¹) or N;

a₄ is S, O or NH;

L¹ in each instance is independently a bond, CH₂, O, NH, S, SO, or SO₂;

E is aryl, heteroaryl, cycloalkyl or heterocyclyl;

F is aryl, heteroaryl, cycloalkyl or heterocyclyl;

G is aryl, heteroaryl, cycloalkyl or heterocyclyl; Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

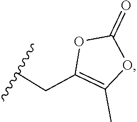 [SMILES: *Cc1oc(=O)oc1C]

where [symbol] represents the point of attachment, wherein each of said heterocyclyl, aryl and heteroaryl is optionally substituted with one, two, three or four R^{W3a}.

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}) amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen; R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

q in each instance is independently 0, 1, 2, 3 or 4; and

r is 0, 1, 2, 3 or 4;

(g) the structure of Formula VII:

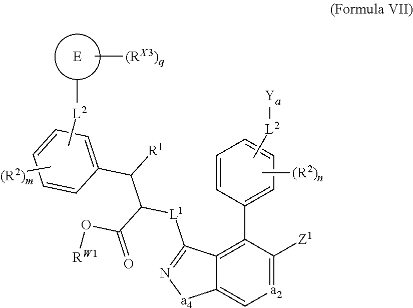

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is absent, aryl, heteroaryl, cycloalkyl or heterocyclyl;

Yₐ is C₁-C₆ hydroxyalkyl, C₁-C₆ alkoxy, N(R^{X1})(R^{X2}), or hydroxyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

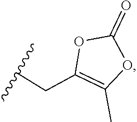 [SMILES: *Cc1oc(=O)oc1C]

where [symbol] represents the point of attachment, wherein each of said heterocyclyl, aryl and heteroaryl is optionally substituted with one, two, three or four R^{W3a}.

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}) amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen; R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1; and

q in each instance is independently 0, 1, 2, 3 or 4, with the proviso that q is 0 in —(R^{X3})_{q} when E is absent;

(h) the structure of Formula VIII:

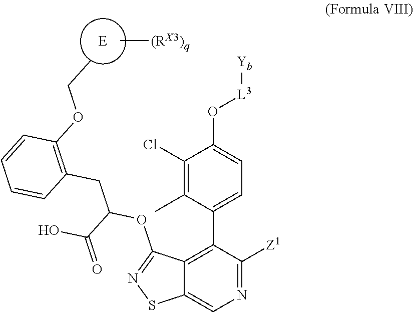

or a pharmaceutically acceptable salt thereof,
wherein:

E is heteroaryl;

L³ is —CH₂—, or —CH₂CH₂—;

Y_{b} is H, heterocyclyl, —N(CH₃)₂, —N(CH₂CH₃)₂, —CH₂N(CH₃)₂ or —CH₂N(CH₂CH₃)₂; or

L³ is absent and Y_{b} is H;

Z¹ is aryl, heteroaryl, cycloalkyl or heterocyclyl, wherein each of said aryl, heteroaryl, cycloalkyl and heterocyclyl is optionally substituted with one or more R^{Z1};

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, heterocyclyl and C₁-C₆ alkoxy is optionally substituted with one, two, three or four R^{X3}a;

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano; and

q is 0, 1, 2, 3 or 4; or

(i) the structure of Formula IX:

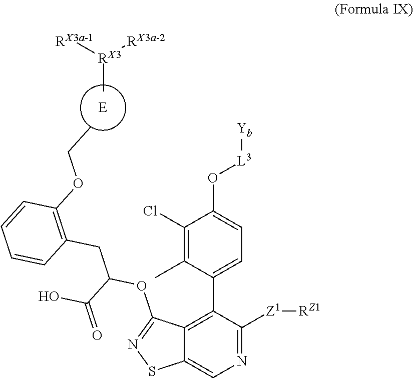

or a pharmaceutically acceptable salt thereof,
wherein:

E is pyrimidinyl, pyrazolyl, pyridinyl or imidazolyl;

L³ is —CH₂— or —CH₂CH₂—;

Y_{b} morpholinyl, piperazinyl, piperidinyl, N(CH₂CH₃)₂ or N(CH₃)₂;

Z¹ is cyclobutyl, benzyl, pyridinyl, pyrazolyl or imidazolyl;

R^{X3} benzyl, pyridinyl, C₁-C₃ alkoxy or C₁-C₄ alkyl;

R^{X3a-1} and R^{X3a-2} is each independently H, C₁-C₃ alkyl, C₁-C₂ haloalkyl, amino, cyano or Halogen; and

R^{Z1} is C₁-C₃ alkyl, C₁-C₃ haloalkyl, C₁-C₃ alkoxy or halogen.

### BRIEF DESCRIPTION OF THE DRAWINGS

FIG. 1 are the results of an AMO-1 myeloma cell line xenograft study with a compound of Formula X. Eight mice were used per group, and mice were dosed by intravenous injection (IV) daily (QD) for the first 5 days of the study with various concentrations of the compound of Formula X.

FIG. 2 shows the change in tumor volume results of the AMO-1 myeloma cell line xenograft study with the compound of Formula X.

FIG. 3 is the tabulation of the percentage weight changes per day of mice in the AMO-1 myeloma cell line xenograft study with the compound of Formula X.

### DETAILED DESCRIPTION

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, immunology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., Motulsky, “Intuitive Biostatistics”, Oxford University Press, Inc. (1995); Lodish et al., “Molecular Cell Biology, 4th ed.”, W. H.

Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The term “acyl” is art-recognized and refers to a group represented by the general formula hydrocarbylC(O)—, preferably alkylC(O)—.

An “alkyl” group or “alkane” is a straight chained or branched non-aromatic hydrocarbon which is completely saturated. Typically, a straight chained or branched alkyl group has from 1 to about 10 carbon atoms, preferably from 1 to about 6 unless otherwise defined. Examples of straight chained and branched alkyl groups include, but are not limited to methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group. Alternatively, when an alkyl group is between or conjugating two groups, it is considered an alkylene.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, an oxo, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁-C₆ alkyl, C₃-C₆ cycloalkyl, halogen, amino, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “alkenyl,” as used herein, refers to an aliphatic group containing at least one double bond and is intended to include both “unsubstituted alkenyls” and “substituted alkenyls” the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkynyl,” as used herein, refers to an aliphatic group containing at least one triple bond and is intended to include both “unsubstituted alkynyls” and “substituted alkynyls,” the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “alkylamino,” as used herein, refers to an amino group substituted with at least one alkyl group.

The term “alkylthio,” as used herein, refers to a thiol group substituted with an alkyl group and may be represented by the general formula alkylS—.

The term “Cₓ-C_{y},” when used in conjunction with a chemical moiety, such as, acyl, acyloxy, alkyl, alkenyl, alkynyl, or alkoxy is meant to include groups that contain from x to y carbons in the chain. For example, the term “Cₓ-C_{y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C₂-C_{y} alkenyl” and “C₂-C_{y} alkynyl” refer to substituted or unsubstituted unsaturated aliphatic groups analogous in length and possible substitution to the alkyls described above, but that contain at least one double or triple bond respectively.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The terms “amine” and “amino” are art-recognized and refer to both unsubstituted and substituted amines and salts thereof, e.g., a moiety that can be represented by

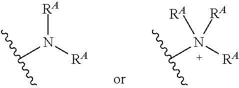

wherein each R^{A} independently represents a hydrogen or a hydrocarbyl group, or two R^{A} are taken together with the N atom to which they are attached to complete a heterocycle having from 4 to 8 atoms in the ring structure.

The term “aminoalkyl,” as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- to 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, aniline, and the like.

The term “carbocycle” refers to a saturated or unsaturated ring in which each atom of the ring is carbon. The term carbocycle includes both aromatic carbocycles and non-aromatic carbocycles. Non-aromatic carbocycles include both cycloalkyl and cycloalkenyl rings. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3- to about 10-carbon atoms, from 3- to 8-carbon atoms, or more typically from 3- to 6-carbon atoms unless otherwise defined. The second ring of a bicyclic cycloalkyl may be selected from saturated, unsaturated and aromatic rings. Cycloalkyl includes bicyclic molecules in which one, two, or three or more atoms are shared between the two rings (e.g., fused bicyclic compounds, bridged bicyclic compounds, and spirocyclic compounds).

The term “fused bicyclic compound” refers to a bicyclic molecule in which two rings share two adjacent atoms. In other words, the rings share one covalent bond, i.e., the so-called bridgehead atoms are directly connected (e.g., α-thujene and decalin). For example, in a fused cycloalkyl each of the rings shares two adjacent atoms with the other ring, and the second ring of a fused bicyclic cycloalkyl may be selected from saturated, unsaturated and aromatic rings.

The term “spirocyclic compound” or “spirocycle” refers to a bicyclic molecule or group in which the two rings have only one single atom, the spiro atom, in common.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, quinoline, quinoxaline, naphthyridine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, preferably 3- to 7-membered rings, more preferably 5- to 6-membered rings, in some instances, most preferably a 5-membered ring, in other instances, most preferably a 6-membered ring, which ring structures include at least one heteroatom, preferably one to four heteroatoms, more preferably one or two heteroatoms. The terms “heterocyclyl” and “heterocyclic” also include polycyclic ring systems having two or more cyclic rings in which one, two or more carbons (e.g., fused heterobicyclic compounds, bridged heterobicyclic compounds, and heterospirocyclic compounds) are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. The terms “heterocyclyl” and “heterocyclic” further include spirocycles, wherein at least one of the rings is heterocyclic, e.g., the other cyclic ring can be cycloalkyl, cycloalkenyl, cycloalkynyl, and/or heterocyclyl. Heterocyclyl groups include, for example, pyrrolidine, piperidine, piperazine, pyrrolidine, tetrahydropyran, tetrahydrofuran, morpholine, lactones, lactams, oxazolines, imidazolines and the like.

The terms “halo” and “halogen” as used herein means halogen and includes chloro, fluoro, bromo, and iodo.

The term “haloalkyl,” as used herein, refers to an alkyl group substituted with one or more halo.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “sulfonamide” is art-recognized and refers to the group represented by the general formulae

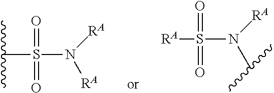

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonyl” is art-recognized and refers to the group —S(O)₂—R^{A}, wherein R^{A} represents a hydrocarbyl.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substitutions can be one or more and the same or different for appropriate organic compounds.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt that is suitable for or compatible with the treatment of patients.

The term “pharmaceutically acceptable acid addition salt” as used herein means any non-toxic organic or inorganic salt of any base compounds disclosed herein.

Illustrative inorganic acids that form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the invention for laboratory use, or for subsequent conversion to a pharmaceutically acceptable acid addition salt.

The term “pharmaceutically acceptable basic addition salt” as used herein means any non-toxic organic or inorganic base addition salt of any acid compounds of the invention, or any of their intermediates. Illustrative inorganic bases that form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixtures and separate individual isomers.

Some of the compounds may also exist in tautomeric forms. Such forms, although not explicitly indicated in the formulae described herein, are intended to be included within the scope of the present disclosure.

This disclosure contemplates all rotational isomers and atropisomers of the compounds and the salts, drugs, prodrugs, or mixtures thereof (including all possible mixtures of rotational isomers). Structures shown without stereochemistry are intended to cover one, the other, or a mixture of both rotational isomers or atropisomers.

“Prodrug” or “pharmaceutically acceptable prodrug” refers to a compound that is metabolized, for example hydrolyzed or oxidized, in the host after administration to form the compound of the present disclosure (e.g., compounds of the invention). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of the invention, or a pharmaceutically acceptable salt thereof. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

### Example Compounds

In certain embodiments, the invention relates to a compound having the structure of Formula I:

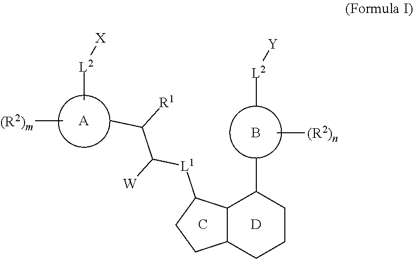

or a pharmaceutically acceptable salt thereof,
wherein.

A is aryl or heteroaryl;

B is a bond, aryl or heteroaryl;

the 5,6-membered bicyclic heteroaryl represented by C and D is selected from:

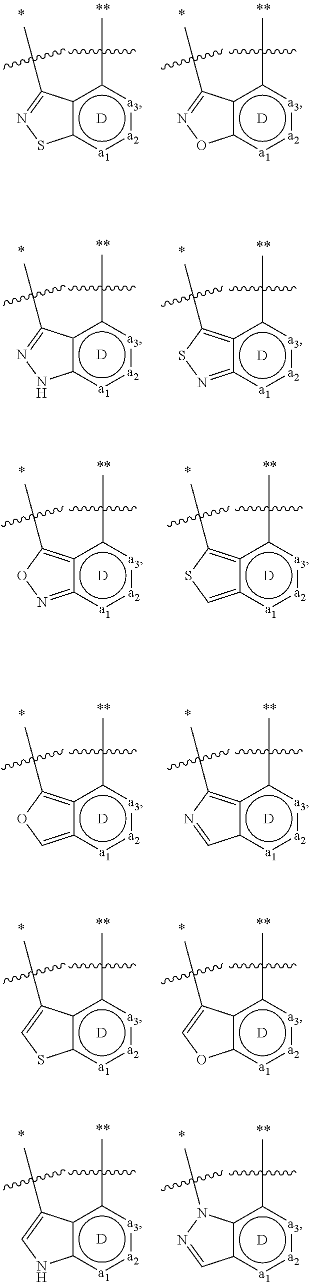

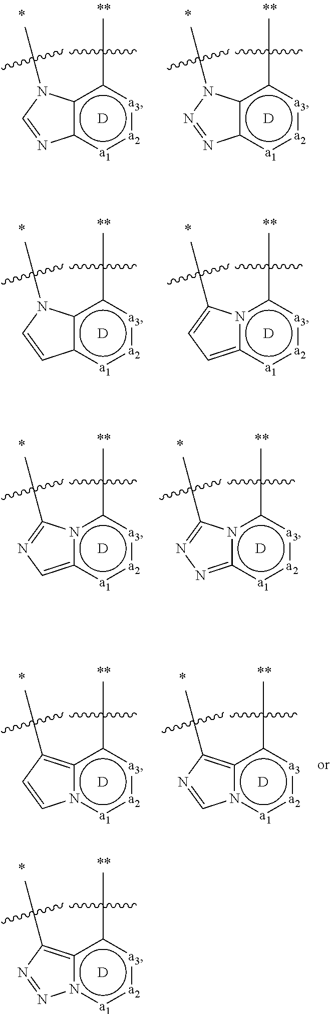

where [symbol] represents the points of attachment, * represents the point attaching to L¹, and ** represents the point attaching to B;

a₁ is CH, N, or NH;

a₂ is C(Z¹), N or N(Z²);

a₃ is C(Z¹), N or N(Z²);

provided that a₁, a₂, and a₃ are selected such ring D is aromatic;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ—N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(o)—(C₁-C₆ alkyl)ₚ—, or —(C₁-C₆ alkyl)ₚ—S(o)₂—(C₁-C₆ alkyl)ₚ—;

W is C(O)OR^{W1}, C(O)N(H)S(O)₂R^{W2}, S(O)₂N(H)C(O)R^{W2}, S(O)₂N(H)R^{W3}

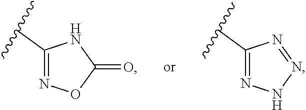 [SMILES: *c1noc(=O)[nH]1 ; *c1nn[nH]n1]

where [symbol] represents the point of attachment;

X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X}), or hydroxyl, wherein each of said aryl, heteroaryl, cycloalkyl and heterocyclyl is optionally substituted with one, two, three or four R^{X3};

Z¹ is H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Z² is H, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

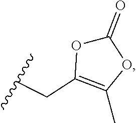 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W2} is C₁-C₆ alkyl, cycloalkyl, heterocyclyl, aryl or heteroaryl;

R^{W3} is aryl or heteroaryl;

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3}a is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or

R^{X1} and R^{X2}, together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl, and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4, with the proviso that n is 0 when B is a bond; and

p in each instance

is independently 0 or 1.

In certain embodiments, the invention relates to a compound of Formula I, wherein:

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, sulfonamide, sulfoxide, sulfonyl, or cyano, wherein each of C₁-C₆ alkyl, C₁-C₆ alkoxy and C₁-C₆ hydroxyalkyl is optionally substituted with one, two, three or four groups each independently selected from halogen, amino, nitro, sulfonamide, sulfoxide, sulfonyl or cyano.

In certain embodiments, the invention relates to a compound of Formula I,

wherein:

B is aryl or heteroaryl;

X is aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, or cyano; and

n is 0, 1, 2, 3 or 4.

In certain embodiments, the invention relates to a compound having the structure of Formula II:

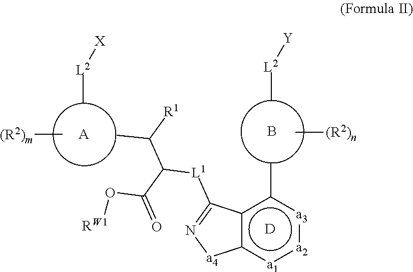

or a pharmaceutically acceptable salt thereof,

wherein:

A is aryl or heteroaryl;

B is a bond, aryl or heteroaryl;

a₁ is CH, N or NH;

a₂ is C(Z¹), N or N(Z²);

a₃ is C(Z¹), N or N(Z²);

a₄ is S, O or NH,

provided that a₁, a₂, and as are selected such that ring D is aromatic;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X}), or hydroxyl, wherein each of said aryl, heteroaryl, cycloalkyl and heterocyclyl is optionally substituted with one, two, three or four R^{X3};

Z¹ is H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Z² is H, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

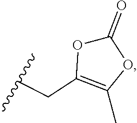 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or

R^{X1} and R^{X2}, together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen.

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4, with the proviso that n is 0 when B is a bond; and

p in each instance is independently 0 or 1.

In certain embodiments, the invention relates to a compound of Formula II, wherein:

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, sulfonamide, sulfoxide, sulfonyl, or cyano, wherein each of C₁-C₆ alkyl, C₁-C₆ alkoxy and C₁-C₆ hydroxyalkyl is optionally substituted with one, two, three or four groups each independently selected from halogen, amino, nitro, sulfonamide, sulfoxide, sulfonyl or cyano.

In certain embodiments, the invention relates to a compound of Formula II, wherein:

B is aryl or heteroaryl;

X is aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, or cyano; and

n is 0, 1, 2, 3 or 4.

In some embodiments, the invention relates to a compound of Formula II, wherein:

A is aryl;

B is aryl;

a₁ is CH;

a₂ is C(H) or N;

a₃ is C(Z¹);

a₄ is S, O or NH;

L¹ is a bond, CH₂, O, NH, S, SO, or SO₂;

L² in each instance is independently a bond or —(C₁-C₆ alkyl)ₚ—O—(C₁-C₆ alkyl)ₚ—;

X is aryl or heteroaryl, wherein each of said aryl and heteroaryl is optionally substituted with one, two, three or four R^{X3};

Y is heterocyclyl, C₁-C₆ hydroxyalkyl, C₁-C₆ alkoxy, N(R^{X1})(R^{X2}), or hydroxyl, wherein heterocyclyl is optionally substituted with one, two, three or four R^{X3};

Z¹ is aryl, heteroaryl, cycloalkyl or heterocyclyl, wherein each of said aryl, heteroaryl, cycloalkyl and heterocyclyl is optionally substituted with one or more R^{Z1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R² are in each instance each independently H, C₁-C₆ alkyl, C₁-C₆ alkoxy, C₁-C₆ haloalkyl or C₁-C₆ hydroxyalkyl; or

R^{X1} and R², together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};

R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};

R^{Z3} in each instance is independently aryl, heteroaryl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

m is 0, 1, 2, or 3;

n is 0, 1, 2, 3 or 4; and

p in each instance is independently 0 or 1.

In some embodiments, the invention relates to a compound of Formula II, wherein:

A is aryl;

B is aryl;

a₁ is CH;

a₂ is C(Z¹);

a₃ is C(H);

a₄ is S, O or NH;

L¹ is a bond, CH₂, O, NH, S, SO, or SO₂;

L² in each instance is independently a bond or —(C₁-C₆ alkyl)ₚ—O—(C₁-C₆ alkyl)ₚ—;

X is aryl or heteroaryl, wherein each of said aryl and heteroaryl is optionally substituted with one, two, three or four R^{X3};

Y is heterocyclyl, C₁-C₆ hydroxyalkyl, C₁-C₆ alkoxy, N(R^{X1})(R^{X2}), or hydroxyl, wherein heterocyclyl is optionally substituted with one, two, three or four R^{X3};

Z¹ is aryl, heteroaryl, cycloalkyl or heterocyclyl, wherein each of said aryl, heteroaryl, cycloalkyl and heterocyclyl is optionally substituted with one or more R^{Z1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or, when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X1} and R^{X2} are in each instance each independently H, C₁-C₆ alkyl, C₁-C₆ alkoxy, C₁-C₆ haloalkyl or C₁-C₆ hydroxyalkyl; or

R^{X1} and R^{X2}, together with the N to which they are connected, form a heterocycle or heteroaryl, wherein each of said heterocycle and heteroaryl is optionally substituted with one, two, three or four R^{X3};

R^{X3} in each instance is independently aryl, heteroaryl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};

R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};

R^{Z3} in each instance is independently aryl, heteroaryl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

m is 0, 1, 2, or 3;

n is 0, 1, 2, 3 or 4; and

p in each instance is independently 0 or 1.

In preferred embodiments, Z¹ is a bicyclic aryl or bicyclic heteroaryl. In more preferred embodiments, the bicyclic heteroaryl is indazolyl, benzoimidazolyl, benzoimidazolonyl, indolyl, pyrrolopyridinyl or isoquinolinyl, wherein each of indazolyl, benzoimidazolyl, benzoimidazolonyl, indolyl, pyrrolopyridinyl and isoquinolinyl is optionally substituted with one, two or three groups independently selected from C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano.

In some embodiments, the invention relates to a compound of Formula I or II, wherein A is phenyl, pyridine, pyridazine, pyrimidine, pyrazine or thiophene.

In some embodiments, the invention relates to a compound of Formula I or II, wherein B is phenyl, pyridine or thiophene.

In some embodiments, the invention relates to a compound of any of Formulas I or II, wherein:

A is phenyl, pyridine or pyrimidine;

B is phenyl, pyridine or thiophene;

L¹ is O;

R¹ is H;

R² in each instance is independently C₁-C₆ alkyl or halogen;

m is 0; and

n is 2.

In some embodiments, the invention relates to a compound of Formula I or II, wherein:

Y is

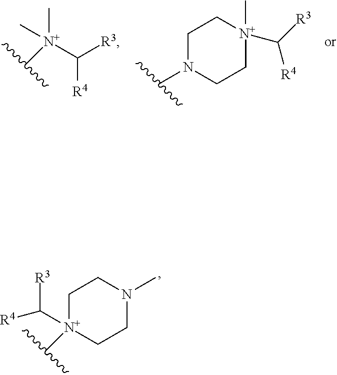

where [symbol] represents the point of attachment;

R³ is H or C₁-C₆ alkyl;

R⁴ is —O—P(O)(O⁻)(O⁻), —O—P(O)(O⁻)(OR⁵), —O—P(O)(OR⁵)(OR⁵), —O—S(O₂)—O⁻, —O—S(O₂)—OR⁵, Cy^{a}, —O—C(O)—R⁶, —O—C(O)—OR⁶, or —O—C(O)—N(R⁶)(R⁶);

Cy^{a} is cycloalkyl, heterocyclyl, aryl or heteroaryl;

R⁵ in each instance is independently H, C₁-C₆ alkyl, or aralkyl(C₁-C₆); and

R⁶ in each instance is independently H, C₁-C₆ alkyl, or C₁-C₆ aminoalkyl.

In certain embodiments, the invention relates to a compound of Formula I or II, wherein L² is a bond and Y is hydroxyl.

In certain embodiments, the invention relates to a compound having the structure of Formula III:

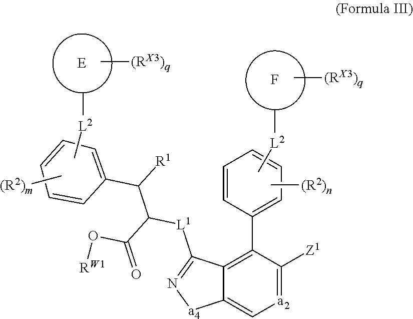

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is absent, aryl, heteroaryl, cycloalkyl or heterocyclyl;

F is aryl, heteroaryl, cycloalkyl or heterocyclyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1}; Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

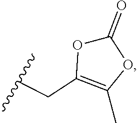 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1; and

q in each instance is independently 0, 1, 2, 3 or 4, with the proviso that q is 0 in —(R^{X3})_{q} when E is absent.

In certain embodiments, the invention relates to a compound of Formula III, wherein:

E is aryl, heteroaryl, cycloalkyl or heterocyclyl; and

q in each instance is independently 0, 1, 2, 3 or 4.

In some embodiments, the invention relates to a compound of Formula III, wherein:

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano. In some embodiments, the invention relates to a compound of Formula III, wherein:

a₂ is C(H) or N;

a₄ is S, O or NH;

L¹ is a bond, CH₂, O, NH, S, SO, or SO₂;

L² in each instance is independently a bond or —(C₁-C₆ alkyl)ₚ—O—(C₁-C₆ alkyl)ₚ—;

E is aryl or heteroaryl;

F is cycloalkyl or heterocyclyl;

Z¹ is aryl, heteroaryl, cycloalkyl or heterocyclyl, wherein each of said aryl, heteroaryl, cycloalkyl and heterocyclyl is optionally substituted with one or more R^{Z1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};

R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

m is 0, 1, 2 or 3;

n is 0, 1, 2, 3 or 4;

p in each instance is independently 0 or 1; and

q in each instance is independently 0, 1, 2, 3 or 4.

In some embodiments, the invention relates to a compound of any of Formulas I, II or III, wherein:

L¹ is O;

R¹ is H;

R² in each instance is independently C₁-C₆ alkyl or halogen;

m is 0; and

n is 2.

In other embodiments, the invention relates to a compound of any of Formula I, II or III, wherein:

R^{W1} is H;

L¹ is O;

R¹ is H;

R² in each instance is independently C₁-C₆ alkyl or halogen;

m is O; and

n is 2.

In other embodiments, the invention relates to a compound of any of Formula I, II or III, wherein a₂ is C(H) or N.

In some embodiments, the invention relates to a compound of Formula III, wherein Z¹ in each instance is independently H or halogen, and more particularly, the halogen is Br or Cl.

In other embodiments, the invention relates to a compound of Formula III, wherein Z¹ in each instance is independently H, optionally substituted phenyl, optionally substituted pyridine, optionally substituted thiophene, optionally substituted furan, optionally substituted pyrrole, optionally substituted cyclopropyl, or optionally substituted cyclobutyl. In preferred embodiments, Z¹ is cycloalkyl, and more preferably, cyclobutyl. In some embodiments, the optional substitution is C₁-C₆ alkyl, and more particularly, the C₁-C₆ alkyl is methyl or ethyl. In some embodiments, the optional substitution is halogen, and more particularly, the halogen is F or Cl.

In certain embodiments, the invention relates to a compound having the structure of Formula IV:

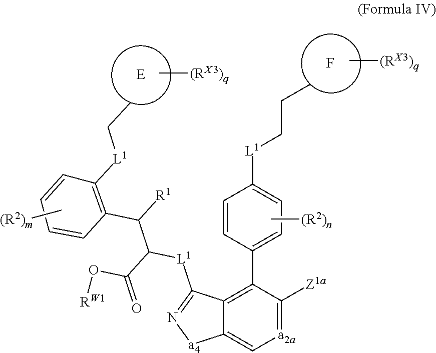

or a pharmaceutically acceptable salt thereof,
wherein:

a₂a is CH or N;

a₄ is S, O or NH;

L¹ in each instance is independently a bond, CH₂, O, NH, S, SO, or SO₂;

E is absent, aryl, heteroaryl, cycloalkyl or heterocyclyl;

F is aryl, heteroaryl, cycloalkyl or heterocyclyl;

Z^{1a} is H, halogen, aryl, heteroaryl, cycloalkyl or heterocyclyl, wherein each of said aryl, heteroaryl, cycloalkyl and heterocyclyl is optionally substituted with one or more R^{Z1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

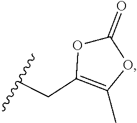 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

m is 0, 1, 2, or 3;

n is 0, 1, 2, 3 or 4; and

q in each instance is independently 0, 1, 2, 3 or 4, with the proviso that q is 0 in —(R^{X3})_{q} when E is absent.

In certain embodiments, the invention relates to a compound of Formula IV, wherein:

E is aryl, heteroaryl, cycloalkyl or heterocyclyl; and

q in each instance is independently 0, 1, 2, 3 or 4.

In some embodiments, the invention relates to a compound of Formula IV, wherein:

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3}a;

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano.

In some embodiments, the invention relates to a compound of Formula III or IV, wherein:

R¹ is H;

R² in each instance is independently C₁-C₆ alkyl or halogen;

m is 0; and

n is 2.

In some embodiments, the invention relates to a compound of Formula IV, wherein Z^{1a} is halogen, and more particularly, the halogen is Br or Cl.

In other embodiments, the invention relates to a compound of Formula IV, wherein Z^{1a} is optionally substituted phenyl, optionally substituted pyridine, optionally substituted thiophene, optionally substituted furan, optionally substituted pyrrole, optionally substituted cyclopropyl, or optionally substituted cyclobutyl. In some embodiments, the optional substitution is C₁-C₆ alkyl, and more particularly, the C₁-C₆ alkyl is methyl or ethyl. In some embodiments, the optional substitution is halogen, and more particularly, the halogen is F or Cl.

In some embodiments, the invention relates to a compound of Formula III or IV, wherein:

E is phenyl, pyridine, pyridazine, pyrimidine, pyrazine, triazine, furan, thiophene, pyrrole, pyrazole, imidazole or triazole;

F is pyrrolidine, piperidine, piperazine, tetrahydropyran, morpholine, 2,6-diazaspiro[3.3]heptanyl, 2-oxa-6-azaspiro[3.3]heptanyl, 2-azaspiro[3.3]heptanyl, or 2-oxaspiro[3.3]heptanyl;

R^{X3} in each instance is independently C₁-C₆ alkyl, C₁-C₆ alkoxy, C₁-C₆ haloalkyl, C₁-C₆ hydroxyalkyl, hydroxy, halogen, amino, nitro or cyano; and

q in each instance is independently 0, 1 or 2.

In certain embodiments, the invention relates to a compound having the structure of Formula V:

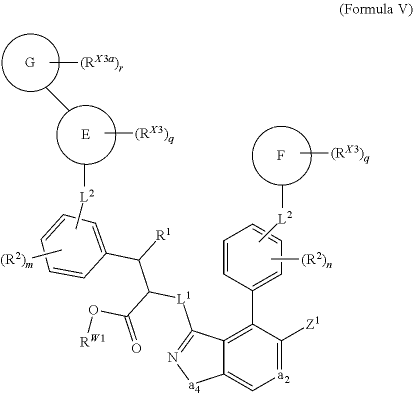

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R¹)—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is aryl, heteroaryl, cycloalkyl or heterocyclyl;

F is aryl, heteroaryl, cycloalkyl or heterocyclyl;

G is aryl, heteroaryl, cycloalkyl or heterocyclyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

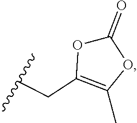 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a}.

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen; R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1;

q in each instance is independently 0, 1, 2, 3 or 4; and

r is 0, 1, 2, 3 or 4.

In certain embodiments, the invention relates to a compound having the structure of Formula V, wherein:

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, sulfonamide, sulfoxide, sulfonyl or cyano.

In certain embodiments, the invention relates to a compound having the structure of Formula VI:

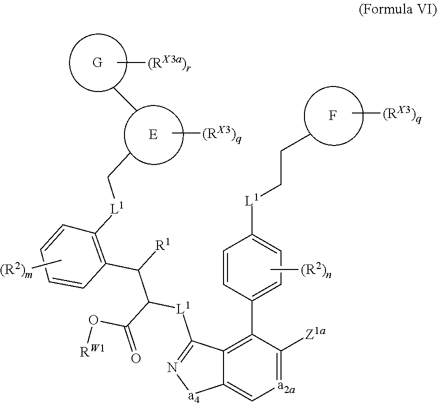

or a pharmaceutically acceptable salt thereof,
wherein:

a₂ is C(Z¹) or N;

a₄ is S, O or NH;

L¹ in each instance is independently a bond, CH₂, O, NH, S, SO, or SO₂;

E is aryl, heteroaryl, cycloalkyl or heterocyclyl;

F is aryl, heteroaryl, cycloalkyl or heterocyclyl;

G is aryl, heteroaryl, cycloalkyl or heterocyclyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

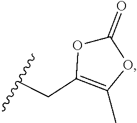 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};

R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};

R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;

R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or

R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen; R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

q in each instance is independently 0, 1, 2, 3 or 4; and

r is 0, 1, 2, 3 or 4.

In some embodiments, the invention relates to a compound of Formula VI, wherein

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro, sulfonamide, sulfoxide, sulfonyl or cyano.

In some embodiments, the invention relates to a compound of Formula III, IV, V or VI, wherein:

F is

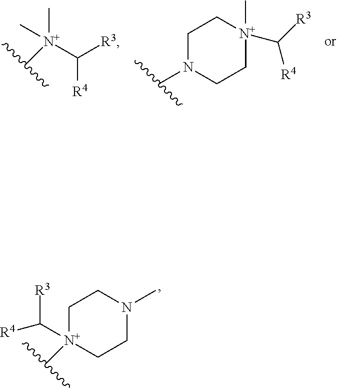

where [symbol] represents the point of attachment;

R³ is H or C₁-C₆ alkyl;

R⁴ is —O—P(O)(O⁻)(O⁻), —O—P(O)(O⁻)(OR⁵), —O—P(O)(OR⁵)(OR⁵), —O—S(O₂)—O⁻, —O—S(O₂)—OR⁵, Cy^{a}, —O—C(O)—R⁶, —O—C(O)—OR⁶, or —O—C(O)—N(R⁶)(R⁶);

R⁵ in each instance is independently H, C₁-C₆ alkyl, or aralkyl(C₁-C₆);

R⁶ in each instance is independently H, C₁-C₆ alkyl, or C₁-C₆ aminoalkyl; and

q, in the instance of F substitution with R^{X3}, is 0.

In certain embodiments, the invention relates to a compound having the structure of Formula VII:

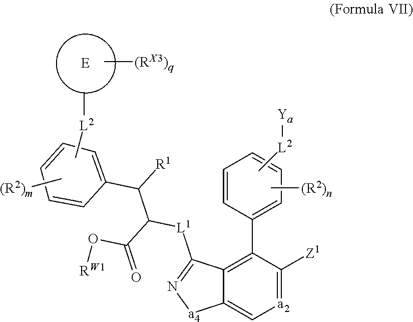

or a pharmaceutically acceptable salt thereof,
wherein:

a₂ is C(Z¹) or N;

a₄ is S, O or NH;

L¹ is a bond, CH₂, O, NH, S, SO, or SO₂;

L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R¹)—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;

E is absent, aryl, heteroaryl, cycloalkyl or heterocyclyl;

Yₐ is C₁-C₆ hydroxyalkyl, C₁-C₆ alkoxy, N(R^{X1})(R^{X2}), or hydroxyl;

Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};

Cy is aryl, heteroaryl, cycloalkyl or heterocyclyl, wherein each is optionally substituted with one, two, three or four R^{Cy1};

R¹ is H, hydroxy, C₁-C₃ alkyl, C₁-C₂ haloalkyl, C₁-C₂ hydroxyalkyl, or C₁-C₂ alkoxy;

R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});

R^{2a} and R^{2b} are each independently H, C₁-C₆ alkyl, C₁-C₆ haloalkyl, or C₁-C₆ hydroxyalkyl;

R^{W1} is H, C₁-C₆ alkyl, CH(R^{W1a})(R^{W2a}), heterocyclyl, aryl, heteroaryl, or

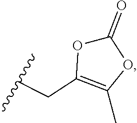 [SMILES: *Cc1oc(=O)oc1C]

where [symbol] represents the point of attachment, wherein each of said heterocyclyl, aryl and heteroaryl is optionally substituted with one, two, three or four R^{W3a};

R^{W1a} is H or C₁-C₆ alkyl;

R^{W2a} is OC(O)OR^{W2b}, OC(O)N(R^{W2b})(R^{W2b}) or OP(O)(OR^{W2b})₂;

R^{W2b} in each instance is independently H, C₁-C₆ alkyl, cycloalkyl or C₁-C₆ alkoxy; or,

when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};

R^{W3a} is C₁-C₆ alkyl, C₁-C₆ alkoxy, C₁-C₆ haloalkyl, C₁-C₆ hydroxyalkyl, hydroxy, halogen, amino, nitro or cyano;

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, and heterocyclyl is optionally substituted with one, two, three or four R^{X3a};

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;

R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;

R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;

m is 0, 1, 2, or 3;

n is 0, 1, 2, 3 or 4;

p in each instance is independently 0 or 1; and

q in each instance is independently 0, 1, 2, 3 or 4, with the proviso that q is 0 in —(R^{X3})_{q} when E is absent.

In some embodiments, the invention relates to a compound of Formula VII, wherein:

E is phenyl, pyridine, pyridazine, pyrimidine, pyrazine, triazine, furan, thiophene, pyrrole, pyrazole, imidazole or triazole;

Yₐ is N(R^{X1})(R^{X2}); and

q in each instance is independently 0, 1 or 2.

In some embodiments, the invention relates to any compound described herein, wherein C₁-C₆ haloalkyl is trifluoromethane or trifluoroethane.

In other embodiments, the invention relates to a compound of Formula I, II, III, IV, V, VI or VII, wherein R^{W1} is

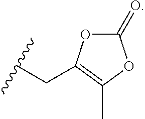 [SMILES: *Cc1oc(=O)oc1C]

In certain embodiments, the invention relates to a compound having the structure of Formula VIII:

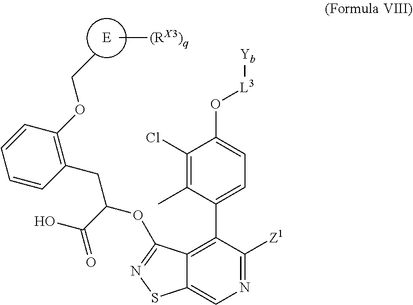

or a pharmaceutically acceptable salt thereof,
wherein:

E is heteroaryl;

L³ is —CH₂—, or —CH₂CH₂—;

Y_{b} is H, heterocyclyl, —N(CH₃)₂, —N(CH₂CH₃)₂, —CH₂N(CH₃)₂ or —CH₂N(CH₂CH₃)₂; or

L³ is absent and Y_{b} is H;

Z¹ is aryl, heteroaryl, cycloalkyl or heterocyclyl, wherein each of said aryl, heteroaryl, cycloalkyl and heterocyclyl is optionally substituted with one or more R^{Z1};

R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, heterocyclyl and C₁-C₆ alkoxy is optionally substituted with one, two, three or four R^{X3}a;

R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;

R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};

R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or

two R^{Z2}, together with the N to which they are connected, form a heterocycle or heteroaryl, wherein each of said heterocycle and heteroaryl is optionally substituted with one, two, three or four R^{Z3};

R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};

R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano; and

q is 0, 1, 2, 3 or 4.

In some embodiments, the invention relates to a compound of Formula VIII, wherein:

E is pyrimidynyl or pyrazolyl;

L³ is —CH₂CH₂—;

Y_{b} is heterocyclyl;

Z¹ is cycloalkyl; and

q is 0, 1 or 2.

In other embodiments, the compound of Formula VIII has an MCL1 IC₅₀ of about 100 nM or lower.

In other embodiments, the compound of Formula VIII has an average IC₅₀ for the drug sensitive cell lines of Table 3 of 1 μM or lower.

In yet other embodiments, the compound of Formula VIII has an average IC₅₀ for the drug-sensitive cell lines of Table 3 that is at least about 10-fold more potent than the average IC₅₀ for the drug-resistant cell lines of Table 3.

In certain embodiments, the invention relates to a compound having the structure of Formula IX:

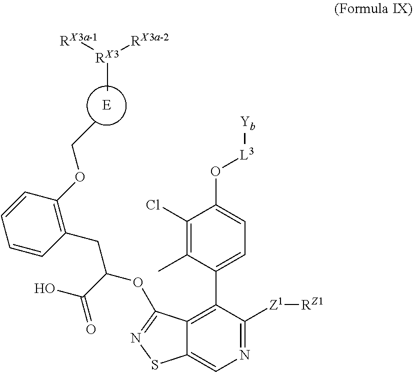

or a pharmaceutically acceptable salt thereof,
wherein:
E is pyrimidinyl, pyrazolyl, pyridinyl or imidazolyl;

L³ is —CH₂— or —CH₂CH₂—;

Y_{b} morpholinyl, piperazinyl, piperindinyl, N(CH₂CH₃)₂ or N(CH₃)₂;

Z¹ is cyclobutyl, benzyl, pyridinyl, pyrazolyl or imidazolyl;

R^{X3} benzyl, pyridinyl, C₁-C₃ alkoxy or C₁-C₄ alkyl;

R^{X3a-1} and R^{X3a-2} is each independently H, C₁-C₃ alkyl, C₁-C₂ haloalkyl, amino, cyano or Halogen; and

R^{Z1} is C₁-C₃ alkyl, C₁-C₃ haloalkyl, C₁-C₃ alkoxy or halogen.

In certain embodiments, the invention relates to a compound having the structure of Formula X:

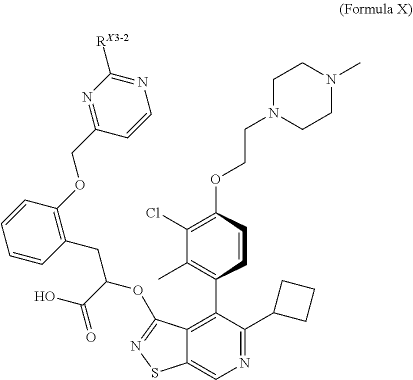

or a pharmaceutically acceptable salt thereof, wherein R^{X3-2} is

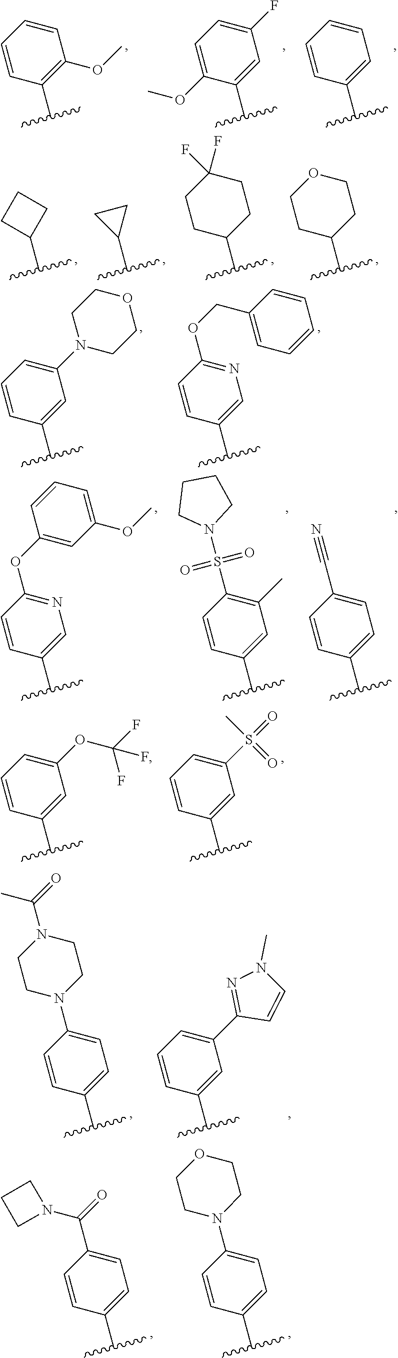

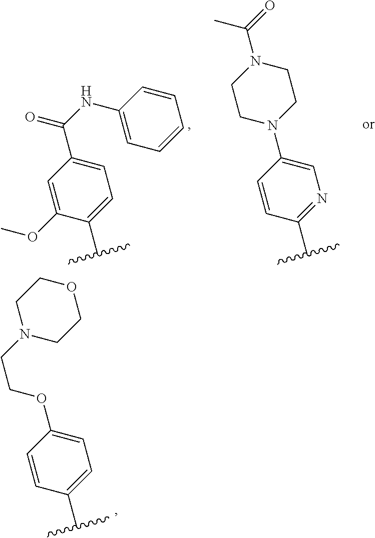

where [symbol] represents the point of attachment.

In specific embodiments, R^{X3-2} is

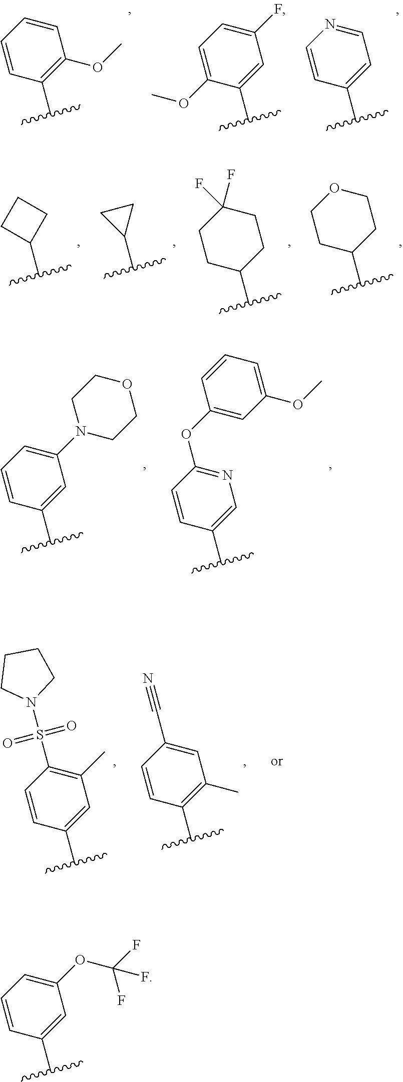

In preferable embodiments, R^{X3-2} is

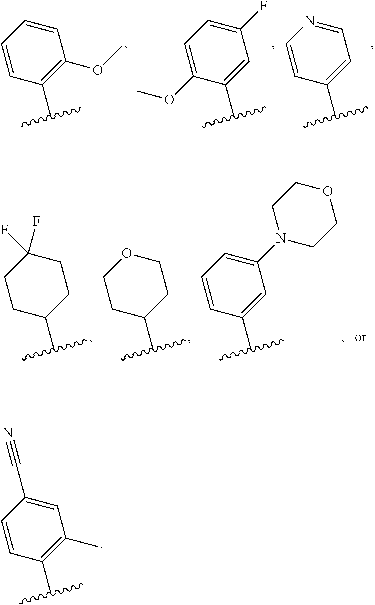

In yet other embodiments, the compound of Formula VIII is selected from:

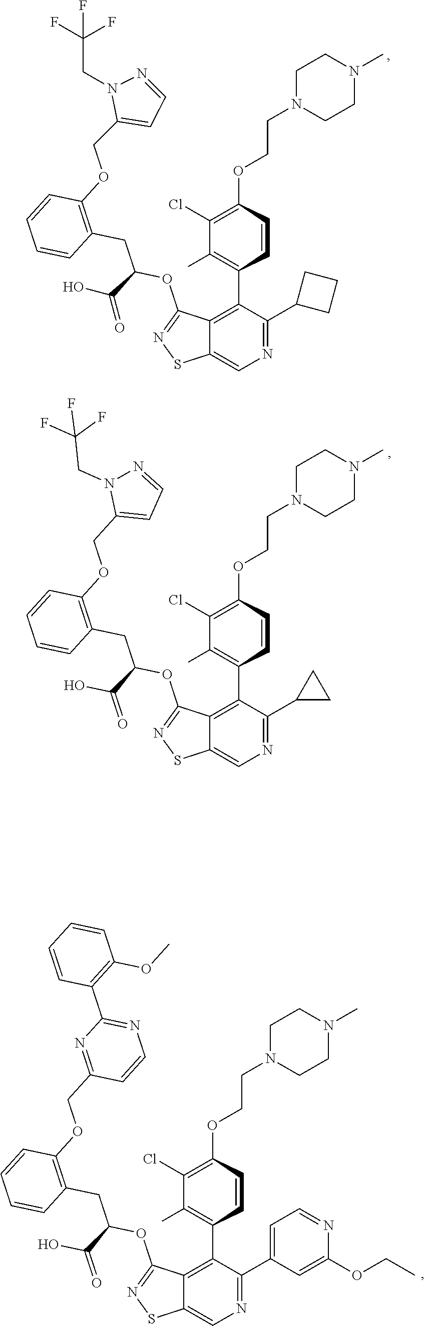

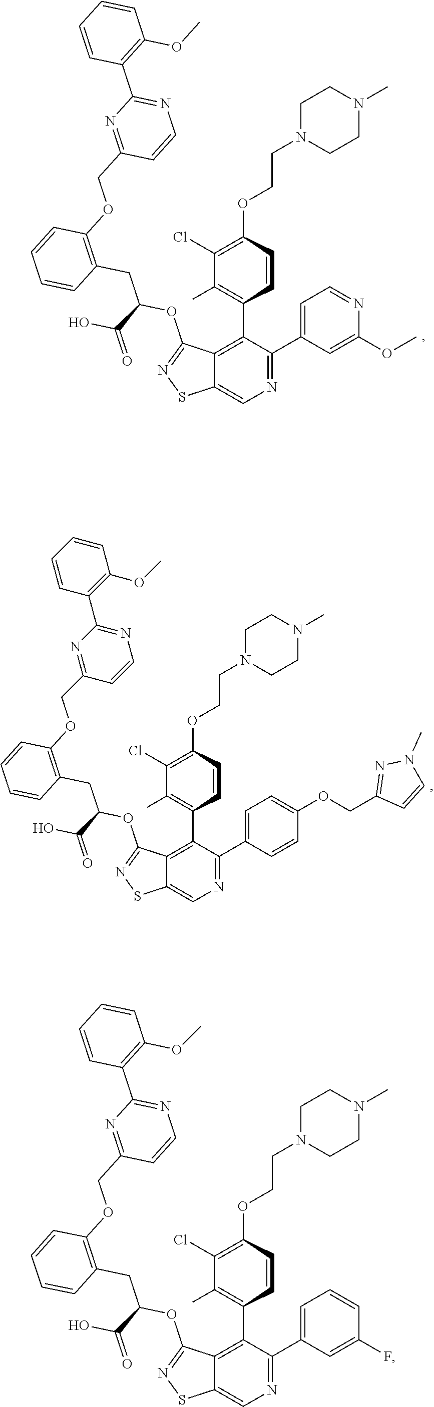

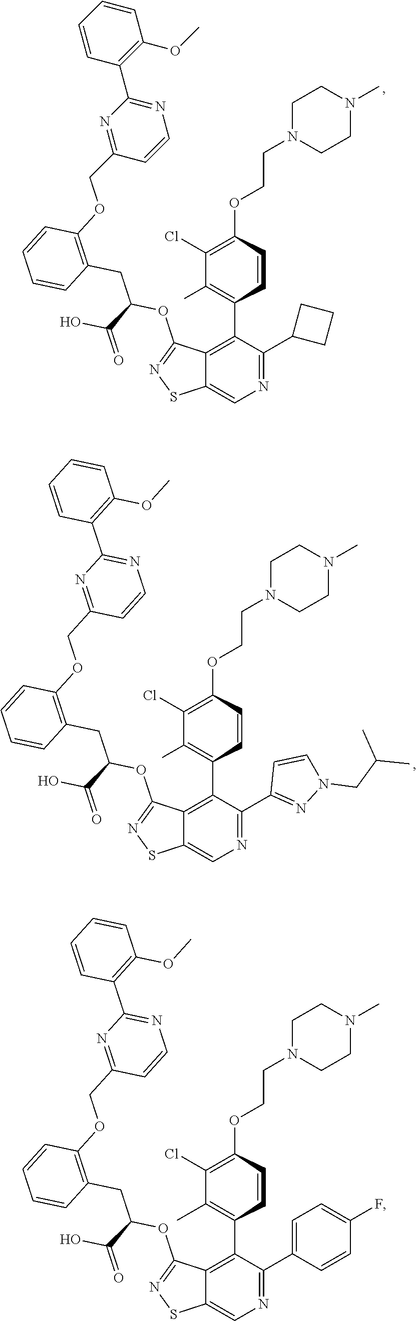

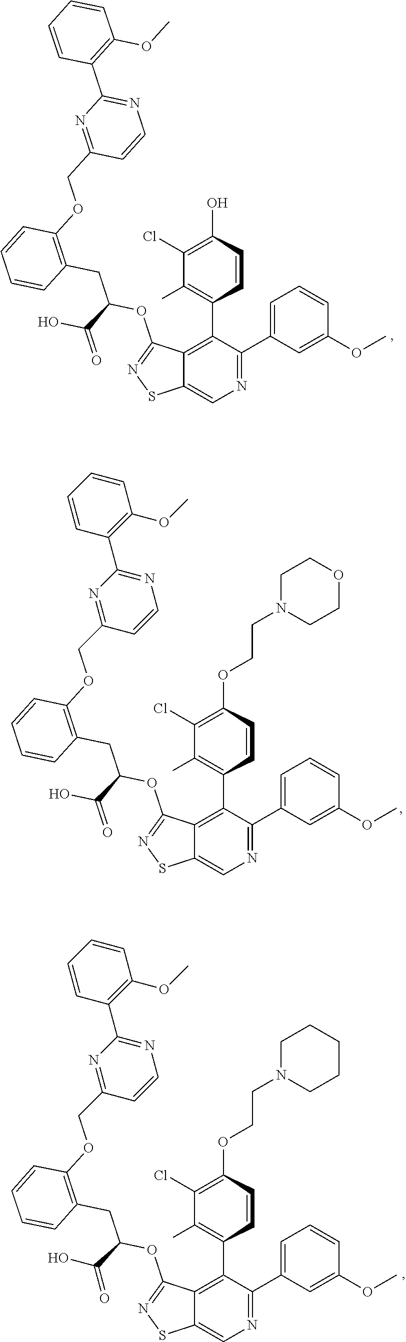

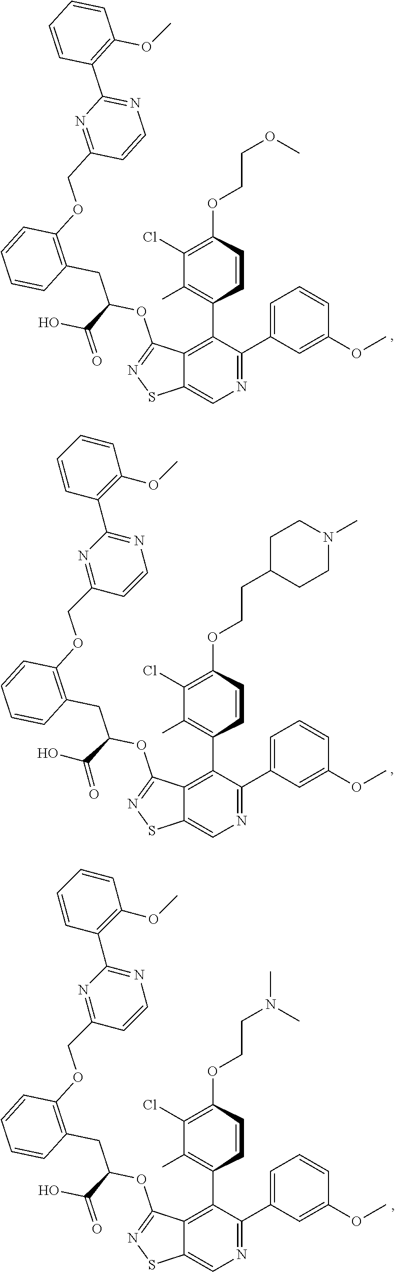

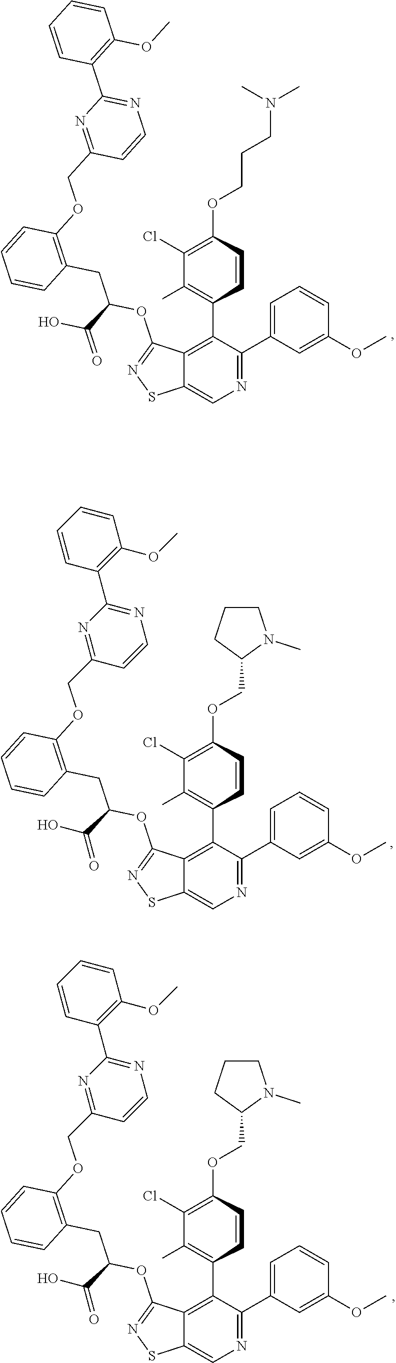

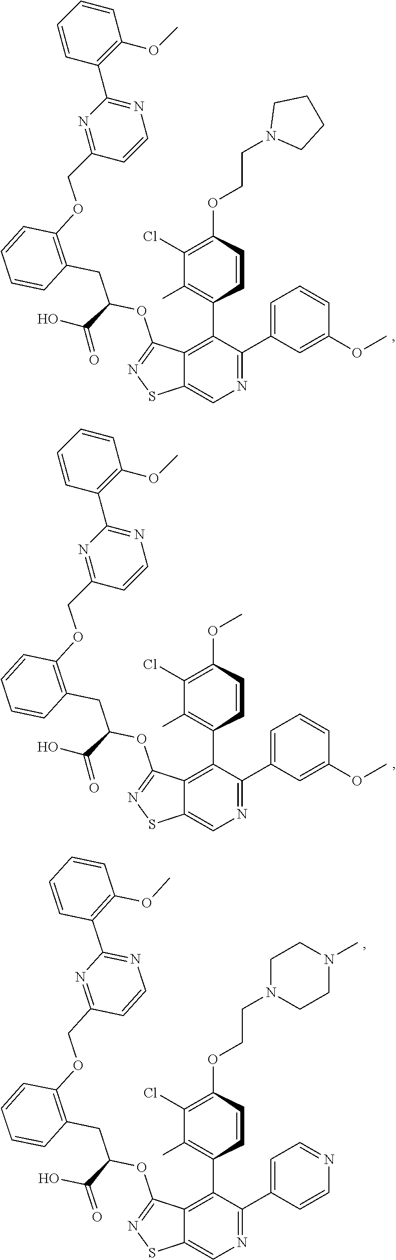

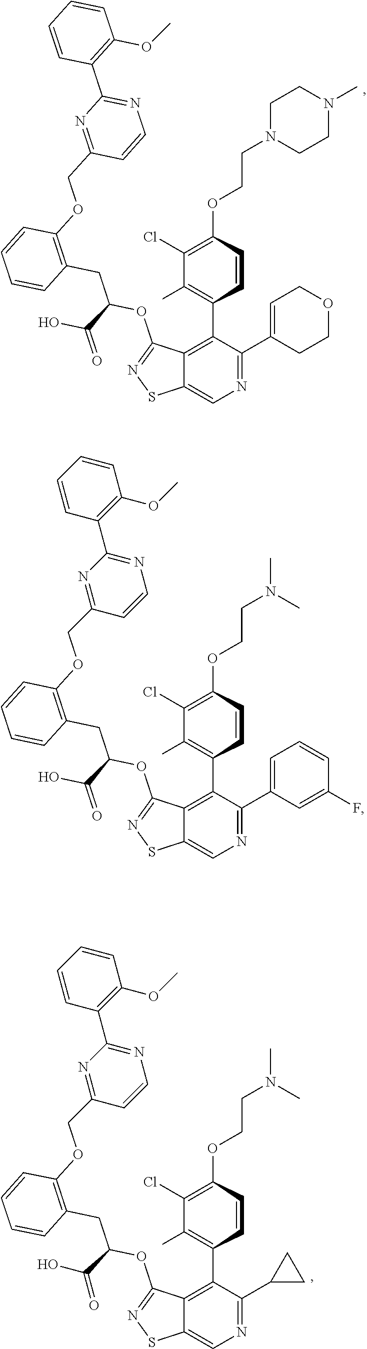

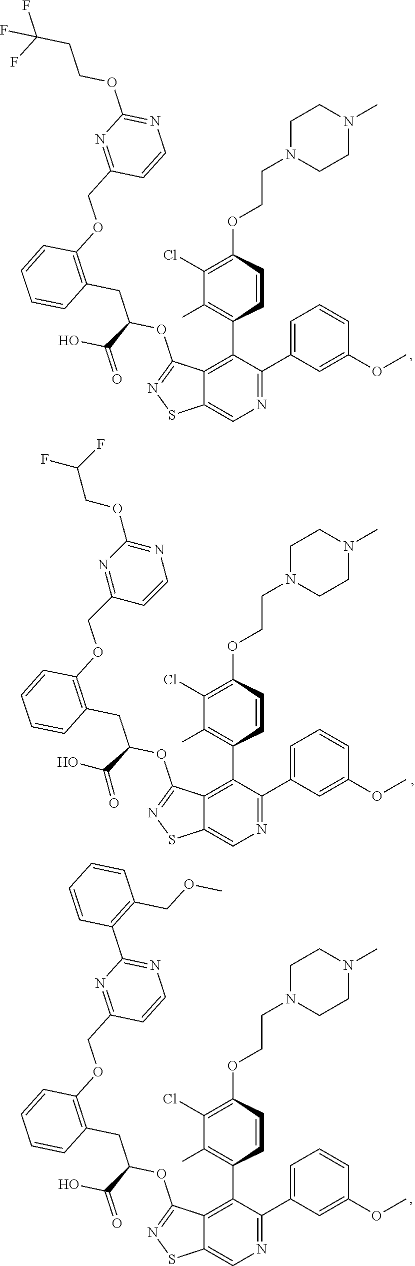

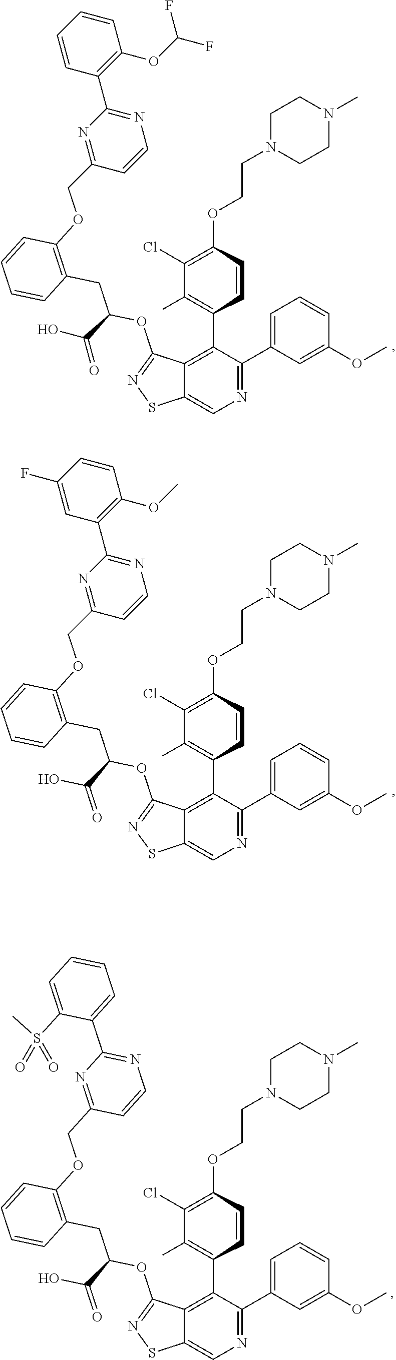

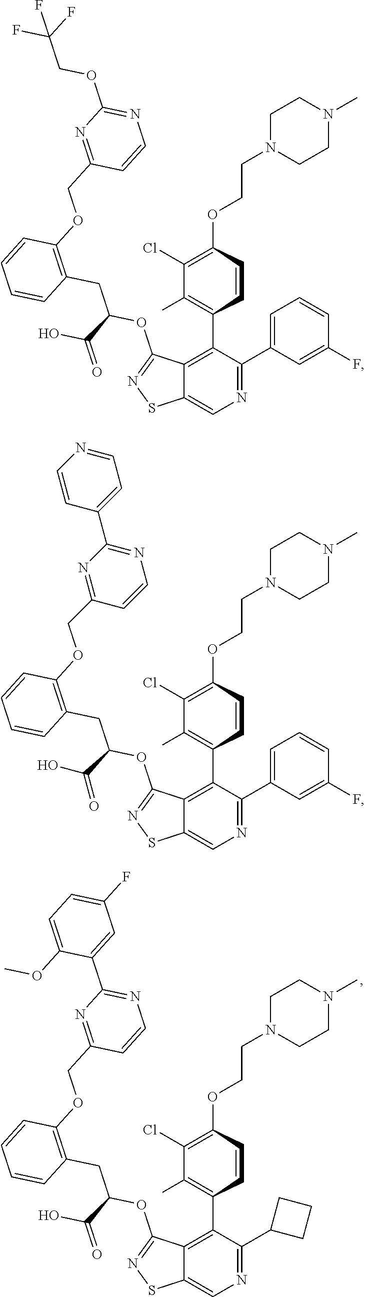

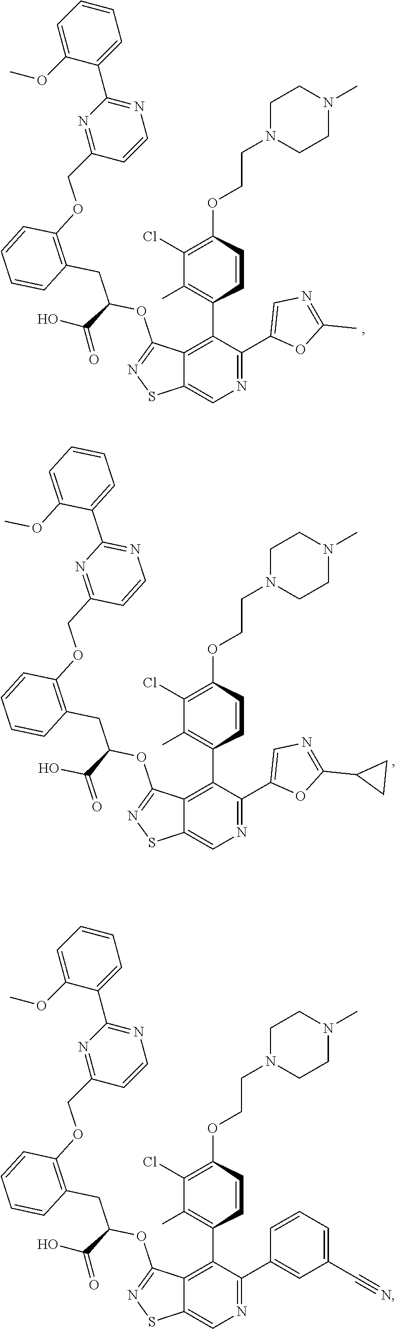

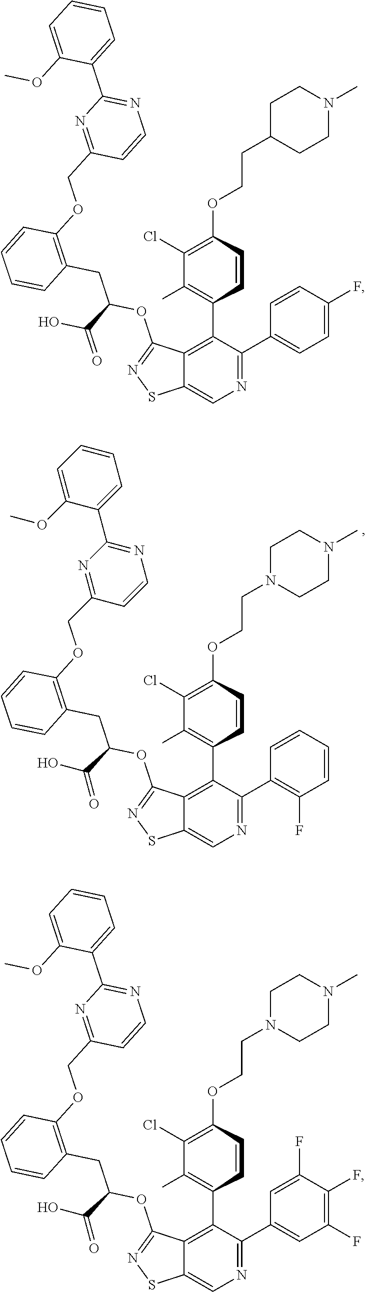

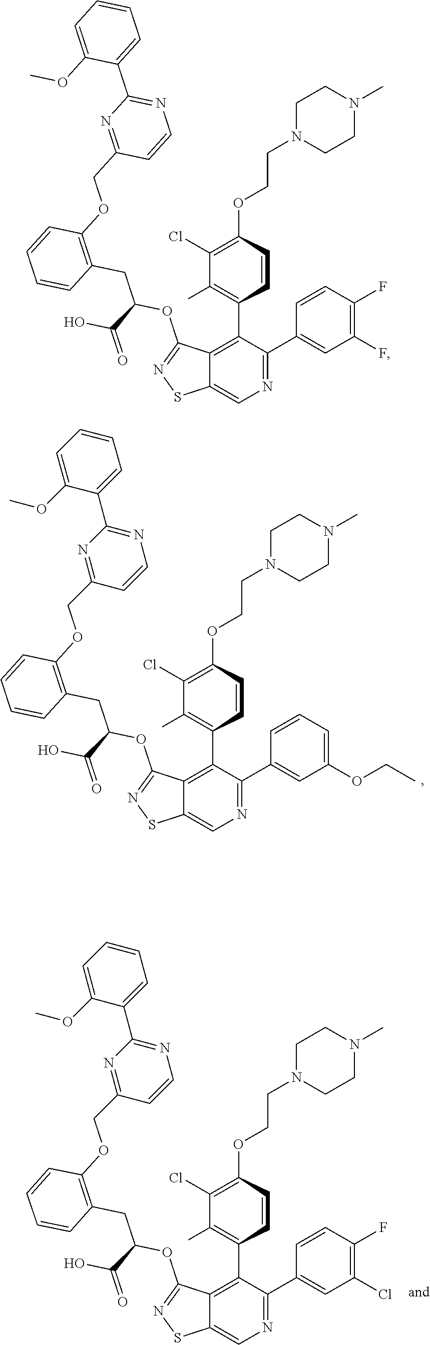

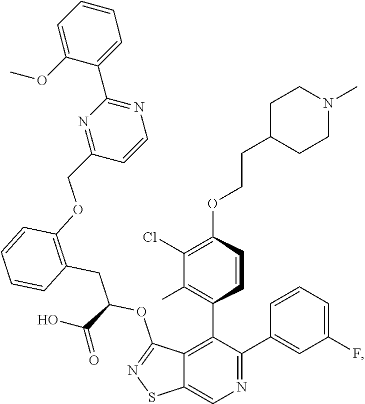

or a pharmaceutically acceptable salt thereof.

In some aspects, the invention relates to a compound of Formula II having a structure selected from:

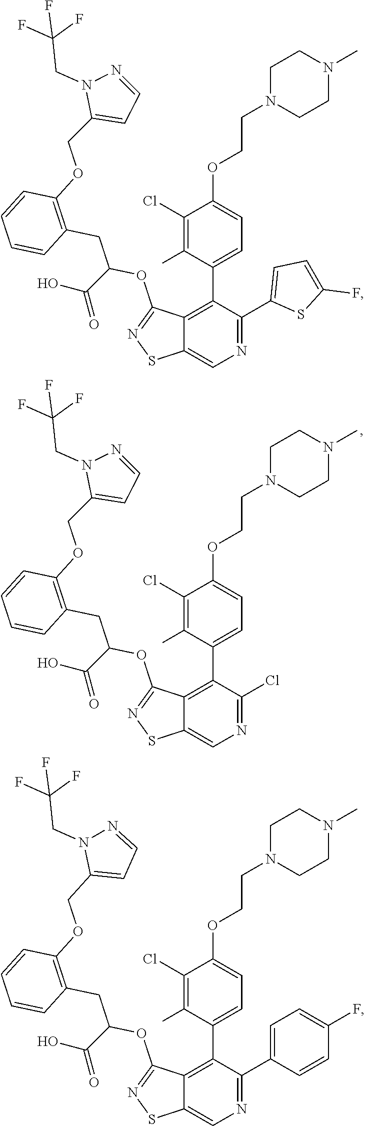 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl]

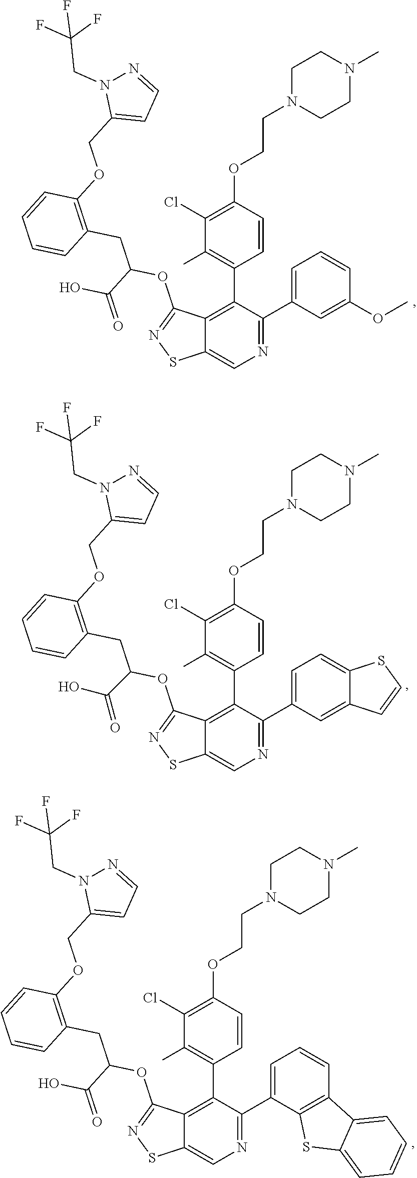 [SMILES: COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3ccc4sccc4c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3sc3ccccc34)ncc3snc(OC(Cc4ccccc4OCc4ccnn4CC(F)(F)F)C(=O)O)c23)ccc(OCCN2CCN(C)CC2)c1Cl]

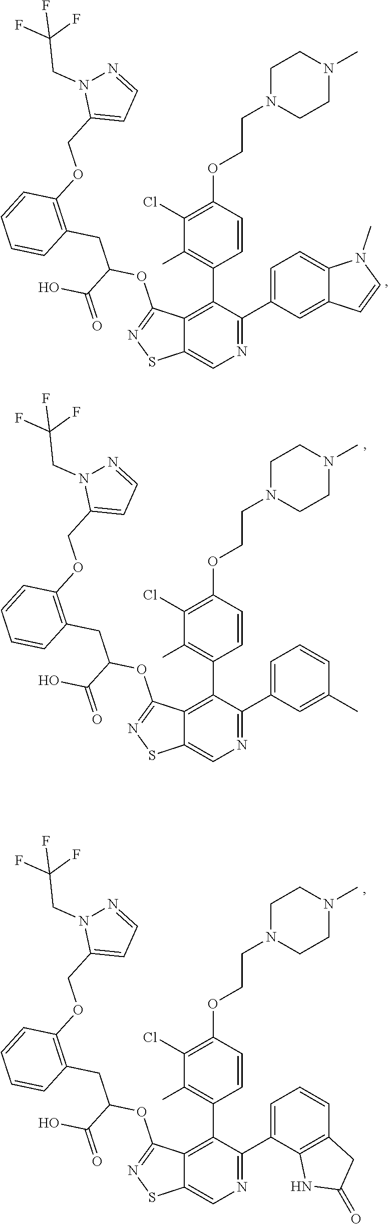 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl]

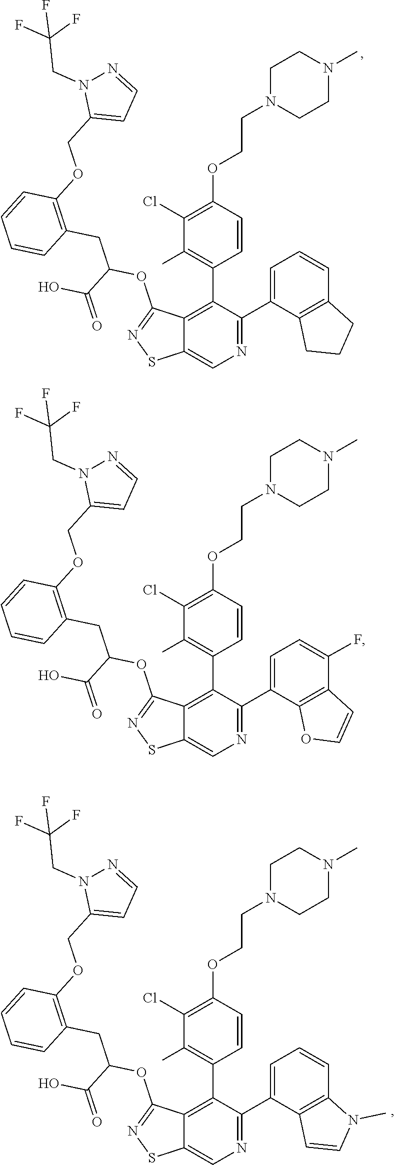 [SMILES: Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl]

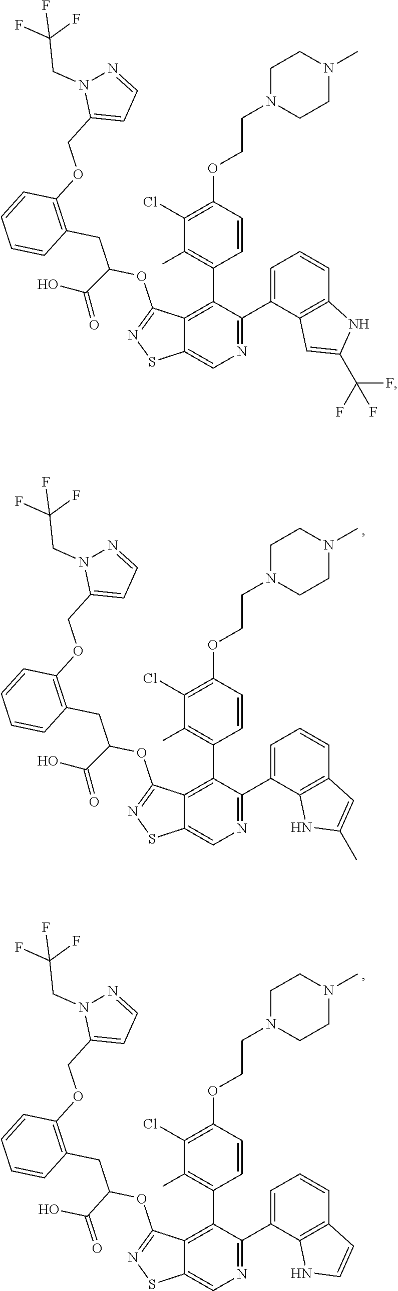 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1 ; Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl]

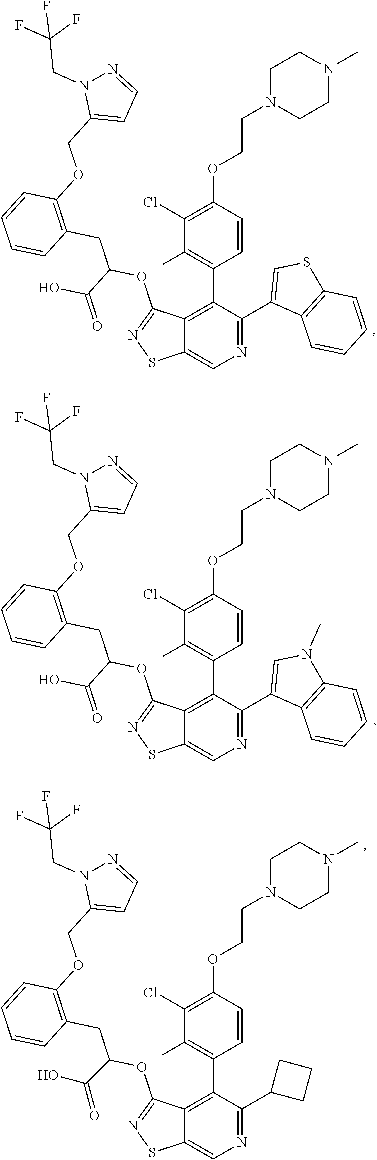 [SMILES: Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl]

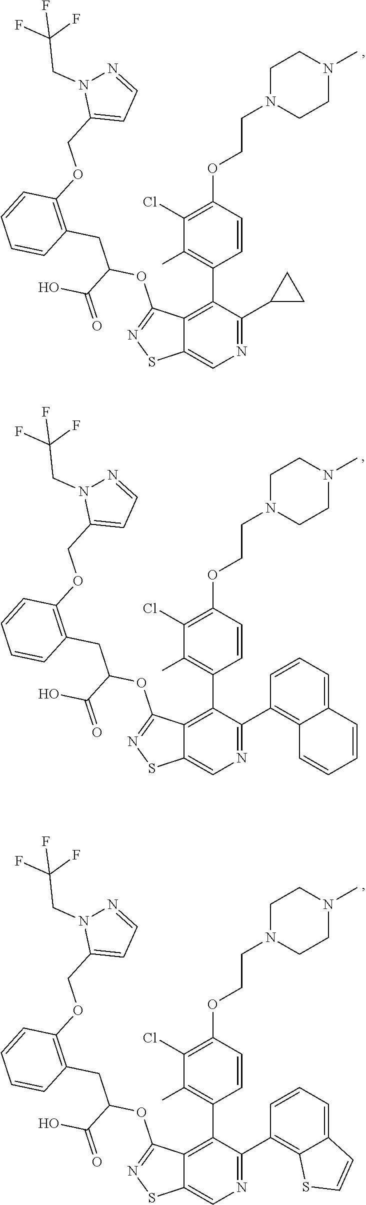 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl]

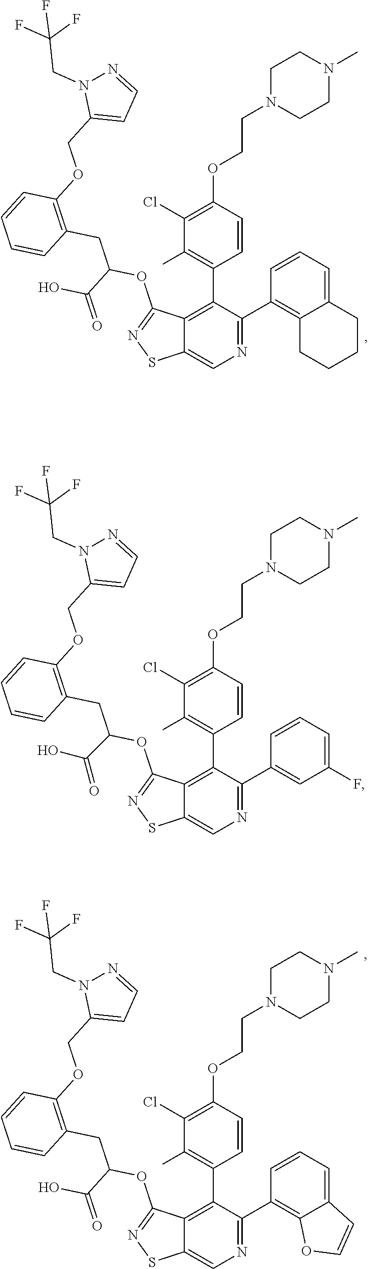 [SMILES: Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl]

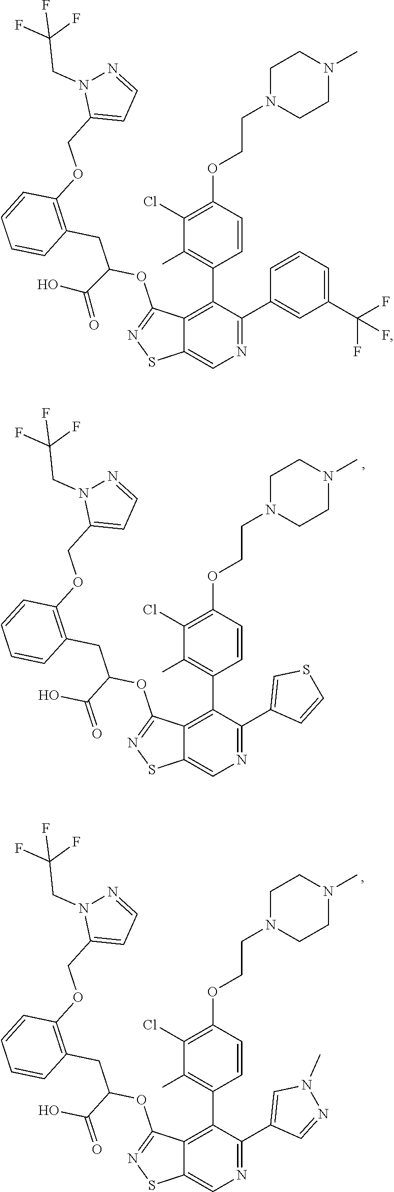 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl]

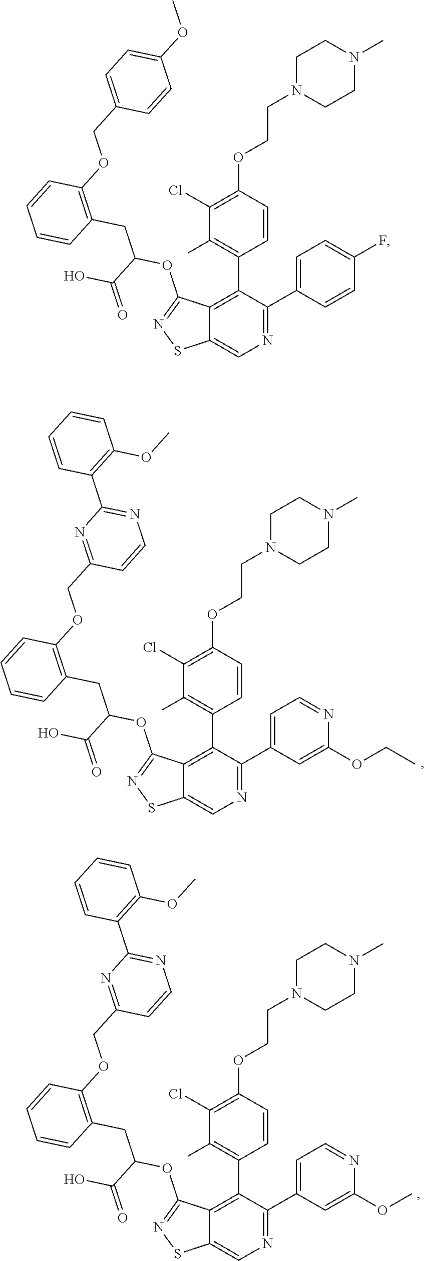 [SMILES: COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1 ; CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1]

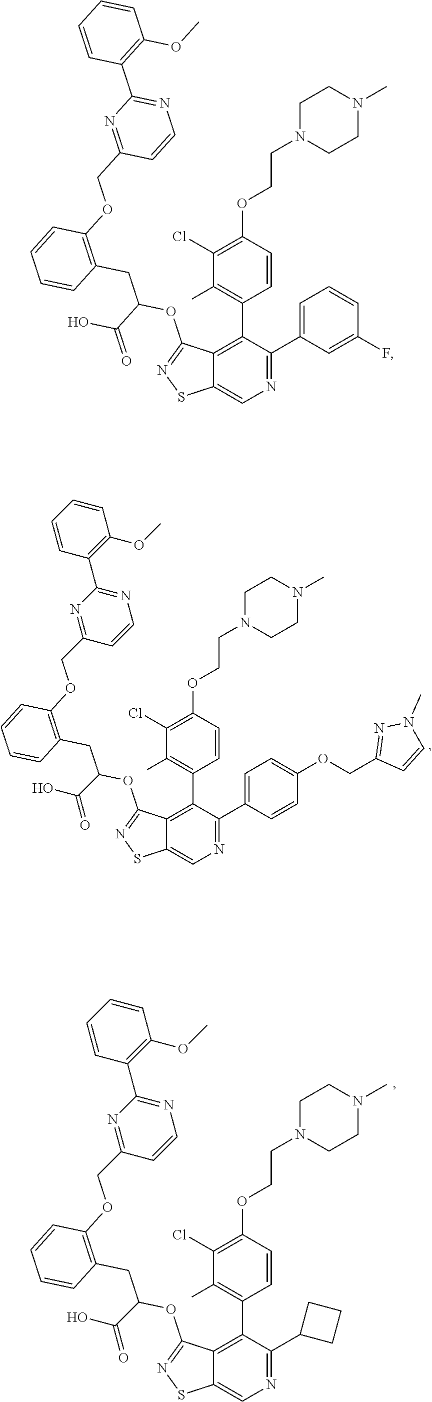 [SMILES: COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1]

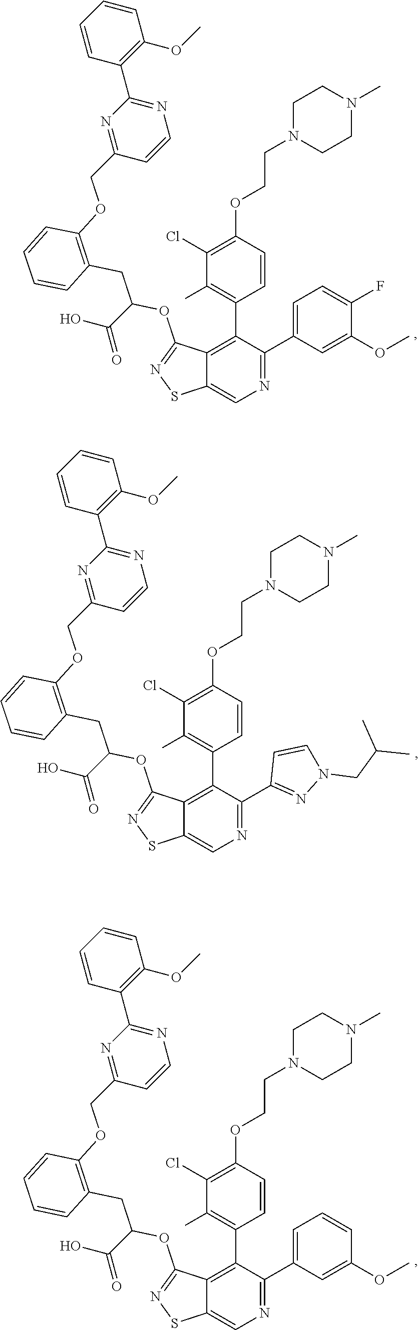 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F ; COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1]

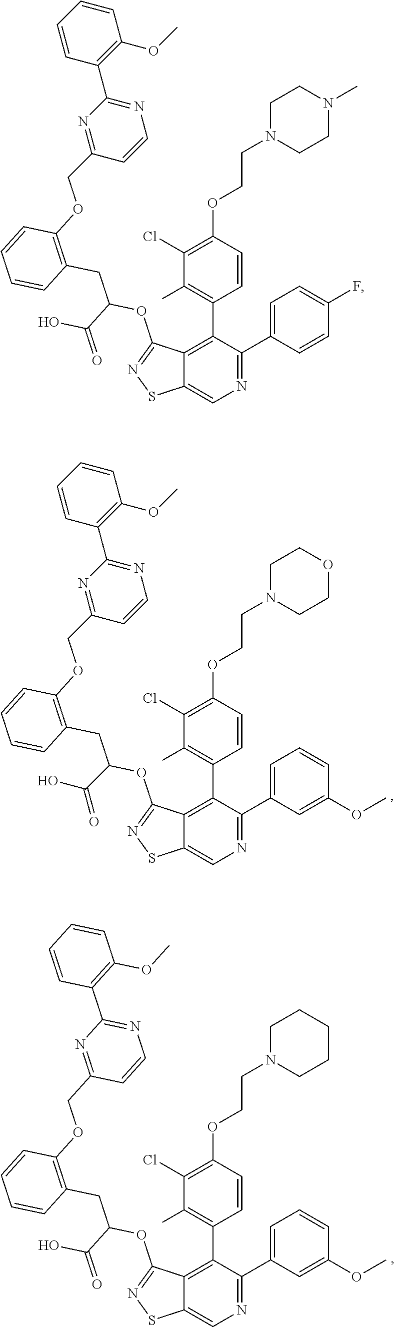 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1]

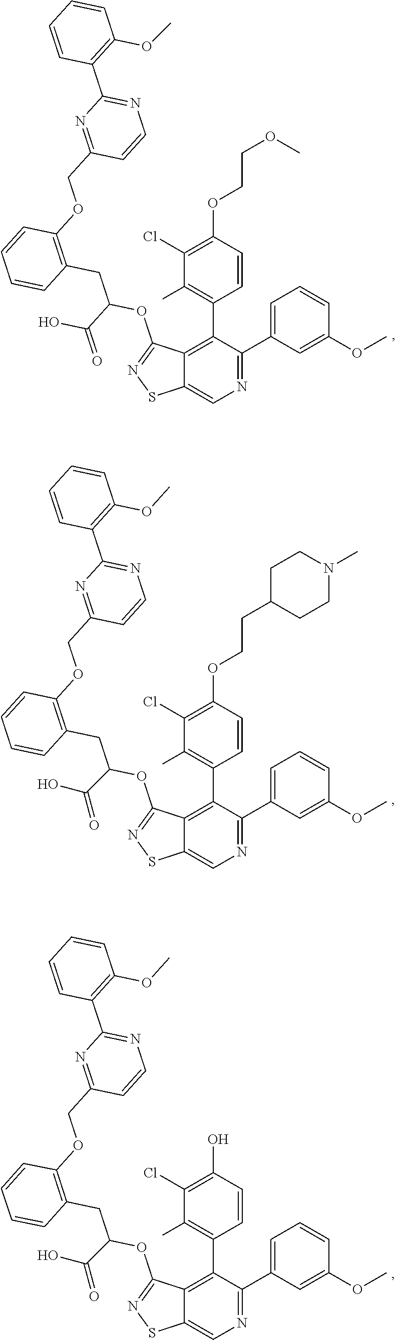 [SMILES: COCCOc1ccc(-c2c(-c3cccc(OC)c3)ncc3snc(OC(Cc4ccccc4OCc4ccnc(-c5ccccc5OC)n4)C(=O)O)c23)c(C)c1Cl ; COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1]

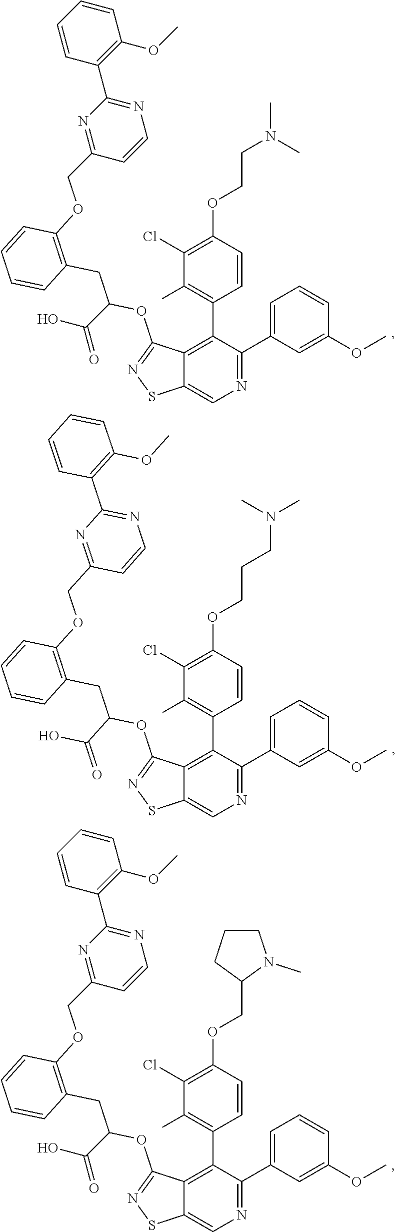

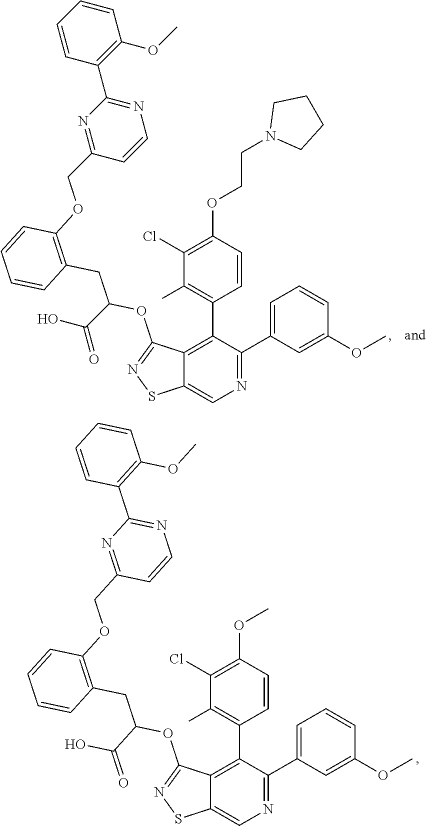

or a pharmaceutically acceptable salt thereof.

In some aspects, the invention relates to a compound of Formula II having a structure selected from:

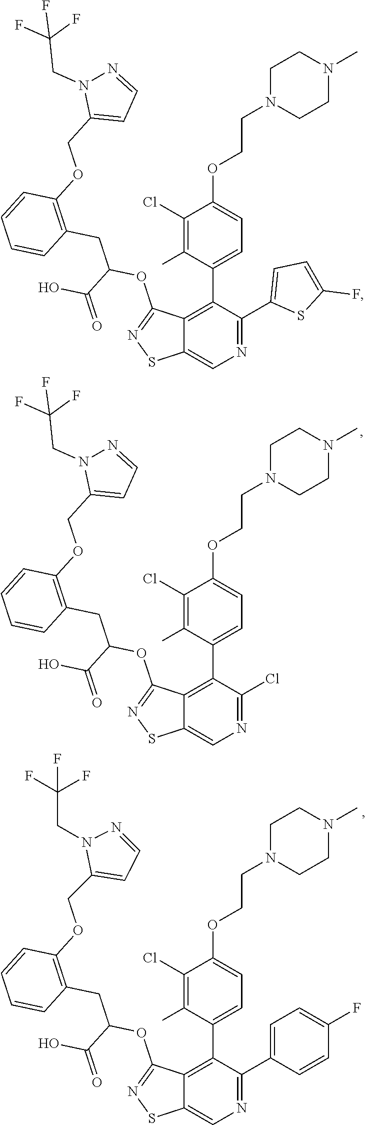 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl]

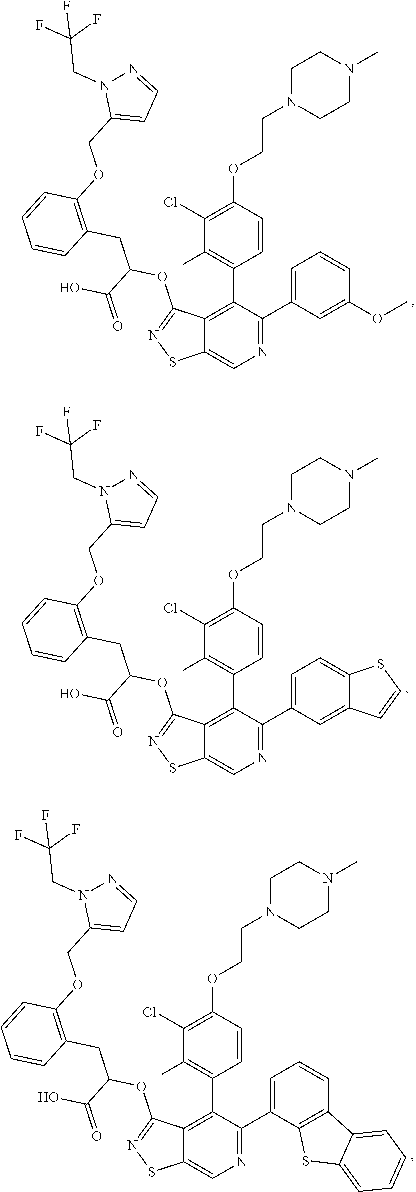 [SMILES: COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3ccc4sccc4c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3sc3ccccc34)ncc3snc(OC(Cc4ccccc4OCc4ccnn4CC(F)(F)F)C(=O)O)c23)ccc(OCCN2CCN(C)CC2)c1Cl]

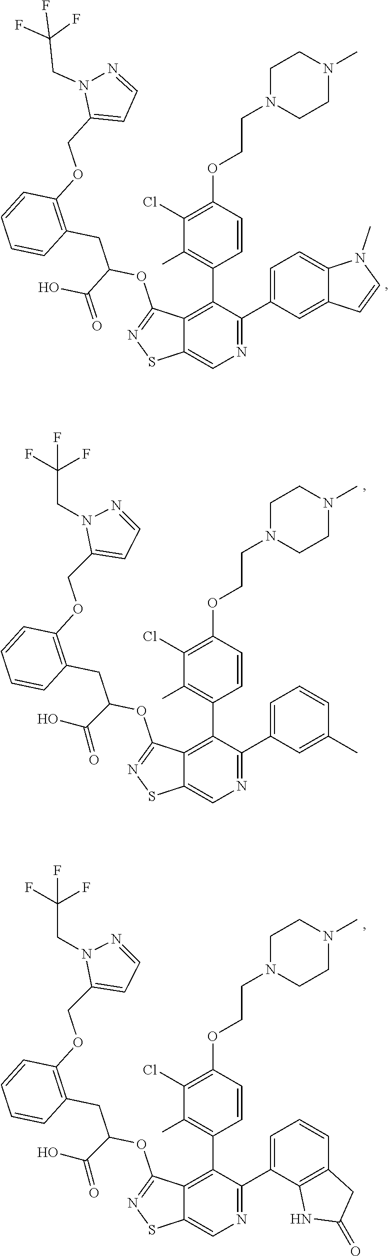 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl]

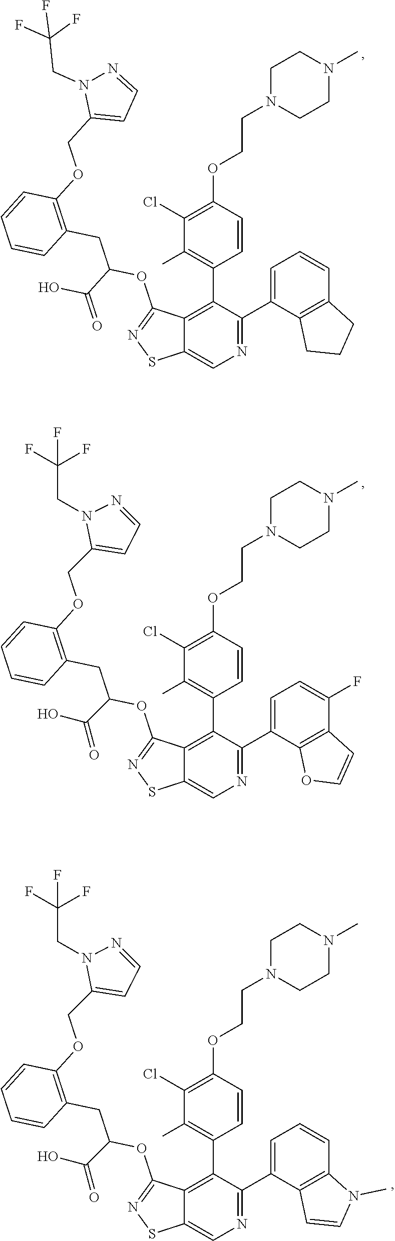 [SMILES: Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl]

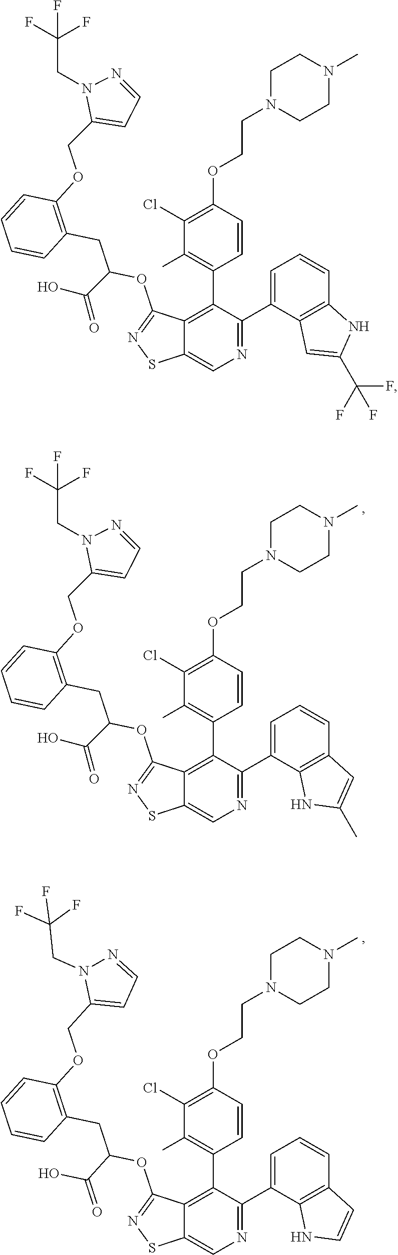 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1 ; Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl]

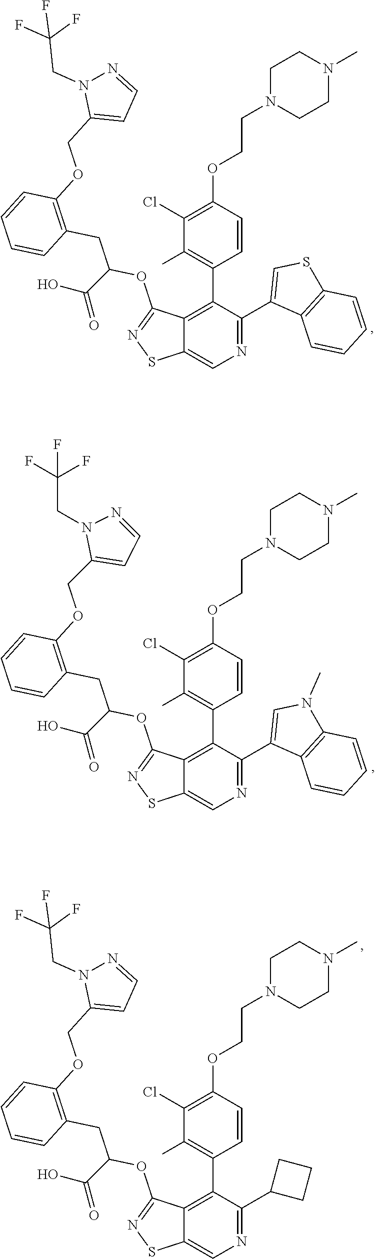 [SMILES: Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl]

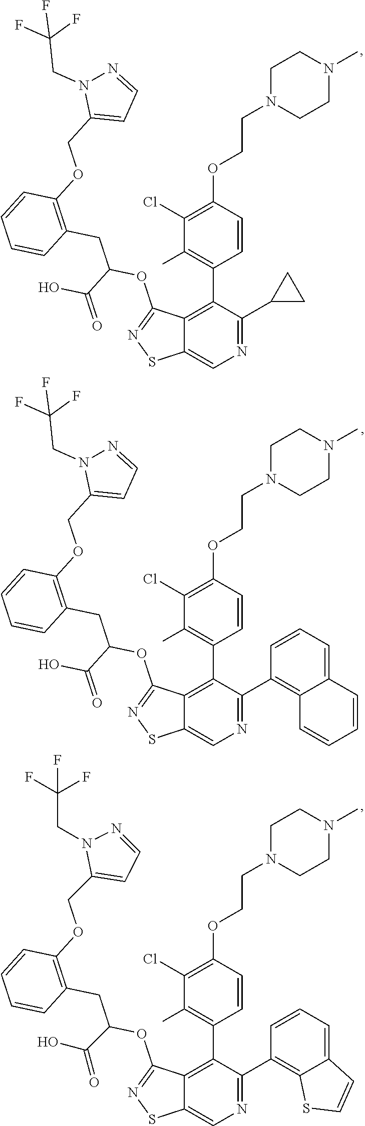 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl]

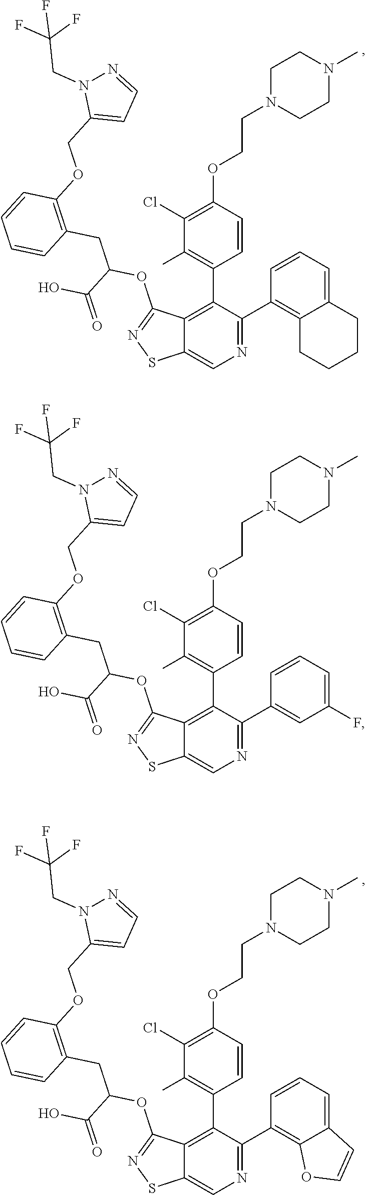 [SMILES: Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl]

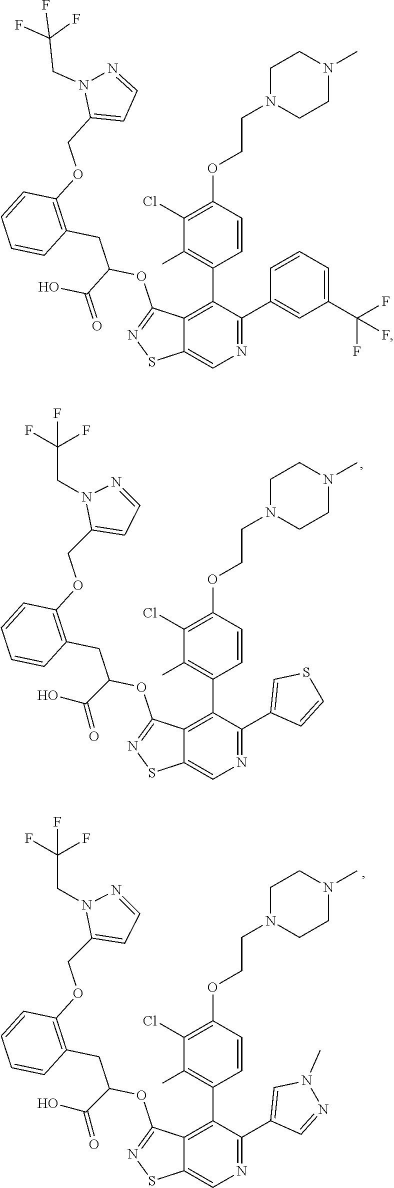 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl]

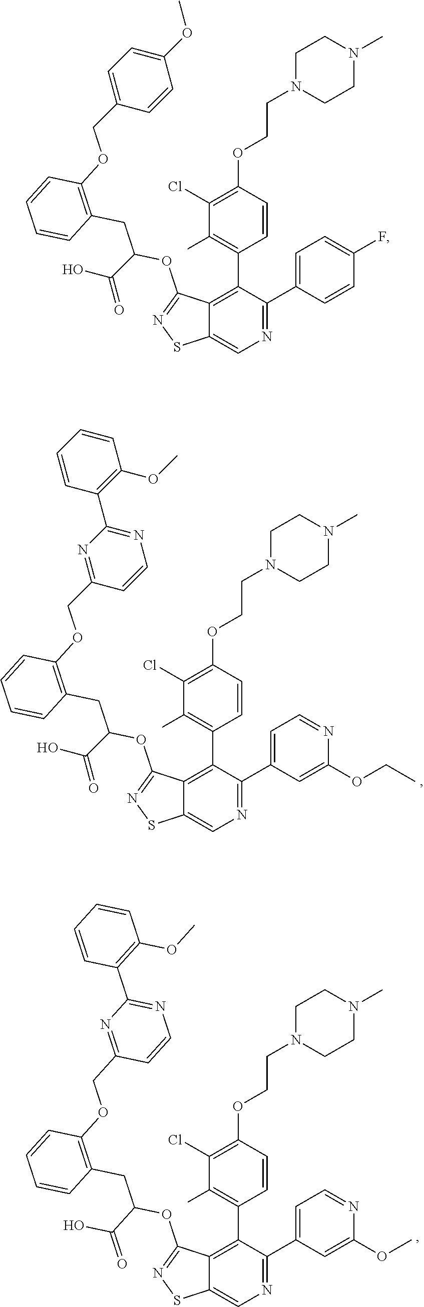 [SMILES: COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1 ; CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1]

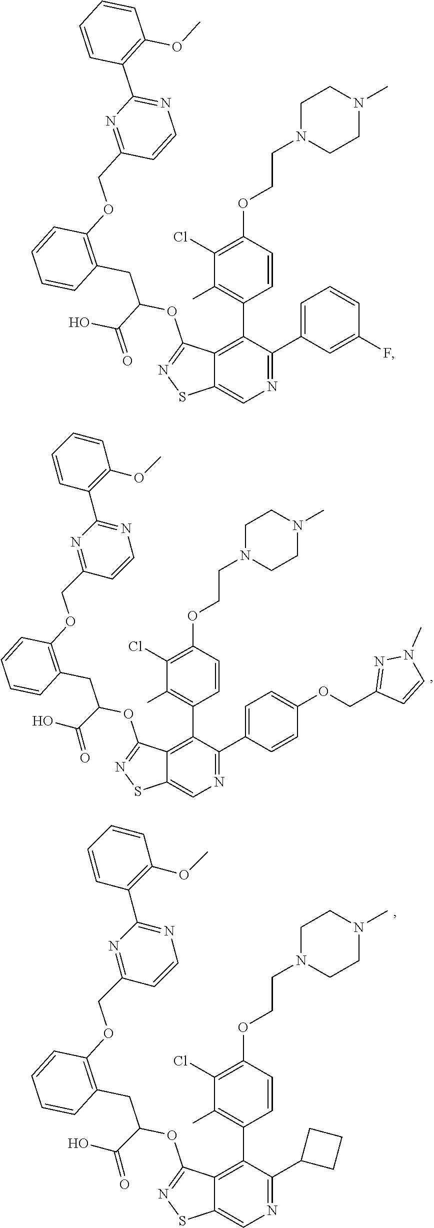

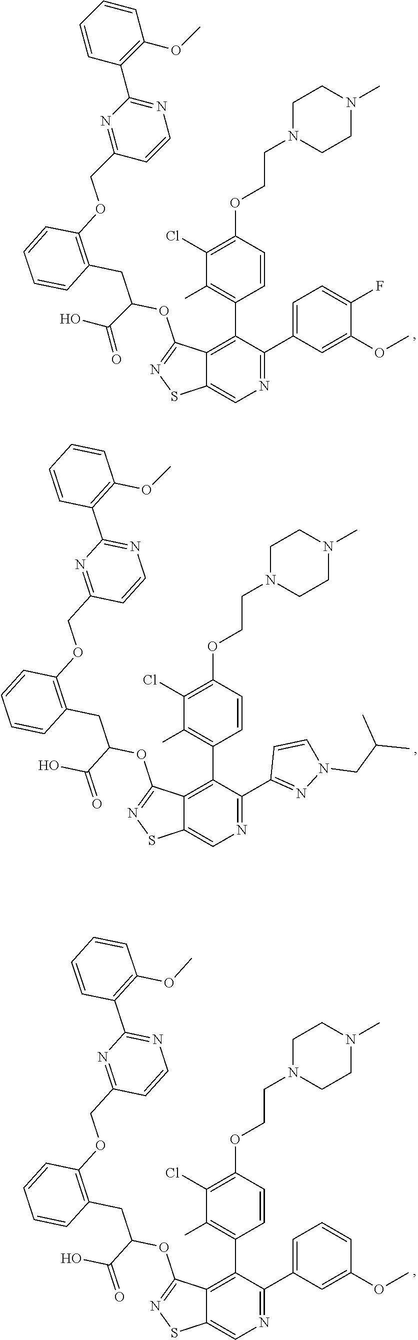 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F ; COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1]

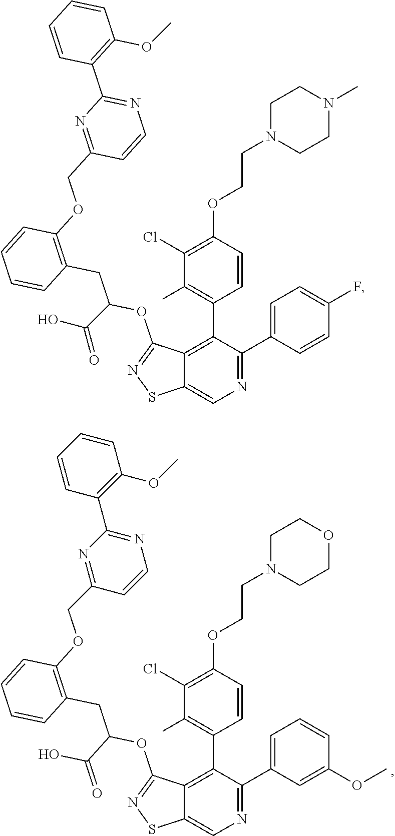 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1]

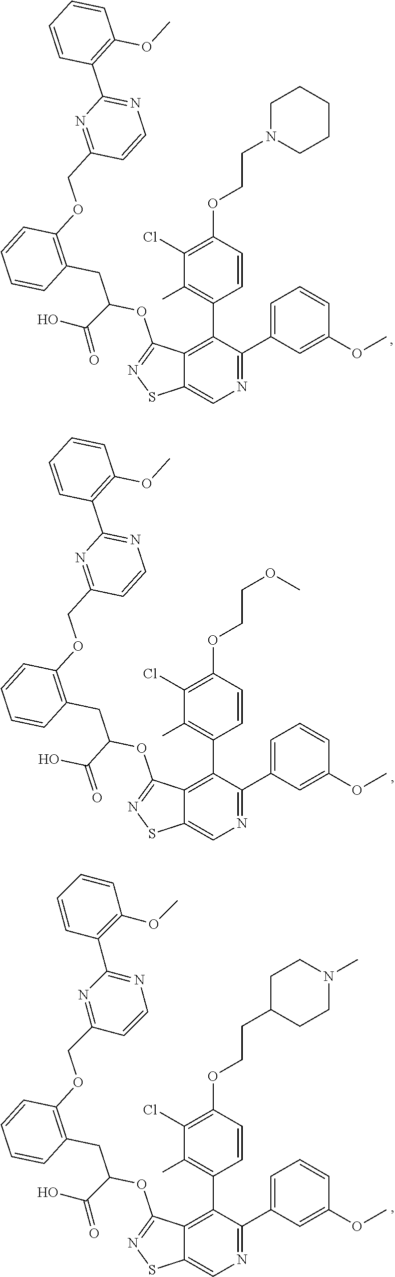 [SMILES: COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl ; COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1]

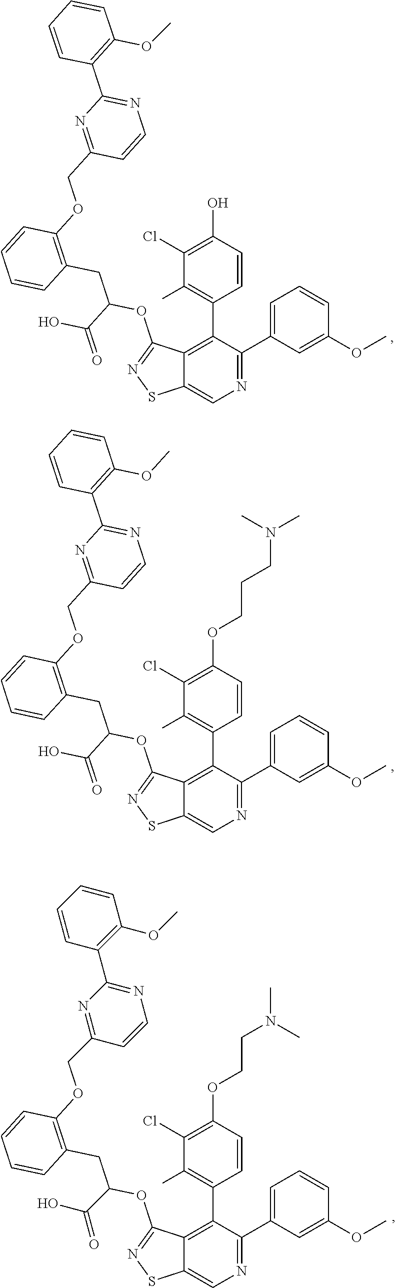 [SMILES: COc1cccc(-c2ncc3snc(OC(Cc4ccccc4OCc4ccnc(-c5ccccc5OC)n4)C(=O)O)c3c2-c2ccc(O)c(Cl)c2C)c1 ; COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1]

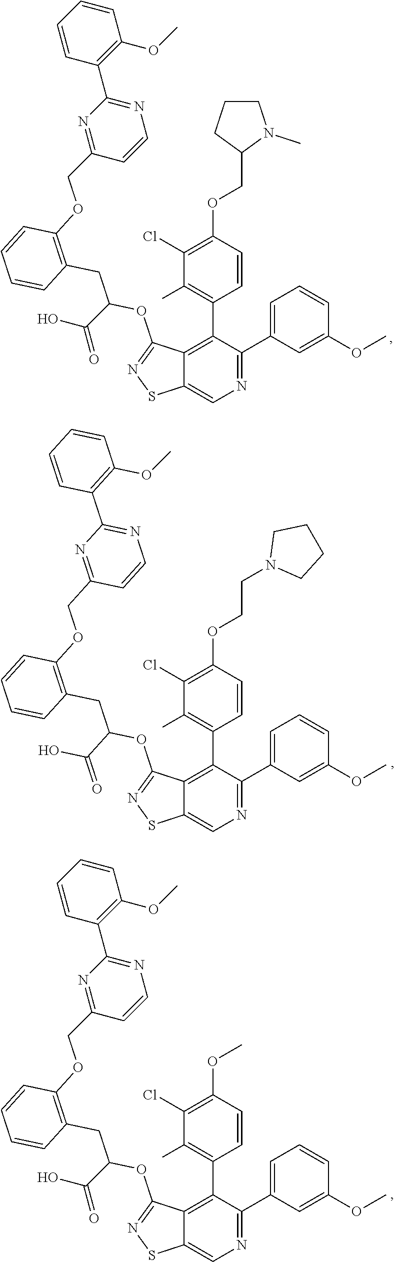 [SMILES: COc1cccc(-c2ncc3snc(OC(Cc4ccccc4OCc4ccnc(-c5ccccc5OC)n4)C(=O)O)c3c2-c2ccc(OCC3CCCN3C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OC)c(Cl)c2C)c1]

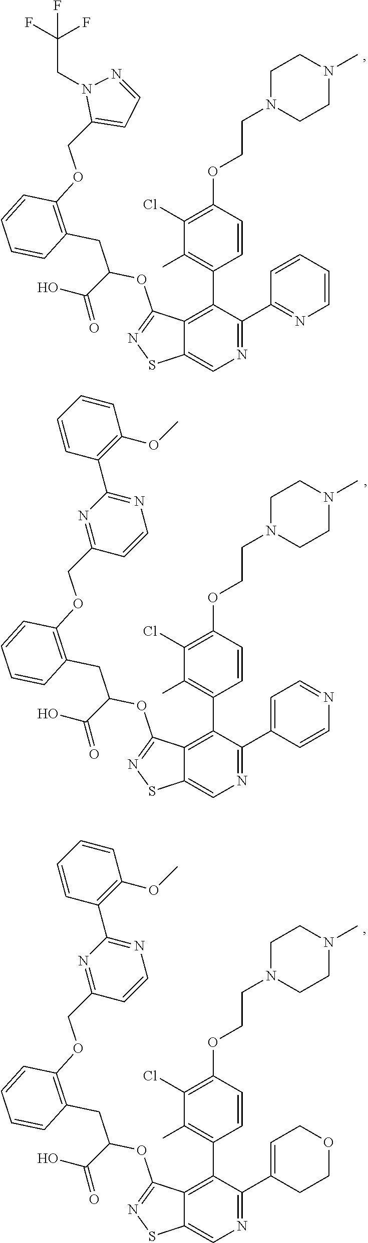 [SMILES: Cc1c(-c2c(-c3ccccn3)ncc3snc(OC(Cc4ccccc4OCc4ccnn4CC(F)(F)F)C(=O)O)c23)ccc(OCCN2CCN(C)CC2)c1Cl ; COc1ccccc1-c1nccc(COc2ccccc2CC(Oc2nsc3cnc(-c4ccncc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4=CCOCC4)c(-c4ccc(OCCN5CCN(C)CC5)c(Cl)c4C)c23)C(=O)O)n1]

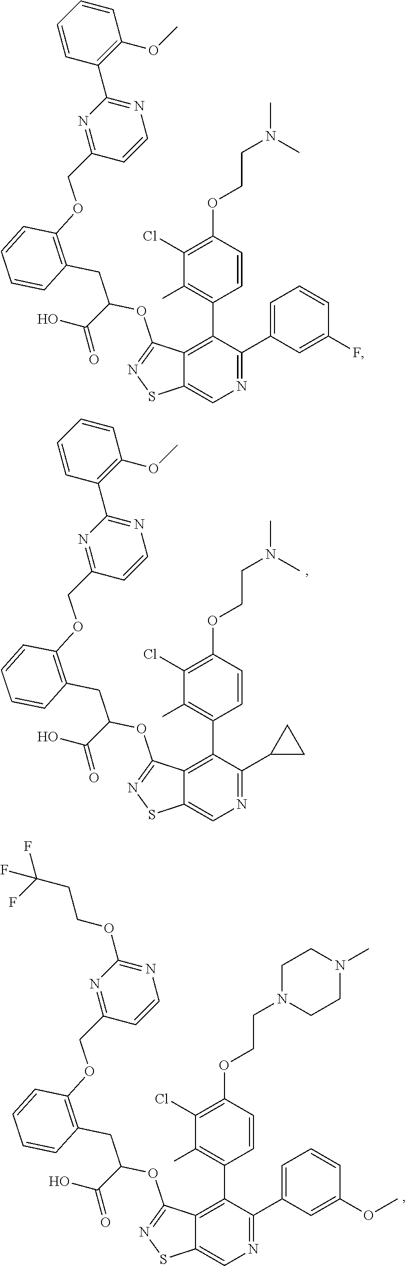 [SMILES: COc1ccccc1-c1nccc(COc2ccccc2CC(Oc2nsc3cnc(-c4cccc(F)c4)c(-c4ccc(OCCN(C)C)c(Cl)c4C)c23)C(=O)O)n1 ; COc1ccccc1-c1nccc(COc2ccccc2CC(Oc2nsc3cnc(C4CC4)c(-c4ccc(OCCN(C)C)c(Cl)c4C)c23)C(=O)O)n1 ; COc1cccc(-c2ncc3snc(OC(Cc4ccccc4OCc4ccnc(OCCC(F)(F)F)n4)C(=O)O)c3c2-c2ccc(OCCN3CCN(C)CC3)c(Cl)c2C)c1]

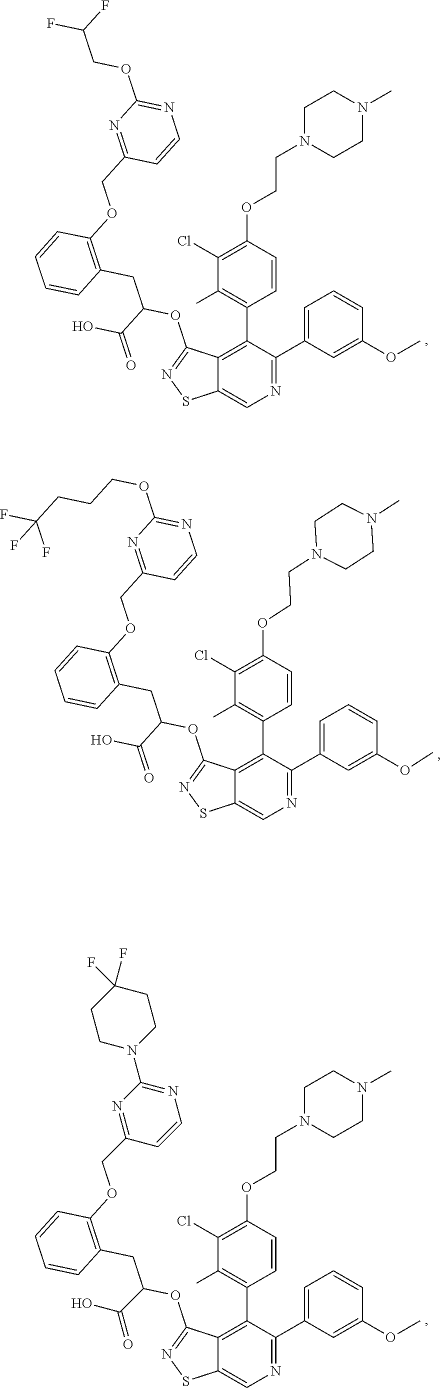 [SMILES: COc1cccc(-c2ncc3snc(OC(Cc4ccccc4OCc4ccnc(OCC(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(OCCCC(F)(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(F)(F)CC5)n4)C(=O)O)c3c2-c2ccc(OCCN3CCN(C)CC3)c(Cl)c2C)c1]

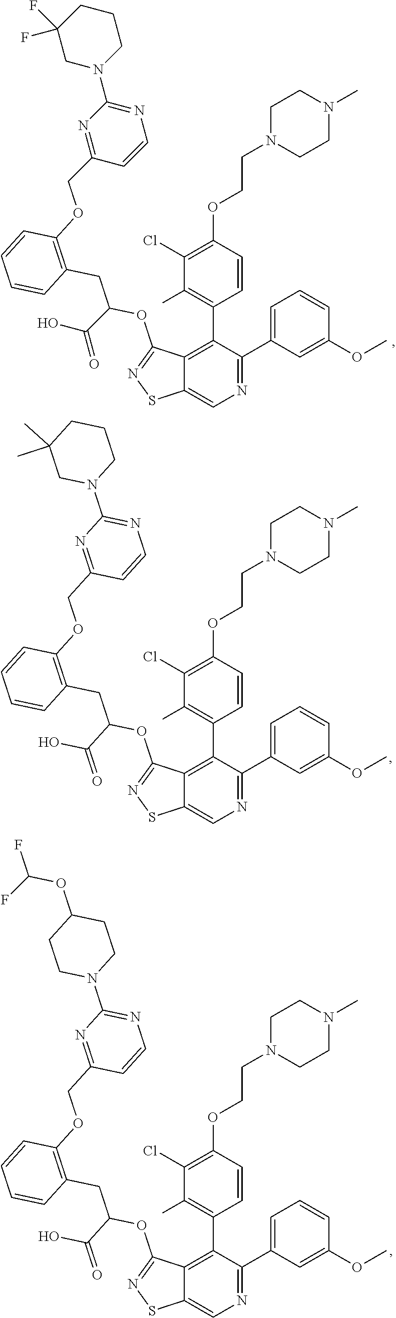 [SMILES: COc1cccc(-c2ncc3snc(OC(Cc4ccccc4OCc4ccnc(N5CCCC(F)(F)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCCC(C)(C)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(OC(F)F)CC5)n4)C(=O)O)c3c2-c2ccc(OCCN3CCN(C)CC3)c(Cl)c2C)c1]

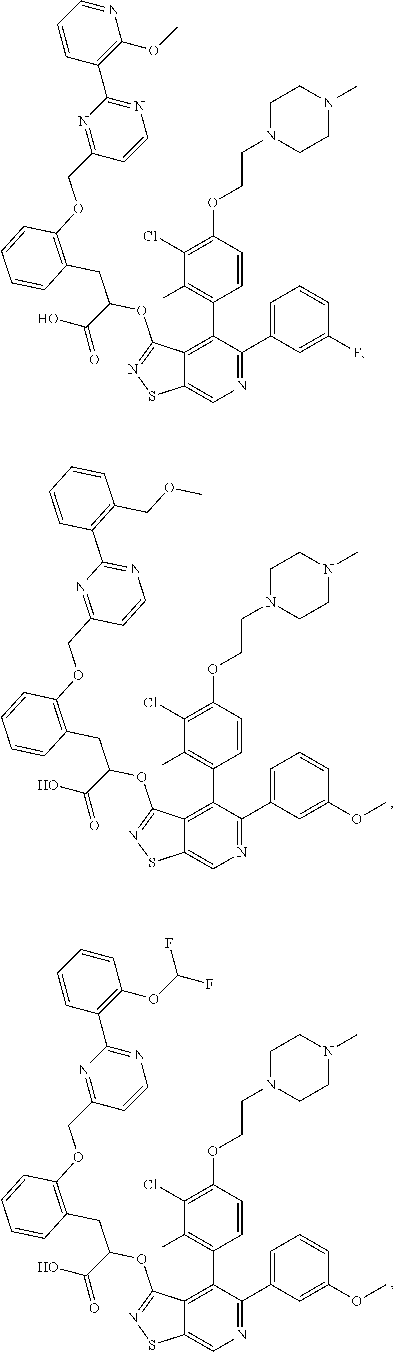 [SMILES: COc1ncccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1 ; COCc1ccccc1-c1nccc(COc2ccccc2CC(Oc2nsc3cnc(-c4cccc(OC)c4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC(F)F)n4)C(=O)O)c3c2-c2ccc(OCCN3CCN(C)CC3)c(Cl)c2C)c1]

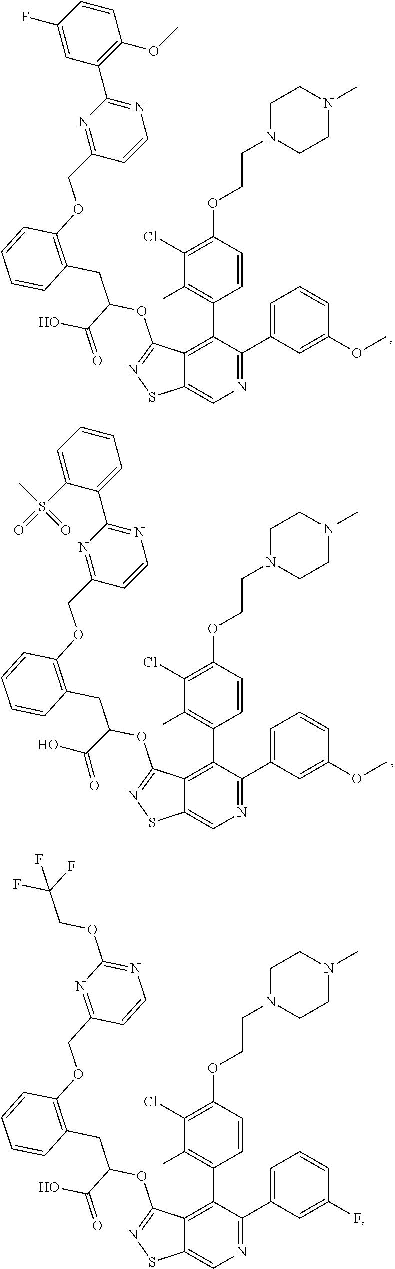 [SMILES: COc1cccc(-c2ncc3snc(OC(Cc4ccccc4OCc4ccnc(-c5cc(F)ccc5OC)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(-c5ccccc5S(C)(=O)=O)n4)C(=O)O)c3c2-c2ccc(OCCN3CCN(C)CC3)c(Cl)c2C)c1 ; Cc1c(-c2c(-c3cccc(F)c3)ncc3snc(OC(Cc4ccccc4OCc4ccnc(OCC(F)(F)F)n4)C(=O)O)c23)ccc(OCCN2CCN(C)CC2)c1Cl]

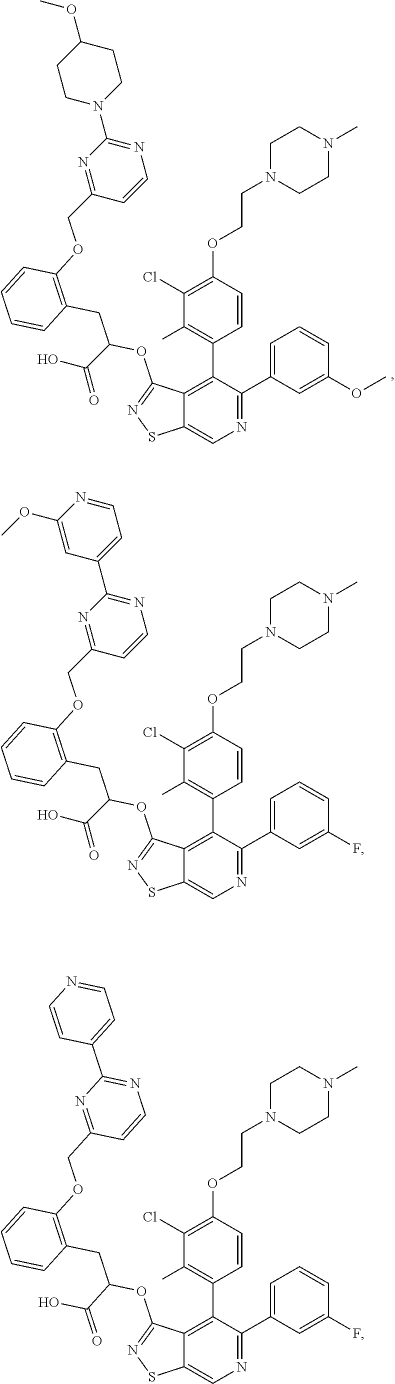 [SMILES: COc1cccc(-c2ncc3snc(OC(Cc4ccccc4OCc4ccnc(N5CCC(OC)CC5)n4)C(=O)O)c3c2-c2ccc(OCCN3CCN(C)CC3)c(Cl)c2C)c1 ; COc1cc(-c2nccc(COc3ccccc3CC(Oc3nsc4cnc(-c5cccc(F)c5)c(-c5ccc(OCCN6CCN(C)CC6)c(Cl)c5C)c34)C(=O)O)n2)ccn1 ; Cc1c(-c2c(-c3cccc(F)c3)ncc3snc(OC(Cc4ccccc4OCc4ccnc(-c5ccncc5)n4)C(=O)O)c23)ccc(OCCN2CCN(C)CC2)c1Cl]

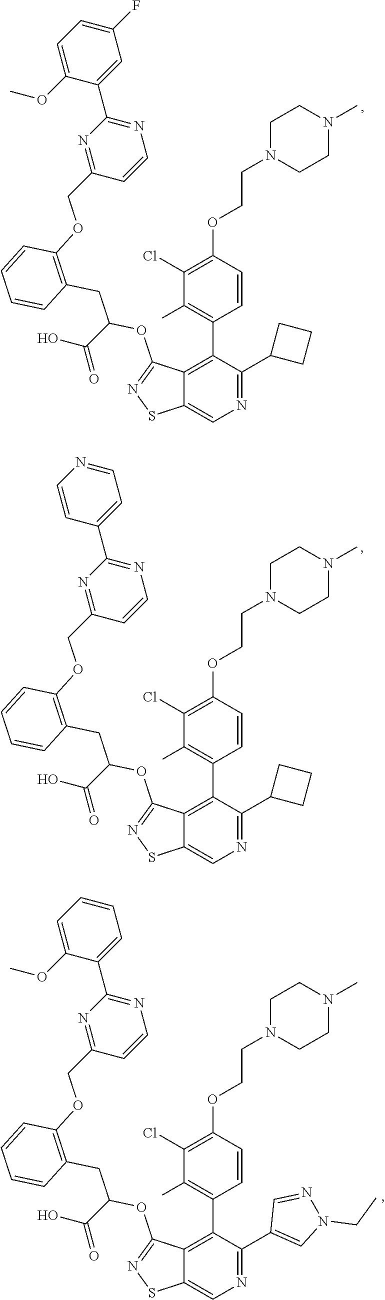 [SMILES: COc1ccc(F)cc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5ccncc5)n4)C(=O)O)c23)ccc(OCCN2CCN(C)CC2)c1Cl ; CCn1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)cn1]

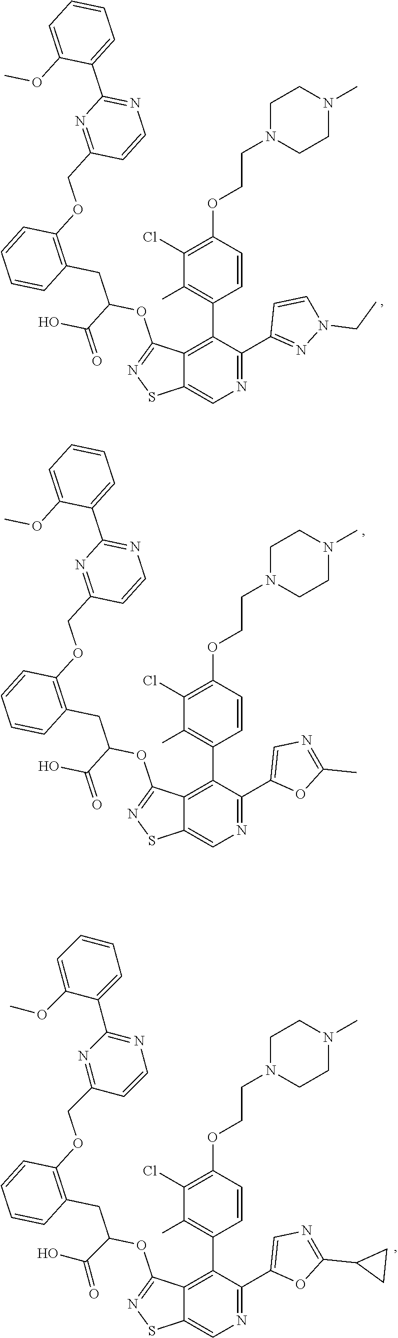 [SMILES: CCn1ccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)n1 ; COc1ccccc1-c1nccc(COc2ccccc2CC(Oc2nsc3cnc(-c4cnc(C)o4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnc(C5CC5)o4)c(-c4ccc(OCCN5CCN(C)CC5)c(Cl)c4C)c23)C(=O)O)n1]

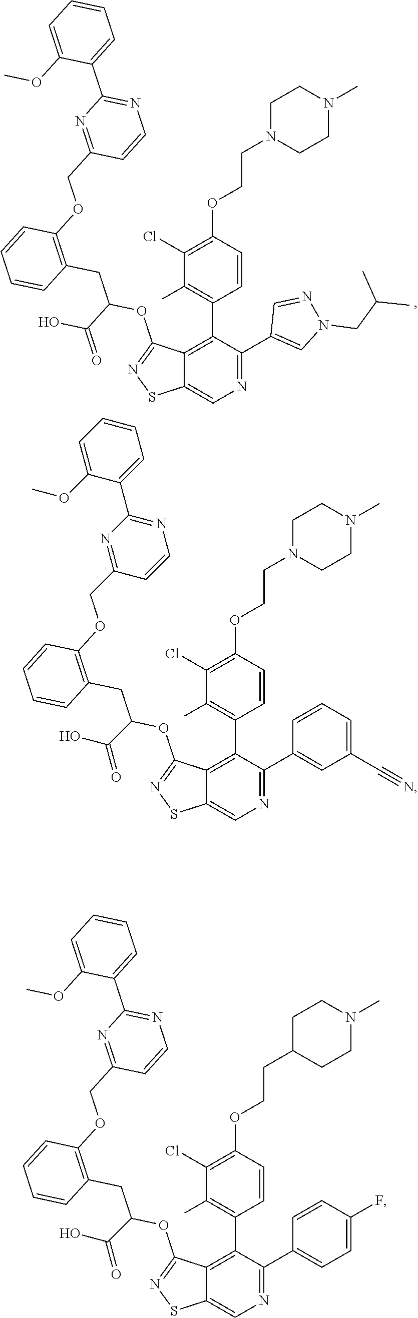

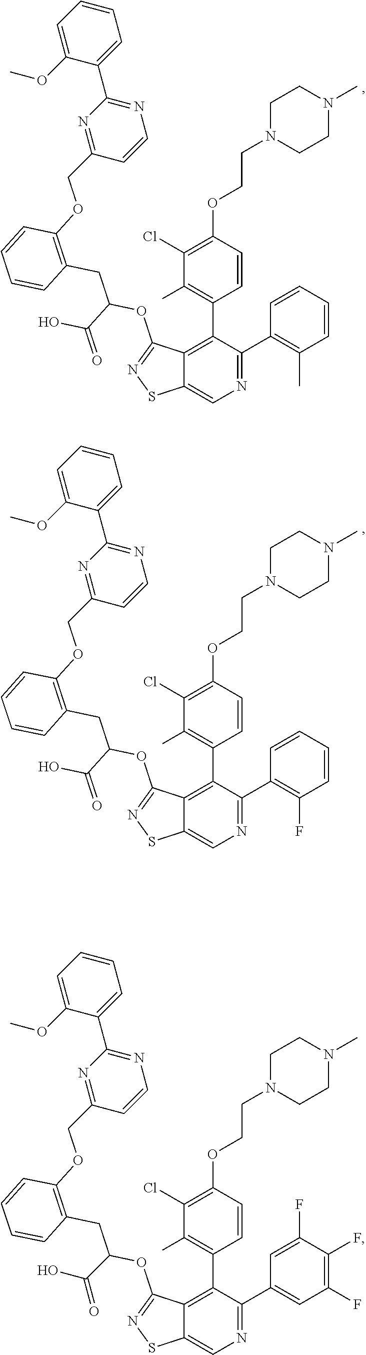 [SMILES: COc1ccccc1-c1nccc(COc2ccccc2CC(Oc2nsc3cnc(-c4ccccc4C)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccccc4F)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cc(F)c(F)c(F)c4)c(-c4ccc(OCCN5CCN(C)CC5)c(Cl)c4C)c23)C(=O)O)n1]

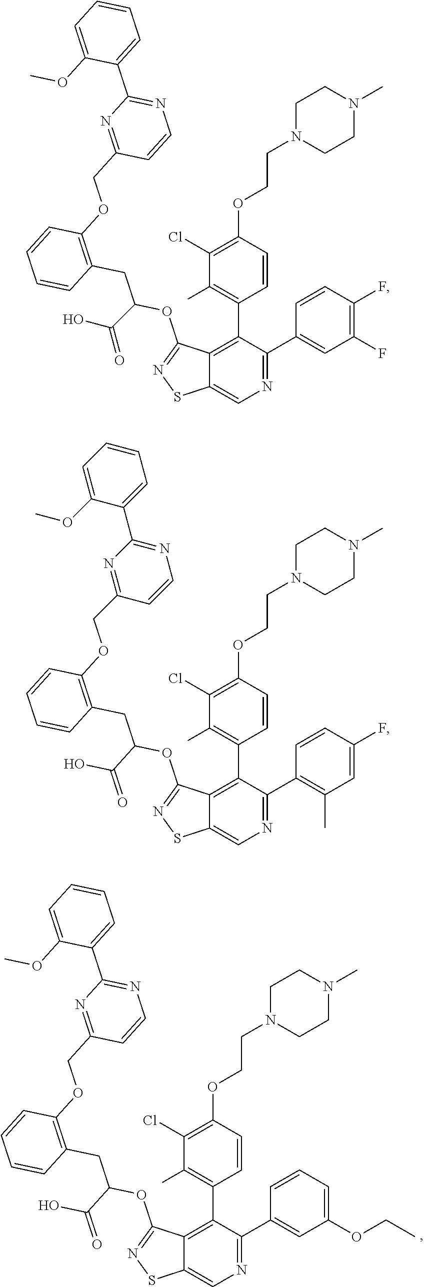 [SMILES: COc1ccccc1-c1nccc(COc2ccccc2CC(Oc2nsc3cnc(-c4ccc(F)c(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(F)cc4C)c(-c4ccc(OCCN5CCN(C)CC5)c(Cl)c4C)c23)C(=O)O)n1 ; CCOc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1]

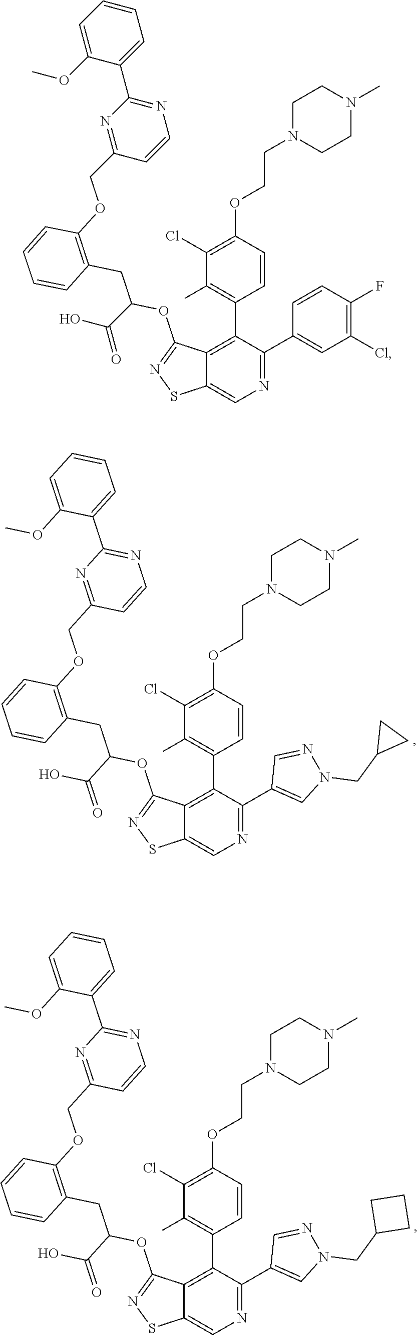 [SMILES: COc1ccccc1-c1nccc(COc2ccccc2CC(Oc2nsc3cnc(-c4ccc(F)c(Cl)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CC5)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CCC5)c4)c(-c4ccc(OCCN5CCN(C)CC5)c(Cl)c4C)c23)C(=O)O)n1]

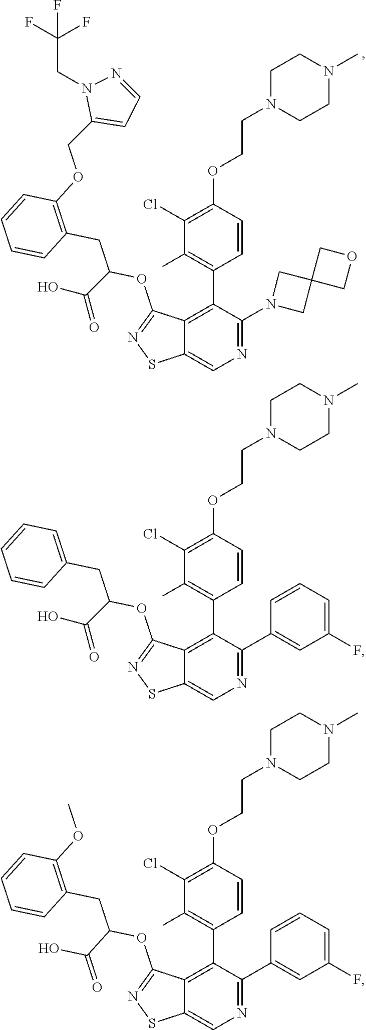 [SMILES: Cc1c(-c2c(N3CC4(COC4)C3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4)C(=O)O)c23)ccc(OCCN2CCN(C)CC2)c1Cl ; COc1ccccc1CC(Oc1nsc2cnc(-c3cccc(F)c3)c(-c3ccc(OCCN4CCN(C)CC4)c(Cl)c3C)c12)C(=O)O]

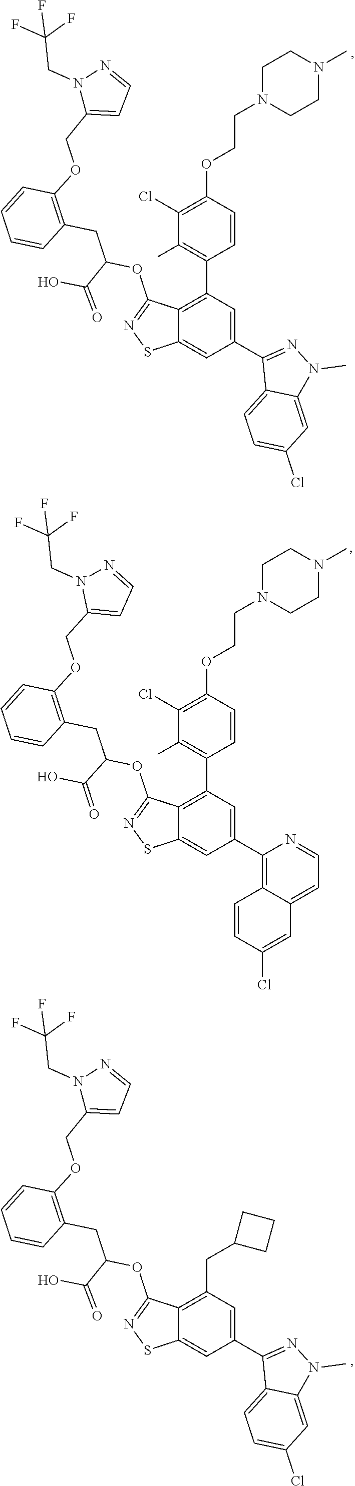

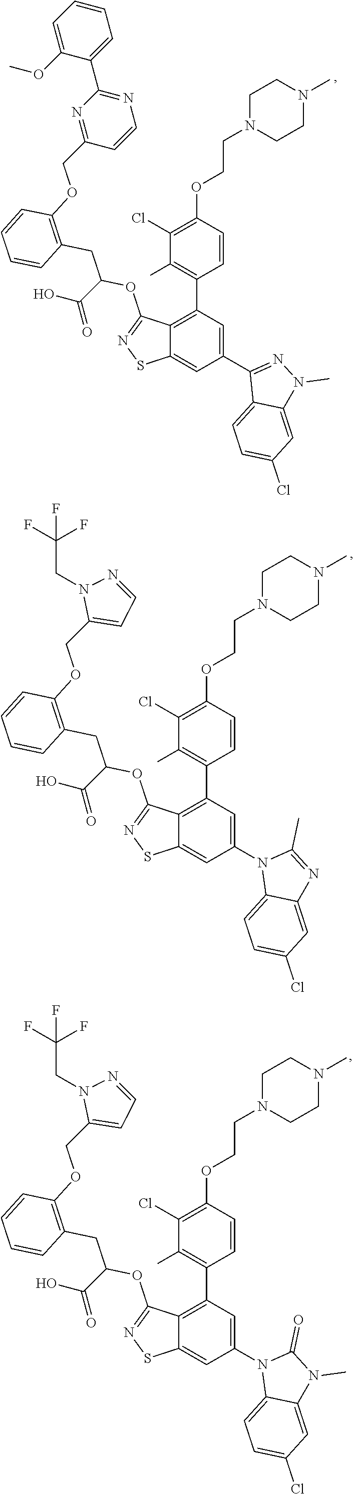 [SMILES: COc1ccccc1-c1nccc(COc2ccccc2CC(Oc2nsc3cc(-c4nn(C)c5cc(Cl)ccc45)cc(-c4ccc(OCCN5CCN(C)CC5)c(Cl)c4C)c23)C(=O)O)n1 ; Cc1c(-c2cc(-n3c(C)nc4cc(Cl)ccc43)cc3snc(OC(Cc4ccccc4OCc4ccnn4CC(F)(F)F)C(=O)O)c23)ccc(OCCN2CCN(C)CC2)c1Cl ; Cc1c(-c2cc(-n3c(=O)n(C)c4cc(Cl)ccc43)cc3snc(OC(Cc4ccccc4OCc4ccnn4CC(F)(F)F)C(=O)O)c23)ccc(OCCN2CCN(C)CC2)c1Cl]

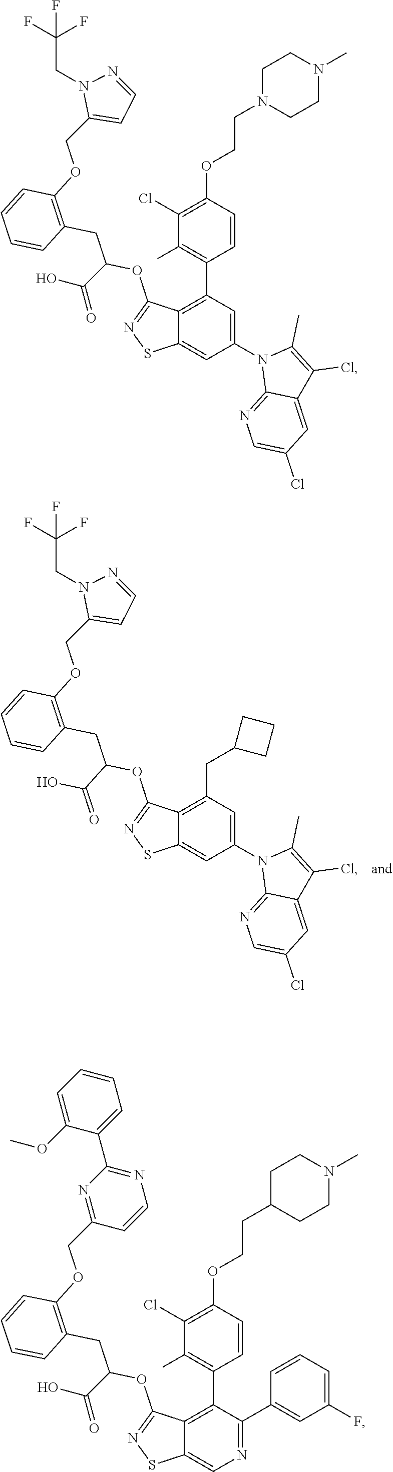 [SMILES: Cc1c(-c2cc(-n3c(C)c(Cl)c4cc(Cl)cnc43)cc3snc(OC(Cc4ccccc4OCc4ccnn4CC(F)(F)F)C(=O)O)c23)ccc(OCCN2CCN(C)CC2)c1Cl ; Cc1c(Cl)c2cc(Cl)cnc2n1-c1cc(CC2CCC2)c2c(OC(Cc3ccccc3OCc3ccnn3CC(F)(F)F)C(=O)O)nsc2c1 ; COc1ccccc1-c1nccc(COc2ccccc2CC(Oc2nsc3cnc(-c4cccc(F)c4)c(-c4ccc(OCCC5CCN(C)CC5)c(Cl)c4C)c23)C(=O)O)n1]

pharmaceutically acceptable salt thereof.

In some aspects, the invention relates to a compound of Formula II having a structure selected from:

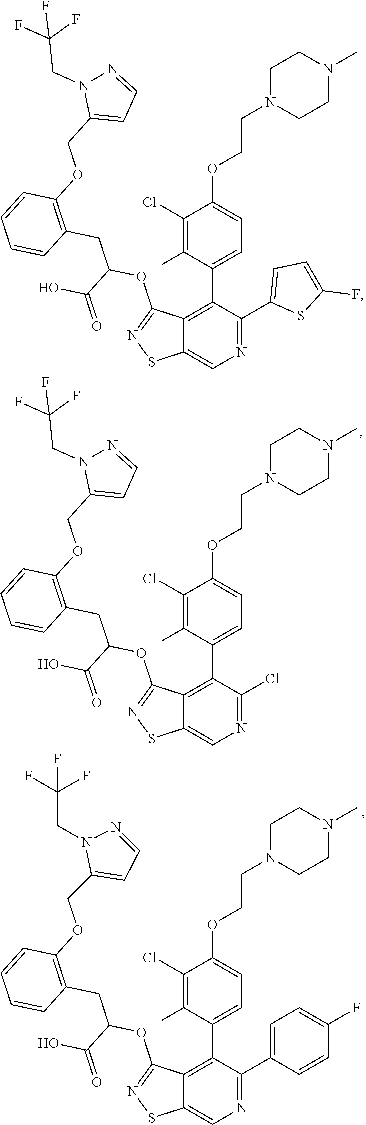 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl]

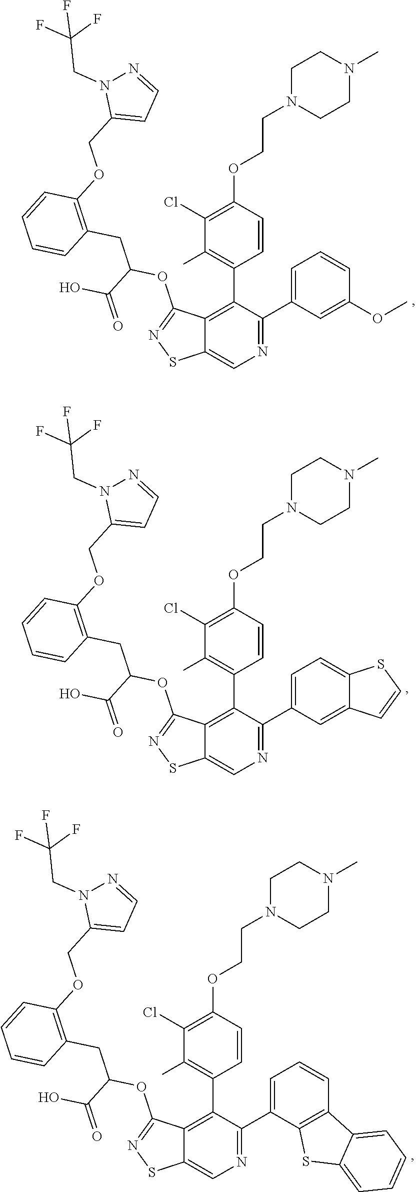 [SMILES: COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3ccc4sccc4c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3sc3ccccc34)ncc3snc(OC(Cc4ccccc4OCc4ccnn4CC(F)(F)F)C(=O)O)c23)ccc(OCCN2CCN(C)CC2)c1Cl]

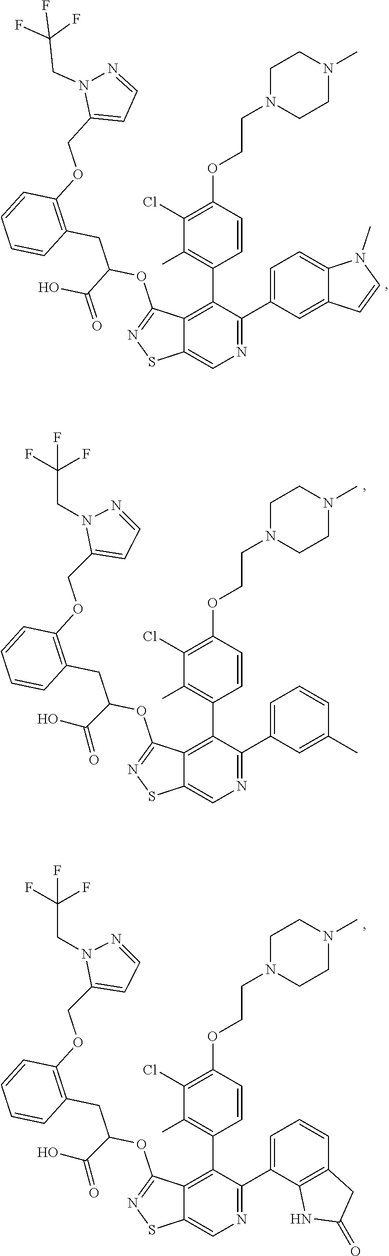 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl]

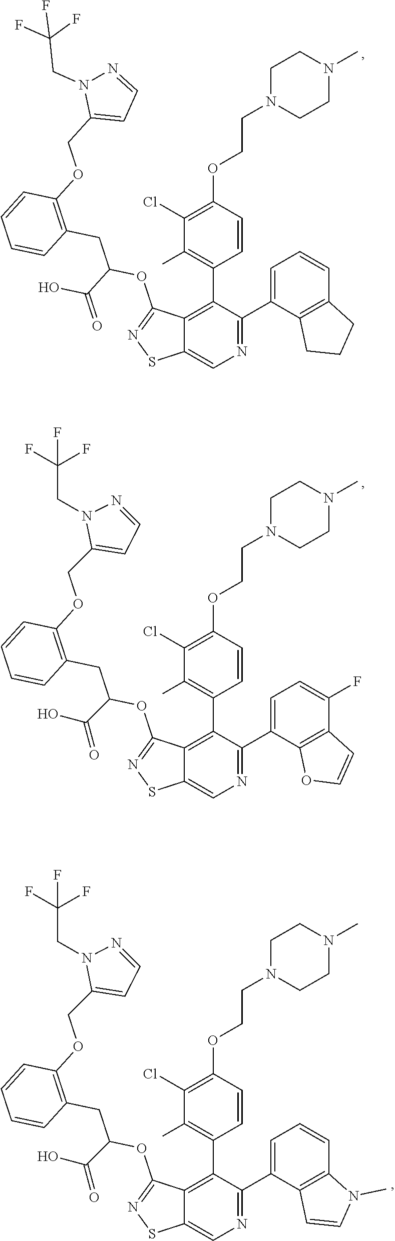 [SMILES: Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl]

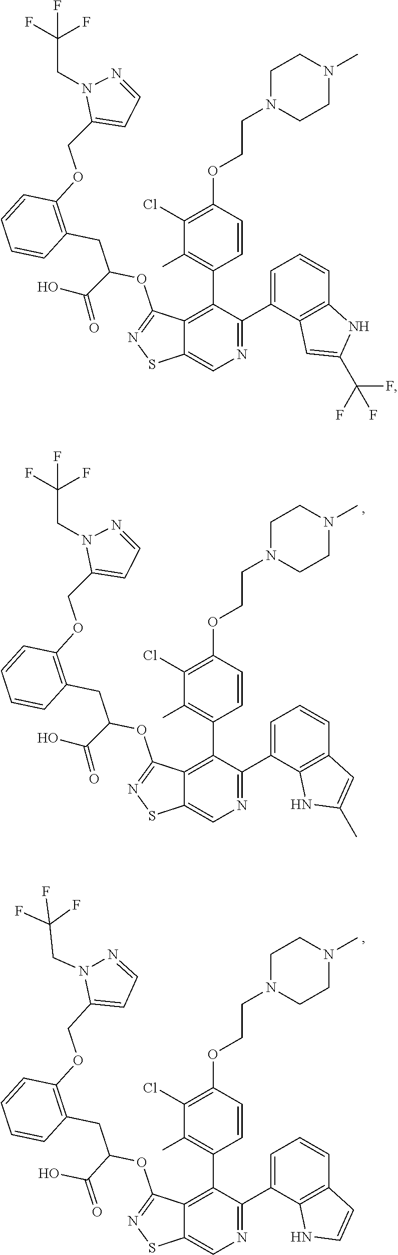 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1 ; Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl]

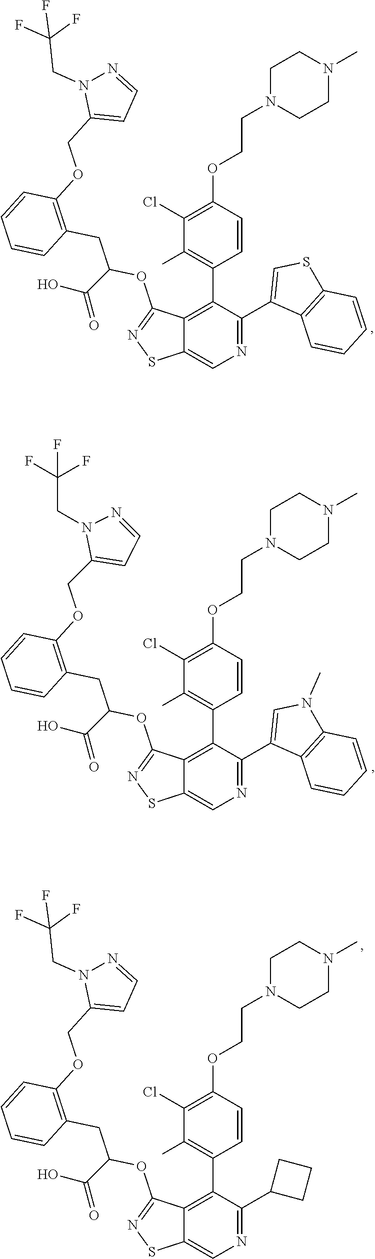 [SMILES: Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl]

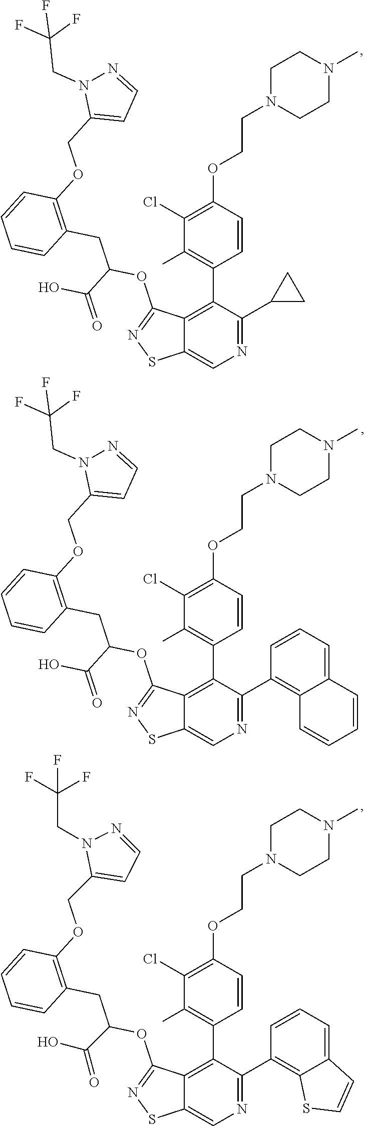 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl]

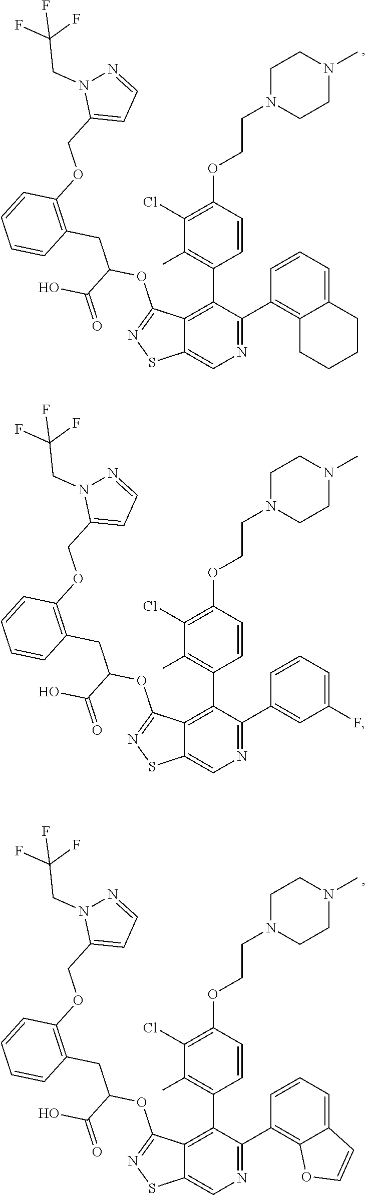 [SMILES: Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl]

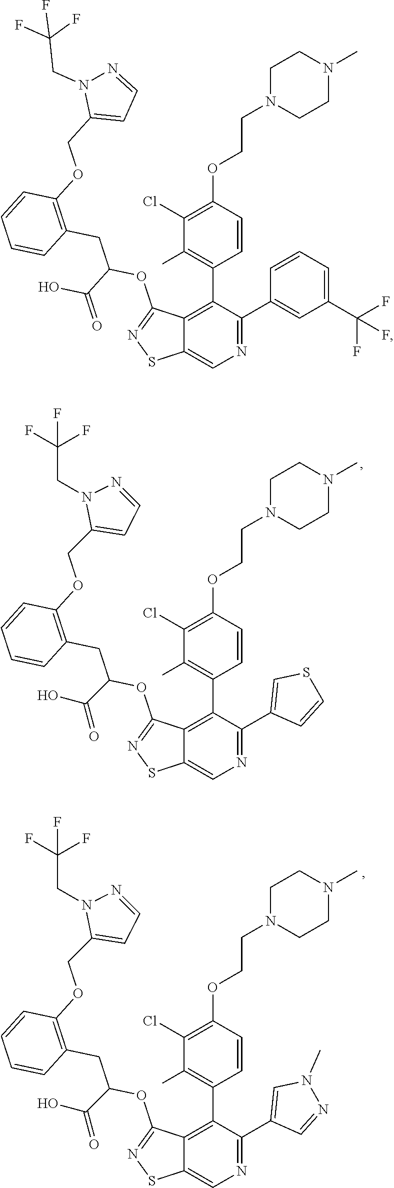 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl]

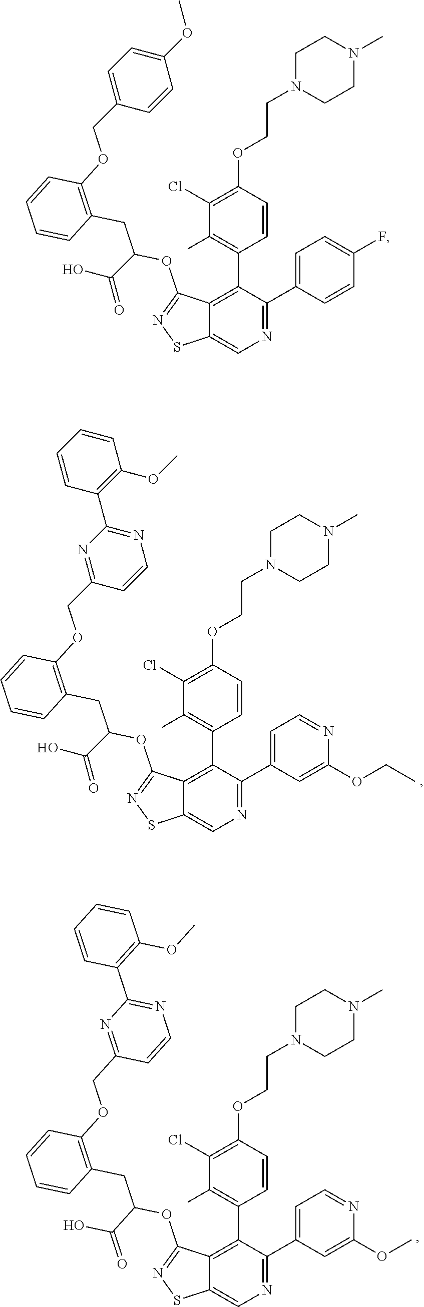 [SMILES: COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1 ; CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1]

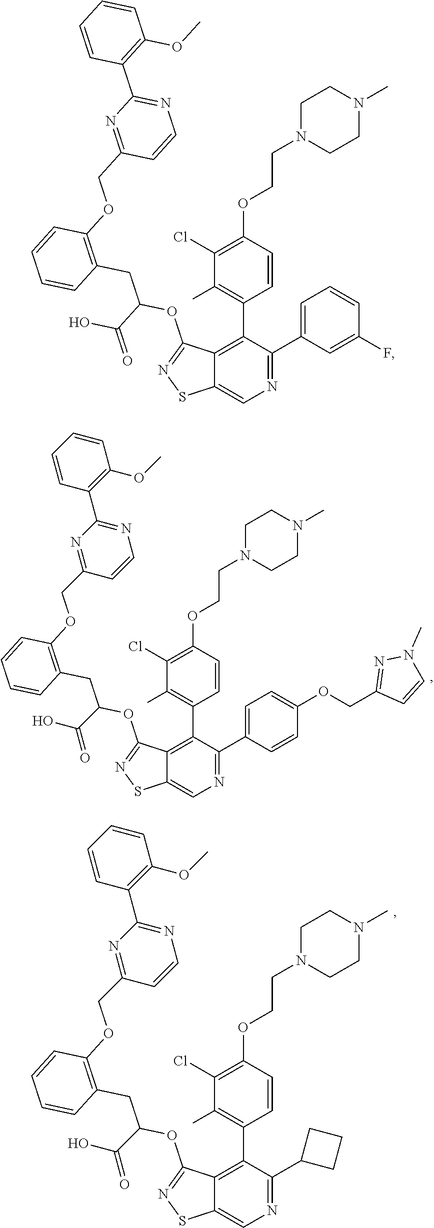

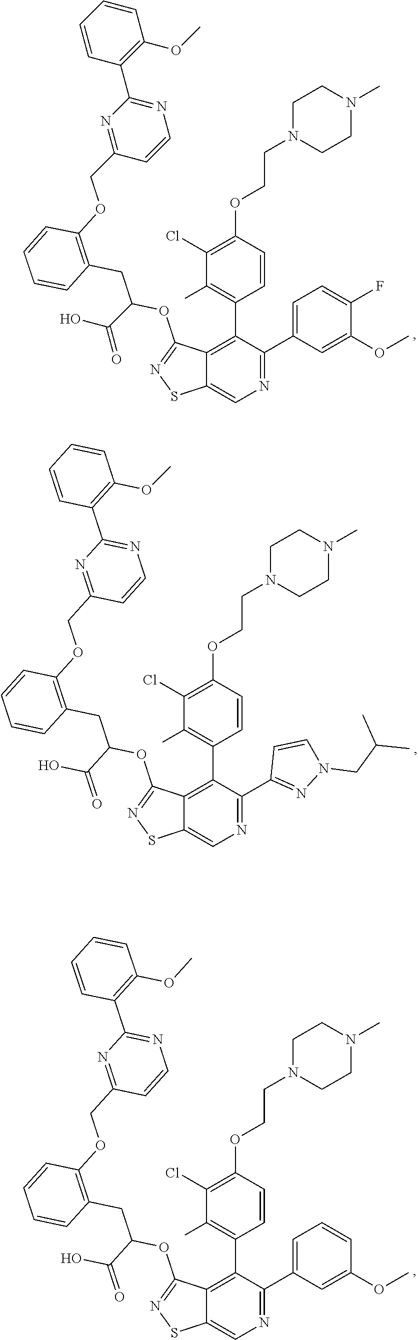 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F ; COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1]

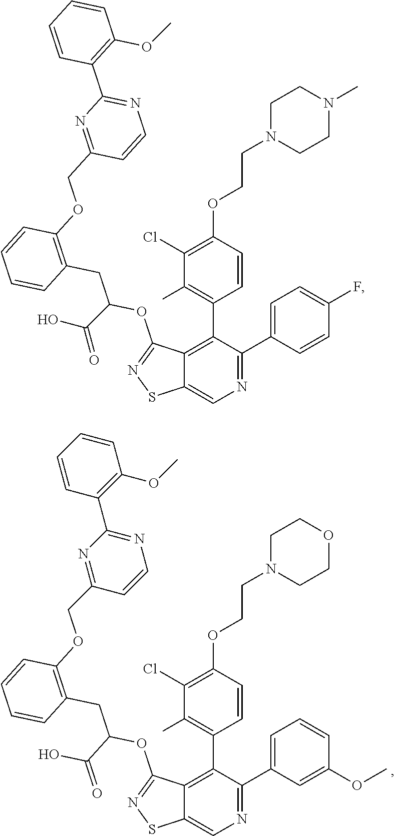 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1]

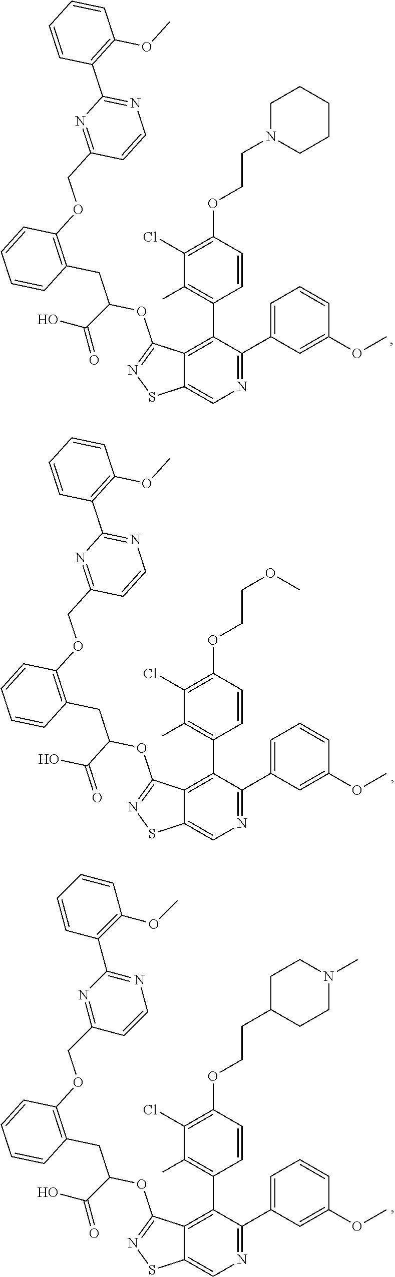 [SMILES: COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl ; COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1]

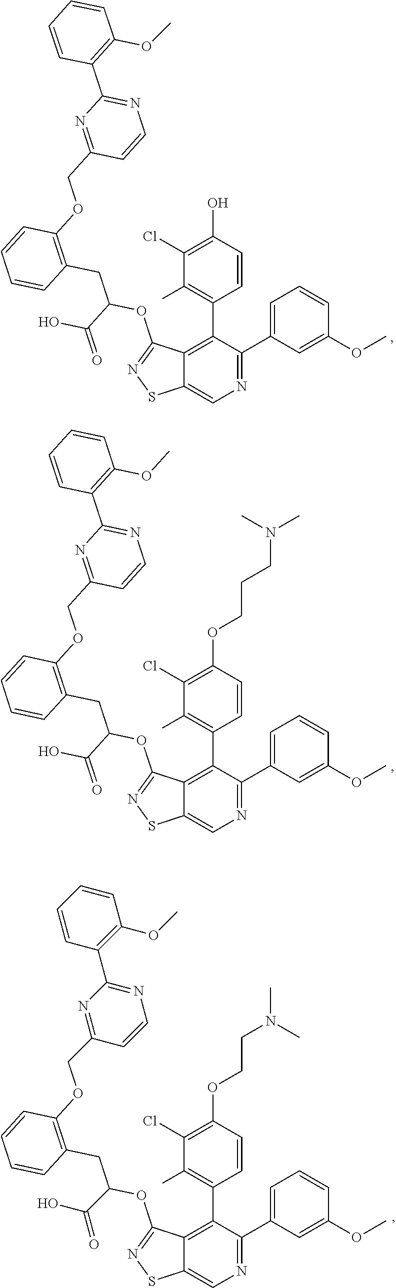 [SMILES: COc1cccc(-c2ncc3snc(OC(Cc4ccccc4OCc4ccnc(-c5ccccc5OC)n4)C(=O)O)c3c2-c2ccc(O)c(Cl)c2C)c1 ; COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1]

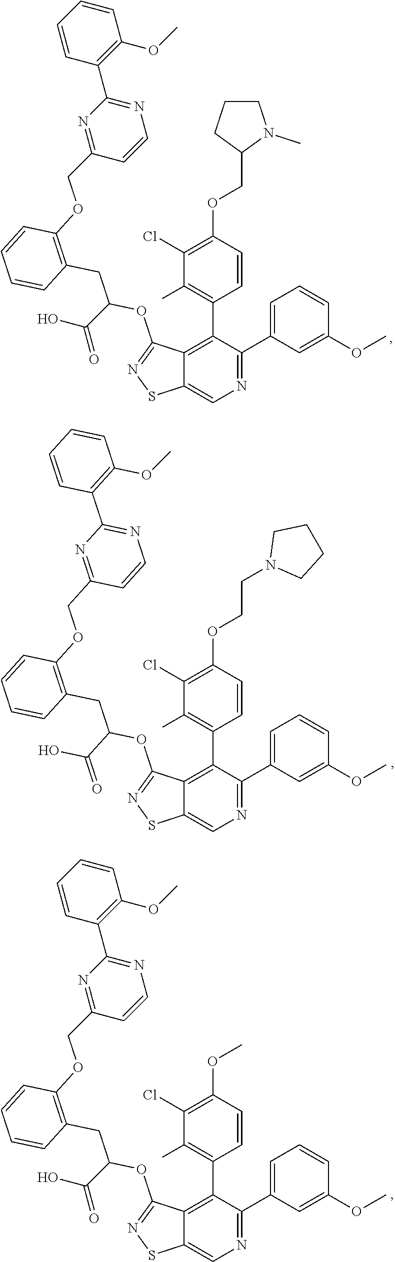 [SMILES: COc1cccc(-c2ncc3snc(OC(Cc4ccccc4OCc4ccnc(-c5ccccc5OC)n4)C(=O)O)c3c2-c2ccc(OCC3CCCN3C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OC)c(Cl)c2C)c1]

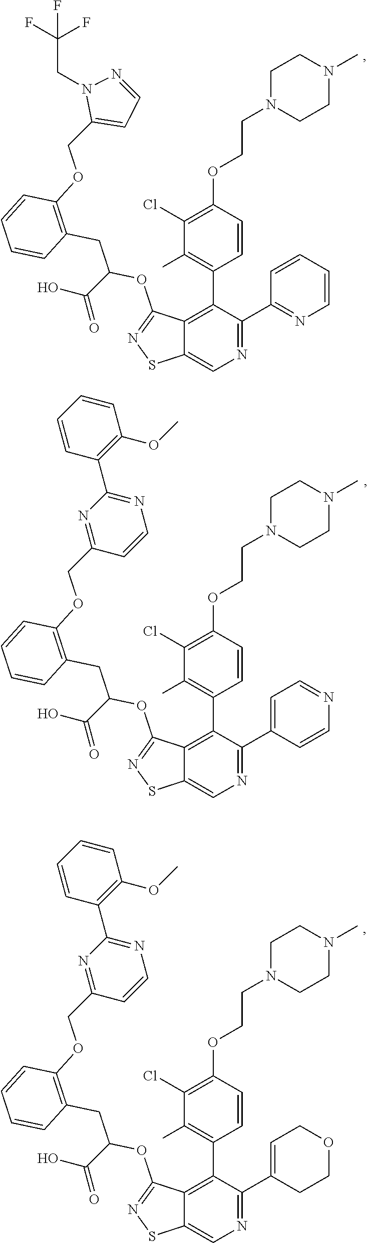 [SMILES: Cc1c(-c2c(-c3ccccn3)ncc3snc(OC(Cc4ccccc4OCc4ccnn4CC(F)(F)F)C(=O)O)c23)ccc(OCCN2CCN(C)CC2)c1Cl ; COc1ccccc1-c1nccc(COc2ccccc2CC(Oc2nsc3cnc(-c4ccncc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4=CCOCC4)c(-c4ccc(OCCN5CCN(C)CC5)c(Cl)c4C)c23)C(=O)O)n1]

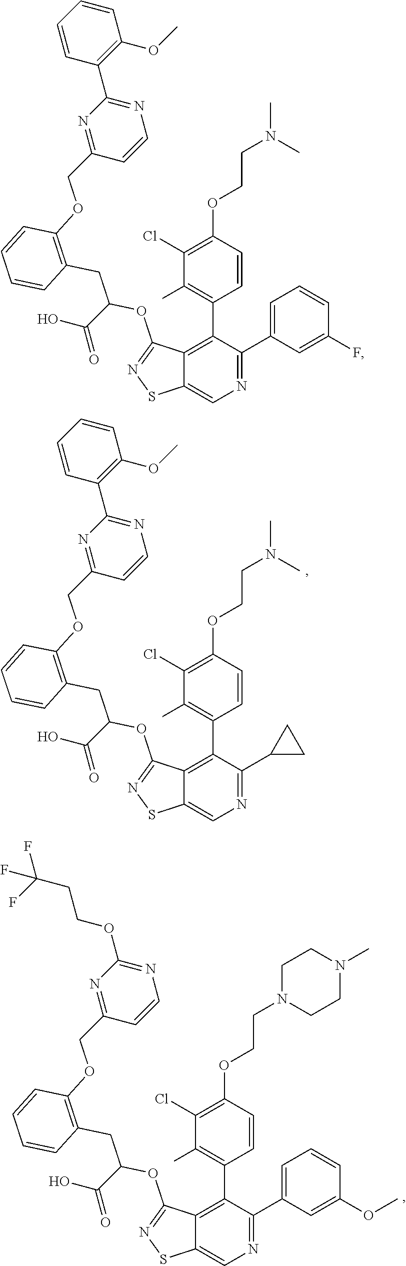 [SMILES: COc1ccccc1-c1nccc(COc2ccccc2CC(Oc2nsc3cnc(-c4cccc(F)c4)c(-c4ccc(OCCN(C)C)c(Cl)c4C)c23)C(=O)O)n1 ; COc1ccccc1-c1nccc(COc2ccccc2CC(Oc2nsc3cnc(C4CC4)c(-c4ccc(OCCN(C)C)c(Cl)c4C)c23)C(=O)O)n1 ; COc1cccc(-c2ncc3snc(OC(Cc4ccccc4OCc4ccnc(OCCC(F)(F)F)n4)C(=O)O)c3c2-c2ccc(OCCN3CCN(C)CC3)c(Cl)c2C)c1]

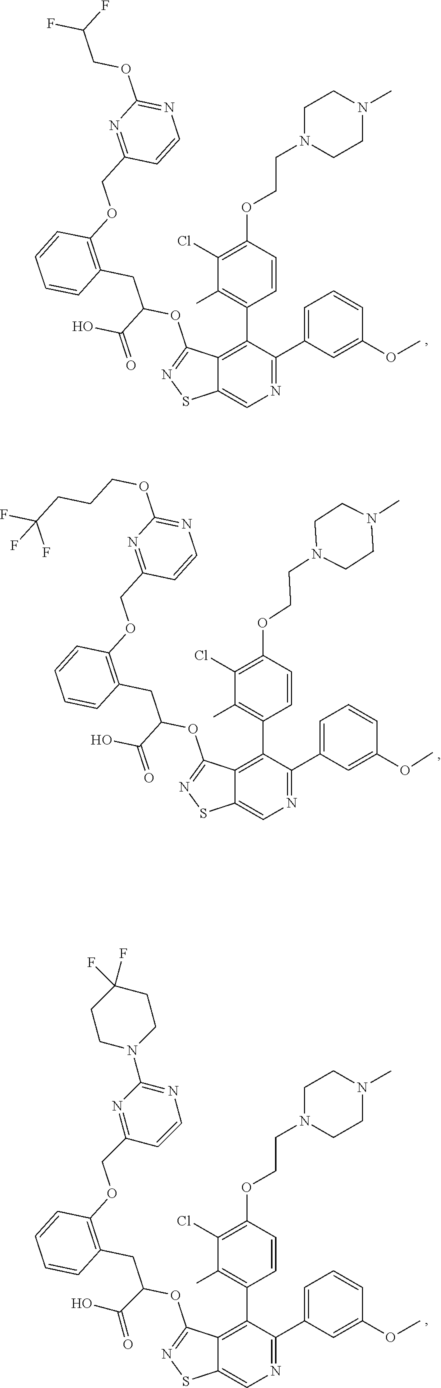 [SMILES: COc1cccc(-c2ncc3snc(OC(Cc4ccccc4OCc4ccnc(OCC(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(OCCCC(F)(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(F)(F)CC5)n4)C(=O)O)c3c2-c2ccc(OCCN3CCN(C)CC3)c(Cl)c2C)c1]

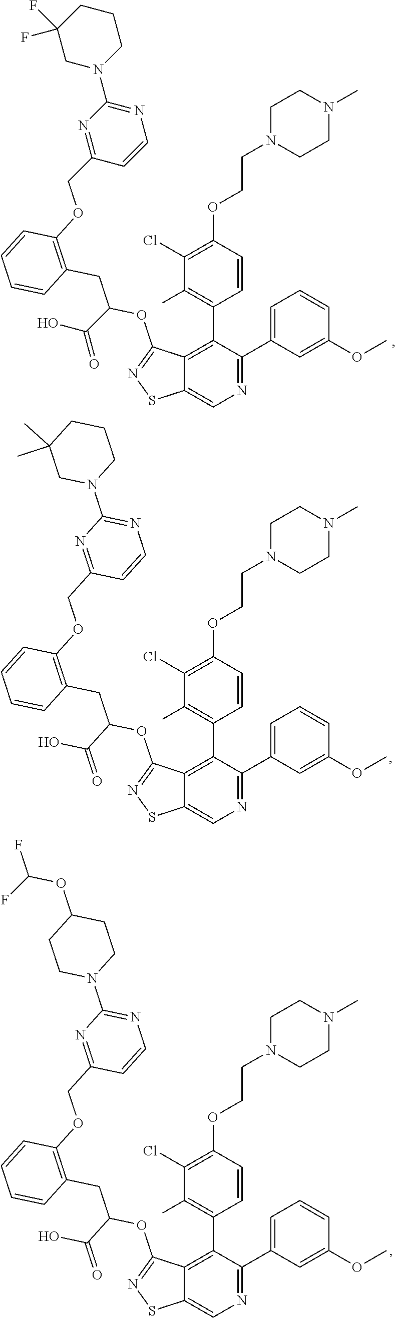 [SMILES: COc1cccc(-c2ncc3snc(OC(Cc4ccccc4OCc4ccnc(N5CCCC(F)(F)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCCC(C)(C)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(OC(F)F)CC5)n4)C(=O)O)c3c2-c2ccc(OCCN3CCN(C)CC3)c(Cl)c2C)c1]

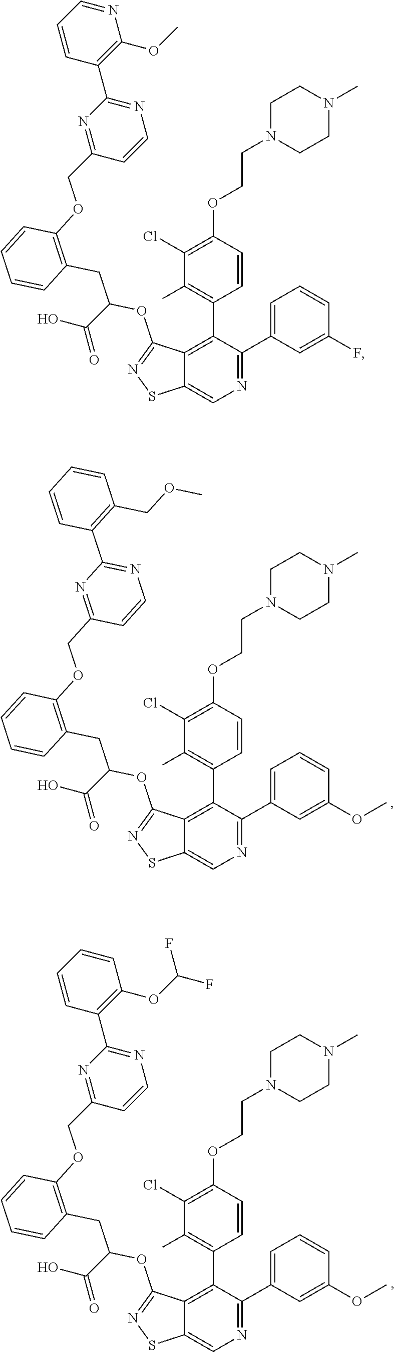 [SMILES: COc1ncccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1 ; COCc1ccccc1-c1nccc(COc2ccccc2CC(Oc2nsc3cnc(-c4cccc(OC)c4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC(F)F)n4)C(=O)O)c3c2-c2ccc(OCCN3CCN(C)CC3)c(Cl)c2C)c1]

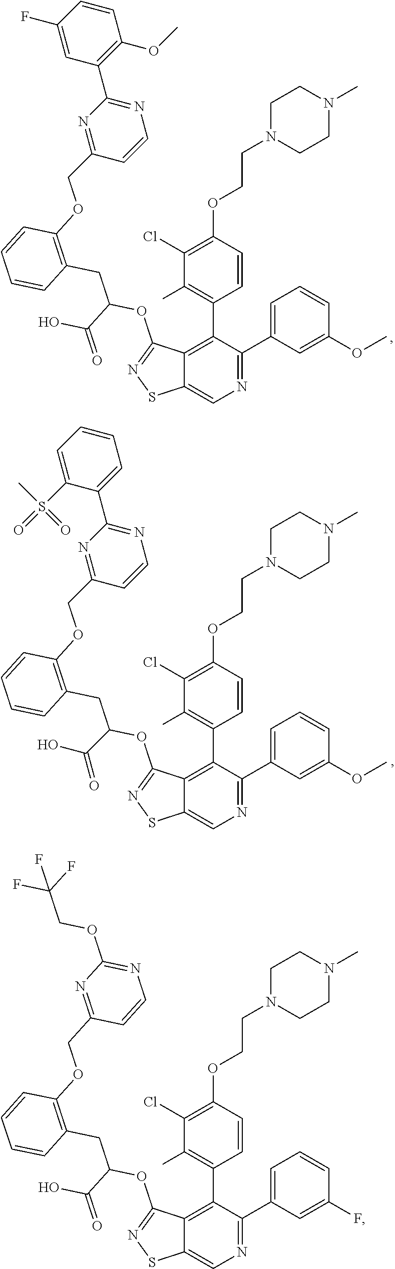 [SMILES: COc1cccc(-c2ncc3snc(OC(Cc4ccccc4OCc4ccnc(-c5cc(F)ccc5OC)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(-c5ccccc5S(C)(=O)=O)n4)C(=O)O)c3c2-c2ccc(OCCN3CCN(C)CC3)c(Cl)c2C)c1 ; Cc1c(-c2c(-c3cccc(F)c3)ncc3snc(OC(Cc4ccccc4OCc4ccnc(OCC(F)(F)F)n4)C(=O)O)c23)ccc(OCCN2CCN(C)CC2)c1Cl]

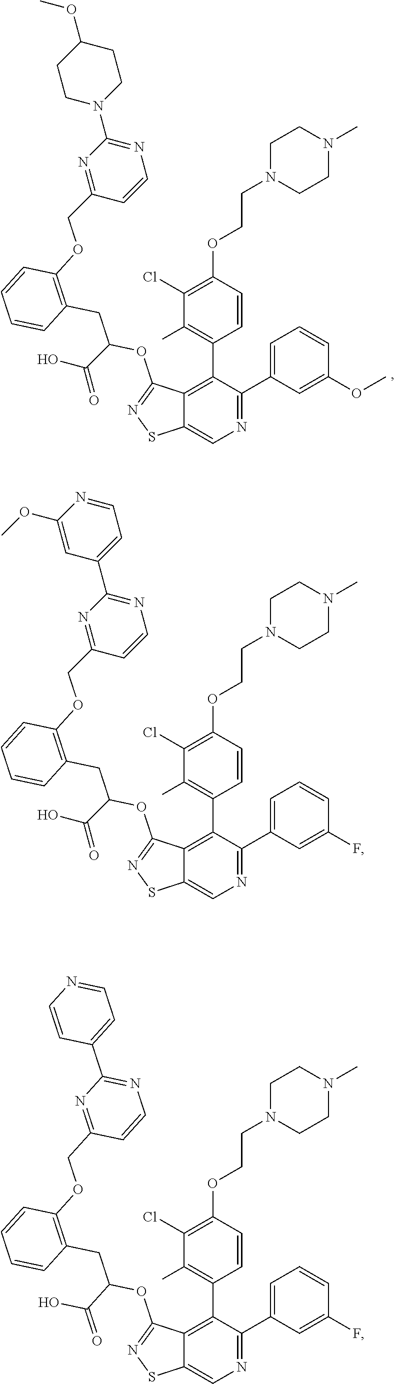 [SMILES: COc1cccc(-c2ncc3snc(OC(Cc4ccccc4OCc4ccnc(N5CCC(OC)CC5)n4)C(=O)O)c3c2-c2ccc(OCCN3CCN(C)CC3)c(Cl)c2C)c1 ; COc1cc(-c2nccc(COc3ccccc3CC(Oc3nsc4cnc(-c5cccc(F)c5)c(-c5ccc(OCCN6CCN(C)CC6)c(Cl)c5C)c34)C(=O)O)n2)ccn1 ; Cc1c(-c2c(-c3cccc(F)c3)ncc3snc(OC(Cc4ccccc4OCc4ccnc(-c5ccncc5)n4)C(=O)O)c23)ccc(OCCN2CCN(C)CC2)c1Cl]

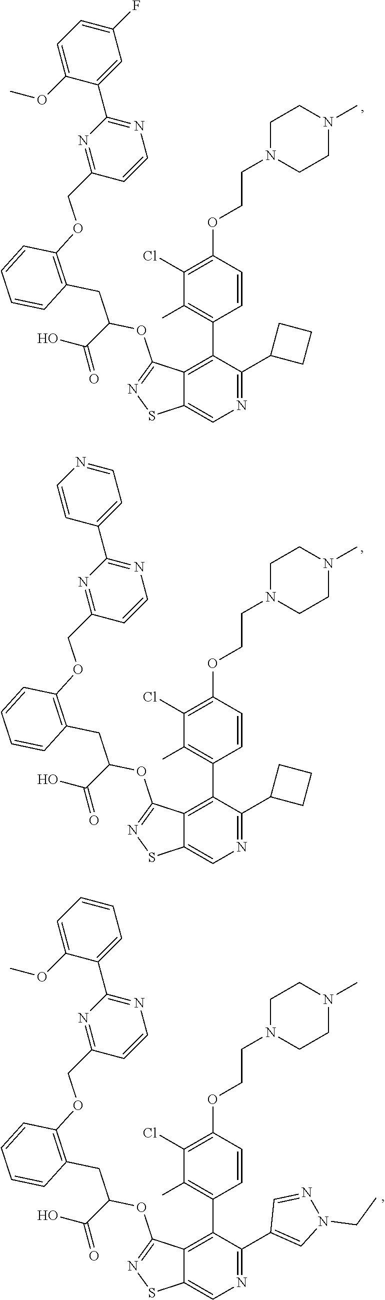 [SMILES: COc1ccc(F)cc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5ccncc5)n4)C(=O)O)c23)ccc(OCCN2CCN(C)CC2)c1Cl ; CCn1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)cn1]

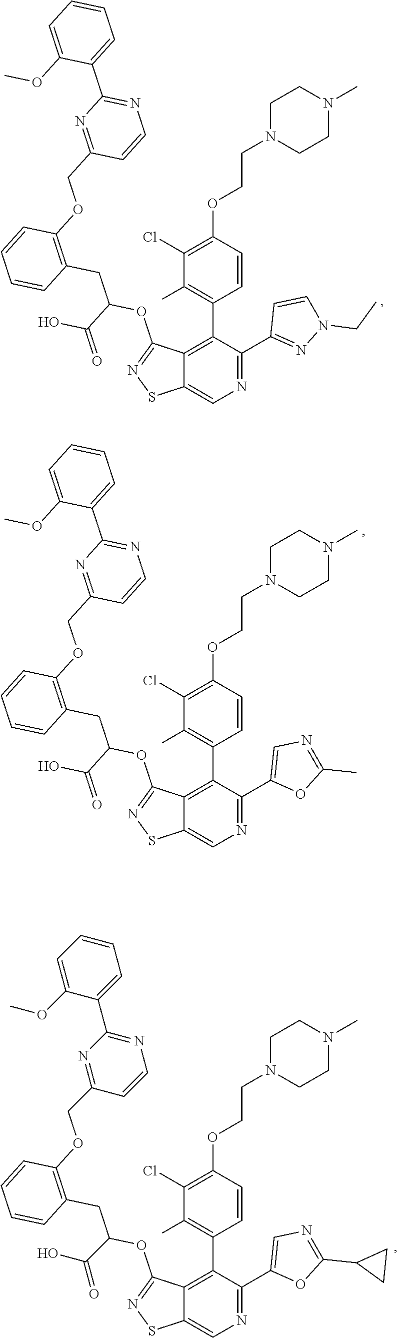 [SMILES: CCn1ccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)n1 ; COc1ccccc1-c1nccc(COc2ccccc2CC(Oc2nsc3cnc(-c4cnc(C)o4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnc(C5CC5)o4)c(-c4ccc(OCCN5CCN(C)CC5)c(Cl)c4C)c23)C(=O)O)n1]

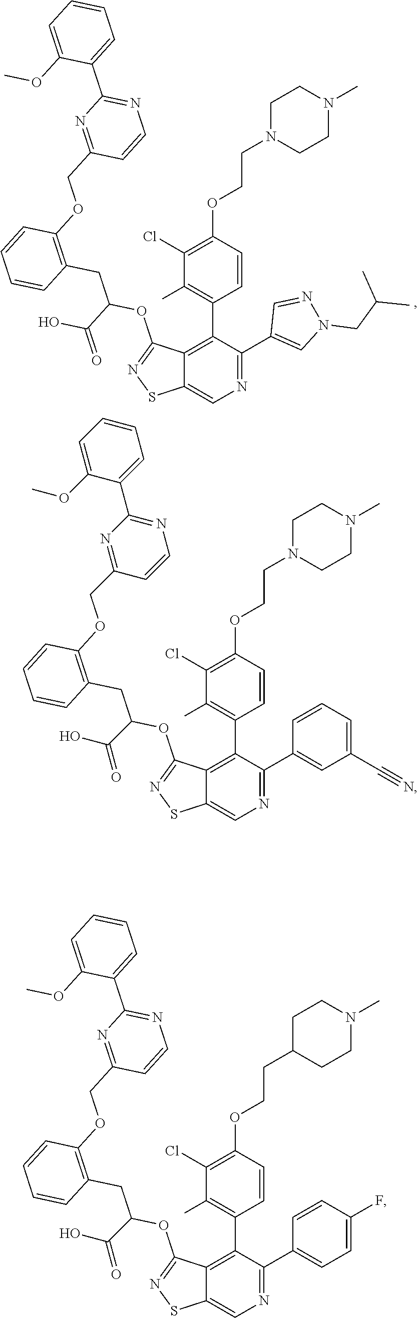

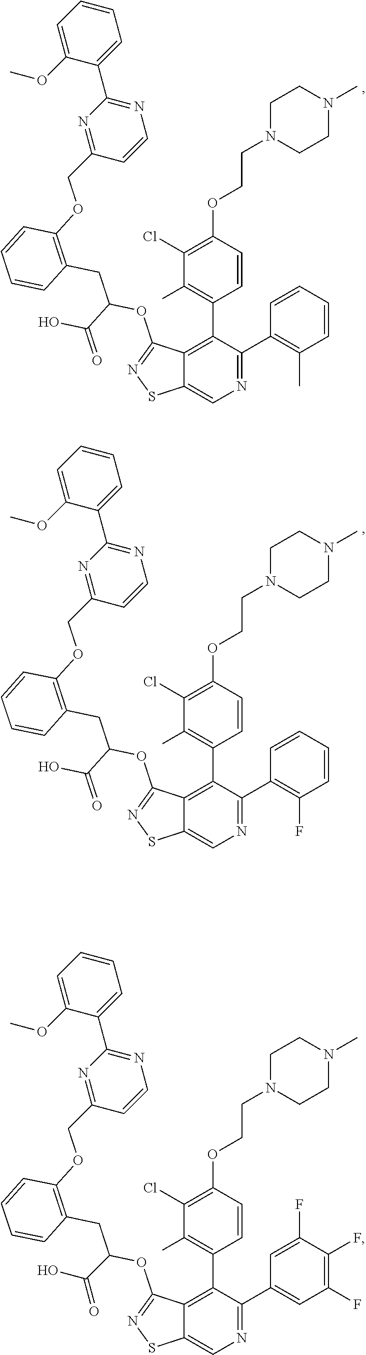 [SMILES: COc1ccccc1-c1nccc(COc2ccccc2CC(Oc2nsc3cnc(-c4ccccc4C)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccccc4F)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cc(F)c(F)c(F)c4)c(-c4ccc(OCCN5CCN(C)CC5)c(Cl)c4C)c23)C(=O)O)n1]

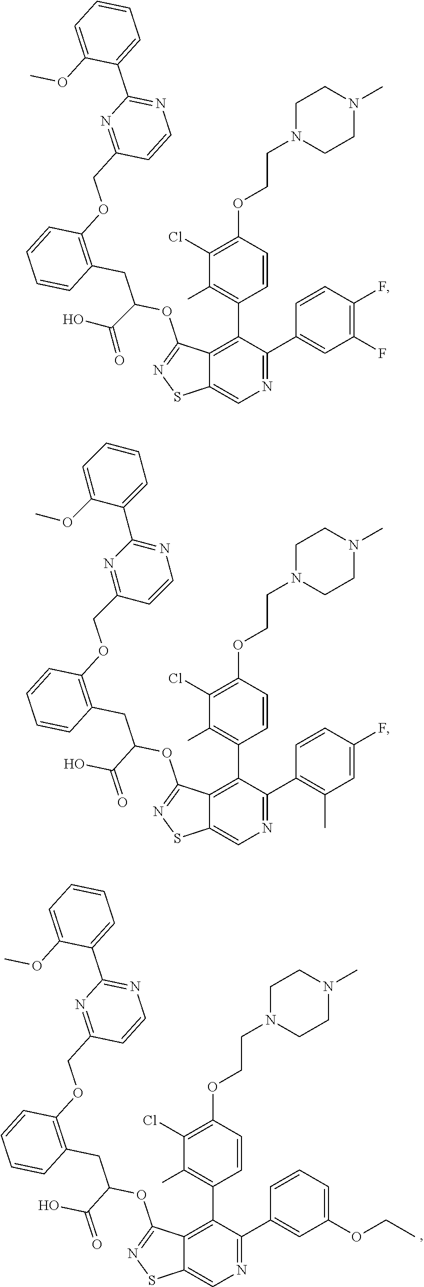 [SMILES: COc1ccccc1-c1nccc(COc2ccccc2CC(Oc2nsc3cnc(-c4ccc(F)c(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(F)cc4C)c(-c4ccc(OCCN5CCN(C)CC5)c(Cl)c4C)c23)C(=O)O)n1 ; CCOc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1]

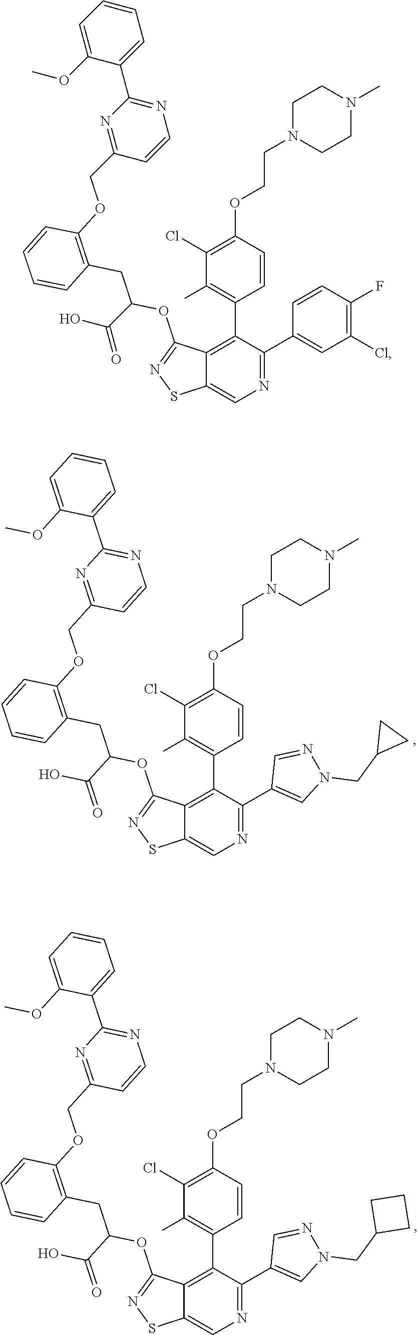 [SMILES: COc1ccccc1-c1nccc(COc2ccccc2CC(Oc2nsc3cnc(-c4ccc(F)c(Cl)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CC5)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CCC5)c4)c(-c4ccc(OCCN5CCN(C)CC5)c(Cl)c4C)c23)C(=O)O)n1]

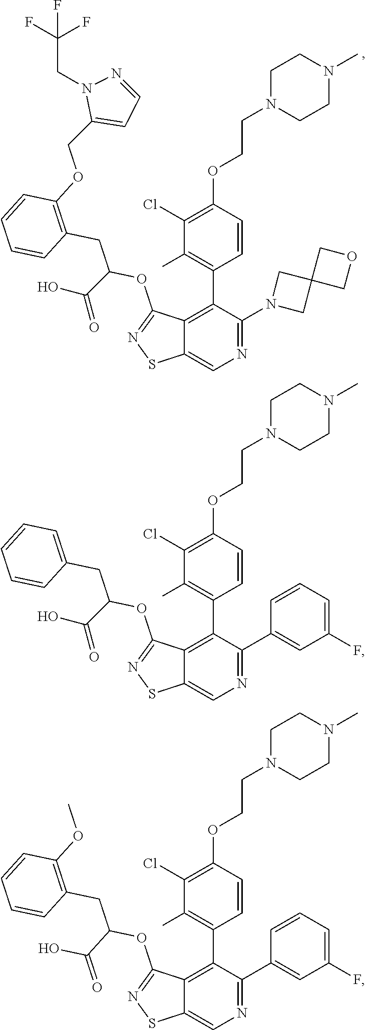 [SMILES: Cc1c(-c2c(N3CC4(COC4)C3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4)C(=O)O)c23)ccc(OCCN2CCN(C)CC2)c1Cl ; COc1ccccc1CC(Oc1nsc2cnc(-c3cccc(F)c3)c(-c3ccc(OCCN4CCN(C)CC4)c(Cl)c3C)c12)C(=O)O]

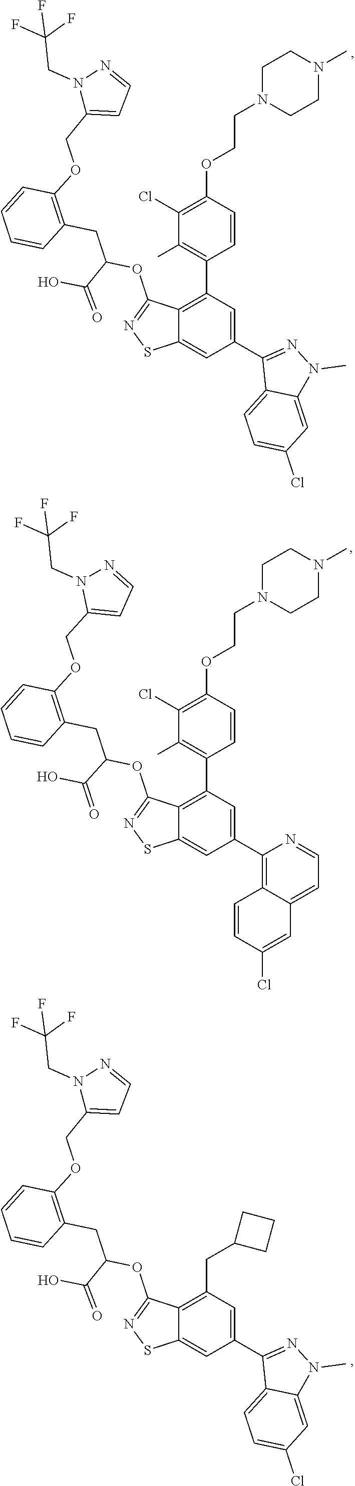

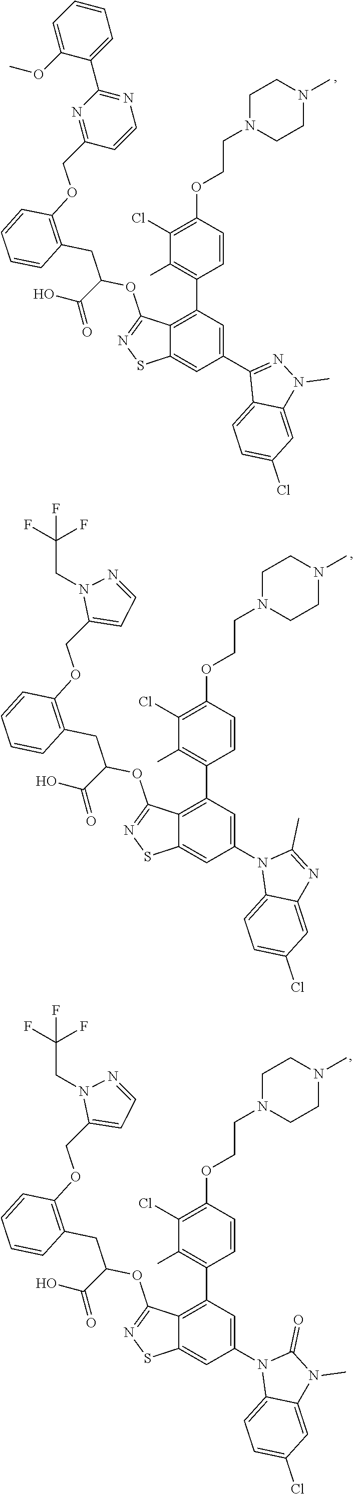 [SMILES: COc1ccccc1-c1nccc(COc2ccccc2CC(Oc2nsc3cc(-c4nn(C)c5cc(Cl)ccc45)cc(-c4ccc(OCCN5CCN(C)CC5)c(Cl)c4C)c23)C(=O)O)n1 ; Cc1c(-c2cc(-n3c(C)nc4cc(Cl)ccc43)cc3snc(OC(Cc4ccccc4OCc4ccnn4CC(F)(F)F)C(=O)O)c23)ccc(OCCN2CCN(C)CC2)c1Cl ; Cc1c(-c2cc(-n3c(=O)n(C)c4cc(Cl)ccc43)cc3snc(OC(Cc4ccccc4OCc4ccnn4CC(F)(F)F)C(=O)O)c23)ccc(OCCN2CCN(C)CC2)c1Cl]

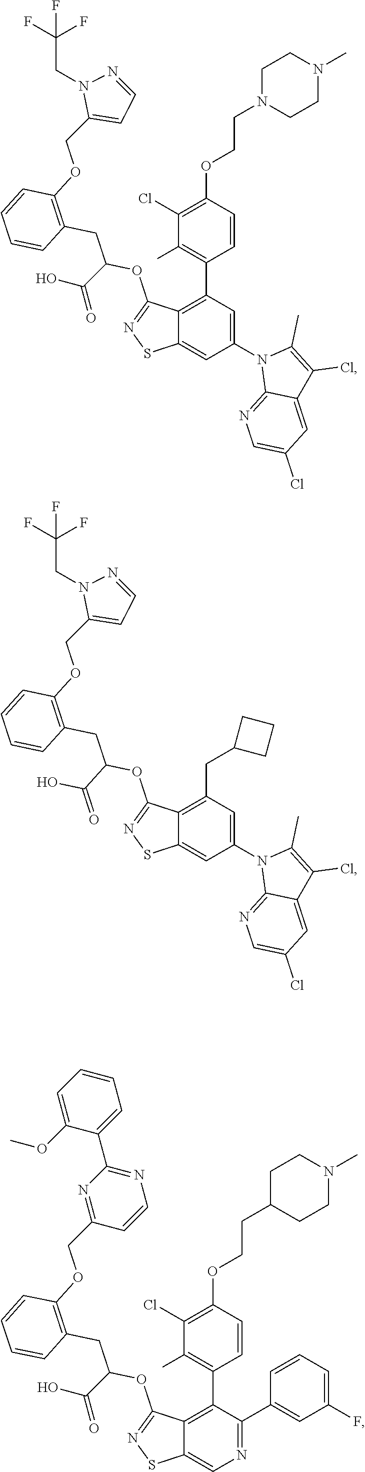 [SMILES: Cc1c(-c2cc(-n3c(C)c(Cl)c4cc(Cl)cnc43)cc3snc(OC(Cc4ccccc4OCc4ccnn4CC(F)(F)F)C(=O)O)c23)ccc(OCCN2CCN(C)CC2)c1Cl ; Cc1c(Cl)c2cc(Cl)cnc2n1-c1cc(CC2CCC2)c2c(OC(Cc3ccccc3OCc3ccnn3CC(F)(F)F)C(=O)O)nsc2c1 ; COc1ccccc1-c1nccc(COc2ccccc2CC(Oc2nsc3cnc(-c4cccc(F)c4)c(-c4ccc(OCCC5CCN(C)CC5)c(Cl)c4C)c23)C(=O)O)n1]

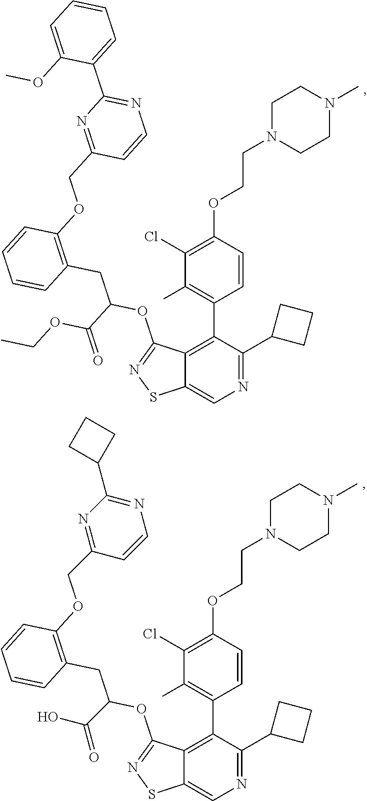 [SMILES: CCOC(=O)C(Cc1ccccc1OCc1ccnc(-c2ccccc2OC)n1)Oc1nsc2cnc(C3CCC3)c(-c3ccc(OCCN4CCN(C)CC4)c(Cl)c3C)c12 ; Cc1c(-c2c(C3CCC3)ncc3snc(OC(Cc4ccccc4OCc4ccnc(C5CCC5)n4)C(=O)O)c23)ccc(OCCN2CCN(C)CC2)c1Cl]

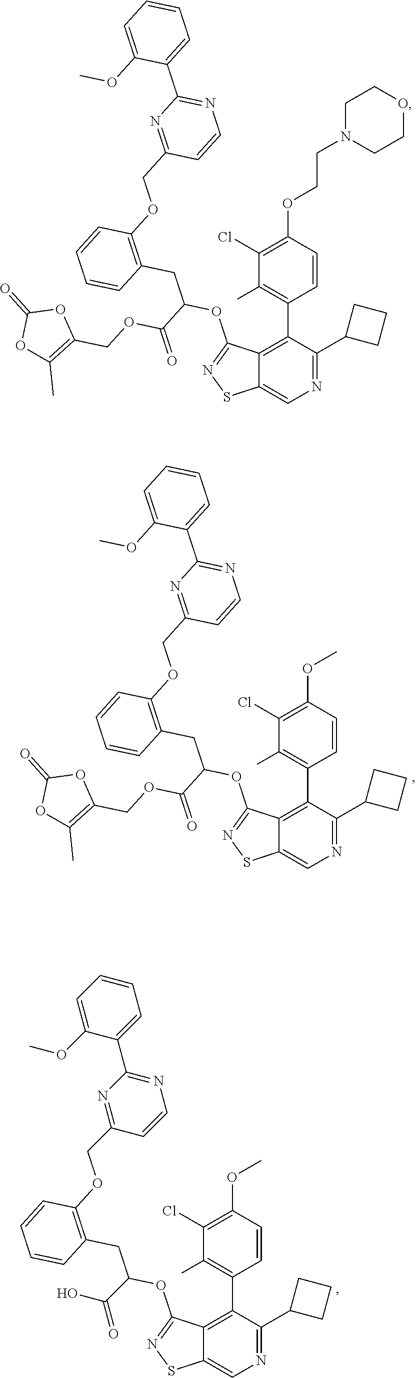 [SMILES: COc1ccccc1-c1nccc(COc2ccccc2CC(Oc2nsc3cnc(C4CCC4)c(-c4ccc(OCCN5CCOCC5)c(Cl)c4C)c23)C(=O)OCc2oc(=O)oc2C)n1 ; COc1ccccc1-c1nccc(COc2ccccc2CC(Oc2nsc3cnc(C4CCC4)c(-c4ccc(OC)c(Cl)c4C)c23)C(=O)OCc2oc(=O)oc2C)n1 ; COc1ccccc1-c1nccc(COc2ccccc2CC(Oc2nsc3cnc(C4CCC4)c(-c4ccc(OC)c(Cl)c4C)c23)C(=O)O)n1]

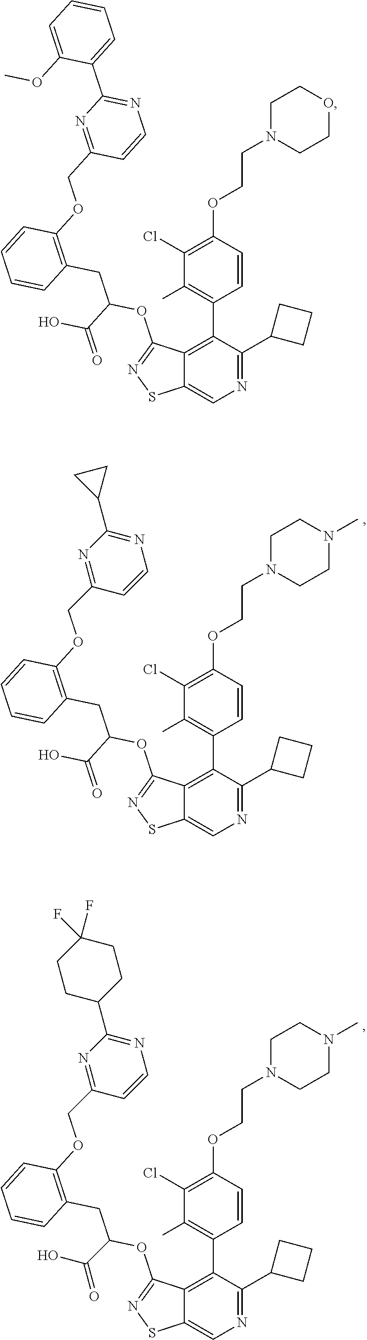 [SMILES: COc1ccccc1-c1nccc(COc2ccccc2CC(Oc2nsc3cnc(C4CCC4)c(-c4ccc(OCCN5CCOCC5)c(Cl)c4C)c23)C(=O)O)n1 ; Cc1c(-c2c(C3CCC3)ncc3snc(OC(Cc4ccccc4OCc4ccnc(C5CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCC(F)(F)CC5)n4)C(=O)O)c23)ccc(OCCN2CCN(C)CC2)c1Cl]

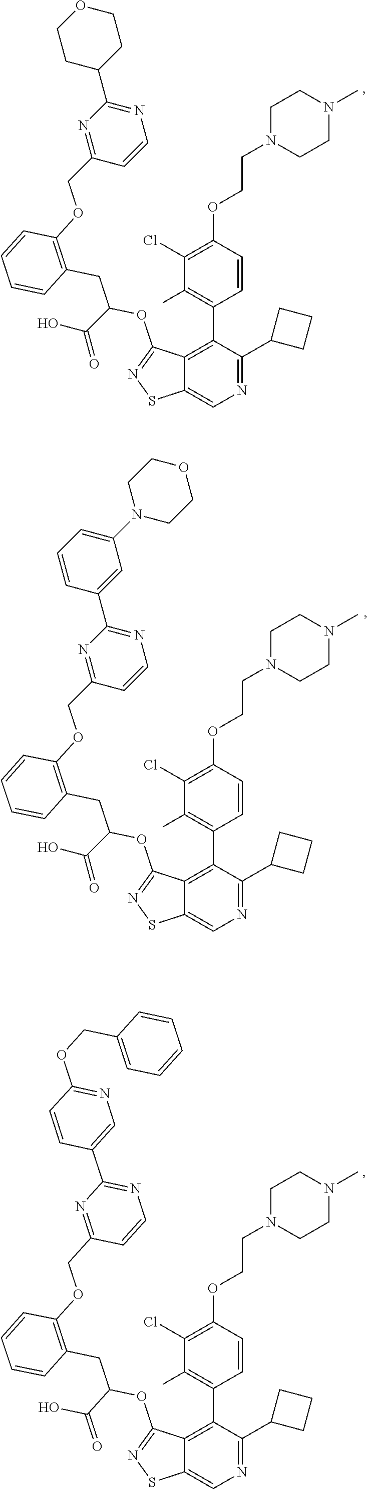 [SMILES: Cc1c(-c2c(C3CCC3)ncc3snc(OC(Cc4ccccc4OCc4ccnc(C5CCOCC5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5cccc(N6CCOCC6)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(OCc6ccccc6)nc5)n4)C(=O)O)c23)ccc(OCCN2CCN(C)CC2)c1Cl]

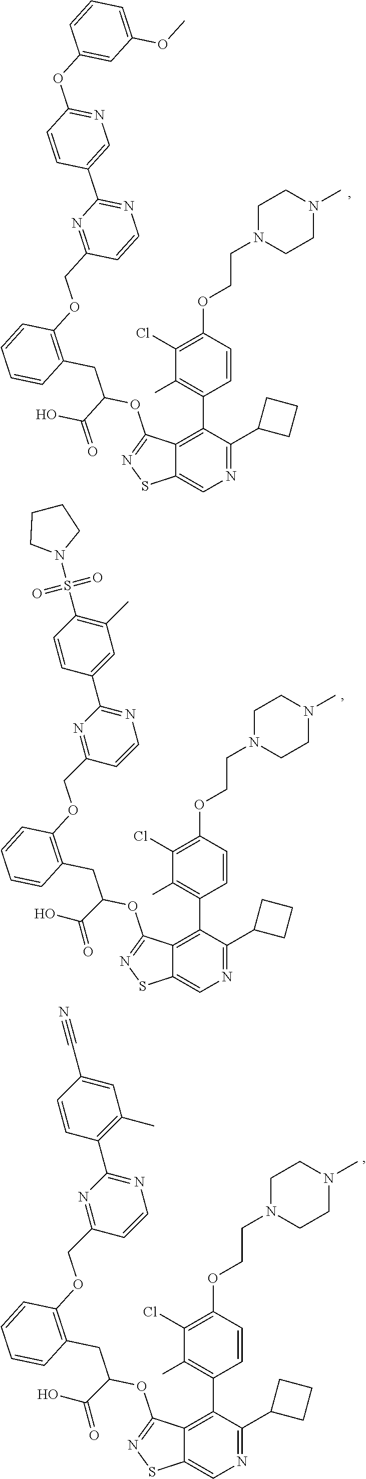 [SMILES: COc1cccc(Oc2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cn2)c1 ; Cc1cc(-c2nccc(COc3ccccc3CC(Oc3nsc4cnc(C5CCC5)c(-c5ccc(OCCN6CCN(C)CC6)c(Cl)c5C)c34)C(=O)O)n2)ccc1S(=O)(=O)N1CCCC1 ; Cc1cc(C#N)ccc1-c1nccc(COc2ccccc2CC(Oc2nsc3cnc(C4CCC4)c(-c4ccc(OCCN5CCN(C)CC5)c(Cl)c4C)c23)C(=O)O)n1]

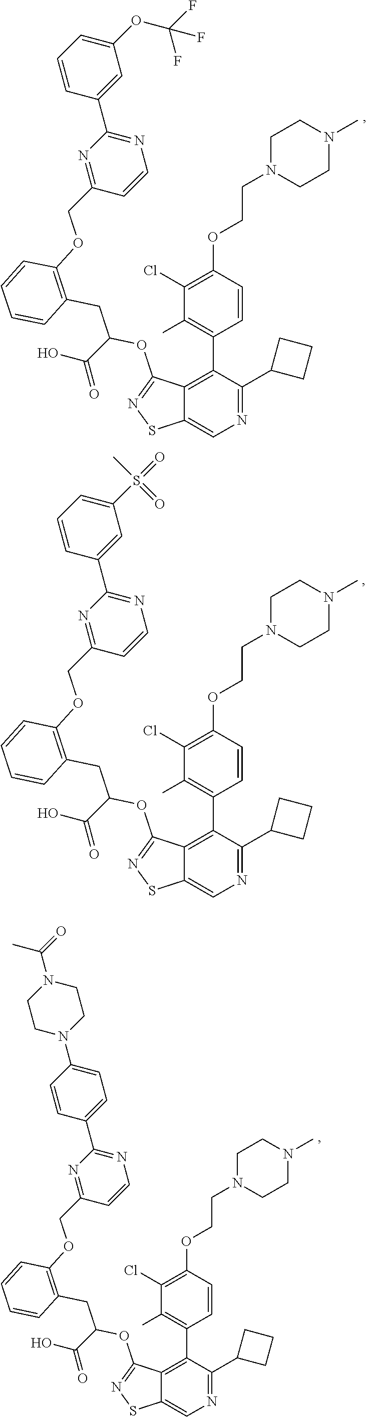

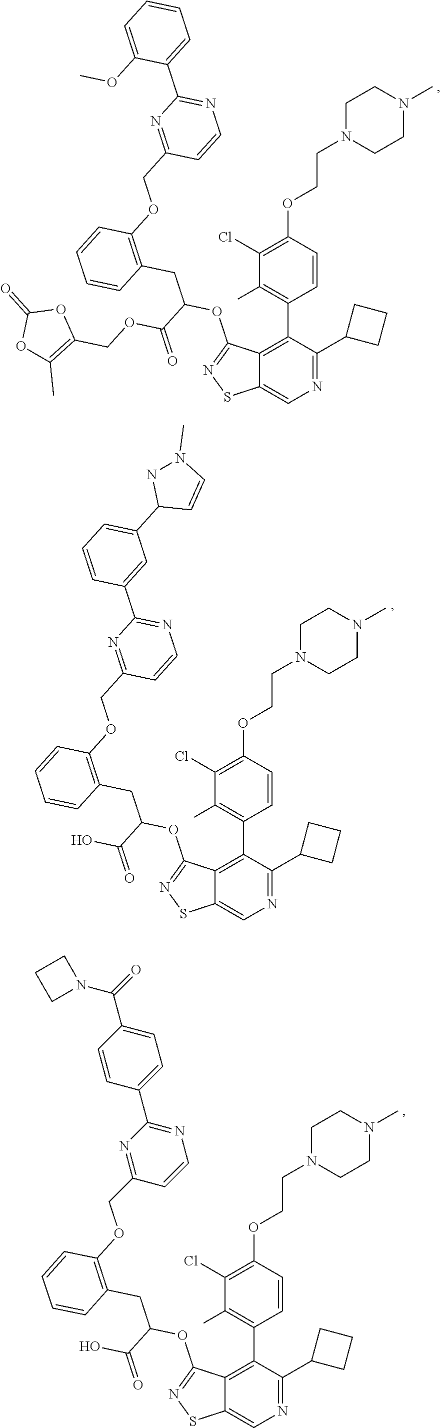

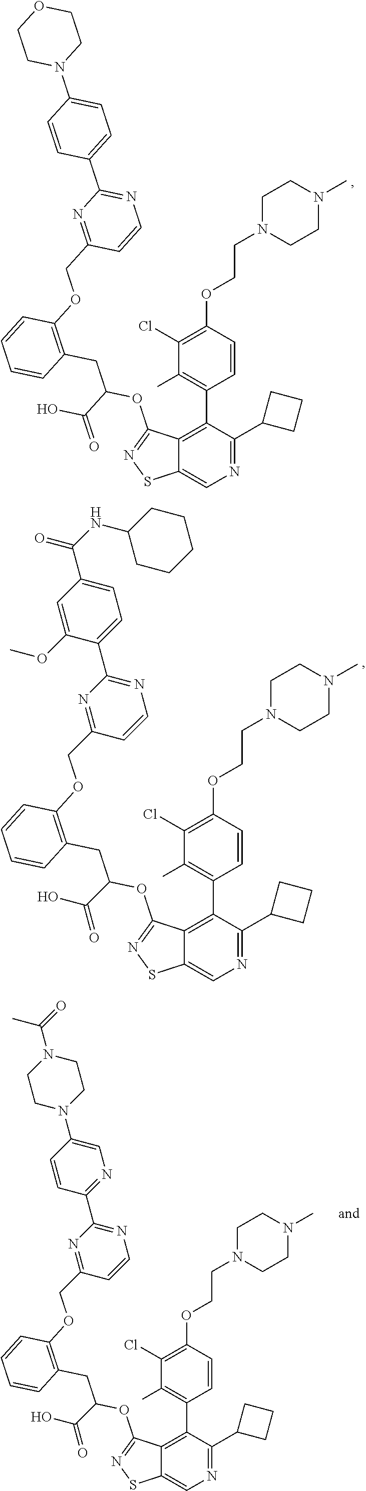

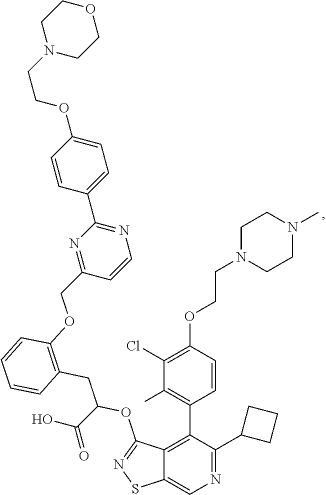 [SMILES: Cc1c(-c2c(C3CCC3)ncc3snc(OC(Cc4ccccc4OCc4ccnc(-c5ccc(OCCN6CCOCC6)cc5)n4)C(=O)O)c23)ccc(OCCN2CCN(C)CC2)c1Cl]

pharmaceutically acceptable salt thereof.

In some aspects, the invention relates to a compound of Formula II having a structure selected from:

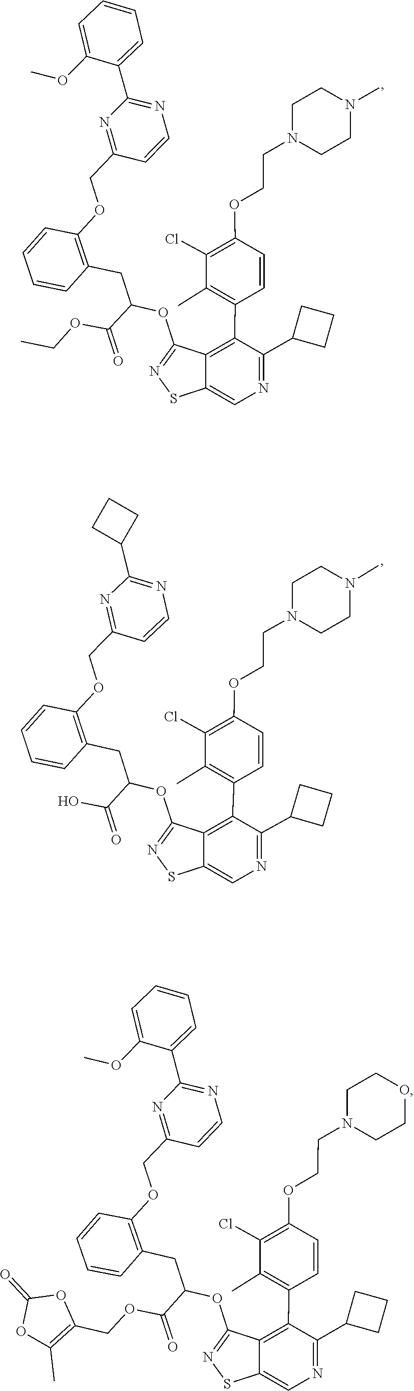 [SMILES: CCOC(=O)C(Cc1ccccc1OCc1ccnc(-c2ccccc2OC)n1)Oc1nsc2cnc(C3CCC3)c(-c3ccc(OCCN4CCN(C)CC4)c(Cl)c3C)c12 ; Cc1c(-c2c(C3CCC3)ncc3snc(OC(Cc4ccccc4OCc4ccnc(C5CCC5)n4)C(=O)O)c23)ccc(OCCN2CCN(C)CC2)c1Cl ; COc1ccccc1-c1nccc(COc2ccccc2CC(Oc2nsc3cnc(C4CCC4)c(-c4ccc(OCCN5CCOCC5)c(Cl)c4C)c23)C(=O)OCc2oc(=O)oc2C)n1]

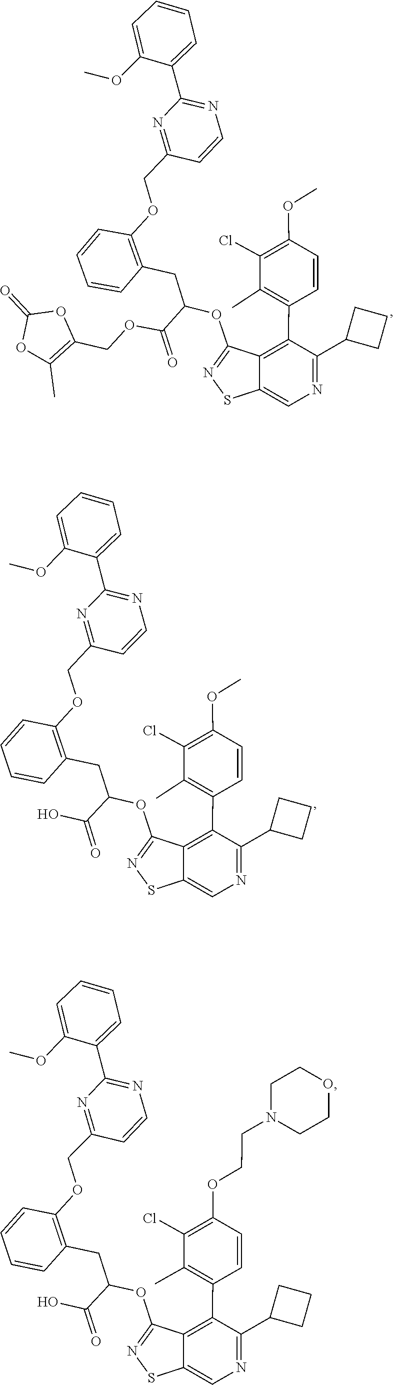 [SMILES: COc1ccccc1-c1nccc(COc2ccccc2CC(Oc2nsc3cnc(C4CCC4)c(-c4ccc(OC)c(Cl)c4C)c23)C(=O)OCc2oc(=O)oc2C)n1 ; COc1ccccc1-c1nccc(COc2ccccc2CC(Oc2nsc3cnc(C4CCC4)c(-c4ccc(OC)c(Cl)c4C)c23)C(=O)O)n1 ; COc1ccccc1-c1nccc(COc2ccccc2CC(Oc2nsc3cnc(C4CCC4)c(-c4ccc(OCCN5CCOCC5)c(Cl)c4C)c23)C(=O)O)n1]

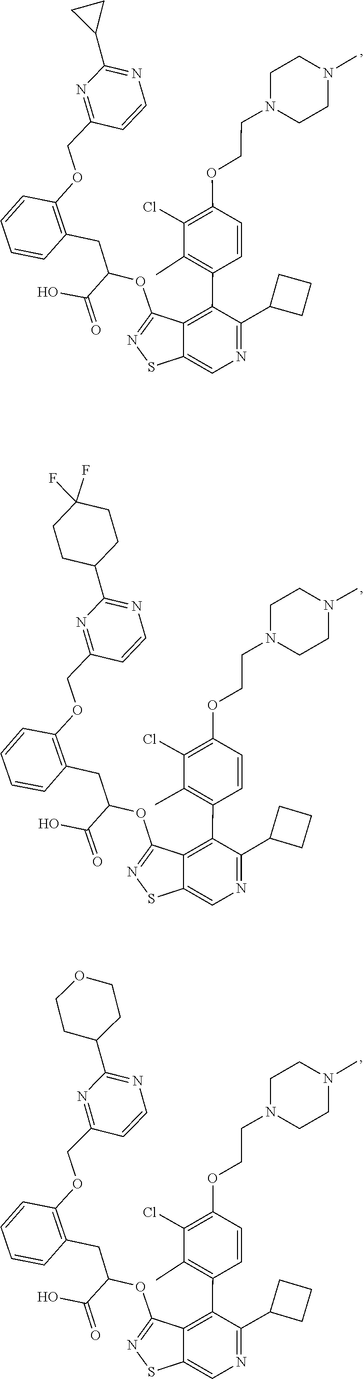 [SMILES: Cc1c(-c2c(C3CCC3)ncc3snc(OC(Cc4ccccc4OCc4ccnc(C5CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCC(F)(F)CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCOCC5)n4)C(=O)O)c23)ccc(OCCN2CCN(C)CC2)c1Cl]

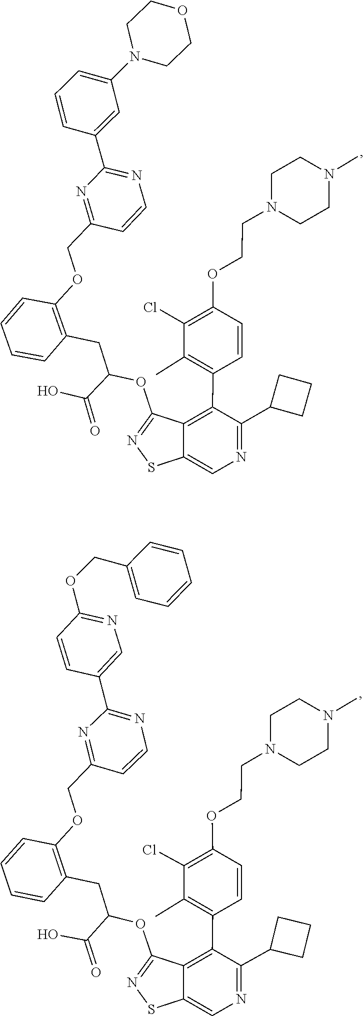 [SMILES: Cc1c(-c2c(C3CCC3)ncc3snc(OC(Cc4ccccc4OCc4ccnc(-c5cccc(N6CCOCC6)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(OCc6ccccc6)nc5)n4)C(=O)O)c23)ccc(OCCN2CCN(C)CC2)c1Cl]

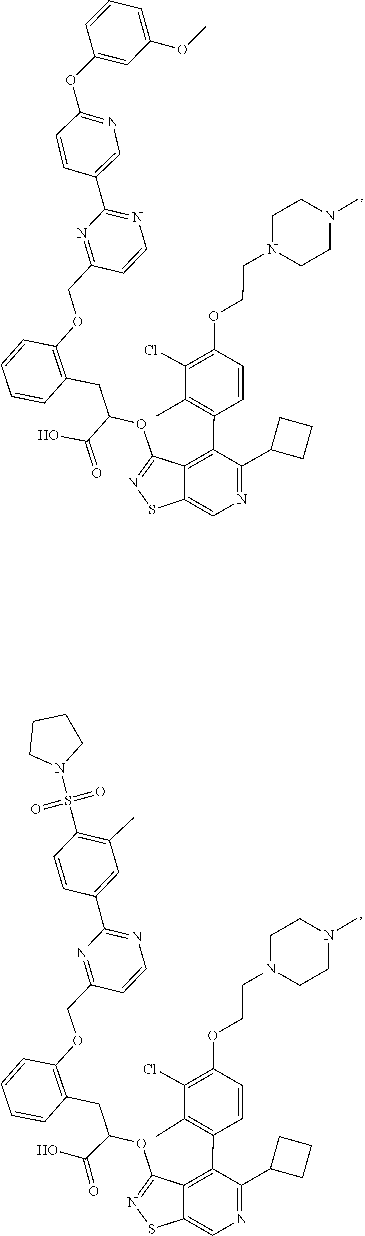 [SMILES: COc1cccc(Oc2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cn2)c1 ; Cc1cc(-c2nccc(COc3ccccc3CC(Oc3nsc4cnc(C5CCC5)c(-c5ccc(OCCN6CCN(C)CC6)c(Cl)c5C)c34)C(=O)O)n2)ccc1S(=O)(=O)N1CCCC1]

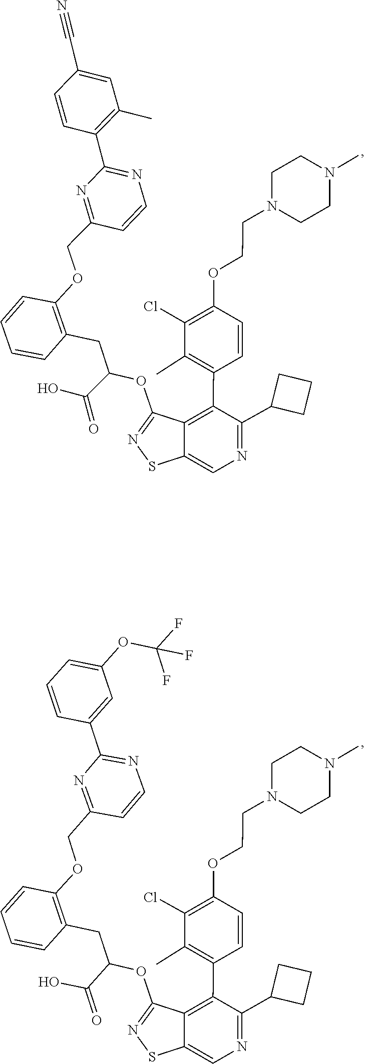 [SMILES: Cc1cc(C#N)ccc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5cccc(OC(F)(F)F)c5)n4)C(=O)O)c23)ccc(OCCN2CCN(C)CC2)c1Cl]

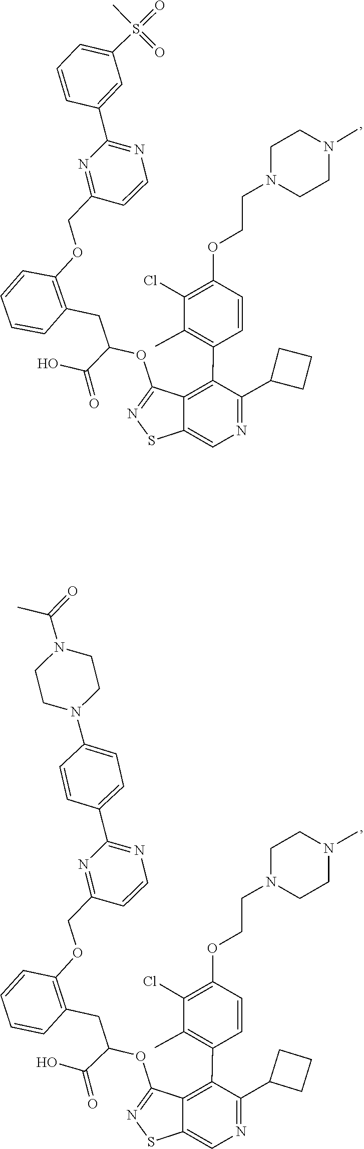 [SMILES: Cc1c(-c2c(C3CCC3)ncc3snc(OC(Cc4ccccc4OCc4ccnc(-c5cccc(S(C)(=O)=O)c5)n4)C(=O)O)c23)ccc(OCCN2CCN(C)CC2)c1Cl ; CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cc2)CC1]

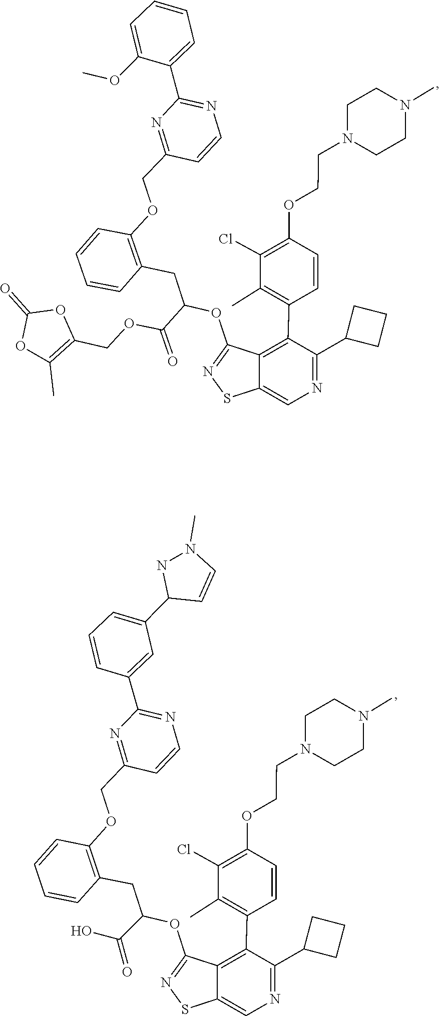

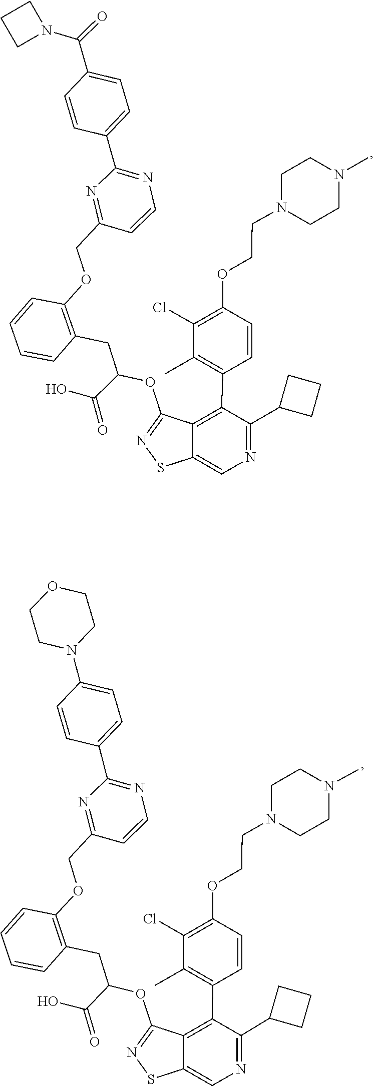 [SMILES: Cc1c(-c2c(C3CCC3)ncc3snc(OC(Cc4ccccc4OCc4ccnc(-c5ccc(C(=O)N6CCC6)cc5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(N6CCOCC6)cc5)n4)C(=O)O)c23)ccc(OCCN2CCN(C)CC2)c1Cl]

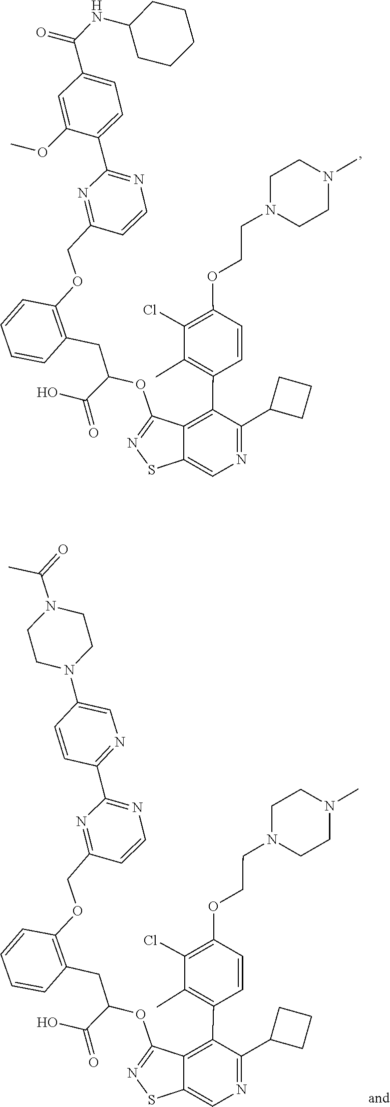 [SMILES: COc1cc(C(=O)NC2CCCCC2)ccc1-c1nccc(COc2ccccc2CC(Oc2nsc3cnc(C4CCC4)c(-c4ccc(OCCN5CCN(C)CC5)c(Cl)c4C)c23)C(=O)O)n1 ; CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)nc2)CC1]

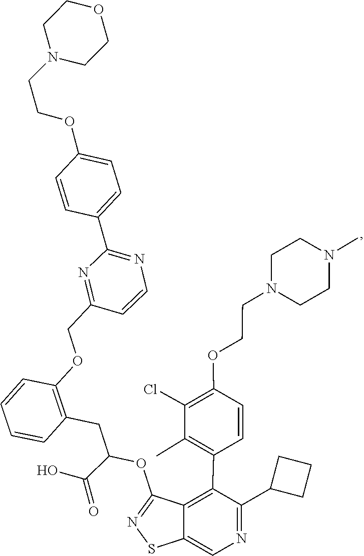 [SMILES: Cc1c(-c2c(C3CCC3)ncc3snc(OC(Cc4ccccc4OCc4ccnc(-c5ccc(OCCN6CCOCC6)cc5)n4)C(=O)O)c23)ccc(OCCN2CCN(C)CC2)c1Cl]

pharmaceutically acceptable salt thereof.

In some aspects, the invention relates to a compound of Formula III having a structure selected from:

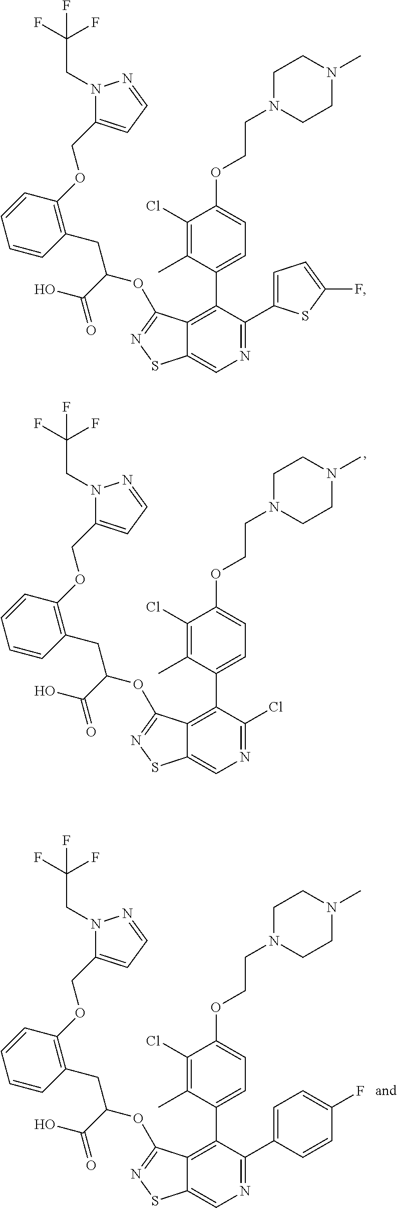 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl]

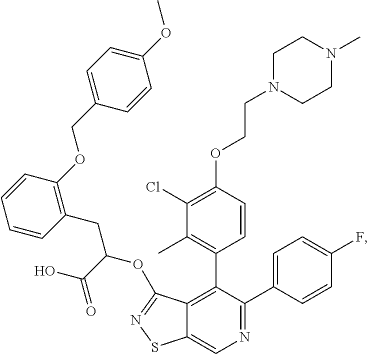 [SMILES: COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1]

or a pharmaceutically acceptable salt thereof.

In some aspects, the invention relates to a compound of Formula II having a structure selected from:

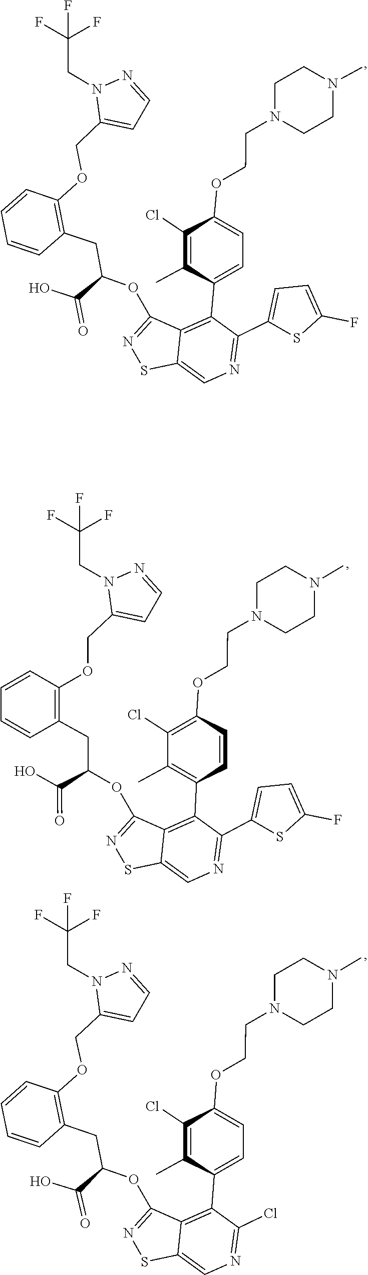

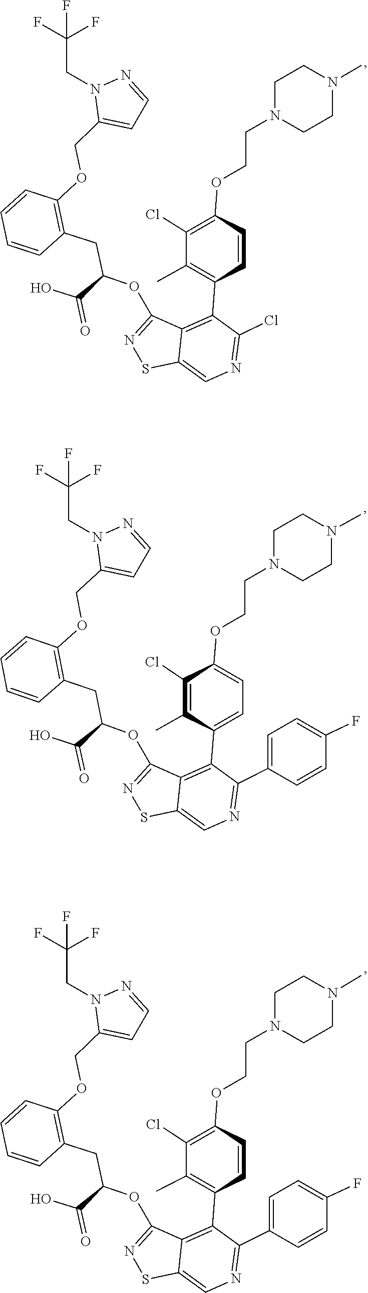

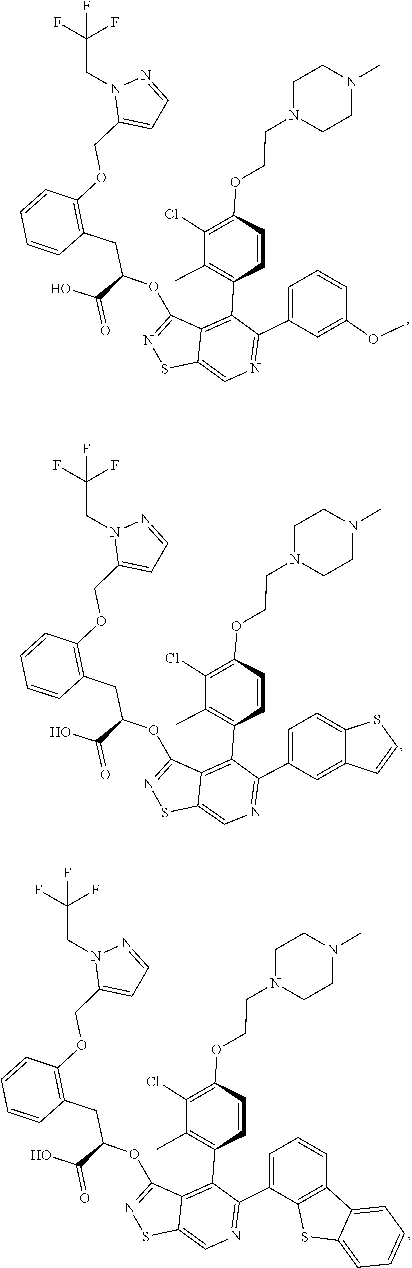

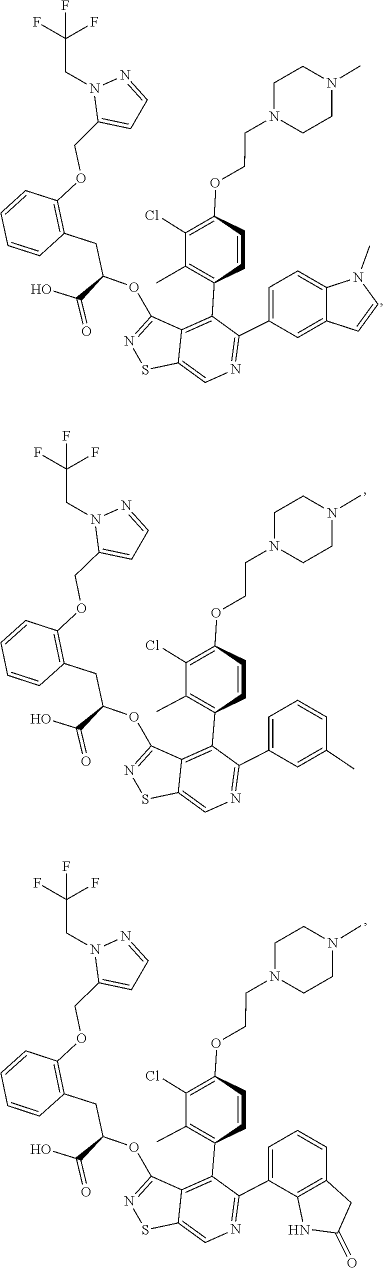

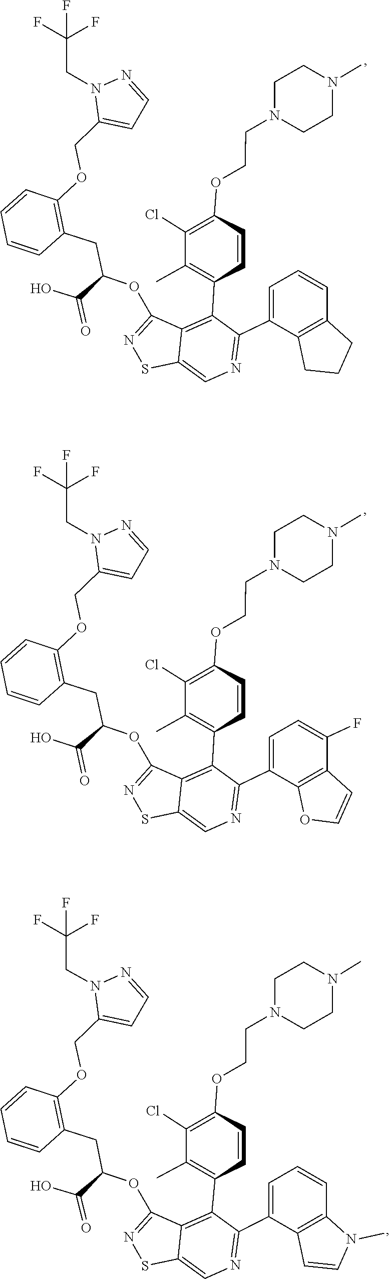

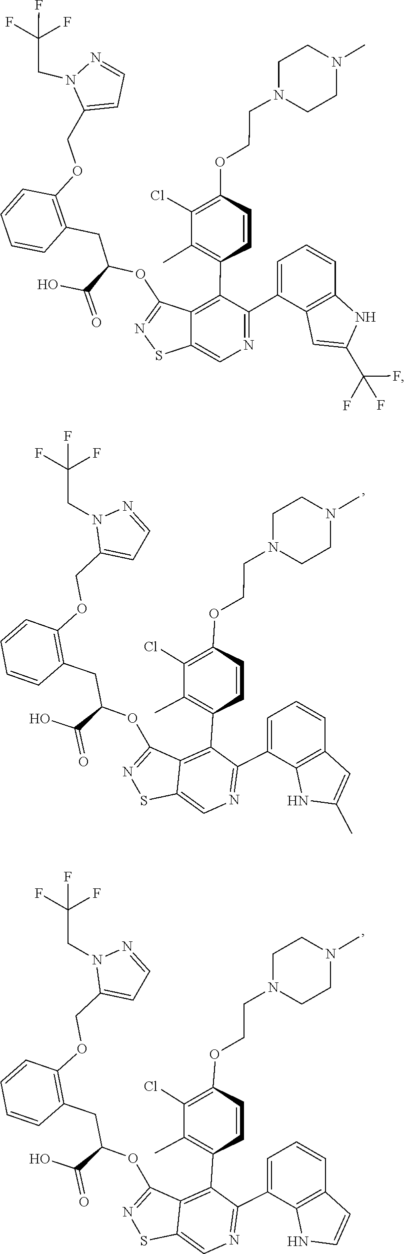

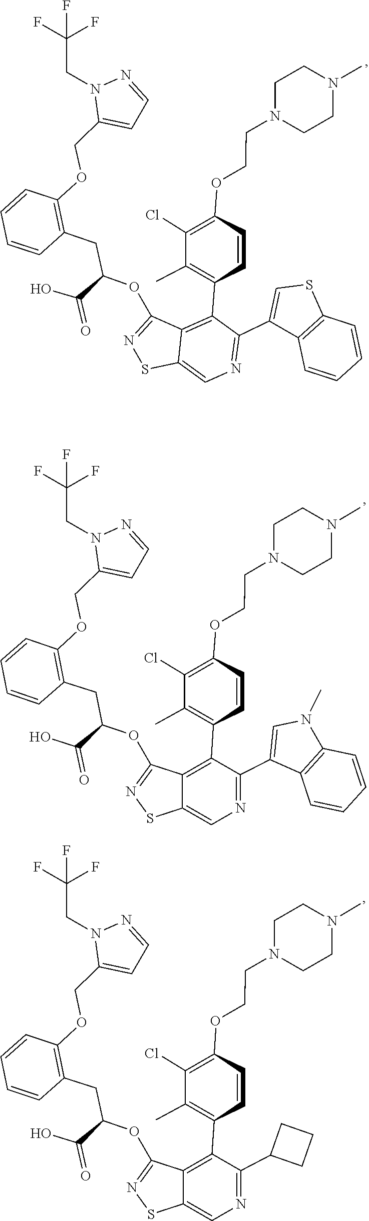

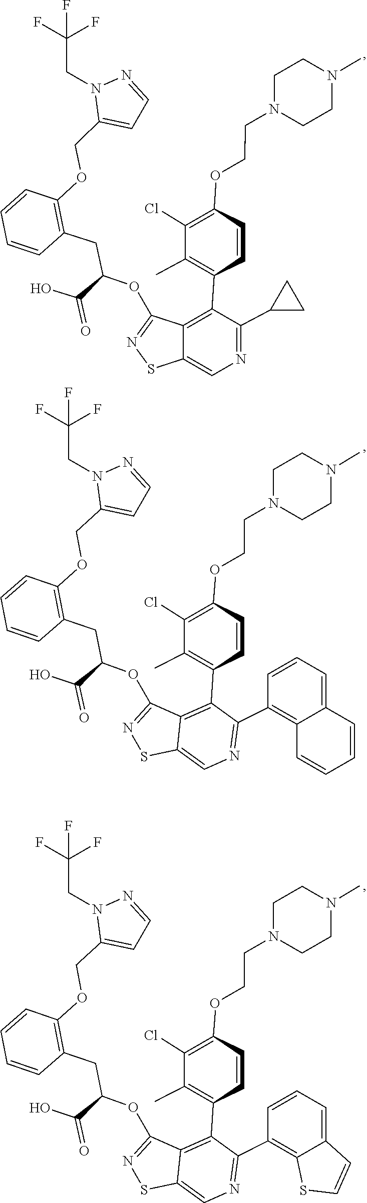

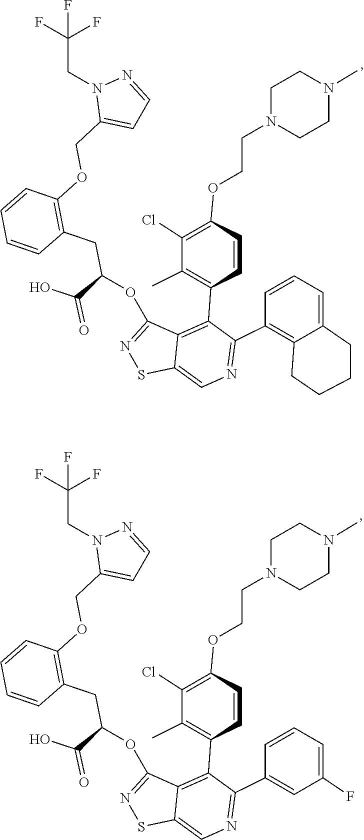

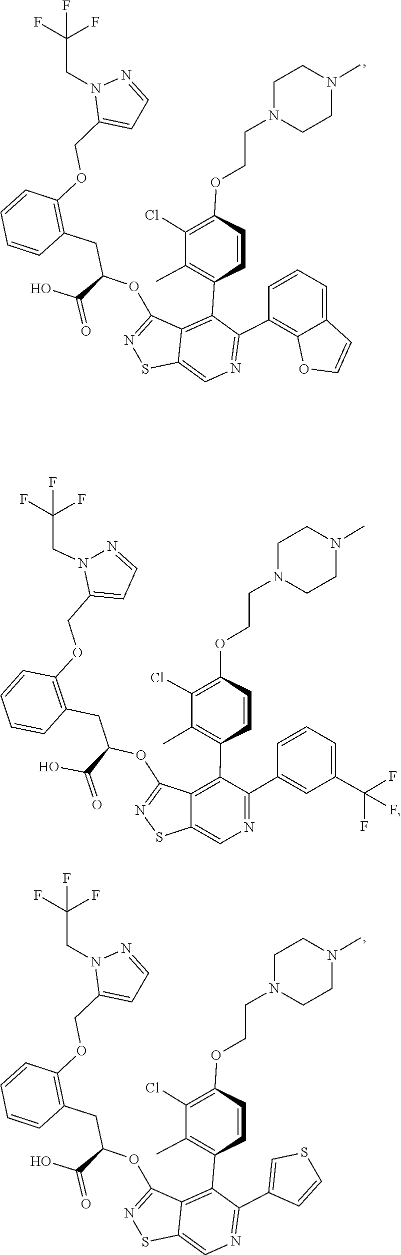

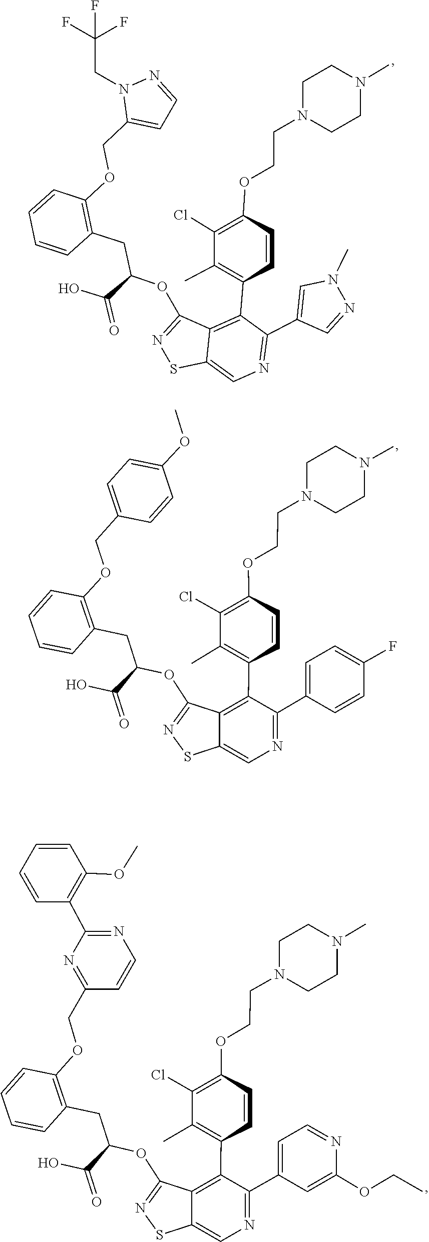

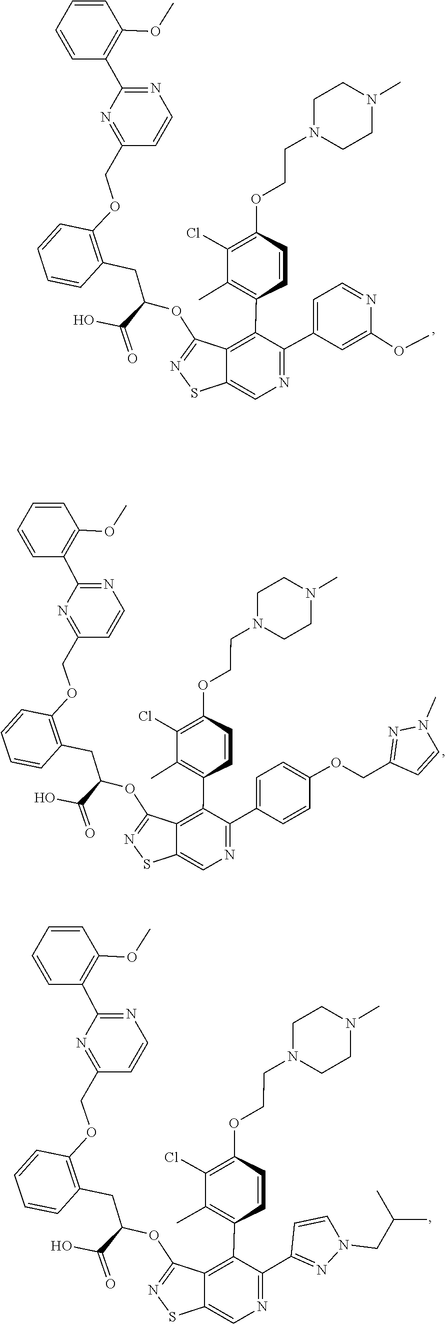

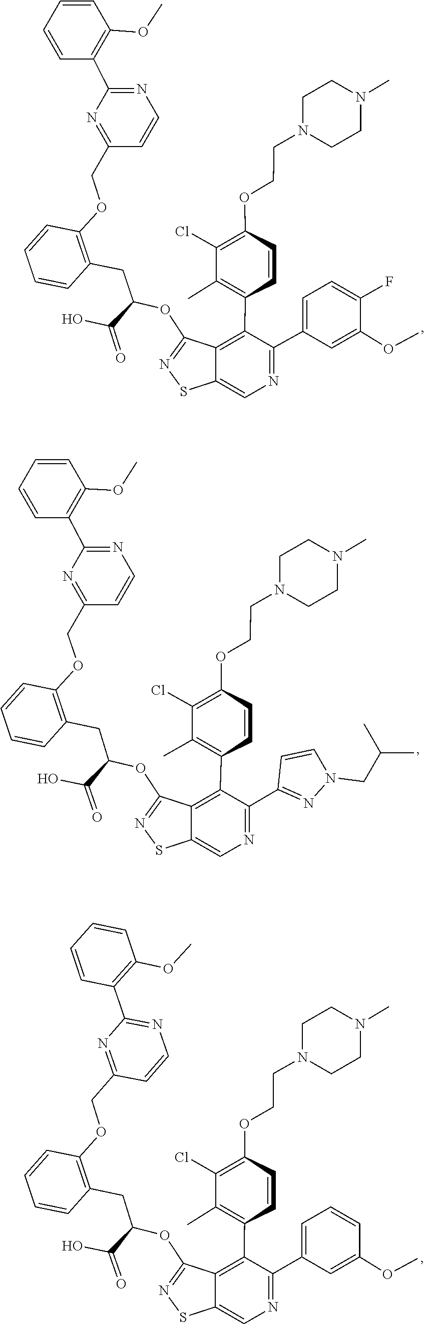

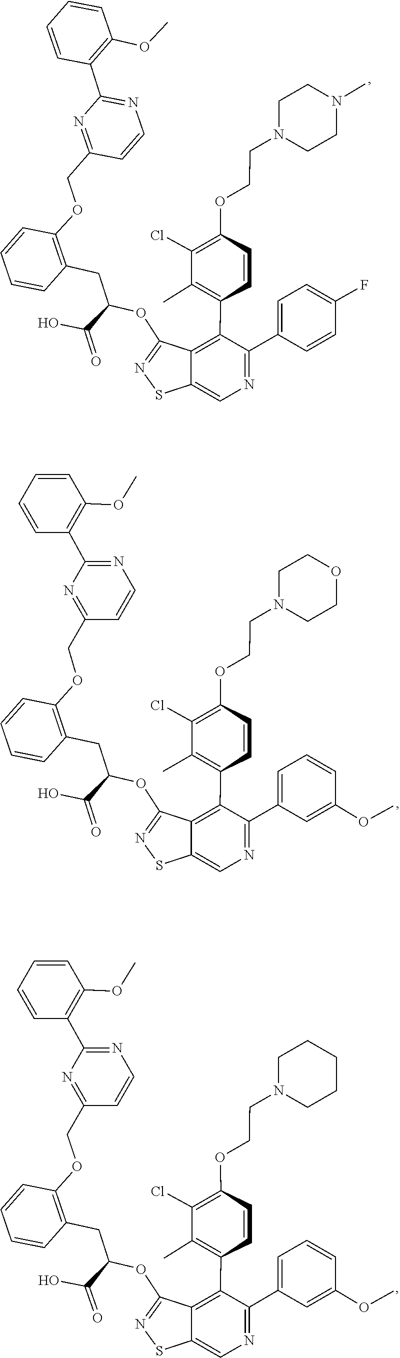

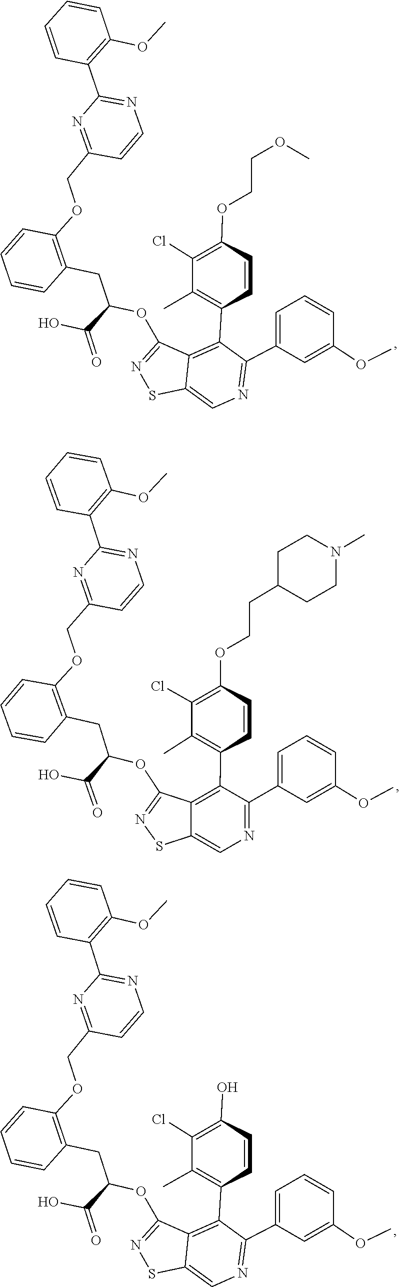

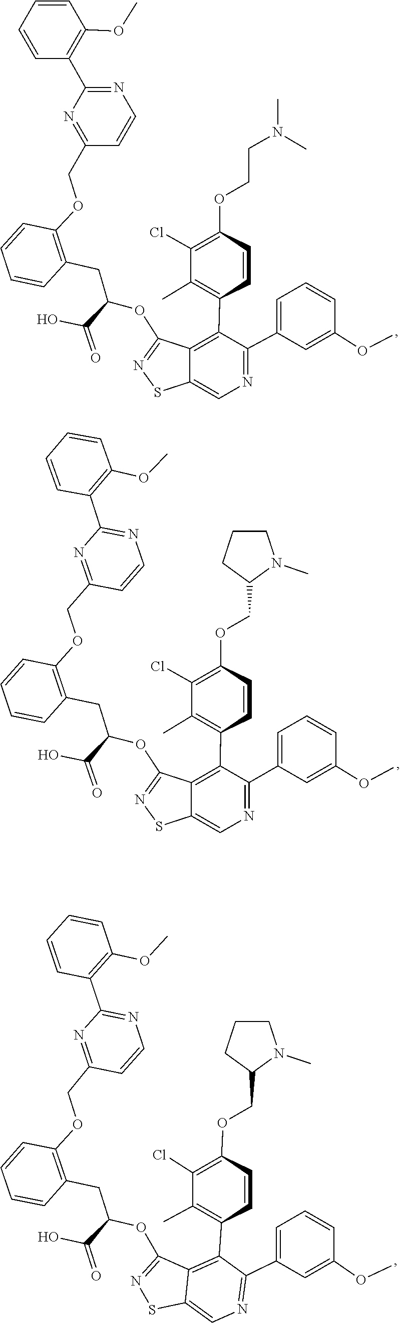

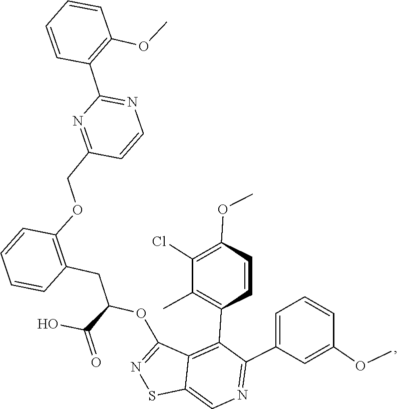

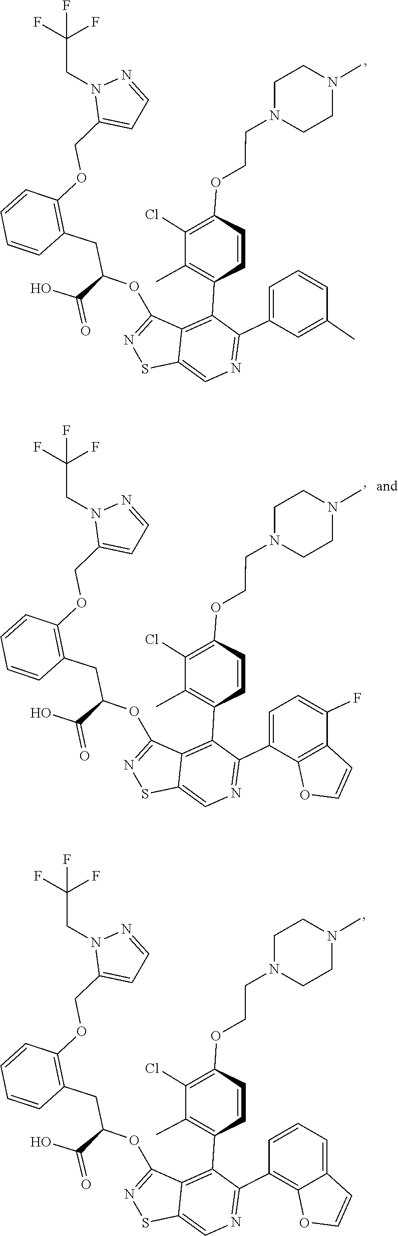

or a pharmaceutically acceptable salt thereof.

In some aspects, the invention relates to a compound of Formula II having a structure selected from:

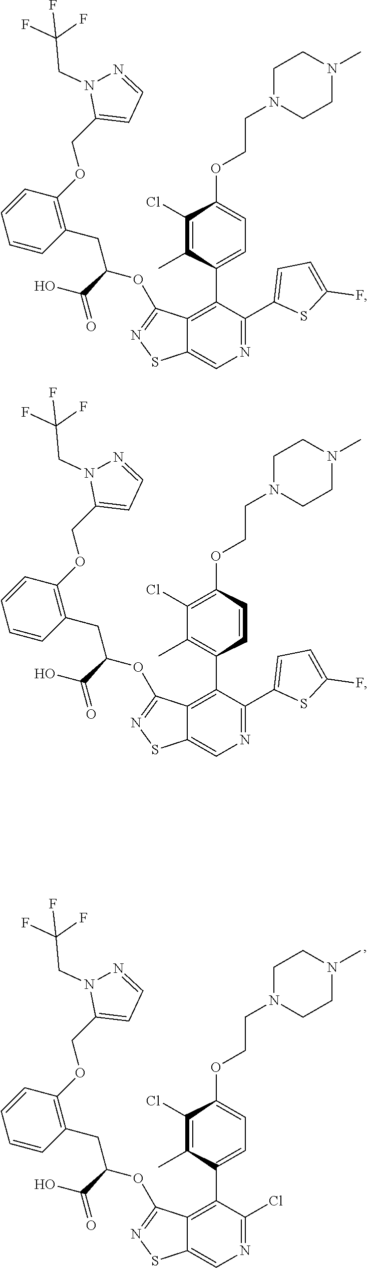

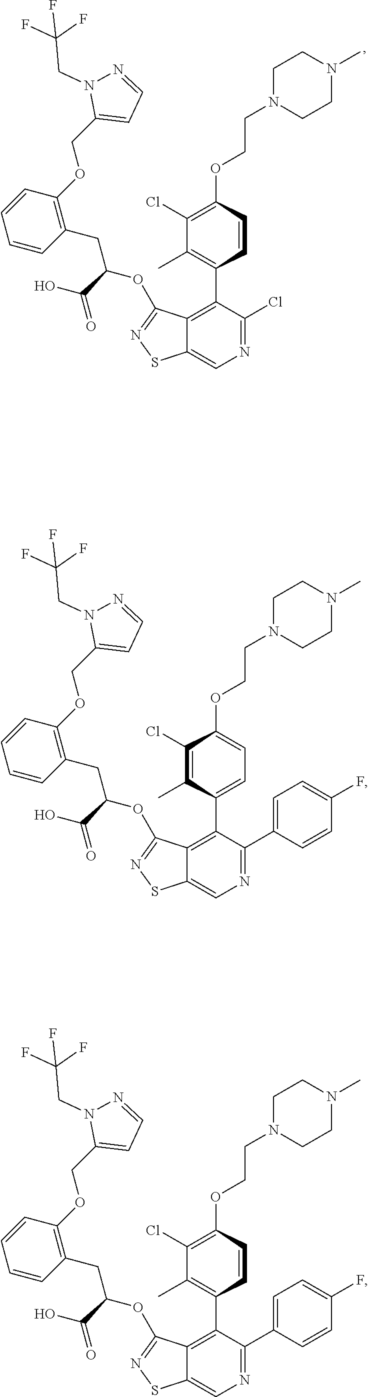

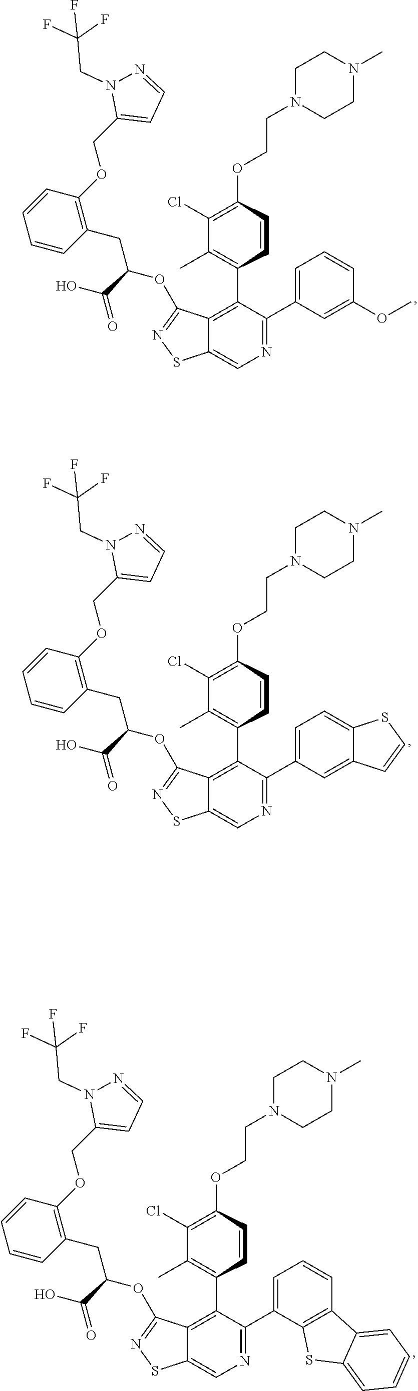

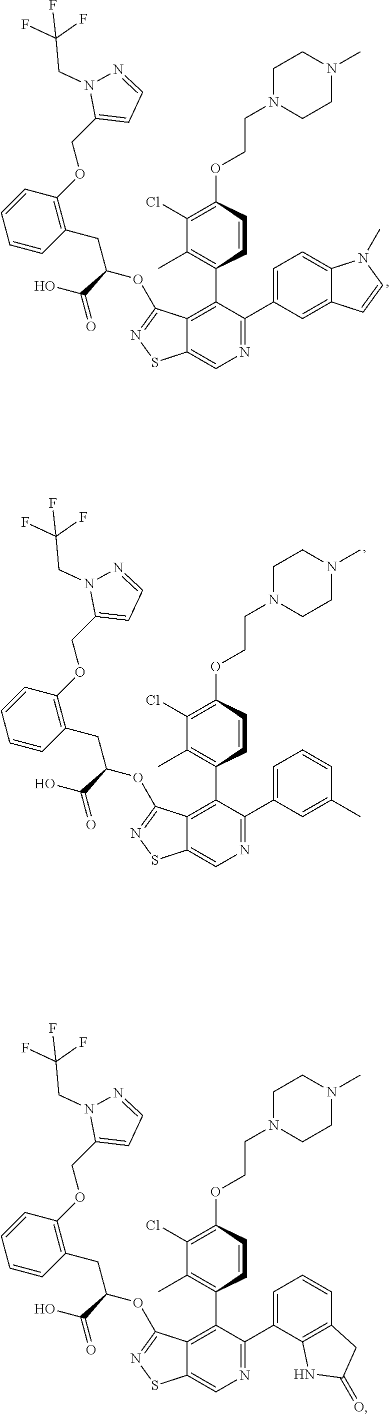

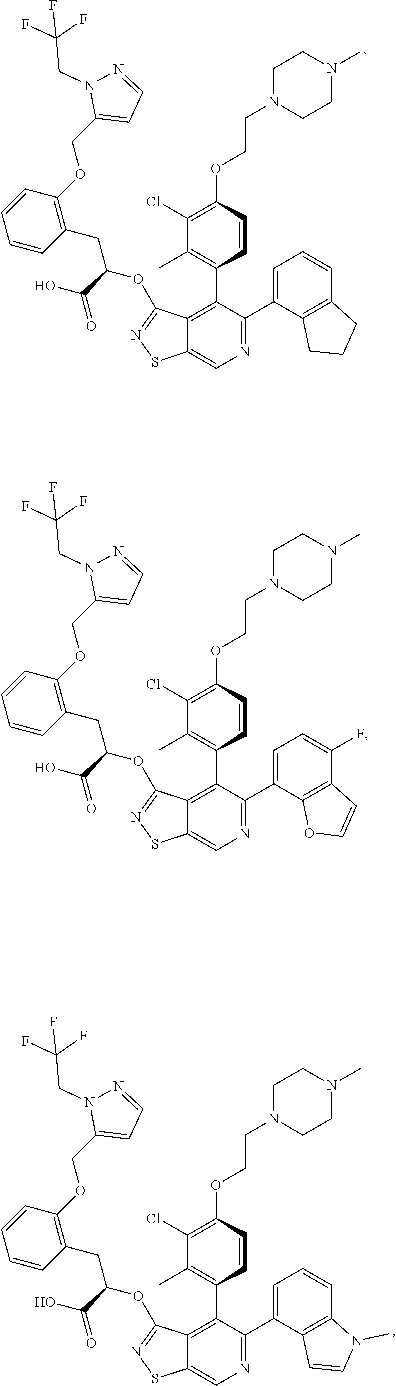

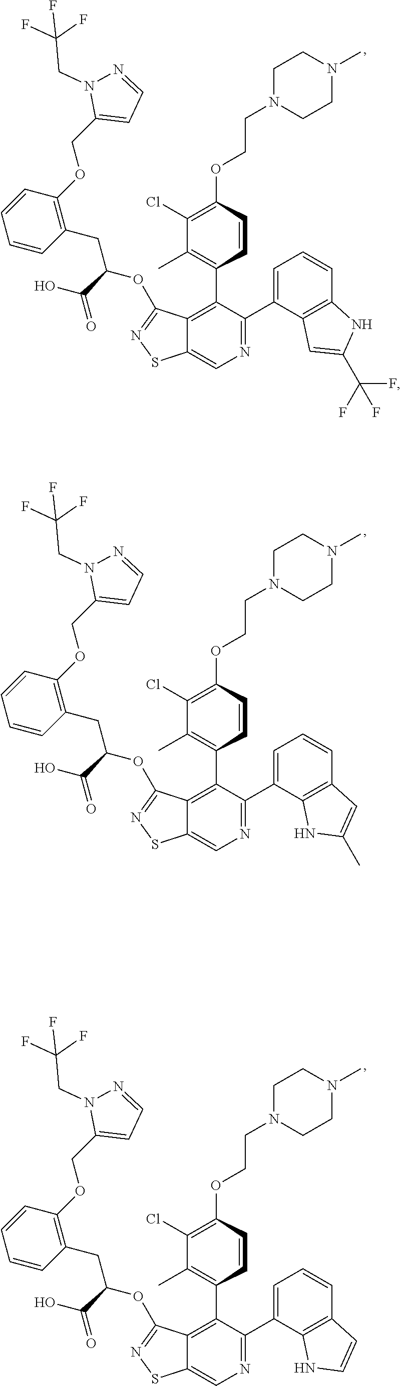

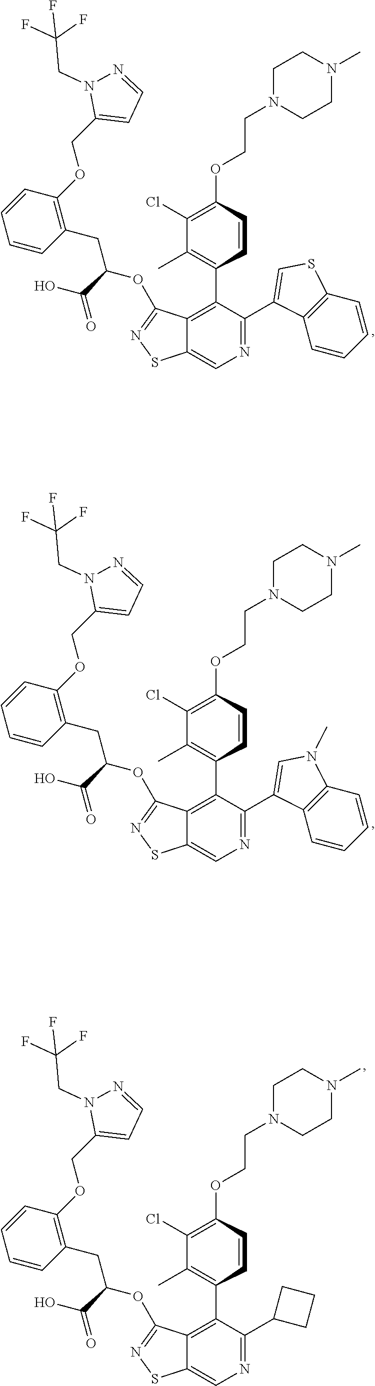

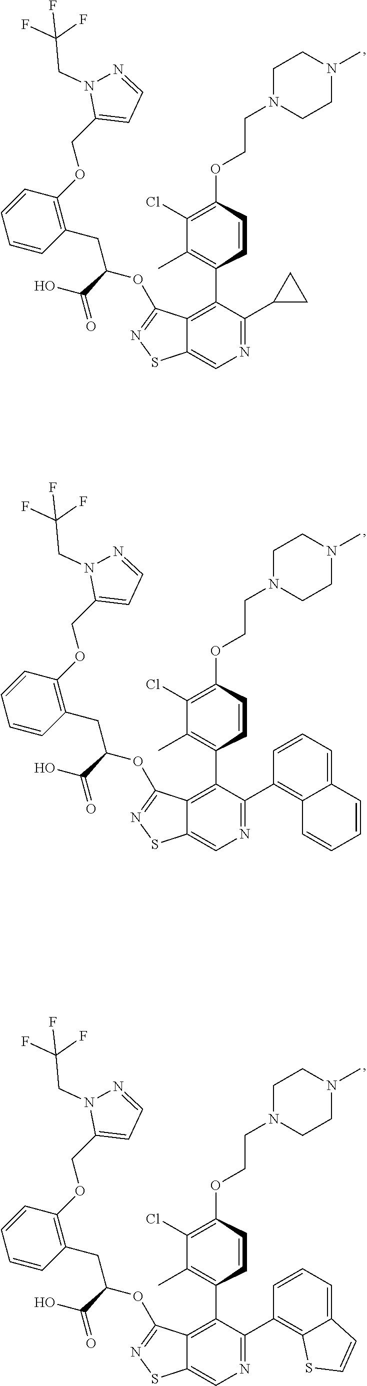

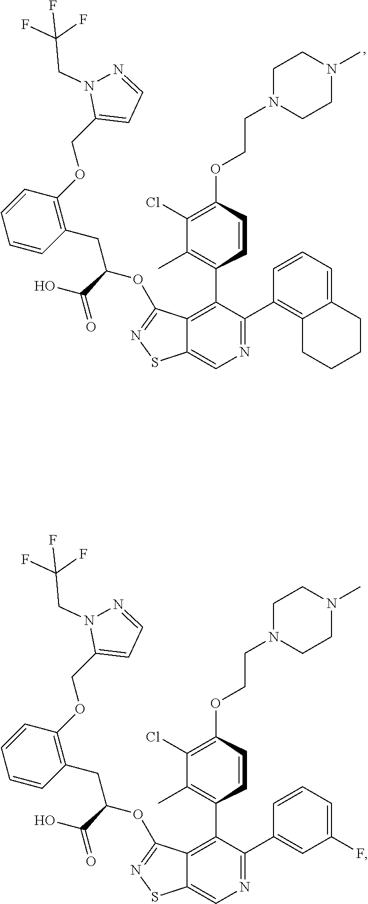

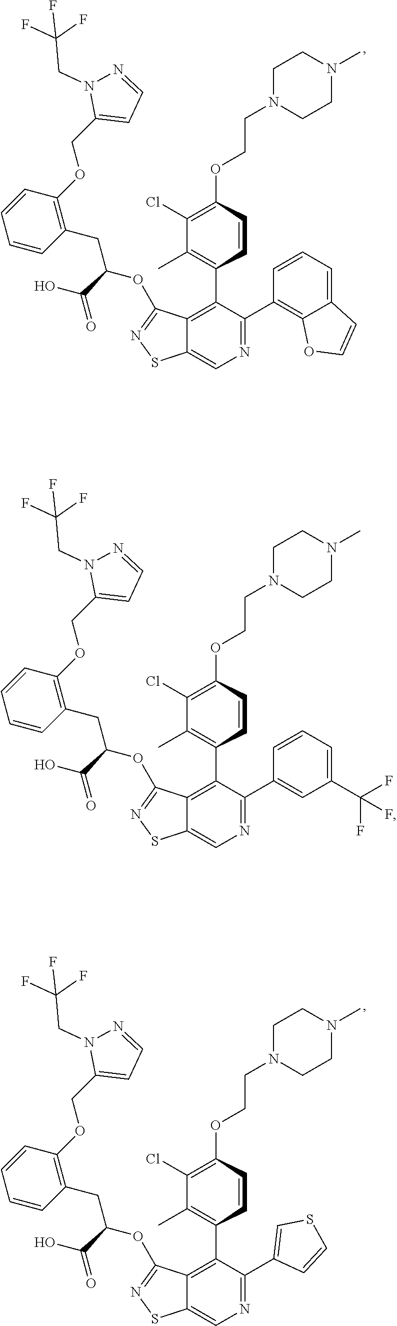

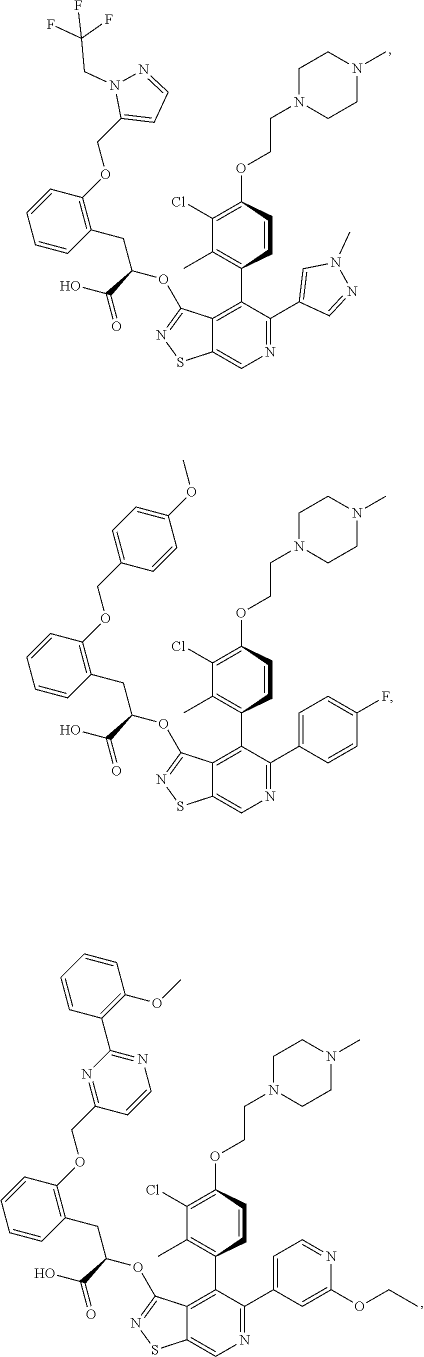

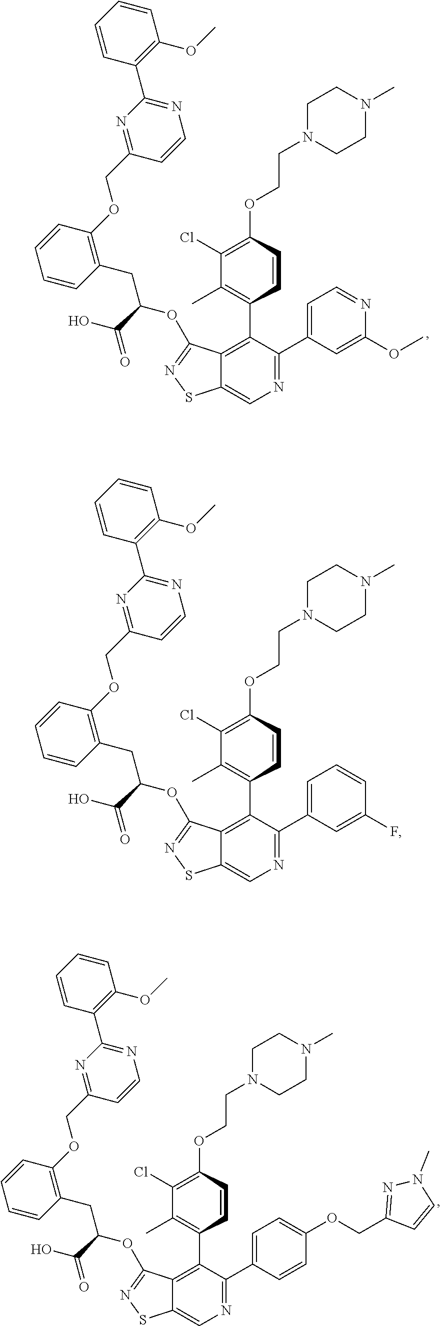

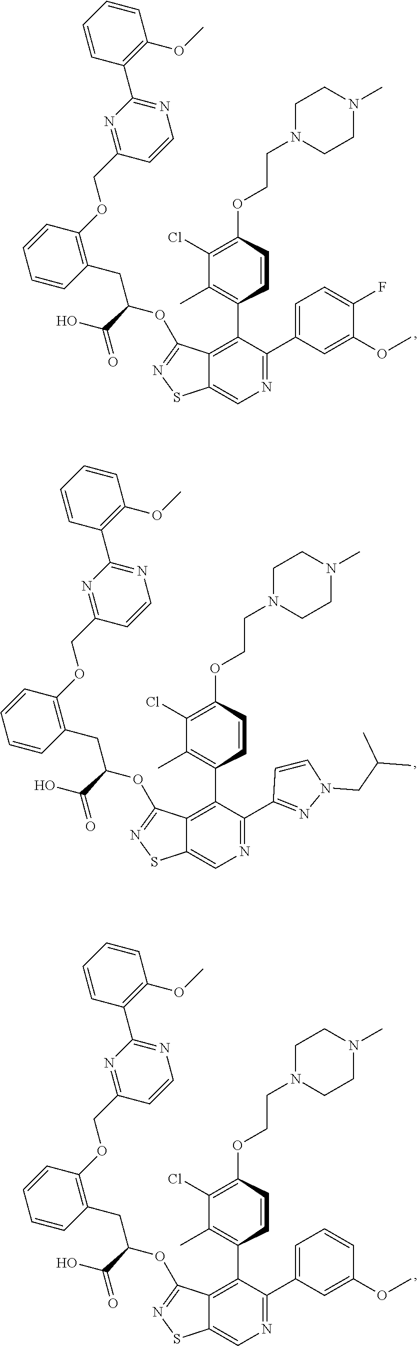

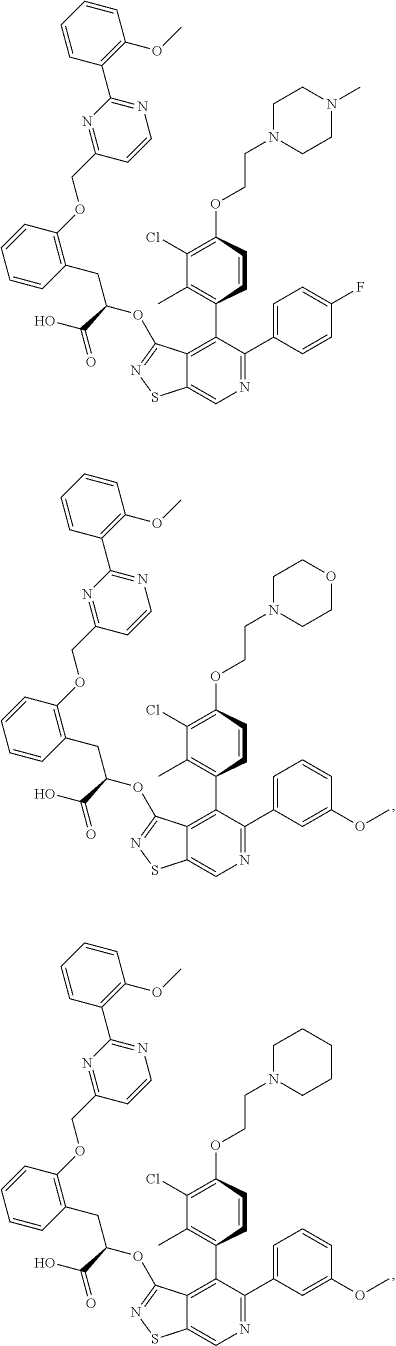

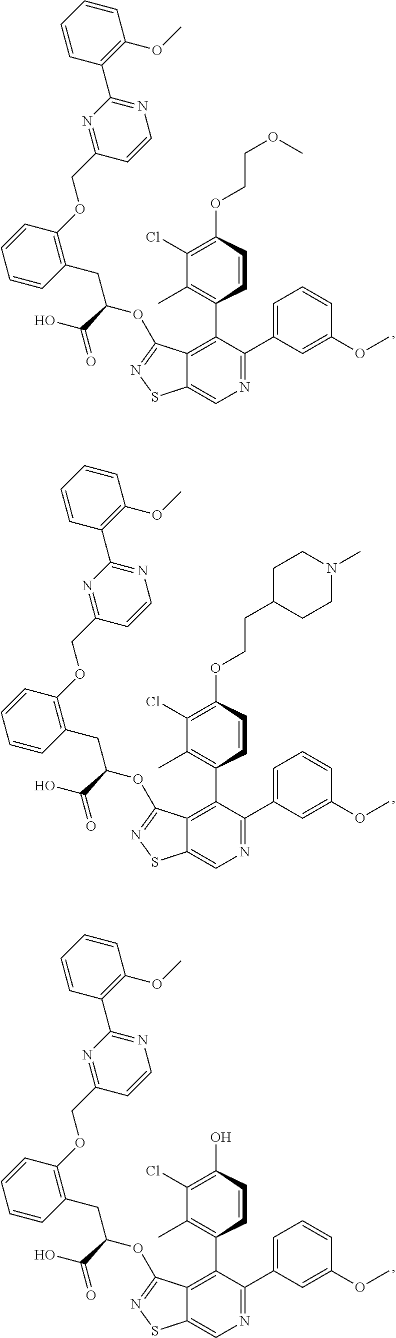

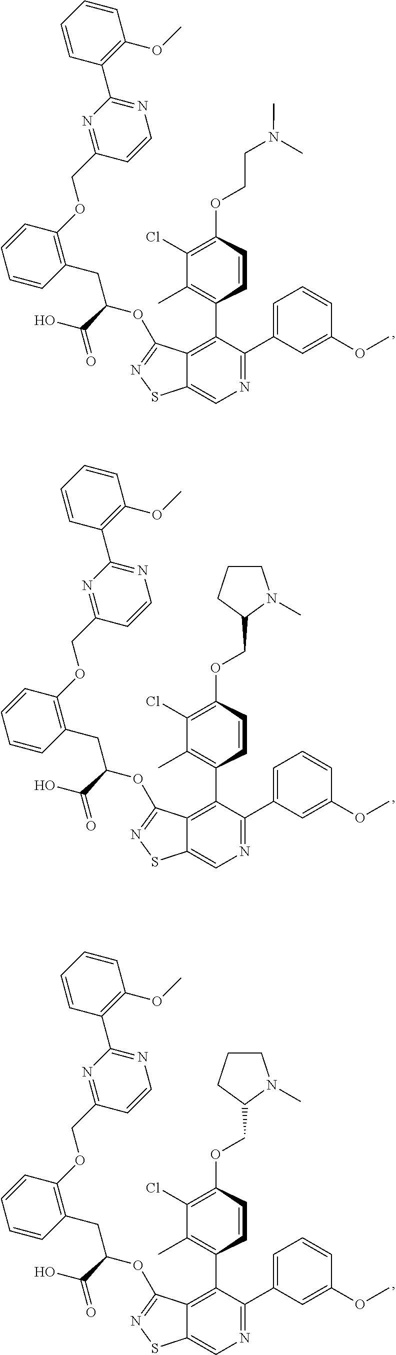

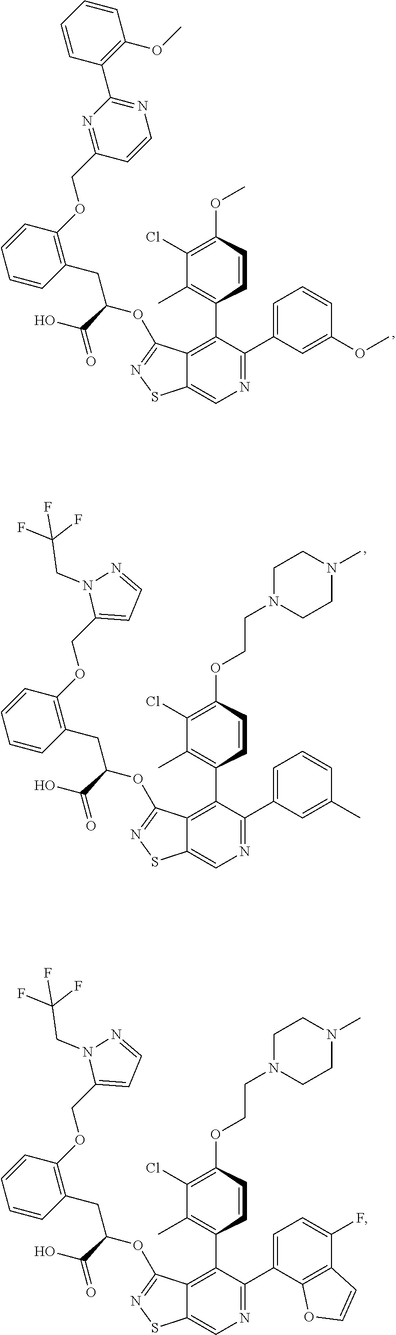

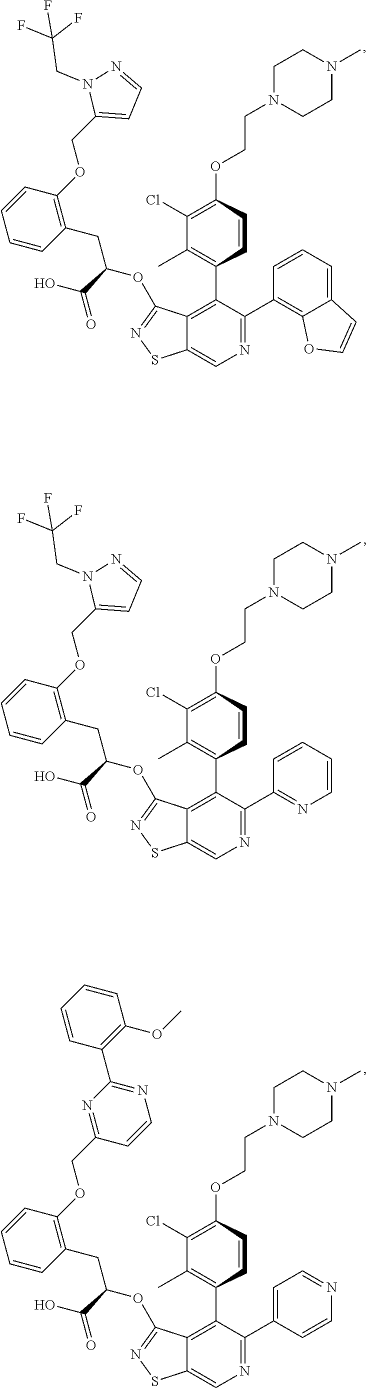

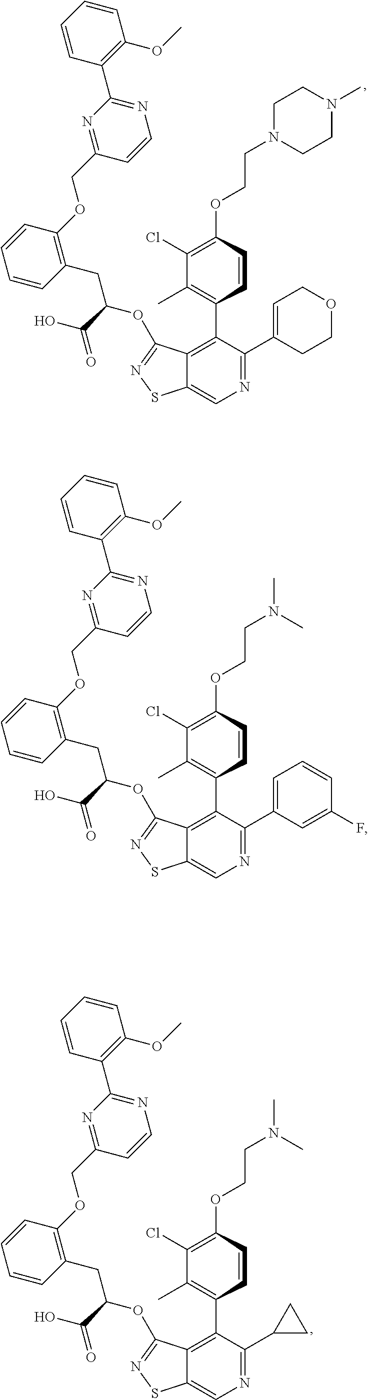

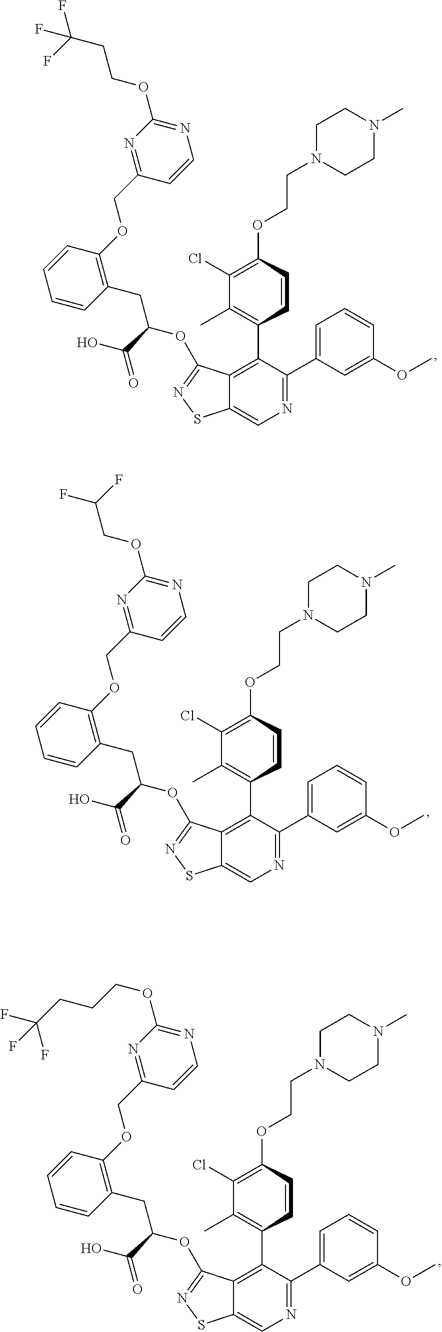

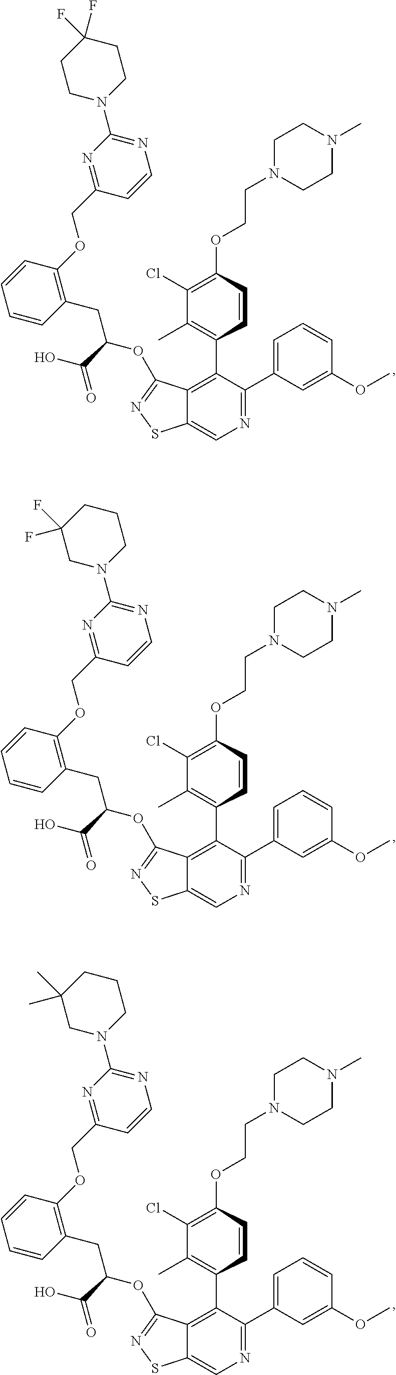

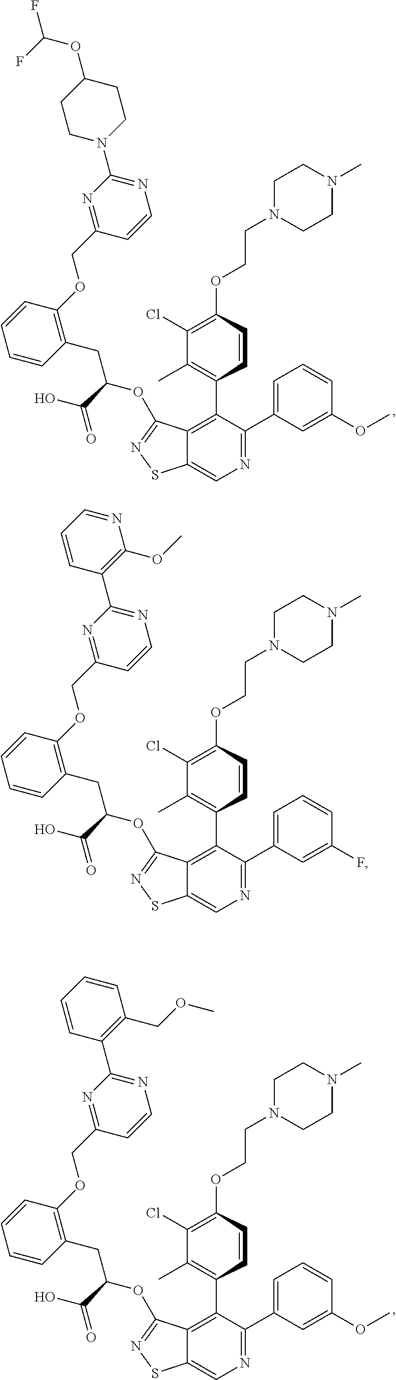

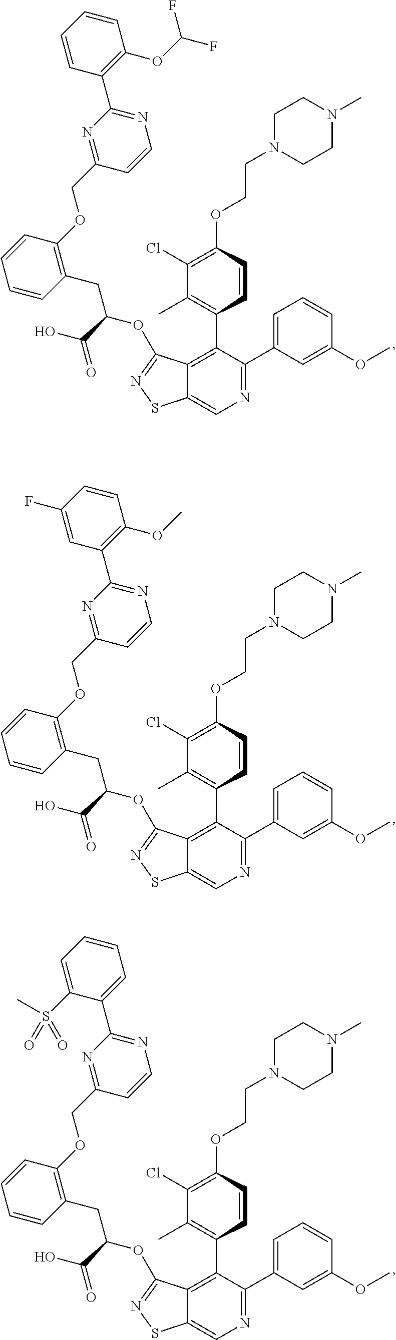

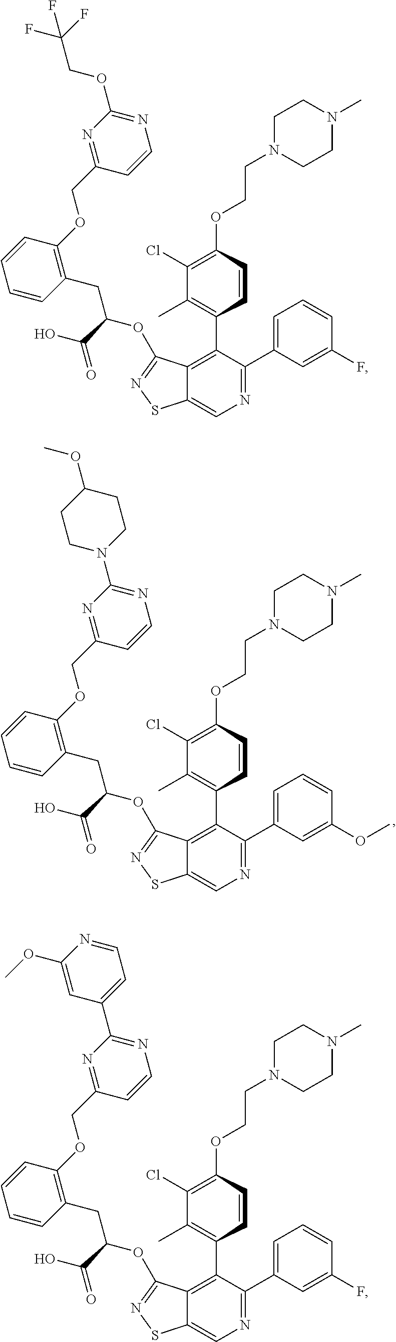

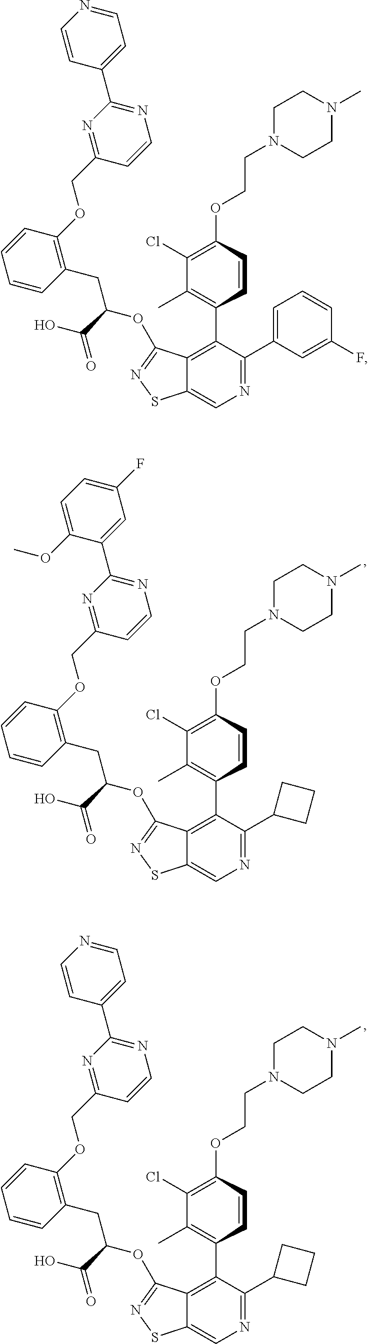

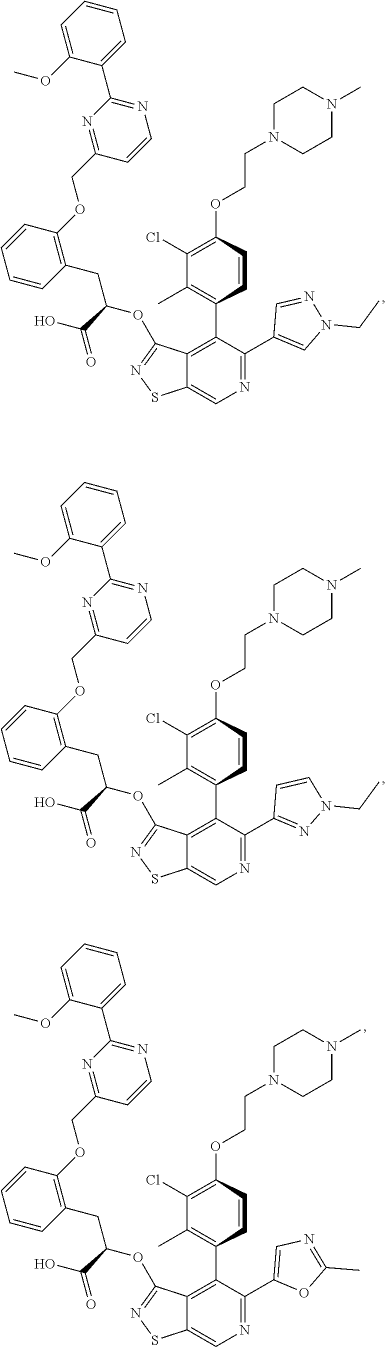

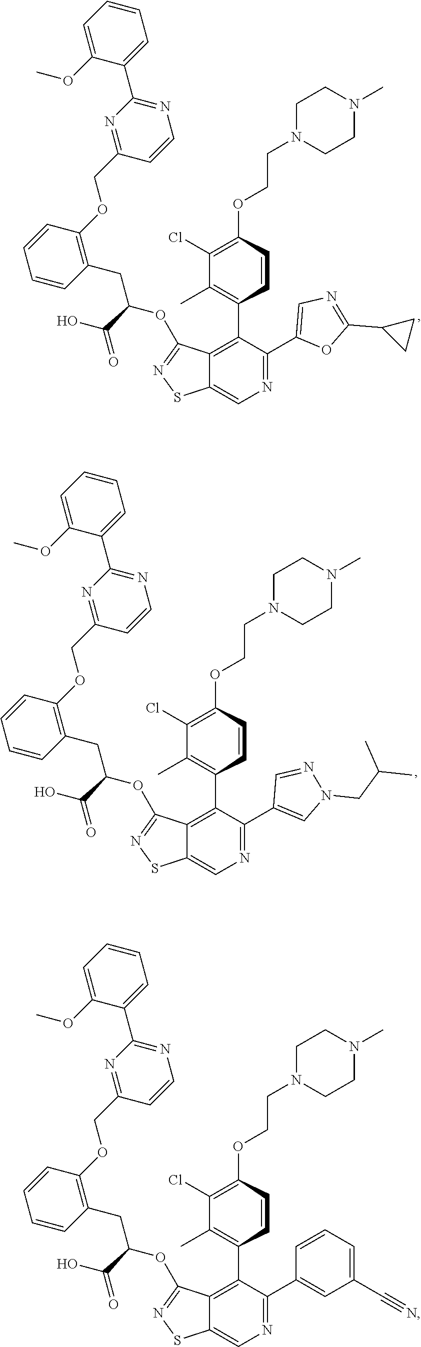

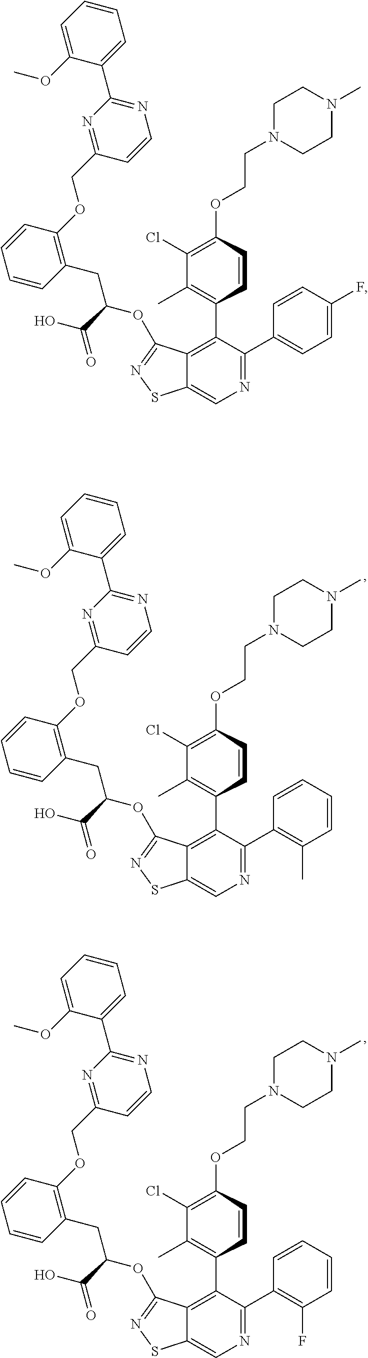

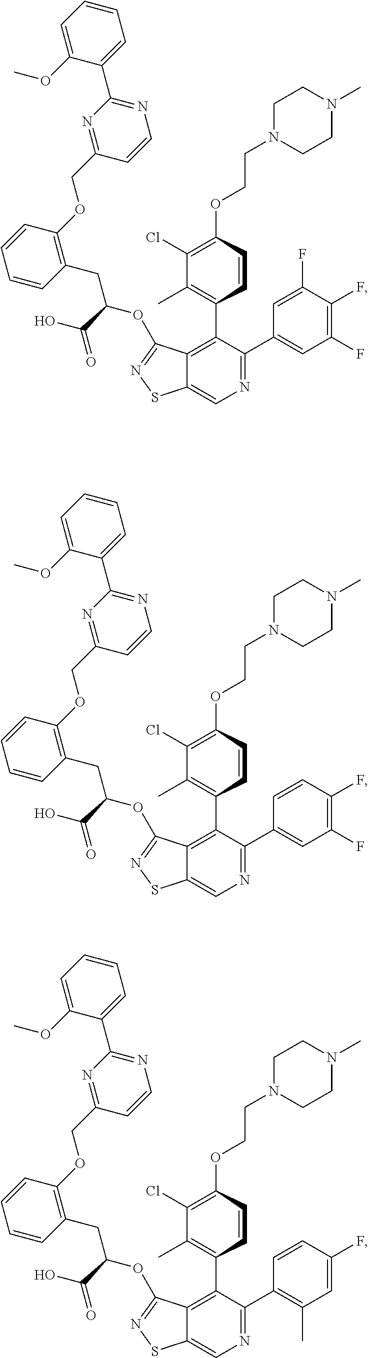

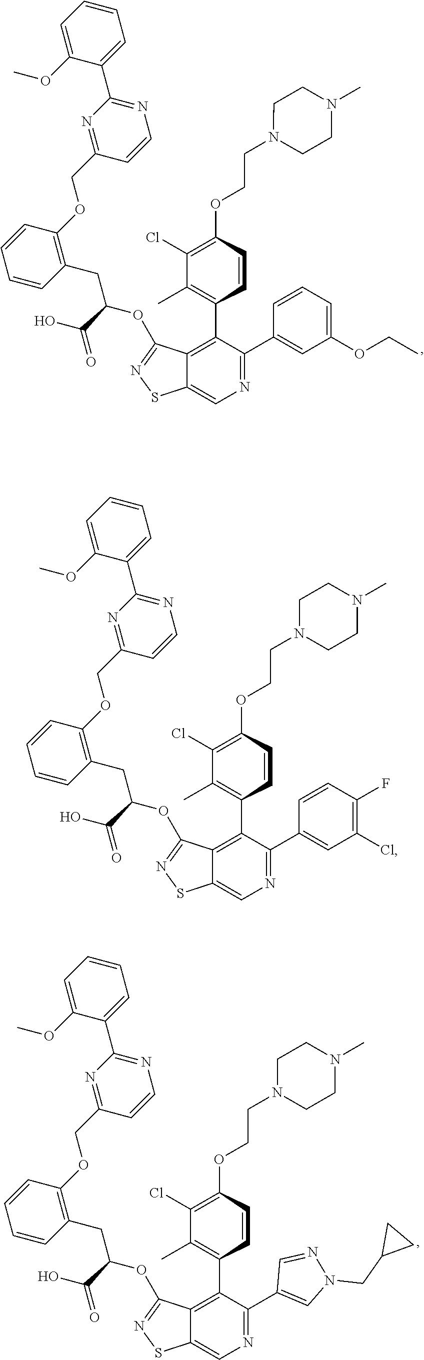

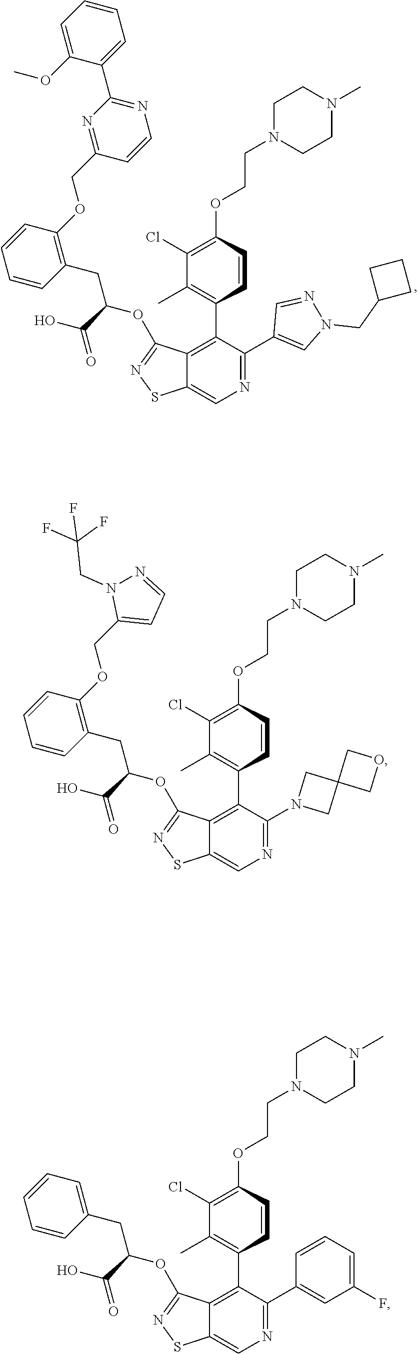

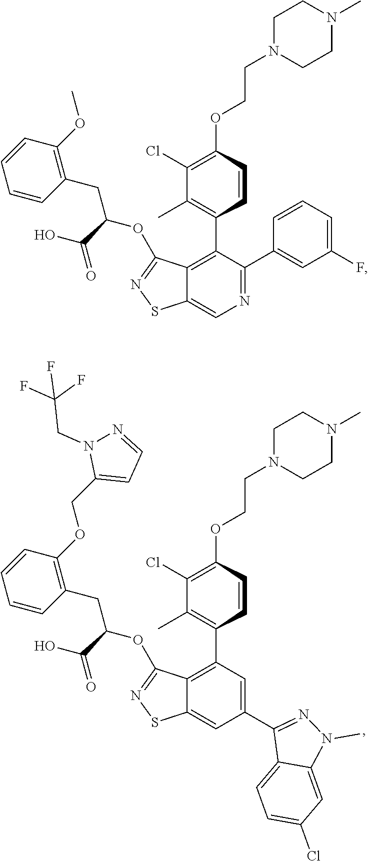

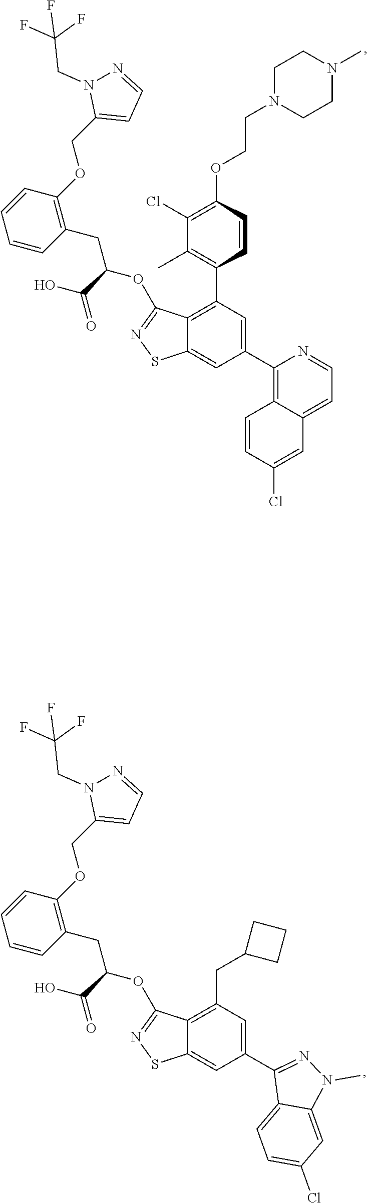

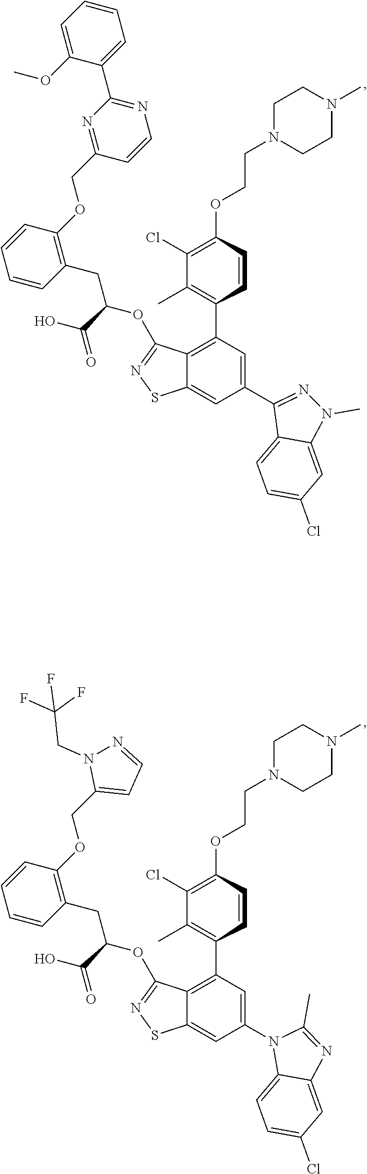

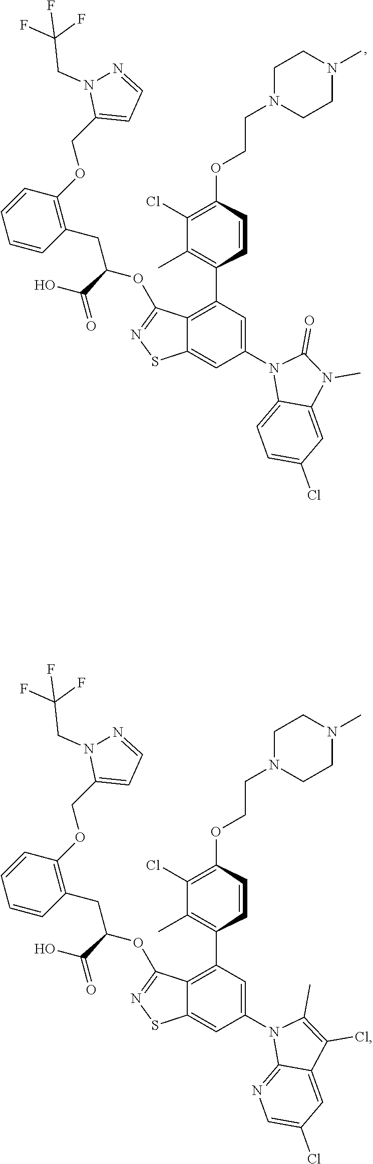

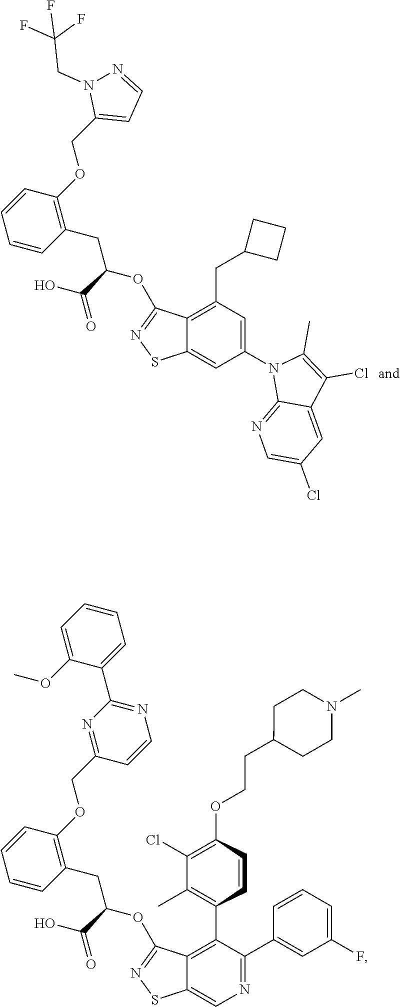

pharmaceutically acceptable salt thereof.

In some aspects, the invention relates to a compound of Formula II having a structure selected from:

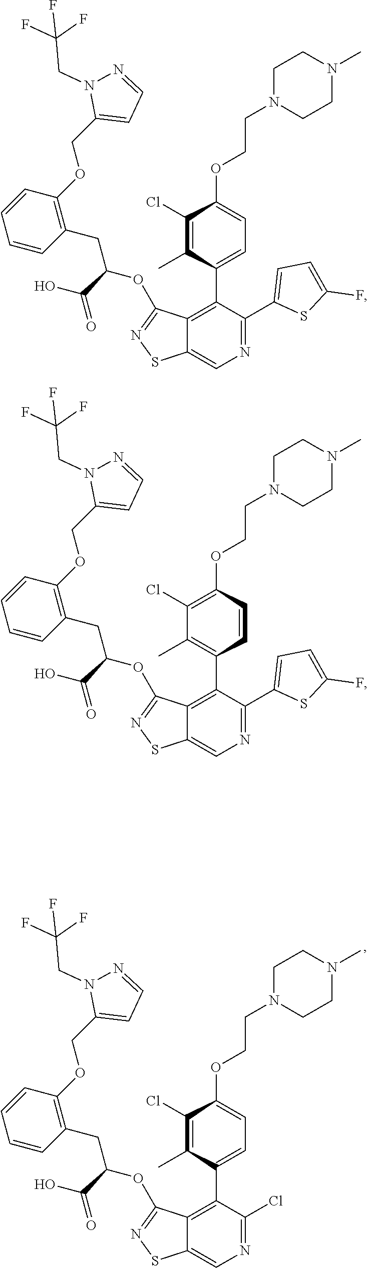

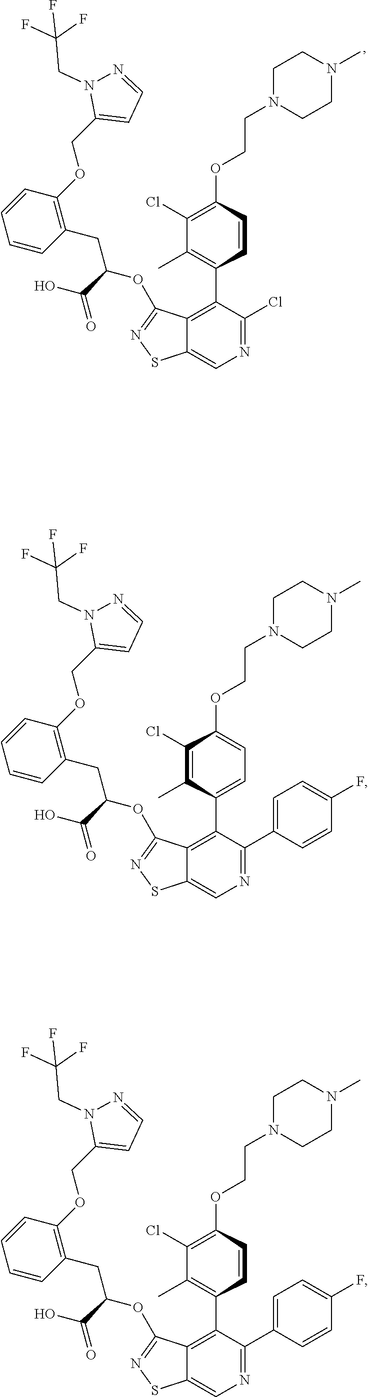

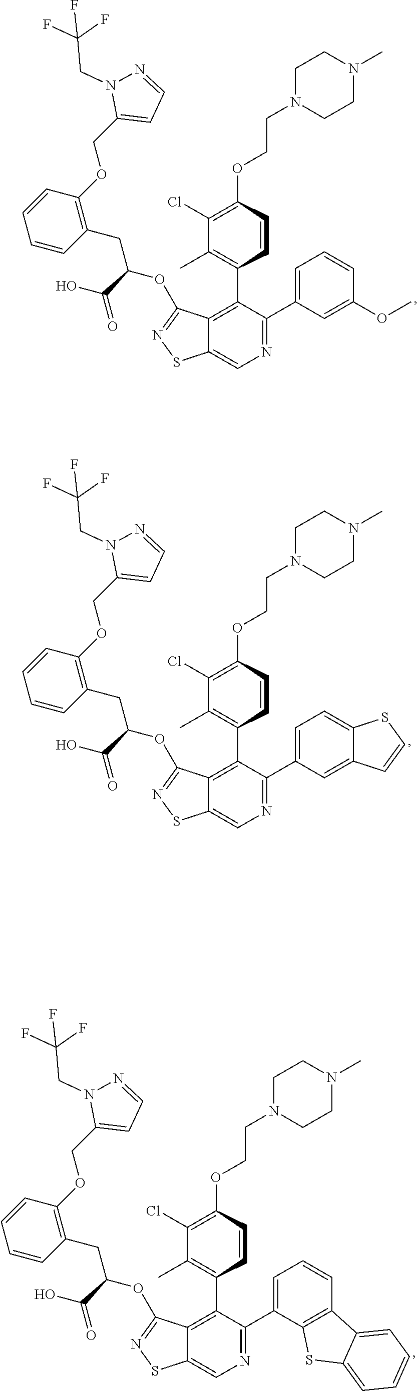

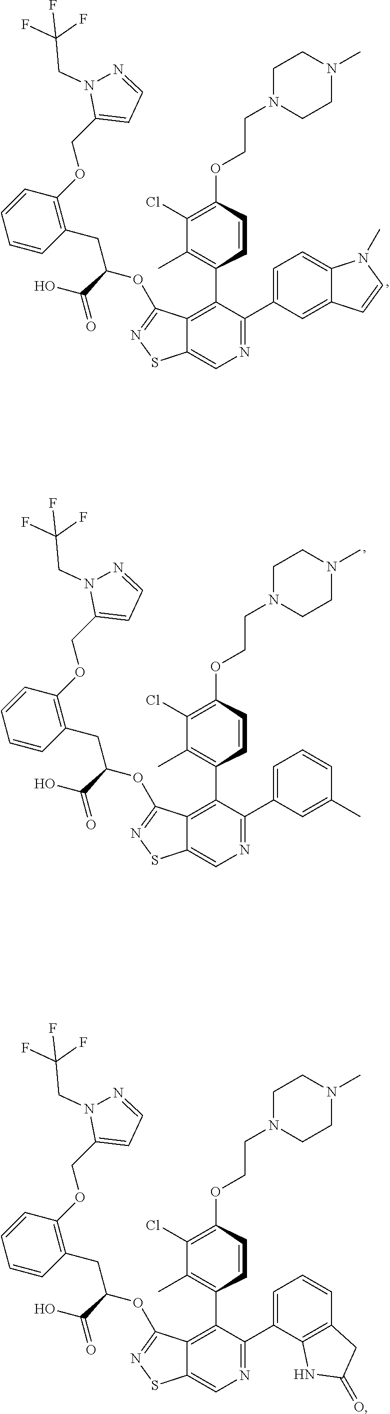

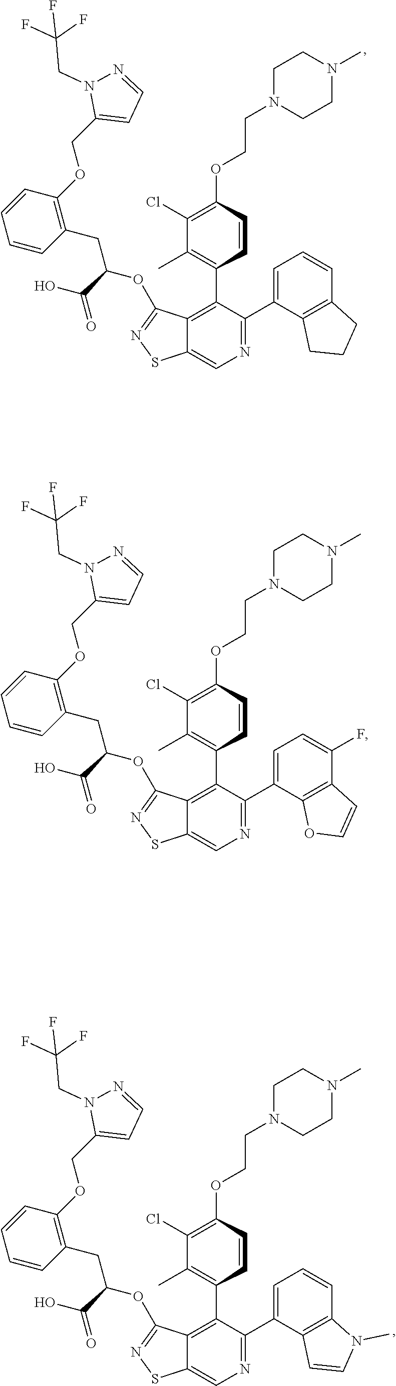

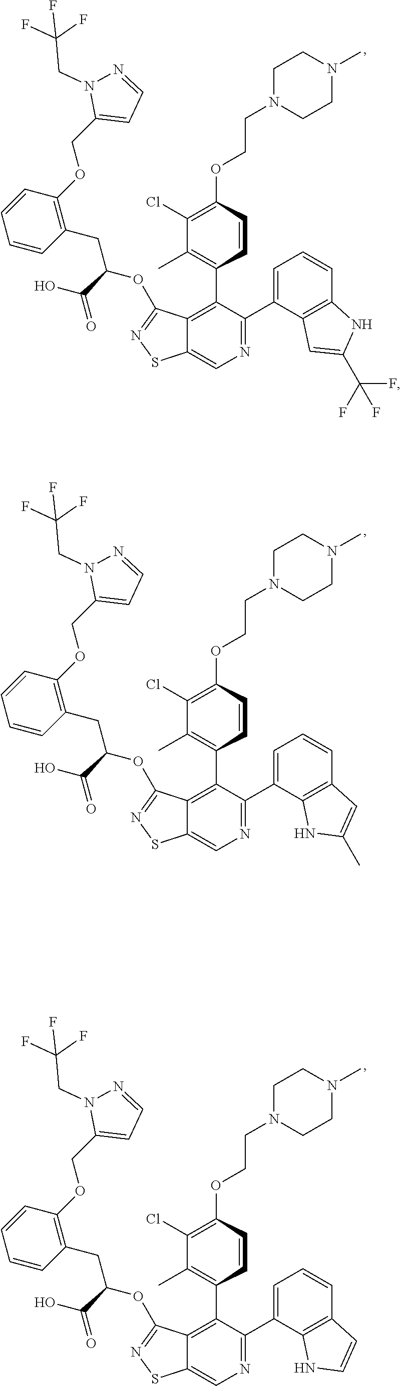

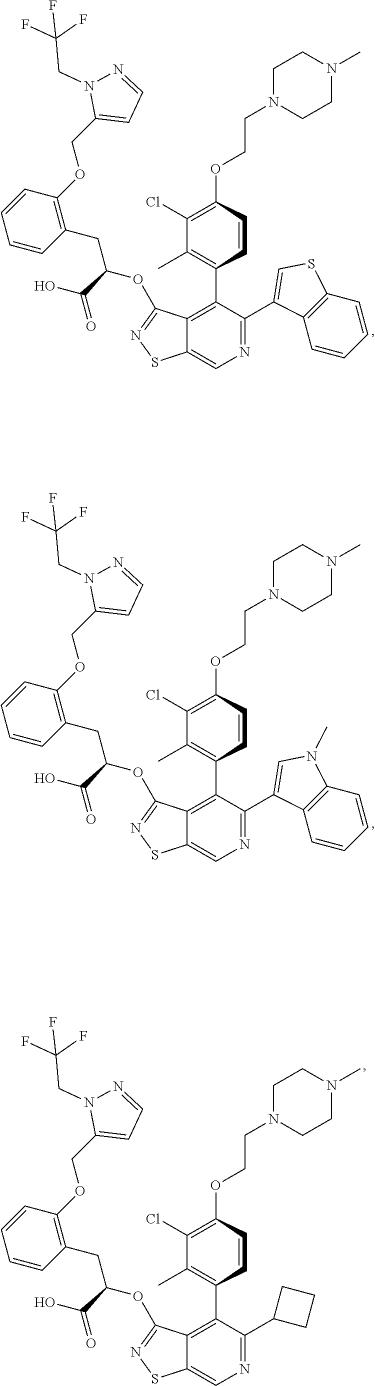

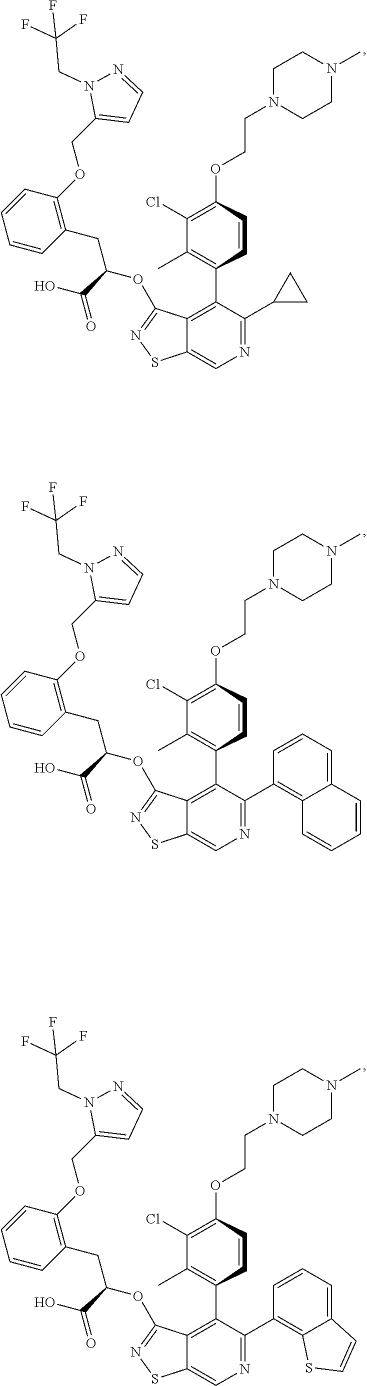

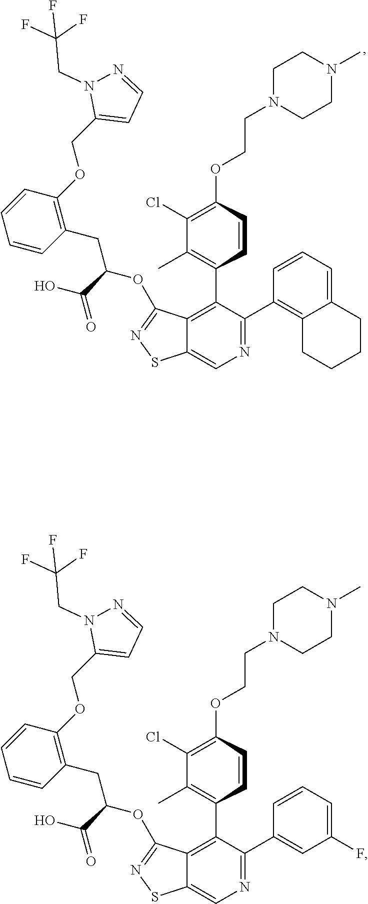

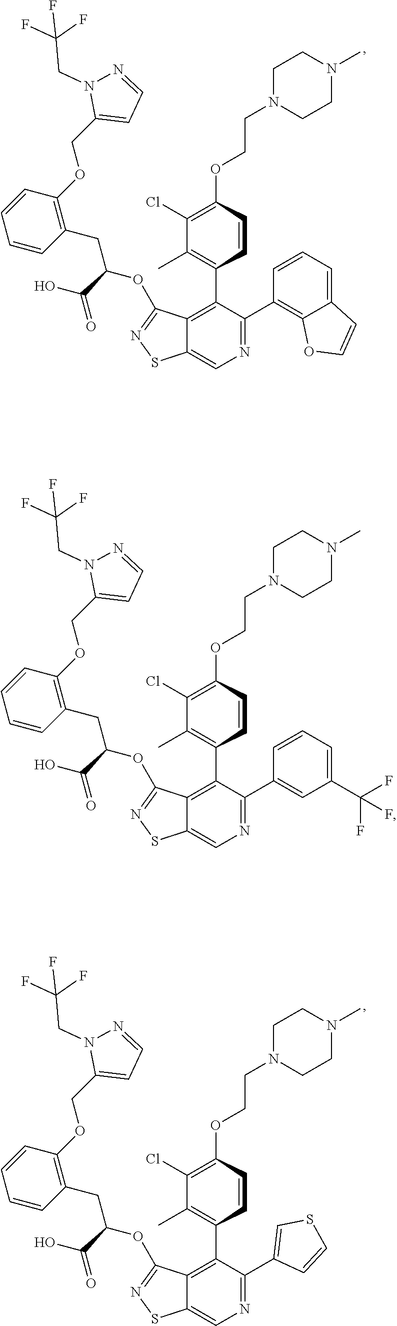

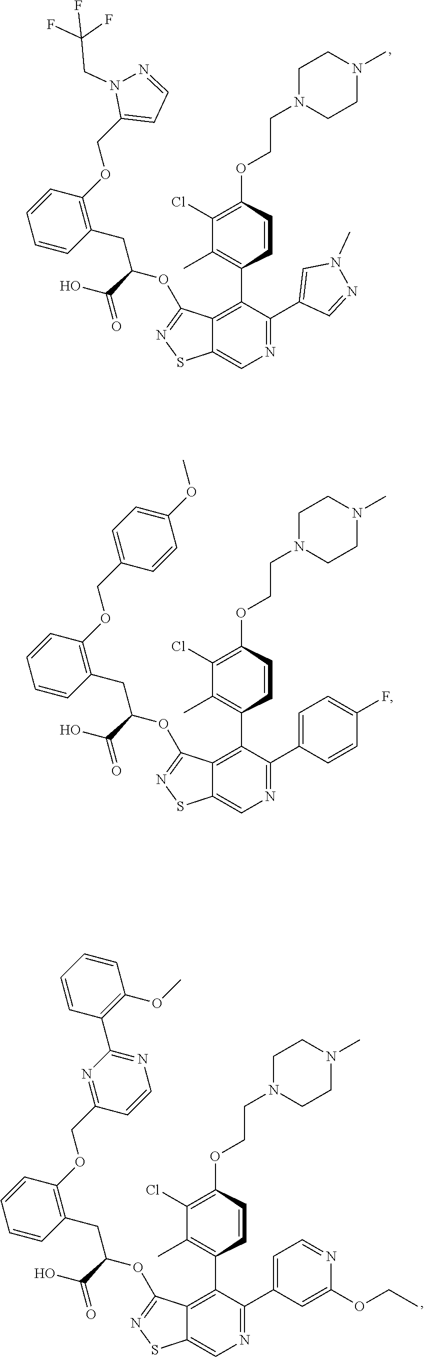

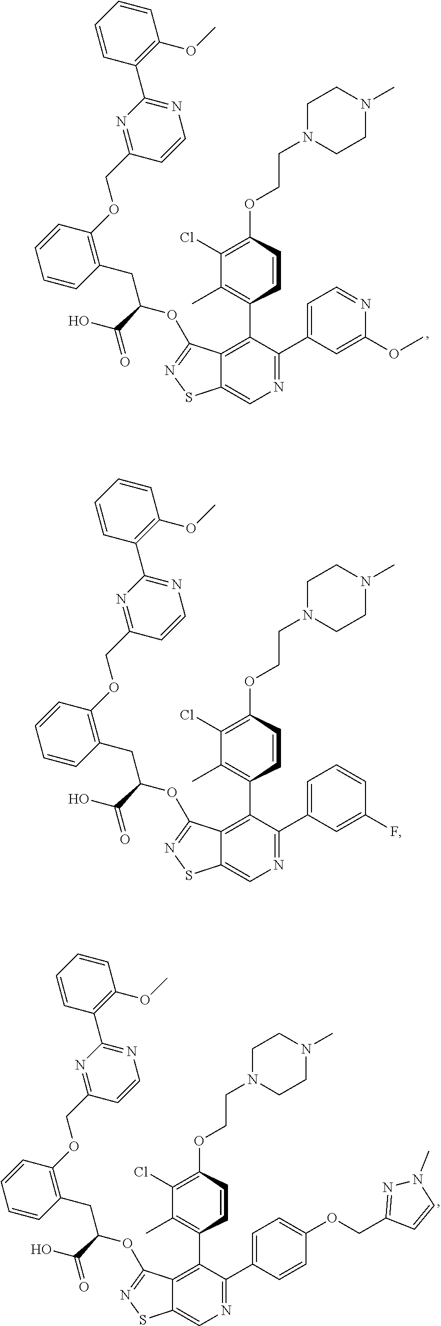

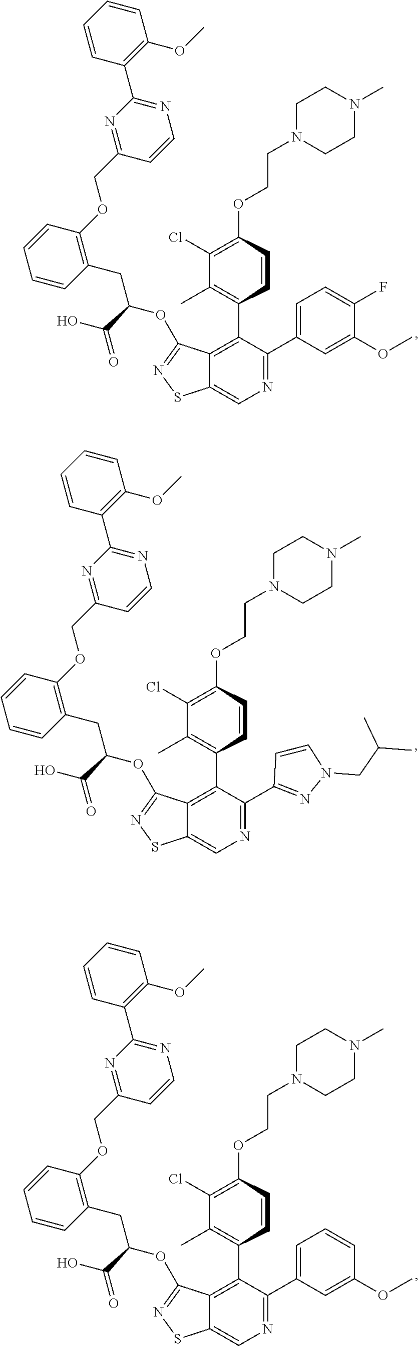

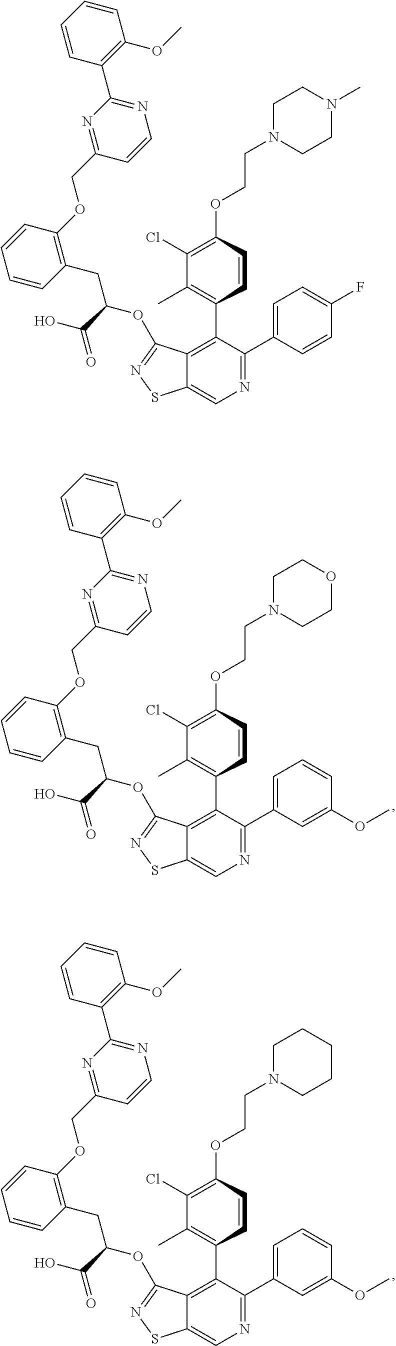

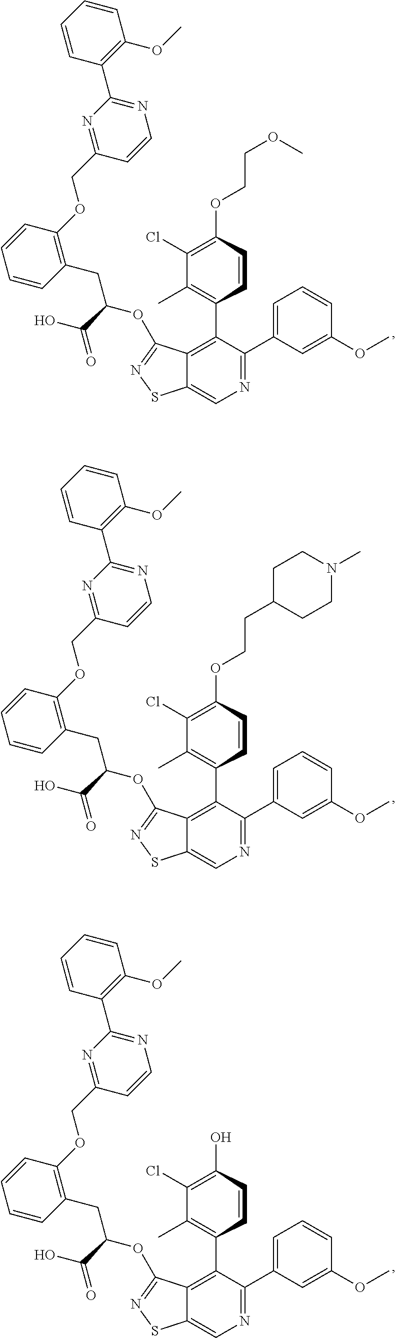

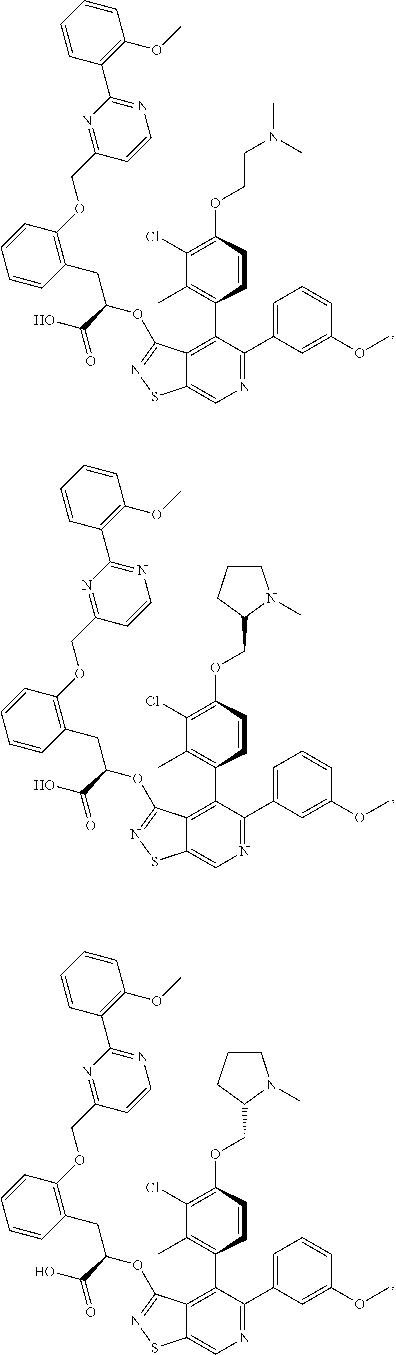

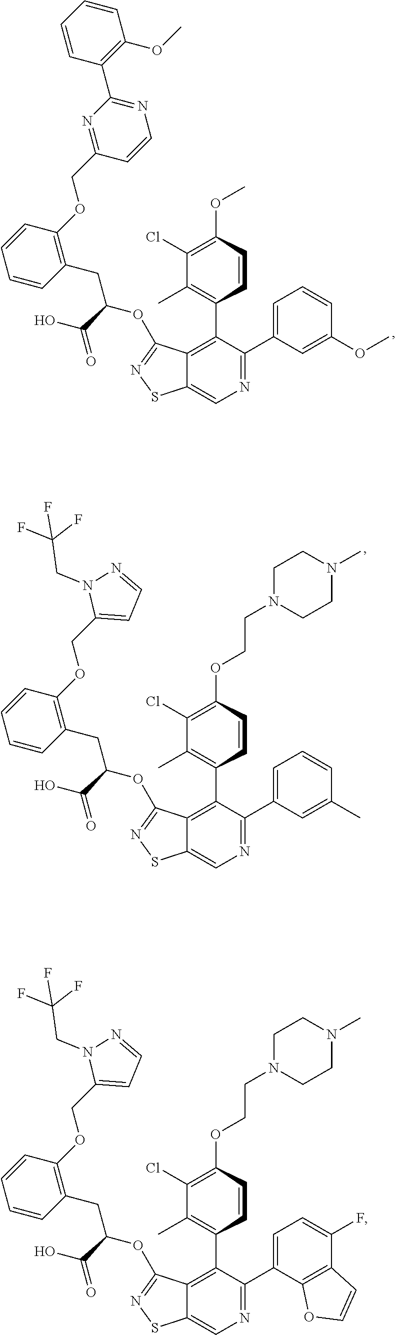

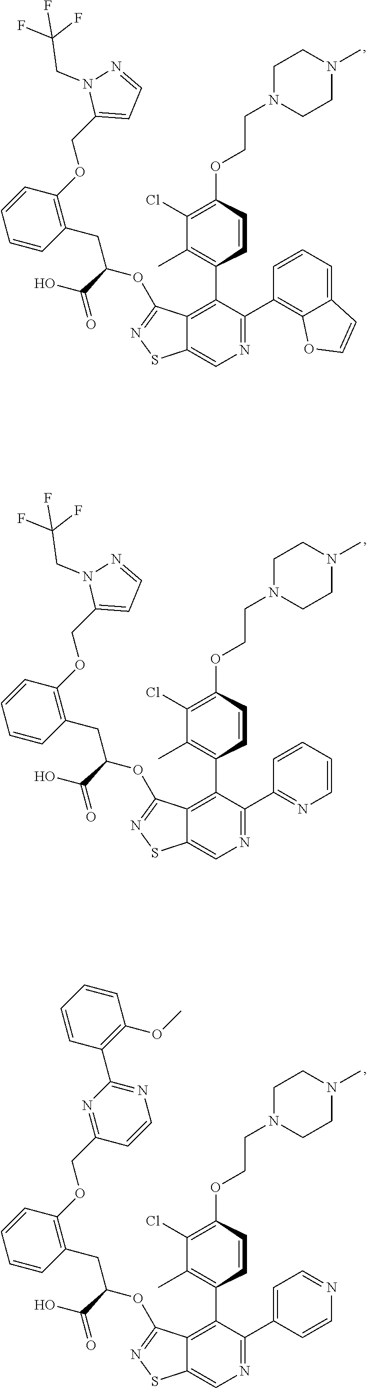

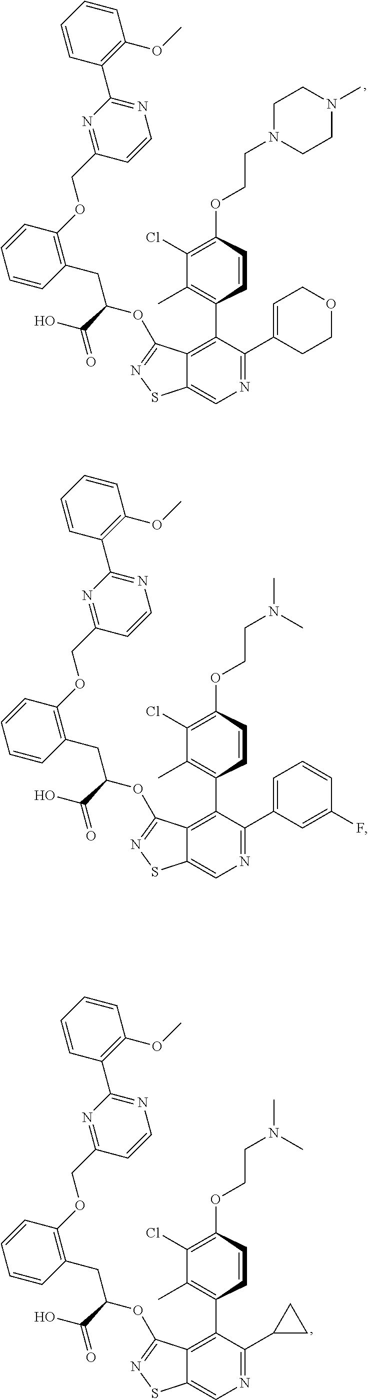

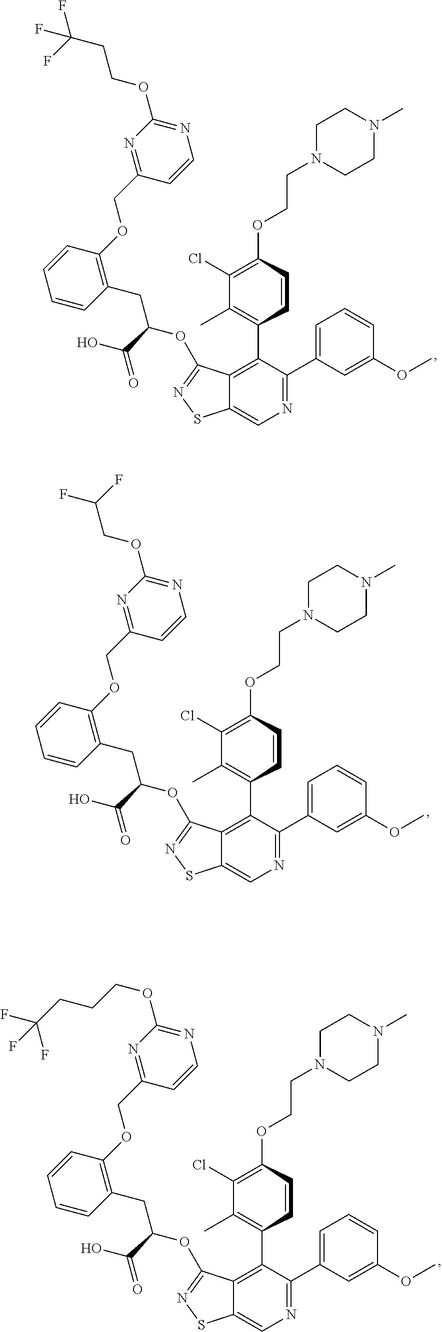

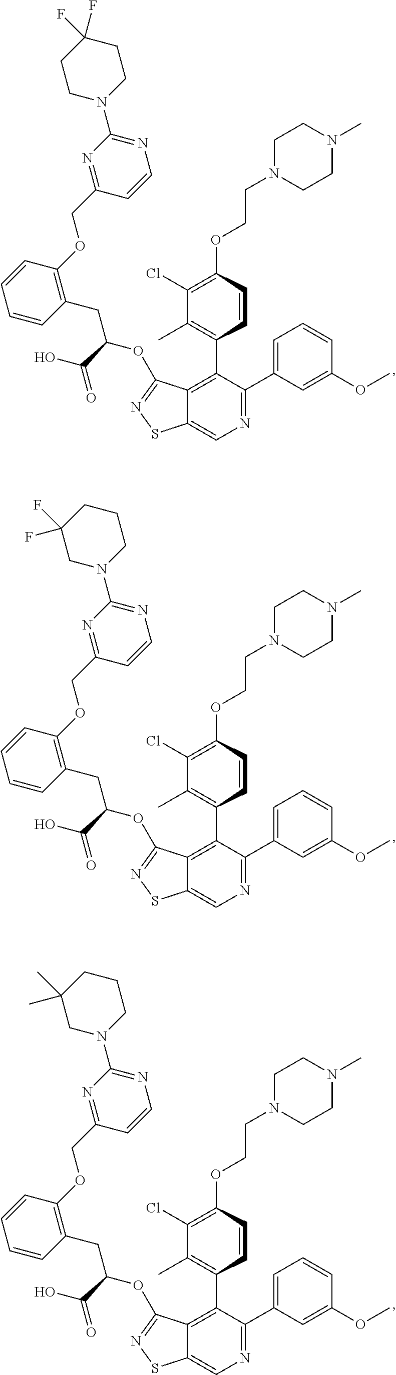

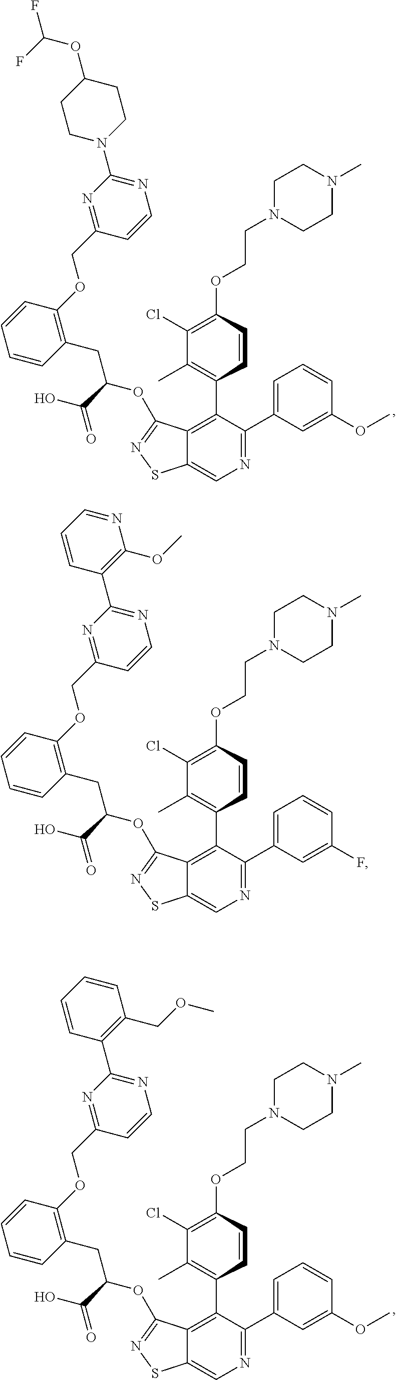

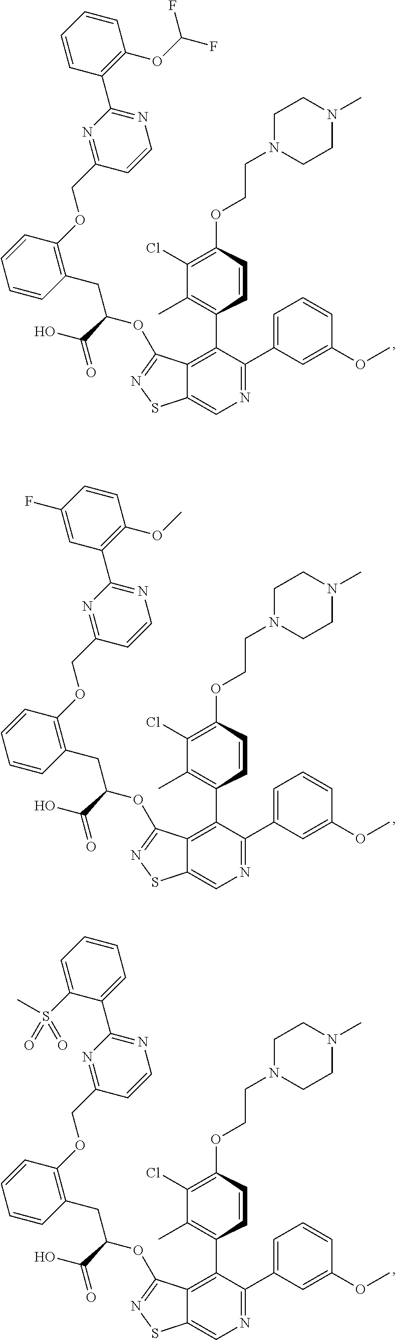

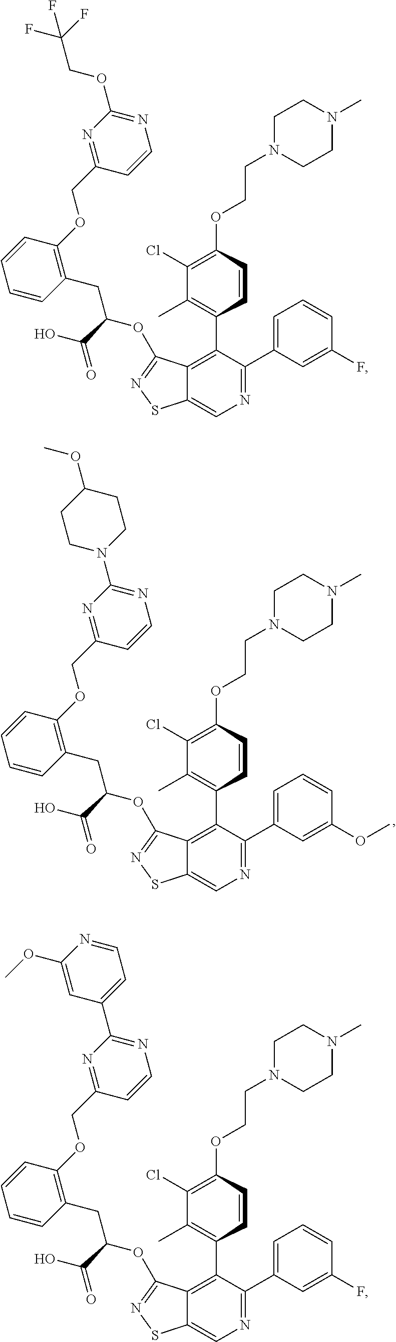

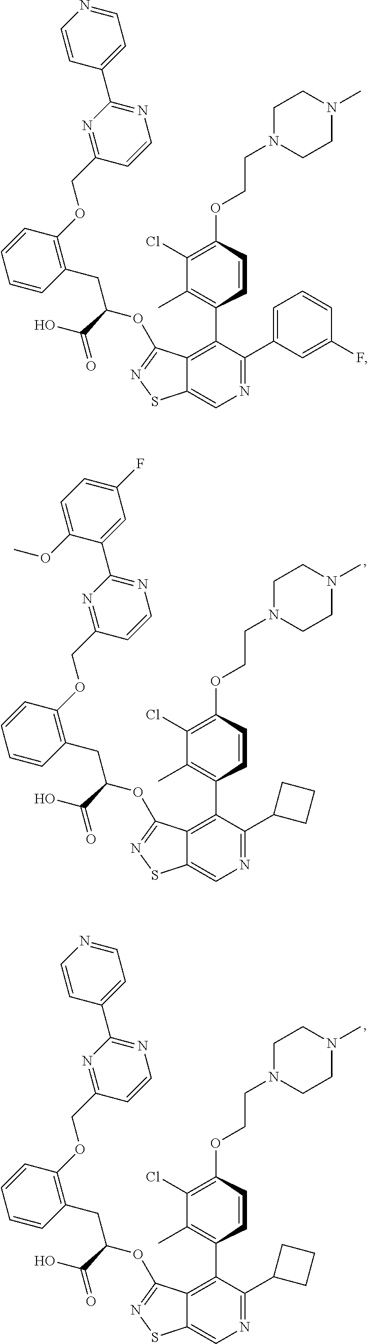

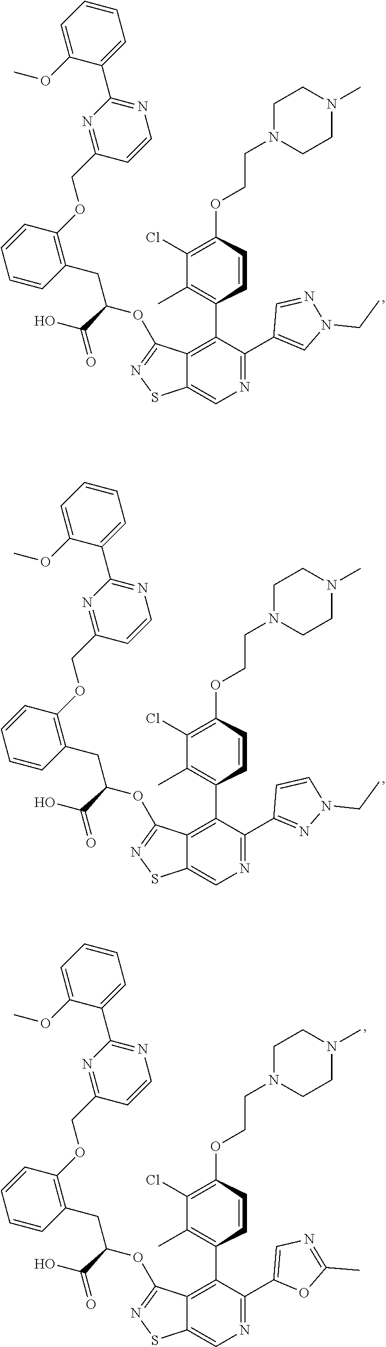

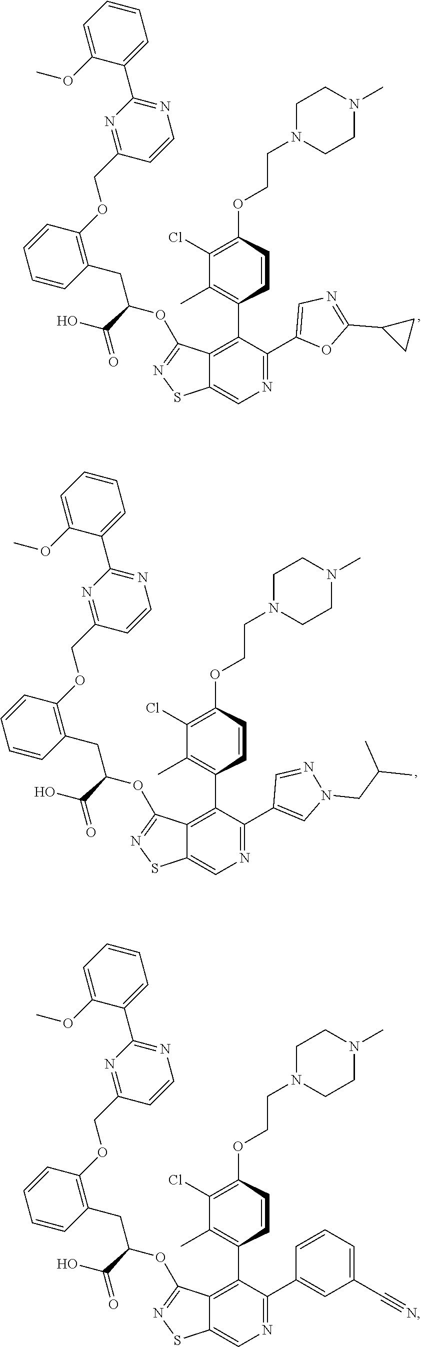

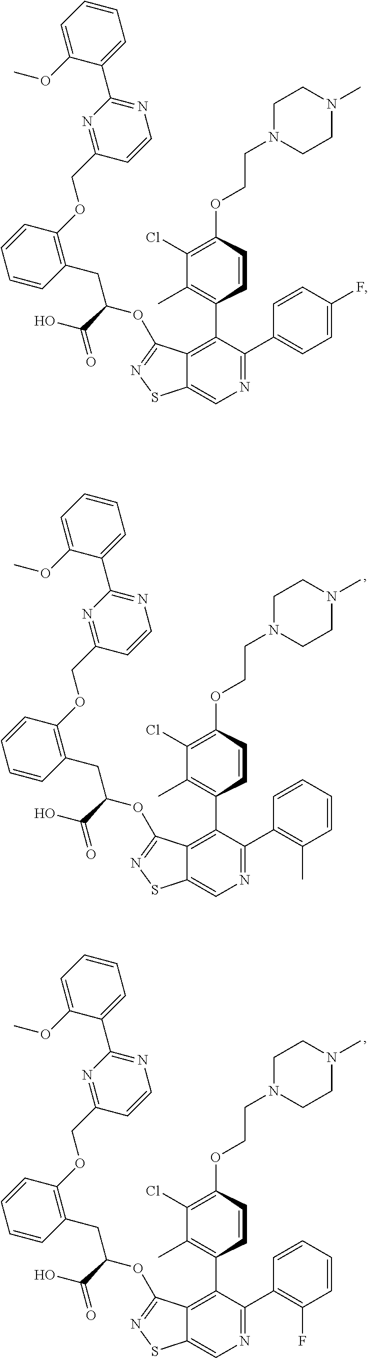

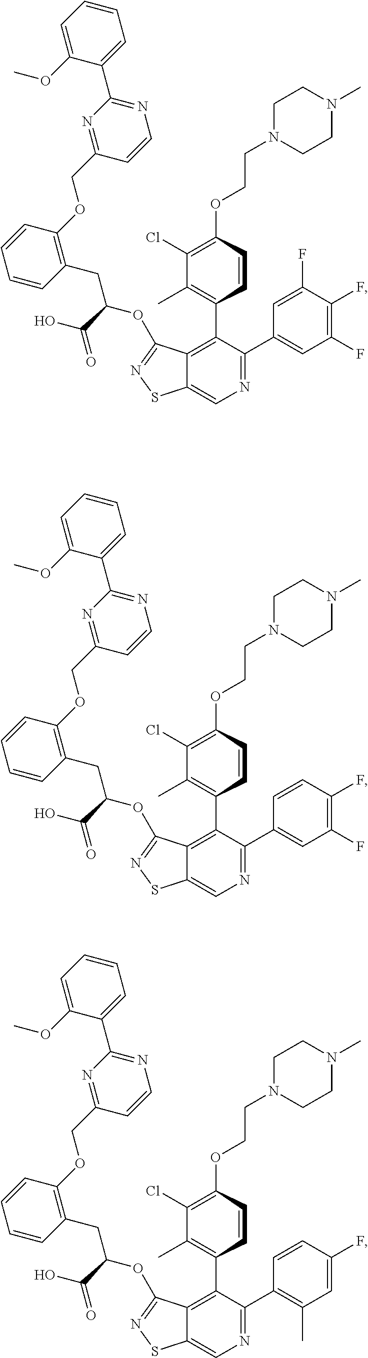

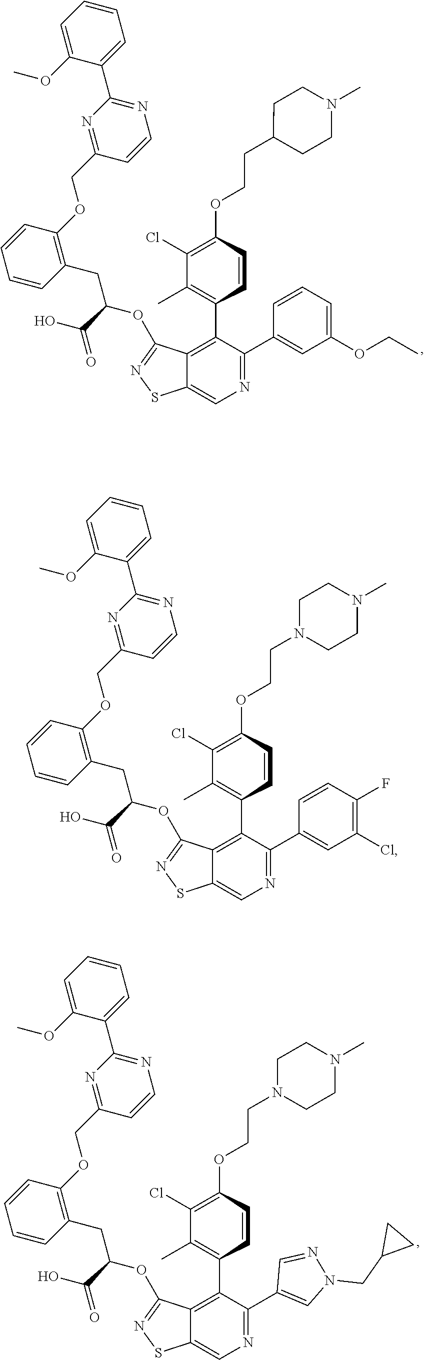

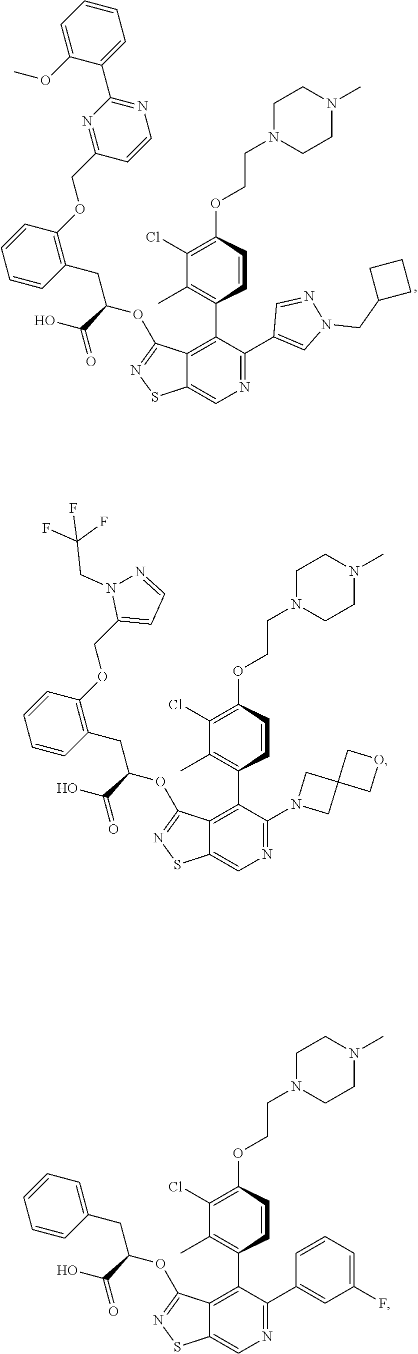

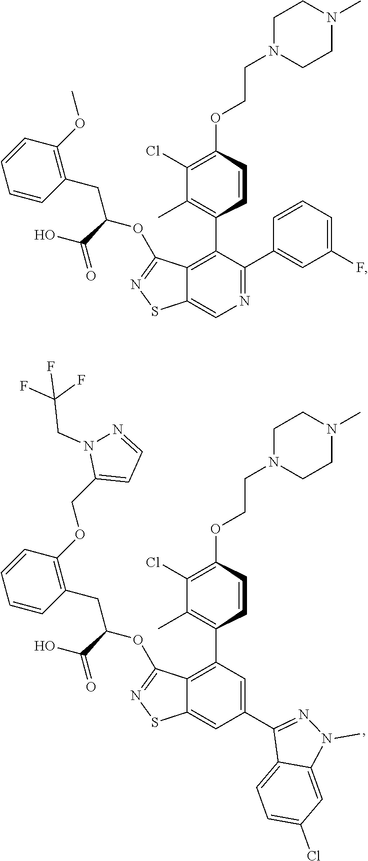

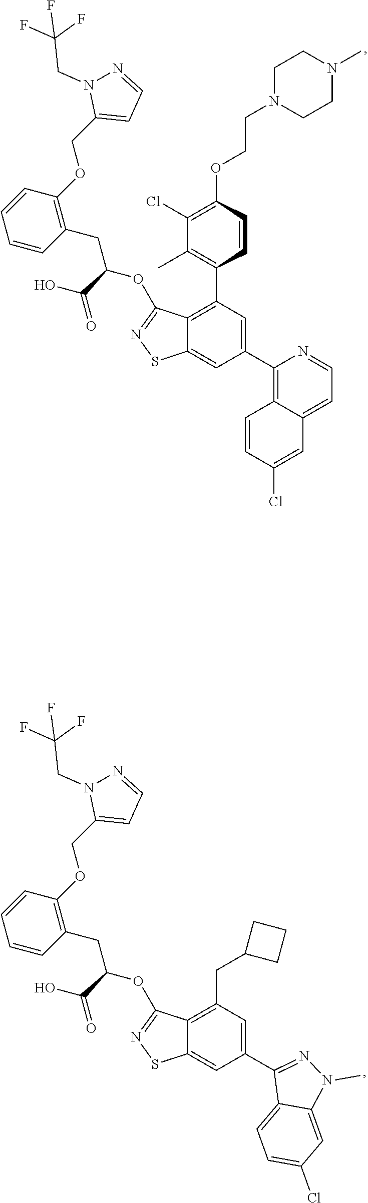

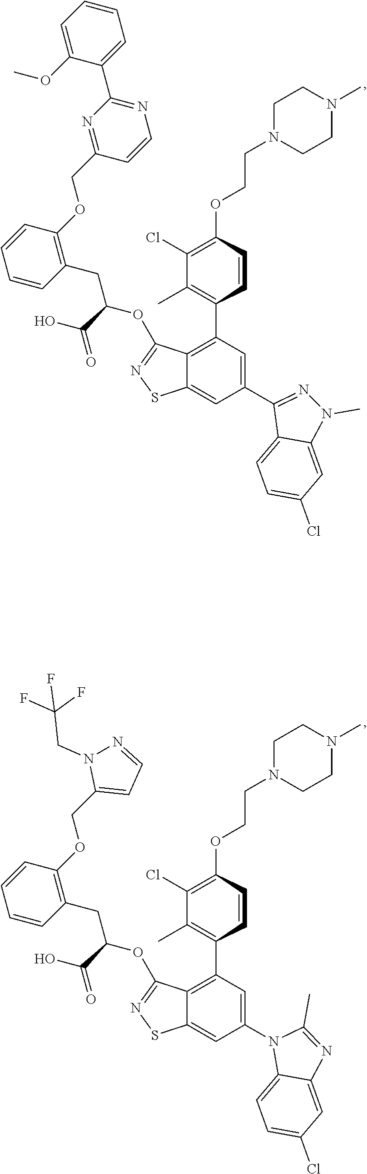

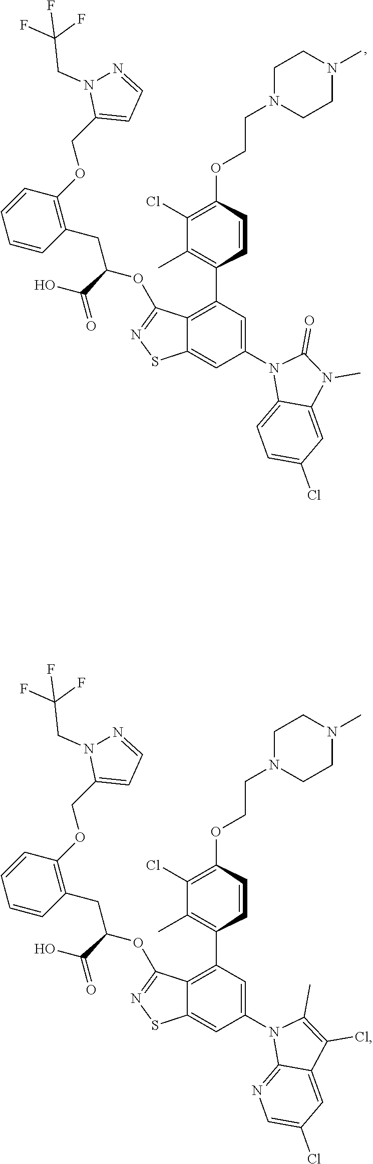

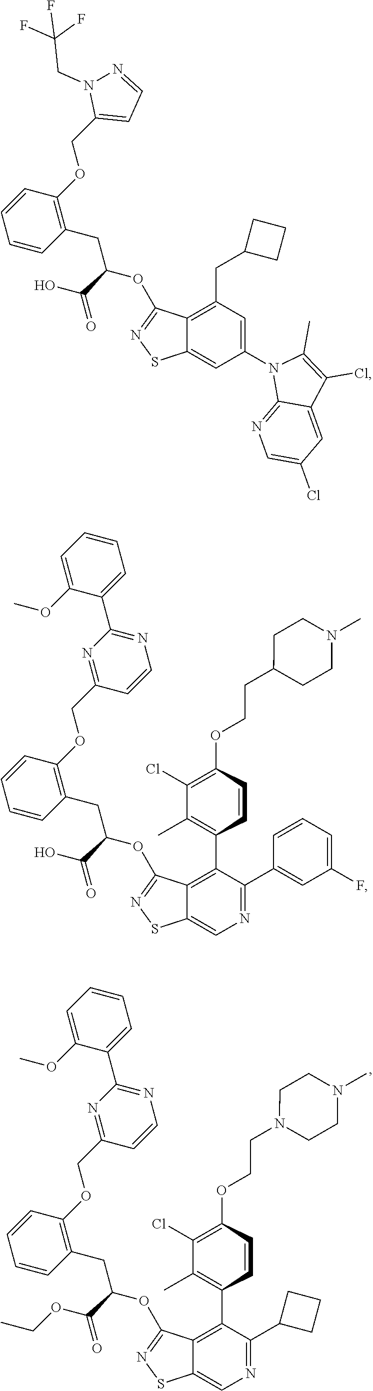

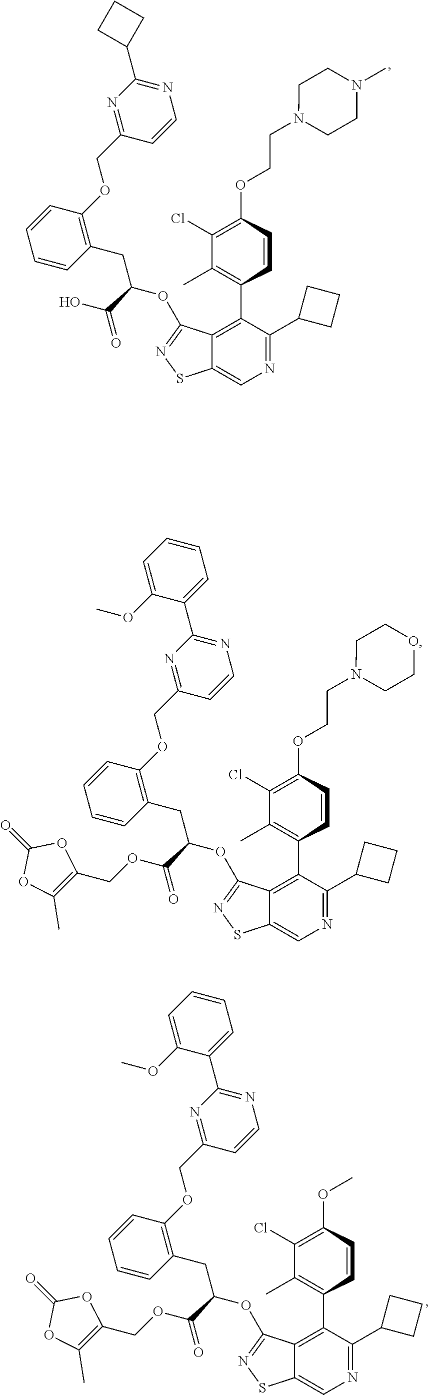

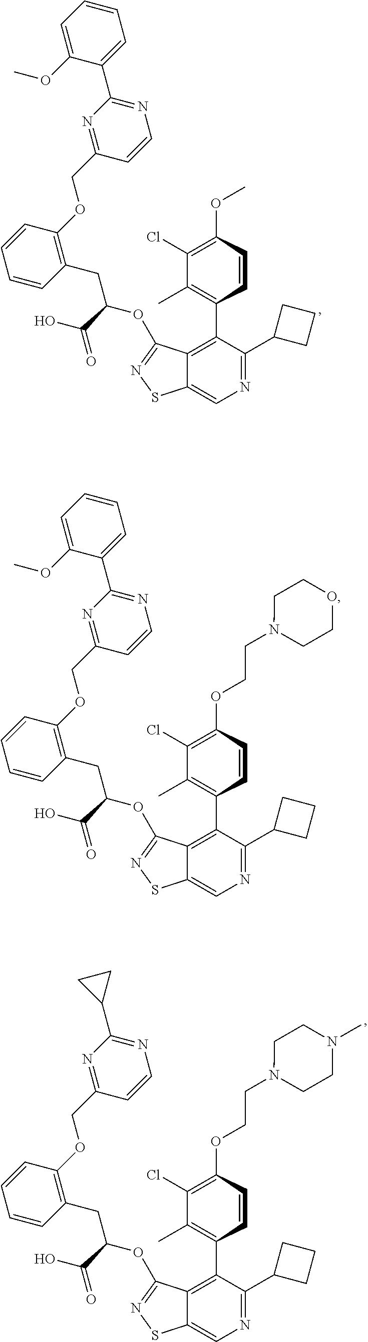

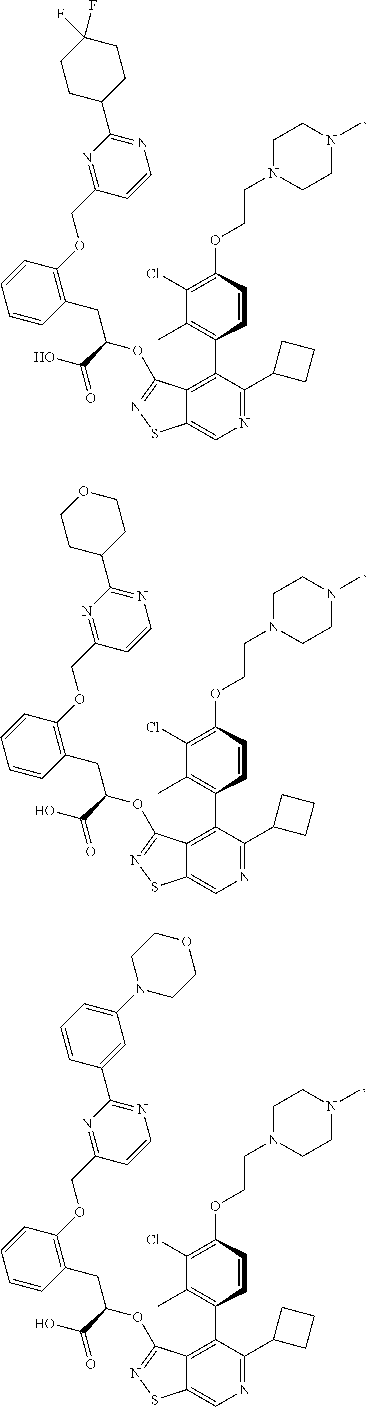

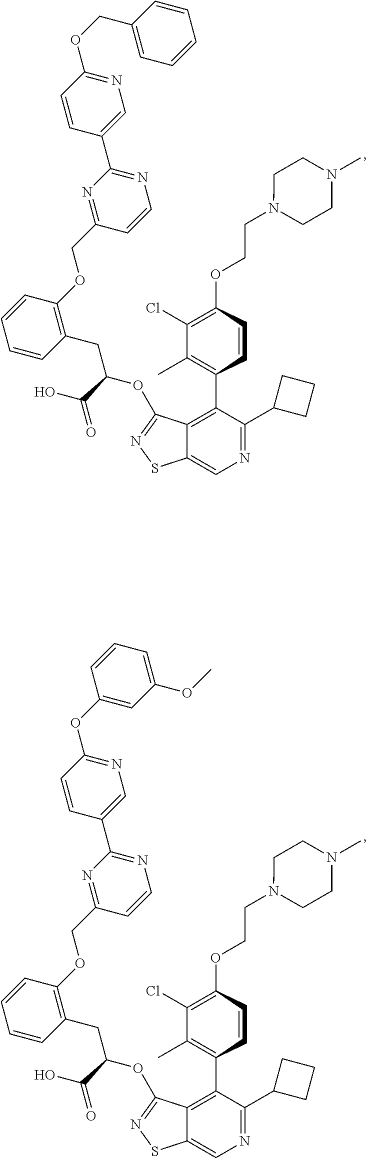

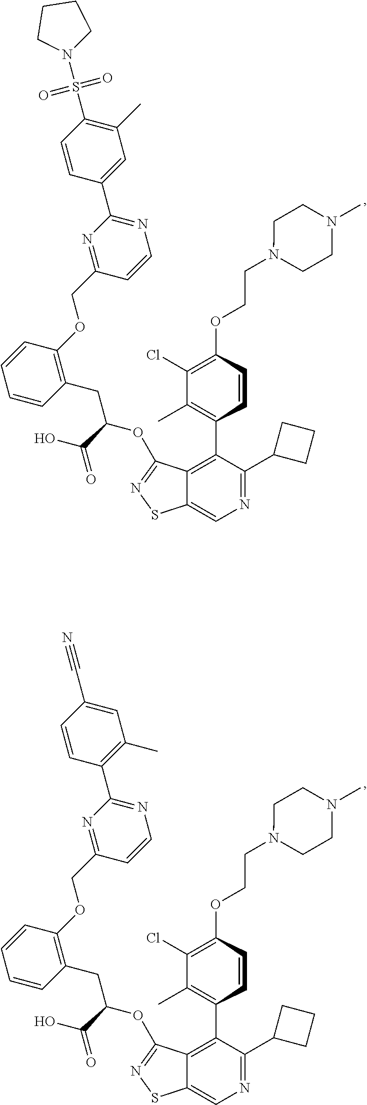

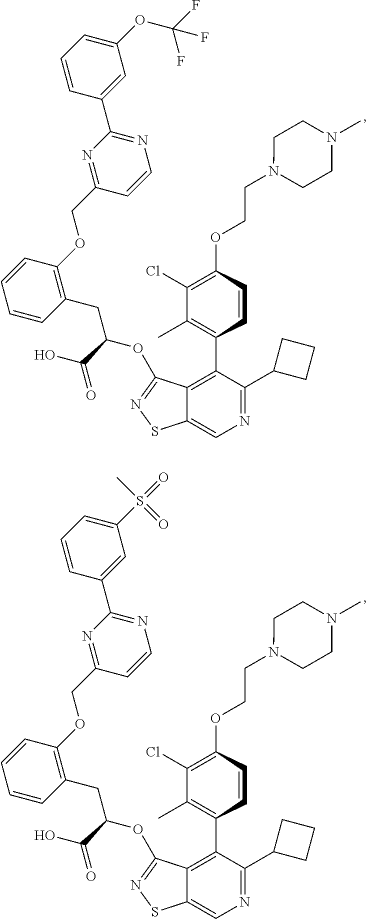

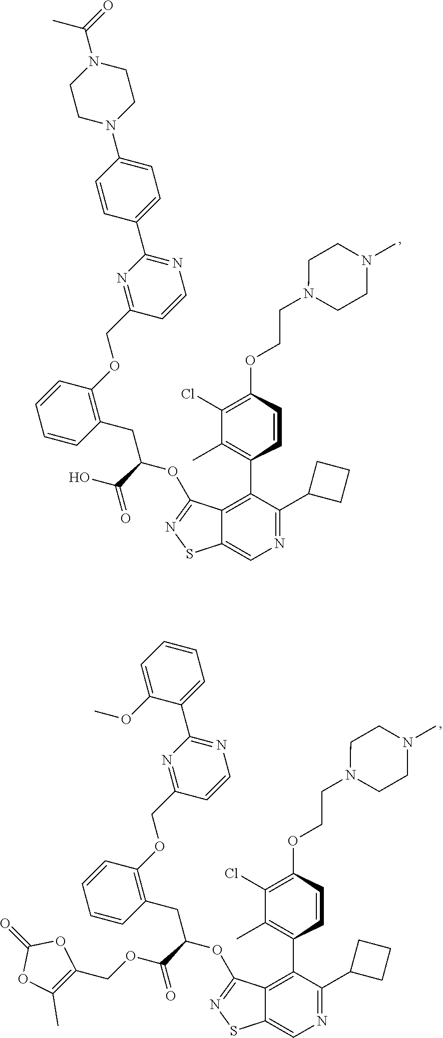

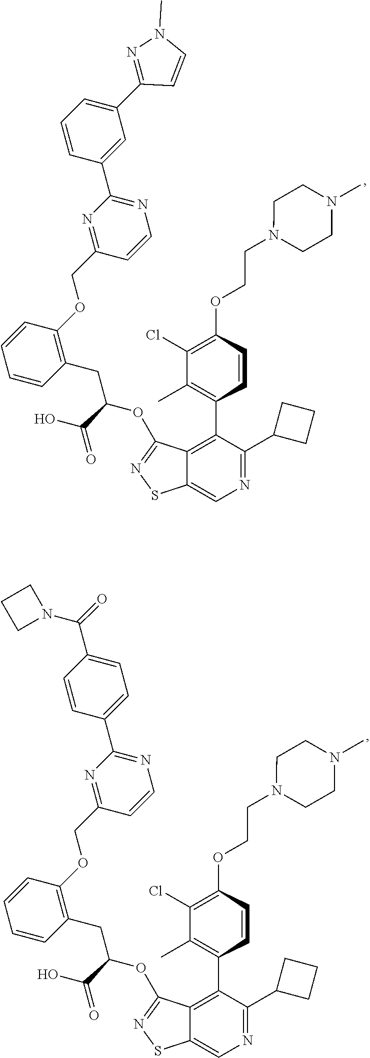

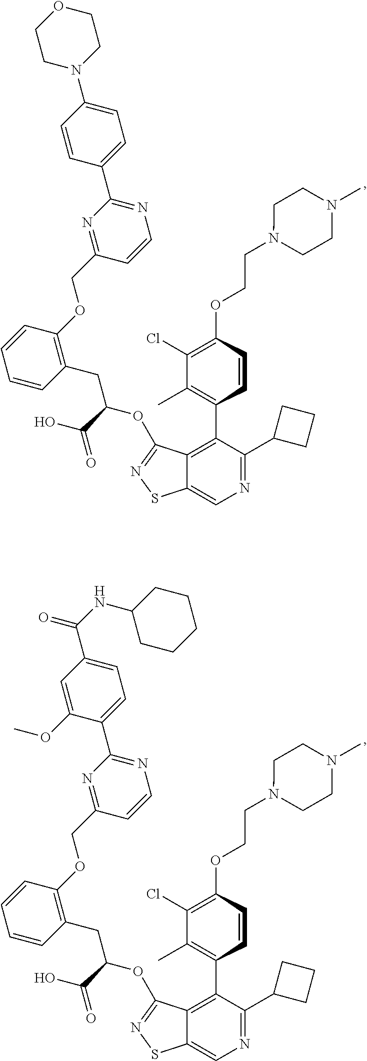

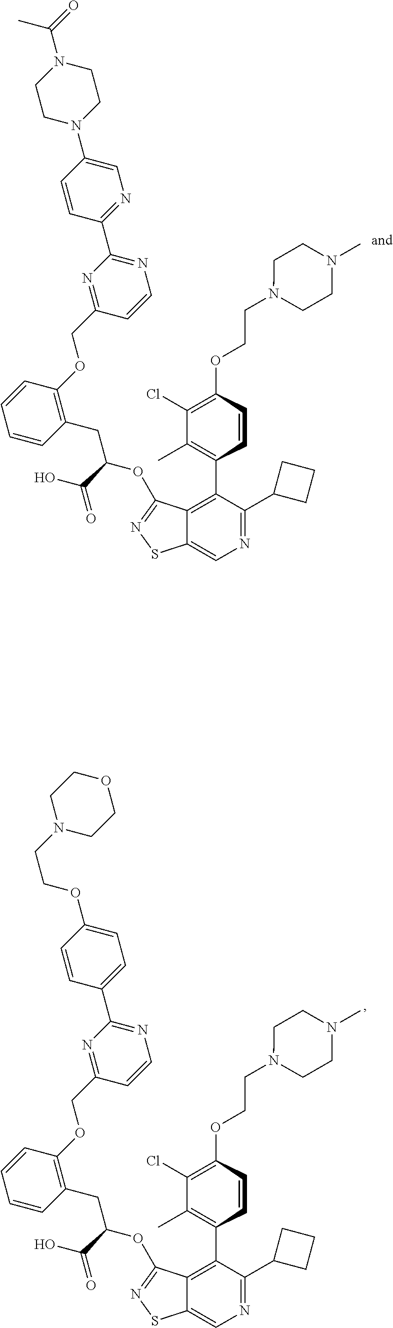

or a pharmaceutically acceptable salt thereof.

In some aspects, the invention relates to a compound of Formula II having a structure selected from:

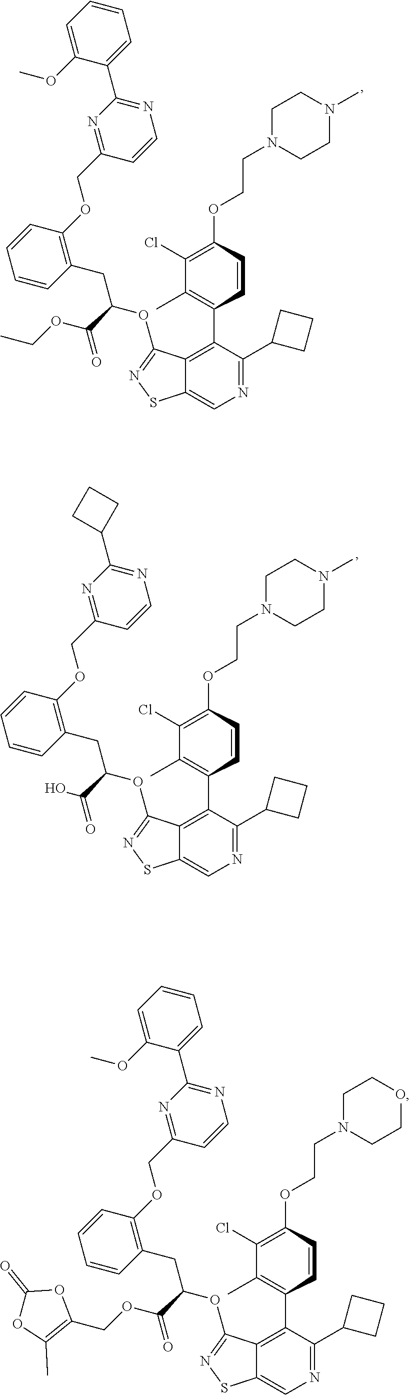

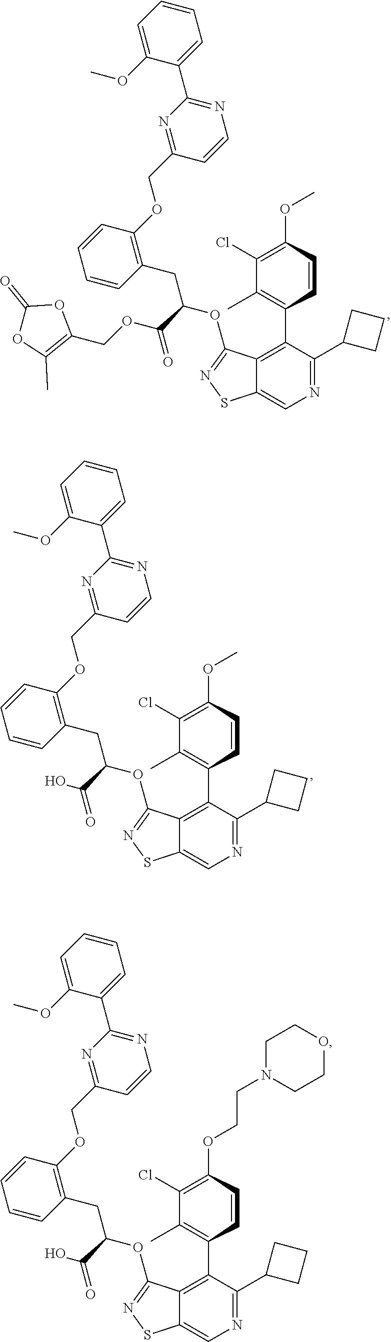

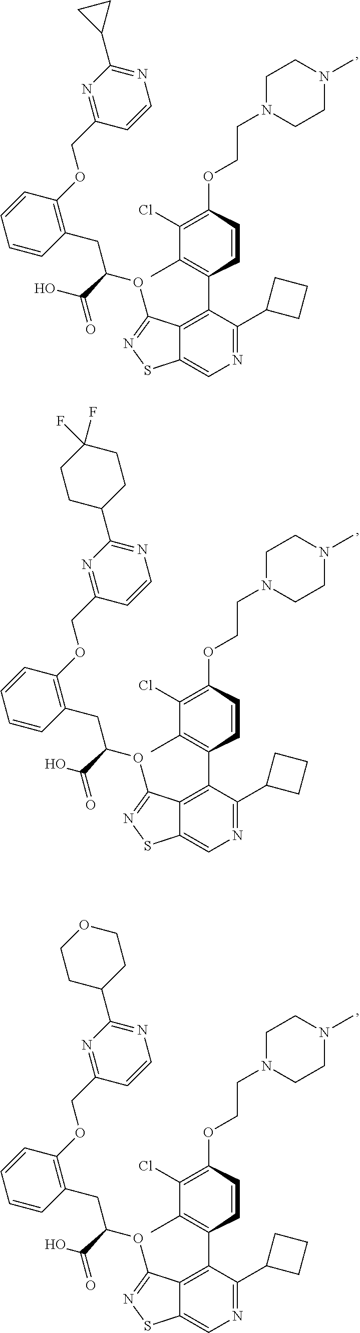

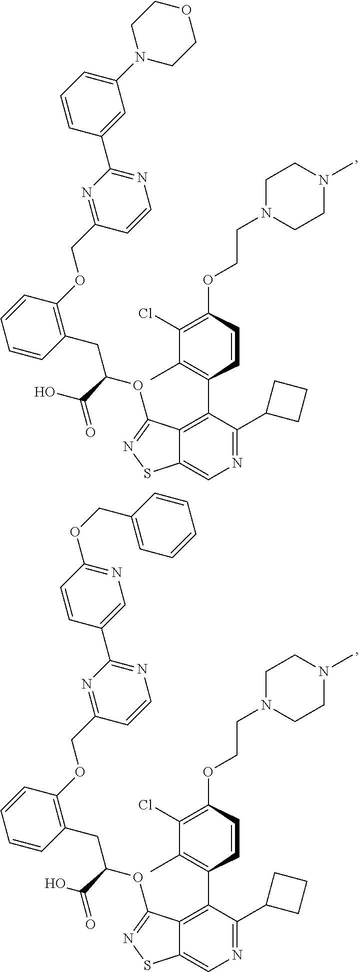

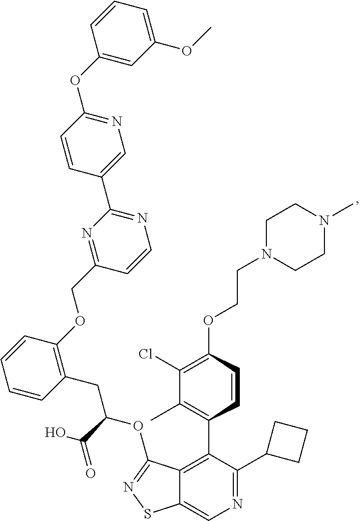

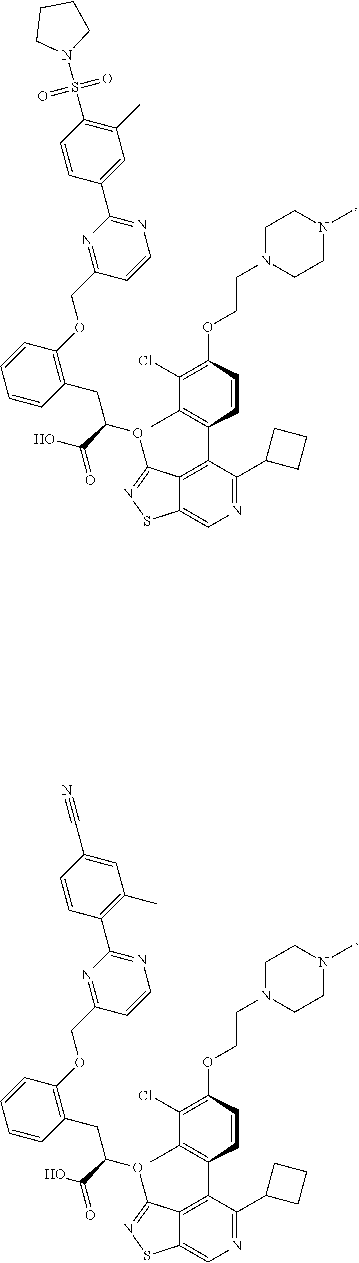

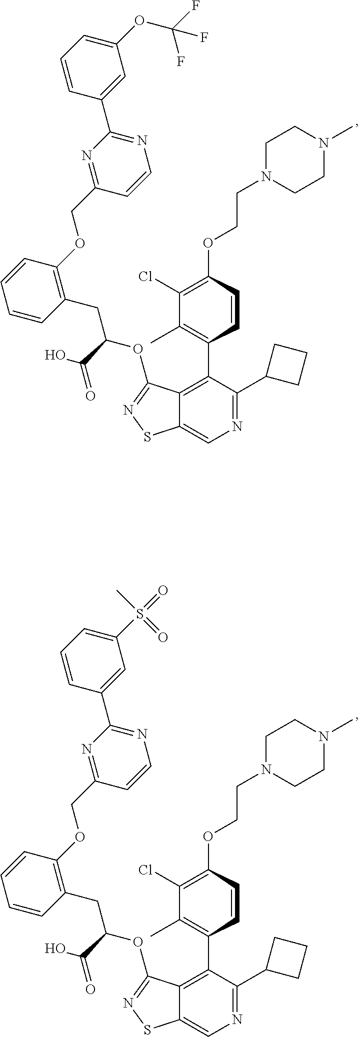

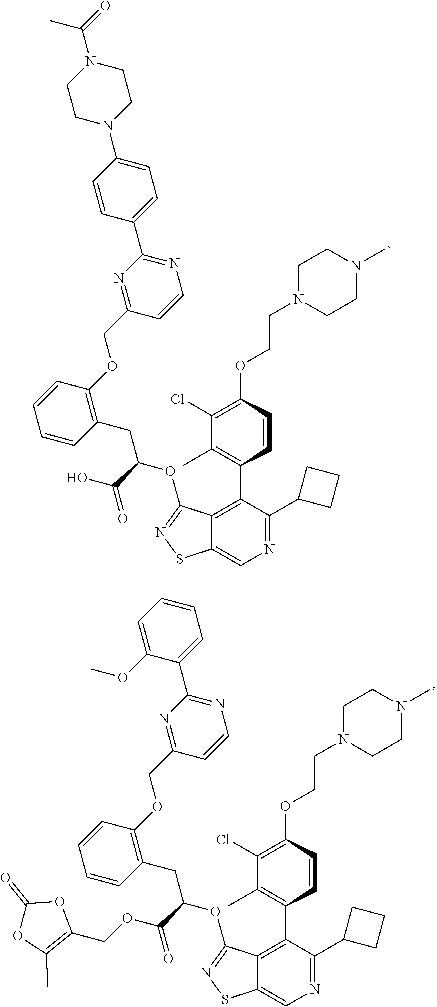

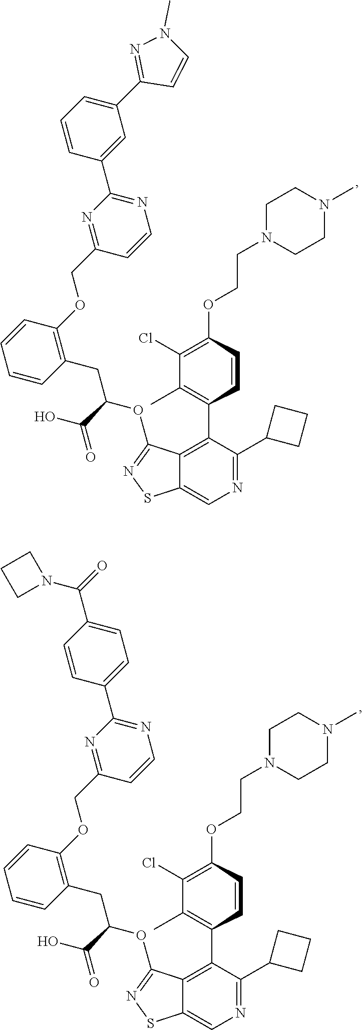

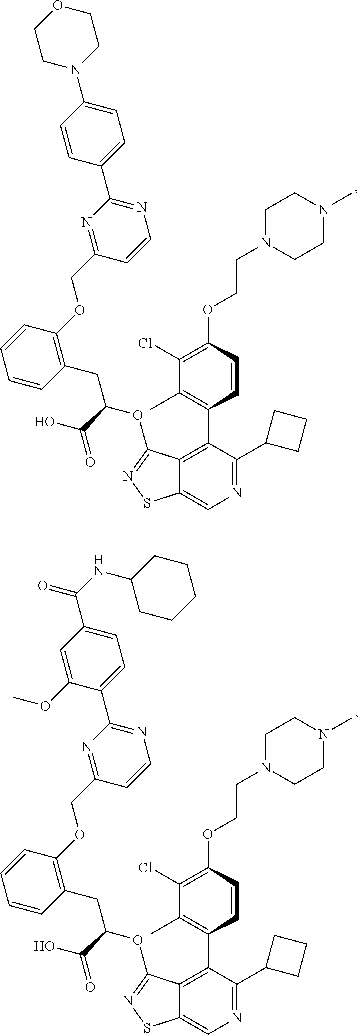

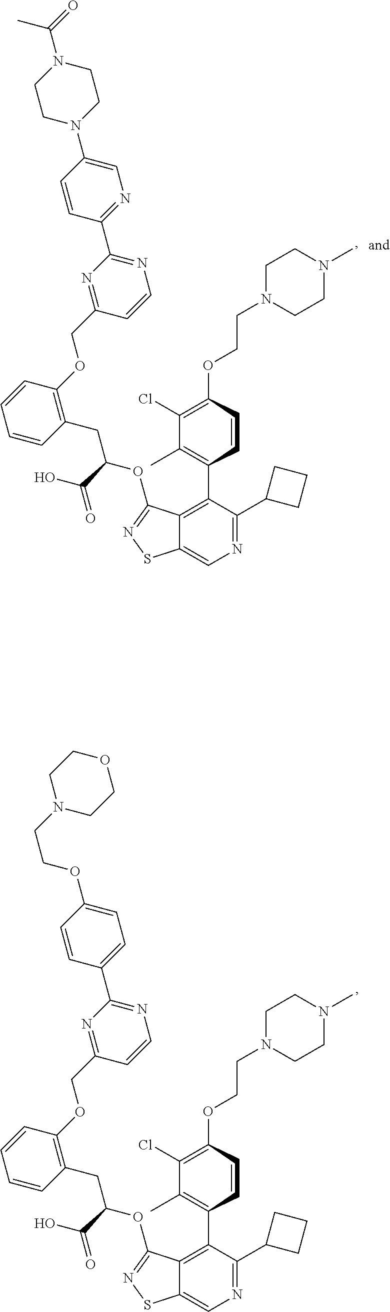

or a pharmaceutically acceptable salt thereof.

In some aspects, the invention relates to a compound of Formula II having a structure selected from:

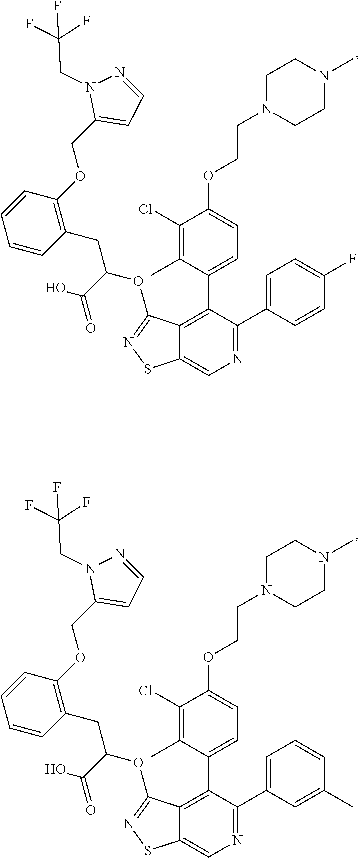 [SMILES: Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1]

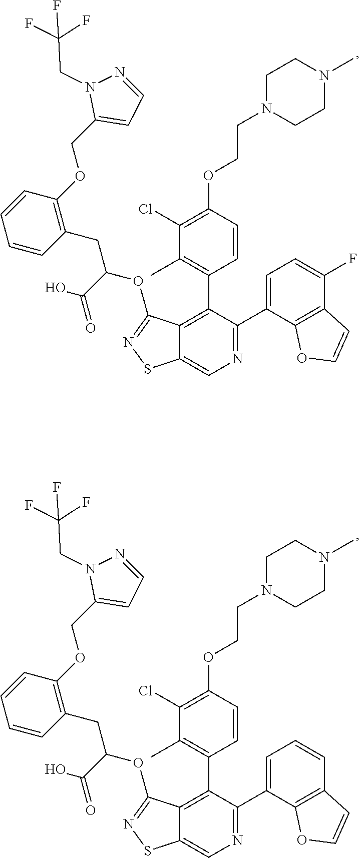 [SMILES: Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl]

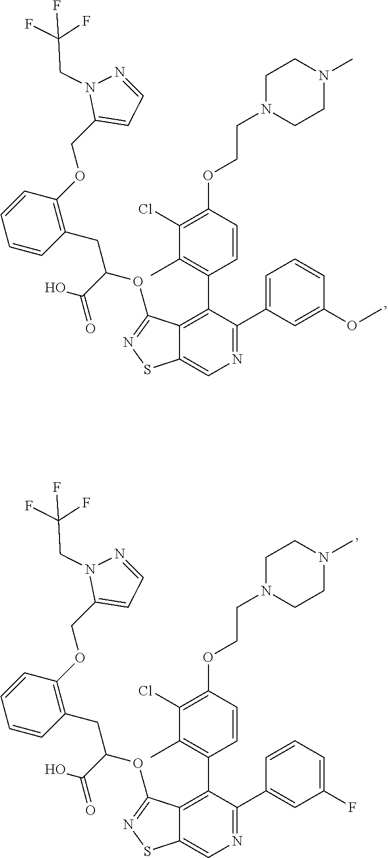 [SMILES: COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl]

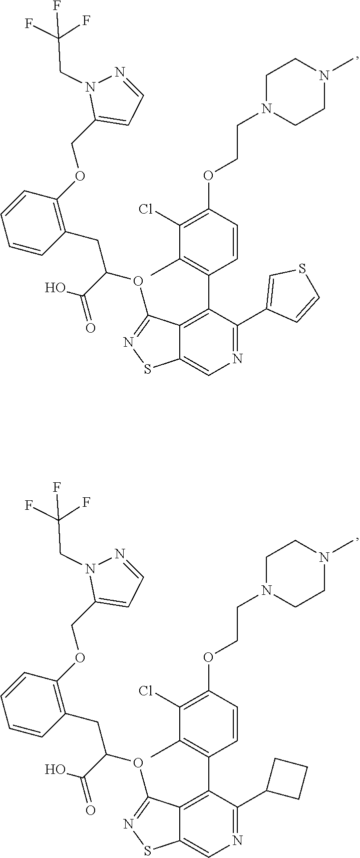 [SMILES: Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl]

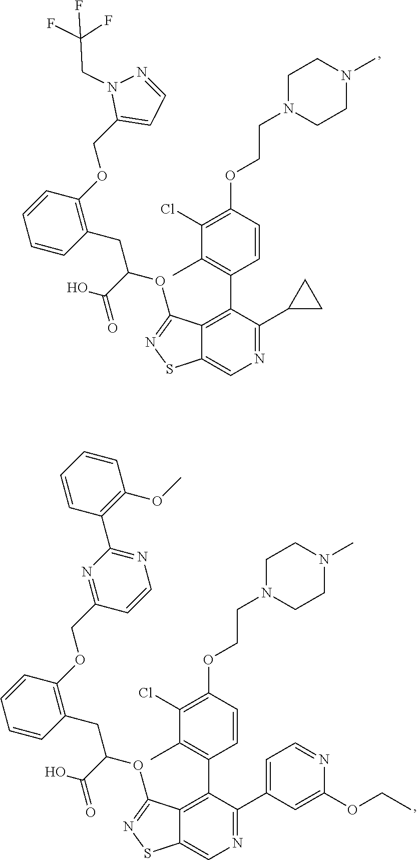 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1]

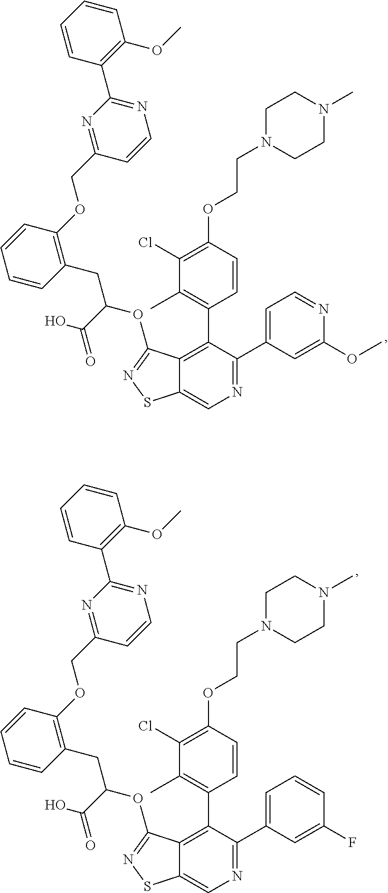 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1]

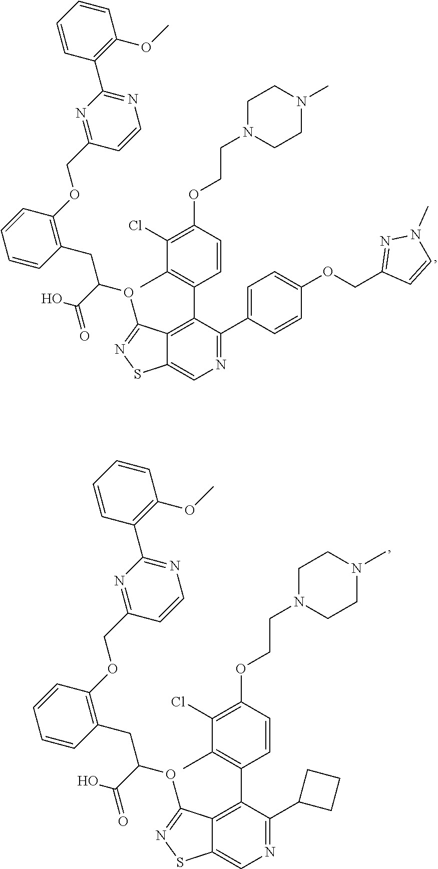 [SMILES: COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1]

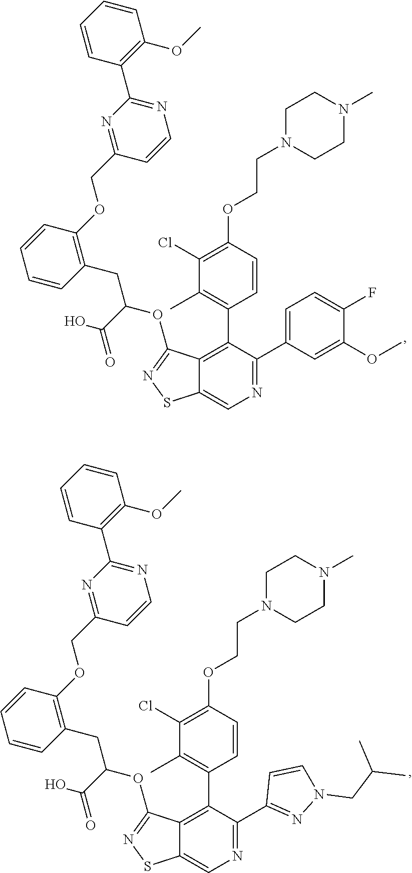 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F ; COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1]

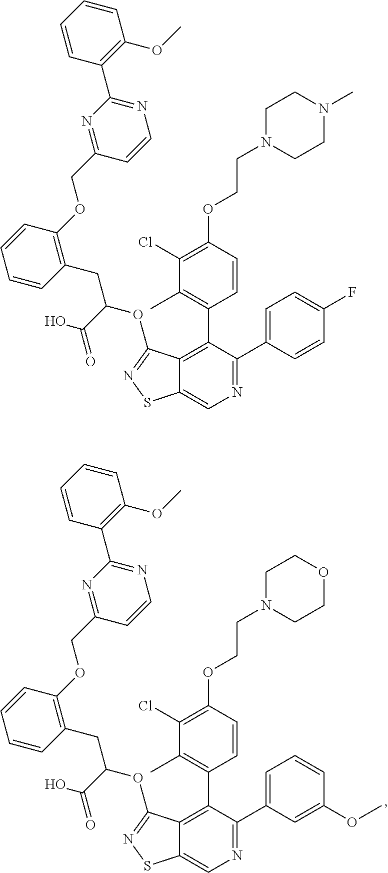 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1]

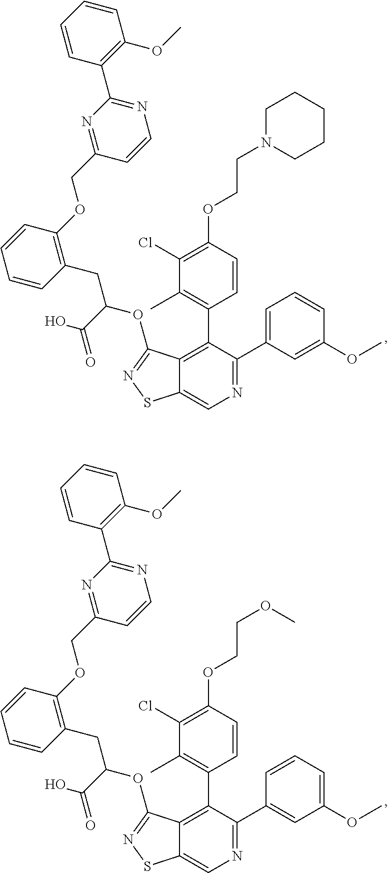 [SMILES: COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl]

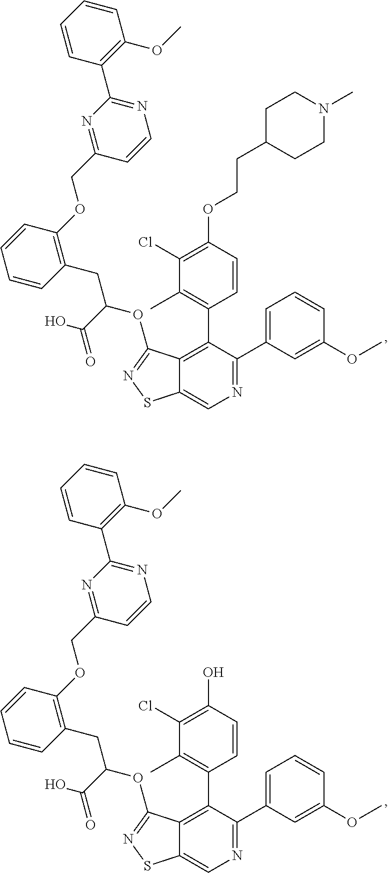 [SMILES: COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1]

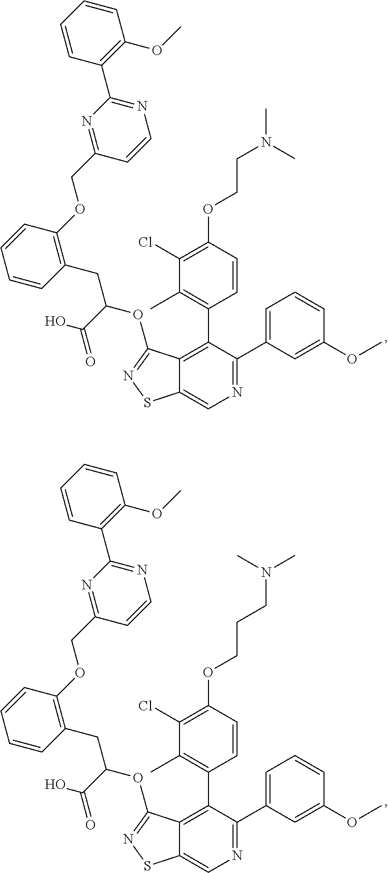 [SMILES: COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1]

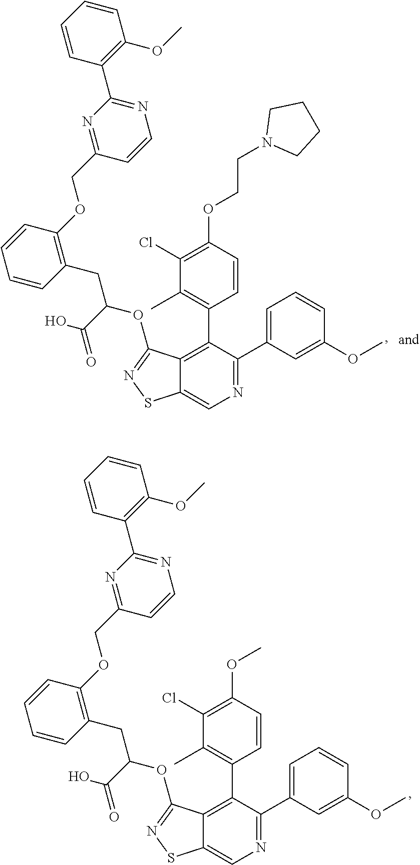 [SMILES: COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OC)c(Cl)c2C)c1]

or a pharmaceutically acceptable salt thereof.

In other aspects, the invention relates to a compound of Formula II having a structure selected from:

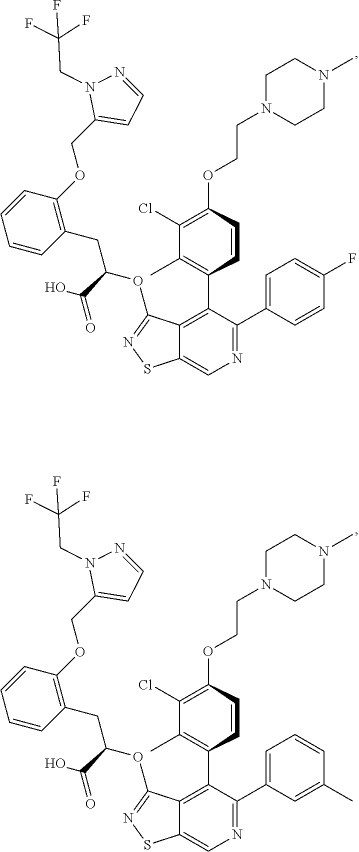

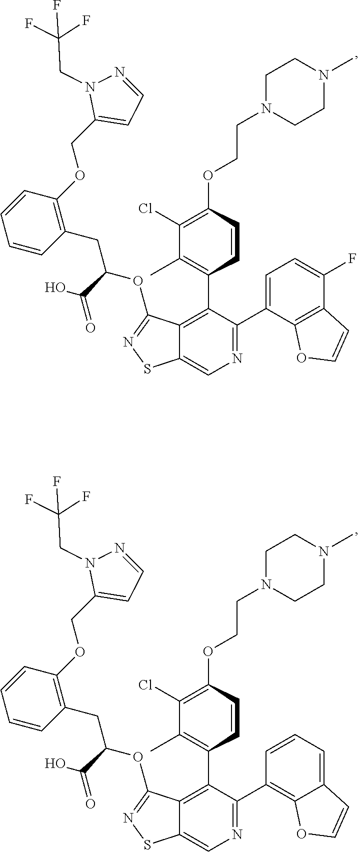

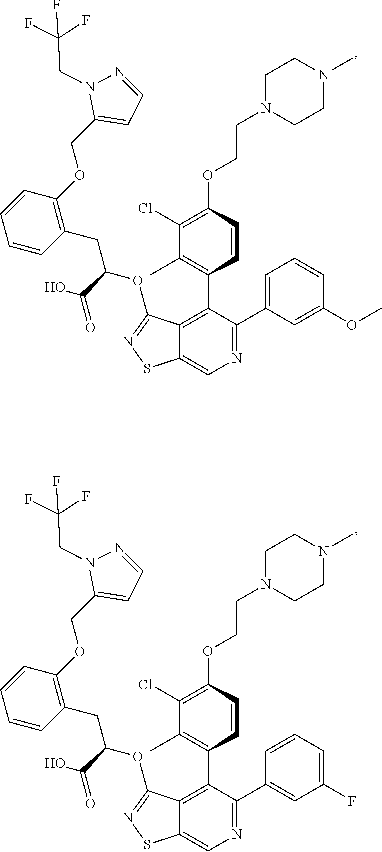

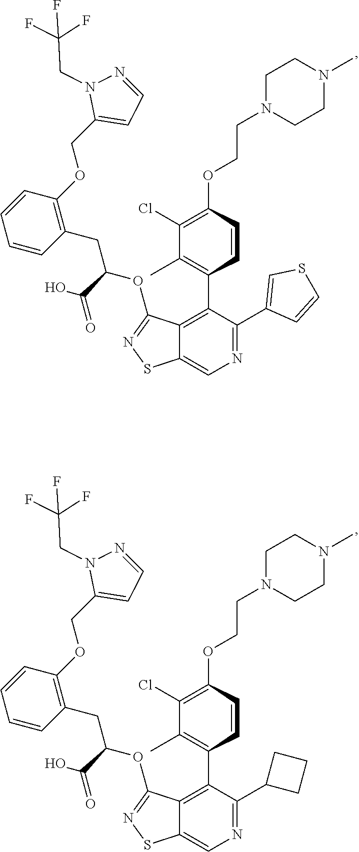

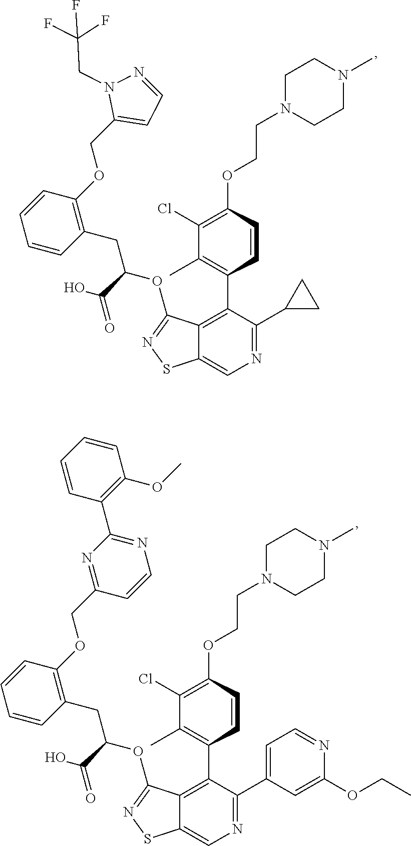

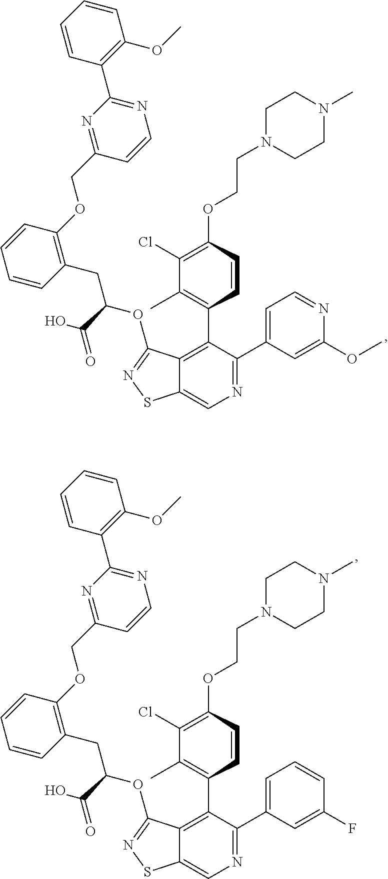

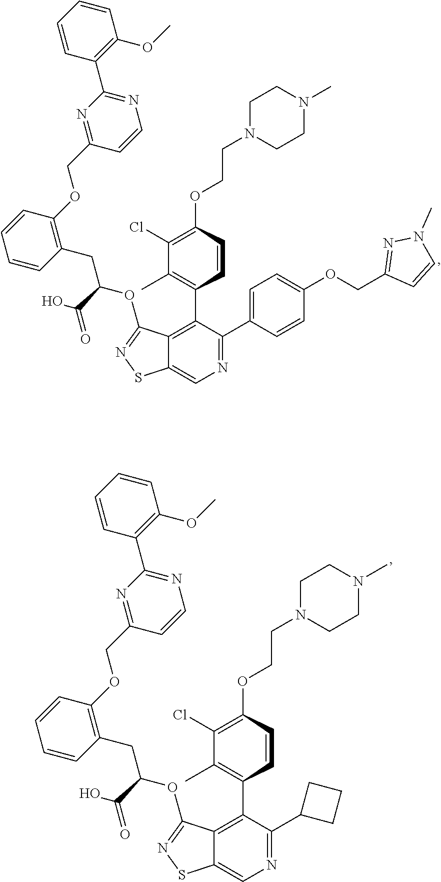

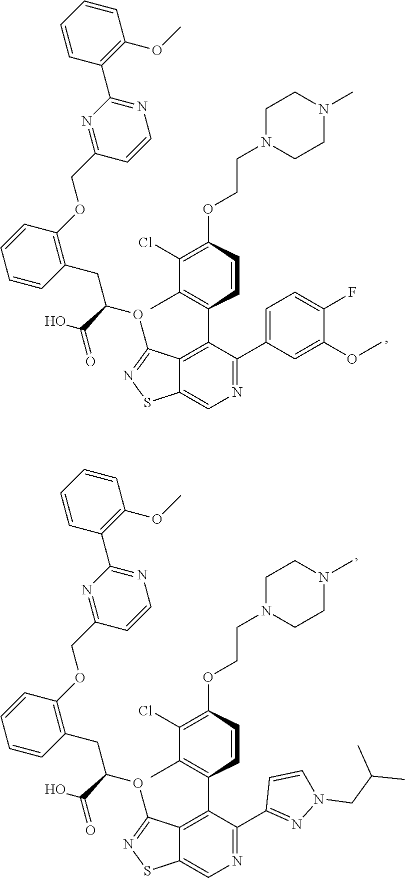

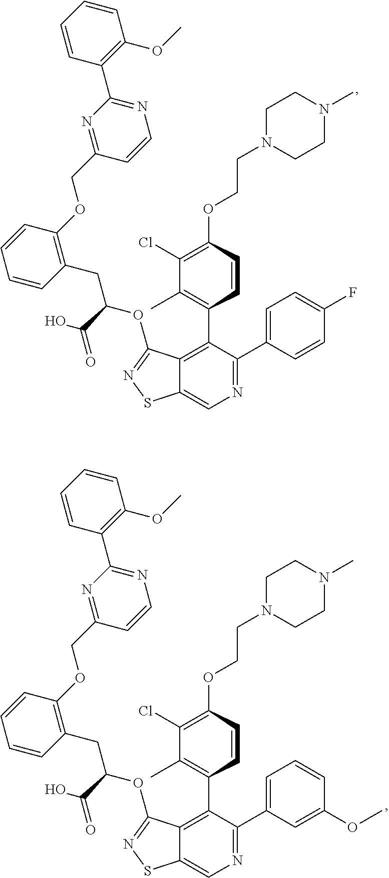

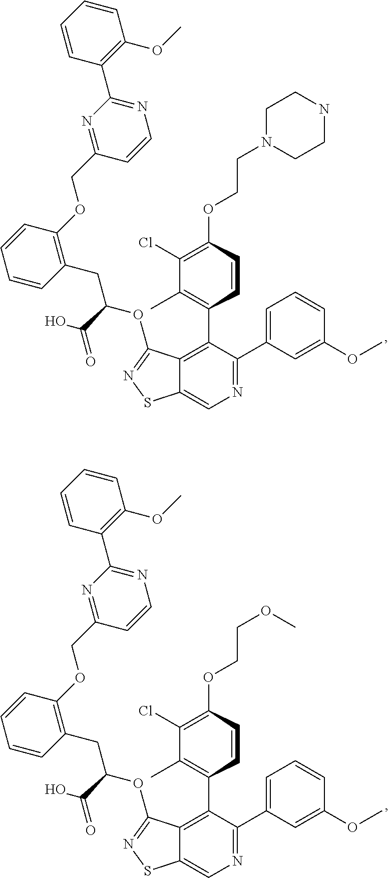

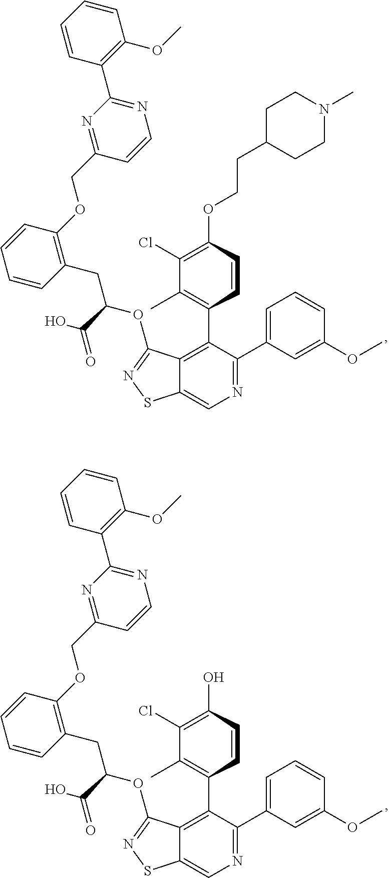

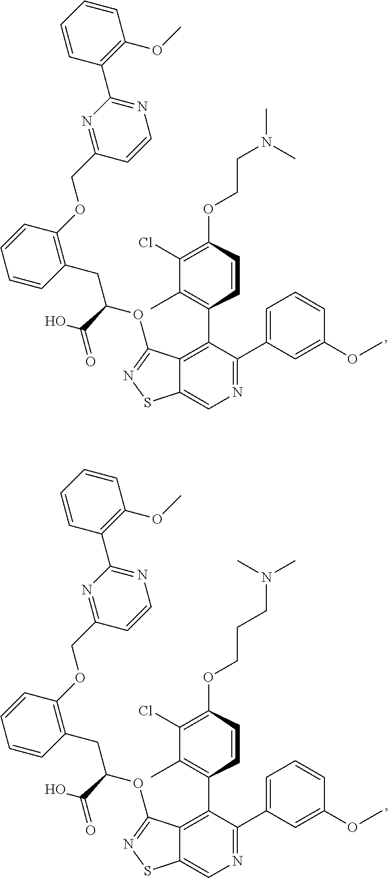

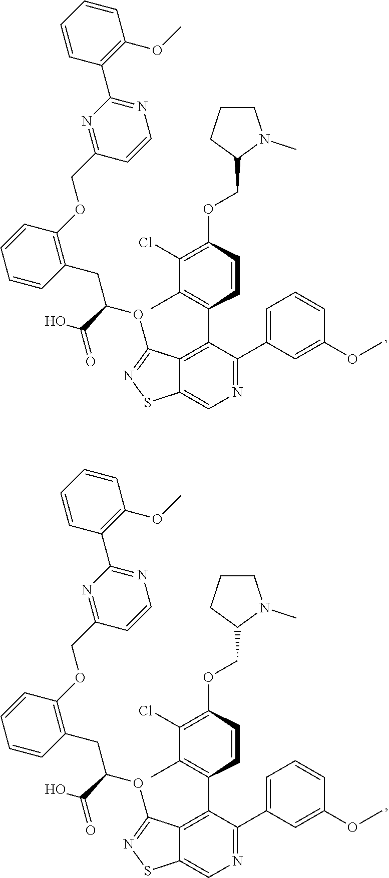

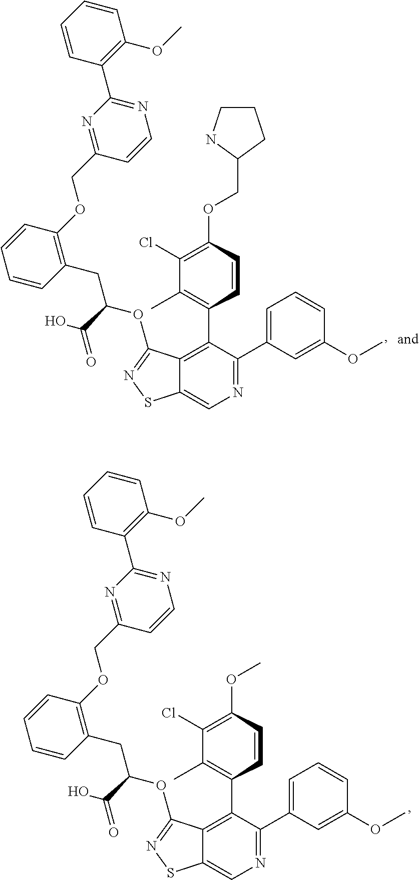

or a pharmaceutically acceptable salt thereof.

In some aspects, the invention relates to a compound of Formula III having a structure selected from:

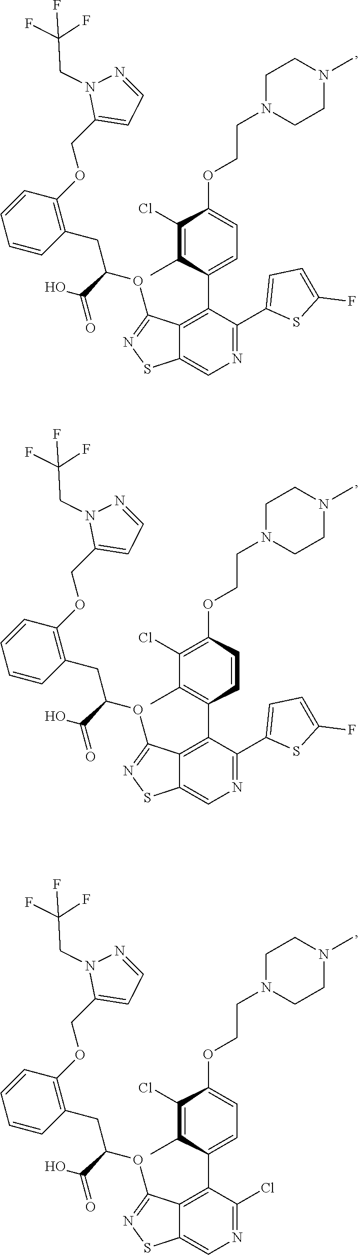

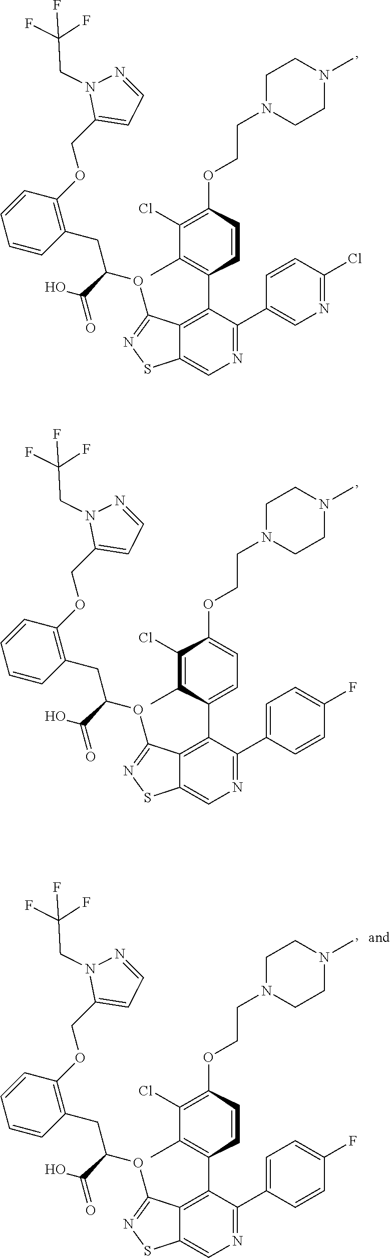

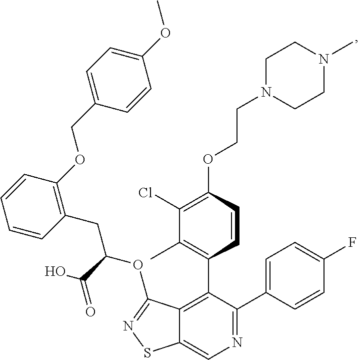

or a pharmaceutically acceptable salt thereof.

In particular aspects, the compound is selected from:

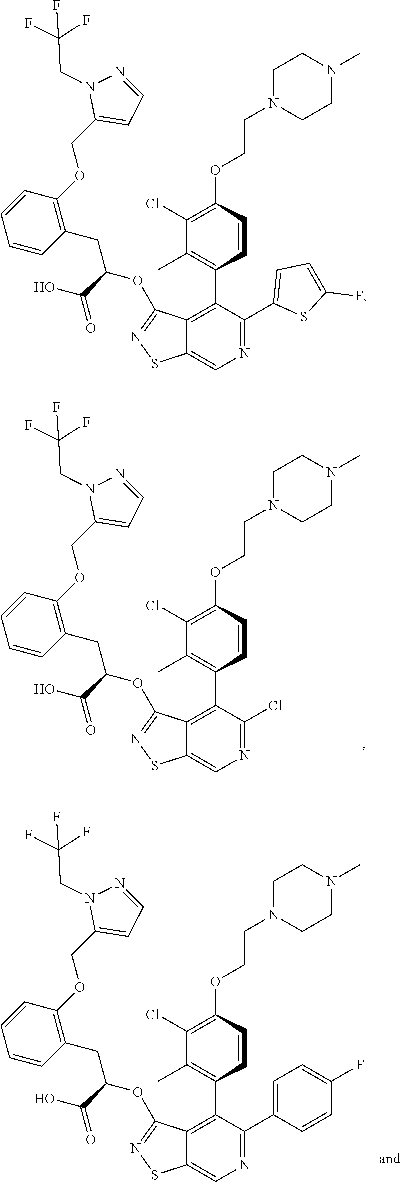

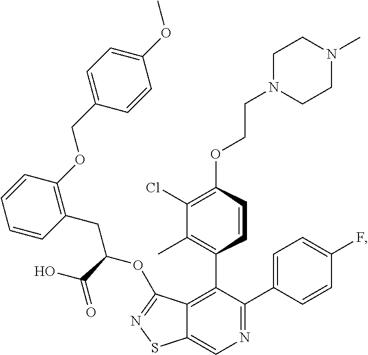

or a

pharmaceutically acceptable salt thereof.

In certain embodiments, the invention relates to a pharmaceutical composition comprising any of the compounds described herein and a pharmaceutically acceptable diluent or excipient.

Specific embodiments of the invention include those compounds listed in Table 1. The identifying number (“Cmpd”), the chemical structure (“Structure”), and the example method used to synthesize the compound (“Method”) are disclosed in Table 1 for each compound.

Specific embodiments of the invention include compounds of Formula IX, wherein E, R^{X3}, R^{X3a-1}, R^{X3a-2}, L³, Y_{b}, Z¹ and R^{Z1} are defined, in that order, as listed in each row of Table 4.

TABLE 1

| | | Example |
|---|---|---|
| Cmpd | Structure | Method |
| 1 | 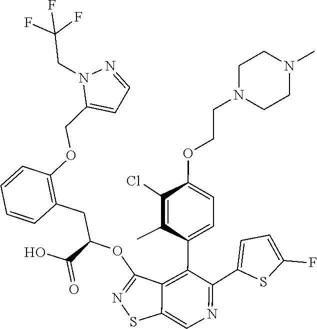 | 1 |
| 2 | 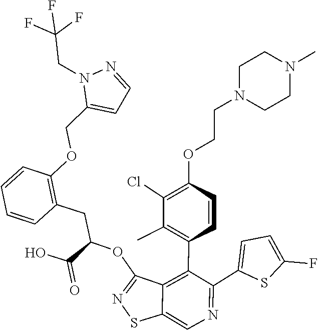 | 1 |
| 3 | 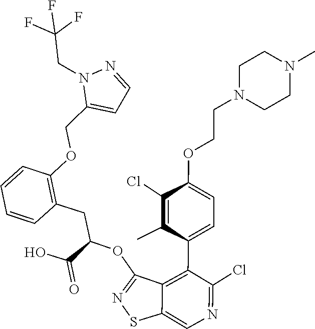 | 1 |
| 4 | 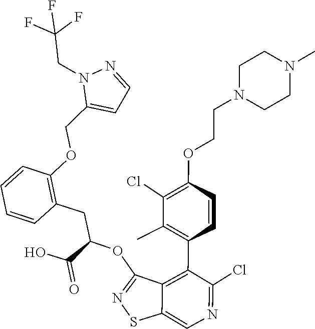 | 1 |
| 5 | 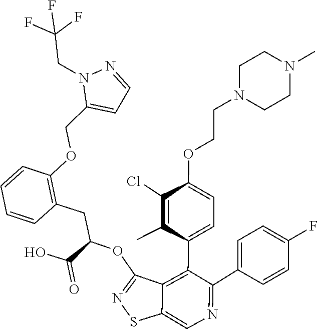 | 1 |
| 6 | 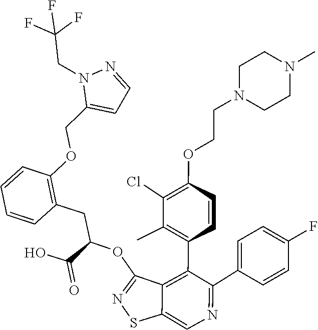 | 1 |
| 7 | 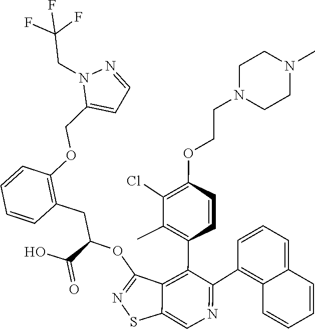 | 1 |
| 8 | 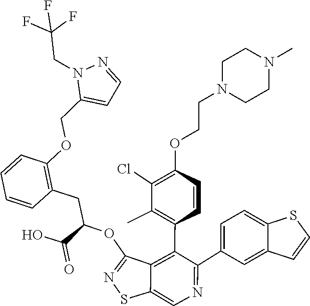 | 1 |
| 9 | 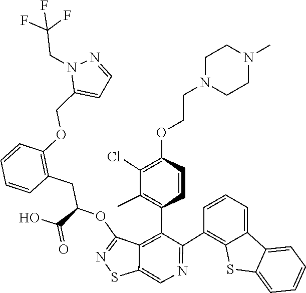 | 1 |
| 10 | 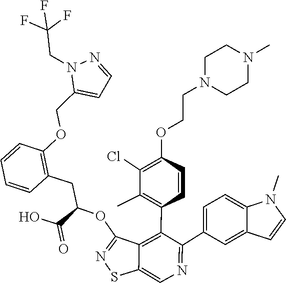 | 1 |
| 11 | 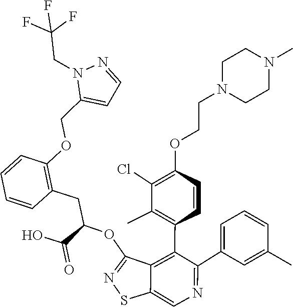 | 1 |
| 12 | 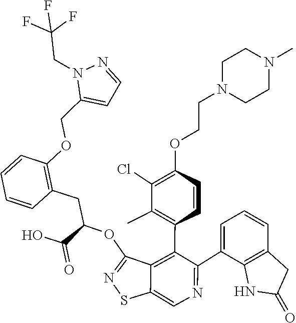 | 1 |
| 13 | 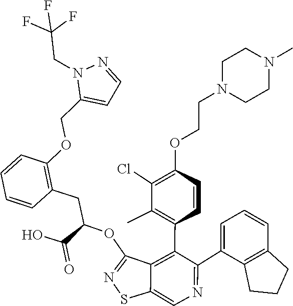 | 1 |
| 14 | 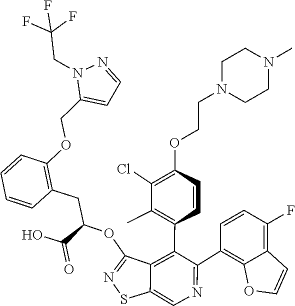 | 1 |
| 15 | 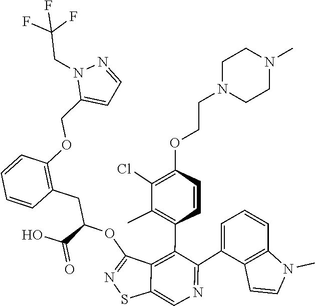 | 1 |
| 16 | 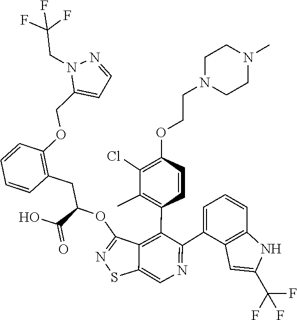 | 1 |
| 17 | 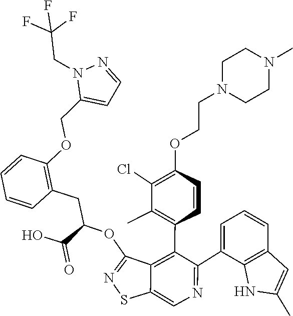 | 1 |
| 18 | 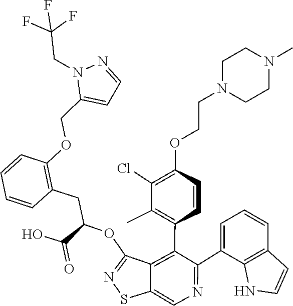 | 1 |
| 19 | 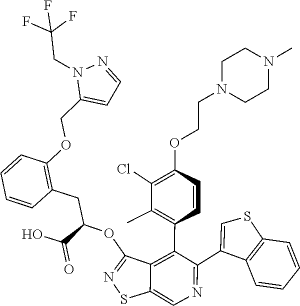 | 1 |
| 20 | 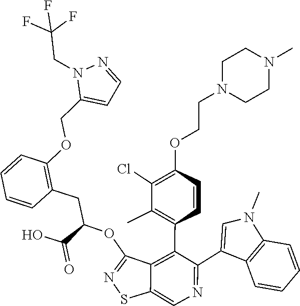 | 1 |
| 21 | 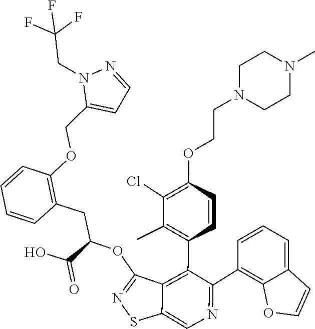 | 1 |
| 22 | 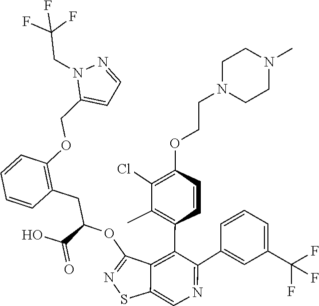 | 1 |
| 23 | 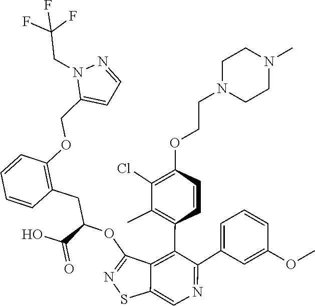 | 1 |
| 24 | 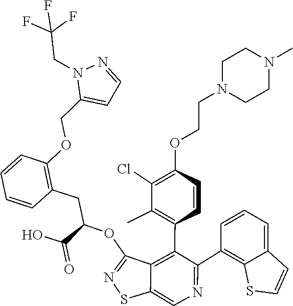 | 1 |
| 25 | 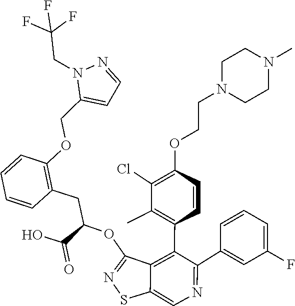 | 1 |
| 26 | 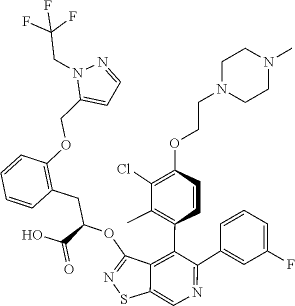 | 1 |
| 27 | 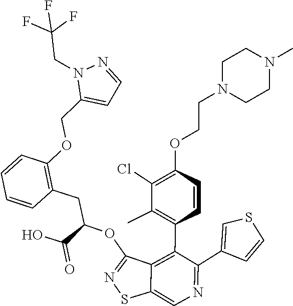 | 1 |
| 28 | 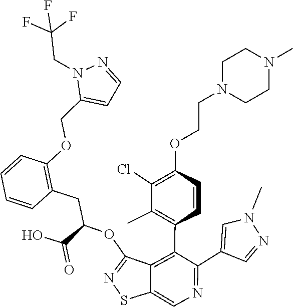 | 1 |
| 29 | 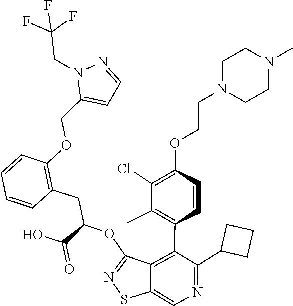 | 1 |
| 30 | 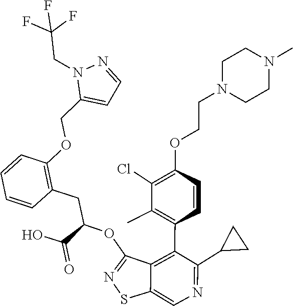 | 1 |
| 31 | 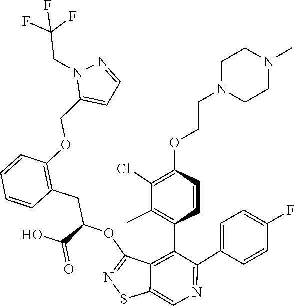 | 2 |
| 32 | 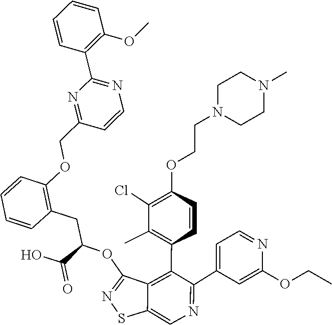 | 3 |
| 33 | 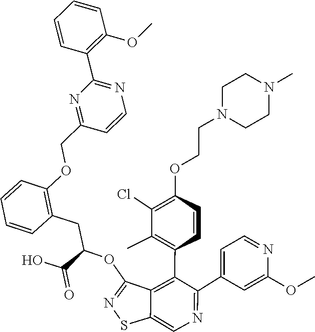 | 3 |
| 34 | 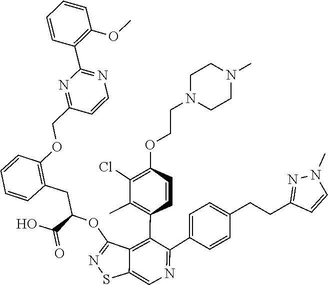 | 3 |
| 35 | 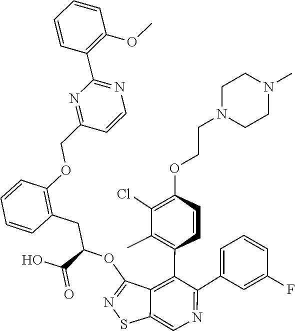 | 3 |
| 36 | 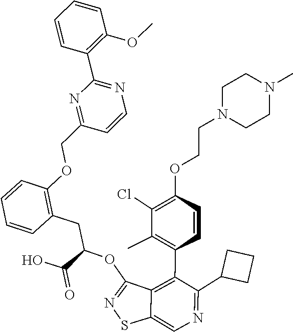 | 3 |
| 37 | 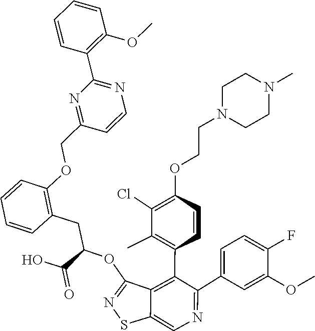 | 3 |
| 38 | 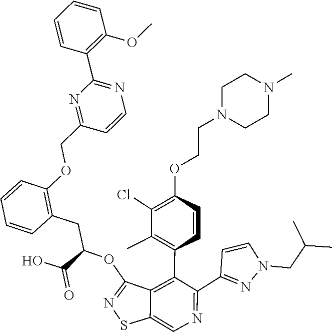 | 3 |
| 39 | 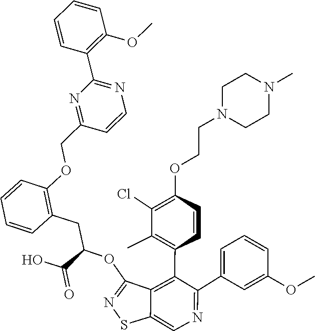 | 3 |
| 40 | 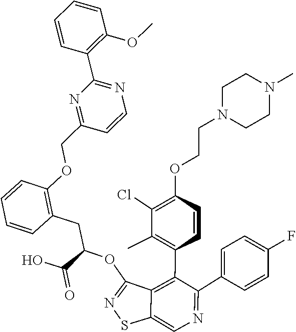 | 3 |
| 41 | 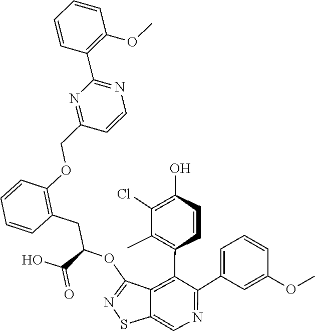 | 4 |
| 42 | 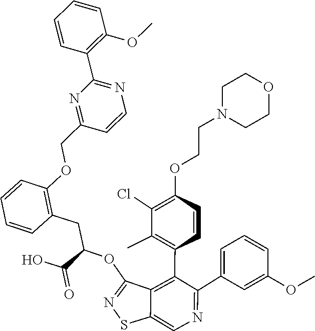 | 4 |
| 43 | 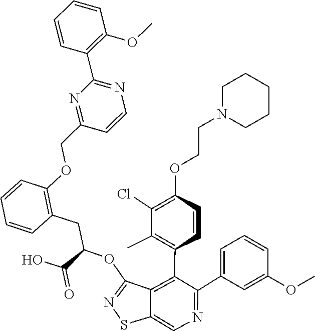 | 4 |
| 44 | 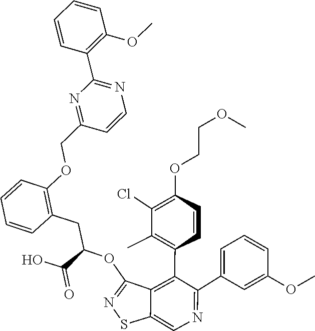 | 4 |
| 45 | 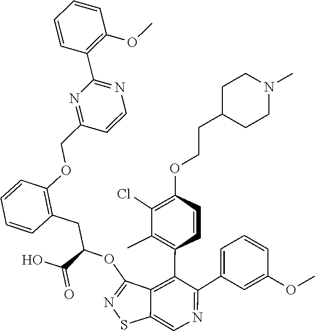 | 4 |
| 46 | 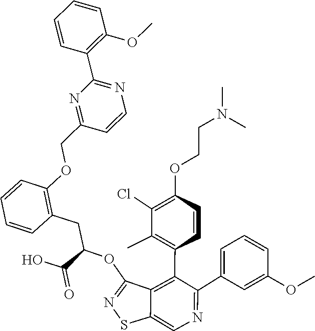 | 4 |
| 47 | 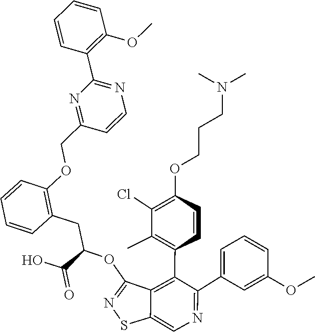 | 4 |
| 48 | 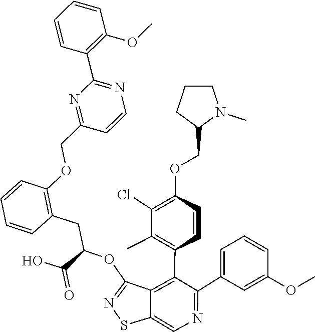 | 4 |
| 49 | 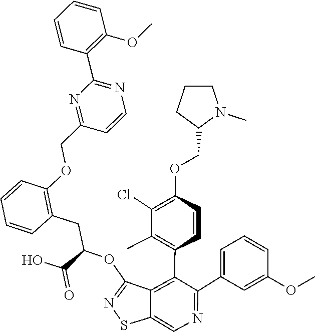 | 4 |
| 50 | 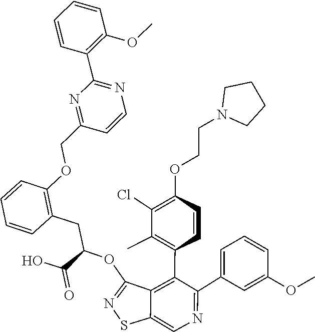 | 4 |
| 51 | 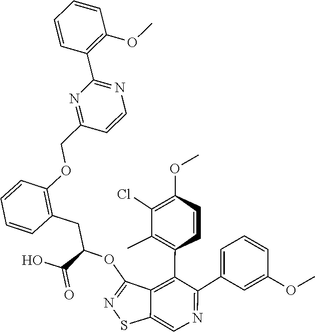 | 4 |
| 52 | 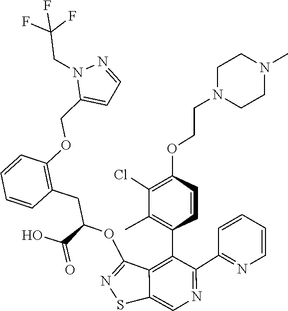 | 1 |
| 53 | 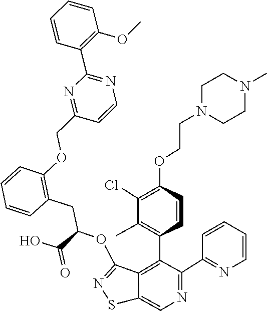 | 3 |
| 54 | 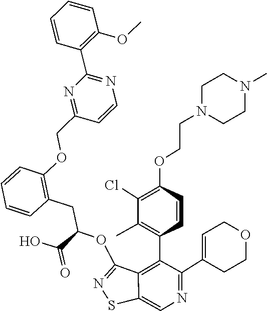 | 3 |
| 55 | 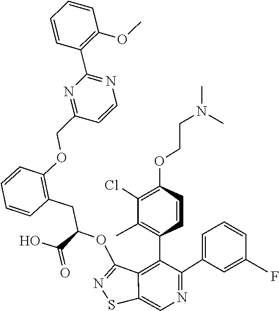 | 5 |
| 56 | 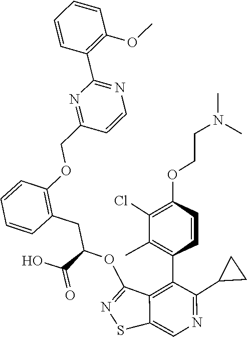 | 5 |
| 57 | 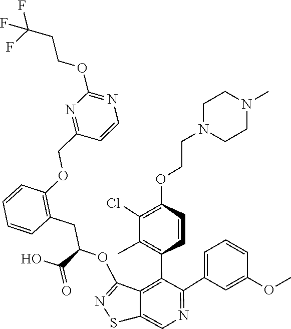 | 6 |
| 58 | 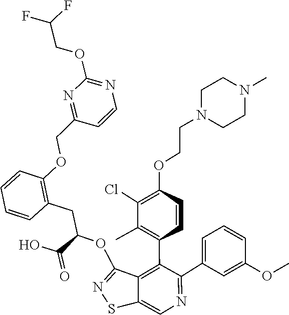 | 6 |
| 59 | 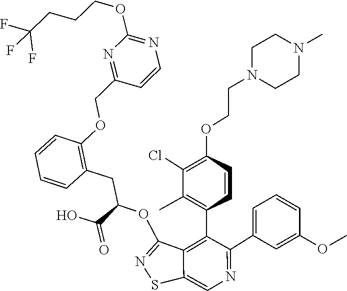 | 6 |
| 60 | 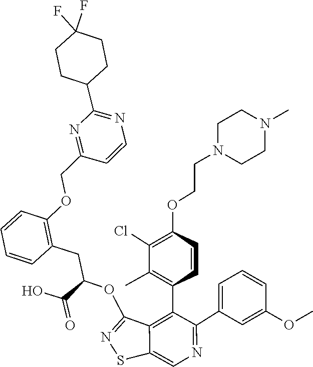 | 6 |
| 61 | 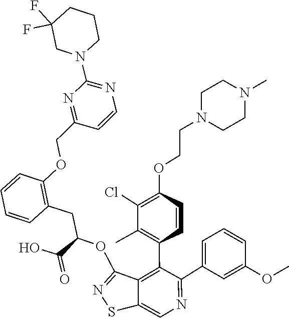 | 6 |
| 62 | 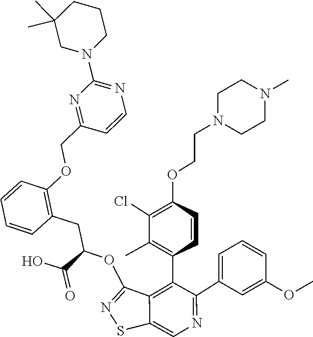 | 6 |
| 63 | 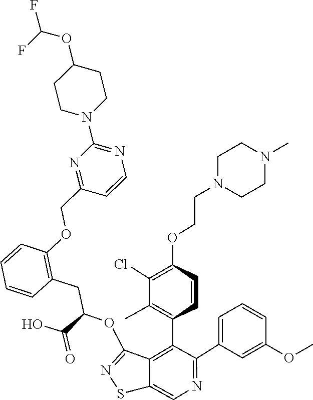 | 6 |
| 64 | 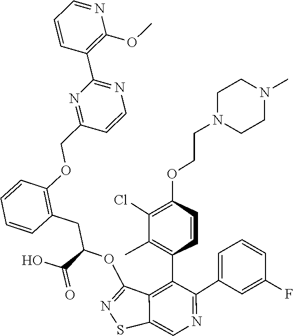 | 7 |
| 65 | 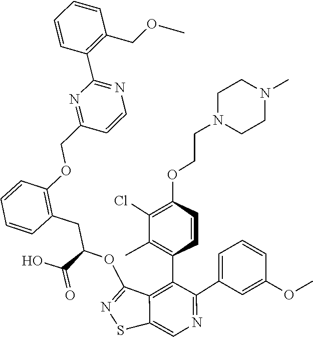 | 7 |
| 66 | 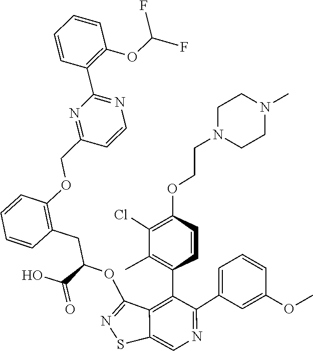 | 7 |
| 67 | 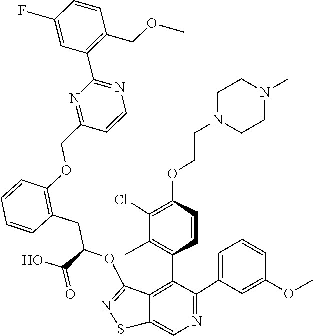 | 7 |
| 68 | 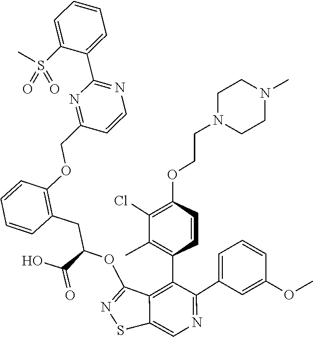 | 7 |
| 69 | 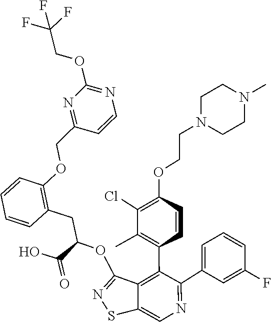 | 8 |
| 70 | 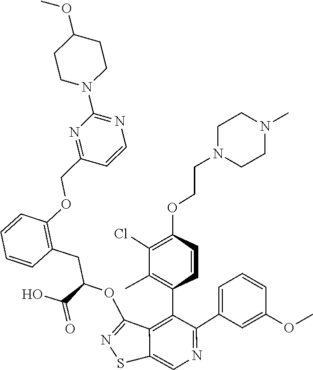 | 8 |
| 71 | 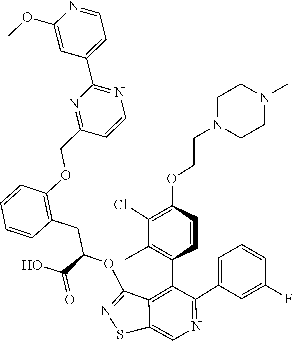 | 7 |
| 72 | 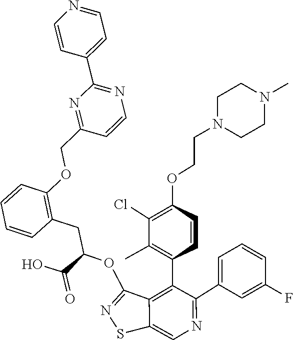 | 7 |
| 73 | 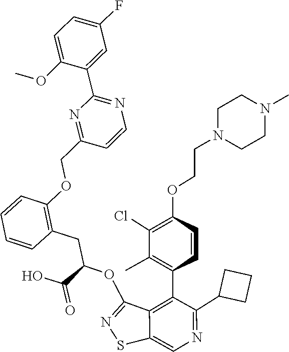 | 7 |
| 74 | 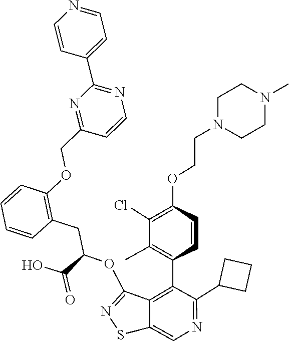 | 7 |
| 75 | 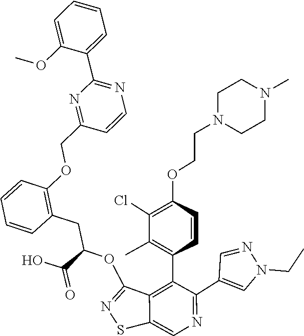 | 3 |
| 76 | 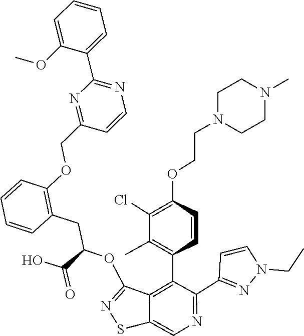 | 3 |
| 77 | 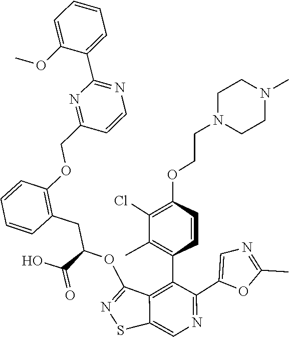 | 3 |
| 78 | 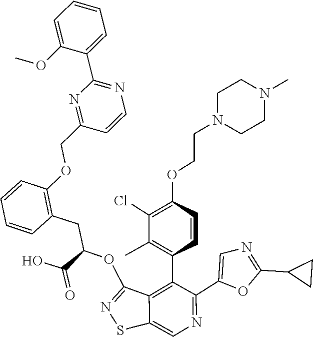 | 3 |
| 79 | 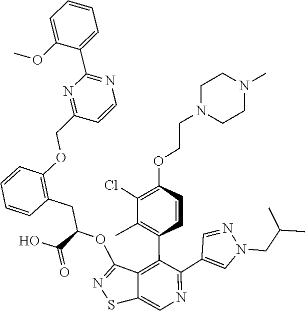 | 3 |
| 80 | 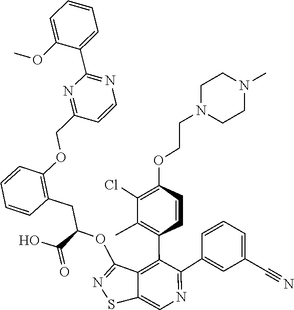 | 3 |
| 81 | 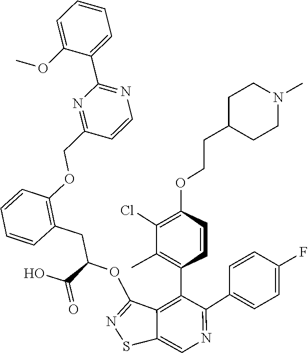 | 4 |
| 82 | 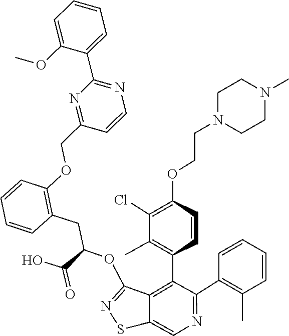 | 3 |
| 83 | 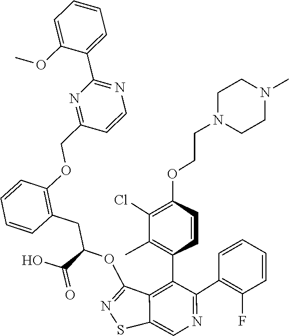 | 3 |
| 84 | 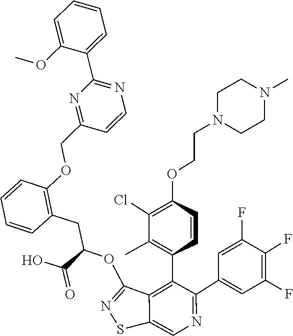 | 3 |
| 85 | 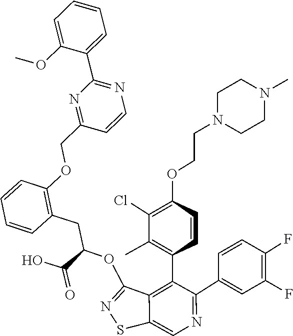 | 3 |
| 86 | 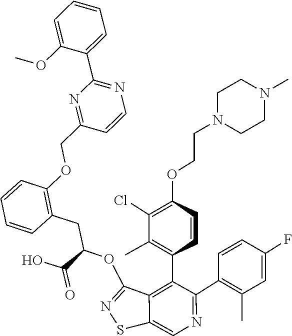 | 3 |
| 87 | 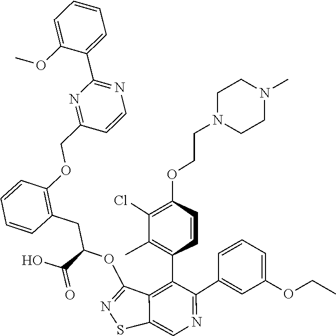 | 3 |
| 88 | 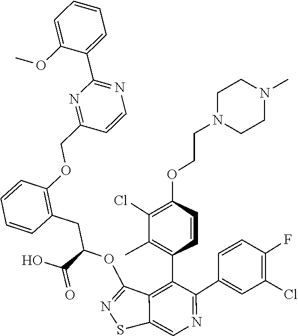 | 3 |
| 89 | 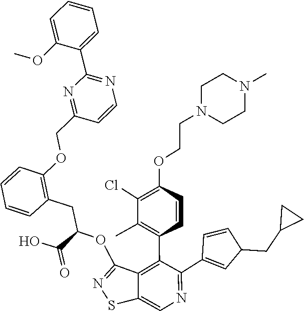 | 3 |
| 90 | 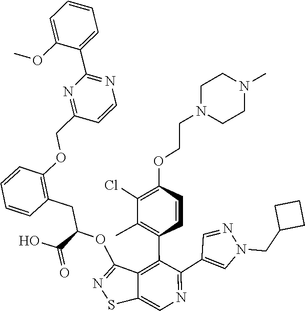 | 3 |
| 91 | 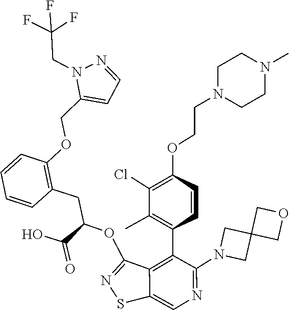 | 1 |
| 92 | 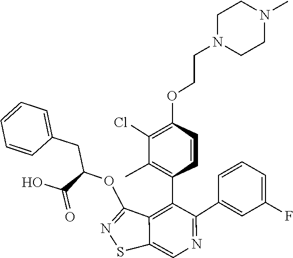 | 9 |
| 93 | 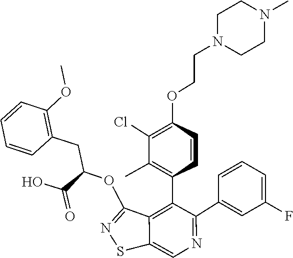 | 9 |
| 94 | 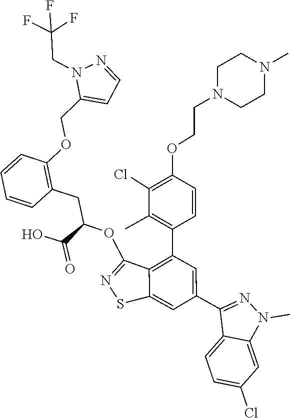 [SMILES: Cc1c(-c2cc(-c3nn(C)c4cc(Cl)ccc34)cc3snc(O[C@H](Cc4ccccc4OCc4ccnn4CC(F)(F)F)C(=O)O)c23)ccc(OCCN2CCN(C)CC2)c1Cl] | 10 |
| 95 | 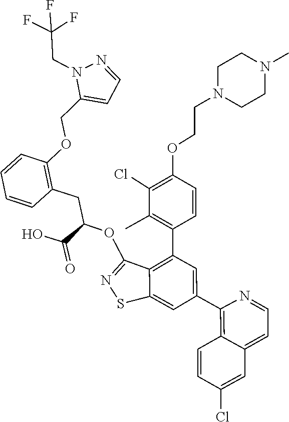 [SMILES: Cc1c(-c2cc(-c3nccc4cc(Cl)ccc34)cc3snc(O[C@H](Cc4ccccc4OCc4ccnn4CC(F)(F)F)C(=O)O)c23)ccc(OCCN2CCN(C)CC2)c1Cl] | 10 |
| 96 | 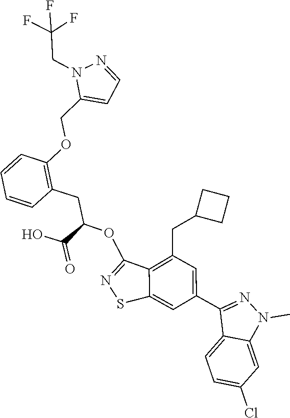 [SMILES: Cn1nc(-c2cc(CC3CCC3)c3c(O[C@H](Cc4ccccc4OCc4ccnn4CC(F)(F)F)C(=O)O)nsc3c2)c2ccc(Cl)cc21] | 10 |
| 97 | 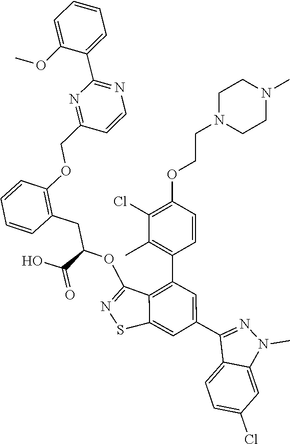 [SMILES: COc1ccccc1-c1nccc(COc2ccccc2C[C@@H](Oc2nsc3cc(-c4nn(C)c5cc(Cl)ccc45)cc(-c4ccc(OCCN5CCN(C)CC5)c(Cl)c4C)c23)C(=O)O)n1] | 10 |
| 98 | 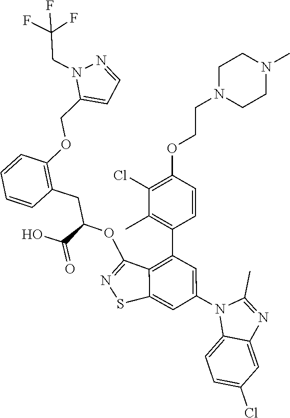 [SMILES: Cc1c(-c2cc(-n3c(C)nc4cc(Cl)ccc43)cc3snc(O[C@H](Cc4ccccc4OCc4ccnn4CC(F)(F)F)C(=O)O)c23)ccc(OCCN2CCN(C)CC2)c1Cl] | 10 |
| 99 | 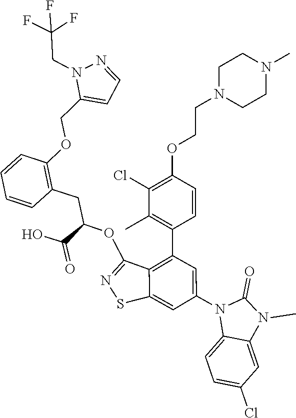 [SMILES: Cc1c(-c2cc(-n3c(=O)n(C)c4cc(Cl)ccc43)cc3snc(O[C@H](Cc4ccccc4OCc4ccnn4CC(F)(F)F)C(=O)O)c23)ccc(OCCN2CCN(C)CC2)c1Cl] | 10 |
| 100 | 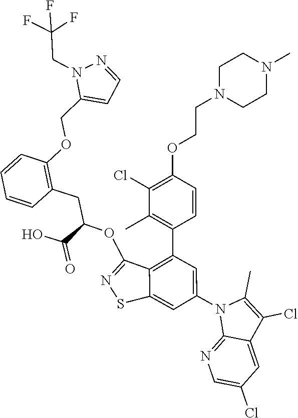 [SMILES: Cc1c(-c2cc(-n3c(C)c(Cl)c4cc(Cl)cnc43)cc3snc(O[C@H](Cc4ccccc4OCc4ccnn4CC(F)(F)F)C(=O)O)c23)ccc(OCCN2CCN(C)CC2)c1Cl] | 10 |
| 101 | 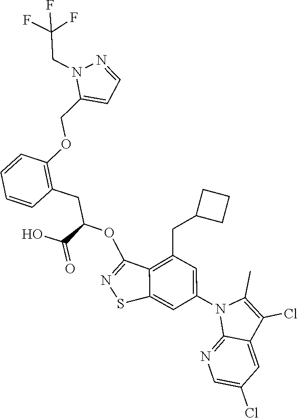 [SMILES: Cc1c(Cl)c2cc(Cl)cnc2n1-c1cc(CC2CCC2)c2c(O[C@H](Cc3ccccc3OCc3ccnn3CC(F)(F)F)C(=O)O)nsc2c1] | 10 |
| 102 | 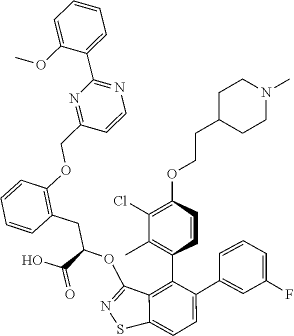 | 4 |
| 103 | 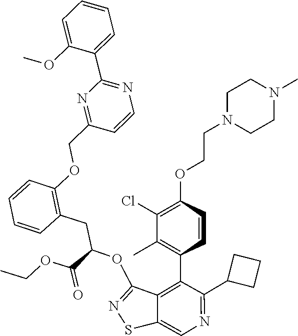 | 3 |
| 104 | 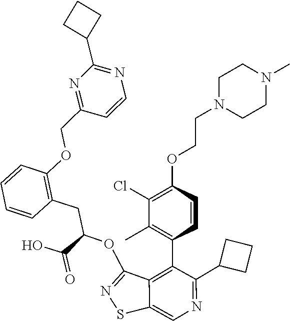 | 6 |
| 105 | 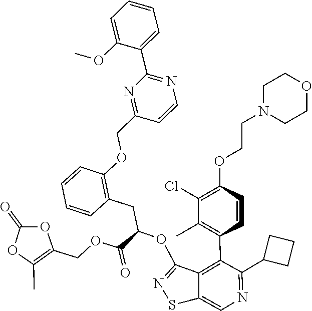 | 4 |
| 106 | 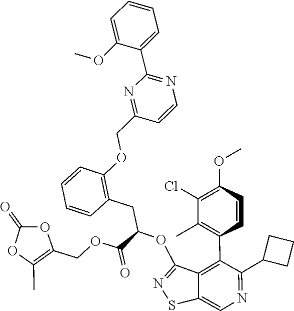 | 4 |
| 107 | 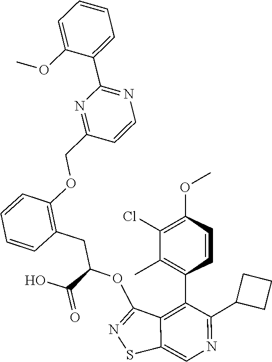 | 4 |
| 108 | 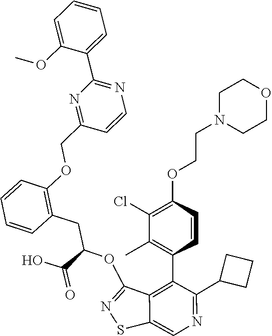 | 4 |
| 109 | 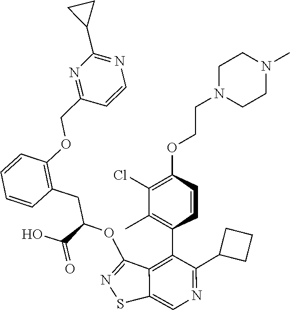 | 11 |
| 110 | 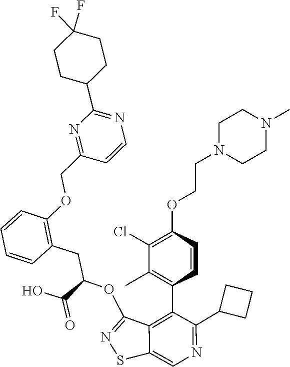 | 12 |
| 111 | 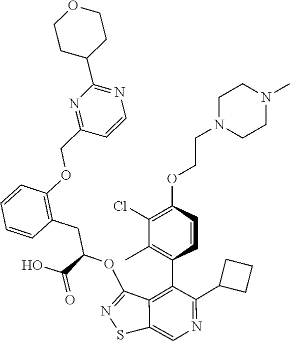 | 12 |
| 112 | 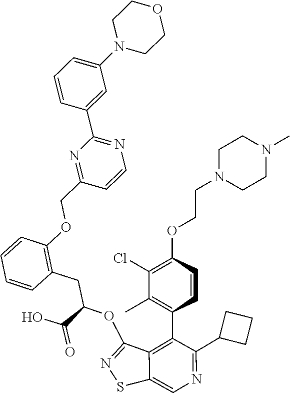 | 7 |
| 113 | 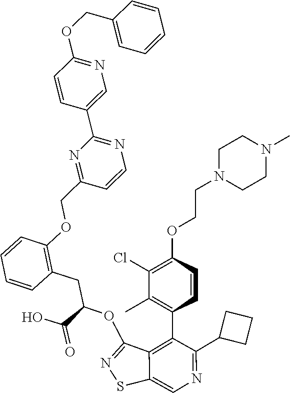 | 7 |
| 114 | 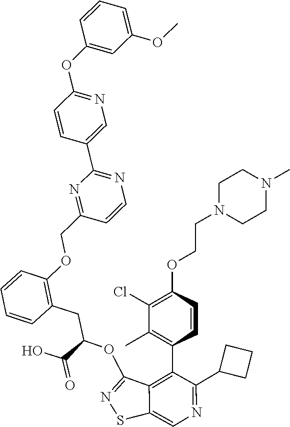 | 7 |
| 115 | 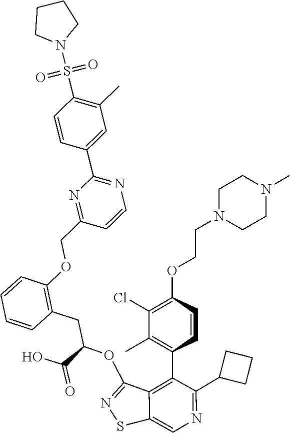 | 7 |
| 116 | 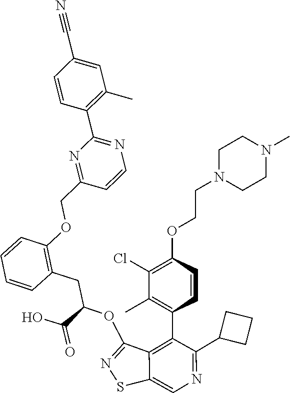 | 7 |
| 117 | 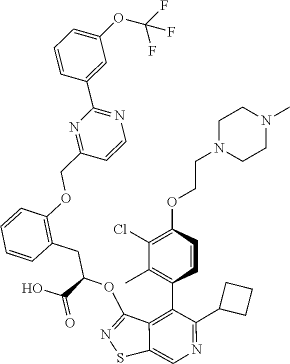 | 11 |
| 118 | 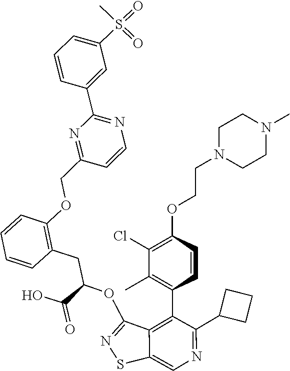 | 11 |
| 119 | 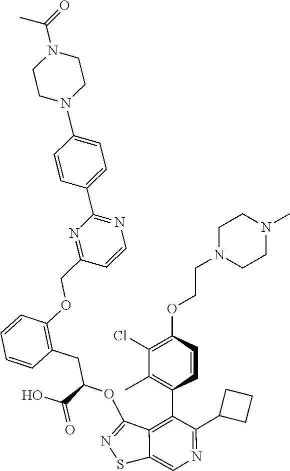 | 7 |
| 120 | 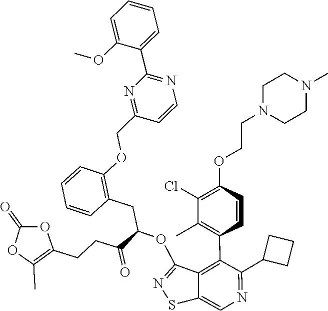 | 4 |
| 121 | 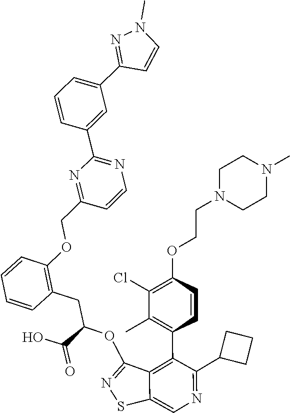 | 7 |
| 122 | 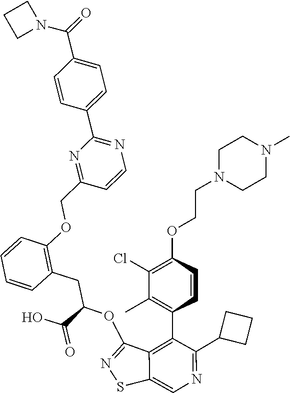 | 7 |
| 123 | 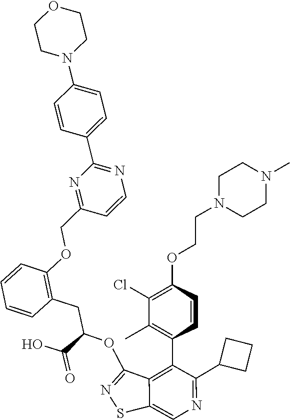 | 11 |
| 124 | 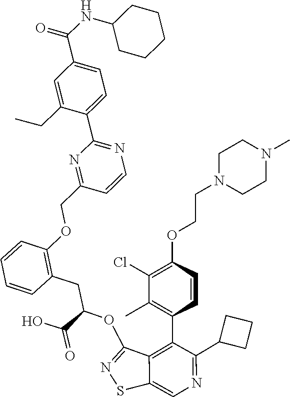 | 7 |
| 125 | 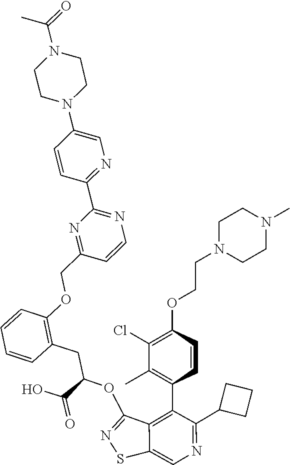 | 7 |
| 126 | 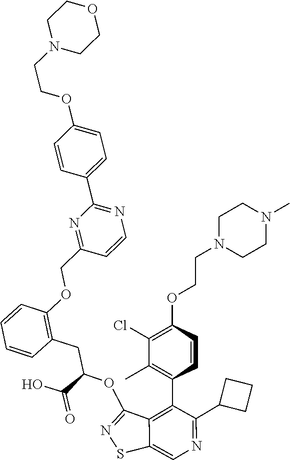 | 7 |

### Example Methods of Treatment/Use

The compounds described herein are inhibitors of MCL1 and therefore may be useful for treating diseases wherein the underlying pathology is (at least in part) mediated by MCL1 or the dysregulation of its normal activity. Such diseases include cancer and other diseases in which there is a disorder of cell proliferation, apoptosis, or differentiation.

In certain embodiments, the method of treating cancer in a subject in need thereof comprises administering to the subject an effective amount of any of the compounds described herein, or a pharmaceutically acceptable salt thereof. For example, the cancer may be selected from carcinoma (e.g., a carcinoma of the endometrium, bladder, breast, or colon (e.g., colorectal carcinomas such as colon adenocarcinoma and colon adenoma)), sarcoma (e.g., a sarcoma such as Kaposi's, osteosarcoma, tumor of mesenchymal origin, for example fibrosarcoma or habdomyosarcoma), kidney, epidermis, liver, lung (e.g., adenocarcinoma, small cell lung cancer and non-small cell lung carcinomas), esophagus, gall bladder, ovary, pancreas (e.g., exocrine pancreatic carcinoma), stomach, cervix, thyroid, nose, head and neck, prostate, and skin (e.g., squamous cell carcinoma), human breast cancers (e.g., primary breast tumors, node-negative breast cancer, invasive duct adenocarcinomas of the breast, non-endometrioid breast cancers), familial melanoma, and melanoma. Other examples of cancers that may be treated with a compound of the invention include hematopoietic tumors of lymphoid lineage (e.g. leukemia, acute lymphocytic leukemia, mantle cell lymphoma, chronic lymphocytic leukemia, B-cell lymphoma (such as diffuse large B cell lymphoma), T-cell lymphoma, multiple myeloma, Hodgkin's lymphoma, non-Hodgkin's lymphoma, hairy cell lymphoma, and Burkett's lymphoma), and hematopoietic tumors of myeloid lineage, for example acute and chronic myelogenous leukemias, myelodysplastic syndrome, and promyelocytic leukemia. Other cancers include a tumor of the central or peripheral nervous system, for example astrocytoma, neuroblastoma, glioma or schwannoma; seminoma; teratocarcinoma; xeroderma pigmentosum; retinoblastoma; keratoctanthoma; and thyroid follicular cancer.

In particular embodiments, the cancer is selected from head and neck cancer, sarcoma, melanoma, myeloma, lymphoma, lung cancer (including non-small cell lung cancer and small cell lung cancer), breast cancer, pancreatic cancer, thyroid cancer, colorectal cancer, ovarian cancer and acute myelogenous leukemia.

In some aspects, the subject is a mammal, for example, a human.

Further disclosed herein are methods of inhibiting MCL1 in a cell comprising contacting said cell with any of the compounds described herein, or a pharmaceutically acceptable salt thereof, such that the function of MCL1 is inhibited in said cell. For example, the cell is a cancer cell. In preferred embodiments, proliferation of the cell is inhibited or cell death is induced.

Further disclosed herein is a method of treating a disease treatable by inhibition of MCL1 in a subject, comprising administering to the subject in recognized need of such treatment, an effective amount of any of the compounds described herein and/or a pharmaceutically acceptable salt thereof. Diseases treatable by inhibition of MCL1 include, for example, diseases characterized by dysregulation of apoptosis, including hyperproliferative diseases such as cancer. Further exemplary diseases include head and neck cancer, sarcoma, melanoma, myeloma, lymphoma, lung cancer (including non-small cell lung cancer and small cell lung cancer), breast cancer, pancreatic cancer, thyroid cancer, colorectal cancer, ovarian cancer and acute myelogenous leukemia.

The methods of treatment comprise administering a compound of the invention, or a pharmaceutically acceptable salt thereof, to a subject in need thereof. Individual embodiments include methods of treating any one of the above-mentioned disorders or diseases by administering an effective amount of a compound of the invention, or a pharmaceutically acceptable salt thereof, to a subject in need thereof.

Certain embodiments include a method of modulating MCL1 activity in a subject comprising administering to the subject a compound of the invention, or a pharmaceutically acceptable salt thereof. Additional embodiments provide a method for the treatment of a disorder or a disease mediated by MCL1 in a subject in need thereof, comprising administering to the subject an effective amount of the compound of Formula I, II, III, IV, V, VI, VII, VIII, IX or X, or a pharmaceutically acceptable salt thereof. Other embodiments of the invention provide a method of treating a disorder or a disease mediated by MCL1, in a subject in need of treatment thereof comprising administering an effective amount of a compound of the invention, or a pharmaceutically acceptable salt thereof, wherein the disorder or the disease is selected from carcinomas with genetic aberrations that activate MCL1 activity. These include, but are not limited to, cancers.

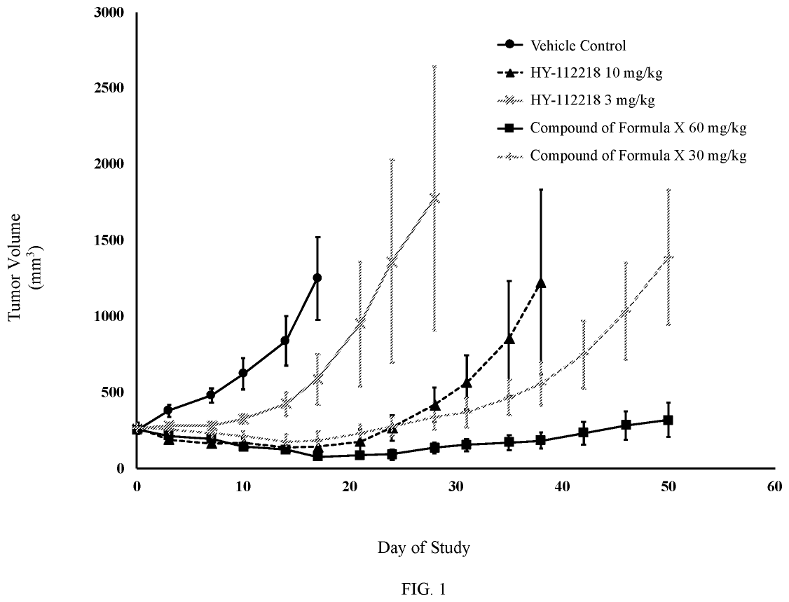

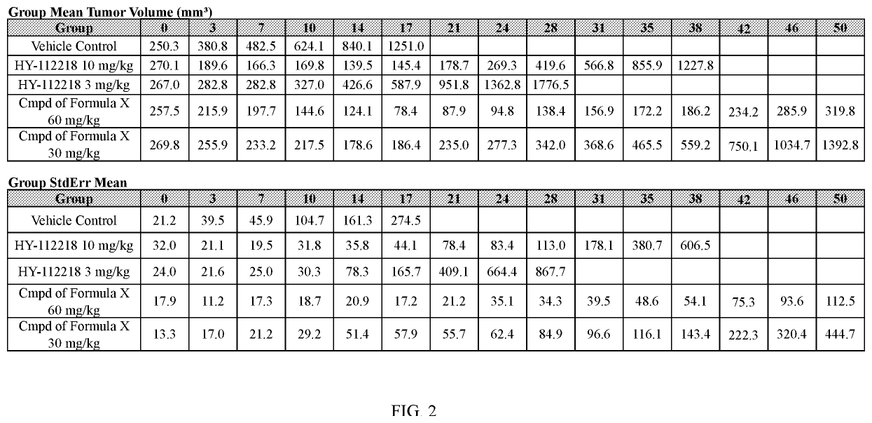

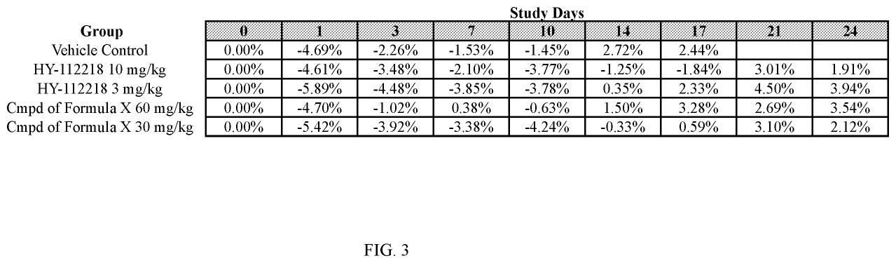

FIG. 1 are the results of an AMO-1 myeloma cell line xenograft study with a compound of Formula X and with an MCL1 inhibitor being tested in humans and available as catalog number HY-112218 on Oct. 2, 2020 (MedChemExpress LLC, New Jersey, USA). Eight mice were used per group, and mice were dosed by intravenous injection (IV) daily (QD) for the first 5 days of the study with various concentrations of the compound of Formula X or with HY-112218. The 10 mg/kg dose of HY-112218 and the 60 mg/kg dose of the compound of Formula X are about the maximum tolerated dose for mice and represent a theoretical efficacious dose for humans. These results demonstrate an unexpected prolonged tumor growth inhibition with the compound of Formula X, whereas tumors treated with HY-112218 resumed growth at or about Day 21 of the study. FIG. 2 shows the change in tumor volume results of the AMO-1 myeloma cell line xenograft study over time, and FIG. 3 is the tabulation of the percentage weight changes per day of mice in the AMO-1 myeloma cell line xenograft study.

The present method also provides the use of a compound of invention, or a pharmaceutically acceptable salt thereof, for the treatment of a disorder or disease mediated by MCL1.

In some embodiments, a compound of the invention, or a pharmaceutically acceptable salt thereof, is used for the treatment of a disorder or a disease mediated by MCL1.

Yet other embodiments of the present method provide a compound according to Formula I, II, III, IV, V, VI, VII, VIII, IX or X, or a pharmaceutically acceptable salt thereof, for use as a medicament.

Still other embodiments of the present method encompass the use of a compound of Formula I, II, III, IV, V, VI, VII, VIII, IX or X, or a pharmaceutically acceptable salt thereof, in the manufacture of a medicament for the treatment of a disorder or disease mediated by MCL1.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### Exemplification

Synthetic Protocols

Compounds as disclosed herein can be synthesized via a number of specific methods. The examples which outline specific synthetic routes, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### Example A: Synthesis of Compounds A2 Through A9

### Synthesis of Compound A2

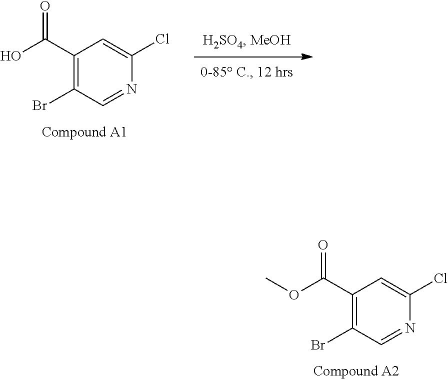

To a mixture of compound A1 (140 g, 592.1 mmol, 1.00 eq) in MeOH (554.3 g, 17.3 mol, 700 mL, 29.2 eq) was added H₂SO₄ (116.1 g, 1.18 mol, 63.1 mL, 2.00 eq) at 0° C. The mixture was stirred at 85° C. for 12 hours. LCMS (product Rt=0.867 min, m/z=252.4 (M+1)⁺) showed compound A1 was consumed and a main peak with desired MS was formed. The mixture was concentrated under vacuum to give residue which was adjusted to pH 8 by aqueous NaHCO₃. The mixture was extracted with ethyl acetate (1.00 L, 2 times). The combined organic phase was washed with brine (500 mL, 2 times), dried with Na₂SO₄, filtered and concentrated under reduced pressure to yield compound A2 (106.0 g, 415.15 mmol, 70.1% yield, 98.1% purity) as a yellow oil, and was confirmed by LCMS (compound A2 Rt=0.866 min, m/z=252.4 (M+1)⁺) and HNMR. Compound A2 in its yellow oil state was used directly for the next step.

¹H NMR (400 MHz, CDCl₃):

### δ 8.60 (s, 1H), 7.65 (s, 1H), 3.95 (s, 3H) ppm.

### Synthesis of Compound A3

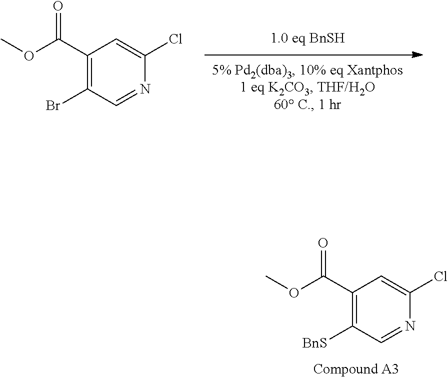

To a mixture of compound A2 (106.0 g, 423.2 mmol, 1.00 eq), Pd₂(dba)₃ (19.38 g, 21.16 mmol, 0.05 eq), K₂CO₃ (58.5 g, 423.2 mmol, 1.00 eq), and Xantphos (24.5 g, 42.3 mmol, 0.10 eq) in THF/H₂O (530 mL/130 mL) was added drop-wise a solution of BnSH (52.6 g, 423.5 mmol, 49.6 mL, 1.00 eq) in THE (130 mL) at 60° C. under nitrogen atmosphere, and the mixture was stirred at 60° C. for 1 hour. LCMS (product Rt=1.006 min, m/z=294.5 (M+1)⁺) showed compound A2 was consumed and a main peak with desired MS was formed. The mixture was diluted with brine (300 mL), extracted with ethyl acetate (300 mL, 2 times), and the organic phase was washed with brine (300 mL, 2 times), dried over sodium sulfate and concentrated under vacuum to give a crude product, which was triturated in a solution of ethyl acetate/petroleum ether (½, 350 mL) to give impure compound A3 (85.0 g, crude) as green solid, which was confirmed by LC/MS (compound A3 Rt=1.038 min, m/z=294.1 (M+1)⁺) and HNMR. Compound A3 was used without further purification.

¹H NMR (400 MHz, CDCl₃):

### δ 8.29 (s, 1H), 7.68 (s, 1H), 7.34-7.21 (m, 5H), 4.17 (s, 2H), 3.40 (s, 3H) ppm.

### Synthesis of Compound A4

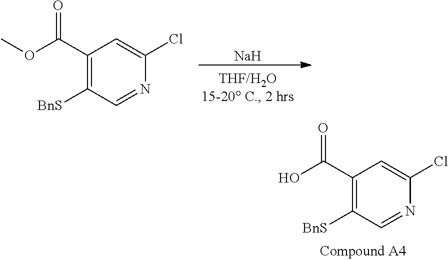

To a mixture of compound A3 (85.0 g, 289.3 mmol, 1.00 eq) in THF (160 mL) and H₂O (160 mL) was added NaOH (4 M, 144.67 mL, 2.00 eq) at 15-20° C. and the mixture was stirred at 15-20° C. for 2 hours. LCMS (compound A4 Rt=0.898 min, m/z=280.5 (M+1)⁺) showed compound A3 was consumed and the desired MS was found. The mixture was diluted with ethyl acetate (300.0 mL), adjusted to pH-5 with aqueous 6 M HCl. The formed solid was filtered and washed with water (20.0 mL) and dried under vacuum to give crude product, which was triturated in acetonitrile (250.0 mL) and filtered to give compound A4 (71.0 g, 252.3 mmol, 87.2% yield, 99.4% purity) as green solid and confirmed by LCMS (compound A4 Rt=0.898 min, m/z=280.0 (M+1)⁺) and HNMR. Compound A4 was used without further purification.

¹H NMR (400 MHz, DMSO-d₆):

### δ 8.51 (s, 1H), 7.74 (s, 1H), 7.28-7.43 (m, 5H), 4.38 (s, 2H) ppm.

### Synthesis of Compound A5

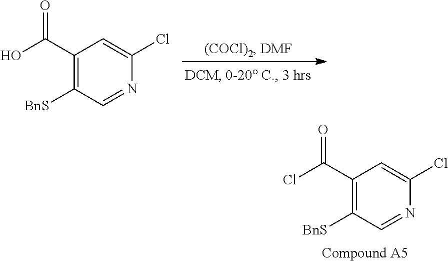

To a mixture of compound A4 (32.0 g, 114.4 mmol, 1.00 eq) and DMF (418.04 mg, 5.72 mmol, 440.0 μL, 0.05 eq) in DCM (350 mL) was added (COCI)₂ (29.0 g, 228.8 mmol, 20.0 mL, 2.00 eq) at 0° C., and the mixture was stirred at 20° C. for 3 hours. A sample was taken and quenched with a drop of lithium (tert-butoxycarbonyl) amide (prepared THE solution with n-BuLi at −78° C.) and LCMS (compound A5 Rt=0.996, compound A5 MS=379.1 (M+1)⁺) showed that most of compound A4 was consumed and a main peak with the desired MS was formed. The mixture was concentrated and co-evaporated with DCM (100 mL, 3 times) to give crude compound A5 (68.2 g, crude) as green gum. Compound A5 was used directly without further purification.

### Synthesis of Compound A6

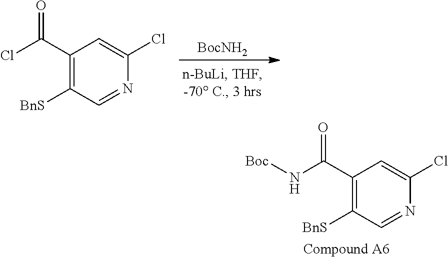

To a mixture of tert-butyl carbamate (20.0 g, 171.0 mmol, 1.50 eq) and TMEDA (19.9 g, 171.0 mmol, 25.8 mL, 1.50 eq) in THF (100.0 mL) was added n-BuLi (2.5 M, 68.4 mL, 1.50 eq) drop-wise at −70° C., and the formed mixture was stirred at −70° C. for 1 hour. Compound A5 (34.0 g, 114.02 mmol, 1.00 eq) was dissolved in THF (100.0 mL) and added to the previous THF solution at −70° C. The mixture was stirred at −70° C. for 2 hours. TLC (petroleum ether/ethyl acetate=5/1, reactant 1 Rf=0.1, product Rf=0.3) showed a majority of compound A5 was consumed and a main spot was formed. The mixture was quenched with aqueous NH₄Cl (500.0 mL) at −70° C. while stirring, extracted with ethyl acetate (500.0 mL, 3 times), and the organic phase was then washed with brine (600 mL, 2 times), dried over sodium sulfate, filtered and concentrated under vacuum to give a crude product. The crude product was purified by silica gel column chromatography (petroleum ether/ethyl acetate=10/1˜3/1) to give impure product (35.0 g) as a yellow solid, which was confirmed by LCMS (product Rt=0.994 min, m/z=379.0 (M+1)⁺) and was purified by reversed C₁₈ column chromatography (10%˜75% acetonitrile in water +0.1% FA). The eluent was concentrated under vacuum to remove acetonitrile and filtered, and the solid was dried under vacuum to give compound A6 (11.5 g, 30.4 mmol, 13.3% yield, 100% purity) as a white solid, which was confirmed by LCMS (product Rt=0.981 min, m/z=378.9 (M+1)⁺) and HNMR.

Compound A6 (8.00 g, 21.12 mmol, 1.00 eq) was purified by flash silica gel column chromatography (petroleum ether/ethyl acetate=5/1˜0/1) to give a purer form (7.50 g, 19.80 mmol, 93.75% yield) as a white solid, which was confirmed by HNMR and TLC (petroleum ether/ethyl acetate=5/1, compound A6 Rf=0.2).

¹H NMR (400 MHz, CDCl₃):

### δ 8.12 (s, 1H), 8.01 (s, 1H), 7.30 (s, 1H), 7.17-7.20 (m, 3H), 7.08-7.10 (m, 2H), 3.96 (s, 2H), 1.37 (s, 9H) ppm.

### Synthesis of Compound A7

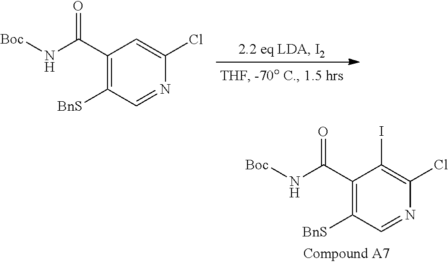

To a mixture of compound A6 (5.00 g, 13.2 mmol, 1.00 eq) in THF (130.0 mL) was added drop-wise LDA (2 M, 14.5 mL, 2.20 eq) at −70° C. and the mixture was stirred at −70° C. for 1 hour. A solution of 12 (7.37 g, 29.0 mmol, 5.85 mL, 2.20 eq) in THF (20.0 mL) was added drop-wise and the mixture was stirred at −70° C. for 30 minutes. TLC (petroleum ether:ethyl acetate=5/1, compound A6 Rf=0.2, compound A7 Rf=0.25) and LCMS (product Rt=1.036 min, m/z=505.0 (M+1)⁺) showed most of compound A6 was consumed and the desired MS was found. The mixture was quenched by aqueous NH₄Cl (100.0 mL), extracted with ethyl acetate (150.0 mL, 2 times) and the organic phase was concentrated under vacuum to give crude product. The crude product was triturated in acetonitrile (35.0 mL) to give a yellow solid confirmed by HPLC (77.1% purity), followed in ethyl acetate/petroleum ether (2/1, 26.0 mL), then filtered and the solid was collected to give 1^{st} batch of product. The filtrate was concentrated under vacuum to give a residue, which was triturated in ethyl acetate/petroleum ether (2/1, 8.00 mL) to give 2^{nd} batch of product. The two batches were combined and dried under vacuum to give compound A7 (3.50 g, 6.64 mmol, 50.3% yield, 95.8% purity) as a light yellow solid confirmed by LCMS (compound A7 Rt=1.031 min, m/z=504.8 (M+1)⁺) and HNMR. Compound A7 was also confirmed by 2D-NMR in a pilot reaction.

¹H NMR (400 MHz, CDCl₃):

### δ 7.80 (s, 1H), 7.50 (s, 1H), 7.10-6.80 (m, 5H), 3.90 (s, 2H), 1.30 (s, 9H) ppm.

### Synthesis of Compound A8

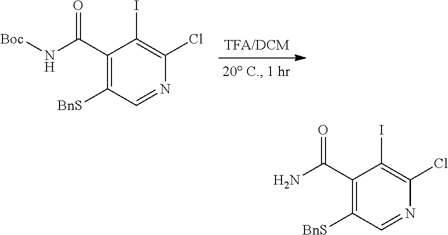

Compound A8

To a mixture of compound A7 (3.50 g, 6.93 mmol, 1.00 eq) in DCM (20.0 mL) was added TFA (6.16 g, 54.0 mmol, 4.00 mL, 7.79 eq) at 20° C. and the mixture was stirred at 20° C. for 1 h. TLC (petroleum ether:ethyl acetate=3/1, product Rf=0.2) showed compound A7 was consumed and one main spot was formed. The mixture was concentrated under vacuum to give crude product. To the crude product was added ethyl acetate (100.0 mL) and the mixture was adjusted to pH-7 with NaHCO₃, which was extracted with ethyl acetate (100 mL, 2 times) and concentrated under vacuum to give a yellow solid. The solid was triturated with petroleum ether/ethyl acetate (v/v=1/1, 12.0 mL) to give compound A8 (2.40 g, 5.29 mmol, 76.3% yield, 89.2% purity) as a yellow solid confirmed by LCMS (product Rt=0.866 min, m/z=404.7 (M+1)⁺) and HNMR.

¹H NMR (400 MHz, DMSO-d₆):

### δ 8.19 (s, 1H), 8.08 (s, 1H), 7.95 (s, 1H), 7.25-7.33 (m, 5H), 4.28 (s, 2H) ppm.

### Synthesis of Compound A9

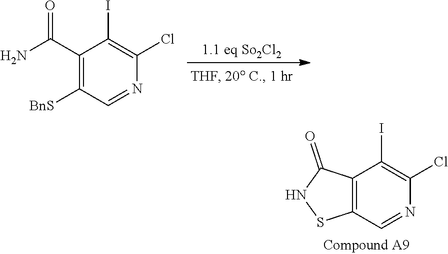

To a mixture of compound A8 (2.40 g, 5.93 mmol, 1.00 eq) in DCM (10.0 mL) was added sulfuryl chloride (880.6 mg, 6.52 mmol, 652.3 μL, 1.10 eq) at 20° C. and the mixture was stirred at 20° C. for 1 hour. LCMS (product Rt=0.777 min, m/z=313.3 (M+1)⁺) showed compound A8 was consumed and a main peak with the desired MS was formed. The mixture was concentrated under vacuum to give crude product, which was triturated in ethyl acetate (20.0 mL) and dried under vacuum to give compound A9 (1.64 g, 4.75 mmol, 80.0% yield, 90.5% purity) as a light yellow solid confirmed by LCMS (product Rt=0.682 min, m/z=312.9 (M+1)⁺), HPLC (94.3% purity) and HNMR.

¹H NMR (400 MHz, DMSO-d₆):

### δ 9.10 (s, 1H) ppm.

### Example B: Synthesis of Compounds B2 Through B6

### Synthesis of Compound B2

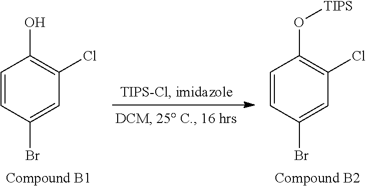

To a mixture of compound B1 (90.0 g, 433.8 mmol, 1.00 eq) in DCM (900.0 mL) was added TIPSCI (100.4 g, 520.6 mmol, 111.4 mL, 1.20 eq) and imidazole (73.8 g, 1.08 mol, 2.50 eq). The mixture was stirred at 25° C. for 16 hours. TLC (petroleum ether/ethyl acetate=3/1, compound B1 Rf=0.9, compound B2 Rf=0.9) showed compound B1 was consumed completely and one new spot was observed. The mixture was washed with brine (50.0 mL, 4 times), and the combined organic phase was dried with anhydrous Na₂SO₄, filtered, and the filtrate was concentrated to give crude compound B2 (140.3 g) as a colorless oil, which was confirmed by HNMR. Compound B2 was used for the next reaction without further purification.

¹H NMR (400 MHz, CDCl₃):

### δ 1.06 (s, 2H) 7.48 (d, J=2.4 Hz, 1H), 7.21 (dd, J=8.8, 2.4 Hz, 1H), 1.27-1.33 (m, 3H), 6.78 (d, J=8.8 Hz, 1H), 1.12 (d, J=7.2 Hz, 18H) ppm.

### Synthesis of Compound B3

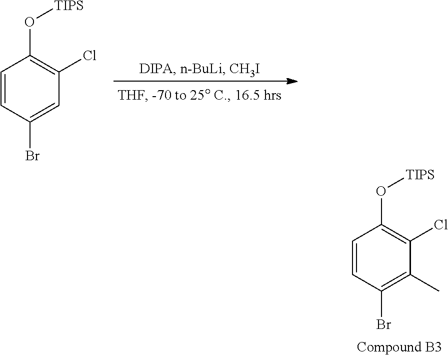

To a solution of DIPA (42.0 g, 415.6 mmol, 58.7 mL, 1.20 eq) in THF (0.5 L) was added n-BuLi (2.50 M, 152.3 mL, 1.10 eq) at −70° C. and the mixture was stirred for 30 minutes at −70° C. A solution of compound B2 (126.0 g, 346.3 mmol, 1.00 eq) in THF (1.50 L) was added dropwise to the mixture, and the mixture was stirred for 4 hours at −70° C. under nitrogen atmosphere. CH₃I (122.9 g, 865.8 mmol, 53.9 mL, 2.50 eq) was added dropwise at −70° C. and the mixture was slowly warmed up to 25° C. for 12 hours. HPLC (compound B2 Rt=2.620 min) showed the Rt of start material. HPLC (compound B3 Rt=2.776 min, compound B2 Rt=2.621 min) showed a new peak was produced. The reaction mixture was quenched by addition of NH₄Cl (1 M, 1000.0 mL), and then extracted with EtOAc (1000.0 mL, 2 times). The combined organic layers were dried over Na₂SO₄, filtered and concentrated under reduced pressure to give a residue. The residue was purified by column chromatography (SiO₂, n-hexane, Petroleum ether/Ethyl acetate=100:1, compound B3 R_{f}=0.84) to give compound B3 (121.8 g, 84.4% purity, 83.8% yield) as a colorless oil confirmed by HPLC and HNMR.

### Synthesis of Compound B4

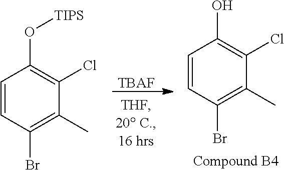

To a mixture of compound B3 (121.8 g, 270.8 mmol, 1.00 eq) in THF (500.0 mL) was added TBAF (1.00 M, 284.3 mL, 1.05 eq) at 20° C. The mixture was stirred at 20° C. for 16 hours. TLC (Petroleum ether/Ethyl acetate=3:1, compound B3 Rf=0.99, compound B4 Rf=0.6) indicated compound B3 was consumed completely and a main spot was formed. To the mixture was added saturated brine (500.0 mL) and ethyl acetate (500.0 mL). The aqueous phase was extracted with ethyl acetate (500.0 mL, 2 times). The combined organic layers were dried over anhydrous Na₂SO₄, filtered, and concentrated under vacuum to give crude compound B4. Crude compound B4 was purified by silica gel chromatography (Petroleum ether/Ethyl acetate=10:1˜4:1; TLC, Petroleum ether/Ethyl acetate=3:1, Rf=0.6) to give compound B4 (130.0 g, crude) as a yellow oil confirmed by HNMR.

¹HNMR (400 MHz, CDCl₃):

### δ (d, J=8.8 Hz, 1H), 6.80 (d, J=8.8 Hz, 1H), 5.55 (br d, J=2.4 Hz, 1H), 2.52 (s, 3H) ppm.

### Synthesis of Compound B5

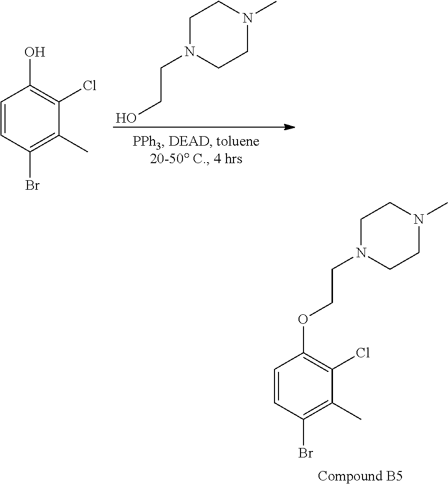

To a solution of compound B4 (45.0 g, 203.2 mmol, 1.00 eq), 2-(4-methylpiperazin-1-yl)ethan-1-ol (44.0 g, 304.8 mmol, 1.50 eq) and PPh₃ (80.0 g, 304.8 mmol, 1.50 eq) in toluene (550.0 mL) was added DEAD (70.8 g, 406.4 mmol, 73.9 mL, 2.00 eq) at 20° C. under nitrogen atmosphere. The mixture was heated to 50° C. and stirred for 2 hours, and then HCl/MeOH (4.00 M, 270.0 mL, 5.32 eq) was added to the mixture, and stirred at 20° C. for 2 hours. LCMS (compound B5 Rt=0.772 min, m/z=349.1 (M+1)⁺) showed compound B4 was consumed completely and the desired mass was detected. The reaction mixture was filtered to obtained the filtrate cake, and the cake was dissolved with H₂O/MeOH (v/v=1/1, 800.0 mL), and the pH was adjusted to pH 10 with saturated aqueous Na₂CO₃. The mixture was concentrated under reduced pressure to remove MeOH, and the solution was extracted with EtOAc (500.0 mL, 3 times). The combined organic phase was washed with brine (500.0 mL), dried with Na₂SO₄, filtered and concentrated under reduced pressure to give compound B5 as a brown oil residue (43.1 g, 107.9 mmol, 53.1% yield, 87% purity), which was confirmed by HNMR and LCMS (Compound B5 Rt=0.761 min, m/z=348.9 (M+1)⁺). Compound B5 was used next step without further purification.

¹HNMR (400 MHz, CDCl₃):

### δ 7.34-7.41 (m, 1H), 6.67 (d, J=8.8 Hz, 1H), 4.07-4.19 (m, 2H), 2.83-2.89 (m, 2H), 2.66 (br s, 4H), 2.41-2.54 (m, 7H), 2.24-2.31 (m, 3H) ppm.

### Synthesis of Compound B6

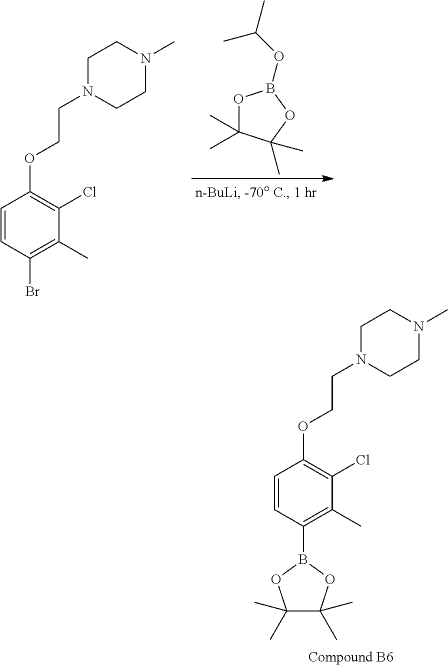

To a mixture of compound B5 (5.00 g, 14.4 mmol, 1.00 eq) and 2-isopropoxy-4,4,5,5-tetramethyl-1,3,2-dioxaborolane (5.35 g, 28.8 mmol, 5.87 mL, 2.00 eq) in THE (25.0 mL) was added n-BuLi (2.50 M, 11.5 mL, 2.00 eq) at −70° C. The mixture was stirred at −70° C. for 1 hour. LCMS (compound B6 Rt=0.974 min, m/z=395.5 (M+1)⁺) showed compound B5 was consumed completely and one main peak with the desired MS was detected. The mixture was quenched with aqueous NH₄Cl (100.0 mL), extracted with ethyl acetate (150.0 mL, 2 times), and the organic phase was dried over sodium sulfite and concentrated under vacuum to give crude compound B6. Crude compound B6 was purified by silica gel chromatography (petroleum ether/ethyl acetate=1/1-EtOH: Ethyl acetate=1/6; TLC-EtOH:Ethyl acetate=1/3, compound B6 Rf=0.1) to give compound B6 (3.08 g, 6.87 mmol, 47.8% yield, 88.1% purity) as a yellow solid confirmed by LCMS (compound B6 Rt=0.896 min, m/z=395.3 (M+1)⁺), HPLC (compound B6 Rt=2.044 min) and HNMR.

¹HNMR (400 MHz, CDCl₃):

### δ 7.64 (d, J=8.4 Hz, 1H), 6.76 (d, J=8.4 Hz, 1H), 4.18 (t, J=6.0 Hz, 2H), 2.89 (t, J=6.0 Hz, 2H), 2.68 (br s, 4H), 2.61 (s, 3H), 2.48 (br s, 4H), 2.30 (s, 3H), 1.34 (s, 12H) ppm.

### Example C: Synthesis of Compounds C2, C3 & C4, & Separation of C4-A & C4-B

### Synthesis of Compound C2

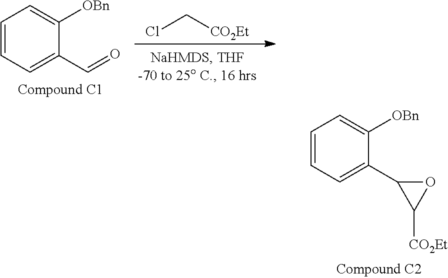 [SMILES: O=Cc1ccccc1OCc1ccccc1 ; CCOC(=O)CCl ; CCOC(=O)C1OC1c1ccccc1OCc1ccccc1]

To a mixture of compound C₁ (200.0 g, 0.94 mol, 1.00 eq) and ethyl chloroacetate (144.3 g, 1.18 mol, 125.5 mL, 1.25 eq) in THE (1000 mL) was added NaHMDS (1 M, 1.18 L, 1.25 eq) drop wise at −70° C. under nitrogen atmosphere. The reaction was stirred at −70° C. for 1 hour, then at 25° C. for 16 hours. TLC (petroleum ether/ethyl acetate=5/1, R_{f}=0.75) showed the reaction was complete. To the reaction mixture was added saturated NH₄Cl (2000 mL) and then extracted with EtOAc (400 mL, then 200 mL) to get the organic phase. The organic phase was washed with brine (200 mL) and concentrated under vacuum. The crude product mixture was purified by flash column chromatography (SiO₂, Petroleum ether/Ethyl acetate=5/1) to give compound C₂ (395.0 g, 1.32 mol, 70.3% yield) as a yellow oil.

¹HNMR (400 MHz, CDCl₃):

### δ 7.45-7.40 (m, 6H), 7.31 (d, J=4.0 Hz, 1H), 7.23-7.22 (m, 2H), 5.17 (s, 2H), 4.53 (d, J=2.0 Hz, 1H), 4.37-4.29 (m, 2H), 3.49 (s, 1H), 1.36-1.32 (m, 3H) ppm.

### Synthesis of Compound C₃

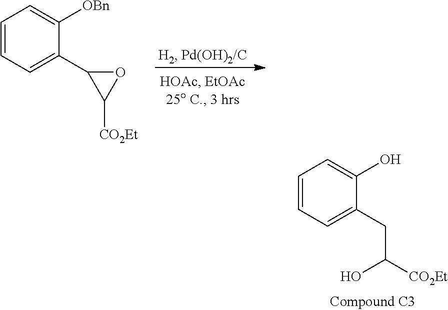 [SMILES: CCOC(=O)C1OC1c1ccccc1OCc1ccccc1 ; CCOC(=O)C(O)Cc1ccccc1O]

To a solution of HOAc (65 mL) and EtOAc (1365 mL) was added compound C2 (195.0 g, 654.0 mmol, 1.00 eq), then the Pd(OH)₂/C (19.0 g, 10% purity, 1.00 eq). The reaction was stirred at 25° C. under 50 Psi of H₂ for 3 hours. TLC (Petroleum ether/Ethy acetate=3/1, R_{f}=0.3) showed the reaction was completed. The two reaction was filtered, combined and concentrated to get the crude product. The crude product mixture was purified by flash column chromatography (SiO₂, Petroleum ether/Ethyl acetate=20/1 to 7/1) to give crude compound C₃ (207.0 g, 985.0 mmol, 75.3% yield) as a yellow oil.

### Synthesis of Compound C₄

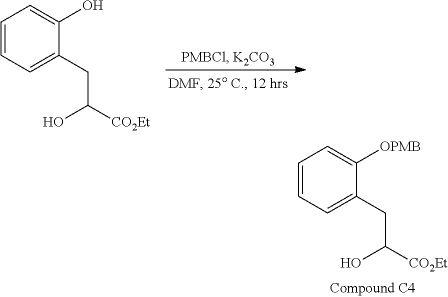

To a solution of compound C₃ (150.0 g, 713.5 mmol, 1.00 eq) in DMF (1050 mL) were added K₂CO₃ (197.2 g, 1.43 mol, 2.00 eq) and PMBCl (100.5 g, 642.0 mmol, 87.4 mL, 0.90 eq) at 25° C. TLC (Petroleum ether/Ethy acetate=2/1, R_{f}=0.4) showed the reaction was complete. The reaction mixture was quenched in ice water (1000 mL) and extracted into ethyl acetate (1000 mL, then 750 mL). Combined ethyl acetate extracts were washed with brine (500 mL) solution, dried over anhydrous sodium sulfate, filtered, and concentrated under reduced pressure. The crude product was purified by reversed-phase HPLC (Phenomenex Synergi C18 100×21.2 mm×4 μm; mobile phase: [water-ACN]; B %: 35%-57% 35 min; 57%-57%, 35 min) to give compound C4 (119.0 g, 340.0 mmol, 47.6% yield, 94.4% purity) as a yellow oil.

### Separation of Compound C4-A and C4-B

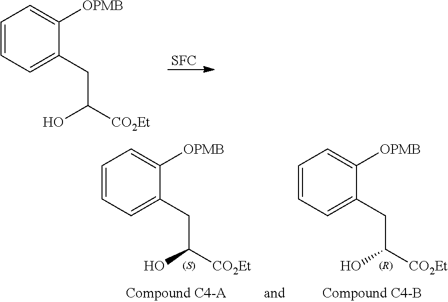

Compound C4 was separated by SFC (column: DAICEL CHIRALPAK AS (250 mm×50 mm, 10 μm); mobile phase: [Neu-ETOH]; B %: 17%-17%, 4.5 min). Both compound C4-A (27.5 g) and compound C4-B (26.4 g) gave a light yellow solid when dried. Assignment of the absolute configuration of the chiral center for compounds C4-A and C4-B was performed using vibrational circular dichroism as described in the methods section below.

### For Compound C₄-A:

¹HNMR (400 MHz, DMSO-d₆):

δ 7.37 (d, J=4.0 Hz, 2H), 7.17-7.09 (m, 2H), 7.00 (d, J=4.0 Hz, 1H), 6.92 (d, J=4.0 Hz, 2H), 6.82 (s, 1H), 5.43 (d, J=4.0 Hz, 1H), 5.01 (s, 2H), 4.23 (d, J=4.0 Hz, 1H), 3.96 (t, J=4.0 Hz, 2H), 3.73 (s, 3H), 2.97 (m, 1H), 2.75 (d, J=4.0 Hz, 1H), 1.03 (t, J=4.0 Hz, 3H) ppm.

SFC: ee value of 97.1%

HPLC: Rt=3.365 min, purity of 100.0%
LCMS: Rt=2.602 min, m/z=348.2 (M+18)⁺

### For Compound C4-B:

¹HNMR (400 MHz, DMSO-d₆):

δ 7.37 (d, J=4.0 Hz, 2H), 7.15-7.10 (m, 2H), 7.00 (d, J=4.0 Hz, 1H), 6.92 (d, J=4.0 Hz, 2H), 6.82 (s, 1H), 5.43 (d, J=4.0 Hz, 1H), 5.01 (s, 2H), 4.23 (d, J=4.0 Hz, 1H), 3.97 (t, J=4.0 Hz, 2H), 3.73 (s, 3H), 2.97 (m, 1H), 2.71-2.76 (m, 1H), 1.03 (t, J=4.0 Hz, 3H) ppm.
SFC: ee value of 98.5%
HPLC: product Rt=3.352 min, purity of 98.0%
LCMS: product Rt=2.590 min, m/z=348.2 (M+18)⁺

### Example D: Synthesis of Compounds D2, D3 & D5

### Synthesis of Compound D2

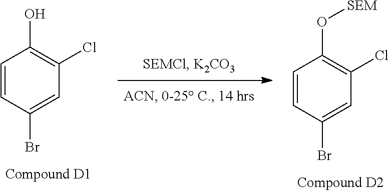 [SMILES: Oc1ccc(Br)cc1Cl ; C[Si](C)(C)CCOCOc1ccc(Br)cc1Cl]

To a mixture of compound D1 (100.0 g, 482.0 mmol, 1.00 eq) and K₂CO₃ (79.9 g, 578.45 mmol, 1.20 eq) in ACN (500 mL) was added SEMCl (141.3 g, 847.5 mmol, 150.0 mL, 1.76 eq) at 0° C., and the mixture was stirred at 25° C. for 14 hours. TLC (petroleum ether/ethyl acetate=10/1, compound D1 R_{f}=0.03, compound D2 R_{f}=0.6) showed most of compound D1 was consumed and a main spot was formed. The mixture was filtered and washed with ACN (100.0 mL). The filtrate was concentrated under vacuum to give the crude. The crude was purified by silica gel column chromatography (petroleum ether/ethyl acetate=1/0˜20/1; TLC, Petroleum ether/ethyl acetate=10/1, compound D2 R_{f}=0.6) to give compound D2 (104.0 g, 307.9 mmol, 63.9% yield) as a light-yellow oil. ¹HNMR (400 MHz, CDCl₃): δ 7.51 (d, J=2.4 Hz, 1H), 7.31 (dd, J=2.4, 8.8 Hz, 1H), 7.09 (d, J=8.8 Hz, 1H), 5.29-5.26 (m, 1H), 5.28 (s, 1H), 3.85-3.75 (m, 1H), 3.95-3.70 (m, 1H), 1.07-0.84 (m, 2H), 0.01 (s, 9H) ppm.

### Synthesis of Compound D3

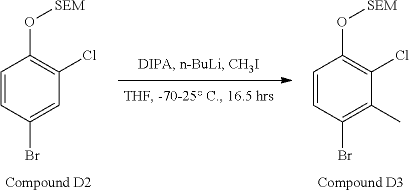 [SMILES: C[Si](C)(C)CCOCOc1ccc(Br)cc1Cl ; Cc1c(Br)ccc(OCOCC[Si](C)(C)C)c1Cl]

To a solution of DIPA (15.7 g, 155.7 mmol, 22.0 mL, 1.50 eq) in THF (140.0 mL) was added n-BuLi (2.5 M, 45.6 mL, 1.10 eq) at −70° C. The mixture was stirred at −70° C. for 30 minutes. To the mixture was added a solution of compound D2 (35.0 g, 103.6 mmol, 1.00 eq) in THF (350.0 mL) at −70° C. The mixture was stirred at −70° C. for 4 hours. To the mixture was added Mel (17.6 g, 124.3 mmol, 7.74 mL, 1.20 eq) at −70° C., and stirred at 25° C. for 12 hours. HPLC (compound D3, Rt=2.985 min) showed compound D2 was consumed completely and one main peak with new compound was detected. HPLC showed the peak of material. The reaction mixture was quenched by addition of saturated NH₄Cl (400.0 mL) at 0° C. for 30 minutes, and then extracted with EtOAc (400.0 mL, 2 times). The combined organic layers were dried over Na₂SO₄, filtered and concentrated under reduced pressure to give a residue. Compound D3 (35.0 g, 92.6 mmol, 89.4% yield, 93.1% purity) was obtained as a yellow oil.

¹HNMR (400 MHz, CDCl₃): δ 7.39 (d, J=8.8 Hz, 1H), 6.96 (d, J=8.8 Hz, 1H), 5.31-5.25 (m, 2H), 3.82-3.77 (m, 2H), 2.53 (s, 3H), 0.99-0.92 (m, 2H), 0.02-0.00 (m, 9H) ppm.

### Synthesis of Compound D5

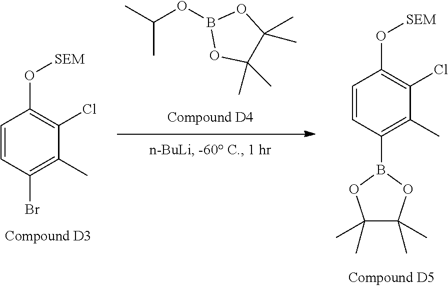

To a mixture of compound D3 (35.0 g, 92.6 mmol, 1.00 eq) and compound D4 (35.00 g, 188.1 mmol, 38.4 mL, 2.03 eq) in THE (175.0 mL) was added n-BuLi (2.5 M, 77.0 mL, 2.08 eq) at −60° C. The mixture was stirred at −60° C. for 1 hour. HPLC showed compound D3 was consumed completely and one main peak with new compound was detected. The reaction mixture was quenched by addition of saturated NH₄Cl solution (300.0 mL) at 0° C. for 20 minutes, stirred at 20° C. for 30 minutes, and extracted with ethyl acetate (200.0 mL, 2 times). The combined organic layers were washed with brine (300.0 mL), dried over Na₂SO₄, filtered and concentrated under reduced pressure to give a residue. The residue was purified by column chromatography (SiO₂, Petroleum ether/Ethyl acetate=500/1 to 50/1, Petroleum ether/Ethyl acetate=50/1, R_{f}=0.6) to give compound D5 (15.34 g, 37.0 mmol, 39.9% yield, 96.2% purity) as a yellow oil.

¹HNMR (400 MHz, CDCl₃): δ 7.64 (d, J=8.4 Hz, 1H), 7.03 (d, J=8.4 Hz, 1H), 5.32 (s, 2H), 3.86-3.74 (m, 2H), 2.63 (s, 3H), 1.35 (s, 12H), 0.98-0.93 (m, 2H), 0.01 (s, 9H) ppm.

### Example 1: Synthesis of Intermediate 1-1, 1-2, 1-3, 1-4, 1-5, Compound 1-6, Compounds 1 Through 30, and Compounds 52 and 91

### Synthesis of Compound 1-6

### Synthesis of Intermediate 1-1

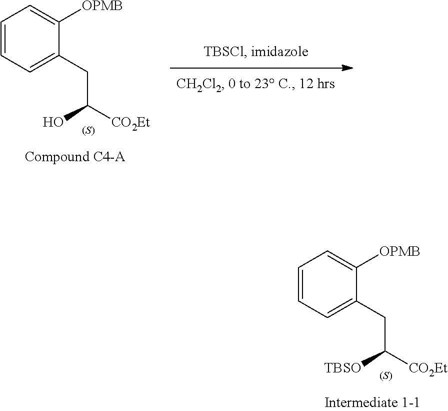

A solution of compound C4-A (2.0 g, 6.05 mmol) and imidazole (1.24 g, 18.2 mmol) in dichloromethane (20 mL) was cooled to 0° C. To this cooled solution was added tert-butyldimethylsilyl chloride (1.4 g, 9.08 mmol) in one portion. The reaction was removed from the cooling bath and stirred at ambient temperature for 12 hours at which time TLC analysis showed complete conversion to the desired product.

The reaction was stopped and poured into a separatory funnel containing water (20 mL). The organic phase was separated and the aqueous phase was washed with dichloromethane (20 mL, 2 times). The combined organic extracts were concentrated onto silica gel. Silica gel chromatography was performed (0-30% ethyl acetate/hexanes). The product fractions were pooled and concentrated to yield intermediate 1-1 as a clear oil (2.8 g, >99% yield).

¹HNMR (500 MHz, CDCl₃):

δ 7.42-7.36 (m, 2H), 7.24-7.13 (m, 2H), 6.95-6.88 (m, 3H), 6.87 (td, J=7.4, 1.1 Hz, 1H), 5.03 (s, 2H), 4.49 (dd, J=9.0, 4.8 Hz, 1H), 4.13 (q, J=7.2 Hz, 2H), 3.82 (s, 3H), 3.23 (dd, J=13.0, 4.7 Hz, 1H), 2.85 (dd, J=13.0, 9.0 Hz, 1H), 1.20 (t, J=7.1 Hz, 3H), 0.75 (s, 9H), −0.17 (s, 3H), −0.28 (s, 3H).

### Synthesis of Intermediate 1-2

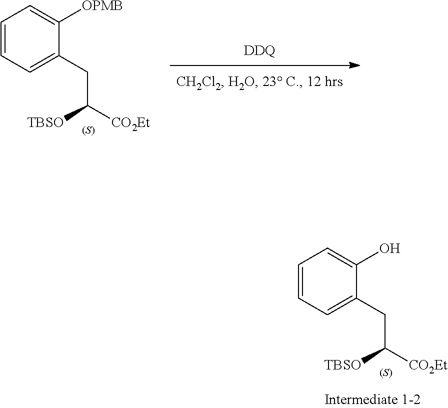

A round-bottomed flask was charged with intermediate 1-1 (1.0 g, 2.25 mmol), and dichloromethane (200 mL) and deionized water (23 mL) were added to yield a clear solution. The reaction was stirred and 2,3-dichloro-5,6-dicyano-1,4-benzoquinone (2.0 g, 8.78 mmol) was added in one portion and left to stir under nitrogen at ambient temperature for 12 hours, at which time TLC analysis showed complete conversion to the desired product.

The reaction was stopped and poured into a separatory funnel containing saturated aqueous sodium bicarbonate (50 mL). The organic phase was separated and the aqueous phase was washed with ethyl acetate (50 mL, 2 times). The combined organics were concentrated onto silica gel. Silica gel chromatography was performed (0-30% ethyl acetate/hexanes). The product fractions were pooled and concentrated to yield intermediate 1-2 as a white solid (0.569 g, 78% yield).

¹H NMR (500 MHz, CDCl₃):

δ 7.59 (s, 1H), 7.14 (td, J=7.6, 1.7 Hz, 1H), 7.02 (dd, J=7.5, 1.7 Hz, 1H), 6.90 (dd, J=8.0, 1.2 Hz, 1H), 6.81 (td, J=7.4, 1.2 Hz, 1H), 4.52 (dd, J=6.7, 3.8 Hz, 1H), 4.16-4.07 (m, 2H), 3.15-3.04 (m, 2H), 1.19 (t, J=7.2 Hz, 3H), 0.90 (s, 9H), 0.08 (s, 3H), 0.01 (s, 3H).

### Synthesis of Intermediate 1-3

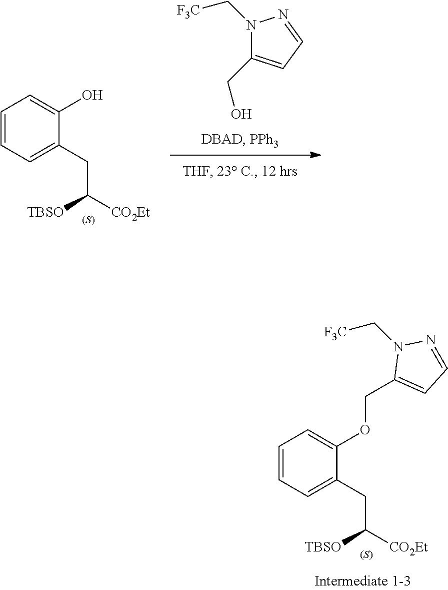

To a cooled suspension of intermediate 1-2 (2.0 g, 6.16 mmol), (1-(2,2,2-trifluoroethyl)-1H-pyrazol-5-yl)methanol (2.0 g, 11.1 mmol), and triphenylphosphine (3.2 g, 12.32 mmol) in tetrahydrofuran (50 mL) was added di-tert-butyl azodicarboxylate (2.8 g, 12.32 mmol) in tetrahydrofuran (12 mL) dropwise via cannula. The reaction was removed from the cooling bath and stirred at ambient temperature for 12 hours, at which time LC/MS analysis showed conversion to the desired product.

The reaction was concentrated onto silica gel. Silica gel chromatography was performed with refractive index detection (0-10% methanol/dichloromethane). The product fractions were pooled and concentrated to yield intermediate 1-3 as a clear oil (1.69, 56% yield).

¹H NMR (300 MHz, CDCl₃):

δ 7.58 (d, J=1.8 Hz, 1H), 7.29-7.15 (m, 2H), 7.00-6.89 (m, 2H), 6.42 (d, J=1.8 Hz, 1H), 5.14 (s, 2H), 4.90 (q, J=8.5 Hz, 2H), 4.33 (dd, J=9.1, 4.5 Hz, 1H), 4.12 (q, J=7.1 Hz, 2H), 3.13 (dd, J=13.2, 4.5 Hz, 1H), 2.81 (dd, J=13.2, 9.1 Hz, 1H), 1.20 (t, J=7.1 Hz, 3H), 0.74 (s, 9H), −0.18 (s, 3H), −0.29 (s, 3H).

### Synthesis of Intermediate 1-4

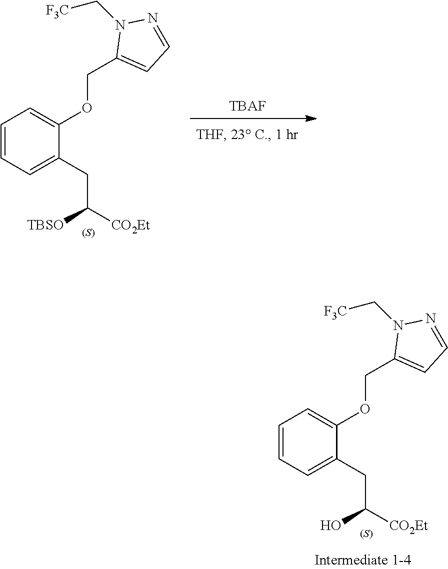

A round-bottomed flask was charged with intermediate 1-3 (1.69 g, 3.47 mmol), and dissolved in tetrahydrofuran (29 mL) to yield a clear solution. The reaction was stirred under nitrogen and tetra-n-butylammonium fluoride (4.2 mL, 1.0 M in tetrahydrofuran) was added dropwise to the stirring solution and left to stir at ambient temperature for 1 hour, at which time TLC analysis showed complete conversion to the desired product.

The reaction was stopped and poured into a separatory funnel containing saturated aqueous ammonium chloride (20 mL). The organic phase was separated and the aqueous phase was washed with ethyl acetate (50 mL, 2 times). The combined organics were concentrated onto silica gel. Silica gel chromatography was performed (0-30% acetone/hexanes). The product fractions were pooled and concentrated to yield intermediate 1-4 as a white solid (0.929 g, 72% yield).

¹H NMR (300 MHz, CDCl₃):

δ 7.58 (d, J=1.8 Hz, 1H), 7.31-7.18 (m, 2H), 7.04-6.91 (m, 2H), 6.41 (d, J=1.8 Hz, 1H), 5.13 (s, 2H), 4.90 (q, J=8.4 Hz, 2H), 4.45-4.31 (m, 1H), 4.28-4.05 (m, 2H), 3.17 (dd, J=13.8, 4.5 Hz, 1H), 2.89 (dd, J=13.8, 8.0 Hz, 1H), 1.22 (t, J=7.1 Hz, 3H).

### Synthesis of Intermediate 1-5

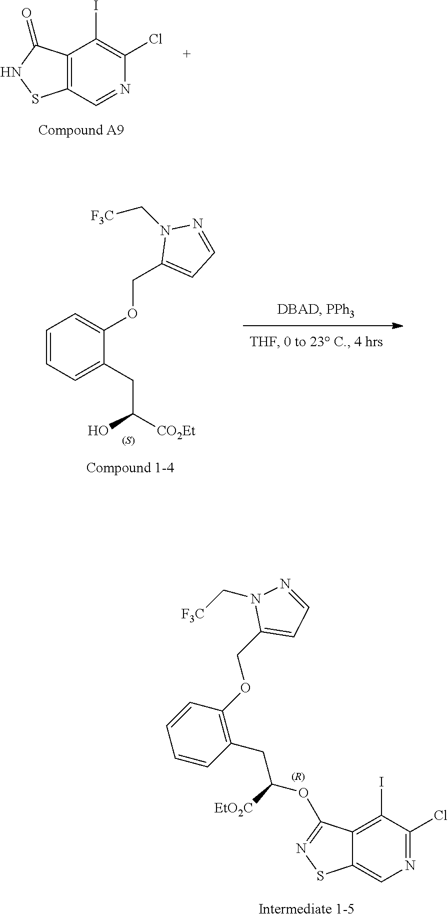

To a cooled suspension of compound A9 (700 mg, 2.24 mmol), intermediate 1-4 (876 mg, 2.4 mmol), and triphenylphosphine (881 mg, 3.36 mmol) in tetrahydrofuran (15 mL) was added di-tert-butyl azodicarboxylate (774 mg, 3.36 mmol) in tetrahydrofuran (7 mL) dropwise via cannula. The reaction was removed from the cooling bath and stirred at ambient temperature for 4 hours, at which time LC/MS analysis showed complete conversion to the desired product.

The reaction was concentrated onto silica gel. Silica gel chromatography was performed with refractive index detection (0-40% ethyl acetate/hexanes). The product fractions were pooled and concentrated to yield intermediate 1-5 as an off-white solid (650 mg, 44% yield).

¹H NMR (500 MHz, CDCl₃):

δ 8.77 (s, 1H), 7.55 (d, J=1.9 Hz, 1H), 7.39 (dd, J=7.7, 1.7 Hz, 1H), 7.30-7.25 (m, 1H), 7.00-6.93 (m, 2H), 6.40 (d, J=1.8 Hz, 1H), 5.69 (dd, J=8.7, 4.8 Hz, 1H), 5.18-5.07 (m, 2H), 4.97-4.81 (m, 2H), 4.14 (q, J=7.1 Hz, 2H), 3.46 (dd, J=14.0, 4.8 Hz, 1H), 3.32 (dd, J=14.0, 8.7 Hz, 1H), 1.14 (t, J=7.1 Hz, 3H).

### Synthesis of Compound 1-6

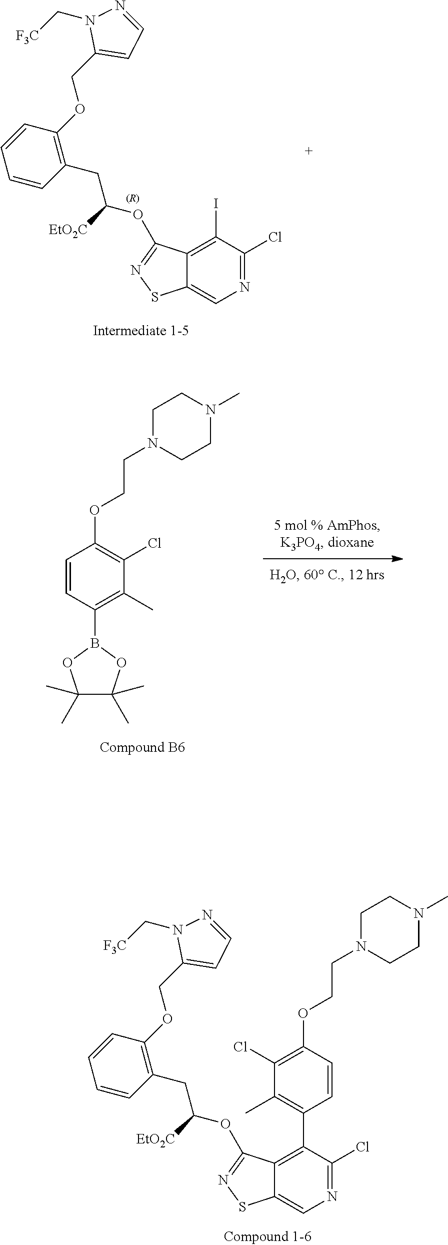

A flask containing intermediate 1-5 (350 mg, 0.526 mmol) was charged with compound B6 (250 mg, 0.633 mmol), bis(di-tert-butyl(4-dimethylaminophenyl)phosphine)dichloropalladium(II) (19 mg, 0.027 mmol), and potassium phosphate tribasic (335 mg, 1.58 mmol). The solids were dissolved in degassed 1,4-dioxane (1.75 mL) and deionized water (0.88 mL). The reaction was stirred at 60° C. for 12 hours, allowed to cool and poured into a separatory funnel containing water (3 mL). The organic phase was separated and the aqueous phase was washed with dichloromethane (5 mL, 2 times). The combined organic extracts were concentrated onto silica gel. Silica gel chromatography was performed (0-20% methanol/dichloromethane). The product fractions were pooled and concentrated to yield compound 1-6 as a yellow solid and a mixture of diastereomers (274 mg, 65% yield).

¹H NMR (500 MHz, Methanol-d₄):

δ 9.06 (s, 0.2H), 9.05 (s, 0.8H), 7.58 (d, J=1.9 Hz, 0.8H), 7.57 (d, J=1.9 Hz, 0.2H), 7.25-7.10 (m, 3H), 7.07-7.03 (m, 1H), 6.92-6.85 (m, 1H), 6.56 (d, J=1.9 Hz, 1H), 6.45 (dd, J=7.4, 1.7, 1H), 5.35 (dd, J=9.7, 3.7 Hz, 0.8H), 5.30 (dd, J=8.7, 5.1 Hz, 0.2H), 5.24-5.17 (m, 2H), 5.03 (q, J=8.6, 2H), 4.37-4.14 (m, 2H), 4.26 (td, J=5.6, 2.0 Hz, 2H), 4.19-4.08 (m, 2H), 4.11-4.02 (m, 2H), 3.09 (dd, J=13.8, 3.7, 1H), 2.96-2.87 (m, 2H), 2.57 (s, 0.5H), 2.29 (s, 0.5H), 2.25 (s, 3H), 2.18 (s, 0.5H), 2.00 (s, 3H), 1.90 (s, 0.5H), 1.34 (s, 3H), 1.10 (t, J=7.1 Hz, 2.4H), 1.04 (t, J=7.1 Hz, 0.6H).

### Synthesis of Compounds 1 and 2

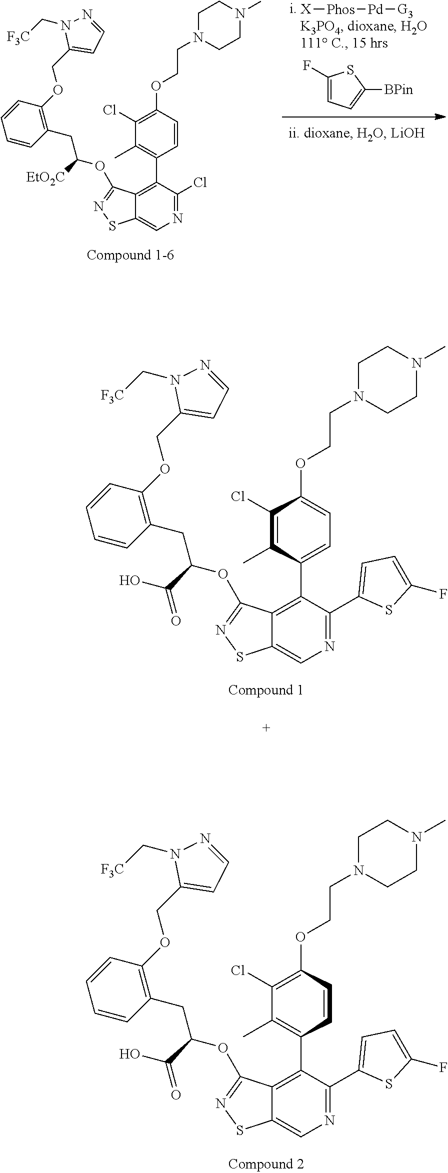

A vial was charged with compound 1-6 (80.7 mg, 0.0999 mmol), the boronate (68 μL, 0.300 mmol), X-Phos-Pd-G₃ (8.5 mg, 0.0099 mmol) and potassium phosphate (63.4 mg, 0.300 mmol). Under argon atmosphere, a solution of dioxane and water (2:1; 0.4 M) was degassed with bubbling argon. The solvent was transferred into the reaction flask, which was sealed and heated to 111° C. for a period of 15 hours. After cooling, the reaction was stirred with QuadraPure TU (Sigma-Aldrich, 200 mg) for 30 minutes. The reaction was diluted with dichloromethane/methanol and filtered through a pad of celite. The volatiles were removed under reduced pressure and the resulting solid was used directly in the saponification step.

The crude product obtained above was dissolved in a mixture of dioxane and water (2:1, 2 mL). An aqueous solution of lithium hydroxide was injected under nitrogen (1 mL, 1N) and the reaction was stirred for four hours at ambient temperature. After neutralizing with acetic acid, the mixture was filtered and purified by reversed phase HPLC (20 mm C18 Column, 25 mL/min, 25-60% acetonitrile/water+0.25% TFA, over 20 minutes). Two fractions were collected, frozen and concentrated to dryness on a lyophilizer. The resulting oily solids were converted to their HCl salts via lyophilzation of a dilute solution of aqueous hydrochloric acid. The corresponding atrope isomer fractions 1 (Compound 1, 4.01 mg) and 2 (Compound 2, 7.38 mg) were analyzed and shown to be the anticipated products by LC/MS and proton NMR.

### For Compound 1:

¹H NMR (400 MHz, DMSO-d₆):

δ 9.37 (s, 1H), 7.57 (d, J=1.8 Hz, 1H), 7.22-7.16 (m, 2H), 7.10-7.02 (m, 2H), 6.81 (t, J=7.4 Hz, 1H), 6.54 (dd, J=7.2, 2.1 Hz, 2H), 6.34 (d, J=7.4 Hz, 1H), 5.99 (t, J=4.0 Hz, 1H), 5.31-5.05 (m, 5H), 3.50-3.20 (broad, m, 8H), 3.15-3.07 (m, 1H), 2.75 (s, 3H), 2.29-2.21 (m, 1H), 2.16 (s, 3H), 1.93-1.88 (m, 1H) ppm.

LC/MS: m/z=845.3 [M+H]⁺ amu.

### For Compound 2:

¹H NMR (400 MHz, DMSO-d₆): δ 9.37 (s, 1H), 7.56 (d, J=1.8 Hz, 1H), 7.36-7.05 (m, 3H), 6.88-6.78 (m, 1H), 6.60-6.38 (m, 3H), 5.97 (t, J=4.0 Hz, 1H), 5.32-5.01 (m, 5H), 3.50-3.25 (broad, m, 8H), 3.06 (dd, J=13.9, 3.6 Hz, 1H), 2.76 (s, 2H), 2.35-2.16 (m, 2H), 1.96-1.86 (m, 1H) ppm.

LC/MS: m/z=845.3 [M+H]⁺ amu.

### Synthesis of Compounds 3 and 4

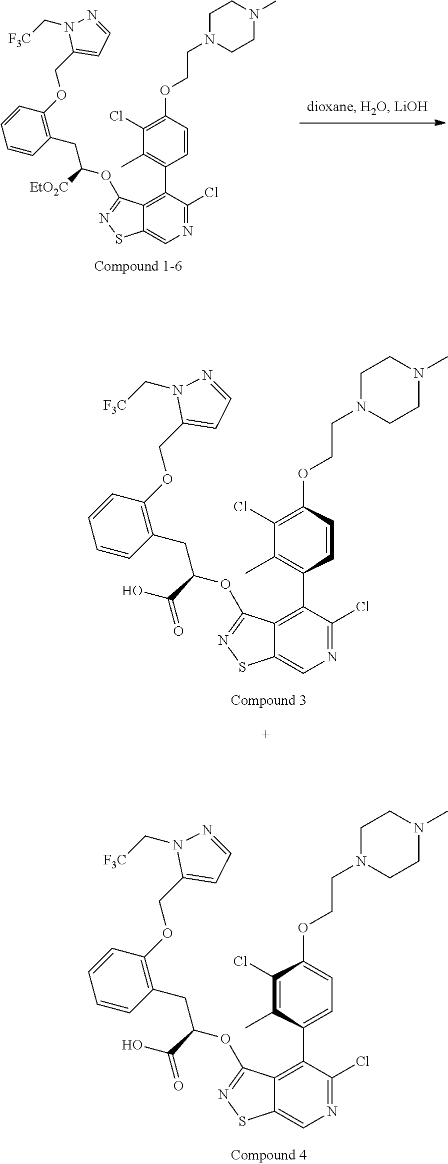

Two peaks of recovered compound 1-6 saponified to the corresponding acids were also isolated during the synthesis of compounds 1 and 2, and were analyzed and shown to be the anticipated products by LC/MS and proton NMR.

### For Compound 3:

¹H NMR (400 MHz, DMSO-d₆):

δ 9.28 (s, 1H), 7.56 (d, J=1.8 Hz, 1H), 7.28-7.13 (m, 2H), 7.08 (t, J=7.2 Hz, 2H), 6.79 (t, J=7.4 Hz, 1H), 6.52 (d, J=1.8 Hz, 1H), 6.22 (d, J=7.4 Hz, 1H), 5.28-5.08 (m, 5H), 3.50-3.30 (broad, m, 8H), 3.20-3.06 (m, 1H), 2.76 (s, 4H), 2.30-2.21 (m, 2H), 2.13 (s, 3H), 1.94-1.88 (m, 1H) ppm.

LC/MS: m/z=797.3 [M+H]⁺ amu.

### For Compound 4:

¹H NMR (400 MHz, DMSO-d₆):

δ 9.28 (s, 1H), 7.57 (d, J=1.8 Hz, 1H), 7.28-7.16 (m, 3H), 7.08 (d, J=8.3 Hz, 1H), 6.88-6.73 (m, 1H), 6.53 (d, J=1.8 Hz, 1H), 6.27 (dd, J=7.5, 1.7 Hz, 1H), 5.31-5.09 (m, 5H), 4.47 (s, 2H), 3.35-3.25 (broad, m, 8H), 3.12 (dd, J=13.8, 3.1 Hz, 1H), 2.79 (s, 3H), 2.36-2.20 (m, 2H) ppm.

LC/MS: m/z=797.3 [M+H]⁺ amu.

### Synthesis of Compounds 5 and 6

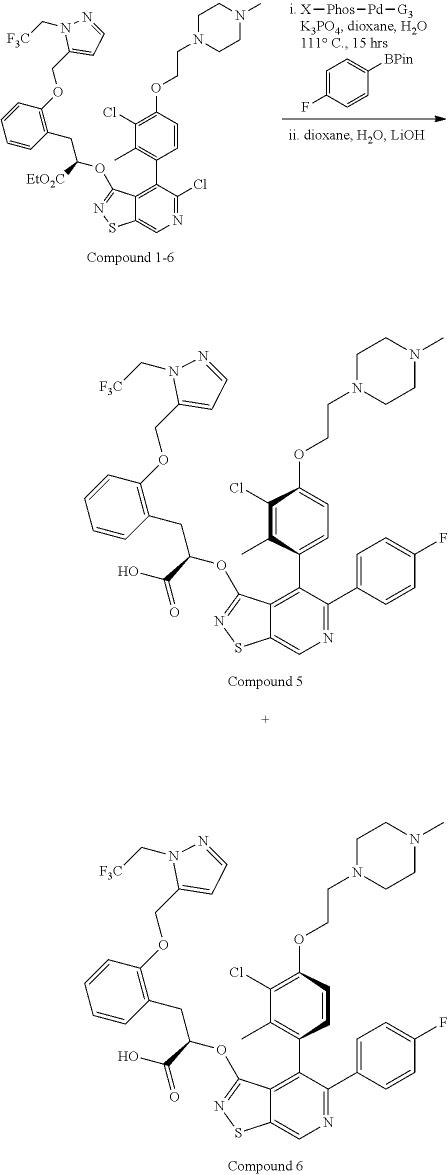

A vial was charged with compound 1-6 (75.8 mg, 0.0938 mmol), the boronate (26.3 mg, 0.188 mmol), X-Phos-Pd-G₃ (7.9 mg, 0.0094 mmol) and potassium phosphate (60.0 mg, 0.282 mmol). Under argon atmosphere, a solution of dioxane and water (2:1; 0.4 M) was degassed with bubbling argon. The solvent was transferred into the reaction flask, which was sealed and heated to 111° C. for 15 hours. After cooling, the reaction was stirred with QuadraPure TU (Sigma-Aldrich, 200 mg) for 30 minutes. The reaction was diluted with dichloromethane/methanol and filtered through a pad of celite. The volatiles were removed under reduced pressure and the resulting solid was used directly in the saponification step.

The crude product obtained above was dissolved in a mixture of dioxane and water (2:1, 4 mL). An aqueous solution of lithium hydroxide was injected under nitrogen atmosphere (2 mL, 1 N) and the reaction was stirred for four hours at ambient temperature. After neutralizing with acetic acid, the mixture was filtered and purified by reversed phase HPLC (20 mm C₁₈ Column, 25 mL/min, 25-50% acetonitrile/water+0.25% TFA, over 20 minutes). Two fractions were collected, frozen and concentrated to dryness on a lyophilizer. The resulting oily solids were converted to their HCl salts via lyophilzation of a dilute solution of aqueous hydrochloric acid. The corresponding atrope isomer fractions 1 (Compound 5, 2.1 mg) and 2 (Compound 6, 11.1 mg) were analyzed and shown to be the anticipated products by LC/MS and proton NMR.

### Compound 5:

¹H NMR (400 MHz, DMSO-d₆):

δ 9.21 (s, 1H), 8.02-7.98 (m, 1H), 7.65-7.55 (m, 1H), 7.50-7.40 (m, 2H), 7.35-7.30 (m, 2H), 7.17-7.10 (m, 1H), 7.00-7.93 (m, 2H), 6.65-6.53 (m, 1H), 6.35-6.30 (s, 1H), 5.38-5.30 (m, 1H), 5.10-4.90 (m, 4H), 4.20-4.00 (m, 2H), 3.70-3.40 (m, 10H), 3.27 (s, 3H) ppm.

LC/MS: m/z=839.2 [M+H]⁺ amu.

### Compound 6:

¹H NMR (400 MHz, DMSO-d₆): δ 9.49 (s, 1H), 7.57 (d, J=1.8 Hz, 1H), 7.40-7.23 (m, 3H), 7.22-7.13 (m, 2H), 7.11-7.05 (m, 3H), 6.79-6.65 (m, 1H), 6.53 (d, J=1.8 Hz, 1H), 6.29-6.15 (m, 1H), 5.30-5.04 (m, 5H), 3.50-3.40 (broad, m, 8H), 3.08 (dd, J=13.8, 3.5 Hz, 1H), 2.75 (s, 2H) ppm.

LC/MS: m/z=839.2 [M+H]⁺ amu.

### Synthesis of Compound 7

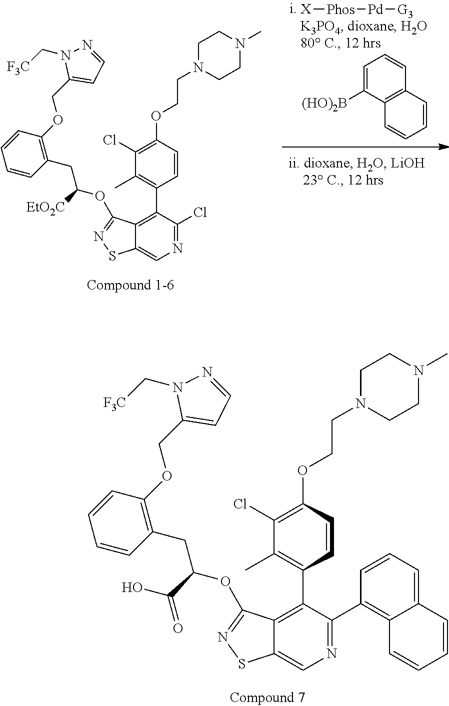

A microwave vial containing Compound 1-6 (20 mg, 0.025 mmol) was charged with X-Phos-Pd-G₃ (4 mg, 0.005 mmol), 1-naphthylboronic acid (17 mg, 0.099 mmol), and potassium phosphate tribasic (18 mg, 0.085 mmol). The vial was capped and after 3 cycles of nitrogen/vacuum, the solids were dissolved in degassed dioxane (0.333 mL, 0.08M) and water (0.167 mL, 0.15M). The reaction was stirred at 80° C. for 12 hours, and allowed to cool to room temperature. The reaction was quenched with water (1.0 mL) and transferred to a separatory funnel with dichloromethane (2 mL). The aqueous layer was extracted with dichloromethane (2 mL, 3 times). The combined organics were dried over sodium sulfate, filtered, and concentrated in vacuo.

To this crude reaction mixture was then added dioxane (0.600 mL, 0.04M) and lithium hydroxide in water (2M solution, 0.600 mL). The reaction was allowed to stir at room temperature for 12 hours. The reaction was quenched with 2N hydrochloric acid (0.300 mL), diluted with water, and transferred to a separatory funnel with chloroform/isopropyl alcohol (5:1) mixture. The aqueous layer was extracted with chloroform/isopropyl alcohol (5:1) (10 mL, 3 times). The combined organics were dried over sodium sulfate, filtered, and concentrated in vacuo. The crude reaction was purified via reverse phase chromatography (0.25% TFA/water in 20-70% acetonitrile). The product fractions were pooled and concentrated to yield Compound 7 as an off-white amorphous solid (6.7 mg, 31% yield).

¹H NMR (500 MHz, Methanol-d₄): δ 9.36 (s, 1H), 7.89-7.82 (m, 1H), 7.80 (d, J=8.1 Hz, 1H), 7.57 (d, J=1.9 Hz, 1H), 7.50-7.38 (m, 2H), 7.38-7.12 (m, 2H), 7.12-7.00 (m, 1H), 6.99-6.70 (m, 1H), 6.56 (d, J=2.5, 1H), 5.51-5.37 (m, 1H), 5.30-5.17 (m, 2H), 5.05 (qd, J=8.6, 3.2 Hz, 2H), 3.27-2.90 (m, 4H), 2.8 (s, 3H), 2.56-2.21 (m, 1H), 2.19-1.84 (m, 1H), 1.82-1.48 (m, 1H), 1.29 (s, 1H) (27 of 42 protons observed) ppm. LC/MS: m/z=871.3 [M+H]⁺ amu.

### Synthesis of Compound 8

Compound 8 was synthesized with the general procedures used for Compound 7 and using the corresponding heteroaryl boronate ester or acid. Compound 8 was obtained as an off-white solid.

LCMS: m/z=877.2 [M+H]⁺ amu.

### Synthesis of Compound 9

Compound 9 was synthesized with the general procedures used for Compound 7 and using the corresponding heteroaryl boronate ester or acid. Compound 9 was obtained as an off-white solid.

¹H NMR (500 MHz, Methanol-d₄): δ 9.35 (d, J=11.9 Hz, 1H), 8.25-8.18 (m, 1H), 8.16-8.11 (m, 1H), 8.08 (dt, J=7.8, 1.0 Hz, 3H), 7.90-7.87 (m, 1H), 7.57 (dd, J=13.2, 1.9 Hz, 1H), 7.42-7.39 (m, 1H), 7.32-7.24 (m, 1H), 7.19 (ddd, J=8.0, 7.1, 1.1 Hz, 2H), 7.14-7.09 (m, 1H), 7.01-6.98 (m, 1H), 6.79-6.67 (m, 2H), 6.56 (dd, J=27.7, 2.0 Hz, 1H), 6.49-6.41 (m, 2H), 5.42-5.31 (m, 1H), 5.30-5.22 (m, 2H), 5.17-5.05 (m, 2H), 3.80-3.73 (m, 1H), 3.07-2.74 (m, 8H), 2.63 (d, J=2.4 Hz, 3H), 2.24 (d, J=5.4 Hz, 3H) ppm.

LCMS: m/z=927.2 [M+H]⁺ amu.

### Synthesis of Compound 10

Compound 10 was synthesized with the general procedures used for Compound 7 and using the corresponding heteroaryl boronate ester or acid. Compound 10 was obtained as an off-white solid.

LCMS: m/z=874.3 [M+H]⁺ amu.

### Synthesis of Compound 11

Compound 11 was synthesized with the general procedures used for Compound 7 and using the corresponding aryl boronate ester or acid. Compound 11 was obtained as an off-white solid.

LCMS: m/z=834.3 [M+H]⁺ amu.

### Synthesis of Compound 12

Compound 12 was synthesized with the general procedures used for Compound 7 and using the corresponding heterocyclyl boronate ester or acid. Compound 12 was obtained as an off-white solid.

¹H NMR (500 MHz, Methanol-d₄): δ 9.39 (s, 1H), 9.34 (s, 1H), 7.63 (s, 1H), 7.50 (m, 2H), 7.27 (d, J=8.8 Hz, 2H), 7.10 (m, 1H), 6.98 (s, 1H), 6.85 (s, 1H), 6.61 (s, 1H), 6.37 (m, 1H), 5.29 (s, 1H), 5.13 (m, 2H), 3.33-3.03 (m, 4H), 2.91-2.84 (m, 7H), 2.72 (s, 3H), 2.1 (s, 3H) ppm.

LCMS: m/z=876.3 [M+H]⁺ amu.

### Synthesis of Compound 13

Compound 13 was synthesized with the general procedures used for Compound 7 and using the corresponding cycloalkyl boronate ester or acid. Compound 13 was obtained as an off-white solid.

¹H NMR (500 MHz, Methanol-d₄): δ 9.34 (s, 1H), 7.63 (d, J=1.9 Hz, 1H), 7.34 (d, J=8.5 Hz, 1H), 7.27-7.16 (m, 2H), 7.08 (dd, J=18.9, 8.4 Hz, 2H), 6.97 (t, J=7.5 Hz, 1H), 6.87 (td, J=7.4, 1.0 Hz, 1H), 6.81 (d, J=7.4 Hz, 1H), 6.61 (d, J=1.9 Hz, 1H), 6.45 (dd, J=7.5, 1.7 Hz, 1H), 5.47 (dd, J=9.8, 3.6 Hz, 1H), 5.29 (d, J=3.4 Hz, 2H), 5.15-5.04 (m, 3H), 4.39-4.28 (m, 2H), 3.32-3.07 (m, 5H), 2.96 (t, J=7.1 Hz, 3H), 2.88 (s, 3H), 2.45 (dd, J=13.9, 9.7 Hz, 1H), 2.14-1.97 (m, 2H), 1.85 (s, 3H) ppm. LCMS: m/z=861.2 [M+H]⁺ amu.

### Synthesis of Compound 14

Compound 14 was synthesized with the general procedures used for Compound 7 and using the corresponding heteroaryl boronate ester or acid. Compound 14 was obtained as an off-white solid.

¹H NMR (500 MHz, Methanol-d₄): δ 9.39 (s, 1H), 7.95 (s, 1H), 7.73 (s, 1H), 7.63 (d, J=1.9 Hz, 1H), 7.35 (d, J=8.5 Hz, 1H), 7.28-7.07 (m, 2H), 6.99 (d, J=8.6 Hz, 1H), 6.96-6.88 (m, 2H), 6.85 (td, J=7.5, 1.0 Hz, 1H), 6.44 (dd, J=7.5, 1.7 Hz, 1H), 5.48 (dd, J=9.6, 3.6 Hz, 1H), 5.33-5.20 (m, 2H), 5.10 (qd, J=8.5, 2.4 Hz, 2H), 4.32 (s, 2H), 3.72 (s, 3H), 3.37 (p, J=1.6 Hz, 5H), 3.22 (t, J=3.9 Hz, 1H), 2.90 (d, J=1.1 Hz, 3H), 2.49 (dd, J=13.9, 9.6 Hz, 1H), 1.82 (s, 3H) ppm.

LCMS: m/z=879.3 [M+H]⁺ amu.

### Synthesis of Compound 15

Compound 15 was synthesized with the general procedures used for Compound 7 and using the corresponding heteroaryl boronate ester or acid. Compound 15 was obtained as an off-white solid.

¹H NMR (300 MHz, Methanol-d₄): δ 9.35 (s, 1H), 9.05 (s, 1H), 7.57 (t, J=2.0 Hz, 2H), 7.32 (dd, J=8.4, 4.8 Hz, 2H), 7.25-7.12 (m, 2H), 7.01 (dd, J=22.6, 8.8 Hz, 2H), 6.95-6.85 (m, 1H), 6.75 (dd, J=13.6, 7.4 Hz, 1H), 6.54 (d, J=8.3 Hz, 1H), 6.40 (d, J=7.5 Hz, 1H), 5.43-5.31 (m, 1H), 5.22 (d, J=5.9 Hz, 3H), 5.08-5.01 (m, 2H), 3.81 (d, J=2.5 Hz, 2H), 3.20-3.01 (m, 8H), 2.99 (s, 3H) ppm.

LCMS: m/z=874.3 [M+H]⁺ amu.

### Synthesis of Compound 16

Compound 16 was synthesized with the general procedures used for Compound 7 and using the corresponding heterocyclyl boronate ester or acid. Compound 16 was obtained as an off-white solid.

¹H NMR (300 MHz, Methanol-d₄) δ 9.35 (s, 1H), 7.57 (d, J=1.9 Hz, 1H), 7.35 (dd, J=21.8, 8.4 Hz, 2H), 7.21-7.12 (m, 1H), 7.11-6.91 (m, 2H), 6.86-6.68 (m, 3H), 6.56 (d, J=1.9 Hz, 1H), 6.37 (d, J=6.2 Hz, 1H), 5.41 (dd, J=9.8, 3.7 Hz, 2H), 5.23 (s, 2H), 5.11-5.00 (m, 2H), 4.24 (t, J=4.8 Hz, 2H), 3.26-2.94 (m, 8H), 2.81 (s, 3H), 1.70 (s, 3H) ppm.

LCMS: m/z=928.3 [M+H]⁺ amu.

### Synthesis of Compound 17

Compound 17 was synthesized with the general procedures used for Compound 7 and using the corresponding heterocyclyl boronate ester or acid. Compound 17 was obtained as an off-white solid.

¹H NMR (500 MHz, Methanol-d₄): δ 9.39 (s, 1H), 7.73 (d, J=2.3 Hz, 1H), 7.63 (d, J=1.9 Hz, 1H), 7.35 (d, J=8.5 Hz, 1H), 7.23 (ddd, J=9.2, 7.6, 1.8 Hz, 1H), 7.16 (dd, J=8.3, 5.1 Hz, 1H), 7.11-7.07 (m, 1H), 6.99 (d, J=8.6 Hz, 1H), 6.95-6.89 (m, 1H), 6.85 (td, J=7.5, 1.0 Hz, 1H), 6.61 (d, J=1.9 Hz, 1H), 6.44 (dd, J=7.5, 1.7 Hz, 1H), 5.48 (dd, J=9.6, 3.6 Hz, 1H), 5.29 (d, J=4.0 Hz, 2H), 5.10 (qd, J=8.5, 2.4 Hz, 2H), 4.32 (s, 2H), 3.72 (s, 3H), 3.35-3.13 (m, 8H), 2.90 (d, J=1.1 Hz, 3H), 1.82 (s, 3H) ppm.

LCMS: m/z=874.3 [M+H]⁺ amu.

### Synthesis of Compound 18

Compound 18 was synthesized with the general procedures used for Compound 7 and using the corresponding heterocyclyl boronate ester or acid. Compound 18 was obtained as an off-white solid.

¹H NMR (500 MHz, Methanol-d₄): δ 9.38 (s, 1H), 8.03 (s, 1H), 7.63 (d, J=1.9 Hz, 1H), 7.53 (dd, J=7.8, 1.1 Hz, 1H), 7.41 (d, J=8.5 Hz, 1H), 7.28 (d, J=3.2 Hz, 1H), 7.21 (d, J=8.6 Hz, 1H), 7.06 (dd, J=23.5, 8.4 Hz, 1H), 6.88-6.79 (m, 1H), 6.77-6.69 (m, 1H), 6.62 (d, J=1.9 Hz, 1H), 6.52 (d, J=3.2 Hz, 1H), 6.41 (d, J=6.8 Hz, 1H), 5.46 (dd, J=9.9, 3.4 Hz, 1H), 5.34-5.24 (m, 2H), 5.16-5.06 (m, 2H), 3.31-3.21 (m, 8H), 3.08 (s, 2H), 3.05 (d, J=0.5 Hz, 3H), 1.72 (s, 3H) ppm.

LCMS: m/z=860.3 [M+H]⁺ amu.

### Synthesis of Compound 19

Compound 19 was synthesized with the general procedures used for Compound 7 and using the corresponding heteroaryl boronate ester or acid. Compound 19 was obtained as an off-white solid.

¹H NMR (300 MHz, Methanol-d₄): δ 9.35 (s, 1H), 7.89-7.83 (m, 1H), 7.76 (dd, J=6.7, 2.5 Hz, 1H), 7.57 (d, J=1.9 Hz, 1H), 7.38-7.31 (m, 3H), 7.20-7.12 (m, 1H), 7.10 (s, 1H), 7.03 (dd, J=8.4, 4.0 Hz, 2H), 6.78 (t, J=7.5 Hz, 1H), 6.56 (d, J=1.9 Hz, 1H), 6.40-6.31 (m, 1H), 5.41 (dd, J=9.9, 3.3 Hz, 1H), 5.23 (s, 2H), 5.11-5.01 (m, 2H), 4.28 (d, J=4.8 Hz, 2H), 3.28-2.94 (m, 8H), 2.81 (s, 3H), 1.69 (s, 3H) ppm.

LCMS: m/z=877.3 [M+H]⁺ amu.

### Synthesis of Compound 20

Compound 20 was synthesized with the general procedures used for Compound 7 and using the corresponding heteroaryl boronate ester or acid. Compound 20 was obtained as an off-white solid.

¹H NMR (300 MHz, Methanol-d₄): δ 9.46 (s, 1H), 7.77 (d, J=8.0 Hz, 1H), 7.57 (dd, J=3.4, 1.9 Hz, 1H), 7.43 (dd, J=12.1, 8.4 Hz, 2H), 7.30-7.11 (m, 3H), 7.10-6.99 (m, 1H), 6.91-6.78 (m, 1H), 6.76 (d, J=9.9 Hz, 1H), 6.56 (d, J=2.0 Hz, 1H), 6.36 (dd, J=7.5, 1.7 Hz, 1H), 5.43 (dd, J=10.0, 3.2 Hz, 1H), 5.23 (s, 2H), 5.12-5.00 (m, 2H), 4.51-4.40 (m, 2H), 3.69 (s, 3H), 3.42 (s, 8H), 2.88 (s, 3H), 1.72 (s, 3H) ppm.

LCMS: m/z=874.3 [M+H]⁺ amu.

### Synthesis of Compound 21

Compound 21 was synthesized with the general procedures used for Compound 7 and using the corresponding heteroaryl boronate ester or acid. Compound 21 was obtained as an off-white amorphous solid (10.0 mg, 31% yield).

¹H NMR (400 MHz, Methanol-d₄): δ 9.34 (s, 1H), 7.64 (d, J=2.2 Hz, 1H), 7.60-7.51 (m, 2H), 7.30 (d, J=8.6 Hz, 1H), 7.25-7.07 (m, 3H), 7.07-7.01 (m, 1H), 6.91 (d, J=8.6 Hz, 1H), 6.84-6.75 (m, 2H), 6.56 (d, J=1.9 Hz, 1H), 6.39 (dd, J=7.5, 1.7 Hz, 1H), 5.42 (dd, J=9.6, 3.7 Hz, 1H), 5.30-5.18 (m, 2H), 5.05 (qd, J=8.7, 1.5 Hz, 2H), 4.23 (t, J=4.9 Hz, 2H), 3.29-3.17 (m, 5H), 3.17-2.93 (m, 7H), 2.82 (s, 3H), 2.48-2.36 (m, 1H), 1.76 (s, 3H) ppm (40 of 40 protons observed).

LC/MS: m/z=861.2 [M+H]⁺ amu.

### Synthesis of Compound 22

Compound 22 was synthesized with the general procedures used for Compound 7 and using the corresponding aryl boronate ester or acid. Compound 22 was obtained as an off-white amorphous solid (6.7 mg, 20% yield).

¹H NMR (400 MHz, Methanol-d₄): δ 9.33 (s, 1H), 7.65-7.60 (m, 1H), 7.60-7.52 (m, 2H), 7.52-7.34 (m, 3H), 7.22-7.09 (m, 2H), 7.07-7.00 (m, 1H), 6.87-6.70 (m, 1H), 6.56 (d, J=1.9 Hz, 1H), 6.33 (dd, J=7.5, 1.7 Hz, 2H), 5.41 (dd, J=10.0, 3.3 Hz, 1H), 5.29-5.19 (m, 2H), 5.10-5.00 (m, 2H), 4.39-4.27 (m, 2H), 3.30-3.15 (m, 6H), 3.15-2.86 (m, 5H), 2.84 (s, 3H), 2.45-2.32 (m, 1H), 1.69 (s, 3H) ppm (39 of 39 protons observed).

LC/MS: m/z=889.2 [M+H]⁺ amu.

### Synthesis of Compound 23

Compound 23 was synthesized with the general procedures used for Compound 7 and using the corresponding aryl boronate ester or acid. Compound 23 was obtained as an off-white solid.

¹H NMR (500 MHz, Methanol-d₄): δ 9.10 (s, 1H), 7.62 (d, J=1.9 Hz, 1H), 7.31-7.23 (m, 4H), 7.21 (d, J=8.6 Hz, 1H), 7.15 (m, 3H), 7.11 (dd, J=8.3, 1.1 Hz, 1H), 6.93 (td, J=7.4, 1.0 Hz, 1H), 6.61 (d, J=1.9 Hz, 1H), 6.45 (dd, J=7.4, 1.7 Hz, 1H), 5.44 (dd, J=10.2, 3.0 Hz, 1H), 5.33-5.25 (m, 2H), 5.09 (td, J=8.7, 2.6 Hz, 2H), 4.46 (t, J=4.9 Hz, 2H), 4.10 (s, 3H), 3.47-3.38 (m, 4H), 3.34-3.12 (m, 4H), 2.93 (s, 3H), 2.44-2.32 (m, 1H), 2.06 (s, 3H) ppm.

LCMS: m/z=851.2 [M+H]⁺ amu.

### Synthesis of Compound 24

Compound 24 was synthesized with the general procedures used for Compound 7 and using the corresponding heteroaryl boronate ester or acid. Compound 24 was obtained as an off-white amorphous solid (12.4 mg, 38% yield).

¹H NMR (500 MHz, Methanol-d₄): δ 9.34 (s, 1H), 7.75 (dd, J=7.9, 1.1 Hz, 1H), 7.60-7.54 (m, 2H), 7.39 (d, J=5.5 Hz, 1H), 7.33 (d, J=8.6 Hz, 1H), 7.22-7.09 (m, 2H), 7.07-7.00 (m, 1H), 7.00-6.91 (m, 2H), 6.79 (td, J=7.4, 1.1 Hz, 1H), 6.56 (d, J=1.9 Hz, 1H), 6.40 (dd, J=7.5, 1.7 Hz, 1H), 5.43 (dd, J=9.7, 3.6 Hz, 1H), 5.29-5.18 (m, 2H), 5.05 (qd, J=8.6, 1.4 Hz, 2H), 4.31-4.21 (m, 2H), 3.31-3.19 (m, 4H), 3.20-2.99 (m, 6H), 2.82 (s, 3H), 2.41 (dd, J=14.0, 9.7 Hz, 1H), 1.69 (s, 3H) ppm (38 of 40 protons observed).

LC/MS: m/z=877.2 [M+H]⁺ amu.

### Synthesis of Compound 25

Compound 25 was synthesized with the general procedures used for Compound 7 and using the corresponding cycloalkyl boronate ester or acid. Compound 25 was obtained as an off-white amorphous solid (6.2 mg, 19% yield).

¹H NMR (400 MHz, Methanol-d₄): δ 9.31 (s, 1H), 7.57 (d, J=1.9 Hz, 1H), 7.41-7.14 (m, 1H), 7.14-6.68 (m, 6H), 6.68-6.35 (m, 2H), 5.55-5.33 (m, 1H), 5.33-5.15 (m, 2H), 5.04 (qd, J=8.6, 2.4 Hz, 2H), 4.43-4.11 (m, 2H), 3.30-2.93 (m, 11H), 2.93-2.68 (m, 5H), 2.66 (s, 1H), 2.60-2.11 (m, 1H), 2.09-1.85 (m, 1H), 1.85-1.64 (m, 3H) ppm (40 of 46 protons observed).

LC/MS: m/z=875.3 [M+H]⁺ amu.

### Synthesis of Compound 26

Compound 26 was synthesized with the general procedures used for Compound 7 and using the corresponding aryl boronate ester or acid. Compound 26 was obtained as an off-white amorphous solid (10.5 mg, 34% yield).

¹H NMR (400 MHz, Methanol-d₄): δ 9.30 (s, 1H), 7.57 (d, J=1.9 Hz, 1H), 7.38 (d, J=8.5 Hz, 1H), 7.30-7.21 (m, 1H), 7.21-7.14 (m, 1H), 7.14-7.07 (m, 2H), 7.07-6.90 (m, 3H), 6.80 (td, J=7.5, 1.1 Hz, 1H), 6.56 (d, J=1.9 Hz, 1H), 6.37 (dd, J=7.5, 1.8 Hz, 1H), 5.41 (dd, J=9.9, 3.4 Hz, 1H), 5.26-5.20 (m, 2H), 5.05 (qd, J=8.7, 1.7 Hz, 2H), 4.44-4.30 (m, 2H), 3.28-3.07 (m, 7H), 2.85 (s, 3H), 2.45-2.35 (m, 1H), 1.74 (s, 3H) ppm (34 of 39 protons observed).

LC/MS: m/z=839.2 [M+H]⁺ amu.

### Synthesis of Compound 27

Compound 27 was synthesized with the general procedures used for Compound 7 and using the corresponding heteroaryl boronate ester or acid. Compound 27 was obtained as an off-white amorphous solid.

¹H NMR (500 MHz, Acetonitrile-d₃): δ 9.15 (s, 1H), 7.50 (s, 1H), 7.30 (d, J=8.2 Hz, 1H), 7.20 (dd, J=4.9, 3.1 Hz, 1H), 7.13-7.03 (m, 4H), 7.00 (d, J=8.5 Hz, 1H), 6.91 (d, J=8.2 Hz, 1H), 6.73 (t, J=7.4 Hz, 1H), 6.52 (s, 1H), 6.43 (s, 1H), 5.22 (d, J=9.3 Hz, 1H), 5.16-4.93 (m, 4H), 4.29-4.14 (m, 2H), 3.73-3.69 (m, 2H), 3.68-3.64 (m, 2H), 3.61-3.57 (m, 2H), 3.53-3.50 (m, 2H), 3.23-3.11 (m, 1H), 2.88 (s, broad, 2H), 2.35 (s, 3H), 1.79 (s, 3H) ppm.

LC/MS: m/z=827.2 [M+H]⁺ amu.

### Synthesis of Compound 28

Compound 28 was synthesized with the general procedures used for Compound 7 and using the corresponding heteroaryl boronate ester or acid. Compound 28 was obtained as an off-white amorphous solid.

¹H NMR (400 MHz, Methanol-d₄): δ 9.19 (d, J=1.8 Hz, 1H), 7.57 (d, J=8.7 Hz, 2H), 7.30 (d, J=8.5 Hz, 1H), 7.19 (t, J=7.5 Hz, 2H), 7.05 (d, J=8.2 Hz, 1H), 6.90-6.80 (m, 3H), 6.55 (d, J=2.0 Hz, 1H), 6.47 (d, J=7.3 Hz, 1H), 5.40 (dd, J=9.7, 3.2 Hz, 1H), 5.23 (s, 2H), 5.04 (q, J=8.5 Hz, 2H), 4.38 (dt, J=7.6, 5.0 Hz, 2H), 3.81 (s, 3H), 3.23-2.98 (m, 10H), 2.83 (s, 3H), 2.33 (dd, J=13.8, 9.9 Hz, 1H), 1.80 (s, 3H) ppm.

LC/MS: m/z=825.2 [M+H]⁺ amu.

### Synthesis of Compound 29

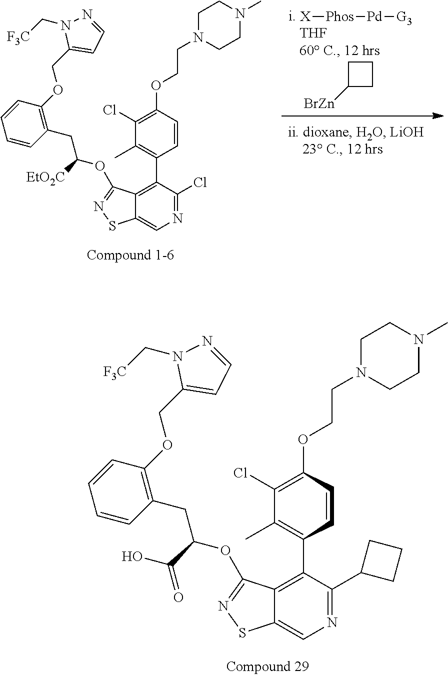

A microwave vial containing Compound 1-6 (30 mg, 0.037 mmol) was charged with X-Phos-Pd-G₃ (3 mg, 0.004 mmol). The vial was capped and after three nitrogen/vacuum cycles, the solids were dissolved in degassed THE (0.124 mL, 0.3 M). To this stirring solution was then added cyclobutylzinc bromide (0.5M in THF, 0.297 mL, 0.149 mmol). The reaction was stirred at 60° C. for 12 hours, and allowed to cool to room temperature. To this crude reaction mixture was then added dioxane (0.600 mL, 0.04M) and lithium hydroxide in water (2M solution, 0.600 mL). The reaction was allowed to stir at room temperature for 12 hours. The reaction was quenched with acetic acid (0.100 mL), diluted with DMSO and purified via reverse phase chromatography (0.25% TFA/water in 20-70% acetonitrile). The product fractions were pooled and concentrated to yield Compound 29 as an off-white amorphous solid (10.9 mg, 56% yield).

¹H NMR (500 MHz, Methanol-d₄): δ 9.25 (s, 1H), 7.56 (d, J=1.9 Hz, 1H), 7.20 (td, J=5.8, 1.0 Hz, 1H), 7.16-7.10 (m, 2H), 7.07-7.03 (m, 1H), 6.88 (td, J=6.4, 1.0 Hz, 1H), 6.55 (d, J=1.8 Hz, 1H) 6.51 (dd, J=10.0, 2.0 Hz, 1H), 5.39 (dd, J=9.7, 3.5 Hz, 1H), 5.28-5.19 (m, 2H), 5.04 (qd, J=8.8, 2.9 Hz, 2H), 4.43-4.33 (m, 2H), 3.56-3.47 (m, 1H), 3.23-3.08 (m, 6H), 2.85 (s, 3H), 2.51 (quintet, J=9 Hz, 1H), 2.42-2.28 (m, 2H), 2.12-2.03 (m, 1H), 2.01-1.89 (m, 2H), 1.88 (s, 3H), 1.87-1.79 (m, 1H) ppm (36 of 42 protons observed).

LC/MS: m/z=799.3 [M+H]⁺ amu.

### Synthesis of Compound 30

Compound 30 was synthesized with the general procedures used for Compound 29 and using cyclopropylzinc bromide. Compound 30 was obtained as an off-white amorphous solid (9.0 mg, 47% yield).

¹H NMR (500 MHz, Methanol-d₄): δ 9.01 (s, 1H), 7.56 (d, J=1.9 Hz, 1H), 7.29-7.23 (m, 1H), 7.23-7.17 (m, 1H), 7.16-7.11 (m, 1H), 7.07-7.02 (m, 1H), 6.89 (td, J=7.5, 1.0 Hz, 1H), 6.56 (d, J=1.9 Hz, 1H), 6.44 (dd, J=7.4, 1.7 Hz, 1H), 5.36 (dd, J=10.0, 3.2 Hz, 1H), 5.26-5.23 (m, 2H), 5.10-4.99 (m, 3H), 4.36 (t, J=5.0 Hz, 2H), 3.27-2.90 (m, 10H), 2.83 (s, 3H), 2.33 (dd, J=13.9, 10.0 Hz, 1H), 2.01 (s, 3H), 1.78-1.61 (m, 1H), 1.37-1.25 (m, 1H), 1.14-0.97 (m, 2H), 0.97-0.76 (m, 2H) ppm.

LC/MS: m/z=785.2 [M+H]⁺ amu.

### Synthesis of Compound 52

Compound 52 was synthesized with the general procedures used for Compound 29 and using 2-pyridylzine bromide. Compound 52 was obtained as brownish solid.

¹H NMR (400 MHz, Methanol-d₄): δ 9.50 (s, 1H), 8.85-8.73 (m, 1H), 8.22-8.08 (m, 1H), 7.83-7.74 (m, 1H), 7.44-7.34 (m, 2H), 7.26-7.14 (m, 2H), 7.11-7.03 (m, 2H), 6.83 (td, J=7.5, 0.9 Hz, 1H), 6.56 (d, J=1.9 Hz, 1H), 6.45 (dd, J=7.5, 1.7 Hz, 1H), **5.46 (dd, J=9.7, 3.8 Hz, 1H), 5.24 (s, 2H), 5.20-5.12 (m, 1H), 5.12-4.99 (m, 2H), 5.11-4.99 (m, 2H), 4.37 (t, J=4.9 Hz, 2H), 3.93-3.84 (m, 1H), 2.84 (s, 4H), 2.66 (s, 5H), 2.63 (s, 1H), 2.18 (s, 2H), 1.82 (s, 3H) ppm (39 of 39 protons observed).

LC/MS: m/z=822.2 [M+H]⁺ amu.

### Synthesis of Compound 91

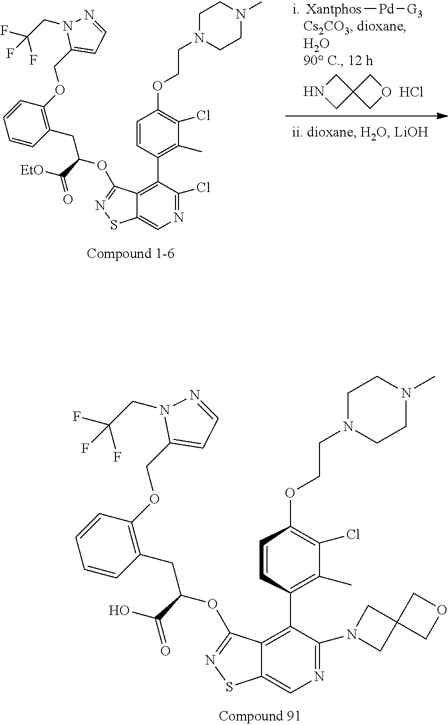

A vial was charged with Compound 1-6 (30 mg, 0.037 mmol), 2-oxa-6-azaspiro[3.3]heptane hydrochloric acid (11 mg, 0.111 mmol), XantPhos-Pd-G₃ (3.9 mg, 0.005 mmol), and cesium carbonate (12 mg, 0.037 mmol). Under nitrogen atmosphere, a solution of dioxane and water (2:1; 0.4 M) was degassed with bubbling nitrogen. The solvent was transferred into the reaction flask, which was sealed and heated to 90° C. for a period of 12 hours. After cooling, the reaction was diluted with deionized water and transferred to a separatory funnel with dichloromethane. The aqueous layer was extracted with dichloromethane and the combined organics were dried over sodium sulfate, filtered, and the volatiles were removed under reduced pressure and the resulting oil was used directly in the saponification step.

The crude product above was dissolved in a mixture of dioxane, 2N LiOH, and methanol (4:1:1, 2 mL) and left to stir at room temperature for 2 hours. After neutralizing with acetic acid, the mixture was filtered and purified by reverse phase HPLC (20 mm C18 Column, 25 mL/min, 25-60% acetonitrile.water+0.25% TFA, over 22 minutes). The fractions containing the desired product were pooled and concentrated to dryness on a lyophilizer resulting in Compound 91 as a white solid (4.6 mg product, 15% yield) that was analyzed and shown to be the anticipated product by LC/MS and proton NMR.

¹H NMR (400 MHz, Methanol-d₄): δ 8.72 (s, 1H), 7.58-7.54 (m, 1H), 7.40-7.32 (m, 1H), 7.19 (ddd, J=8.2, 7.4, 1.7 Hz, 1H), 7.13-7.00 (m, 2H), 6.84 (td, J=7.5, 1.1 Hz, 1H), 6.58-6.52 (m, 1H), 6.40-6.31 (m, 1H), 5.32 (dd, J=9.9, 3.4 Hz, 1H), 5.28-5.17 (m, 2H), 5.09-4.98 (m, 2H), 4.66 (s, 3H), 4.42-4.29 (m, 2H), 3.83-3.66 (m, 2H), 3.64-3.52 (m, 1H), 3.48-3.34 (m, 1H), 3.27-3.20 (m, 1H), 3.20-3.10 (m, 2H), 3.10-3.02 (m, 2H), 2.86-2.79 (m, 3H), 2.41-2.30 (m, 1H), 2.06-2.01 (m, 1H), 1.99 (s, 2H) ppm (35 of 43 protons observed).

LC/MS: m/z=842.3 [M+H]⁺ amu.

### Example 2: Synthesis of Compound 31

### Synthesis of Intermediate 2-1

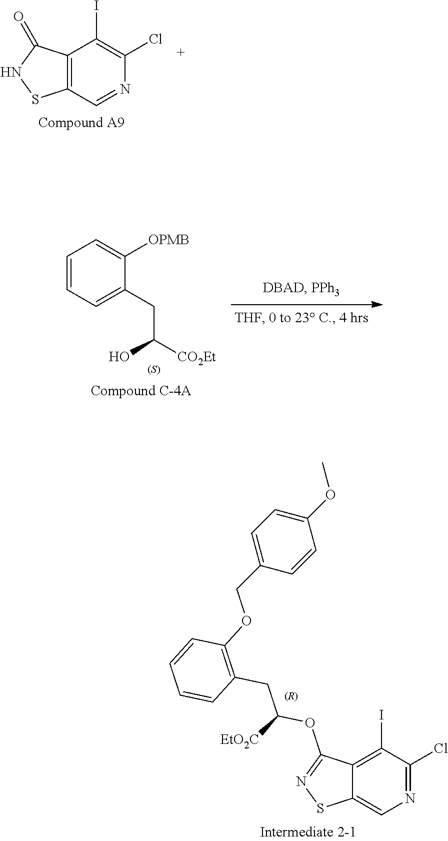

To a cooled suspension of compound A9 (108 mg, 0.344 mmol), compound C-4A (147 mg, 0.45 mmol), and triphenylphosphine (135 mg, 0.52 mmol) in tetrahydrofuran (15 mL) was added di-tert-butyl azodicarboxylate (119 mg, 0.52 mmol) in tetrahydrofuran (3.5 mL) dropwise via cannula. The reaction was removed from the cooling bath and stirred at ambient temperature for 4 hours, at which time LC/MS analysis showed complete conversion to the desired product.

The reaction was concentrated onto silica gel. Silica gel chromatography was performed with refractive index detection (0-40% ethyl acetate/hexanes). The product fractions were pooled and concentrated to yield intermediate 2-1 as a yellow oil (259 mg, quantitative yield, some impurities present).

### Synthesis of Intermediate 2-2

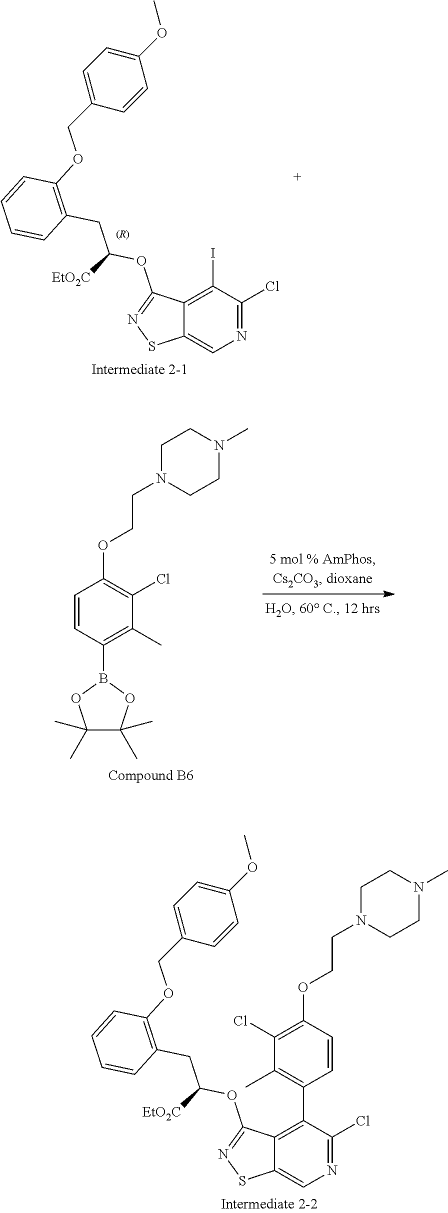

A flask containing intermediate 2-1 (257 mg, 0.344 mmol) was charged with compound B6 (250 mg, 0.633 mmol), bis(di-tert-butyl(4-dimethylaminophenyl)phosphine)dichloropalladium(II) (12.2 mg, 0.017 mmol), and cesium carbonate (336 mg, 1.03 mmol). The solids were dissolved in degassed 1,4-dioxane (1.6 mL) and deionized water (0.75 mL). The reaction was stirred at 60° C. for 12 hours, allowed to cool and poured into a separatory funnel containing water (10 mL). The organic phase was separated and the aqueous phase was washed with dichloromethane (10 mL, 3 times). The combined organic extracts were concentrated onto silica gel. Silica gel chromatography was performed (0-20% methanol/dichloromethane). The product fractions were pooled and concentrated to yield intermediate 2-2 as a mixture of diastereomers (175 mg, 56% yield).

### Synthesis of Compound 31

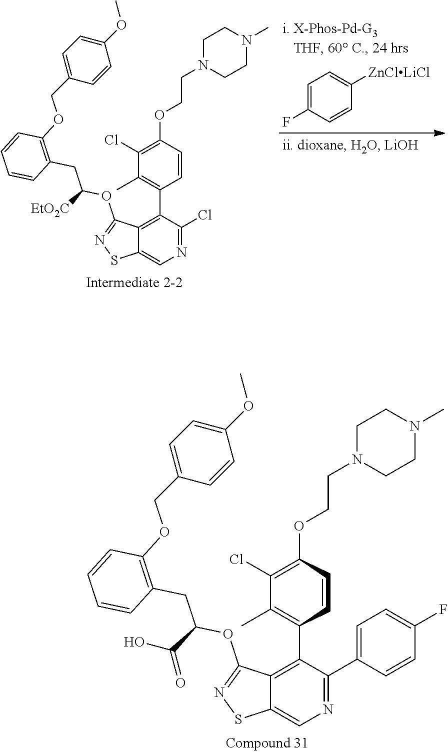

To a solution of n-BuLi (1 mL, 2.4 mmol) cooled to −78° C. was added THF (1 mL), followed by 4-fluoro-bromobenzene (0.27 mL, 2.4 mmol). The reaction was stirred for 30 minutes and a solution of zinc chloride in THF was injected (4.8 mL, 0.5 M, 2.4 mmol). The resulting Negishi reagent was allowed to warm to ambient temperature. A second reaction vessal was charged with Intermediate 2-2 (45 mg, 0.059 mmol) and X-Phos-Pd-G₃ (4.9 mg, 0.0058 mmol). After three argon/vacuum cycles, the Negishi reagent was injected (0.34 M, 3 equivalents) and the reaction was warmed to 60° C. for 24 hours. The reaction was poured into aqueous ammonium chloride and diluted with ethyl aceate. The organic phase was dried over sodium sulfate, filtered and concentrated to dryness.

The crude material was dissolved in dioxane (2 mL) and treated with aqueous lithium hydroxide (1 M, 200 μL). After 20 hours the reaction was acidified with acetic acid (20 μL), filtered and purified by reversed phase HPLC (30-70% acetonitrile/water+0.25% TFA). The active fractions were pooled, frozen and concentrated to dryness on the lyophilzer (2.8 mg).

¹H NMR (400 MHz, DMSO-d₆): δ 9.22 (s, 1H), 7.35-7.25 (m, 4H), 7.13-7.06 (m, 1H), 6.94-6.75 (m, 7H), 6.70 (td, J=7.4, 1.1 Hz, 1H), 6.54 (d, J=8.5 Hz, 1H), 5.69 (dd, J=7.6, 5.5 Hz, 1H), 4.91 (s, 2H), 4.40 (d, J=9.7 Hz, 2H), 3.76 (s, 3H), 3.53 (d, J=10.8 Hz, 2H), 3.34-3.15 (m, 3H), 3.05 (dd, J=13.9, 7.6 Hz, 1H), 2.61 (s, 3H), 1.95 (s, 3H) ppm.

LC/MS: m/z=797.2 [M+H]⁺ amu.

### Example 3: Synthesis of Compounds 3-2, 3-3, Compounds 32 Through 39, Compounds 53 Through 54, Compounds 75 Through 80, Compounds 82 Through 90, and Compound 103

### Synthesis of Compound 3-2

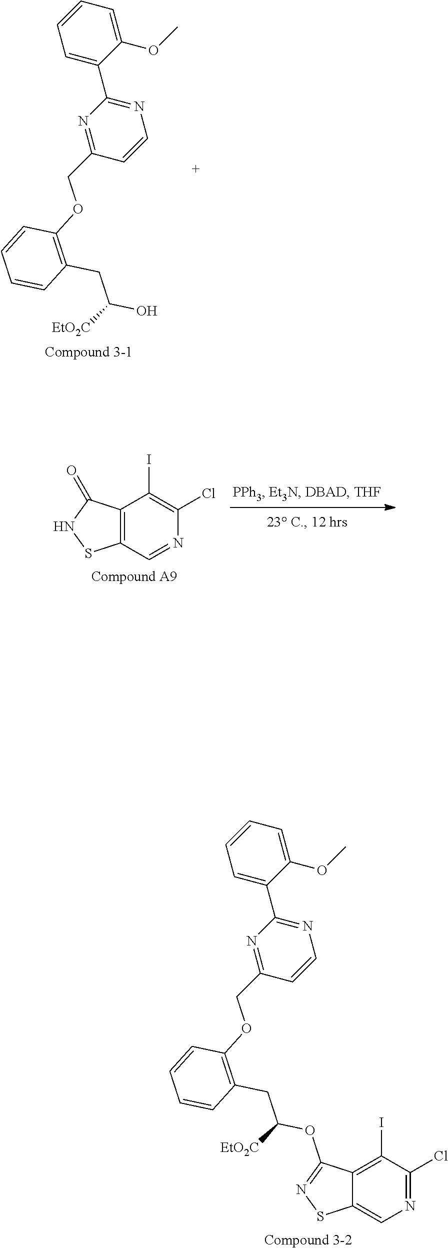 [SMILES: CCOC(=O)[C@@H](O)Cc1ccccc1OCc1ccnc(-c2ccccc2OC)n1 ; O=c1[nH]sc2cnc(Cl)c(I)c12 ; CCOC(=O)[C@@H](Cc1ccccc1OCc1ccnc(-c2ccccc2OC)n1)Oc1nsc2cnc(Cl)c(I)c12]

To a solution of Compound 3-1 (1.25 g, 3.07 mmol), PPh₃ (1.10 g, 2.88 mmol), Et₃N (1.28 mL, 9.18 mmol), and Compound A9 (0.60 g, 1.92 mmol) in THE (19.2 mL) was added DBAD in a single portion (0.755 g, 2.88 mmol). The mixture was then stirred for 12 hours. The resulting solution was adsorbed onto silica gel and purified via chromatography. The product (Compound 3-2; 1.3 g, 1.85 mmol, 96% yield) was isolated as a yellow oil.

LC/MS: m/z=675.0 [M+H]⁺ amu.

### Alternative Synthesis of Compound 3-2

To a solution of Compound 3-1 (4.0 g, 9.79 mmol), PPh₃ (3.34 g, 12.73 mmol), Et₃N (4.1 mL. 29.38 mmol), and Compound A9 (3.67 g, 11.75 mmol) in THE (48.966 mL) was added DBAD (2.93 g, 12.73 mmol) as a solution in THE (10 mL) dropwise. The mixture was then stirred for 12 hours. The resulting solution was adsorbed onto silica gel and purified via chromatography. The product (Compound 3-2; 3.5 g, 4.979 mmol, 50.84% yield) was isolated as a yellow oil.

LC/MS: m/z=675.0 [M+H]⁺ amu.

### Synthesis of Compound 3-3

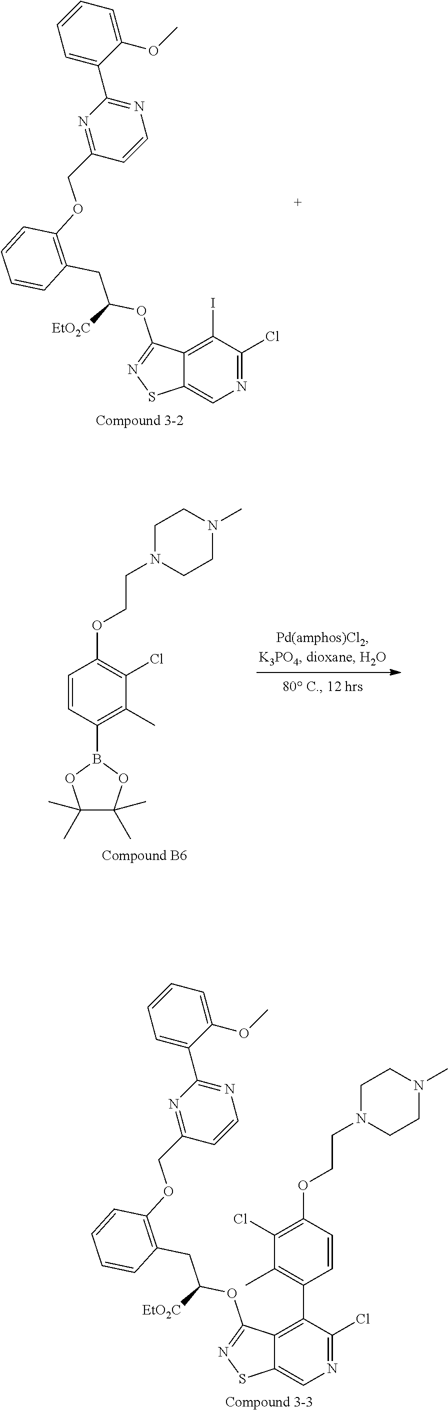

A round bottom flask containing Compound 3-2 (3.5 g, 4.98 mmol), Pd(amphos)C₁₂ (176.28 mg, 0.2500 mmol), Compound B6 (2.36 g, 5.97 mmol), and K₃PO₄ (3.17 g, 14.94 mmol) was evacuated and back filled with nitrogen three times, and then freshly degassed dioxane (16 mL) and water (8 mL) were added. The mixture was then heated to 80° C. for 12 hours. The solution was cooled to ambient temperature, diluted with ethyl acetate, partitioned with saturated ammonium chloride and extracted with ethyl acetate three times. The combined organic washings were dried over MgSO₄, concentrated, and purified via flash chromatography (0-25% DCM/MeOH). Compound 3-3 (3.78 g, 4.4796 mmol, 89.97% yield) was isolated as a light-yellow solid.

LC/MS: m/z=815.1 [M+H]⁺ amu.

### Synthesis of Compound 32

Compound 32 was synthesized using Compound 3-3, the corresponding heteroaryl boronate ester or acid, and following the general procedures used to synthesize Compound 7. Compound 32 was obtained as an off-white solid.

¹H NMR (300 MHz, Methanol-d₄): δ 9.35 (s, 1H), 8.89 (d, J=5.5 Hz, 1H), 7.96 (dd, J=18.6, 5.3 Hz, 2H), 7.81 (d, J=7.6 Hz, 1H), 7.61-7.50 (m, 1H), 7.41 (d, J=8.5 Hz, 1H), 7.30-7.08 (m, 4H), 7.00 (d, J=7.9 Hz, 1H), 6.96-6.80 (m, 3H), 6.43 (d, J=7.5 Hz, 1H), 5.57 (td, J=10.2, 2.8 Hz, 1H), 5.33 (d, J=5.6 Hz, 2H), 4.44-4.32 (m, 2H), 4.30-4.12 (m, 2H), 3.92 (d, J=1.2 Hz, 3H), 3.17 (s, 8H), 2.85 (d, J=4.9 Hz, 3H), 1.80 (s, 2H), 1.35 (s, 3H) ppm.

LC/MS: m/z=902.3 [M+H]⁺ amu.

### Synthesis of Compound 33

Compound 33 was synthesized using Compound 3-3, the corresponding heteroaryl boronate ester or acid, and following the general procedures used to synthesize Compound 7. Compound 33 was obtained as an off-white solid.

¹H NMR (300 MHz, Methanol-d₄): δ 9.34 (d, J=3.1 Hz, 1H), 8.93 (d, J=5.6 Hz, 1H), 8.08-7.82 (m, 2H), 7.59 (t, J=7.3 Hz, 1H), 7.43 (d, J=8.6 Hz, 1H), 7.31-7.08 (m, 4H), 7.03-6.75 (m, 4H), 6.42 (d, J=7.3 Hz, 1H), 5.67-5.49 (m, 1H), 5.38 (d, J=5.8 Hz, 2H), 4.32 (m, 2H), 3.97 (d, J=1.5 Hz, 3H), 3.88 (d, J=1.9 Hz, 3H), 3.43 (d, J=21.4 Hz, 8H), 2.65 (s, 3H), 2.58-2.43 (m, 1H), 1.80 (s, 3H) ppm.

LC/MS: m/z=888.3 [M+H]⁺ amu.

### Synthesis of Compound 34

Compound 34 was synthesized using Compound 3-3, the corresponding aryl boronate ester or acid, and following the general procedures used to synthesize Compound 7. Compound 34 was obtained as an off-white amorphous solid (4.2 mg, 17% yield).

¹H NMR (400 MHz, Methanol-d₄): δ 9.30 (s, 1H), 8.88 (d, J=5.4 Hz, 1H), 7.90 (d, J=5.4 Hz, 1H), 7.75 (dd, J=7.7, 1.8 Hz, 1H), 7.59-7.49 (m, 2H), 7.41 (d, J=8.5 Hz, 1H), 7.26-7.06 (m, 6H), 7.02-6.95 (m, 1H), 6.93-6.86 (m, 2H), 6.83 (td, J=7.4, 1.0 Hz, 1H), 6.39 (dd, J=7.5, 1.7 Hz, 1H), 6.33 (d, J=2.2 Hz, 1H), 5.58 (dd, J=10.2, 3.1 Hz, 1H), 5.38-5.24 (m, 2H), 5.02 (s, 2H), 4.42-4.28 (m, 2H), 3.89 (d, J=3.9 Hz, 6H), 3.44 (dd, J=13.9, 3.1 Hz, 1H), 3.29-3.17 (m, 4H), 3.17-2.94 (m, 5H), 2.83 (s, 3H), 2.66 (s, 2H), 2.50 (dd, J=14.0, 10.2 Hz, 1H), 1.73 (s, 3H) ppm (51 of 51 protons observed).

LC/MS: m/z=967.4 [M+H]⁺ amu.

### Synthesis of Compound 35

Compound 35 was synthesized using Compound 3-3, the corresponding aryl boronate ester or acid, and following the general procedures used to synthesize Compound 7. Compound 35 was obtained as an off-white amorphous solid (56 mg, 54% yield).

¹H NMR (400 MHz, Methanol-d₄): δ 9.30 (s, 1H), 8.85 (d, J=5.3 Hz, 1H), 7.83 (d, J=5.3 Hz, 1H), 7.62 (dd, J=7.5, 1.8 Hz, 1H), 7.52-7.45 (ddd, J=8.9, 7.4, 1.8 Hz, 1H), 7.43 (d, J=8.5 Hz, 1H), 7.29-7.20 (m, 1H), 7.20-7.04 (m, 5H), 7.04-6.92 (m, 3H), 6.81 (t, J=7.4 Hz, 1H), 6.42 (dd, J=7.4, 1.6 Hz, 1H), 5.56 (dd, J=10.1, 3.1 Hz, 1H), 5.33-5.19 (m, 2H), 4.38-4.21 (m, 2H), 3.84 (s, 3H), 3.50-3.37 (m, 1H), 2.99 (t, J=4.9 Hz, 2H), 2.79 (s, 3H), 2.52 (dd, J=14.0, 10.1 Hz, 1H), 1.73 (s, 3H) ppm (35 of 44 protons observed).

LC/MS: m/z=875.3 [M+H]⁺ amu.

### Synthesis of Compound 36

Compound 36 was synthesized using Compound 3-3, cyclobutylzine bromide, and following the general procedures used to synthesize Compound 29. Compound 36 was obtained as an off-white solid.

¹H NMR (400 MHz, Methanol-d₄): δ 9.07 (s, 1H), 8.75 (d, J=5.2 Hz, 1H), 7.82 (d, J=5.2 Hz, 1H), 7.52 (dd, J=7.5, 1.8 Hz, 1H), 7.43-7.27 (m, 2H), 7.17 (d, J=8.5 Hz, 1H), 7.09-6.95 (m, 5H), 6.81 (d, J=8.3 Hz, 1H), 6.77-6.70 (m, 1H), 6.46 (dd, J=7.5, 1.7 Hz, 1H), 5.35 (dd, J=10.2, 2.9 Hz, 1H), 5.22-5.10 (m, 2H), 4.19 (t, J=5.1 Hz, 2H), 3.74 (s, 3H), 3.44-3.35 (m, 2H), 2.83-2.70 (m, 10H), 2.50-2.35 (m, 5H), 2.26 (q, J=9.2 Hz, 1H), 1.71 (s, 5H) ppm.

LC/MS: m/z=835.3 [M+H]⁺ amu.

### Synthesis of Compound 103

Compound 103 was synthesized using Compound 3-3, cyclobutylzinc bromide, and performing the first step, but not the second step, used to synthesize Compound 29. Compound 103 was obtained as an off-white solid.

¹H NMR (400 MHz, Acetonitrile-d₃): δ 9.57-9.42 (m, 1H), 9.00 (d, J=5.5 Hz, 1H), 8.43-8.28 (m, 1H), 7.99 (t, J=5.2 Hz, 1H), 7.73 (ddq, J=8.6, 7.3, 1.5 Hz, 1H), 7.36-7.30 (m, 1H), 7.28-7.18 (m, 2H), 7.17-7.08 (m, 2H), 7.00-6.91 (m, 2H), 6.71 (dd, J=7.4, 1.7 Hz, 1H), 5.50 (dd, J=9.0, 4.6 Hz, 1H), 5.45 (d, J=2.0 Hz, 2H), 4.69 (t, J=4.7 Hz, 2H), 4.14 (d, J=3.4 Hz, 2H), 4.05 (qd, J=7.1, 1.3 Hz, 2H), 3.94-3.76 (m, 3H), 3.71-3.44 (m, 5H), 3.23 (dd, J=14.2, 4.6 Hz, 1H), 2.76 (s, 3H), 2.74-2.53 (m, 2H), 2.22-2.02 (m, 1H), 1.97 (d, J=1.9 Hz, 4H), 1.06 (t, J=7.1 Hz, 3H) ppm.

LC/MS: m/z=862.3 [M+H]⁺ amu.

### Synthesis of Compound 37

Compound 37 was synthesized using Compound 3-3, the corresponding aryl boronate ester or acid, and following the general procedures used to synthesize Compound 7. Compound 37 was obtained as an off-white solid.

¹H NMR (300 MHz, Methanol-d₄): δ 9.30 (s, 1H), 8.90 (d, J=5.5 Hz, 1H), 7.95 (d, J=5.6 Hz, 1H), 7.83 (dd, J=7.7, 1.8 Hz, 1H), 7.60-7.50 (m, 1H), 7.43 (d, J=8.5 Hz, 1H), 7.26-7.06 (m, 3H), 7.03-6.88 (m, 4H), 6.83 (t, J=7.4 Hz, 1H), 6.39 (d, J=6.8 Hz, 1H), 5.58 (dd, J=10.2, 2.9 Hz, 1H), 5.40-5.24 (m, 2H), 4.38 (t, J=4.8 Hz, 2H), 3.92 (s, 3H), 3.23 (q, J=6.3, 5.0 Hz, 8H), 2.86 (d, J=3.7 Hz, 3H), 2.62-2.38 (m, 1H), 1.76 (s, 3H) ppm.

LC/MS: m/z=905.3 [M+H]⁺ amu.

### Synthesis of Compound 38

Compound 38 was synthesized using Compound 3-3, the corresponding heteroaryl boronate ester or acid, and following the general procedures used to synthesize Compound 7. Compound 38 was obtained as an off-white solid.

¹H NMR (300 MHz, Methanol-d₄): δ 9.21 (s, 1H), 8.89 (d, J=5.6 Hz, 1H), 7.94 (d, J=5.5 Hz, 1H), 7.80 (d, J=7.8 Hz, 1H), 7.55 (t, J=8.4 Hz, 1H), 7.41-7.31 (m, 2H), 7.24 (dd, J=14.1, 8.4 Hz, 2H), 7.11 (d, J=7.9 Hz, 2H), 7.00 (d, J=8.1 Hz, 1H), 6.89 (t, J=7.3 Hz, 1H), 6.46 (d, J=7.3 Hz, 1H), 5.58 (d, J=8.5 Hz, 1H), 5.44-5.25 (m, 2H), 4.42 (s, 2H), 3.91 (s, 3H), 3.83 (d, J=8.5 Hz, 1H), 3.46 (d, J=13.8 Hz, 1H), 3.19 (s, 7H), 2.85 (s, 3H), 2.45 (dd, J=14.1, 10.4 Hz, 1H), 2.04 (dd, J=12.9, 5.9 Hz, 1H), 1.81 (s, 3H), 0.83 (t, J=6.5 Hz, 5H) ppm.

LC/MS: m/z=903.3 [M+H]⁺ amu.

### Synthesis of Compound 39

Compound 39 was synthesized using Compound 3-3, the corresponding aryl boronate ester or acid, and following the general procedures used to synthesize

Compound 7. Compound 39 was obtained as an off-white solid.

¹H NMR (500 MHz, Methanol-d₄): δ 9.38 (s, 1H), 8.97 (d, J=5.6 Hz, 1H), 8.05 (d, J=5.7 Hz, 1H), 7.94 (dt, J=5.9, 1.7 Hz, 1H), 7.63 (ddd, J=9.0, 7.4, 1.8 Hz, 1H), 7.48 (d, J=8.6 Hz, 1H), 7.32-7.12 (m, 5H), 7.05 (d, J=8.1 Hz, 1H), 6.98-6.81 (m, 4H), 6.45 (dd, J=7.5, 1.7 Hz, 1H), 5.64 (dd, J=10.2, 3.0 Hz, 1H), 5.42 (q, J=15.7 Hz, 2H), 4.46 (t, J=4.9 Hz, 2H), 4.01 (s, 3H), 3.71 (s, 3H), 3.56-3.40 (m, 7H), 2.93 (s, 3H), 2.56 (dd, J=14.0, 10.2 Hz, 1H) ppm.

LC/MS: m/z=887.3 [M+H]⁺ amu.

### Synthesis of Compound 40

Compound 40 was synthesized using Compound 3-3, the corresponding aryl boronate ester or acid, and following the general procedures used to synthesize Compound 7. Compound 40 was obtained as an off-white solid.

¹H NMR (500 MHz, Methanol-d₄): δ 9.38 (d, J=2.2 Hz, 1H), 9.00 (d, J=5.8 Hz, 1H), 8.11 (d, J=5.7 Hz, 1H), 8.04 (dq, J=6.1, 1.9 Hz, 1H), 7.73-7.59 (m, 2H), 7.50 (d, J=8.5 Hz, 1H), 7.41-7.29 (m, 3H), 7.28-7.15 (m, 3H), 7.05 (td, J=8.6, 2.3 Hz, 3H), 6.89 (t, J=7.4 Hz, 1H), 6.42 (dd, J=7.5, 1.7 Hz, 1H), 5.64 (dd, J=10.3, 2.8 Hz, 1H), 5.45 (q, J=16.0 Hz, 2H), 4.52 (t, J=4.9 Hz, 2H), 4.05 (d, J=3.0 Hz, 3H), 3.62-3.42 (m, 7H), 2.97 (d, J=3.1 Hz, 3H), 2.55 (dd, J=14.0, 10.3 Hz, 1H), 1.81 (s, 3H) ppm.

LC/MS: m/z=876.4 [M+H]⁺ amu.

### Synthesis of Compound 53

Compound 53 was synthesized using Compound 3-3, the corresponding heteroaryl boronate ester or acid, and following the general procedure used to synthesize Compound 7. Compound 53 was obtained as a brown solid.

¹H NMR (400 MHz, Methanol-d₄): δ 9.44 (s, 1H), 8.86 (d, J=5.3 Hz, 1H), 7.82 (d, J=5.3 Hz, 1H), 7.80-7.75 (m, 1H), 7.66 (dd, J=7.6, 1.7 Hz, 1H), 7.51 (ddd, J=8.4, 7.4, 1.8 Hz, 1H), 7.46 (d, J=8.5 Hz, 1H), 7.23-7.14 (m, 3H), 7.08 (td, J=7.5, 1.0 Hz, 1H), 6.99 (d, J=8.1 Hz, 1H), 6.83 (t, J=7.3 Hz, 1H), 6.44 (dd, J=6.8, 2H), 5.61 (dd, J=10.0, 3.2, 1H), 5.35-5.22 (m, 3H), 4.33 (t, J=4.9 Hz, 2H), 3.93-3.87 (m, 2H), 3.86 (s, 3H), 3.09-3.01 (m, 5H), 2.81 (s, 3H), 2.66 (s, 1H), 2.61-2.39 (m, 2H), 2.38-2.24 (m, 1H), 2.07-1.90 (m, 3H), 1.78 (s, 3H) ppm (44 of 44 protons observed).

LC/MS: m/z=858.3 [M+H]⁺ amu.

### Synthesis of Compound 54

Compound 54 was synthesized using Compound 3-3, the corresponding heterocyclic boronate ester or acid, and following the general procedure used to synthesize Compound 7. Compound 54 was obtained as an off-white oil.

¹H NMR (400 MHz, Methanol-d₄): δ 9.22 (s, 1H), 8.86 (d, J=5.4 Hz, 1H), 7.86 (d, J=5.4 Hz, 1H), 7.72 (dd, J=7.7, 1.8 Hz, 1H), 7.52 (ddd, J=9.0, 7.4, 1.8 Hz, 1H), 7.30 (d, J=8.5 Hz, 1H), 7.25-7.17 (m, 2H), 7.17-7.03 (m, 2H), 7.03-6.95 (m, 1H), 6.89 (td, J=7.5, 1.0 Hz, 1H), 6.51 (dd, J=7.5, 1.7 Hz, 1H), 5.74-7.68 (m, 1H), 5.57 (dd, J=9.9, 3.4 Hz, 1H), 5.37-3.24 (m, 2H), 4.42-4.28 (m, 2H), 4.14-3.98 (m, 2H), 3.88 (s, 3H), 3.71 (t, J=5.6 Hz, 2H), 3.38 (dd, J=14.0, 3.4 Hz, 1H), 3.27 (s, 3H), 3.10 (t, J=4.9 Hz, 3H), 2.83 (s, 3H), 2.53 (dd, J=14.0, 9.9 Hz, 1H), 2.36-2.26 (m, 1H), 2.23-2.07 (m, 1H), 1.96 (s, 3H), 1.36-1.20 (m, 5H) ppm (47 of 47 protons observed).

LC/MS: m/z=863.2 [M+H]⁺ amu.

### Synthesis of Compound 75

Compound 75 was synthesized using Compound 3-3, the corresponding heteroaryl boronate ester or acid, and following the general procedures used to synthesize Compound 7. Compound 75 was obtained as an amorphous off-white solid.

¹H NMR (300 MHz, Methanol-d₄): δ 9.22 (s, 1H), 8.93 (dd, J=5.7, 3.5 Hz, 1H), 8.06 (d, J=5.8 Hz, 1H), 8.03-7.91 (m, 1H), 7.65-7.51 (m, 2H), 7.38 (d, J=8.5 Hz, 1H), 7.32-7.08 (m, 4H), 7.02 (d, J=8.2 Hz, 1H), 6.97-6.83 (m, 2H), 6.48 (dd, J=7.4, 1.7 Hz, 1H), 5.59 (dd, J=10.3, 2.7 Hz, 1H), 5.49-5.28 (m, 2H), 4.52 (t, J=4.9 Hz, 2H), 4.38-4.27 (m, 1H), 4.21-4.05 (m, 3H), 3.98 (s, 2H), 3.44 (s, 11H), 2.91 (s, 3H), 2.46 (dd, J=14.0, 10.3 Hz, 1H), 2.33 (t, J=7.3 Hz, 1H), 2.06-2.02 (m, 2H), 1.83 (s, 2H) ppm.

LC/MS: m/z=875.3 [M+H]⁺ amu.

### Synthesis of Compound 76

Compound 76 was synthesized using Compound 3-3, the corresponding heteroaryl boronate ester or acid, and following the general procedures used to synthesize Compound 7. Compound 76 was obtained as an amorphous off-white solid.

¹H NMR (300 MHz, Methanol-d₄): δ 9.43 (s, 1H), 8.93 (d, J=5.7 Hz, 1H), 8.01 (d, J=5.7 Hz, 1H), 7.91 (dd, J=7.8, 1.8 Hz, 1H), 7.63-7.48 (m, 2H), 7.38 (d, J=8.5 Hz, 1H), 7.27-7.16 (m, 3H), 7.11 (td, J=7.6, 1.0 Hz, 1H), 7.01 (d, J=8.2 Hz, 1H), 6.89 (dd, J=7.9, 6.9 Hz, 1H), 6.50 (dd, J=7.4, 1.7 Hz, 1H), 5.63 (dd, J=10.3, 2.8 Hz, 1H), 5.54 (d, J=2.4 Hz, 1H), 5.46-5.29 (m, 2H), 4.51 (t, J=4.8 Hz, 2H), 4.21 (q, J=7.3 Hz, 2H), 3.95 (s, 3H), 3.56-3.37 (m, 10H), 2.90 (s, 3H), 2.47 (dd, J=14.1, 10.4 Hz, 1H), 1.86 (d, J=3.1 Hz, 4H), 1.44 (t, J=7.3 Hz, 3H) ppm.

LC/MS: m/z=875.3 [M+H]⁺ amu.

### Synthesis of Compound 77

Compound 77 was synthesized using Compound 3-3, the corresponding heteroaryl boronate ester or acid, and following the general procedures used to synthesize Compound 7. Compound 77 was obtained as an amorphous off-white solid.

¹H NMR (300 MHz, Methanol-d₄): δ 9.29 (s, 1H), 8.93 (dd, J=5.8, 3.2 Hz, 1H), 8.04 (d, J=5.8 Hz, 1H), 7.96 (dd, J=7.8, 1.8 Hz, 1H), 7.60 (ddd, J=8.4, 7.4, 1.8 Hz, 1H), 7.33-7.18 (m, 5H), 7.16-7.08 (m, 1H), 7.03 (d, J=8.0 Hz, 1H), 6.97-6.86 (m, 1H), 6.54 (dd, J=7.3, 1.7 Hz, 1H), 6.08 (s, 1H), 5.62 (dd, J=10.2, 2.8 Hz, 1H), 5.37 (dd, J=16.2, 5.5 Hz, 3H), 4.55-4.39 (m, 3H), 3.97 (d, J=1.0 Hz, 5H), 3.41 (t, J=7.0 Hz, 20H), 2.90 (d, J=1.6 Hz, 5H), 2.44 (s, 3H), 1.89 (d, J=5.4 Hz, 3H) ppm.

LC/MS: m/z=862.3 [M+H]⁺ amu.

### Synthesis of Compound 78

Compound 78 was synthesized using Compound 3-3, the corresponding heteroaryl boronate ester or acid, and following the general procedures used to synthesize Compound 7. Compound 78 was obtained as an amorphous off-white solid.

¹H NMR (300 MHz, Methanol-d₄): δ 9.27 (s, 1H), 8.90 (d, J=5.5 Hz, 1H), 7.96 (d, J=5.7 Hz, 1H), 7.83 (dd, J=7.7, 1.8 Hz, 1H), 7.62-7.52 (m, 1H), 7.31-7.17 (m, 5H), 7.10 (t, J=7.6 Hz, 1H), 7.02 (dd, J=8.3, 3.9 Hz, 1H), 6.95-6.88 (m, 1H), 6.57 (d, J=7.7 Hz, 1H), 5.61 (dd, J=10.3, 3.0 Hz, 1H), 5.49 (s, 1H), 5.39-5.30 (m, 2H), 4.46-4.36 (m, 2H), 3.92 (s, 4H), 3.25-2.97 (m, 3H), 2.85 (s, 4H), 2.45 (dd, J=14.1, 10.2 Hz, 1H), 2.06-1.99 (m, 3H), 1.88 (s, 3H) ppm.

LC/MS: m/z=888.3 [M+H]⁺ amu.

### Synthesis of Compound 79

Compound 79 was synthesized using Compound 3-3, the corresponding heteroaryl boronate ester or acid, and following the general procedures used to synthesize Compound 7. Compound 79 was obtained as an amorphous off-white solid.

LC/MS: m/z=903.3 [M+H]⁺ amu.

### Synthesis of Compound 80

Compound 80 was synthesized using Compound 3-3, the corresponding aryl boronate ester or acid, and following the general procedures used to synthesize Compound 7. Compound 80 was obtained as an amorphous off-white solid.

¹H NMR (300 MHz, Methanol-d₄): δ 9.34 (d, J=0.4 Hz, 1H), 8.91 (d, J=5.6 Hz, 1H), 7.98 (d, J=5.6 Hz, 1H), 7.88 (dd, J=7.7, 1.7 Hz, 1H), 7.78-7.54 (m, 3H), 7.54-7.41 (m, 4H), 7.30-7.05 (m, 4H), 6.99 (d, J=8.2 Hz, 1H), 6.34 (d, J=7.5 Hz, 1H), 5.58 (dd, J=10.4, 2.9 Hz, 1H), 5.42-5.25 (m, 2H), 4.39 (d, J=5.2 Hz, 2H), 3.95 (d, J=1.4 Hz, 3H), 3.53-3.42 (m, 1H), 3.19 (s, 9H), 2.86 (d, J=1.7 Hz, 3H), 2.50 (dd, J=13.9, 10.4 Hz, 1H), 1.69 (s, 3H) ppm.

LC/MS: m/z=882.3 [M+H]⁺ amu.

### Synthesis of Compound 82

Compound 82 was synthesized using Compound 3-3, the corresponding aryl boronate ester or acid, and following the general procedures used to synthesize Compound 7. Compound 82 was obtained as an amorphous off-white solid.

¹H NMR (300 MHz, Methanol-d₄): δ 9.31 (s, 1H), 8.88 (d, J=5.4 Hz, 1H), 7.90 (d, J=5.4 Hz, 1H), 7.77 (d, J=7.5 Hz, 1H), 7.54 (t, J=7.8 Hz, 1H), 7.30 (d, J=8.3 Hz, 2H), 7.21 (q, J=7.1 Hz, 2H), 7.14-6.91 (m, 4H), 6.85 (t, J=7.5 Hz, 1H), 6.45 (d, J=7.4 Hz, 1H), 5.64-5.55 (m, 1H), 5.41-5.25 (m, 2H), 4.28 (s, 2H), 3.90 (s, 3H), 3.09 (s, 4H), 2.82 (s, 3H), 2.53 (dd, J=14.0, 9.8 Hz, 1H), 1.83 (s, 3H) ppm.

LC/MS: m/z=875.3 [M+H]⁺ amu.

### Synthesis of Compound 83

Compound 83 was synthesized using Compound 3-3, the corresponding aryl boronate ester or acid, and following the general procedures used to synthesize Compound 7. Compound 83 was obtained as an amorphous off-white solid.

¹H NMR (300 MHz, Methanol-d₄): δ 9.33 (s, 1H), 8.90 (d, J=5.5 Hz, 1H), 7.94 (d, J=5.6 Hz, 1H), 7.83 (dd, J=7.7, 1.7 Hz, 1H), 7.61-7.46 (m, 1H), 7.31-7.15 (m, 5H), 7.11 (td, J=7.5, 1.0 Hz, 1H), 7.06-6.92 (m, 5H), 6.87 (t, J=7.4 Hz, 1H), 6.50 (d, J=7.4 Hz, 1H), 5.61 (dd, J=9.8, 3.3 Hz, 1H), 5.43-5.24 (m, 2H), 4.29 (t, J=4.8 Hz, 2H), 3.31 (s, 3H), 3.17 (s, 6H), 2.84 (s, 3H), 2.52 (dd, J=14.0, 9.9 Hz, 1H), 2.13 (s, 3H), 1.88 (s, 3H) ppm.

LC/MS: m/z=871.3 [M+H]⁺ amu.

### Synthesis of Compound 84

Compound 84 was synthesized using Compound 3-3, the corresponding aryl boronate ester or acid, and following the general procedures used to synthesize Compound 7. Compound 84 was obtained as an amorphous off-white solid.

¹H NMR (300 MHz, Methanol-d₄): δ 9.31 (s, 1H), 8.88 (d, J=5.5 Hz, 1H), 7.90 (d, J=5.4 Hz, 1H), 7.75 (dd, J=7.7, 1.8 Hz, 1H), 7.58-7.48 (m, 1H), 7.44 (d, J=8.5 Hz, 1H), 7.24-7.13 (m, 3H), 7.13-6.94 (m, 4H), 6.83 (t, J=7.7 Hz, 1H), 6.40 (d, J=5.9 Hz, 1H), 5.58 (dd, J=10.3, 3.0 Hz, 1H), 5.37-5.15 (m, 2H), 3.89 (s, 3H), 3.54-3.32 (m, 1H), 3.10 (t, J=4.9 Hz, 3H), 2.83 (s, 3H), 1.76 (s, 3H) ppm.

LC/MS: m/z=911.3 [M+H]⁺ amu.

### Synthesis of Compound 85

Compound 85 was synthesized using Compound 3-3, the corresponding aryl boronate ester or acid, and following the general procedures used to synthesize Compound 7. Compound 85 was obtained as an amorphous off-white solid.

¹H NMR (300 MHz, Methanol-d₄): δ 9.30 (s, 1H), 8.90 (d, J=5.5 Hz, 1H), 7.95 (d, J=5.5 Hz, 1H), 7.82 (d, J=7.6 Hz, 1H), 7.56 (t, J=7.9 Hz, 1H), 7.43 (d, J=8.5 Hz, 1H), 7.29-7.04 (m, 5H), 6.99 (d, J=8.2 Hz, 1H), 6.83 (t, J=7.6 Hz, 1H), 6.38 (d, J=7.4 Hz, 1H), 5.57 (dd, J=10.2, 2.8 Hz, 1H), 5.42-5.25 (m, 2H), 4.37 (s, 2H), 3.92 (s, 3H), 3.64-3.33 (m, 1H), 3.11 (d, J=26.2 Hz, 4H), 2.84 (s, 3H), 2.50 (dd, J=13.9, 10.3 Hz, 1H), 1.75 (s, 3H) ppm.

LC/MS: m/z=893.3 [M+H]⁺ amu.

### Synthesis of Compound 86

Compound 86 was synthesized using Compound 3-3, the corresponding aryl boronate ester or acid, and following the general procedures used to synthesize Compound 7. Compound 86 was obtained as an amorphous off-white solid.

¹H NMR (300 MHz, Methanol-d₄): δ 9.31 (d, J=0.8 Hz, 1H), 8.89 (d, J=5.5 Hz, 1H), 7.92 (d, J=5.4 Hz, 1H), 7.83-7.75 (m, 1H), 7.72-7.46 (m, 2H), 7.30-7.14 (m, 2H), 7.10 (t, J=7.5 Hz, 1H), 7.06-6.83 (m, 5H), 6.75 (d, J=8.5 Hz, 1H), 6.49 (d, J=7.7 Hz, 1H), 5.60 (dd, J=9.8, 3.4 Hz, 1H), 5.41-5.23 (m, 2H), 4.31 (d, J=5.3 Hz, 2H), 3.91 (d, J=1.7 Hz, 3H), 3.24 (s, OH), 3.20-3.15 (m, 7H), 2.85 (s, 3H), 2.14 (s, 3H), 1.87 (s, 3H) ppm.

LC/MS: m/z=889.3 [M+H]⁺ amu.

### Synthesis of Compound 87

Compound 87 was synthesized using Compound 3-3, the corresponding aryl boronate ester or acid, and following the general procedures used to synthesize Compound 7. Compound 87 was obtained as an amorphous off-white solid.

¹H NMR (300 MHz, Methanol-d₄): δ 9.34 (s, 1H), 8.93 (d, J=5.7 Hz, 1H), 8.03 (d, J=5.7 Hz, 1H), 7.95 (dd, J=7.8, 1.7 Hz, 1H), 7.69-7.51 (m, 1H), 7.42 (d, J=8.6 Hz, 1H), 7.28-7.07 (m, 5H), 7.04-6.96 (m, 1H), 6.90-6.79 (m, 4H), 6.40 (dd, J=7.4, 1.7 Hz, 1H), 5.59 (dd, J=10.2, 2.8 Hz, 1H), 5.48-5.28 (m, 2H), 4.42 (t, J=4.8 Hz, 2H), 3.97 (s, 3H), 3.92-3.72 (m, 2H), 3.50-3.33 (m, 12H), 2.88 (s, 3H), 2.49 (dd, J=14.0, 10.3 Hz, 1H), 1.78 (s, 3H), 1.32 (t, J=7.0 Hz, 3H) ppm.

LC/MS: m/z=901.3 [M+H]⁺ amu.

### Synthesis of Compound 88

Compound 88 was synthesized using Compound 3-3, the corresponding aryl boronate ester or acid, and following the general procedures used to synthesize Compound 7. Compound 88 was obtained as an off-white solid.

¹H NMR (400 MHz, Methanol-d₄): δ 9.30 (s, 1H), 8.84 (d, J=5.3 Hz, 1H), 7.81 (d, J=5.3 Hz, 1H), 7.64 (dd, J=7.7, 1.8 Hz, 1H), 7.54-7.45 (m, 1H), 7.44-7.36 (m, 2H), 7.29-7.04 (m, 6H), 6.98 (d, J=8.2 Hz, 1H), 6.82 (t, J=7.5 Hz, 1H), 6.41 (d, J=7.1 Hz, 1H), 5.58 (dd, J=10.1, 3.1 Hz, 1H), 5.35-5.18 (m, 2H), 4.37-4.25 (m, 2H), 3.85 (s, 3H), 3.50-3.46 (m, 1H), 3.41 (dd, J=14.4, 2.8 Hz, 1H), 3.15-3.10 (m, 1H), 3.00 (t, J=4.9 Hz, 2H), 2.80 (s, 3H), 2.52 (dd, J=14.0, 10.1 Hz, 1H), 1.99 (s, 1H), 1.73 (s, 3H), 1.29 (s, 1H) ppm (38 of 43 protons observed).

LC/MS: m/z=909.3 [M+H]⁺ amu.

### Synthesis of Compound 89

Compound 89 was synthesized using Compound 3-3, the corresponding heteroaryl boronate ester or acid, and following the general procedures used to synthesize Compound 7. Compound 89 was obtained as an off-white solid.

¹H NMR (400 MHz, Methanol-d₄): δ 9.21 (s, 1H), 8.87 (d, J=5.4 Hz, 1H), 7.90 (d, J=5.4 Hz, 1H), 7.74 (dd, J=7.6, 1.8 Hz, 1H), 7.58-7.48 (m, 1H), 7.40-7.31 (m, 2H), 7.30-7.24 (m, 1H), 7.25-7.16 (m, 3H), 7.10 (td, J=7.5, 1.0 Hz, 1H), 7.04-6.97 (m, 1H), 6.90 (td, J=7.4, 1.0 Hz, 1H), 6.51 (dd, J=7.5, 1.7 Hz, 1H), 5.60 (dd, J=10.3, 2.9 Hz, 1H), 5.38-5.21 (m, 2H), 4.40 (t, J=4.9 Hz, 2H), 4.02-3.91 (m, 1H), 3.87-3.80 (m, 1H), 3.45 (dd, J=13.9, 3.0 Hz, 1H), 3.22-2.97 (m, 5H), 2.84 (s, 3H), 2.48 (dd, J=14.0, 10.3 Hz, 1H), 1.82 (s, 3H), 1.23-1.08 (m, 1H), 0.65-0.52 (m, 2H), 0.35-0.23 (m, 2H) ppm (40 of 49 protons observed).

LC/MS: m/z=901.3 [M+H]⁺ amu.

### Synthesis of Compound 90

Compound 90 was synthesized using Compound 3-3, the corresponding heteroaryl boronate ester or acid, and following the general procedures used to synthesize Compound 7. Compound 90 was obtained as an off-white solid.

¹H NMR (400 MHz, Methanol-d₄): δ 9.20 (s, 1H), 8.88 (d, J=5.5 Hz, 1H), 7.92 (d, J=5.5 Hz, 1H), 7.78 (dd, J=7.7, 1.8 Hz, 1H), 7.58-7.49 (m, 1H), 7.41-7.16 (m, 5H), 7.16-7.12 (m, 1H), 7.10 (td, J=7.2, 1, 1H), 7.03-6.96 (m, 1H), 6.89 (td, J=7.4, 1.0 Hz, 1H), 6.49 (dd, J=7.5, 1.7 Hz, 1H), 5.59 (dd, J=10.3, 2.9 Hz, 1H), 5.43-5.22 (m, 2H), 4.42 (t, J=4.9 Hz, 2H), 4.11-3.96 (m, 2H), 3.90 (s, 3H), 3.45 (dd, J=14.1, 3.0 Hz, 1H), 3.35 (s, 1H), 3.27-3.06 (m, 8H), 2.85 (s, 3H), 2.78-2.62 (m, 1H), 2.46 (dd, J=14.0, 10.3 Hz, 1H), 2.06-1.91 (m, 3H), 1.91-1.82 (m, 2H), 1.80 (s, 3H), 1.77-1.64 (m, 2H) ppm (50 of 51 protons observed).

LC/MS: m/z=915.4 [M+H]⁺ amu.

### Example 4: Synthesis of Compounds 4-1, 4-2, 4-3, Compounds 41 Through 51, and Compounds 81, 102, 105 Through 108 and 120

### Synthesis of Compound 4-1

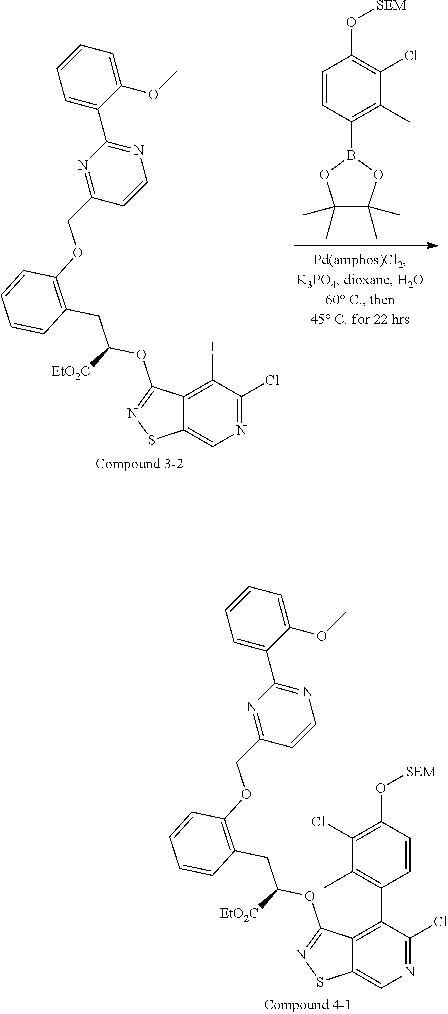

A vial was charged with Compound 3-2 (660.69 mg, 1.6 mmol), Pd(amphos)C₁₂ (37.77 mg, 0.0500 mmol) and potassium phosphate (679.53 mg, 3.2 mmol). Degassed 1,4-dioxane and water (5.2 mL, 2:1, 0.2M) was injected and the reaction was warmed to 60° C. under an atmosphere of nitrogen. After 6.5 hours, an equal portion of Compound D5 was added and the reaction was heated at 45° C. overnight. The reaction was then allowed to cool and was diluted with water and washed three times with DCM. The combined organic phase was dried over sodium sulfate, filtered and concentrated. Flash chromatography (0-100% hexanes/EtOAc) was performed and the active fractions were pooled and concentrated to dryness to yield Compound 4-1 (490 mg, 54% yield) as a yellow oil. Compound 4-1 was isolated as an approximate 4:1 mixture of atrope isomers and the major component is reported.

¹H NMR (400 MHz, Chloroform-d): δ 8.92-8.88 (m, 2H), 7.72-7.67 (m, 2H), 7.44 (ddd, J=8.9, 7.1, 1.6 Hz, 1H), 7.29 (s, 1H), 7.19 (dd, J=15.1, 8.2 Hz, 1H), 7.12-7.03 (m, 2H), 6.94 (t, J=7.4 Hz, 1H), 6.83 (d, J=8.0 Hz, 1H), 6.38-6.33 (m, 1H), 5.53 (dd, J=10.6, 2.7 Hz, 1H), 5.46 (d, J=7.0 Hz, 1H), 5.38 (d, J=7.2 Hz, 1H), 5.29-5.16 (m, 2H), 4.22 (q, J=7.1 Hz, 2H), 3.88 (s, 3H), 3.86-3.79 (m, 1H), 3.40 (dd, J=13.8, 2.8 Hz, 1H), 2.45 (dd, J=13.9, 10.5 Hz, 1H), 2.09 (s, 3H), 1.24 (s, 3H), 0.92 (ddd, J=16.6, 9.8, 7.2 Hz, 1H), −0.07 (s, 9H) ppm.

LC/MS: m/z=847.2 [M+H]⁺ amu.

### Synthesis of Compound 4-2

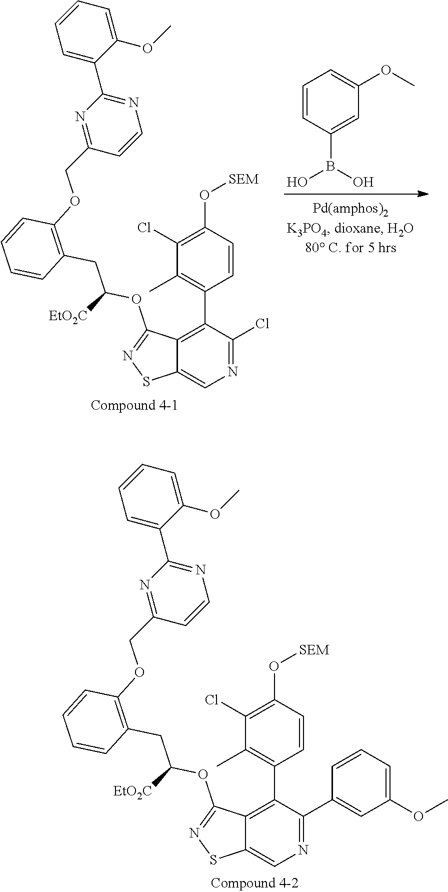 [SMILES: CCOC(=O)[C@@H](Cc1ccccc1OCc1ccnc(-c2ccccc2OC)n1)Oc1nsc2cnc(Cl)c(-c3ccc(OCOCC[Si](C)(C)C)c(Cl)c3C)c12 ; COc1cccc(B(O)O)c1 ; CCOC(=O)[C@@H](Cc1ccccc1OCc1ccnc(-c2ccccc2OC)n1)Oc1nsc2cnc(-c3cccc(OC)c3)c(-c3ccc(OCOCC[Si](C)(C)C)c(Cl)c3C)c12]

A 40 mL reaction vial was charged with potassium phosphate (369.13 mg, 1.74 mmol), Pd-X-Phos-G3 (31 mg, 0.0400 mmol), Compound 4-1 (492.1 mg, 0.5800 mmol) and 3-methoxy-phenyl-boronic acid (165 mg, 1.09 mmol). Degassed aqueous 1,4-Dioxane/water (4.5 mL, 2:1 dioxane) was injected under nitrogen and the reaction was heated to 80° C. for a period of 5 hours. The crude was diluted with water and ethyl acetate. The organic phase was separated, dried over magnesium sulfate, filtered and concentrated. Flash chromatography (20-60% Hexane/EtOAc) separated the product (Compound 4-2; 250 mg, 47% yield) from some remaining starting material (83 mg, 17% yield). Compound 4-2 was isolated as an approximate 4:1 mixture of atrope isomers, and the major component is reported.

¹H NMR (400 MHz, Chloroform-d): δ 9.27 (d, J=0.6 Hz, 1H), 8.97 (d, J=5.2 Hz, 1H), 7.80-7.73 (m, 2H), 7.56-7.45 (m, 2H), 7.32 (d, J=8.8 Hz, 1H), 7.27-7.10 (m, 5H), 7.03-6.85 (m, 5H), 6.40 (dd, J=7.5, 1.7 Hz, 1H), 5.62 (dd, J=10.3, 3.1 Hz, 1H), 5.49 (d, J=7.0 Hz, 1H), 5.41 (d, J=7.0 Hz, 1H), 5.34-5.25 (m, 2H), 4.37-4.27 (m, 2H), 3.95 (d, J=1.1 Hz, 3H), 3.86 (dt, J=10.2, 6.3 Hz, 2H), 3.72 (s, 3H), 3.44 (dd, J=13.7, 3.2 Hz, 1H), 2.60 (dd, J=13.7, 10.4 Hz, 1H), 1.86 (s, 2H), 1.64 (s, 3H), 1.34 (t, J=7.1 Hz, 3H), 0.96 (tdt, J=13.8, 10.5, 6.8 Hz, 2H), 0.00 (s, 9H) ppm.

LC/MS: m/z=919.3 [M+H]⁺ amu.

### Synthesis of Compound 4-3

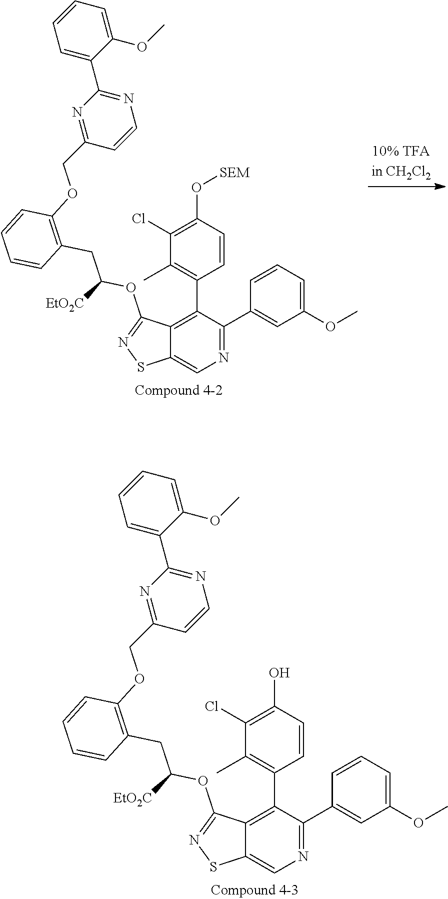 [SMILES: CCOC(=O)[C@@H](Cc1ccccc1OCc1ccnc(-c2ccccc2OC)n1)Oc1nsc2cnc(-c3cccc(OC)c3)c(-c3ccc(OCOCC[Si](C)(C)C)c(Cl)c3C)c12 ; CCOC(=O)[C@@H](Cc1ccccc1OCc1ccnc(-c2ccccc2OC)n1)Oc1nsc2cnc(-c3cccc(OC)c3)c(-c3ccc(O)c(Cl)c3C)c12]

A solution of Compound 4-2 (250 mg, 0.2700 mmol) in DCM (20 mL) was treated with 2,2,2-trifluoroacetic acid (2 mL, 24.56 mmol) (10% by volume). The reaction was monitored by TLC (1:1 Hexane/EtOAc) and once complete (30 minutes) was concentrated to dryness. The resulting TFA salt was dissolved in acetonitrile/water, frozen and lyophilized under reduced pressure to yield Compound 4-3 as a yellow powder (250 mg, quantitative). Compound 4-3 was sufficiently pure for alkylation without further purification.

¹H NMR (400 MHz, Chloroform-d): δ 9.51 (s, 1H), 9.27 (s, 1H), 8.27 (dd, J=7.9, 1.7 Hz, 1H), 8.07 (s, 1H), 7.71-7.61 (m, 1H), 7.15 (td, J=9.7, 8.8, 3.1 Hz, 4H), 7.02-6.95 (m, 2H), 6.92-6.71 (m, 5H), 6.60-6.53 (m, 1H), 5.61 (dd, J=10.3, 3.6 Hz, 1H), 5.34 (s, 2H), 4.25-4.14 (m, 2H), 4.03 (s, 3H), 3.63 (s, 3H), 3.31 (dd, J=14.6, 3.4 Hz, 1H), 2.63 (dd, J=14.6, 10.1 Hz, 1H), 1.70 (s, 3H), 1.18 (t, J=7.1 Hz, 3H) ppm.

LC/MS: m/z=919.3 [M+H]⁺ amu.

### Synthesis of Compound 41

Compound 41 was synthesized by subjecting Compound 4-3 to saponification conditions and separating atrope isomers with reverse phase chromatography following the general procedures used for Compound 7. Compound 41 was obtained as an off-white solid.

¹H NMR (400 MHz, Methanol-d₄): δ 9.11 (s, 1H), 8.75 (d, J=5.3 Hz, 1H), 7.81 (d, J=5.2 Hz, 1H), 7.51 (dd, J=7.6, 1.8 Hz, 1H), 7.38 (ddd, J=8.3, 7.4, 1.8 Hz, 1H), 7.28 (d, J=8.4 Hz, 1H), 7.11-6.94 (m, 4H), 6.88-6.76 (m, 3H), 6.75-6.63 (m, 3H), 6.32 (dd, J=7.5, 1.7 Hz, 1H), 5.42 (dd, J=10.7, 2.6 Hz, 1H), 5.15 (d, J=2.9 Hz, 2H), 3.74 (s, 3H), 3.52 (s, 3H), 3.37 (dd, J=14.1, 2.6 Hz, 1H), 2.45 (dd, J=14.3, 10.6 Hz, 1H), 1.52 (s, 3H) ppm.

LC/MS: m/z=761.2 [M+H]⁺ amu.

### Synthesis of Compound 42

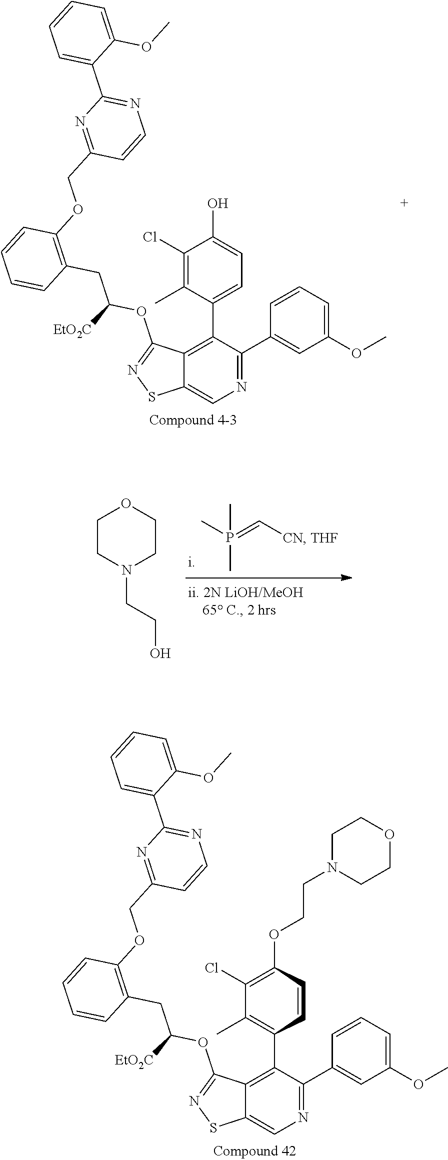

A solution of 2-morpholinoethanol (11 uL, 0.0900 mmol) in THE was prepared (150 uL in 5 mL) and transferred into a vial containing Compound 4-3 (16 mg, 0.0200 mmol). (Trimethylphoranylidine)acetonitrile solution (354.25 uL, 0.1800 mmol) was injected and the reaction was stirred at 65° C. for two hours. Once cooled, the reaction was treated with 300 uL of 2N LiOH. The reaction was stirred overnight and complete saponification to the acid occurred. The reaction was diluted with DMSO (1 mL), neutralized with acetic acid (0.08 mL, 1.33 mmol), filtered and purified by reversed phase HPLC (10-50% water/acetonitrile+0.25% AcOH buffer). Two peaks were collected with the first being the minor diastereomer (2R)-2-[4-[3-chloro-2-methyl-4-(2-morpholinoethoxy)phenyl]-5-(3-methoxyphenyl)isothiazolo[5,4-c]pyridin-3-yl]oxy-3-[2-[[2-(2-methoxyphenyl)pyrimidin-4-yl]methoxy]phenyl]propanoic acid (2.03 mg, 0.0023 mmol, 13% yield), and the second being the major diastereomer (2R)-2-[4-[3-chloro-2-methyl-4-(2-morpholinoethoxy)phenyl]-5-(3-methoxyphenyl)isothiazolo[5,4-c]pyridin-3-yl]oxy-3-[2-[[2-(2-methoxyphenyl)pyrimidin-4-yl]methoxy]phenyl]propanoic acid (Compound 42; 5.81 mg, 0.0066 mmol, 38% yield).

¹H NMR (400 MHz, Methanol-d₄): δ 9.12 (s, 1H), 8.76 (d, J=5.1 Hz, 1H), 7.82 (d, J=5.1 Hz, 1H), 7.55-7.34 (m, 3H), 7.08-6.93 (m, 5H), 6.84-6.61 (m, 5H), 6.27 (d, J=7.4 Hz, 1H), 5.35 (d, J=10.2 Hz, 1H), 5.21-5.09 (m, 2H), 4.16 (t, J=5.2 Hz, 2H), 3.76-3.72 (m, 4H), 3.57-3.48 (m, 7H), 2.73 (t, J=5.2 Hz, 2H), 2.55-2.50 (m, 5H), 1.54 (s, 3H) ppm.

LC/MS: m/z=874.3 [M+H]⁺ amu.

### Synthesis of Compound 43

Compound 43 was synthesized with the general procedures used for Compound 42 and using the corresponding alcohol. Compound 43 was obtained as an off-white amorphous solid.

¹H NMR (400 MHz, Methanol-d₄): δ 9.12 (s, 1H), 8.76 (d, J=5.3 Hz, 1H), 7.85 (d, J=5.3 Hz, 1H), 7.54-7.46 (m, 2H), 7.38 (ddd, J=8.3, 7.4, 1.8 Hz, 1H), 7.08-6.95 (m, 5H), 6.83-6.76 (m, 2H), 6.73-6.60 (m, 3H), 6.26 (dd, J=7.4, 1.8 Hz, 1H), 5.34 (dd, J=10.5, 2.6 Hz, 1H), 5.15 (d, J=4.1 Hz, 2H), 4.55-4.45 (s, 2H), 4.17 (d, J=6.3 Hz, 2H), 3.74 (s, 3H), 3.51 (s, 3H), 3.38 (dt, J=4.2, 2.1 Hz, 1H), 2.77 (t, J=5.3 Hz, 2H), 2.60-2.50 (m, 4H), 2.45 (dd, 1H, J=14.4, 10.4 Hz), 1.79 (s, 3H), 1.50-1.40 (m, 4H) ppm.

LC/MS: m/z=872.3 [M+H]⁺ amu.

### Synthesis of Compound 44

Compound 44 was synthesized with the general procedures used for Compound 42 and using the corresponding alcohol. Compound 44 was obtained as an off-white amorphous solid.

¹H NMR (400 MHz, Methanol-d₄): δ 9.12 (s, 1H), 8.75 (d, J=5.3 Hz, 1H), 7.79 (d, J=5.3 Hz, 1H), 7.50 (dt, J=7.5, 1.6 Hz, 1H), 7.38 (ddd, J=8.4, 7.4, 1.8 Hz, 1H), 7.07-6.95 (m, 4H), 6.82-6.75 (m, 2H), 6.68-6.56 (m, 3H), 6.46 (d, J=8.4 Hz, 1H), 6.37 (t, J=8.5 Hz, 1H), 5.29 (dd, J=10.6, 2.5 Hz, 1H), 5.16 (q, J=15.3 Hz, 2H), 3.96-3.86 (m, 2H), 3.74 (s, 3H), 3.64-3.57 (m, 2H), 3.51 (s, 3H), 3.32-3.27 (m, 5H), 2.62 (dd, J=15.4, 10.6 Hz, 1H), 1.81 (s, 3H) ppm.

LC/MS: m/z=819.2 [M+H]⁺ amu.

### Synthesis of Compound 45

Compound 45 was synthesized with the general procedures used for Compound 42 and using the corresponding alcohol. Compound 45 was obtained as an off-white amorphous solid.

¹H NMR (400 MHz, Methanol-d₄): δ 9.12 (s, 1H), 8.77 (d, J=5.3 Hz, 1H), 7.86 (d, J=5.3 Hz, 1H), 7.54-7.45 (m, 2H), 7.41-7.35 (m, 1H), 7.09-6.95 (m, 5H), 6.84-6.77 (m, 2H), 6.73-6.62 (m, 3H), 6.24 (dd, J=7.6, 1.7 Hz, 1H), 5.33 (dd, J=10.5, 2.6 Hz, 1H), 5.20-5.09 (m, 2H), 4.49 (s, 2H), 4.07 (d, J=6.1 Hz, 2H), 3.74 (s, 3H), 3.51 (s, 3H), 3.40-3.31 (m, 2H), 3.08-2.81 (m, 4H), 2.47-2.27 (m, 5H), 1.80 (s, 3H), 1.55 (s, 3H) ppm.

LC/MS: m/z=886.3 [M+H]⁺ amu.

### Synthesis of Compound 46

A microwave vial containing Compound 4-3 (20 mg, 0.025 mmol) was dissolved in tetrahydrofuran (0.253 mL, 0.1M). To this stirring solution was added 2-(dimethylamino)ethanol (0.010 mL, 0.101 mmol) and cyanomethylene trimethylphosphorane (0.5M in THF, 0.506 mL, 0.253 mmol). The reaction was stirred at 65° C. for 12 hours, and then allowed to cool to room temperature. To the crude reaction mixture was added lithium hydroxide in water (1N, 0.200 mL) and left to stir at room temperature for 12 hours. The reaction was quenched with acetic acid (0.200 mL) and diluted with DMSO and purified via reverse phase chromatography (0.25% AcOH/water in 20-70% acetonitrile). The product fractions were pooled and concentrated to yield Compound 46 as an off-white amorphous solid (5.4 mg, 26% yield).

¹H NMR (400 MHz, Methanol-d₄): δ 9.22 (s, 1H), 8.85 (d, J=5.3 Hz, 1H), 7.93 (d, J=5.2 Hz, 1H), 7.69-7.54 (m, 2H), 7.48 (ddd, J=8.4, 7.4, 1.8 Hz, 1H), 7.26-6.95 (m, 5H), 6.95-6.84 (m, 2H), 6.84-6.65 (m, 3H), 6.35 (dd, J=7.5, 1.7 Hz, 1H), 5.45 (dd, J=10.6, 2.5 Hz, 1H), 5.34-5.14 (m, 2H), 4.68-4.49 (m, 1H), 4.29 (t, J=5.2 Hz, 2H), 3.84 (s, 3H), 3.62 (s, 3H), 3.55-3.40 (m, 1H), 3.10-2.93 (m, J=5.1 Hz, 2H), 2.66 (s, 1H), 2.52 (s, 6H), 1.91 (s, 3H) ppm (42 of 42 protons observed).

LC/MS: m/z=832.3 [M+H]⁺ amu.

### Synthesis of Compound 47

Compound 47 was synthesized with the general procedures used for Compound 42 and using the corresponding alcohol. Compound 47 was obtained as an off-white amorphous solid.

¹H NMR (400 MHz, Methanol-d₄): δ 9.12 (s, 1H), 8.77 (d, J=5.2 Hz, 1H), 7.85 (d, J=5.2 Hz, 1H), 7.51 (dd, J=7.6, 1.8 Hz, 1H), 7.44-7.31 (m, 2H), 7.13-6.93 (m, 5H), 6.84-6.77 (m, 2H), 6.75-6.60 (m, 3H), 6.34 (dd, J=7.6, 1.7 Hz, 1H), 5.38 (dd, J=10.0, 3.1 Hz, 1H), 5.23-5.06 (m, 2H), 4.10 (dq, J=11.4, 5.9, 5.2 Hz, 2H), 3.74 (s, 3H), 3.52 (s, 3H), 3.40-3.30 (m, 1H), 2.86 (t, J=7.6 Hz, 2H), 2.55-2.40 (m, 7H), 2.09-1.94 (m, 2H), 1.81 (s, 3H) ppm.

LC/MS: m/z=846.3 [M+H]⁺ amu.

### Synthesis of Compound 48

Compound 48 was synthesized with the general procedures used for Compound 42 and using the corresponding alcohol. Compound 48 was obtained as an off-white amorphous solid.

¹H NMR (400 MHz, Methanol-d₄): δ 9.12 (s, 1H), 8.76 (dd, J=5.3, 3.1 Hz, 1H), 7.85 (t, J=4.8 Hz, 1H), 7.55-7.45 (m, 2H), 7.38 (ddd, J=8.9, 7.4, 1.8 Hz, 1H), 7.10-6.93 (m, 5H), 6.85-6.75 (m, 2H), 6.73-6.62 (m, 3H), 6.25 (dd, J=9.6, 7.4 Hz, 1H), 5.40-5.30 (m, 1H), 5.18-5.10 (m, 2H), 4.49 (s, 1H), 4.23-4.07 (m, 1H), 3.74 (s, 3H), 3.52 (d, J=3.5 Hz, 3H), 3.42-3.34 (m, 1H), 2.73-2.27 (m, 5H), 1.80 (s, 5H), 1.62-1.42 (m, 4H) ppm.

LC/MS: m/z=858.3 [M+H]⁺ amu.

### Synthesis of Compound 49

Compound 49 was synthesized with the general procedures used for Compound 42 and using the corresponding alcohol. Compound 49 was obtained as an off-white amorphous solid.

¹H NMR (400 MHz, Methanol-d₄): δ 9.12 (s, 1H), 8.77 (dd, J=5.3, 1.2 Hz, 1H), 7.84 (dd, J=15.1, 5.3 Hz, 1H), 7.57-7.49 (m, 2H), 7.47-7.35 (m, 2H), 7.09-6.93 (m, 5H), 6.84-6.76 (m, 2H), 6.73-6.62 (m, 3H), 6.30-6.25 (m, 1H), 5.44-5.29 (m, 1H), 5.15 (s, 2H), 4.51 (s, 1H), 4.13-3.85 (m, 1H), 3.74 (s, 3H), 3.51 (s, 3H), 3.42-3.34 (m, 1H), 2.73-2.27 (m, 5H), 1.80 (s, 5H), 1.62-1.42 (m, 4H) ppm.

LC/MS: m/z=858.3 [M+H]⁺ amu.

### Synthesis of Compound 50

Compound 50 was synthesized with the general procedures used for Compound 42 and using the corresponding alcohol. Compound 50 was obtained as an off-white amorphous solid.

¹H NMR (400 MHz, Methanol-d₄): δ 9.13 (s, 1H), 8.69 (d, J=5.2 Hz, 1H), 7.78 (d, J=5.2 Hz, 1H), 7.60-7.45 (m, 2H), 7.38 (ddd, J=8.4, 7.4, 1.8 Hz, 1H), 7.13-6.87 (m, 5H), 6.83-6.66 (m, 4H), 6.64-6.57 (m, 1H), 6.19 (dd, J=7.5, 1.7 Hz, 1H), 5.28 (dd, J=10.3, 2.7 Hz, 1H), 5.18-4.95 (m, 2H), 4.33 (t, J=5.1 Hz, 2H), 3.74 (s, 3H), 3.52 (s, 3H), 3.46-3.38 (m, 2H), 3.35-3.28 (m, 1H), 3.20 (s, 4H), 2.45-2.29 (m, 1H), 1.88 (t, J=4.5 Hz, 4H), 1.60 (s, 3H) ppm.

LC/MS: m/z=858.3 [M+H]⁺ amu.

### Synthesis of Compound 51

Compound 51 was synthesized with the general procedures used for Compound 42 and using the corresponding alcohol. Compound 51 was obtained as an off-white amorphous solid.

¹H NMR (400 MHz, Methanol-d₄): δ 9.15 (s, 1H), 8.75 (d, J=5.3 Hz, 1H), 7.75 (d, J=5.2 Hz, 1H), 7.51 (dd, J=7.6, 1.8 Hz, 1H), 7.44-7.32 (m, 2H), 7.16-6.94 (m, 5H), 6.88-6.79 (m, 2H), 6.78-6.65 (m, 3H), 6.31 (dd, J=7.6, 1.7 Hz, 1H), 5.46 (dd, J=10.4, 2.8 Hz, 1H), 5.16 (d, J=4.7 Hz, 2H), 3.80 (s, 3H), 3.74 (s, 3H), 3.52 (s, 3H), 3.43-3.27 (m, 2H), 2.43 (dd, J=14.3, 10.4 Hz, 1H), 1.58 (s, 3H) ppm.

LC/MS: m/z=775.2 [M+H]⁺ amu.

### Synthesis of Compound 81

Compound 81 was synthesized with the general procedures used for Compound 42 using the corresponding aryl boronate ester or acid when performing the procedures used for the synthesis of Compound 4-2 and using the corresponding alcohol in the final step. Compound 81 was obtained as an off-white amorphous solid.

¹H NMR (400 MHz, Methanol-d₄): δ 9.12 (s, 1H), 8.77 (d, J=5.2 Hz, 1H), 7.86 (d, J=5.2 Hz, 1H), 7.53-7.47 (m, 2H), 7.42-7.35 (m, 1H), 7.20 (dd, J=8.8, 5.4 Hz, 2H), 7.06 (d, J=8.4 Hz, 1H), 7.02-6.95 (m, 4H), 6.86 (t, J=8.8 Hz, 2H), 6.78 (d, J=8.2 Hz, 1H), 6.65 (t, J=7.4 Hz, 1H), 6.21 (d, J=6.9 Hz, 1H), 5.32 (dd, J=10.6, 2.6 Hz, 1H), 5.23-5.09 (m, 2H), 4.49 (s, 5H), 4.09 (d, J=5.0 Hz, 2H), 3.74 (s, 4H), 3.44-3.33 (m, 3H), 3.03 (p, J=1.6 Hz, 2H), 2.39 (dd, J=14.3, 10.5 Hz, 1H), 2.30 (s, 2H), 1.80 (s, 6H), 1.64 (s, 2H), 1.53 (s, 3H) ppm.

LC/MS: m/z=874.3 [M+H]⁺ amu.

### Synthesis of Compound 102

Compound 102 was synthesized with the general procedures used for Compound 42 using the corresponding aryl boronate ester or acid when performing the procedures used for the synthesis of Compound 4-2 and using the corresponding alcohol in the final step. Compound 102 was obtained as an off-white amorphous solid.

¹H NMR (400 MHz, Methanol-d₄): δ 9.20 (s, 1H), 8.75 (d, J=5.3 Hz, 1H), 8.02-7.90 (m, 1H), 7.72 (d, J=5.3 Hz, 1H), 7.53 (dd, J=7.6, 1.7 Hz, 1H), 7.47-7.20 (m, 3H), 7.20-6.83 (m, 11H), 6.73 (t, J=7.4 Hz, 1H), 6.30 (d, J=7.6 Hz, 1H), 5.45 (dd, J=10.1, 3.2 Hz, 1H), 5.26-5.06 (m, 2H), 4.12 (dt, J=10.8, 5.9 Hz, 2H), 3.75 (s, 3H), 3.38 (p, J=1.7 Hz, 1H), 3.03 (p, J=1.6 Hz, 1H), 2.91 (d, J=11.4 Hz, 1H), 2.85-2.63 (m, 5H), 2.41 (dd, J=14.0, 10.1 Hz, 1H) ppm.

LC/MS: m/z=874.3 [M+H]⁺ amu.

### Synthesis of Compound 107

Compound 107 was synthesized by first deprotecting Compound 4-1 following the general procedures for Compound 4-3, then using the resulting product with the corresponding alcohol while following the first step (i.e., step “i.”) for the synthesis of Compound 42, and lastly, using that product and cyclobutyl zinc bromide while following the general procedures for the synthesis of Compound 29. Compound 107 was obtained as an off-white solid.

LC/MS: m/z=723.2 [M+H]⁺ amu.

### Synthesis of Compound 108

Compound 108 was synthesized by first deprotecting Compound 4-1 following the general procedures for Compound 4-3, then using the resulting product with the corresponding alcohol while following the first step (i.e., step “i.”) for the synthesis of Compound 42, and lastly, using that product and cyclobutyl zinc bromide while following the general procedures for the synthesis of Compound 29. Compound 108 was obtained as an off-white solid.

LC/MS: m/z=822.3 [M+H]⁺ amu.

### Synthesis of Compound 105

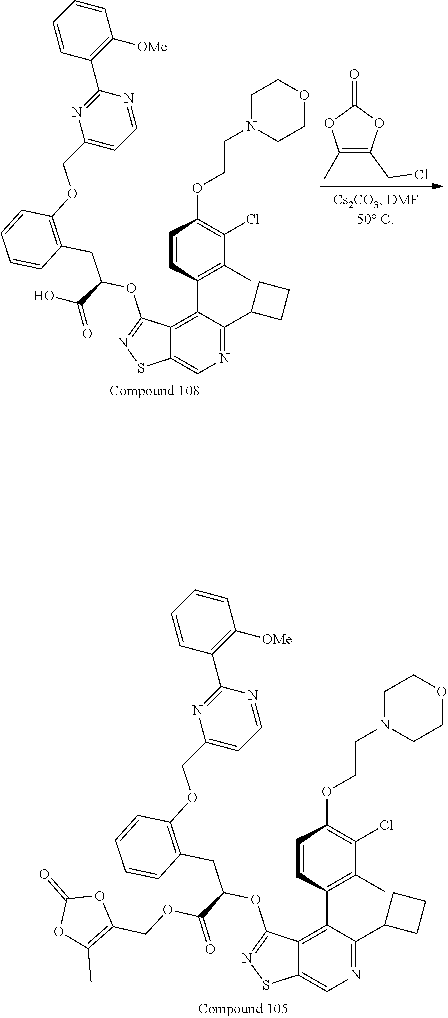

To a vial containing Compound 108 (46.0 mg, 0.060 mmol) was added cesium carbonate (36.5 mg, 0.110 mmol). The solids were dissolved in dimethylformamide (2.24 mL). To the stirring solution was added 4-(chloromethyl)-5-methyl-1,3-dioxol-2-one (14.1 mg, 0.100 mmol). The reaction was capped and heated to 50° C. for 1 hour, allowed to cool to room temperature and poured into a separatory funnel containing water (1 mL). The organic phase was separated and the aqueous phase was washed with dichloromethane (2 mL, 2 times), dried over sodium sulfate, filtered, and concentrated in vacuo. The crude residue was purified via silica gel chromatography (0 to 20% methanol in dichloromethane). The product fractions were pooled and concentrated to yield Compound 105 as an off-white oil.

¹H NMR (400 MHz, Methanol-d₄): δ 9.23 (s, 1H), 8.86 (d, J=5.2 Hz, 1H), 7.68 (d, J=5.2 Hz, 1H), 7.62 (dd, J=7.6, 1.8 Hz, 1H), 7.47 (ddd, J=8.3, 7.4, 1.8 Hz, 1H), 7.20 (ddd, J=8.2, 7.4, 1.7 Hz, 1H), 7.15 (dd, J=8.5, 1.0 Hz, 1H), 7.12-7.08 (m, 2H), 7.06 (td, J=7.5, 0.9 Hz, 1H), 6.98 (dd, J=8.4, 1.1 Hz, 1H), 6.88 (td, J=7.5, 1.1 Hz, 1H), 6.61 (dd, J=7.5, 1.7 Hz, 1H), 5.56 (dd, J=9.0, 4.8 Hz, 1H), 5.34-5.14 (m, 2H), 4.35-4.21 (m, 2H), 3.83 (s, 3H), 3.69-3.60 (m, 4H), 3.60-3.50 (m, 1H), 3.12 (dd, J=13.9, 4.8 Hz, 1H), 2.96-2.79 (m, 2H), 2.75-2.60 (m, 5H), 2.56-2.35 (m, 2H), 2.15 (s, 2H), 2.08-1.97 (s, 5H), 1.95-1.73 (m, 5H) (48 of 48 protons observed).

LC/MS: m/z=934.3 [M+H]⁺ amu.

### Synthesis of Compound 106

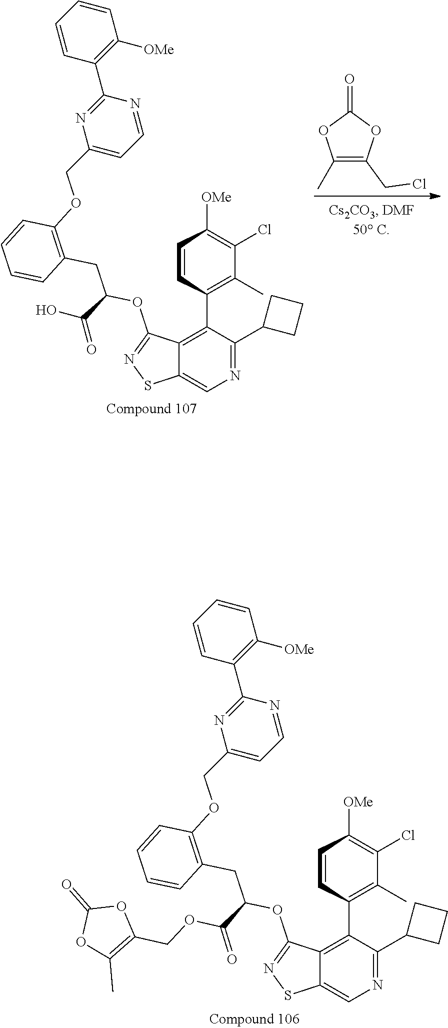

To a vial containing Compound 107 (75.0 mg, 0.10 mmol) was added cesium carbonate (67.6 mg, 0.210 mmol). The solids were dissolved in dimethylformamide (4.18 mL). To the stirring solution was added 4-(chloromethyl)-5-methyl-1,3-dioxol-2-one (26.2 mg, 0.18 mmol). The reaction was capped and heated to 50° C. for 1 hour, allowed to cool and poured into a separatory funnel containing water (1 mL). The organic phase was separated and the aqueous phase was washed with dichloromethane (2 mL, 2 times). The organic phase was separated and the aqueous phase was washed with dichloromethane (2 mL, 2 times), dried over sodium sulfate, filtered, and concentrated in vacuo. The crude residue was purified via silica gel chromatography (0 to 20% methanol in dichloromethane). The product fractions were pooled and concentrated to yield Compound 106 as an off-white oil.

¹H NMR (500 MHz, Methanol-d₄): δ 9.23 (s, 1H), 8.86 (d, J=5.2 Hz, 1H), 7.97 (s, 4H), 7.69 (d, J=5.2 Hz, 1H), 7.61 (dd, J=7.5, 1.8 Hz, 1H), 7.56-7.45 (m, 1H), 7.23-7.13 (m, 2H), 7.11-7.03 (m, 3H), 6.97 (d, J=8.2 Hz, 1H), 6.88 (t, J=7.5 Hz, 1H), 6.60 (dd, J=7.3, 1.7 Hz, 1H), 5.59 (dd, J=9.2, 4.5 Hz, 1H), 5.32-5.18 (m, 2H), 4.95 (d, J=14.0 Hz, 1H), 4.85 (d, J=14.0 Hz, 1H), 3.95 (s, 3H), 3.84 (s, 3H), 3.54 (p, J=8.7 Hz, 1H), 3.14 (dd, J=13.8, 4.7 Hz, 1H), 2.74-2.64 (m, 1H), 2.55-2.34 (m, 2H), 2.16 (s, 1H), 2.06 (s, 3H), 2.04-1.96 (m, 2H) (39 of 39 protons observed).

LC/MS: m/z=835.2 [M+H]⁺ amu.

### Synthesis of Compound 120

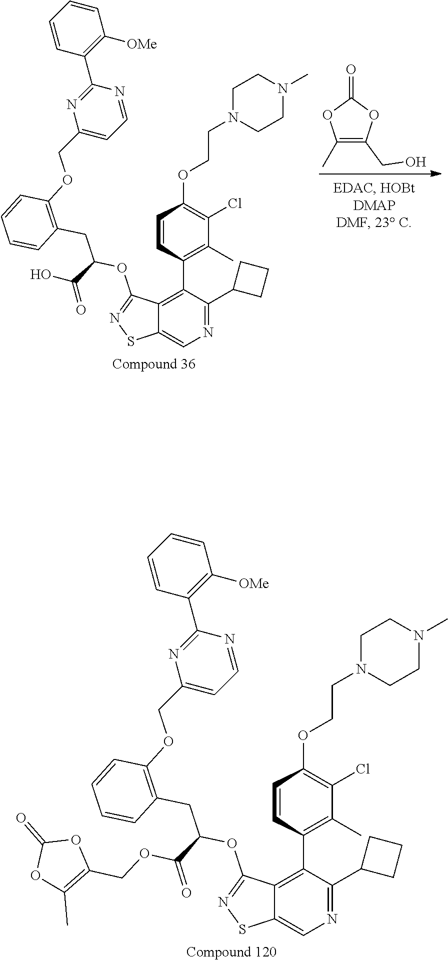

To a vial containing Compound 36 (60.0 mg, 0.072 mmol) was added N-(3-dimethylaminopropyl)-N′-ethylcarbodiimide hydrochloride (19.3 mg, 0.110 mmol), 1-hydroxybenzotriazole hydrate (13.6 mg, 0.100 mmol), 4-(dimethylamino)pyridine (12.3 mg, 0.100 mmol). The solids were dissolved in dimethylformamide (0.720 mL). To the stirring solution was added 4-(hydroxymethyl)-5-methyl-1,3-dioxol-2-one (14.0 mg, 0.108 mmol). The reaction was capped and stirred at 23° C. for 1 hour. After the reaction was complete the reaction was poured into a separatory funnel containing water (1 mL). The organic phase was separated and the aqueous phase was washed with dichloromethane (2 mL, 2 times). The combined organic extracts were concentrated to dryness. The crude reaction mixture was purified via reverse phase chromatography (0-60% acetonitrile in water with 0.025% AcOH). The product fractions were pooled and concentrated to yield Compound 120 as a white solid.

¹H NMR (400 MHz, Methanol-d₄): δ 9.24 (s, 1H), 8.87 (d, J=5.2 Hz, 1H), 7.69 (d, J=5.2 Hz, 1H), 7.62 (dd, J=7.5, 1.8 Hz, 1H), 7.55-7.39 (m, 1H), 7.25-7.18 (m, 1H), 7.18-7.14 (m, 1H), 7.13-7.03 (m, 3H), 7.03-6.96 (m, 1H), 6.89 (td, J=7.4, 1.0 Hz, 1H), 6.62 (dd, J=7.5, 1.7 Hz, 1H), 5.56 (dd, J=9.0, 4.8 Hz, 1H), 5.37-5.15 (m, 3H), 4.59 (s, 2H), 4.30 (qt, J=10.4, 5.0 Hz, 3H), 3.84 (s, 4H), 3.59-3.45 (m, 1H), 3.18-3.06 (m, 2H), 2.93 (t, J=5.2 Hz, 3H), 2.87-2.54 (m, 8H), 2.54-2.31 (m, 6H), 2.05 (s, 3H), 1.97-1.91 (m, 2H) (51 of 51 protons observed).

LC/MS: m/z=947.3 [M+H]⁺ amu.

### Example 5: Synthesis of Intermediate 5-1, Compound 5-1, and Compounds 55 and 56

### Synthesis of Intermediate 5-1

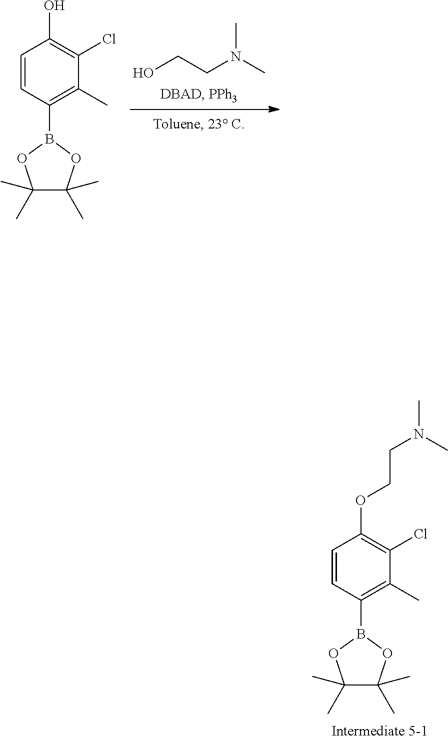

To a mixture of 2-chloro-3-methyl-4-(4,4,5,5-tetramethyl-1,3,2-dioxaborolan-2-yl)phenol (1.0 g, 3.72 mmol), triphenylphosphine (1.4 g, 5.59 mmol), and 2-(dimethylamino)ethanol (0.45 mL, 4.47 mmol) in toluene (12 mL) was added di-tert-butyl azodicarboxylate (1.3 g, 5.59 mmol) in toluene (6 mL) dropwise to the stirring reaction solution. The reaction was stirred at 23° C. for 2 hours, at which time LC/MS analysis showed complete conversion to the desired product (LC/MS: m/z=340.1 [M+H]⁺ amu).

The reaction was stopped and concentrated onto silica gel. Silica gel chromatography was performed (0-15% methanol/dichloromethane). The product fractions were pooled and concentrated to yield Intermediate 5-1 as a clear oil (0.555 g, 44% yield).

### Synthesis of Compound 5-1

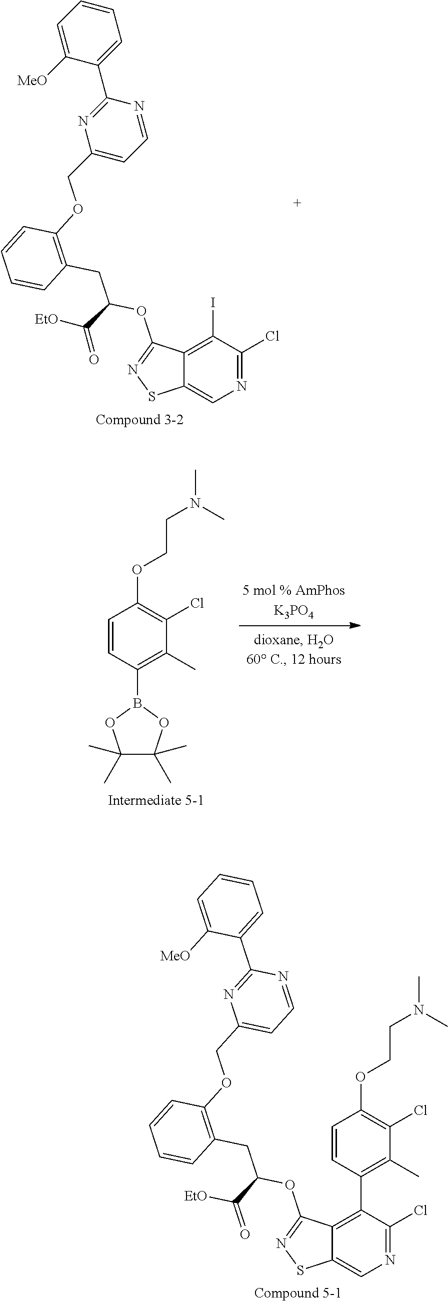

A flask containing Compound 3-2 (0.400 mg, 0.57 mmol) was charged with Intermediate 5-1 (0.203 g, 0.60 mmol), bis(di-tert-butyl(4-dimethylaminophenyl)phosphine)dichloropalladium(II) (20 mg, 0.029 mmol), and potassium phosphate tribasic (0.362 g, 1.71 mmol). The solids were dissolved in degassed 1,4-dioxane (1.75 mL) and deionized water (0.88 mL). The reaction was stirred at 60° C. for 12 hours, allowed to cool and poured into a separatory funnel containing water (3 mL). The organic phase was separated and the aqueous phase was washed with dichloromethane (5 mL, 2 times). The combined organic extracts were concentrated onto silica gel. Silica gel chromatography was performed (0-20% methanol/dichloromethane). The product fractions were pooled and concentrated to yield Compound 5-1 as a yellow solid and a mixture of diastereomers (LC/MS: m/z=788.2 [M+H]⁺ amu).

### Synthesis of Compound 55

Compound 55 was synthesized following the general procedures used for Compound 7, using Compound 5-1 and the corresponding aryl boronate ester or acid as the starting materials. Compound 55 was obtained as an off-white solid.

¹H NMR (400 MHz, Methanol-d₄): δ 9.24 (s, 1H), 8.80 (d, J=5.2 Hz, 1H), 7.88 (d, J=5.2 Hz, 1H), 7.68-7.54 (m, 2H), 7.48 (ddd, J=9.0, 7.5, 1.8 Hz, 1H), 7.26-7.11 (m, 3H), 7.11-7.02 (m, 2H), 7.02-6.93 (m, 3H), 6.80 (d, J=8.1 Hz, 1H), 6.66 (t, J=7.4 Hz, 1H), 6.24 (dd, J=7.5, 1.6 Hz, 1H), 5.38 (dd, J=10.4, 2.4 Hz, 1H), 5.22-5.04 (m, 2H), 4.47-4.36 (m, 2H), 3.84 (s, 3H), 3.39 (d, J=13.9 Hz, 1H), 2.76 (s, 6H), 2.44 (dd, J=14.1, 10.5 Hz, 1H), 1.93 (s, 3H), 1.66 (s, 3H) ppm (39 of 39 protons observed).

LC/MS: m/z=820.3 [M+H]⁺ amu.

### Synthesis of Compound 56

Compound 56 was synthesized using Compound 5-1 and cyclobpropylzinc bromide as starting materials, and following the general procedures used to synthesize Compound 29. Compound 56 was obtained as an off-white solid.

¹H NMR (500 MHz, Methanol-d₄): δ 9.03 (s, 1H), 8.82 (d, J=5.3 Hz, 1H), 7.84 (d, J=5.3 Hz, 1H), 7.64 (dd, J=7.5, 1.8 Hz, 1H), 7.57-7.47 (m, 1H), 7.34 (d, J=8.5 Hz, 1H), 7.28-7.22 (m, 1H), 7.20-7.15 (m, 2H), 7.08 (t, J=7.5 Hz, 1H), 6.99 (d, J=8.2 Hz, 1H), 6.91 (t, J=7.4 Hz, 1H), 6.48 (dd, J=7.6, 1.7 Hz, 1H), 5.56 (dd, J=10.2, 2.9 Hz, 1H), 5.28 (q, J=15.0 Hz, 2H), 4.58-4.50 (m, 2H), 3.85 (s, 3H), 3.74-5.68 (m, 3H), 3.48-3.39 (m, 1H), 3.08 (m, 4H), 2.44 (dd, J=13.9, 10.2 Hz, 1H), 2.06 (s, 3H), 1.77-1.62 (m, 1H), 1.29 (s, 2H), 1.16-0.74 (m, 4H) ppm (40 of 40 protons observed).

LC/MS: m/z=766.2 [M+H]⁺ amu.

### Example 6: Synthesis of Intermediate 6-1, Compound 6-1, Compound 6-2 Compounds 57 Through 63 and Compound 104

### Synthesis of Intermediate 6-1

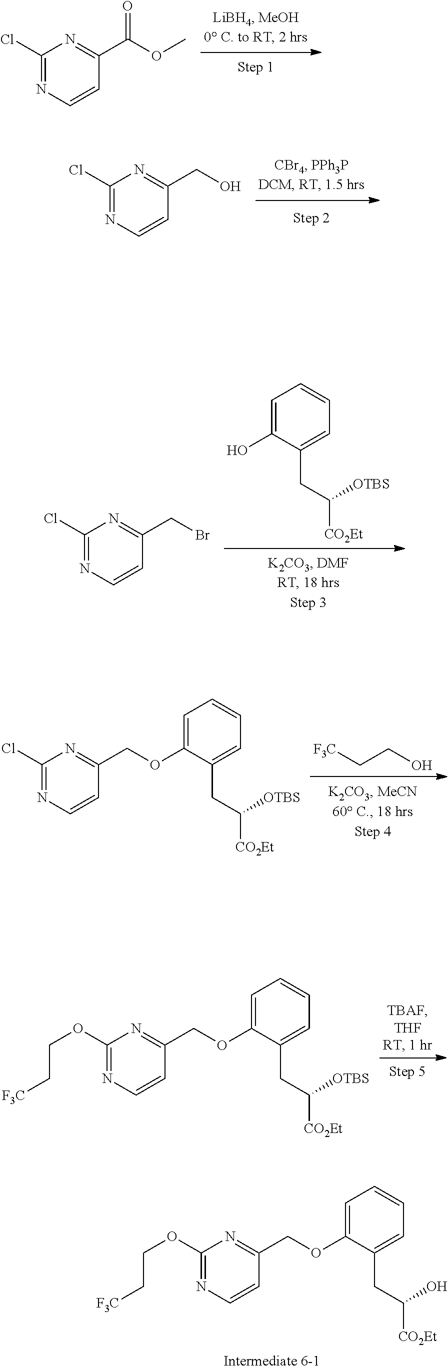

### Step 1

To an oven-dry 250 mL flask was added methyl 2-chloropyrimidine-4-carboxylate (5. g, 28.97 mmol) and Methanol (100 mL). The resulting mixture was cooled to 0° C. followed by the addition of LiBH₄ (16. mL, 32 mmol) dropwise via an addition funnel. The reaction was allowed to warm to ambient temperature and continued for one hour. The mixture was quenched with water (2 mL) and the solvent was removed under reduced pressure. The resulting residue was partitioned between EtOAc (80 mL) and water (40 mL). The aqueous layer was extracted with 20% iPrOH in CHCl₃ (2×40 mL). The combined organic layers were dried over MgSO₄, filtered, and concentrated. The crude product was purified by silica gel chromatography to give the desired product as a yellow solid.

¹H NMR (300 MHz, CDCl₃): δ 8.63 (dd, J=5.1, 0.6 Hz, 1H), 7.39 (dt, J=5.1, 0.7 Hz, 1H), 4.81 (d, J=0.8 Hz, 2H) ppm.

LC/MS: m/z=145.3 [M+H]⁺ amu.

### Step 2

To a 250 mL flask, which contained (2-chloropyrimidin-4-yl)methanol (2.6 g, 17.99 mmol), was added carbon tetrabromide (8.95 g, 26.98 mmol) and DCM (104 mL). The resulting mixture was cooled to 0° C. Triphenylphosphane (9.43 g, 35.97 mmol) in DCM (15 mL) was added to the mixture. The ice bath was removed; the reaction mixture was warmed to ambient temperature. After 45 minutes, the mixture was adsorbed onto silica and purified by silica gel chromatography to give the desired product as a light brown liquid.

¹H NMR (300 MHz, CDCl₃): δ 8.67 (d, J=5.0 Hz, 1H), 7.58-7.37 (m, 1H), 4.44 (s, 2H) ppm.

LC/MS: m/z=207.2 [M+H]⁺ amu.

### Step 3

To a mixture of ethyl (2S)-2-[tert-butyl(dimethyl)silyl]oxy-3-(2-hydroxyphenyl)propanoate (3.91 g, 12.05 mmol) and K₂CO₃ (3.33 g, 24.1 mmol) in DMF (60 mL) was added 4-(bromomethyl)-2-chloro-pyrimidine (2.5 g, 12.05 mmol) in DMF (20 mL). The resulting mixture was stirred at ambient temperature under inert atmosphere for 12 hours at which point the mixture was partitioned between EtOAc (70 mL) and water (30 mL). The organic layer was washed with water (2×20 mL), brine (10 mL), dried over MgSO₄, and concentrated. The crude product was purified by silica gel chromatography to give the desired product as a white solid.

¹H NMR (300 MHz, CDCl₃) δ 8.71 (d, J=5.1 Hz, 1H), 7.77 (dt, J=5.1, 0.9 Hz, 1H), 7.24 (ddd, J=6.8, 5.3, 1.8 Hz, 2H), 6.98 (td, J=7.5, 1.1 Hz, 1H), 6.84 (d, J=8.3 Hz, 1H), 5.18 (t, J=1.1 Hz, 2H), 4.51 (dd, J=9.6, 3.7 Hz, 1H), 4.23 (qd, J=7.1, 2.6 Hz, 2H), 3.36 (dd, J=13.2, 3.7 Hz, 1H), 2.92 (dd, J=13.2, 9.6 Hz, 1H), 1.31 (t, J=7.1 Hz, 3H), 0.77 (s, 9H), −0.14 (s, 3H), −0.26 (s, 3H) ppm.

LC/MS: m/z=451.2 [M+H]⁺ amu.

### Step 4

To a solution of ethyl (2S)-2-[tert-butyl(dimethyl)silyl]oxy-3-[2-[(2-chloropyrimidin-4-yl)methoxy]phenyl]propanoate (120 mg, 0.2700 mmol) in MeCN (2 mL) was added 2,2-difluoroethanol (0.5 mL, 7.11 mmol) and K₂CO₃ (73.54 mg, 0.5300 mmol). The resulting mixture was heated at 60° C. under inert atmosphere for 17 hours. The mixture was cooled to ambient temperature, quenched with water (5 mL), partitioned between EtOAc (40 mL) and water (20 mL) and extracted with EtOAc (20 mL). The combined organic layers were dried over MgSO₄, filtered, and concentrated under reduced pressure, and used without further purification.

¹H NMR (300 MHz, CDCl₃): δ 8.58 (d, J=5.0 Hz, 1H), 7.43 (d, J=5.0 Hz, 1H), 7.27-7.18 (m, 2H), 6.96 (dd, J=8.1, 7.1 Hz, 1H), 6.84 (d, J=8.3 Hz, 1H), 5.12 (s, 2H), 4.64 (t, J=6.7 Hz, 2H), 4.53 (dd, J=9.5, 3.9 Hz, 1H), 4.28-4.18 (m, 2H), 3.37 (dd, J=13.1, 3.9 Hz, 1H), 2.92 (dd, J=13.1, 9.5 Hz, 1H), 2.80-2.63 (m, 2H), 1.34-1.26 (m, 3H), 0.77 (d, J=0.4 Hz, 9H), −0.14 (s, 3H), −0.26 (s, 3H) ppm.

¹⁹F NMR (282 MHz, CDCl₃): δ-62.99-−66.90 (m).

LC/MS: m/z=529.1 [M+H]⁺ amu.

### Step 5

To a solution of ethyl (2S)-2-[tert-butyl(dimethyl)silyl]oxy-3-[2-[[2-(3,3,3-trifluoropropoxy)pyrimidin-4-yl]methoxy]phenyl]propanoate (110 mg, 0.2100 mmol) in THE (2 mL) was added tertbutyl ammonium fluoride solution (1 M in THF, 0.33 mL, 0.3300 mmol). The resulting mixture was stirred at ambient temperature. The reaction mixture was then purified by silica gel chromatography to give Intermediate 6-1 as a white foam.

¹H NMR (300 MHz, CDCl₃): δ 8.56 (d, J=5.0 Hz, 1H), 7.33 (dd, J=5.0, 0.7 Hz, 1H), 7.25 (t, J=7.0 Hz, 2H), 6.99 (t, J=7.4 Hz, 1H), 6.84 (d, J=8.5 Hz, 1H), 5.14 (s, 2H), 4.69-4.61 (m, 2H), 4.56 (dd, J=8.2, 4.7 Hz, 1H), 4.32-4.15 (m, 2H), 3.33 (dd, J=13.7, 4.7 Hz, 1H), 3.04 (dd, J=13.7, 8.2 Hz, 1H), 2.80-2.57 (m, 2H), 1.28 (td, J=7.2, 0.6 Hz, 3H) ppm.

### 19F NMR (282 MHz, CDCl₃): δ-64.80 (t, J=10.6 Hz).

LC/MS: m/z=415.1 [M+H]⁺ amu.

### Synthesis of Compound 57

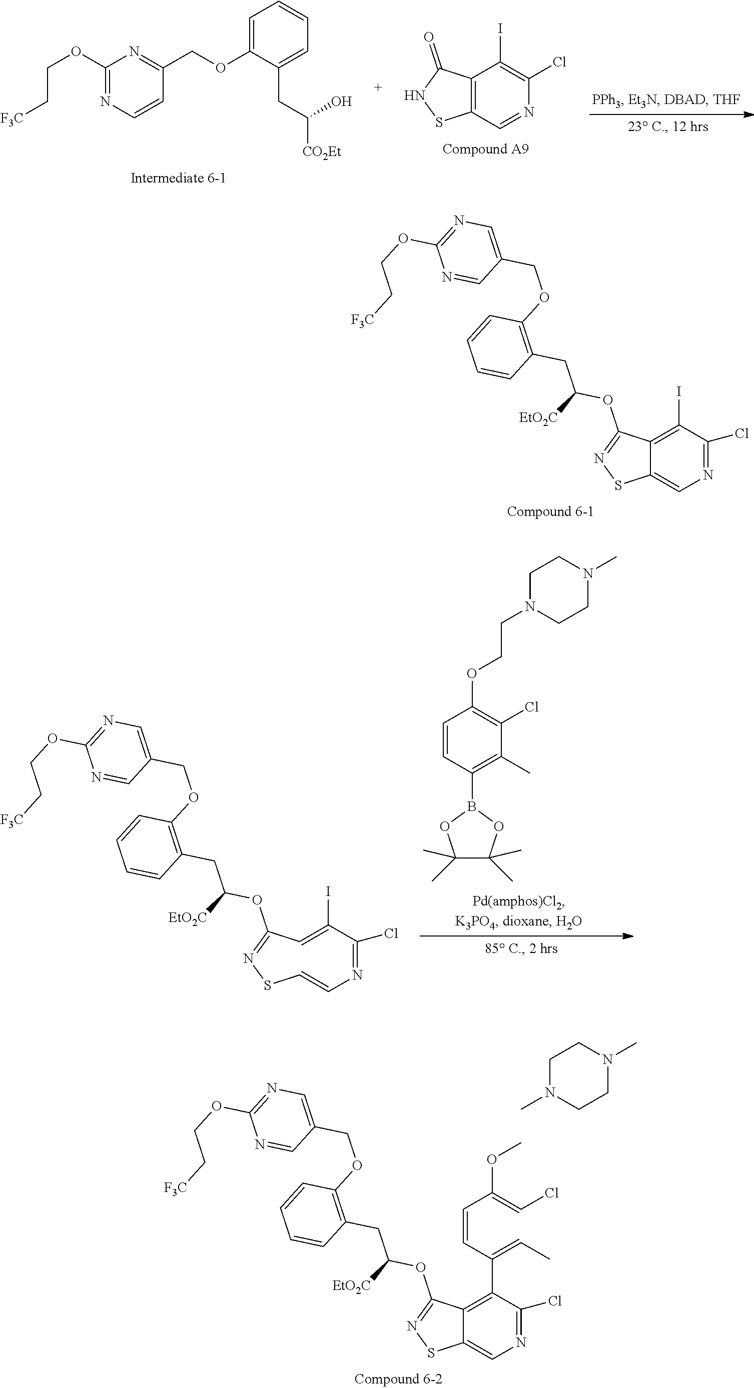

Compound 6-1 was synthesized using Compound A9, Intermediate 6-1, and following the general procedures used to synthesize Compound 3-2. Then, Compound 6-2 was synthesized using Compound B-6, Compound 6-1, and following the general procedures used to synthesize Compound 3-3. Compound 57 was synthesized using Compound 6-2 and following the general procedures used to synthesize Compound 39.

¹H NMR (300 MHz, Methanol-d₄): δ 9.33 (s, 1H), 8.62 (d, J=5.1 Hz, 1H), 7.53 (d, J=5.1 Hz, 1H), 7.44 (d, J=8.5 Hz, 1H), 7.24-7.13 (m, 3H), 6.95 (dd, J=9.8, 8.3 Hz, 2H), 6.90-6.78 (m, 3H), 6.41 (d, J=6.2 Hz, 1H), 5.59 (dd, J=10.2, 3.1 Hz, 1H), 5.19 (d, J=6.3 Hz, 2H), 4.98 (ddtd, J=4.0, 1.6, 0.8, 0.4 Hz, 2H), 4.93-4.87 (m, 1H), 4.67 (t, J=6.2 Hz, 2H), 4.35 (t, J=5.0 Hz, 2H), 3.68 (s, 3H), 3.44-3.38 (m, 3H), 3.33-3.21 (m, 3H), 3.10 (t, J=4.7 Hz, 3H), 2.86 (s, 3H), 2.77 (dt, J=10.9, 6.1 Hz, 2H), 1.78 (s, 3H) ppm.

¹⁹F NMR (282 MHz, Methanol-d₄): δ-66.31 (t, J=10.9 Hz), −77.37.

LC/MS: m/z=893.1 [M+H]⁺ amu.

### Synthesis of Compound 58

Compound 58 was synthesized following the general procedures used for Intermediate 6-1 using the corresponding fluorinated alcohol, and then following the general procedures used for Compound 57. Compound 58 was obtained as an amorphous off-white solid.

¹H NMR (300 MHz, Methanol-d₄): δ 9.31 (s, 1H), 8.62 (d, J=5.1 Hz, 1H), 7.55 (d, J=5.1 Hz, 1H), 7.41 (d, J=8.5 Hz, 1H), 7.22-7.11 (m, 3H), 6.98-6.88 (m, 2H), 6.87-6.78 (m, 3H), 6.44-6.02 (m, 4H), 5.56 (dd, J=10.2, 3.0 Hz, 1H), 5.18 (d, J=6.2 Hz, 2H), 4.64 (td, J=14.0, 3.9 Hz, 2H), 4.32 (t, J=4.6 Hz, 2H), 3.66 (s, 3H), 3.46-3.33 (m, 4H), 3.29-3.18 (m, 3H), 3.11-3.01 (m, 3H), 2.84 (s, 3H), 2.48 (dd, J=13.9, 10.2 Hz, 2H), 1.76 (s, 3H) ppm.

¹⁹F NMR (282 MHz, Methanol-d₄): δ-77.34.

LC/MS: m/z=860.3 [M+H]⁺ amu.

### Synthesis of Compound 59

Compound 59 was synthesized following the general procedures used for Intermediate 6-1 using the corresponding fluorinated alcohol, and then following the general procedures used for Compound 57. Compound 59 was obtained as an amorphous off-white solid.

¹H NMR (300 MHz, Methanol-d₄): δ 9.30 (s, 1H), 8.57 (d, J=5.1 Hz, 1H), 7.47 (d, J=5.1 Hz, 1H), 7.41 (d, J=8.5 Hz, 1H), 7.22-7.10 (m, 3H), 6.92 (t, J=8.3 Hz, 2H), 6.84 (d, J=9.6 Hz, 2H), 6.39 (d, J=6.8 Hz, 1H), 5.56 (dd, J=10.1, 3.0 Hz, 1H), 5.29-5.21 (m, 1H), 5.15 (d, J=6.0 Hz, 1H), 4.89-4.84 (m, 1H), 4.47 (t, J=6.2 Hz, 2H), 4.36-4.29 (m, 3H), 4.17 (ddd, J=12.0, 6.1, 1.9 Hz, 2H), 3.66 (s, 2H), 3.04 (t, J=5.1 Hz, 2H), 2.83 (s, 2H), 2.35 (t, J=7.3 Hz, 3H), 2.08-2.04 (m, 4H), 2.03 (s, 2H), 1.53 (s, 3H), 0.94-0.88 (m, 3H) ppm.

¹⁹F NMR (282 MHz, Methanol-d₄): δ-68.00 (t, J=11.1 Hz).

LC/MS: m/z=906.3 [M+H]⁺ amu.

### Synthesis of Compound 60

Compound 60 was synthesized following the general procedures used for Intermediate 6-1 using the corresponding electrophile, and then following the general procedures used for Compound 57. Compound 60 was obtained as an amorphous off-white solid.

¹H NMR (300 MHz, Methanol-d₄): δ 9.30 (s, 1H), 8.32 (d, J=5.1 Hz, 1H), 7.39 (d, J=8.6 Hz, 1H), 7.21-7.08 (m, 3H), 6.94 (d, J=5.1 Hz, 1H), 6.92-6.86 (m, 2H), 6.85-6.73 (m, 3H), 6.41-6.32 (m, 1H), 5.56 (dd, J=10.1, 3.2 Hz, 1H), 5.02 (t, J=3.9 Hz, 2H), 4.84 (d, J=5.2 Hz, 1H), 4.33 (t, J=4.8 Hz, 2H), 3.99-3.89 (m, 3H), 3.64 (s, 3H), 3.39-3.32 (m, 2H), 3.25 (d, J=8.0 Hz, 3H), 3.10 (q, J=8.4, 6.8 Hz, 3H), 2.83 (s, 3H), 2.44 (dd, J=13.8, 10.2 Hz, 1H), 2.25-2.14 (m, 1H), 2.06-1.84 (m, 4H), 1.74 (s, 3H), 1.51-1.44 (m, 1H), 1.29 (s, 2H) ppm.

¹⁹F NMR (282 MHz, Methanol-d₄): δ-77.42.

LC/MS: m/z=900.2 [M+H]⁺ amu.

### Synthesis of Compound 61

Compound 61 was synthesized following the general procedures used for Intermediate 6-1 using the corresponding electrophile, and then following the general procedures used for Compound 57. Compound 61 was obtained as an amorphous off-white solid.

¹H NMR (300 MHz, Methanol-d₄): δ 9.32 (s, 1H), 8.31 (d, J=5.1 Hz, 1H), 7.39 (d, J=8.6 Hz, 1H), 7.22-7.07 (m, 3H), 6.95 (d, J=5.1 Hz, 1H), 6.89 (d, J=7.9 Hz, 2H), 6.86-6.74 (m, 3H), 6.42-6.34 (m, 1H), 5.56 (dd, J=10.0, 3.3 Hz, 1H), 5.03 (d, J=3.4 Hz, 2H), 4.35 (t, J=4.7 Hz, 2H), 4.08 (t, J=11.9 Hz, 2H), 3.97 (s, 1H), 3.85 (t, J=5.5 Hz, 2H), 3.65 (s, 3H), 3.39-3.33 (m, 2H), 3.18 (d, J=5.2 Hz, 5H), 2.84 (s, 3H), 2.52-2.45 (m, 1H), 2.18-1.97 (m, 3H), 1.75 (s, 5H), 1.29 (d, J=2.4 Hz, 2H) ppm.

¹⁹F NMR (282 MHz, Methanol-d₄): δ-77.53 (s).

LC/MS: m/z=900.3 [M+H]⁺ amu.

### Synthesis of Compound 62

Compound 62 was synthesized following the general procedures used for Intermediate 6-1 using the corresponding electrophile, and then following the general procedures used for Compound 57. Compound 62 was obtained as an amorphous off-white solid.

¹H NMR (300 MHz, Methanol-d₄): δ 9.29 (s, 1H), 8.23 (dd, J=5.7, 3.2 Hz, 1H), 7.27-7.07 (m, 3H), 6.98 (d, J=5.7 Hz, 1H), 6.93-6.83 (m, 3H), 6.80-6.69 (m, 3H), 6.34 (dd, J=7.4, 1.7 Hz, 1H), 5.46 (dd, J=9.6, 3.5 Hz, 1H), 5.13-5.01 (m, 2H), 4.46-4.36 (m, 1H), 4.33-4.16 (m, 2H), 3.76 (t, J=5.0 Hz, 2H), 3.66 (s, 2H), 3.57-3.46 (m, 2H), 3.44-3.32 (m, 2H), 3.28 (dd, J=0.4 Hz, 3H), 3.09 (q, J=4.3, 3.8 Hz, 3H), 2.84 (d, J=4.3 Hz, 3H), 2.65 (s, 2H), 2.50 (dd, J=14.1, 9.6 Hz, 1H), 2.37 (s, 2H), 2.05-1.94 (m, 1H), 1.65 (q, J=5.9 Hz, 2H), 1.53-1.44 (m, 2H), 1.29 (s, 3H), 0.90 (d, J=8.1 Hz, 5H) ppm.

LC/MS: m/z=892.3 [M+H]⁺ amu.

### Synthesis of Compound 63

Compound 63 was synthesized following the general procedures used for Intermediate 6-1 using the corresponding electrophile, and then following the general procedures used for Compound 57. Compound 63 was obtained as an amorphous off-white solid.

¹H NMR (300 MHz, Methanol-d₄): δ 9.33 (s, 1H), 8.30 (d, J=5.2 Hz, 1H), 7.41 (d, J=8.4 Hz, 1H), 7.22-7.09 (m, 3H), 6.97 (d, J=5.3 Hz, 1H), 6.93-6.86 (m, 2H), 6.85-6.72 (m, 3H), 6.47 (s, 1H), 6.37 (d, J=7.5 Hz, 1H), 6.22 (s, 1H), 5.58 (dd, J=10.2, 3.3 Hz, 1H), 5.07 (d, J=3.8 Hz, 2H), 4.42 (d, J=3.9 Hz, 1H), 4.35 (t, J=4.8 Hz, 2H), 4.14 (d, J=10.8 Hz, 3H), 3.66 (s, 3H), 3.59 (t, J=11.9 Hz, 3H), 3.36 (s, 3H), 3.13 (s, 5H), 2.85 (s, 3H), 2.54-2.40 (m, 1H), 1.95 (s, 3H), 1.76 (s, 3H), 1.69 (s, 2H) ppm.

¹⁹F NMR (282 MHz, Methanol-d₄): δ-77.56 (s).

LC/MS: m/z=930.3 [M+H]⁺ amu.

### Synthesis of Compound 104

Compound 104 was synthesized using the general procedures for Compound 57, except that cyclobutyl zinc bromide was used with the the general procedure used for Compound 29 in Step 4 of the synthesis of Intermediate 6-1 instead of the fluorinated alcohol and the procedures used for the synthesis of Intermediate 6-1, and cyclobutyl zinc bromide was used with the general procedures used to synthesize Compound 29 for the last step of the synthesis of Compound 104. Compound 104 was obtained as an off-white solid.

¹H NMR (400 MHz, Methanol-d₄): δ 9.26 (s, 1H), 8.69 (d, J=5.2 Hz, 1H), 7.67 (d, J=5.3 Hz, 1H), 7.27-7.10 (m, 3H), 7.01-6.95 (m, 1H), 6.90 (td, J=7.4, 1.0 Hz, 1H), 6.54 (dd, J=7.5, 1.7 Hz, 1H), 5.55 (dd, J=10.0, 3.3 Hz, 1H), 5.33-5.09 (m, 2H), 4.45-4.27 (m, 2H), 3.90-3.72 (m, 1H), 3.59-3.44 (m, 1H), 3.38-3.32 (m, 1H), 3.28-2.96 (m, 6H), 2.84 (s, 3H), 2.59-2.30 (m, 8H), 2.19-2.02 (m, 2H), 2.01-1.81 (m, 8H) (44 of 47 protons observed).

LC/MS: m/z=783.3 [M+H]⁺ amu.

### Example 7: Synthesis of Intermediate 7-1, Compound 7-1, Compound 7-2, Compound 7-3, Compound 7-4, Compounds 64 Through 68, Compounds 71 Through 74, Compound 112 Through 116, Compound 119, and Compounds 121, 122 and 124 Through 126

### Synthesis of Intermediate 7-1

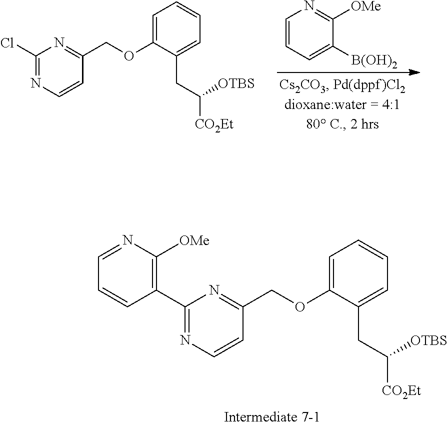 [SMILES: CCOC(=O)[C@H](Cc1ccccc1OCc1ccnc(Cl)n1)O[Si](C)(C)C(C)(C)C ; COc1ncccc1B(O)O ; CCOC(=O)[C@H](Cc1ccccc1OCc1ccnc(-c2cccnc2OC)n1)O[Si](C)(C)C(C)(C)C]

To a solution of ethyl (2S)-2-[tert-butyl(dimethyl)silyl]oxy-3-[2-[(2-chloropyrimidin-4-yl)methoxy]phenyl]propanoate (85 mg, 0.1900 mmol; see steps 1-3 from procedures for synthesis of Intermediate 6-1) in 1,4-Dioxane (6 mL) was added (2-methoxy-3-pyridyl)boronic acid (57.65 mg, 0.3800 mmol), Pd(dppf)Cl₂ (13.79 mg, 0.0200 mmol), Cs₂CO₃ (122.8 mg, 0.3800 mmol), and water (1.5 mL). The resulting mixture was heated at 80° C. under inert atmosphere. After 2 hours, the mixture was cooled to ambient temperature then partitioned between EtOAc (40 mL) and water (20 mL). The aqueous layer was extracted with EtOAc (20 mL), combined organic layers were dried over MgSO₄, filtered, and concentrated, and the crude residue was purified by silica gel chromatography to afford Intermediate 7-1 as a light brown solid.

¹H NMR (300 MHz, CDCl₃): δ 8.95 (d, J=5.1 Hz, 1H), 8.33 (dd, J=5.0, 2.0 Hz, 1H), 8.15 (dd, J=7.4, 2.0 Hz, 1H), 7.73 (d, J=5.0 Hz, 1H), 7.25 (ddd, J=7.6, 6.1, 1.7 Hz, 2H), 7.07 (dd, J=7.4, 5.0 Hz, 1H), 7.01-6.94 (m, 1H), 6.91 (d, J=8.2 Hz, 1H), 5.27 (d, J=1.4 Hz, 2H), 4.57 (dd, J=9.6, 3.8 Hz, 1H), 4.25 (qd, J=7.1, 2.1 Hz, 2H), 4.07 (s, 3H), 3.42 (dd, J=13.2, 3.8 Hz, 1H), 2.93 (dd, J=13.1, 9.6 Hz, 1H), 1.32 (t, J=7.1 Hz, 3H), 0.78 (s, 9H), −0.13 (s, 3H), −0.25 (s, 3H) ppm.

LC/MS: m/z=524.1 [M+H]⁺ amu.

### Synthesis of Compound 64

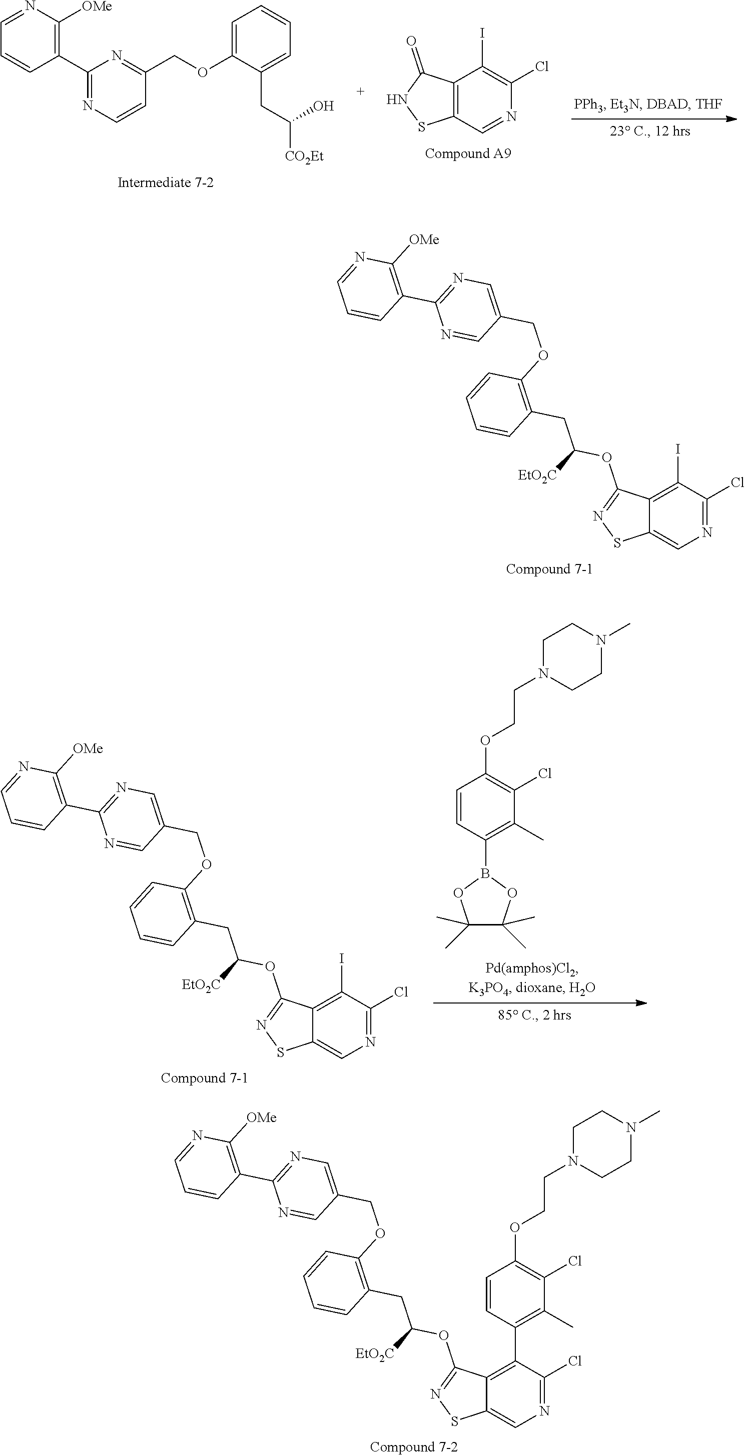

Intermediate 7-2 was synthesized by using Intermediate 7-1 and following Step 5 of the general procedures used to synthesize Intermediate 6-1. Compound 7-1 was synthesized using Compound A9, Intermediate 7-2, and following the general procedures used to synthesize Compound 3-2. Then, Compound 7-2 was synthesized using Compound B-6, Compound 7-1, and following the general procedures used to synthesize Compound 3-3. Compound 64 was synthesized using Compound 7-2 and following the general procedures used to synthesize Compound 35.

¹H NMR (300 MHz, Methanol-d₄): δ 9.30 (s, 1H), 8.86 (d, J=5.3 Hz, 1H), 8.28 (dd, J=5.0, 2.0 Hz, 1H), 8.11 (dd, J=7.4, 2.0 Hz, 1H), 7.81 (d, J=5.3 Hz, 1H), 7.31-6.94 (m, 10H), 6.86 (d, J=8.5 Hz, 1H), 6.77 (dt, J=7.5, 3.3 Hz, 3H), 6.40 (d, J=6.8 Hz, 1H), 5.54-5.41 (m, 1H), 5.27 (d, J=2.3 Hz, 3H), 4.25 (d, J=5.4 Hz, 3H), 3.98 (s, 5H), 3.46-3.32 (m, 10H), 3.26-2.96 (m, 3H), 2.85 (d, J=5.6 Hz, 5H), 2.37 (s, 4H) ppm.

LC/MS: m/z=876.2 [M+H]⁺ amu.

### Synthesis of Compound 65

Compound 65 was synthesized following the general procedures used for Compound 64, wherein the corresponding aryl boronate ester or acid was used when performing the general procedures used for Intermediate 7-1 and the corresponding aryl boronate ester or acid was used when performing the general procedures used for Compound 35. Compound 65 was obtained as an amorphous off-white solid.

¹H NMR (300 MHz, Methanol-d₄): δ 9.31 (s, 1H), 8.87 (d, J=5.1 Hz, 1H), 7.85 (d, J=7.3 Hz, 1H), 7.77 (d, J=5.2 Hz, 1H), 7.58 (d, J=7.4 Hz, 1H), 7.54-7.33 (m, 3H), 7.25-7.07 (m, 3H), 6.98 (d, J=8.2 Hz, 1H), 6.93-6.74 (m, 4H), 6.41 (d, J=7.4 Hz, 1H), 5.61-5.52 (m, 1H), 5.34-5.16 (m, 2H), 4.82 (s, 3H), 4.33 (d, J=5.2 Hz, 2H), 3.64 (s, 3H), 3.41 (d, J=14.5 Hz, 1H), 3.23 (s, 3H), 3.17 (d, J=5.8 Hz, 5H), 2.83 (s, 3H), 1.75 (s, 3H) ppm.

LC/MS: m/z=901.2 [M+H]⁺ amu.

### Synthesis of Compound 66

Compound 66 was synthesized following the general procedures used for Compound 64, wherein the corresponding aryl boronate ester or acid was used when performing the general procedures used for Intermediate 7-1 and the corresponding aryl boronate ester or acid was used when performing the general procedures used for Compound 35. Compound 66 was obtained as an amorphous off-white solid.

¹H NMR (300 MHz, Methanol-d₄): δ 9.31 (s, 1H), 8.88 (d, J=5.2 Hz, 1H), 7.88 (dd, J=7.7, 1.8 Hz, 1H), 7.82 (d, J=5.2 Hz, 1H), 7.56 (td, J=7.8, 1.8 Hz, 1H), 7.47-7.29 (m, 3H), 7.23-7.04 (m, 3H), 7.00-6.77 (m, 6H), 6.41 (dd, J=7.5, 1.7 Hz, 1H), 5.58 (dd, J=10.1, 3.2 Hz, 1H), 5.33-5.19 (m, 2H), 4.33 (t, J=4.7 Hz, 2H), 3.65 (s, 3H), 3.41 (dd, J=14.0, 3.2 Hz, 1H), 3.12 (q, J=6.3, 5.6 Hz, 3H), 2.83 (s, 3H), 1.75 (s, 3H) ppm.

LC/MS: m/z=923.3 [M+H]⁺ amu.

### Synthesis of Compound 67

Compound 67 was synthesized following the general procedures used for Compound 64, wherein the corresponding aryl boronate ester or acid was used when performing the general procedures used for Intermediate 7-1 and the corresponding aryl boronate ester or acid was used when performing the general procedures used for Compound 35. Compound 67 was obtained as an amorphous off-white solid.

¹H NMR (300 MHz, Methanol-d₄): δ 9.31 (s, 1H), 8.87 (d, J=5.3 Hz, 1H), 7.87 (d, J=5.3 Hz, 1H), 7.69-7.51 (m, 2H), 7.49-7.36 (m, 2H), 7.31-7.08 (m, 6H), 6.98 (d, J=8.2 Hz, 1H), 6.93-6.75 (m, 4H), 6.44-6.37 (m, 1H), 5.57 (dd, J=10.2, 3.0 Hz, 1H), 5.36-5.19 (m, 3H), 4.34 (t, J=4.8 Hz, 2H), 3.85 (s, 3H), 3.65 (s, 3H), 3.42 (dd, J=14.1, 3.2 Hz, 1H), 3.21-2.99 (m, 3H), 2.84 (s, 4H), 2.65 (s, 4H), 1.76 (s, 3H) ppm.

LC/MS: m/z=905.3 [M+H]⁺ amu.

### Synthesis of Compound 68

Compound 68 was synthesized following the general procedures used for Compound 64, wherein the corresponding aryl boronate ester or acid was used when performing the general procedures used for Intermediate 7-1 and the corresponding aryl boronate ester or acid was used when performing the general procedures used for Compound 35. Compound 68 was obtained as an amorphous off-white solid.

¹H NMR (300 MHz, Methanol-d₄) δ 9.34 (s, 1H), 8.90 (d, J=5.2 Hz, 1H), 8.18 (d, J=7.6 Hz, 1H), 7.90-7.73 (m, 4H), 7.38 (d, J=8.5 Hz, 1H), 7.23-7.07 (m, 3H), 6.97 (d, J=8.2 Hz, 1H), 6.92-6.78 (m, 5H), 6.45 (d, J=6.2 Hz, 1H), 5.58 (dd, J=9.9, 3.3 Hz, 1H), 5.34-5.20 (m, 2H), 4.34 (t, J=4.7 Hz, 2H), 3.65 (s, 3H), 3.52 (s, 3H), 3.45-3.34 (m, 1H), 3.28-3.07 (m, 3H), 2.84 (s, 3H), 2.53 (dd, J=14.0, 10.0 Hz, 1H), 1.77 (s, 3H).

LC/MS: m/z=935.3 [M+H]⁺ amu.

### Synthesis of Compound 71

Compound 71 was synthesized following the general procedures used for Compound 64, wherein the corresponding heteroaryl boronate ester or acid was used when performing the general procedures used for Intermediate 7-1 and the corresponding aryl boronate ester or acid was used when performing the general procedures used for Compound 35. Compound 71 was obtained as an off-white solid.

¹H NMR (400 MHz, Methanol-d₄): δ 9.30 (s, 1H), 8.91 (d, J=5.1 Hz, 1H), 8.26 (d, J=5.5 Hz, 1H), 7.94 (dd, J=5.4, 1.4 Hz, 1H), 7.85-7.78 (m, 2H), 7.41 (d, J=8.5 Hz, 1H), 7.30-7.21 (m, 1H), 7.21-7.07 (m, 3H), 7.01 (ddd, J=8.4, 4.7, 2.6 Hz, 3H), 6.82 (t, J=7.4 Hz, 1H), 6.40 (dd, J=7.4, 1.7 Hz, 1H), 5.59 (dd, J=10.1, 3.1 Hz, 1H), 5.39-5.23 (m, 2H), 4.42-4.28 (m, 2H), 3.99 (s, 3H), 3.42 (dd, J=13.8, 3.1 Hz, 2H), 3.20-3.05 (m, 8H), 2.85 (s, 3H), 2.49 (dd, J=13.9, 10.2 Hz, 1H), 1.75 (s, 3H), 1.32-1.27 (m, 2H) ppm (43 of 43 protons observed).

LC/MS: m/z=876.3 [M+H]⁺ amu.

### Synthesis of Compound 72

Compound 72 was synthesized following the general procedures used for Compound 64, wherein the corresponding heteroaryl boronate ester or acid was used when performing the general procedures used for Intermediate 7-1 and the corresponding aryl boronate ester or acid was used when performing the general procedures used for Compound 35. Compound 72 was obtained as an off-white solid.

¹H NMR (500 MHz, Methanol-d₄): δ 9.29 (s, 1H), 9.05 (d, J=5.1 Hz, 1H), 8.95 (s, 4H), 7.98 (d, J=5.0 Hz, 1H), 7.43 (d, J=8.5 Hz, 1H), 7.30-7.22 (m, 1H), 7.22-7.14 (m, 2H), 7.10 (d, J=7.8 Hz, 1H), 7.05-6.98 (m, 3H), 6.83 (t, J=7.4 Hz, 1H), 6.36 (dd, J=7.5, 1.7 Hz, 1H), 5.60 (dd, J=10.3, 2.9 Hz, 1H), 5.45-5.25 (m, 2H), 4.45-4.35 (m, 2H), 3.46 (dd, J=13.7, 3.0 Hz, 1H), 3.25-3.12 (m, 5H), 2.86 (s, 3H), 2.47 (dd, J=13.8, 10.3 Hz, 1H), 1.75 (s, 3H) ppm (35 of 41 protons observed).

LC/MS: m/z=846.3 [M+H]⁺ amu.

### Synthesis of Compound 112

Compound 112 was synthesized following the general procedures used for Compound 64, except that the corresponding aryl boronate ester or acid was used when performing the general procedures used for the synthesis of Intermediate 7-1, and in the last step of the synthesis of Compound 112, cyclobutyl zinc bromide was used with the general procedures used to synthesize Compound 29. Compound 112 was obtained as an off-white solid.

¹H NMR (400 MHz, Methanol-d₄) δ 9.35-9.21 (m, 1H), 8.82 (d, J=5.1 Hz, 1H), 8.12-8.08 (m, 1H), 8.01-7.94 (m, 1H), 7.70 (d, J=5.1 Hz, 1H), 7.45-7.35 (m, 1H), 7.35-7.09 (m, 5H), 7.06-7.00 (m, 1H), 6.91 (td, J=7.4, 1.0 Hz, 1H), 6.57 (dd, J=7.5, 1.7 Hz, 1H), 5.58 (dd, J=9.9, 3.4 Hz, 1H), 5.47-5.05 (m, 2H), 4.41-4.24 (m, 2H), 4.08-3.76 (m, 5H), 3.62-3.44 (m, 1H), 3.36 (dd, J=14.0, 3.4 Hz, 2H), 3.29-3.18 (m, 10H), 3.18-2.96 (m, 6H), 2.83 (s, 3H), 2.55-2.43 (m, J=19.1, 12.2, 9.5 Hz, 2H), 2.19-2.02 (m, 2H) (50 of 52 protons observed).

LC/MS: m/z=890.4 [M+H]⁺ amu.

### Synthesis of Compound 113

Compound 113 was synthesized following the general procedures used for Compound 64, except that the corresponding aryl boronate ester or acid was used when performing the general procedures used for the synthesis of Intermediate 7-1, and in the last step of the synthesis of Compound 113, cyclobutyl zinc bromide was used with the general procedures used to synthesize Compound 29. Compound 113 was obtained as an off-white solid.

¹H NMR (400 MHz, Methanol-d₄): δ 9.12-8.99 (m, 1H), 8.75-8.61 (m, 1H), 8.55-8.45 (m, 1H), 7.59-7.47 (m, 1H), 7.39-7.33 (m, 2H), 7.32-7.25 (m, 2H), 7.24-7.19 (m, 1H), 7.15-6.98 (m, 2H), 6.95-6.78 (m, 3H), 6.78-6.60 (m, 3H), 6.52-6.42 (m, 1H), 5.41-5.28 (m, 3H), 5.22-5.06 (m, 2H), 4.57-4.40 (m, 2H), 4.18-4.03 (m, 2H), 3.05-2.74 (m, 9H), 2.66-2.50 (m, 5H), 2.12-2.03 (m, 3H), 1.90-1.85 (m, 1H), 1.82-1.67 (m, 2H) (47 of 50 protons observed).

LC/MS: m/z=912.3 [M+H]⁺ amu.

### Synthesis of Compound 114

Compound 114 was synthesized following the general procedures used for Compound 64, except that the corresponding aryl boronate ester or acid was used when performing the general procedures used for the synthesis of Intermediate 7-1, and in the last step of the synthesis of Compound 114, cyclobutyl zinc bromide was used with the general procedures used to synthesize Compound 29. Compound 114 was obtained as an off-white solid.

¹H NMR (400 MHz, Methanol-d₄): δ 9.23 (s, 1H), 9.20-9.10 (m, 1H), 8.92-8.79 (m, 1H), 8.75 (dd, J=8.7, 2.4 Hz, 1H), 7.69 (d, J=5.1 Hz, 1H), 7.50-7.26 (m, 2H), 7.26-7.05 (m, 4H), 7.05-6.96 (m, 2H), 6.96-6.79 (m, 3H), 6.79-6.65 (m, 2H), 6.55 (dd, J=7.4, 1.7 Hz, 1H), 5.57 (dd, J=10.0, 3.2 Hz, 1H), 5.32-5.23 (m, 3H), 4.34 (q, J=5.6, 5.0 Hz, 1H), 3.81 (s, 4H), 3.13-2.94 (m, 1H), 2.82 (s, 4H), 2.68-2.21 (m, 4H), 2.20-1.74 (m, 8H) (44 of 50 protons observed).

LC/MS: m/z=928.3 [M+H]⁺ amu.

### Synthesis of Compound 115

Compound 115 was synthesized following the general procedures used for Compound 64, except that the corresponding aryl boronate ester or acid was used when performing the general procedures used for the synthesis of Intermediate 7-1, and in the last step of the synthesis of Compound 115, cyclobutyl zinc bromide was used with the general procedures used to synthesize Compound 29. Compound 115 was obtained as an off-white solid.

¹H NMR (400 MHz, Methanol-d₄): δ 9.23 (s, 1H), 8.88 (dd, J=5.1, 4.1 Hz, 1H), 8.54-8.42 (m, 1H), 8.42-8.27 (m, 1H), 7.93 (d, J=8.3 Hz, 1H), 7.75 (d, J=5.1 Hz, 1H), 7.28-7.18 (m, 1H), 7.18-7.10 (m, 2H), 7.04-6.98 (m, 1H), 6.91 (td, J=7.4, 1.0 Hz, 1H), 6.56 (dd, J=7.5, 1.7 Hz, 1H), 5.58 (dd, J=10.0, 3.3 Hz, 1H), 5.42-5.22 (m, 3H), 4.43-4.13 (m, 3H), 3.56-3.46 (m, 2H), 3.37 (d, J=3.3 Hz, 2H), 3.13 (q, J=1.8 Hz, 2H), 3.10-2.94 (m, 4H), 2.82 (s, 4H), 2.72 (s, 3H), 2.58-2.31 (m, 3H), 2.13-2.05 (m, 1H), 1.99-1.87 (m, 9H) (49 of 54 protons observed).

LC/MS: m/z=952.4 [M+H]⁺ amu.

### Synthesis of Compound 116

Compound 116 was synthesized following the general procedures used for Compound 64, except that the corresponding aryl boronate ester or acid was used when performing the general procedures used for the synthesis of Intermediate 7-1, and in the last step of the synthesis of Compound 116, cyclobutyl zinc bromide was used with the general procedures used to synthesize Compound 29. Compound 116 was obtained as an off-white solid.

¹H NMR (400 MHz, Methanol-d₄): δ 9.30-9.19 (m, 1H), 8.89-8.81 (m, 1H), 7.97-7.87 (m, 1H), 7.86-7.76 (m, 1H), 7.71-7.58 (m, 2H), 7.29-7.11 (m, 3H), 7.08-6.86 (m, 2H), 6.71-6.37 (m, 1H), 5.70-5.43 (m, 1H), 5.43-5.19 (m, 2H), 4.46-4.16 (m, 2H), 3.67-3.45 (m, 4H), 3.19-3.00 (m, 8H), 2.89-2.76 (m, 5H), 2.62-2.32 (m, 7H), 2.16-1.93 (m, 3H) (44 of 46 protons observed).

LC/MS: m/z=844.3 [M+H]⁺ amu.

### Synthesis of Compound 119

Compound 119 was synthesized following the general procedures used for Compound 64, except that the corresponding aryl boronate ester or acid was used when performing the general procedures used for the synthesis of Intermediate 7-1, and in the last step of the synthesis of Compound 119, cyclobutyl zinc bromide was used with the general procedures used to synthesize Compound 29. Compound 119 was obtained as a yellow solid.

¹H NMR (400 MHz, Methanol-d₄): δ 9.22 (s, 1H), 8.72 (d, J=5.1 Hz, 1H), 8.29 (d, J=8.8 Hz, 2H), 7.56 (d, J=5.1 Hz, 1H), 7.26-7.17 (m, 1H), 7.17-7.08 (m, 2H), 7.08-6.91 (m, 4H), 6.89 (dt, J=8.0, 1.0 Hz, 1H), 6.58 (dd, J=7.5, 1.7 Hz, 1H), 5.58 (dd, J=9.9, 3.4 Hz, 2H), 5.36-5.05 (m, 4H), 4.32 (ddt, J=14.7, 10.5, 5.2 Hz, 4H), 3.74 (dt, J=14.1, 5.4 Hz, 5H), 3.50-3.45 (m, 1H), 3.13 (q, J=1.6 Hz, 2H), 3.02 (t, J=5.2 Hz, 4H), 2.81 (s, 5H), 2.64-2.22 (m, 3H), 2.17 (s, 3H), 2.00 (s, 2H), 1.95-1.83 (m, 6H) (50 of 55 protons observed).

LC/MS: m/z=931.4 [M+H]⁺ amu.

### Synthesis of Compound 121

Compound 121 was synthesized following the general procedures used for Compound 64, except that the corresponding aryl boronate ester or acid was used when performing the general procedures used for the synthesis of Intermediate 7-1, and in the last step of the synthesis of Compound 121, cyclobutyl zinc bromide was used with the general procedures used to synthesize Compound 29. Compound 121 was obtained as a white solid.

¹H NMR (400 MHz, Methanol-d₄): δ 9.23 (s, 1H), 8.89-8.87 (m, 1H), 8.86 (d, J=5.2 Hz, 1H), 8.39 (ddd, J=7.8, 1.8, 1.2 Hz, 1H), 7.92 (ddd, J=7.7, 1.9, 1.2 Hz, 1H), 7.72 (d, J=5.1 Hz, 1H), 7.66 (d, J=2.3 Hz, 1H), 7.61-7.51 (m, 1H), 7.30-7.19 (m, 1H), 7.13 (q, J=8.5 Hz, 2H), 7.05 (dd, J=8.3, 1.1 Hz, 1H), 6.91 (td, J=7.4, 1.0 Hz, 1H), 6.72 (d, J=2.3 Hz, 1H), 6.59 (dd, J=7.5, 1.7 Hz, 1H), 5.58 (dd, J=10.0, 3.4 Hz, 1H), 5.44-5.20 (m, 3H), 4.45-4.11 (m, 3H), 3.97 (s, 3H), 3.55-3.46 (m, 1H), 3.39-3.33 (m, 2H), 3.13 (q, J=1.6 Hz, 1H), 3.03-2.95 (m, 5H), 2.80 (s, 4H), 2.67-2.29 (m, 4H), 2.11-1.94 (m, 3H), 1.94-1.83 (m, 4H) (49 of 49 protons observed).

LC/MS: m/z=885.3 [M+H]⁺ amu.

### Synthesis of Compound 122

Compound 122 was synthesized following the general procedures used for Compound 64, except that the corresponding aryl boronate ester or acid was used when performing the general procedures used for the synthesis of Intermediate 7-1, and in the last step of the synthesis of Compound 122, cyclobutyl zinc bromide was used with the general procedures used to synthesize Compound 29. Compound 122 was obtained as a white solid.

¹H NMR (400 MHz, Methanol-d₄): δ 9.22 (s, 1H), 8.87 (d, J=5.2 Hz, 1H), 8.52 (d, J=8.8 Hz, 2H), 7.76-7.67 (m, 3H), 7.26-7.18 (m, 1H), 7.18-7.08 (m, 2H), 7.02 (d, J=8.2 Hz, 1H), 6.91 (dt, J=7.2, Hz, 1H), 6.57 (dd, J=7.5, 1.7 Hz, 1H), 5.59 (dd, J=10.0, 3.3 Hz, 1H), 5.50-5.19 (m, 1H), 4.42 (t, J=7.7 Hz, 2H), 4.38-4.27 (m, 2H), 4.23 (t, J=8.0 Hz, 2H), 3.54-3.45 (m, 4H), 3.13 (q, J=1.6 Hz, 1H), 3.04-2.97 (m, 2H), 2.82 (s, 3H), 2.56-2.34 (m, 5H), 2.12-2.01 (m, 1H), 1.99 (s, 3H), 1.88 (s, 6H) (46 of 50 protons observed).

LC/MS: m/z=888.4 [M+H]⁺ amu.

### Synthesis of Compound 73

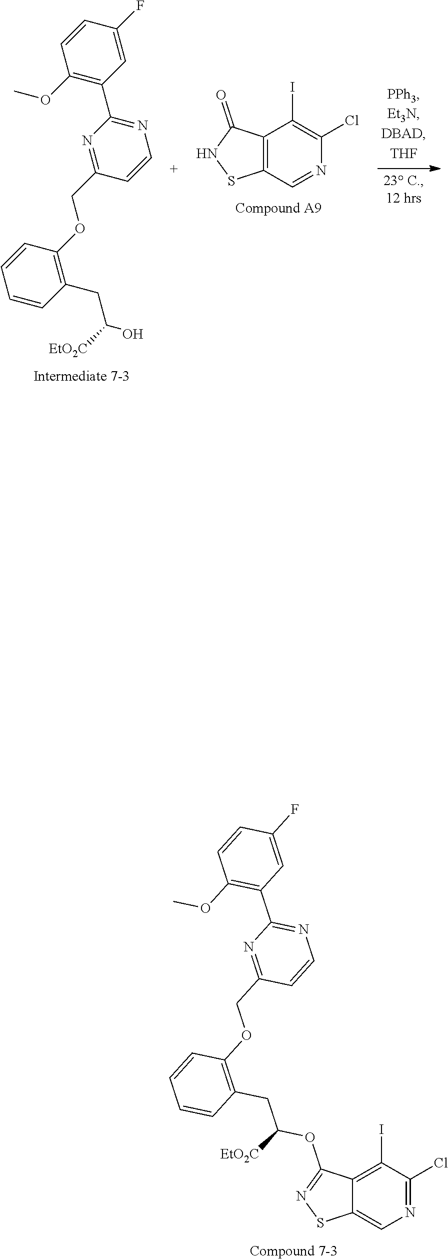 [SMILES: CCOC(=O)[C@@H](O)Cc1ccccc1OCc1ccnc(-c2cc(F)ccc2OC)n1 ; O=c1[nH]sc2cnc(Cl)c(I)c12 ; CCOC(=O)[C@@H](Cc1ccccc1OCc1ccnc(-c2cc(F)ccc2OC)n1)Oc1nsc2cnc(Cl)c(I)c12]

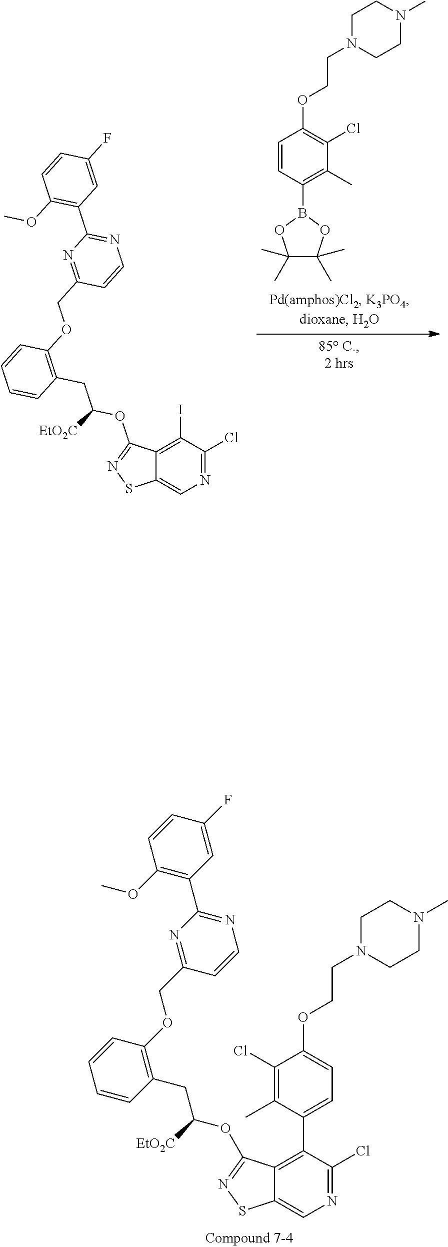

Intermediate 7-3 was synthesized by first following the general procedures used to synthesize Intermediate 7-1 and using the corresponding aryl boronate ester or acid, and then following Step 5 of the general procedures used to synthesize Intermediate 6-1. Compound 7-3 was synthesized using Compound A9, Intermediate 7-3, and following the general procedures used to synthesize Compound 3-2. Then, Compound 7-4 was synthesized by using Compound B-6, Compound 7-3 and following the general procedures used to synthesize Compound 3-3. Compound 73 was synthesized using Compound 7-4 and following the general procedures used to synthesize Compound 29.

¹H NMR (400 MHz, Methanol-d₄): δ 9.26 (s, 1H), 8.86 (s, 1H), 7.81 (d, J=5.2 Hz, 1H), 7.43-7.36 (m, 1H), 7.33-7.11 (m, 5H), 7.11-7.02 (m, 2H), 7.01-6.95 (m, 1H), 6.95-6.90 (m, 1H), 6.84 (td, J=7.5, 1.0 Hz, 1H), 6.50 (dd, J=7.5, 1.7 Hz, 1H), 5.47 (dd, J=9.5, 3.8 Hz, 1H), 5.34-5.19 (m, 2H), 4.43-4.28 (m, 2H), 3.84 (s, 3H), 3.67-3.53 (m, 1H), 3.37 (dd, J=14.1, 3.8 Hz, 1H), 3.24-3.14 (m, 4H), 2.85 (s, 4H), 2.72-2.64 (m, 1H), 2.59-2.43 (m, 2H), 2.43-2.24 (m, 2H), 2.18 (s, 3H), 2.16-1.97 (m, 2H), 1.97-1.78 (m, 3H) ppm (46 of 46 protons observed).

LC/MS: m/z=853.3 [M+H]⁺ amu.

### Synthesis of Compound 74

Compound 74 was synthesized following the general procedures used to synthesize Compound 73 and using the corresponding heteroaryl boronate ester or acid when performing the general procedures used to synthesize Intermediate 7-1. Compound 74 was obtained as an off-white solid.

¹H NMR (400 MHz, Methanol-d₄): δ 9.24 (s, 1H), 9.04 (d, J=5.1 Hz, 1H), 8.94 (s, 4H), 7.98 (d, J=5.2, 1H), 7.27-7.18 (m, 1H), 7.17 (s, 2H), 7.06-7.01 (m, 1H), 6.93 (td, J=7.4, 1.0 Hz, 1H), 6.53 (dd, J=7.5, 1.7 Hz, 1H), 5.60 (dd, J=10.2, 3.0 Hz, 1H), 5.45-5.31 (m, 2H), 4.46-4.35 (m, 2H), 3.57-3.45 (m, 1H), 3.40 (dd, J=13.8, 3.0 Hz, 1H), 3.21-3.10 (m, 4H), 2.86 (s, 3H), 2.59-2.28 (m, 3H), 1.99-1.82 (m, 5H) ppm (35 of 44 protons observed).

LC/MS: m/z=806.3 [M+H]⁺ amu.

### Synthesis of Intermediate 7-4

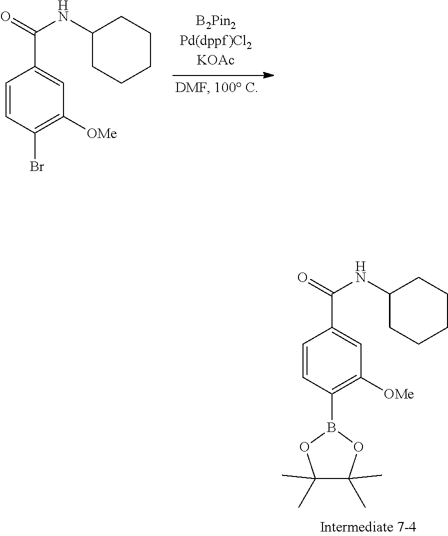 [SMILES: COc1cc(C(=O)NC2CCCCC2)ccc1Br ; COc1cc(C(=O)NC2CCCCC2)ccc1B1OC(C)(C)C(C)(C)O1]

A vial containing 4-bromo-N-cyclohexyl-3-methoxy-benzamide (300 mg, 0.960 mmol), bis(pinacolato)diboron (293 mg, 1.15 mmol), [1,1′-bis(diphenylphosphino)ferrocene]dichloropalladium(II) (39.3 mg, 0.050 mmol), and potassium acetate (283 mg, 2.88 mmol) was evacuated and backfilled 3× with nitrogen. The vial was degassed and dimethylformamide (5.65 mL) was added and heated to 100° C. for 12 hours. The reaction was cooled and filtered over a 2-inch plug of celite. The organics were transferred to a separatory funnel and backextracted with water (30 mL, 3 times), dried over sodium sulfate, filtered, and concentrated in vacuo. The crude reaction mixture was purified via flash chromatography (0 to 60% ethyl acetate in hexanes). Fractions containing the Intermediate 7-4 were pooled and concentrated in vacuo to yield a clear oil.

LC/MS: m/z=360.2 [M+H]⁺ amu.

### Synthesis of Compound 124

Compound 124 was synthesized following the general procedures used to synthesize Compound 73, except that the corresponding intermediate for Compound 124 (i.e., instead of Intermediate 7-3) was synthesized by first using Intermediate 7-4 and following the general procedures used to synthesize Intermediate 7-1, and then following Step 5 of the general procedures used to synthesize Intermediate 6-1. Compound 124 was obtained as a white solid.

¹H NMR (400 MHz, Methanol-d₄): δ 9.23 (s, 1H), 8.85 (d, J=5.2 Hz, 1H), 7.82 (d, J=5.3 Hz, 1H), 7.69 (d, J=7.9 Hz, 1H), 7.59 (d, J=1.6 Hz, 1H), 7.50 (dd, J=7.9, 1.6 Hz, 1H), 7.26-7.18 (m, 1H), 7.18-7.07 (m, 2H), 7.00 (d, J=8.1 Hz, 1H), 6.91 (td, J=7.4, 1.0 Hz, 1H), 6.57 (dd, J=7.5, 1.7 Hz, 1H), 5.56 (dd, J=9.9, 3.2 Hz, 1H), 5.46-5.18 (m, 2H), 4.47-4.20 (m, J=4.4 Hz, 2H), 3.91 (s, 4H), 3.57-3.43 (m, 3H), 3.41-3.34 (m, 1H), 3.13 (q, J=1.6 Hz, 1H), 3.04 (t, J=4.9 Hz, 2H), 2.82 (s, 3H), 2.59-2.35 (m, 3H), 2.09-1.93 (m, 6H), 1.93-1.78 (m, 8H) (47 of 58 protons observed).

LC/MS: m/z=960.5 [M+H]⁺ amu.

### Synthesis of Compound 125

Compound 125 was synthesized following the general procedures used to synthesize Compound 124 and using 1-(4-(6-bromopyridin-3-yl)piperazin-1-yl)ethan-1-one when performing the general procedures used to synthesize Intermediate 7-4. Compound 125 was obtained as an off-white solid.

¹H NMR (400 MHz, Methanol-d₄): δ 9.22 (s, 1H), 9.05-8.97 (m, 1H), 8.79 (d, J=5.3 Hz, 1H), 8.72-8.49 (m, 1H), 7.72-7.61 (m, 1H), 7.21 (ddd, J=8.2, 7.5, 1.7 Hz, 1H), 7.18-7.06 (m, 3H), 7.06-6.97 (m, 1H), 6.91 (td, J=7.4, 1.0 Hz, 1H), 6.55 (dd, J=7.5, 1.7 Hz, 1H), 5.58 (dd, J=10.0, 3.2 Hz, 1H), 5.37-5.14 (m, 2H), 4.40-4.25 (m, 2H), 3.88-3.64 (m, 9H), 3.54-3.45 (m, 1H), 3.39-3.34 (m, 2H), 3.13 (q, J=1.6 Hz, 1H), 3.10-3.04 (m, 2H), 2.83 (s, 3H), 2.61-2.22 (m, 3H), 2.18 (s, 3H), 2.15-2.00 (m, 1H), 1.90 (s, 6H) (48 of 54 protons observed).

LC/MS: m/z=932.4 [M+H]⁺ amu.

### Synthesis of Compound 126

Compound 126 was synthesized following the general procedures used to synthesize Compound 124 and using 14-(2-(4-bromophenoxy)ethyl)morpholine when performing the general procedures used to synthesize Intermediate 7-4. Compound 126 was obtained as an off-white solid.

¹H NMR (400 MHz, Methanol-d₄): δ 9.23 (s, 1H), 8.77 (d, J=5.1 Hz, 1H), 8.56-8.34 (m, 2H), 7.67 (d, J=5.1 Hz, 1H), 7.24-7.18 (m, 2H), 7.18-7.08 (m, 8H), 7.00 (dd, J=8.3, 1.0 Hz, 1H), 6.91 (td, J=7.5, 1.0 Hz, 1H), 6.54 (dd, J=7.4, 1.7 Hz, 1H), 5.59 (dd, J=10.0, 3.2 Hz, 1H), 5.44-5.12 (m, 2H), 4.53-4.43 (m, 2H), 4.36 (q, J=4.9, 4.4 Hz, 2H), 4.09-3.76 (m, 2H), 3.75-3.66 (m, 2H), 3.66-3.38 (m, 3H), 3.15-3.04 (m, 4H), 2.84 (s, 3H), 2.65-2.31 (m, 3H), 2.15-2.00 (m, 1H), 2.00-1.81 (m, 7H) (50 of 56 protons observed).

LC/MS: m/z=934.4 [M+H]⁺ amu.

### Example 8: Synthesis of Intermediate 8-1, and Compounds 69 and 70

### Synthesis of Intermediate 8-1

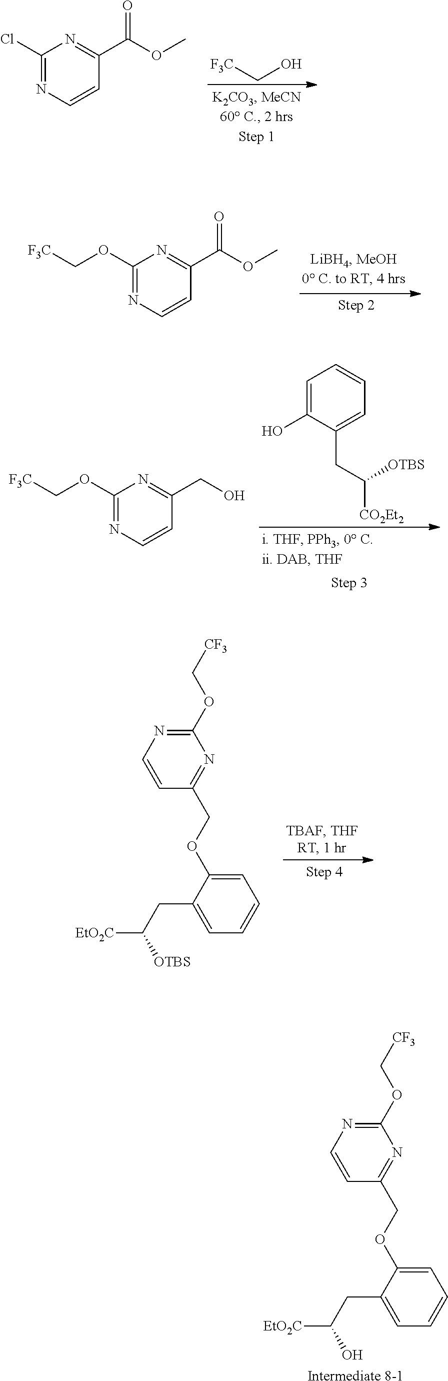

### Step 1

To a solution of methyl 2-chloropyrimidine-4-carboxylate (500 mg, 2.9 mmol) in MeCN (20 mL) was added 2,2,2-trifluoroethanol (5 mL, 69.54 mmol) and K₂CO₃ (800.83 mg, 5.79 mmol). The resulting mixture was heated at 60° C. under N₂ for 2 hours. The mixture was cooled, quenched with water (5 mL), and partitioned between EtOAc (50 mL) and water (20 mL). The aqueous layer was extracted with EtOAc (30 mL). The combined organic layers were dried over MgSO₄, filtered and concentrated.

¹H NMR (300 MHz, CDCl₃): δ 8.81 (d, J=4.9 Hz, 1H), 7.76 (d, J=4.9 Hz, 1H), 4.90 (dd, J=8.2, 6.4 Hz, 2H), 4.04 (s, 3H) ppm. ¹⁹F NMR (282 MHz, CDCl₃): δ-73.70 (td, J=8.4, 4.2 Hz).

LC/MS: m/z=237.2 [M+H]⁺ amu.

### Step 2

To an oven-dry 100 mL flask was added methyl 2-(2,2,2-trifluoroethoxy)pyrimidine-4-carboxylate (300 mg, 1.27 mmol) and methanol (8 mL). The resulting mixture was cooled to 0° C. followed by slow addition of LiBH₄ (0.7 mL, 1.4 mmol). After the addition, the ice bath was remove and the reaction warmed to ambient temperature. Upon completion, the reaction was quenched with water (2 mL) and the solvent removed under reduced pressure. The resulting residue was partitioned between EtOAc (20 mL) and water (10 mL) and extracted with 20% iPrOH in CHCl₃ (2×10 mL). The combined organic layers were dried over MgSO₄, filtered, and concentrated. The crude product was purified by silica gel chromatography to afford the desired product as a yellow solid.

¹H NMR (300 MHz, CDCl₃): δ 8.53 (d, J=5.0 Hz, 1H), 7.13 (d, J=5.0 Hz, 1H), 4.85 (q, J=8.3 Hz, 2H), 4.77 (s, 2H) ppm. ¹⁹F NMR (282 MHz, CDCl₃): δ-73.76 (t, J=8.3 Hz).

LC/MS: m/z=209.3 [M+H]⁺ amu.

### Step 3

To a solution of ethyl (2S)-2-[tert-butyl(dimethyl)silyl]oxy-3-(2-hydroxyphenyl)propanoate (205 mg, 0.6300 mmol) in THF (5 mL) was added [2-(2,2,2-trifluoroethoxy)pyrimidin-4-yl]methanol (170.94 mg, 0.8200 mmol) and PPh₃ (331.41 mg, 1.26 mmol). The resulting mixture was cooled to 0° C., and DBAD (290.61 mg, 1.26 mmol) added in a single portion. Reaction mixture was quenched with water (2 mL) and partitioned between EtOAc (40 mL) and water (20 mL). The aqueous layer was extracted with more EtOAc (30 mL). The combined organic layer was dried over MgSO₄, filtered, and concentrated. The crude product was purified by silica gel chromatography to afford the desired product as a white solid.

¹H NMR (500 MHz, CDCl₃): δ 8.61 (d, J=5.0 Hz, 1H), 7.51 (d, J=5.0 Hz, 1H), 7.24 (t, J=7.3 Hz, 2H), 6.97 (td, J=7.3, 1.0 Hz, 1H), 6.87-6.83 (m, 1H), 5.14 (d, J=2.8 Hz, 2H), 4.86 (q, J=8.4 Hz, 2H), 4.56-4.53 (m, 1H), 4.24 (qd, J=7.2, 5.3 Hz, 2H), 3.38 (dd, J=13.2, 3.9 Hz, 1H), 2.93 (dd, J=13.1, 9.6 Hz, 1H), 1.31 (t, J=7.1 Hz, 3H), 0.78 (s, 9H), −0.13 (s, 3H), −0.24 (s, 3H) ppm. ¹⁹F NMR (282 MHz, CDCl₃): δ-73.76 (d, J=16.4 Hz).

LC/MS: m/z=515.3 [M+H]⁺ amu.

### Step 4

To a solution of ethyl (2S)-2-[tert-butyl(dimethyl)silyl]oxy-3-[2-[[2-(2,2,2-trifluoroethoxy)pyrimidin-4-yl]methoxy]phenyl]propanoate (183 mg, 0.3600 mmol) in THE (5 mL) was added TBAF (0.6 mL, 0.6000 mmol). The resulting mixture was stirred for an hour, and the mixture was concentrated onto silica gel. The crude material was purified by silica gel chromatography to afford Intermediate 8-1 as a brown oil.

¹H NMR (300 MHz, CDCl₃): δ 8.59 (d, J=5.0 Hz, 1H), 7.40 (dd, J=5.1, 0.8 Hz, 1H), 7.27-7.21 (m, 2H), 7.03-6.96 (m, 1H), 6.84 (d, J=8.2 Hz, 1H), 5.16 (s, 2H), 4.86 (q, J=8.3 Hz, 2H), 4.56 (dd, J=8.2, 4.7 Hz, 1H), 4.31-4.17 (m, 2H), 3.33 (dd, J=13.7, 4.7 Hz, 1H), 3.04 (dd, J=13.7, 8.2 Hz, 1H), 1.28 (t, J=7.2 Hz, 3H) ppm.

¹⁹F NMR (282 MHz, CDCl₃): δ-73.76 (d, J=16.4 Hz).

LC/MS: m/z=401.2 [M+H]⁺ amu.

### Synthesis of Compound 69

Compound 69 was synthesized using Intermediate 8-1 and following the general procedures used for Compound 64.

¹H NMR (500 MHz, Methanol-d₄): δ 9.35 (s, 1H), 8.68 (d, J=5.1 Hz, 1H), 7.63 (d, J=5.1 Hz, 1H), 7.48 (d, J=3.5 Hz, 1H), 7.31 (q, J=7.5 Hz, 1H), 7.19 (tt, J=15.5, 7.3 Hz, 3H), 7.06 (d, J=9.1 Hz, 2H), 6.99 (d, J=8.2 Hz, 1H), 6.86 (t, J=7.3 Hz, 1H), 6.46-6.35 (m, 1H), 5.61 (dd, J=10.3, 3.0 Hz, 1H), 5.23 (q, J=15.1 Hz, 2H), 5.08-5.00 (m, 4H), 4.90 (s, OH), 4.38 (dq, J=10.6, 5.3 Hz, 1H), 3.52-3.40 (m, 1H), 3.11 (d, J=14.8 Hz, 2H), 2.89 (s, 3H), 2.52 (dd, J=13.8, 10.4 Hz, 1H), 1.79 (s, 3H) ppm.

LC/MS: m/z=867.2 [M+H]⁺ amu.

### Synthesis of Compound 70

Compound 70 was synthesized following the general procedures used for Compound 69, wherein the corresponding electrophile was used when performing the general procedures used for Intermediate 8-1 and the corresponding aryl boronate ester or acid was used when performing the general procedures used for Compound 35. Compound 70 was obtained as an amorphous off-white solid.

¹H NMR (300 MHz, Methanol-d₄) δ 9.32 (s, 1H), 8.27 (d, J=5.6 Hz, 1H), 7.40 (d, J=8.5 Hz, 1H), 7.15 (q, J=8.2 Hz, 3H), 7.02 (d, J=5.6 Hz, 1H), 6.92-6.73 (m, 4H), 6.36 (dd, J=7.5, 1.6 Hz, 1H), 5.56 (dd, J=10.1, 3.2 Hz, 1H), 5.16-5.01 (m, 2H), 4.36 (s, 2H), 4.25-4.06 (m, 2H), 3.65 (s, 3H), 3.58-3.44 (m, 3H), 3.37 (s, 3H), 3.33 (s, OH), 3.26-3.10 (m, OH), 2.85 (d, J=1.0 Hz, 3H), 2.45 (dd, J=13.9, 10.2 Hz, 1H), 1.95 (d, J=20.4 Hz, 2H), 1.75 (s, 3H), 1.56 (dd, J=9.5, 4.6 Hz, 1H).

LC/MS: m/z=894.3 [M+H]⁺ amu.

### Example 9: Synthesis of Intermediate 9-1, Intermediate 9-2, Compound 9-1 and Compounds 92 and 93

### Synthesis of Intermediate 9-1

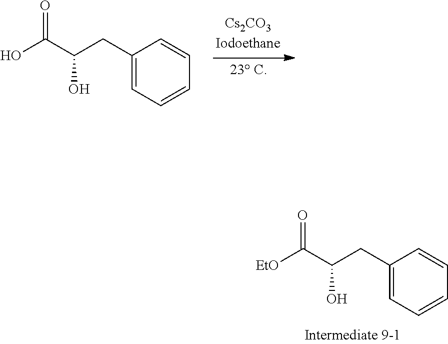 [SMILES: O=C(O)[C@@H](O)Cc1ccccc1 ; CCOC(=O)[C@@H](O)Cc1ccccc1]

A round-bottom flask was charged with (2S)-2-hydroxy-3-phenyl-propanoic acid (1.0 g, 6.02 mmol) and dissolved in N,N-dimethylformamide. Then, cesium carbonate (2.0 g, 6.14 mmol) was slowly added and stirred at 23° C. until the evolution of gas ceased. Iodoethane (0.97 mL, 12.04 mmol) was added and the mixture was stirred at 23° C. for 18 hours.

The reaction was stopped and quenched with the addition of deionized water (20 mL) and poured into a separatory funnel. The organic phase was separated and the aqueous phase was washed with ethyl acetate (20 mL, 2 times). The combined organics were concentrated onto silica gel. Silica gel chromatography was performed (0-20% ethyl acetate/hexanes). The product fractions were pooled and concentrated to yield Intermediate 9-1 as a white solid.

LC/MS: m/z=195.1 [M+H]⁺ amu.

### Synthesis of Intermediate 9-2

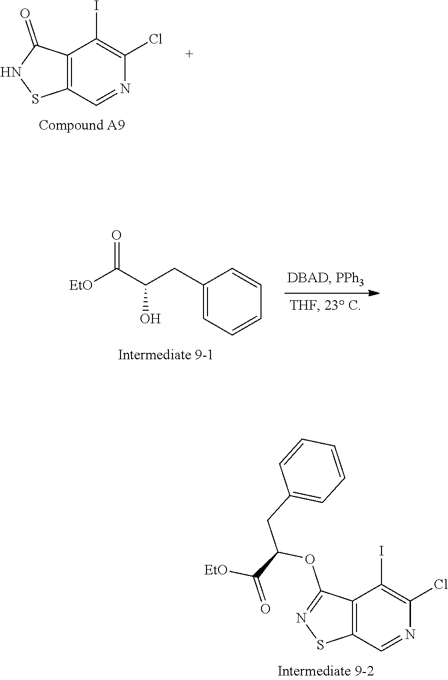 [SMILES: O=c1[nH]sc2cnc(Cl)c(I)c12 ; CCOC(=O)[C@@H](O)Cc1ccccc1 ; CCOC(=O)[C@@H](Cc1ccccc1)Oc1nsc2cnc(Cl)c(I)c12]

To a suspension of Compound A9 (100 mg, 0.32 mmol), Intermediate 9-1 (65 mg, 0.34 mmol), and triphenylphosphine (126 mg, 0.48 mmol) in tetrahydrofuran (3.2 mL) was added di-tert-butyl azodicarboxylate (110 mg, 0.48 mmol) as a solid in one portion. The reaction was stirred at ambient temperature for 10 hours, at which time LC/MS analysis showed complete conversion to the desired product.

The reaction was concentrated onto silica gel. Silica gel chromatography was performed with refractive index detection (0-50% ethyl acetate/hexanes). The product fractions were pooled and concentrated to yield Intermediate 9-2 as a yellow solid.

LC/MS: m/z=489.0 [M+H]⁺ amu.

### Synthesis of Compound 9-1

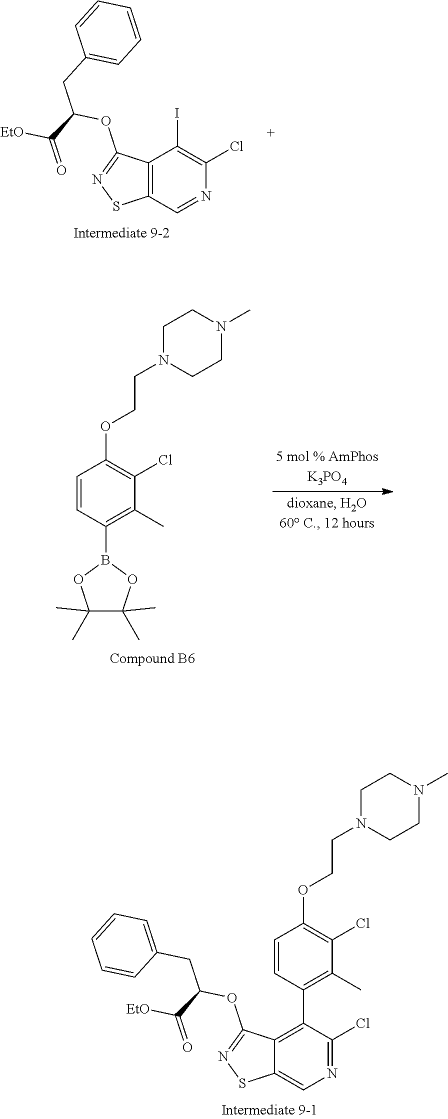

A flask containing Intermediate 9-2 (0.050 mg, 0.102 mmol) was charged with Compound B6 (0.048 g, 0.123 mmol), bis(di-tert-butyl(4-dimethylaminophenyl)phosphine)dichloropalladium(II) (3.6 mg, 0.005 mmol), and potassium phosphate tribasic (0.065 g, 0.307 mmol). The solids were dissolved in degassed 1,4-dioxane (0.33 mL) and deionized water (0.17 mL). The reaction was stirred at 80° C. for 2 hours, allowed to cool and poured into a separatory funnel containing water (3 mL). The organic phase was separated and the aqueous phase was washed with dichloromethane (5 mL, 2 times). The combined organic extracts were concentrated onto silica gel. Silica gel chromatography was performed (0-20% methanol/dichloromethane). The product fractions were pooled and concentrated to yield Compound 9-1 as a yellow solid and a mixture of diastereomers.

LC/MS: m/z=629.2 [M+H]⁺ amu.

### Synthesis of Compound 92

Compound 92 was synthesized using Compound 9-1, the corresponding aryl boronate ester or acid, and following the general procedures used for Compound 7, with the exception that Na₂CO₃ was used instead of K₃PO₄. Compound 92 was obtained as an off-white solid.

¹H NMR (500 MHz, Methanol-d₄): δ 9.31 (s, 1H), 7.40 (d, J=8.5 Hz, 1H), 7.29-7.21 (m, 1H), 7.20-7.07 (m, 5H), 7.04-6.97 (m, 2H), 6.75 (d, J=6.9 Hz, 2H), 5.36 (dd, J=10.2, 2.7 Hz, 1H), 4.37 (qt, J=10.6, 4.5 Hz, 2H), 3.27-3.11 (t, J=5.0 Hz, 3H), 3.07 (dd, J=14.3, 2.8 Hz, 1H), 2.86 (s, 3H), 2.59 (dd, J=14.3, 10.2 Hz, 1H), 1.78 (s, 3H) ppm (26 of 34 protons observed).

LC/MS: m/z=661.2 [M+H]⁺ amu.

### Synthesis of Compound 93

Ethyl (S)-2-hydroxy-3-(2-methoxyphenyl)propanoate and Compound A9 were used as starting materials when performing the procedure for the synthesis of Intermediate 9-2; the resulting product and Compound B6 were used when performing the procedure for the synthesis of Compound 9-1; and lastly, the resulting product was used when performing the general procedures used for Compound 92 to produce Compound 93. Compound 93 was obtained as an off-white solid.

¹H NMR (500 MHz, Methanol-d₄): δ 9.29 (s, 1H), 7.37 (d, J=8.5 Hz, 1H), 7.24 (ddd, J=9.0, 7.7, 5.8 Hz, 1H), 7.17-7.07 (m, 3H), 7.04-6.97 (m, 2H), 6.87 (d, J=8.2 Hz, 1H), 6.72 (t, J=7.4 Hz, 1H), 6.30 (dd, J=7.4, 1.7 Hz, 1H), 5.40 (dd, J=9.9, 3.6 Hz, 1H), 4.40-4.29 (m, 2H), 3.83 (s, 3H), 3.24 (s, 1H), 3.19 (dd, J=13.8, 3.6 Hz, 2H), 3.10 (t, J=5.0 Hz, 3H), 2.82 (s, 3H), 2.38 (dd, J=13.8, 10.0 Hz, 1H), 1.75 (s, 3H) ppm (30 of 36 protons observed).

LC/MS: m/z=691.2 [M+H]⁺ amu.

### Example 10: Synthesis of Compounds 94 Through 101

### Synthesis Compound 94

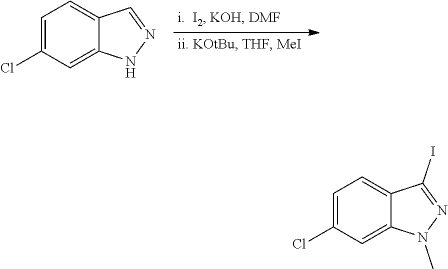

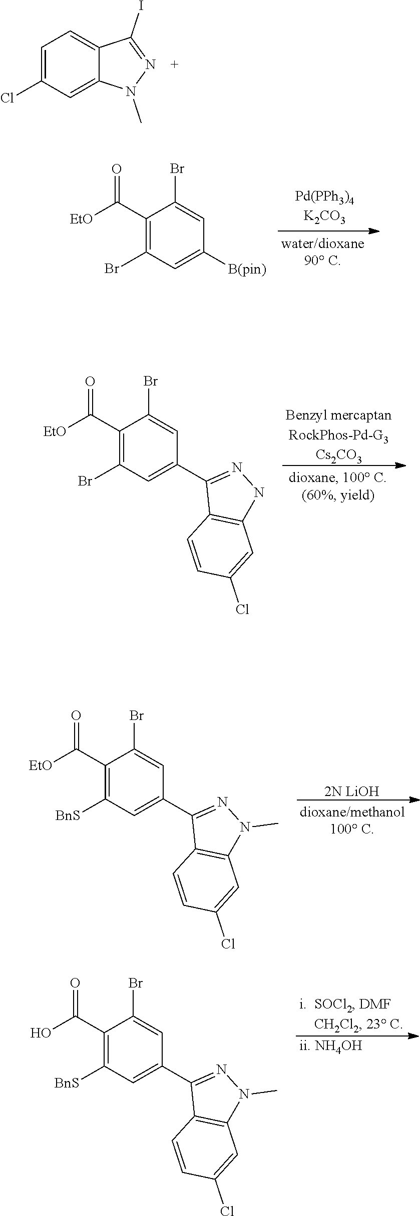

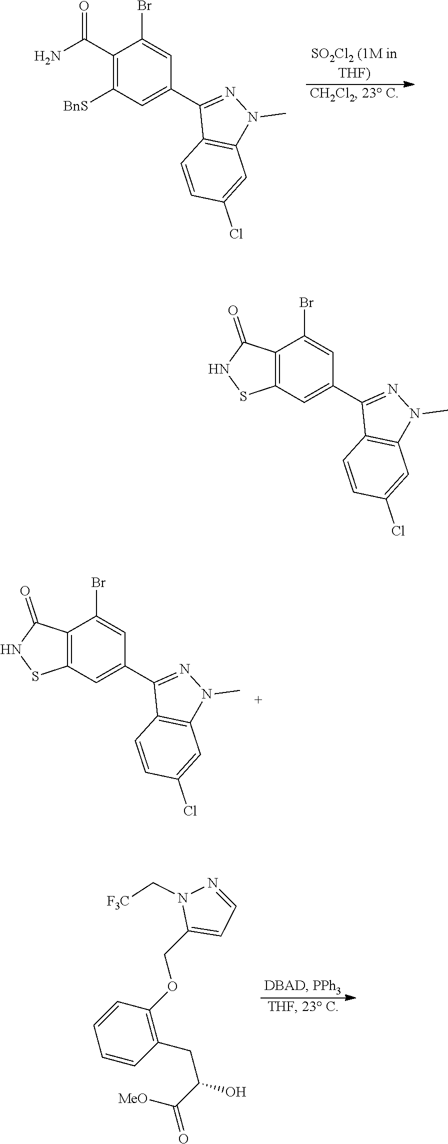

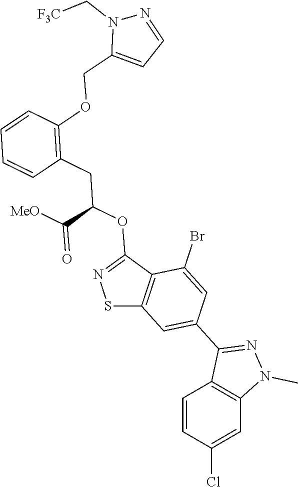 [SMILES: COC(=O)[C@@H](Cc1ccccc1OCc1ccnn1CC(F)(F)F)Oc1nsc2cc(-c3nn(C)c4cc(Cl)ccc34)cc(Br)c12]

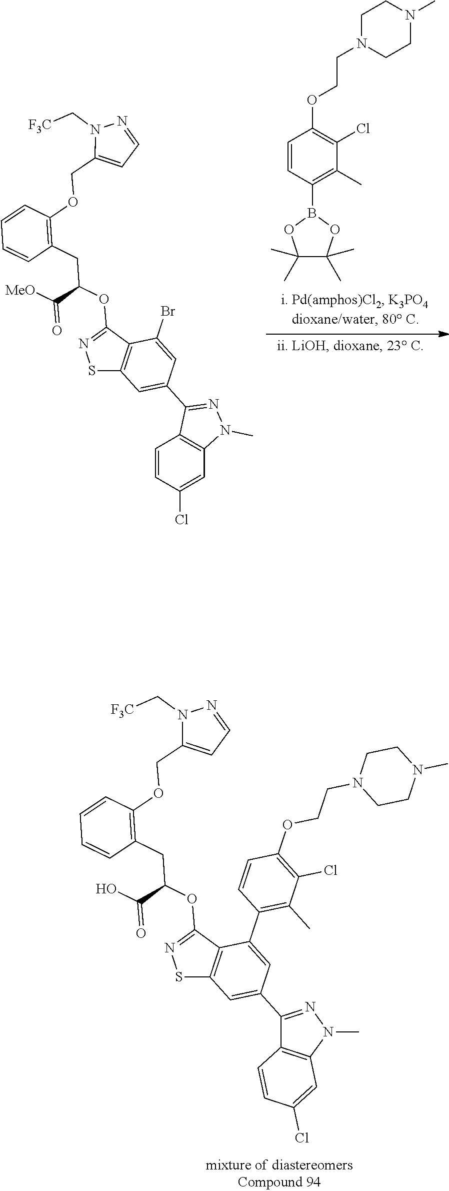

Compound 94: ¹H NMR (400 MHz, Methanol-d₄): δ 8.45 (s, 1H), 8.14-7.99 (m, 1H), 7.82-7.65 (m, 2H), 7.63-7.45 (m, 1H), 7.42-6.93 (m, 6H), 6.91-6.64 (m, 1.3H), 6.66-6.39 (m, 1.6H), 6.34-6.18 (m, 0.6H), 5.39 (dd, J=9.6, 3.8 Hz, 0.6H), 5.32-5.15 (m, 3.2H), 5.15-4.96 (m, 3H), 4.46-4.24 (m, 2H), 4.12 (s, 3H), 3.16-2.94 (m, 6.4H), 2.84 (s, 3.7H), 2.55-2.38 (m, 1H), 2.34 (s, 2H), 2.11 (s, 1.2H), 1.63-0.98 (m, 7H) ppm (mixture of diastereomers).

LC/MS: m/z=908.2 [M+H]⁺ amu.

### Synthesis Compound 95

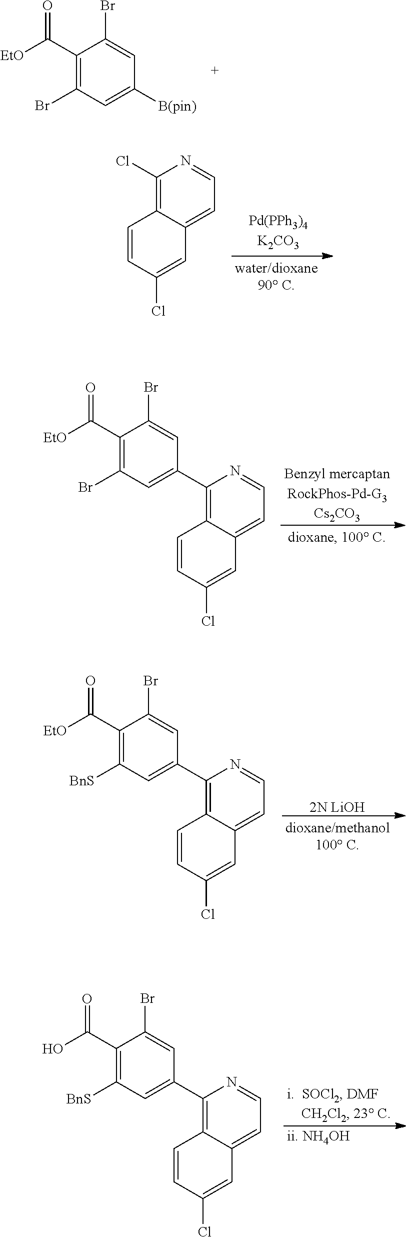

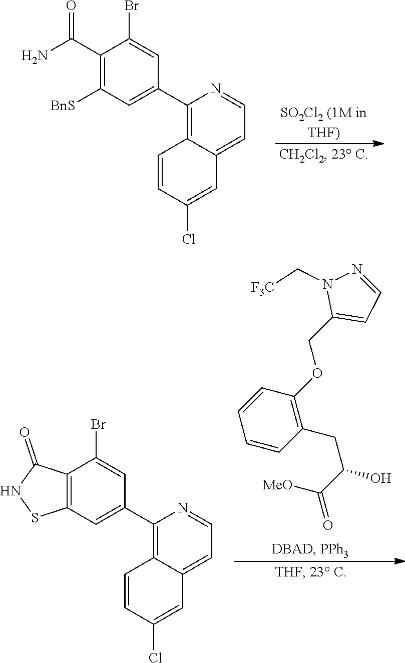

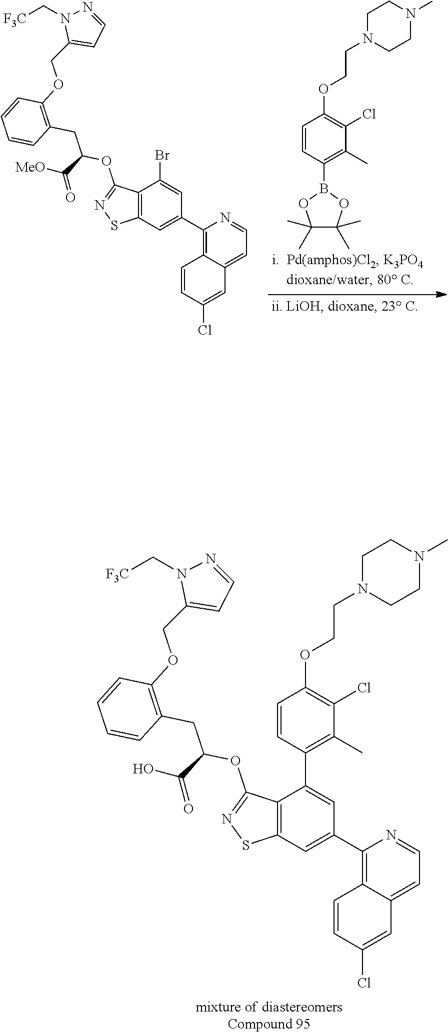

Compound 95: ¹H NMR (400 MHz, Methanol-d₄): δ 8.54-8.44 (m, 1H), 8.19-7.96 (m, 3H), 7.88 (d, J=5.8 Hz, 1H), 7.62 (ddd, J=9.1, 3.2, 2.1 Hz, 1H), 7.51-7.45 (m, 1.3H), 7.39-7.26 (m, 1.7H), 7.16-7.05 (m, 2H), 7.05-6.89 (m, 3H), 6.81-6.63 (m, 1.5H), 6.47 (dd, J=7.2, 1.9 Hz, 1.2H), 6.40-6.32 (m, 0.6H), 6.19 (dd, J=7.5, 1.7 Hz, 0.6H), 6.10 (dd, J=4.4, 2.9 Hz, 0.5H), 5.33 (dd, J=9.6, 3.7 Hz, 2H), 5.25-5.09 (m, 3.2H), 5.06-4.48 (m, 3H), 4.34-4.13 (m, 2.4H), 3.61-3.51 (m, 1H), 3.16-2.84 (m, 12H), 2.74-2.68 (m, 4H), 2.56 (s, 1H), 2.47-2.34 (m, 1H), 2.27 (s, 2H), 2.03 (s, 1H), 1.43-1.37 (m, 5H) ppm (mixture of diastereomers).

LC/MS: m/z=905.2 [M+H]⁺ amu.

### Synthesis Compound 96

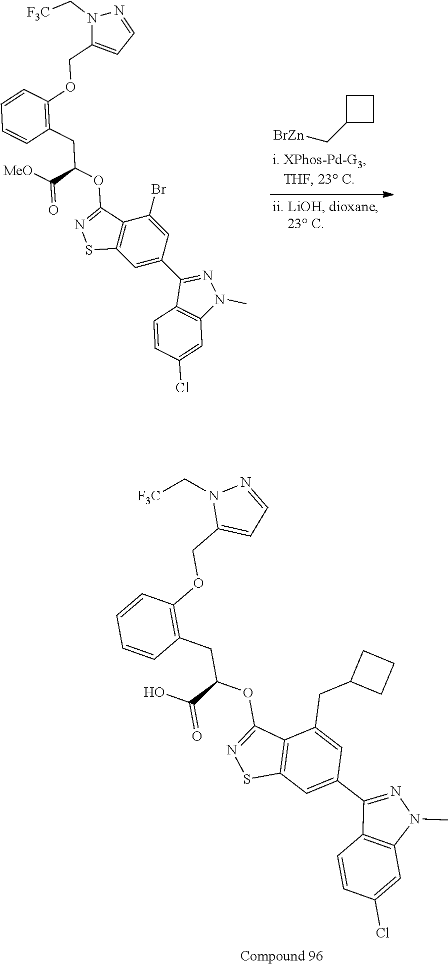

Compound 96 was synthesized following the general procedures used for the synthesis of Compound 94, except that the last cross coupling reaction was performed using cyclobutylmethylzinc bromide and following the general procedures used for the final cross coupling reaction used for Compound 29.

¹H NMR (500 MHz, Methanol-d₄): δ 8.14 (d, J=1.4 Hz, 1H), 8.00 (d, J=8.6 Hz, 1H), 7.68-7.63 (m, 2H), 7.55 (d, J=1.9 Hz, 1H), 7.32 (d, J=7.4 Hz, 1H), 7.31-7.26 (m, 1H), 7.23 (dd, J=6.8, 1.2, 1H), 7.12 (d, J=8.2 Hz, 1H), 7.03-6.94 (m, 1H), 6.60-6.51 (m, 1H), 5.75 (dd, J=9.2, 4.3 Hz, 1H), 5.26 (s, 2H), 5.05 (qd, J=8.6, 2.9 Hz, 2H), 4.10 (s, 3H), 3.53 (dd, J=14.1, 4.3 Hz, 1H), 3.22 (dd, J=14.1, 9.3 Hz, 1H), 3.11 (dd, J=13.5, 8.0 Hz, 1H), 2.90-2.70 (m, 1H), 2.10-1.67 (m, 7H), 1.32-1.22 (m, 1H) ppm (32 of 32 protons observed).

LC/MS: m/z=710.2 [M+H]⁺ amu.

### Synthesis Compound 97

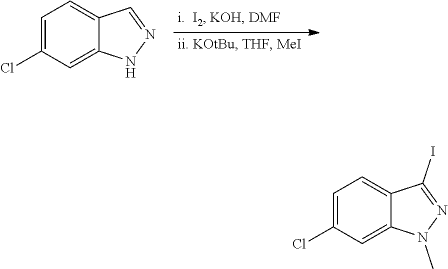

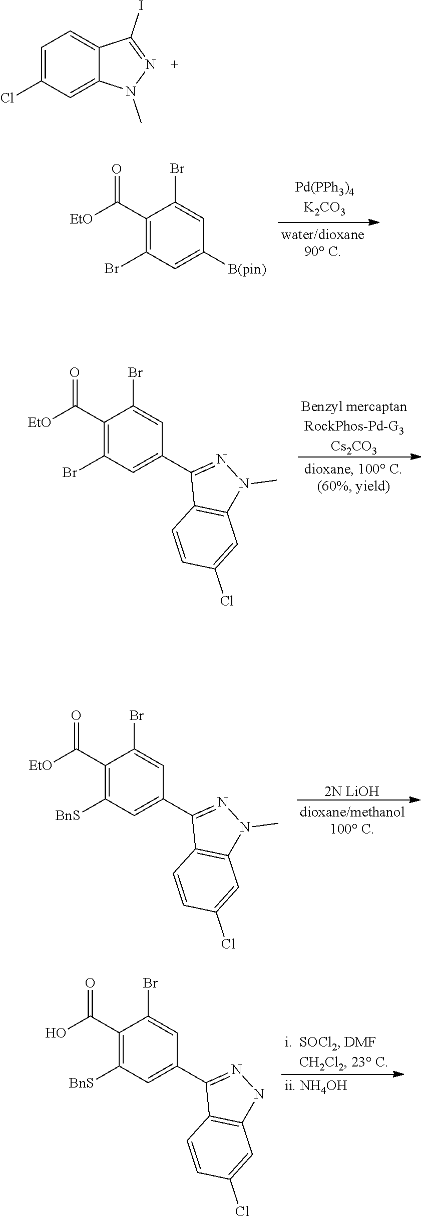

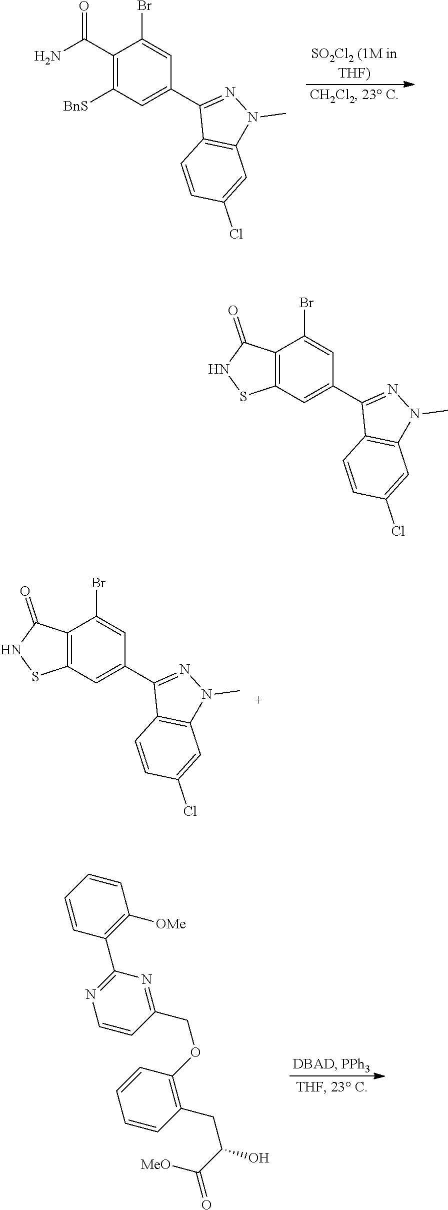

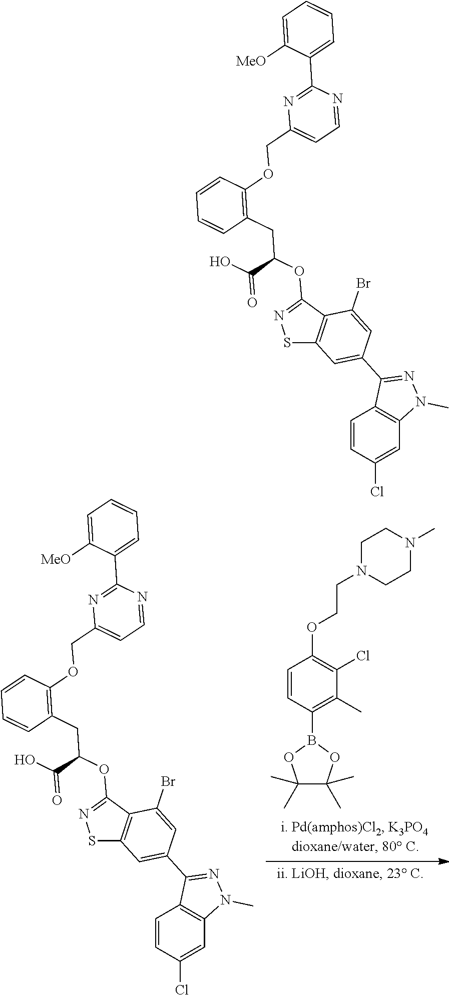

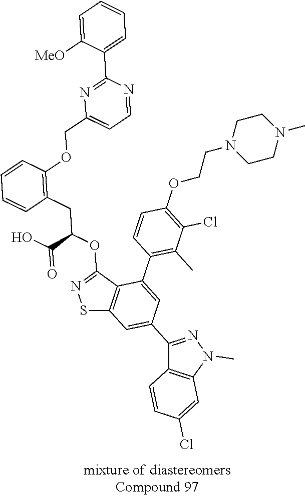 [SMILES: COc1ccccc1-c1nccc(COc2ccccc2C[C@@H](Oc2nsc3cc(-c4nn(C)c5cc(Cl)ccc45)cc(-c4ccc(OCCN5CCN(C)CC5)c(Cl)c4C)c23)C(=O)O)n1]

Compound 97: ¹H NMR (500 MHz, Methanol-d₄): δ 8.89-8.78 (m, 1H), 8.49-8.32 (m, 1H), 8.04 (dd, J=8.8, 6.4 Hz, 1H), 8.12-7.98 (m, 1H), 7.94-7.83 (m, 1H), 7.83-7.61 (m, 3H), 7.60-7.44 (m, 1H), 7.43-7.34 (m, 0.4H), 7.28-7.03 (m, 5.5H), 6.97 (t, J=7.3 Hz, 1H), 6.87 (t, J=7.2, 0.5H), 6.80 (t, J=7.4 Hz, 0.5H), 6.44-6.26 (m, 1H), 5.55 (dd, J=8.0, 2.8, 0.3H), 5.44 (dd, J=9.7, 3.4 Hz, 0.5H), 5.38-5.18 (m, 2H), 4.47-4.29 (m, 1.8H), 4.13-4.10 (m, 3H), 3.94-3.82 (m, 3H), 3.51-3.38 (m, 1H), 3.24-2.98 (m, 3H), 2.85 (s, 3H), 2.75-2.51 (m, 1H), 2.35 (s, 2H), 2.14 (s, 1H) ppm (mixture of diastereomers).

LC/MS: m/z=710.2 [M+H]⁺ amu.

### Synthesis of Compound 98

Compound 98 was synthesized with the general procedures used for Compound 94 and using 5-chloro-2-methyl-1H-benzo[d]imidazole as the starting material. Compound 98 was obtained as an amorphous off-white solid.

¹H NMR (400 MHz, Acetonitrile-d₃): δ 7.96 (dd, J=1.8, 1.2 Hz, 1H), 7.69 (d, J=1.5 Hz, 1H), 7.45 (dd, J=8.5, 1.8 Hz, 1H), 7.25-7.05 (m, 5H), 7.05-6.85 (m, 3H), 6.80-6.66 (m, 1H), 6.44-6.23 (m, 2H), 5.28 (ddd, J=22.7, 9.3, 3.9 Hz, 1H), 5.06 (d, J=11.5 Hz, 2H), 4.95-4.78 (m, 2H), 4.22 (ddd, J=18.8, 10.5, 4.7 Hz, 2H), 3.20-2.96 (m, 4H), 2.62 (d, J=3.9 Hz, 3H), 2.21 (s, 1H), 1.87 (d, J=2.5 Hz, 2H) ppm.

LC/MS: m/z=875.3 [M+H]⁺ amu.

### Synthesis of Compound 99

Compound 99 was synthesized with the general procedures used for Compound 94 and using 6-chloro-1-methyl-1,3-dihydro-2H-benzo[d]imidazol-2-one as the starting material. Compound 99 was obtained as an amorphous off-white solid.

LC/MS: m/z=924.8 [M+H]⁺ amu.

### Synthesis of Compound 100

Compound 100 was synthesized with the general procedures used for Compound 94 and using 3,5-dichloro-2-methyl-1H-pyrrolo[2,3-b]pyridine as the starting material. Compound 100 was obtained as an amorphous off-white solid.

LC/MS: m/z=943.3 [M+H]⁺ amu.

### Synthesis of Compound 101

Compound 101 was synthesized with the general procedures used for Compound 96 and using 3,5-dichloro-2-methyl-1H-pyrrolo[2,3-b]pyridine as the starting material. Compound 101 was obtained as an amorphous off-white solid.

¹H NMR (400 MHz, Chloroform-d): δ 8.13 (d, J=2.3 Hz, 1H), 7.86 (d, J=2.3 Hz, 1H), 7.57 (dd, J=15.6, 1.8 Hz, 2H), 7.36-7.28 (m, 2H), 6.98 (ddd, J=7.4, 4.6, 1.2 Hz, 3H), 6.41 (d, J=1.9 Hz, 1H), 5.71 (dd, J=9.4, 4.0 Hz, 1H), 5.16 (d, J=1.5 Hz, 2H), 4.99 (p, J=8.4 Hz, 2H), 3.52 (dd, J=14.3, 4.0 Hz, 1H), 3.30 (dd, J=14.2, 7.1 Hz, 1H), 3.21 (dd, J=14.4, 9.4 Hz, 1H), 3.13 (dd, J=14.2, 7.8 Hz, 1H), 2.74 (q, J=7.7 Hz, 1H), 2.31 (s, 3H), 2.02 (ddt, J=23.3, 13.6, 5.7 Hz, 2H), 1.92-1.66 (m, 4H) ppm.

LC/MS: m/z=744.6 [M+H]⁺ amu.

### Example 11: Synthesis of Intermediate 11-1, Compound 109, Compound 117, Compound 118 and Compound 123

### Synthesis of Intermediate 11-1

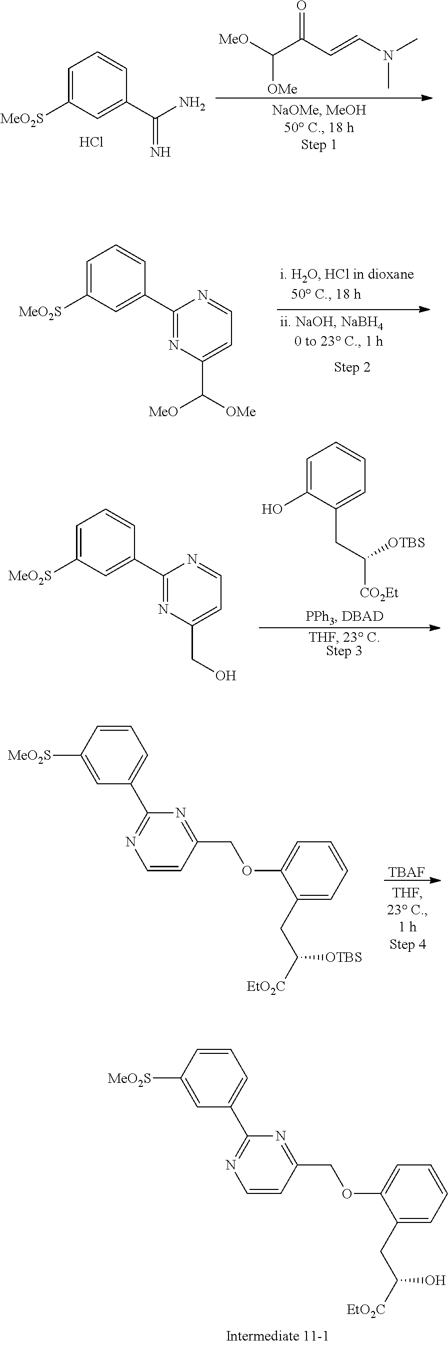

### Step 1

To a solution of a 3-methylsulfonylbenzamidine;hydrochloride (100.0 mg, 0.430 mmol) and (E)-4-(dimethylamino)-1,1-dimethoxy-but-3-en-2-one (66.4 mg, 0.380 mmol) in Methanol (6 mL) at room temperature was added NaOMe (1.3 mL, 0.650 mmol) dropwise via an addition funnel. After the addition, the resulting mixture was heated at 50° C. under N₂ for 3 hours. The reaction mixture was cooled to room temperature and concentrated in vacuo. The residue was partitioned between EtOAc (40 mL) and water (20 mL). The aqueous layer was extracted with more EtOAc (20 mL). The combined organic layers were dried over MgSO₄, filtered, and concentrated in vacuo. The crude product was purified by the silica gel chromatography to yield 4-(dimethoxymethyl)-2-(3-(methylsulfonyl)phenyl)pyrimidine as a brown solid (40 mg, 30% yield).

¹H NMR (300 MHz, CDCl₃): δ 9.10 (t, J=1.8 Hz, 1H), 8.90 (dd, J=5.1, 1.2 Hz, 1H), 8.83-8.77 (m, 1H), 8.09 (dt, J=7.8, 1.6 Hz, 1H), 7.72 (dd, J=8.2, 7.5 Hz, 1H), 7.53 (d, J=5.1 Hz, 1H), 5.38 (d, J=1.1 Hz, 1H), 3.51 (d, J=0.5 Hz, 6H), 3.15 (t, J=0.7 Hz, 3H).

LC/MS: m/z=308.2 [M+H]⁺ amu.

### Step 2

To a suspension of 4-(dimethoxymethyl)-2-(3-methylsulfonylphenyl)pyrimidine (130.0 mg, 0.420 mmol) in water (3 mL) was added hydrogen chloride (4 M in dioxane) (0.85 mL, 3.38 mmol; in dioxane). The resulting mixture was capped and heated at 50° C. for 18 hours.

The reaction mixture was cooled to 0° C. followed by addition of sodium hydroxide (145.6 mg, 3.64 mmol) until pH ˜8-9; then NaBH₄ (32.5 mg, 0.860 mmol) was added and the reaction mixture was warmed and continued to stir at room temperature for 45 minutes. The reaction was quenched with the addition of water (3 mL) and then partitioned between EtOAc (40 mL) and water (20 mL). The aqueous layer was extracted with 20% iPrOH in CHC₃ (30 mL, 2 times). The combined organic layers were dried over MgSO₄, filtered, and concentrated in vacuo. The crude product was purified by silica gel chromatography to yield (2-(3-(methylsulfonyl)phenyl)pyrimidin-4-yl)methanol as a light brown paste (87.0 mg, 78% yield).

¹H NMR (300 MHz, CDCl₃): δ 9.08 (td, J=1.8, 0.5 Hz, 1H), 8.84 (s, 1H), 8.81-8.77 (m, 1H), 8.10 (ddd, J=7.8, 2.0, 1.2 Hz, 1H), 7.74 (td, J=7.8, 0.5 Hz, 1H), 7.33 (dt, J=5.1, 0.7 Hz, 1H), 4.88 (d, J=0.7 Hz, 2H), 3.16 (s, 4H) (11 of 12 protons observed).

LC/MS: m/z=264.3 [M+H]⁺ amu.

### Step 3

To a solution of ethyl (2S)-2-[tert-butyl(dimethyl)silyl]oxy-3-(2-hydroxyphenyl)propanoate (78.0 mg, 0.240 mmol) in THE (3 mL) was added [2-(3-methylsulfonylphenyl)pyrimidin-4-yl]methanol (84.0 mg, 0.320 mmol) and triphenylphosphine (127.0 mg, 0.480 mmol). After the resulting mixture became a homogeneous solution, di-tert-butyl azodicarboxylate (111.0 mg, 0.480 mmol) was added and the reaction mixture was continued to stir at room temperature under N₂ for 2.5 hours. The reaction mixture was stopped and purified by silica gel chromatography to yield ethyl (S)-2-((tert-butyldimethylsilyl)oxy)-3-(2-((2-(3-(methylsulfonyl)phenyl)pyrimidin-4-yl)methoxy)phenyl)propanoate as a white solid (101.0 mg, 74% yield).

¹H NMR (300 MHz, CDCl₃): δ 9.12 (s, 1H), 8.91 (d, J=5.1 Hz, 1H), 8.82 (d, J=7.9 Hz, 1H), 8.11 (d, J=7.7 Hz, 1H), 7.75 (q, J=6.8, 5.7 Hz, 2H), 7.01-6.87 (m, 2H), 6.21 (s, 3H), 4.56 (dd, J=9.6, 3.7 Hz, 1H), 4.25 (td, J=8.2, 7.7, 5.6 Hz, 2H), 3.45-3.34 (m, 1H), 3.17 (d, J=1.5 Hz, 3H), 2.99-2.89 (m, 1H), 1.32 (t, J=7.2 Hz, 3H), 0.78 (d, J=1.6 Hz, 10H), −0.13 (s, 3H), −0.24 (s, 3H).

LC/MS: m/z=571.1 [M+H]⁺ amu.

### Step 4

To a solution of ethyl (2S)-2-[tert-butyl(dimethyl)silyl]oxy-3-[2-[[2-(3-methylsulfonylphenyl)pyrimidin-4-yl]methoxy]phenyl]propanoate (101.0 mg, 0.180 mmol) in THE (3 mL) was added TBAF (1.0 M in THF, 0.270 mL, 0.270 mmol). After addition, the resulting mixture was continued to stir at room temperature under N₂ for 1 hour. The reaction was stopped and purified via silica gel chromatography to yield ethyl (S)-2-hydroxy-3-(2-((2-(3-(methylsulfonyl)phenyl)pyrimidin-4-yl)methoxy)phenyl)propanoate as a white solid (72.0 mg, 89% yield).

¹H NMR (300 MHz, CDCl₃): δ 9.12 (s, 1H), 8.89 (d, J=5.1 Hz, 1H), 8.82 (d, J=8.0 Hz, 1H), 8.11 (d, J=7.7 Hz, 1H), 7.75 (t, J=7.9 Hz, 1H), 7.63 (d, J=5.0 Hz, 1H), 7.27 (s, 2H), 7.01 (t, J=7.6 Hz, 1H), 6.91 (d, J=8.5 Hz, 1H), 5.31 (s, 2H), 4.60 (dd, J=8.2, 4.6 Hz, 1H), 4.24 (t, J=7.1 Hz, 2H), 3.37 (dd, J=13.7, 4.5 Hz, 1H), 3.17 (s, 3H), 3.08 (dd, J=13.6, 8.1 Hz, 1H), 1.28 (d, J=2.3 Hz, 3H) (23 of 24 protons observed).

LC/MS: m/z=457.2 [M+H]⁺ amu.

### Synthesis of Compound 118

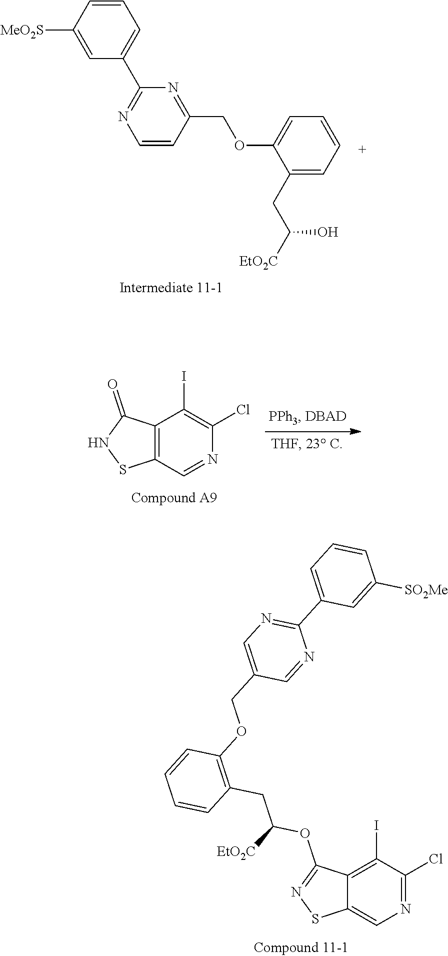 [SMILES: CCOC(=O)[C@@H](O)Cc1ccccc1OCc1ccnc(-c2cccc(S(C)(=O)=O)c2)n1 ; O=c1[nH]sc2cnc(Cl)c(I)c12 ; CCOC(=O)[C@@H](Cc1ccccc1OCc1cnc(-c2cccc(S(C)(=O)=O)c2)nc1)Oc1nsc2cnc(Cl)c(I)c12]

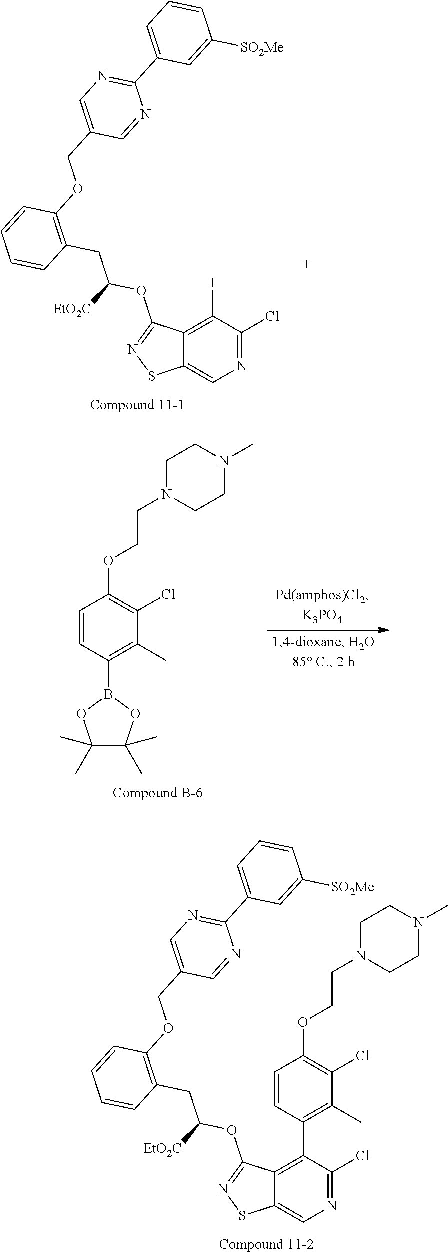

Compound 11-1 was synthesized using compound A9, Intermediate 11-1, and following the general procedure used to synthesized Compound 3-2. Compound 11-2 was synthesized using Compound B-6, Compound 11-1, and following the general procedures used to synthesize Compound 3-3. Compound 118 was synthesized using Compound 11-2, cyclobutyl zinc bromide and following the general procedures used for the synthesis of Compound 29. Compound 118 was obtained as an off-white solid.

¹H NMR (300 MHz, Methanol-d₄): δ 9.24 (s, 1H), 9.03 (s, 1H), 8.91 (d, J=5.2 Hz, 1H), 8.81 (d, J=7.7 Hz, 1H), 8.11 (d, J=7.8 Hz, 1H), 7.78 (t, J=7.1 Hz, 2H), 7.23 (t, J=8.1 Hz, 1H), 7.15 (d, J=2.6 Hz, 2H), 7.04 (d, J=8.3 Hz, 1H), 6.92 (t, J=7.5 Hz, 1H), 6.57 (d, J=7.3 Hz, 1H), 5.58 (d, J=7.8 Hz, 1H), 5.35 (d, J=4.7 Hz, 2H), 3.58-3.46 (m, 1H), 3.40 (s, 2H), 3.24 (d, J=7.1 Hz, 3H), 3.20 (s, 7H), 3.05 (d, J=1.4 Hz, 4H), 2.83 (s, 3H), 2.56-2.46 (m, 2H), 1.89 (s, 3H), 1.33 (d, J=7.3 Hz, 3H) (46 of 47 protons observed).

LC/MS: m/z=883.2 [M+H]⁺ amu.

### Synthesis of Compound 117

Compound 117 was synthesized following the general procedures used for Compound 118, wherein the corresponding amidine was used when performing the general procedures used for the synthesis of Intermediate 11-1. Compound 117 was obtained as an off-white solid.

¹H NMR (400 MHz, Methanol-d₄): δ 9.22 (s, 1H), 8.87 (d, J=5.1 Hz, 1H), 8.47 (dt, J=7.9, 1.2 Hz, 1H), 8.36-8.33 (m, 1H), 7.76 (d, J=5.1 Hz, 1H), 7.60 (t, J=8.0 Hz, 1H), 7.46-7.39 (m, 1H), 7.27-7.17 (m, 1H), 7.17-7.09 (m, 2H), 7.03 (dd, J=8.4, 1.1 Hz, 1H), 6.91 (td, J=7.4, 1.0 Hz, 1H), 6.56 (dd, J=7.5, 1.7 Hz, 1H), 5.57 (dd, J=10.0, 3.2 Hz, 1H), 5.47-5.17 (m, 3H), 4.33 (dq, J=10.0, 5.9 Hz, 3H), 3.58-3.43 (m, 2H), 3.38 (dd, J=13.2, 3.3 Hz, 2H), 3.13 (q, J=1.6 Hz, 2H), 3.06-2.94 (m, 4H), 2.81 (s, 4H), 2.63-2.23 (m, 4H), 2.13-2.01 (m, 1H), 1.95-1.88 (m, 6H) (41 of 44 protons observed).

LC/MS: m/z=889.3 [M+H]⁺ amu.

### Synthesis of Compound 123

Compound 123 was synthesized following the general procedures used for Compound 118, wherein the corresponding amidine was used when performing the general procedures used for the synthesis of Intermediate 11-1. Compound 123 was obtained as a yellow solid.

¹H NMR (400 MHz, Methanol-d₄): δ 9.14 (s, 1H), 8.62 (d, J=5.1 Hz, 1H), 8.19 (d, J=9.2 Hz, 2H), 7.46 (d, J=5.1 Hz, 1H), 7.17-6.98 (m, 3H), 6.91 (d, J=8.8 Hz, 3H), 6.80 (td, J=7.5, 1.0 Hz, 1H), 6.48 (dd, J=7.5, 1.7 Hz, 1H), 5.57-5.45 (m, 1H), 5.28-5.03 (m, 2H), 4.22 (dq, J=9.4, 5.4 Hz, 2H), 3.75 (t, J=4.9 Hz, 5H), 3.55 (d, J=6.8 Hz, 1H), 3.49-3.33 (m, 2H), 3.17-2.90 (m, 9H), 2.71 (s, 3H), 2.49-2.20 (m, 3H), 2.12-1.91 (m, 2H), 1.85-1.67 (m, 7H) (50 of 52 protons observed).

LC/MS: m/z=890.4 [M+H]⁺ amu.

### Synthesis of Intermediate 11-2

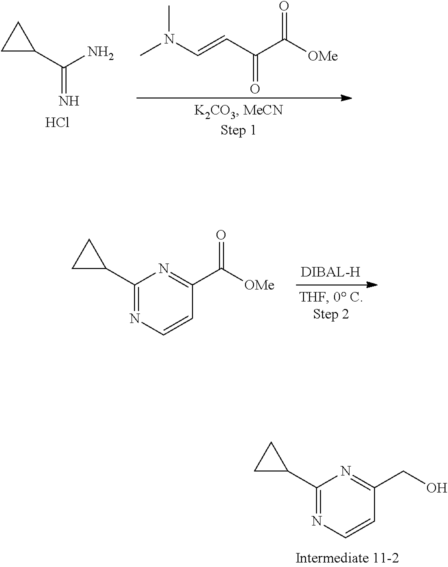

### Step 1

To a solution of cyclopropanecarboximidamide hydrochloride (3.07 g, 25.45 mmol, 0.8 eq, HCl) and methyl (E)-4-(dimethylamino)-2-oxo-but-3-enoate (5 g, 31.81 mmol, 1 eq) in MeCN (100 mL) was added K₂CO₃ (8.79 g, 63.63 mmol, 2.00 eq). The mixture was stirred at 80° C. for 2 hours. The reaction was quenched by addition of H₂O (120 mL) and extracted with ethyl acetate (100 mL, 2 times). The combined organic phase was washed with brine (150 mL), dried over anhydrous Na₂SO₄, filtered and concentrated in vacuo. The residue was purified by silica gel chromatography to yield methyl 2-cyclopropylpyrimidine-4-carboxylate (1.14 g, 6.40 mmol, 20.11% yield) as a pale-yellow oil.

### Step 2

To a solution of methyl 2-cyclopropylpyrimidine-4-carboxylate (1.8 g, 10.10 mmol, 1.00 eq) in THE (30 mL) was added DIBAL-H (1 M, 20.20 mL, 2.00 eq) at 0° C. The mixture was stirred at 0° C. for 1 hour. The reaction was quenched slowly with H₂O (100 mL), then filtered via celite pad and the filtrate was extracted with EtOAc (100 mL, 3 times). The combined organic phase was concentrated to give Intermediate 11-2 (1.00 g, 6.39 mmol, 63.3% yield, 96% purity) as a light-yellow oil.

LC/MS: m/z=151.0 [M+H]⁺ amu.

### Synthesis of Intermediate 109

Compound 109 was synthesized following the general procedures used for Compound 118, wherein Intermediate 11-2 was used instead of Intermediate 11-1. Compound 109 was obtained as an off-white solid.

¹H NMR (400 MHz, Methanol-d₄): δ 9.28 (s, 1H), 8.58 (d, J=5.3 Hz, 1H), 7.59 (d, J=5.2 Hz, 1H), 7.31-7.05 (m, 3H), 7.05-6.80 (m, 2H), 6.53 (dd, J=7.4, 1.7 Hz, 1H), 5.54 (dd, J=10.0, 3.3 Hz, 1H), 5.22-5.03 (m, 2H), 4.53-4.26 (m, 2H), 3.63-3.45 (m, 1H), 3.42-3.35 (m, 2H), 3.25-3.00 (m, 6H), 2.86 (s, 3H), 2.60-2.29 (m, 3H), 2.23 (tt, J=7.7, 5.2 Hz, 1H), 2.18-2.02 (m, 1H), 2.02-1.74 (m, 7H), 1.16-1.08 (m, 5H) (43 of 45 protons observed).

LC/MS: m/z=769.3 [M+H]⁺ amu.

### Example 12: Synthesis of Intermediate 12-1, Compound 110 and Compound 111

### Synthesis of Intermediate 12-1

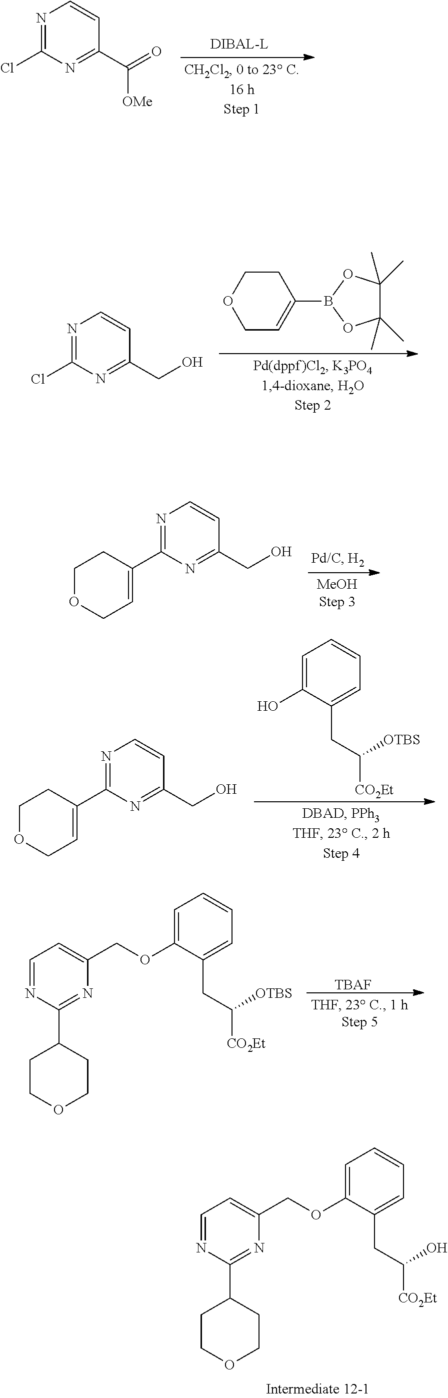

### Step 1

To a solution of compound methyl 2-chloropyrimidine-4-carboxylate (5.00 g, 28.9 mmol) in dichloromethane (100 mL) was added DIBAL-H (1 M, 57.9 mL, 2.00 eq) at 0° C. The mixture was stirred at 20° C. for 16 hours. The reaction was quenched with 10% citric acid (aqueous) and stirred at 20° C. for 30 minutes. The residue was partitioned between EtOAc (80 mL) and water (40 mL). The aqueous layer was extracted with EtOAc (40 mL, 2 times). The combined organic layers were dried over Na₂SO₄, filtered, and concentrated in vacuo to obtain a yellow solid (2.10 g, 14.5 mmol, 50.4% yield). The yellow solid was used in the next step without further purification.

LC/MS: m/z=145.0 [M+H]⁺ amu.

### Step 2

To a solution of (2-chloropyrimidin-4-yl)methanol (500 mg, 3.46 mmol), 2-(3,6-dihydro-2H-pyran-4-yl)-4,4,5,5-tetramethyl-1,3,2-dioxaborolane (799.2 mg, 3.80 mmol, 1.10 eq), K₃PO₄ (2.20 g, 10.38 mmol, 3.00 eq) and Pd(dppf)Cl₂ (253.1 mg, 345.9 umol, 0.10 eq) in dioxane (5 mL) was added H₂O (2 mL) degassed and purged with N₂ 3 times, and then the mixture was stirred at 80° C. for 3 hours under N₂ atmosphere. The reaction mixture was diluted with H₂O (30 mL) and extracted with EtOAc (30 mL, 3 times). The combined organic layers were washed with brine (30 mL), dried over Na₂SO₄, filtered and concentrated in vacuo to give a residue. The residue was purified by silica gel chromatography to yield (2-(3,6-dihydro-2H-pyran-4-yl)pyrimidin-4-yl)methanol (360 mg, 1.87 mmol, 54.15% yield) as a yellow solid.

LC/MS: m/z=193.1 [M+H]⁺ amu.

### Step 3

To a solution of (2-(3,6-dihydro-2H-pyran-4-yl)pyrimidin-4-yl)methanol (200 mg, 1.04 mmol, 1.00 eq) in MeOH (10 mL) was added Pd/C (0.1 g, 10% purity), and the reaction mixture was degassed and purged with H₂ (2.10 mg, 1.04 mmol, 1.00 eq) 3 times. The reaction was stirred at 20° C. for 1 hour under H₂ (15 psi) atmosphere. The reaction mixture was filtered and concentrated under reduced pressure to yield (2-(tetrahydro-2H-pyran-4-yl)pyrimidin-4-yl)methanol as a yellow solid (200 mg, 1.03 mmol, 98.96% yield).

### Step 4

To a mixture of (2-tetrahydropyran-4-ylpyrimidin-4-yl)methanol (143.66 mg, 0.7400 mmol), ethyl rac-(2S)-2-[tert-butyl(dimethyl)silyl]oxy-3-(2-hydroxyphenyl)propanoate (200. mg, 0.6200 mmol), and triphenylphosphine (0.24 g, 0.9200 mmol) in THE (6.1635 mL) was added DBAD (0.21 g, 0.9200 mmol). The resulting mixture was stirred at ambient temperature under inert atmosphere for 2 hours at which point the mixture was partitioned between EtOAc (50 mL) and water (20 mL). The aqueous layer was extracted with ethyl acetate (2×30 mL). The combined organics were dried over sodium sulfate and concentrated in vacuo. The crude product was purified by silica gel chromatography to give the desired product as a white solid.

LC/MS: m/z=501.3 [M+H]⁺ amu.

### Step 5

To a solution of ethyl rac-(2S)-2-[tert-butyl(dimethyl)silyl]oxy-3-[2-[(2-tetrahydropyran-4-ylpyrimidin-4-yl)methoxy]phenyl]propanoate (0.24 g, 0.4800 mmol) in THE (4.7933 mL) was added tertbutyl ammonium fluoride solution (1 M in THF, 0.72 mL, 0.72 mmol). The resulting mixture was stirred at ambient temperature. The reaction mixture was then purified by silica gel chromatography to give Intermediate 12-1 as an off-white solid.

LC/MS: m/z=387.2 [M+H]⁺ amu.

### Synthesis of Compound 111

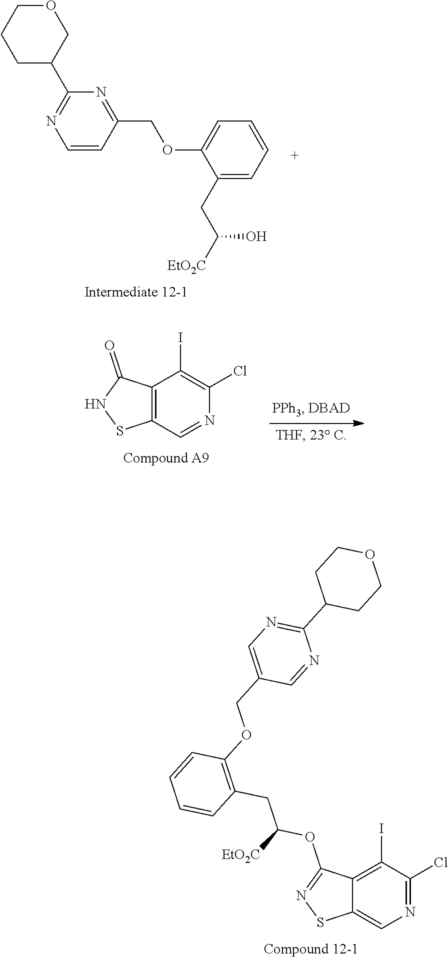 [SMILES: CCOC(=O)[C@@H](O)Cc1ccccc1OCc1ccnc(C2CCCOC2)n1 ; O=c1[nH]sc2cnc(Cl)c(I)c12 ; CCOC(=O)[C@@H](Cc1ccccc1OCc1cnc(C2CCOCC2)nc1)Oc1nsc2cnc(Cl)c(I)c12]

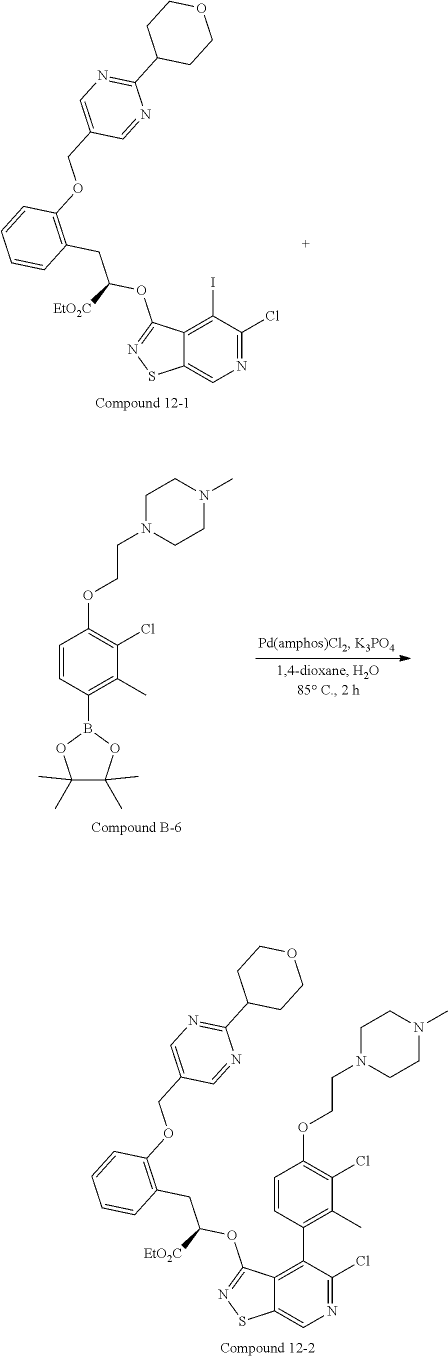

### Synthesis of Compound 111

Compound 12-1 was synthesized using compound A9, Intermediate 12-1, and following the general procedure used to synthesize Compound 3-2. Compound 12-2 was synthesized using Compound B-6, Compound 12-1, and following the general procedures used to synthesize Compound 3-3. Compound 111 was synthesized using Compound 12-2 and following the general procedures used to synthesize Compound 29. Compound 111 was obtained as an off-white solid.

¹H NMR (400 MHz, Methanol-d₄): δ 9.29 (s, 1H), 8.70 (d, J=5.2 Hz, 1H), 7.67 (d, J=5.2 Hz, 1H), 7.24-7.12 (m, 3H), 6.96 (d, J=8.3 Hz, 1H), 6.90 (td, J=7.4, 1.0 Hz, 1H), 6.53 (dd, J=7.4, 1.7 Hz, 1H), 5.57 (dd, J=10.0, 3.2 Hz, 1H), 5.29-5.05 (m, 2H), 4.40 (td, J=5.2, 1.8 Hz, 2H), 4.19-3.97 (m, 2H), 3.69-3.44 (m, 3H), 3.36 (d, J=3.3 Hz, 3H), 3.23-3.00 (m, 6H), 2.86 (s, 3H), 2.61-2.25 (m, 3H), 2.20-2.04 (m, 1H), 2.03-1.84 (m, 11H) (46 of 49 protons observed).

LC/MS: m/z=813.3 [M+H]⁺ amu.

### Synthesis of Compound 110

Compound 110 was synthesized following the general procedures used for Compound 111, wherein the corresponding vinyl boronate ester was used when performing the general procedures used to synthesize Intermediate 12-1. Compound 110 was obtained as an off-white solid.

¹H NMR (400 MHz, Methanol-d₄): δ 9.25 (s, 1H), 8.74-8.61 (m, 1H), 7.71-7.55 (m, 1H), 7.33-7.06 (m, 4H), 7.03-6.76 (m, 2H), 6.61-6.46 (m, 1H), 5.60-5.47 (m, 1H), 5.28-5.10 (m, 2H), 4.47-4.26 (m, 2H), 3.62-3.43 (m, 1H), 3.27-2.97 (m, 11H), 2.85 (s, 3H), 2.61-2.27 (m, 3H), 2.22-1.82 (m, 16H) (49 of 49 protons observed).

LC/MS: m/z=847.3 [M+H]⁺ amu.

Assignment of Absolute Chemical Configuration by Vibrational Circular Dichroism

### Experimental Protocol for Vibrational Circular Dichroism

A 50 mg/mL CDCl₃ solution of the chiral test compound is subjected to absolute configuration determination via vibrational circular dichroism (VCD) using a ChiralIR-2X spectrometer (BioTools, Inc) set to to 4 cm⁻¹ resolution and optimized at 1400 cm⁻¹. A sample of test compound in CDCl₃ is loaded into an SL-4 cell (International Crystal Laboratories) with BaF₂ windows and 100 m path length, and infrared (IR) and VCD spectra acquired in 24 one-hour blocks, which are averaged at the completion of the run. A 15-minute acquisition of neat (+)-α-pinene control is also acquired to yield a VCD spectrum in agreement with literature spectra. IR and VCD spectra were background-corrected using a 5-minute block acquisition of the empty instrument chamber. IR spectra are solvent corrected utilizing a one-hour block acquisition of CDCl₃. For enantiomeric pairs of compounds, final VCD spectra used for assignment are processed via enantiomer subtraction (half-difference).

### Computational Protocol

An arbitrarily chosen, but known, enantiomer of the compound in question is subjected to an exhaustive initial molecular mechanics-based conformational search (MMFF94 force field, 0.08 Å geometric RMSD cutoff, and 30 kcal/mol energy window) as implemented in MOE (Chemical Computing Group, Montreal, Calif.). The resultant conformers are checked to ensure the input chirality is retained. All MMFF94 conformers are then subjected to geometry optimization, harmonic frequency calculation, and VCD rotational strength evaluation with density functional theory. All final quantum mechanical calculations utilize the B3PW91 functional, cc-pVTZ basis (def2-TZVP basis for iodine-containing compounds) and the implicit IEFPCM chloroform solvation model as implemented in the Gaussian 16 program system (Rev. B.01; Frisch et al., Gaussian, Inc., Wallingford, Conn.). Resultant harmonic frequencies are scaled by 0.98. All structurally unique conformers are Boltzmann weighted by relative free energy at 298.15 K. The predicted IR and VCD frequencies and intensities are convolved using Lorentzian line shapes (γ=4 cm⁻¹) and summed using the respective Boltzmann weights to yield the final predicted IR and VCD spectra of the input enantiomer. The predicted VCD of the opposite corresponding enantiomer is easily generated by inversion of sign. From the generally excellent agreement between the predicted and measured IR and VCD spectra of the test article, the absolute configuration of the test article can generally be established in an unambiguous fashion.

Biological Experiments

### MCL1, BCL2 and BCLXL Affinity Assays

Recombinant MCL1, BCL2 and BCXL proteins were prepared in either an E. coli host derived from the BL21 strain or in HEK-293 cells. The recombinant proteins were subsequently tagged with DNA for qPCR detection. Streptavidin-coated magnetic beads were treated for 30 minutes at room temperature with the respective biotinylated peptide ligands for each recombinant protein to generate affinity resins for the assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining recombinant protein, liganded affinity beads, and test compounds in 1× binding buffer (20% SeaBlock, 0.17×PBS, 0.05% Tween 20, 6 mM DTT). Test compounds were prepared as 111X stocks in 100% DMSO. The compounds were then diluted directly into the assays such that the final concentration of DMSO was 0.9%. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The recombinant protein concentration in the eluates was measured by qPCR. K_{d}s were determined using an 11-point 3-fold compound dilution series with three DMSO control points. See Table 2. “A*” represents a K_{d} of 100 nM or less, “A” represents a K_{d} of 101 nM to 500 nM, “B” represents a K_{d} of 501 nM to 1,500 nM, and “C” represents a K_{d} of greater than 1,500 nM.

### Cell Line Growth Retardation Assay

Cells were seeded at densities of 1,000-5,000 cells per well in 48-well tissue culture plates. After a 24 h rest period, cells were treated with compound at 10 μM, 1 μM, 0.4 μM, 0.08 μM, 0.016 μM, and 0.0032 μM. A group of cells were treated with the vehicle in which the compound was prepared and served as a control. Prior to treatment, cells were counted and this count was used as a baseline for the calculation of growth inhibition. The cells were grown in the presence of compounds for 6 days and were counted on day 6. All cell counting was performed using a Synentec Cellavista plate imager. Growth inhibition was calculated as a ratio of cell population doublings in the presence of compound versus the absence of compound. If treatment resulted in a net loss of cells from baseline, percent lethality was defined as the decrease in cell numbers in treated wells compared with counts on day 1 of non-treated wells post-seeding. IC₅₀ values for each compound were calculated by fitting curves to data points from each dose-response assay using the Proc NLIN function in SAS for Windows version 9.2 (SAS Institute, Inc.).

### Designation of Sensitive and Resistant Cohorts and Calculation of Average IC₅₀ Values

Human cancer cell lines were grouped as “sensitive” or “resistant” to MCL1 inhibition based on whether their growth was retarded by AMG-176 (i.e., (1′S,11R,12S,14E,16S,16aR,18aR)-6′-Chloro-3′,4′,12,13,16,16a,17,18,18a,19-decahydro-16-methoxy-11,12-dimethyl-,Spiro[5,7-etheno-1H,11H-cyclobut[i][1,4]oxazepino[3,4-f][1,2,7]thiadiazacyclohexadecine-2(3H),1′(2′H)-naphthalen]-8(9H)-one 10,10-dioxide) or MIK665 (i.e., (R)-2-((5-(3-chloro-2-methyl-4-(2-(4-methylpiperazin-1-yl)ethoxy)phenyl)-6-(4-fluorophenyl)thieno[2,3-d]pyrimidin-4-yl)oxy)-3-(2-((2-(2-methoxyphenyl)pyrimidin-4-yl)methoxy)phenyl)propanoic acid) (data not shown; see Table 3). These sensitive and resistant cohorts were interrogated for response to each compound, and IC_{50S} were calculated for each cell line using the same technique described above. Average IC_{50S} for the sensitive (“AvgSen IC₅₀”) and resistant (“AvgRes IC₅₀”) cohorts were calculated as arithmetic means of the group, and fold differences (“Fold Diff”) between the resistant and sensitive cohorts were calculated by diving the average IC₅₀ for the resistant cohort by the average IC₅₀ for the sensitive cohort. See Table 2. “A” represents an IC₅₀ of 1 μM or less, “B” represents an IC₅₀ of greater than 1 μM to 5 μM, “C” represents an IC₅₀ of greater than 5 μM, “+” represents a fold difference of 10 or more, and “−” represents a fold difference less than 10.

### Caco-2 Assay (Pₐₚₚ A to B)

The degree of bi-directional human intestinal permeability for compounds was estimated using a Caco-2 cell permeability assay. Caco-2 cells were seeded onto polyethylene membranes in 96-well plates. The growth medium was refreshed every 4 to 5 days until cells formed a confluent cell monolayer. HBSS with 10 mM HEPES at pH 7.4 was used as the transport buffer. Compounds were tested at 2 μM bi-directionally in duplicate. Digoxin, nadolol and metoprolol were included as standards. Digoxin was tested at 10 μM bi-directionally in duplicate, while nadolol and metoprolol were tested at 2 μM in the A to B direction in duplicate. The final DMSO concentration was adjusted to less than 1% for all experiments. The plate was incubated for 2 hours in a CO₂ incubator at 37° C., with 5% CO₂ at saturated humidity. After incubation, all wells were mixed with acetonitrile containing an internal standard, and the plate was centrifuged at 4,000 rpm for 10 minutes. 100 μL supernatant was collected from each well and diluted with 100 μL distilled water for LC/MS/MS analysis. Concentrations of test and control compounds in starting solution, donor solution, and receiver solution were quantified by LC/MS/MS, using peak area ratio of analyte to internal standard.

The apparent permeability coefficient Pₐₚₚ (cm/s) was calculated using the equation:

Pₐₚₚ=(dCᵣ/dt)×Vᵣ/(A×C₀),

where dCᵣ/dt is the cumulative concentration of compound in the receiver chamber as a function of time (μM/s); Vᵣ is the solution volume in the receiver chamber (0.075 mL on the apical side, 0.25 mL on the basolateral side); A is the surface area for the transport, which is 0.0804 cm² for the area of the monolayer; and C₀ is the initial concentration in the donor chamber (μM).

The efflux ratio was calculated using the equation:

Efflux Ratio=Pₐₚₚ(BA)/Pₐₚₚ(AB)

Percent recovery was calculated using the equation:

% Recovery=100×[(Vᵣ×Cᵣ)+(V_{d}×C_{d})]/(V_{d}×C₀),

where Vd is the volume in the donor chambers, which are 0.075 mL on the apical side and 0.25 mL on the basolateral side; and Ca and Cᵣ are the final concentrations of transport compound in donor and receiver chambers, respectively.

### Measurement of Compound Metabolic Stability

The metabolic stability of compounds was determined in hepatocytes from human, mice and rats. Compounds were diluted to 5 μM in Williams' Medium E from 10 mM stock solutions. 10 μL of each compound was aliquoted into a well of a 96-well plate and reactions were started by aliquoting 40 μL of a 625,000 cells/mL suspension into each well. The plate was incubated at 37° C. with 5% CO₂. At each corresponding time point, the reaction was stopped by quenching with ACN containing internal standards (IS) at a 1:3. Plates were shaken at 500 rpm for 10 min, and then centrifuged at 3,220×g for 20 minutes. Supernatants were transferred to another 96-well plate containing a dilution solution. Supernatants were analyzed by LC/MS/MS.

The remaining percent of compound after incubation was calculated using the following equation:

$$\% Remaining Compund = \frac{Peak Area Ratios of Tested Compound vs . Internal Standard at End Point}{Peak Area Ratios of Tested Compound vs . Internal Standard at Start Point}$$

Compound half-life and CLᵢₙₜ were calculated using the following equations:

Cₜ=C₀*e^{-k*t} (first order kinetics); when Cₜ=½C₀, t½=1n2/k=0.693/k; and

CLᵢₙₜ=k/(1,000,000 cells/mL)

### Rodent Xenograft Models

Xenograft models of human cancer cell lines were established in six-week-old CD-1 athymic nude mice by subcutaneous injection of 1.0-3.0×10⁷ cells with or without 50% matrigel. When tumors reached an average size of 150-400 mm³, mice (n=8) were randomized into treatment groups. Tumor xenografts were measured with calipers three times per week, and tumor volume (in mm³) was determined by multiplying height×width×length. Statistical differences between treatment arms at specific time points were performed using a two-tailed paired Student t-test. Differences between groups were considered statistically significant at p<0.05. Compounds were formulated in 15% PS80 in dH₂O and dosed daily by intravenous injection (IV) for the first 5 days of the study. Data were analyzed using StudyLog software from StudyDirector (San Francisco, Calif.).

### Activity-Guided Selection of Inhibitors

Subgenera of MCL1 inhibitors having desirable properties were identified using a combination of in vitro data. In particular, the results from the assays described above (e.g., Cell Line Growth Retardation Assay, MCL1, BCL2 and BCLXL Affinity Assays, Caco-2 Assay (Pₐₚₚ A to B), Measurement of Compound Metabolic Stability, and Designation of Sensitivity and Resistant Cohorts and Calculation of Average IC₅₀ Values) were used to select compounds having structural and functional features defined in the subgenera of Formula (VIII).

In particular, a desirable property of compounds examined in sensitive and resistant cell lines, as described above, is having an average IC₅₀ for the drug-sensitive cell lines of Table 3 of about 1 μM or lower and having an average IC₅₀ for the drug-resistant cell lines of Table 3 of greater than 1 μM.

The skilled artisan would readily recognize that the results of additional in vitro assays (e.g., CYP enzymatic inhibition, hERG inhibition, compound solubility, target-specificity analysis), as well as the results of in vivo assays (e.g., rodent xenograft studies, rodent pharmacokinetic and single-dose saturation studies, rodent maximum tolerated dose studies, and oral bioavailability) could be used to identify other subgenera of MCL1 inhibitors, or to narrow subgenera determined using other results, for example, the subgenera of Formula (VIII).

TABLE 2

| | MCL1 | BCL2 | BCLXL | AvgSen | AvgRes | Fold |
|---|---|---|---|---|---|---|
| Cmpnd | K_{d} | K_{d} | K_{d} | IC₅₀ | IC₅₀ | Diff |
| 1 | C | C | C | C | C | − |
| 2 | B | C | C | B | C | − |
| 3 | C | C | C | C | C | − |
| 4 | B | C | C | C | C | − |
| 5 | C | C | C | C | C | − |
| 6 | A | C | C | B | C | − |
| 7 | C | C | C | C | C | − |
| 8 | C | | | | | |
| 9 | C | C | C | C | C | − |
| 10 | C | C | C | C | C | − |
| 11 | A | C | C | B | C | − |
| 12 | C | C | C | C | C | − |
| 13 | C | C | C | C | C | − |
| 14 | A | C | C | B | C | − |
| 15 | B | B | C | C | C | − |
| 16 | C | C | C | C | C | − |
| 17 | B | C | C | C | C | − |
| 18 | C | C | C | C | C | − |
| 19 | C | C | C | B | C | − |
| 20 | B | C | C | C | C | − |
| 21 | A | C | C | B | C | − |
| 22 | B | C | C | B | C | − |
| 23 | A | C | C | A | C | + |
| 24 | C | C | C | B | C | − |
| 25 | C | C | C | C | C | − |
| 26 | A | C | C | A | C | + |
| 27 | A | C | C | B | C | − |
| 28 | B | C | C | | | |
| 29 | A* | C | C | A | C | + |
| 30 | A* | C | C | B | C | − |
| 31 | C | C | C | B | C | − |
| 32 | A* | | | A | C | + |
| 33 | A* | | | A | C | + |
| 34 | A* | | | A | C | + |
| 35 | A* | | | A | C | + |
| 36 | A* | | | A | C | + |
| 37 | A | | | C | C | − |
| 38 | A* | | | A | C | + |
| 39 | B | | | B | B | − |
| 40 | A* | C | C | A | C | + |
| 41 | A* | C | C | B | C | − |
| 42 | A* | C | C | B | C | − |
| 43 | A* | C | C | B | C | − |
| 44 | A* | C | C | C | C | − |
| 45 | A* | C | C | B | C | − |
| 46 | A* | C | C | A | C | + |
| 47 | A* | C | C | A | C | + |
| 48 | A* | C | C | A | C | + |
| 49 | A* | C | C | B | C | − |
| 50 | A* | C | C | A | C | + |
| 51 | A* | C | C | B | C | − |
| 52 | B | C | C | C | C | − |
| 53 | A* | C | C | C | C | − |
| 54 | A* | C | C | B | C | − |
| 55 | A* | | | A | C | + |
| 56 | A* | | | A | C | + |
| 57 | A* | | | A | C | + |
| 58 | A* | | | A | C | + |
| 59 | A | | | B | C | − |
| 60 | C | | | B | C | − |
| 61 | A | | | A | C | + |
| 62 | B | | | B | C | − |
| 63 | B | | | B | C | − |
| 64 | A | | | C | C | − |
| 65 | A* | | | A | C | + |
| 66 | A* | | | A | C | + |
| 67 | A* | | | A | C | + |
| 68 | A* | | | B | C | − |
| 69 | A* | | | A | C | + |
| 70 | A | | | A | C | + |
| 71 | A | C | C | A | C | + |
| 72 | A* | C | C | A | C | + |
| 73 | A* | | | A | C | + |
| 74 | C | | | C | C | − |
| 75 | A | | | C | C | − |
| 76 | B | | | C | C | − |
| 77 | A* | | | B | C | − |
| 78 | A* | | | B | C | − |
| 79 | B | | | A | B | − |
| 80 | A* | | | A | C | + |
| 81 | A* | | | B | C | − |
| 82 | B | | | B | C | − |
| 83 | A* | | | A | C | + |
| 84 | A* | | | A | C | + |
| 85 | A* | | | A | C | + |
| 86 | B | | | B | C | − |
| 87 | A* | | | A | C | + |
| 88 | A* | | | A | C | + |
| 89 | B | | | B | C | − |
| 90 | A | | | B | C | − |
| 91 | C | | | C | C | − |
| 92 | B | C | C | B | C | − |
| 93 | A | | | B | C | − |
| 94 | C | | | C | C | − |
| 95 | C | | | C | C | − |
| 96 | C | | | C | C | − |
| 97 | B | | | B | C | − |
| 98 | C | | | A | B | − |
| 99 | C | | | C | C | − |
| 100 | B | | | B | C | − |
| 101 | C | | | C | C | − |
| 102 | A* | | | B | C | − |
| 103 | C | | | | | |
| 104 | A | | | B | C | − |
| 105 | | | | | | |
| 106 | A | | | B | C | − |
| 107 | A* | | | C | C | − |
| 108 | A* | | | B | C | − |
| 109 | A | | | B | C | − |
| 110 | A* | | | A | C | + |
| 111 | A* | | | A | C | + |
| 112 | A* | | | A | C | + |
| 113 | C | | | C | C | − |
| 114 | A | | | B | C | − |
| 115 | A | | | B | C | − |
| 116 | A* | | | A | C | + |
| 117 | A | | | B | C | − |
| 118 | A* | | | B | C | − |
| 119 | A* | | | A | C | + |
| 120 | | | | A | C | + |
| 121 | | | | A | C | + |
| 122 | | | | A | C | + |
| 123 | | | | A | C | + |
| 124 | | | | | | |
| 125 | | | | | | |
| 126 | | | | | | |

TABLE 3

| Cell Line Name | Cohort |
|---|---|
| NCI-H929 | Sensitive |
| SU-DHL-10 | Sensitive |
| Karpas 422 | Sensitive |
| MV4-11 | Sensitive |
| KMS-20 | Sensitive |
| AMO-1 | Sensitive |
| DoHH2 | Sensitive |
| MOLM-13 | Sensitive |
| ML-2 | Sensitive |
| OPM-2 | Sensitive |
| A427 | Sensitive |
| DB | Sensitive |
| NOMO-1 | Sensitive |
| NCI-H1568 | Sensitive |
| HCC1187 | Sensitive |
| SK-BR-3 | Sensitive |
| NCI-H1703 | Sensitive |
| MCF-7 | Sensitive |
| NCI-H23 | Sensitive |
| NCI-H2110 | Sensitive |
| HCC1954 | Sensitive |
| NCI-H661 | Sensitive |
| RC K8 | Resistant |
| F-36P | Resistant |
| U2932 | Resistant |
| ELF-153 | Resistant |
| JIMT-1 | Resistant |
| MDA-MB-231 | Resistant |
| NCI-H596 | Resistant |
| NCI-H647 | Resistant |

TABLE 4
E | RX3 | RX3a-1 | RX3a-2 | L3 | Yb | Z1 | RZ1

Imidazolyl | C1-C3 alkoxy | cyano | C1-C2 haloalkyl | CH2CH2 | Piperindinyl |
Imidazolyl | C1-C3 haloalkyl
Pyrimidinyl | C1-C4 alkyl | Halogen | amino | CH2CH2 | Morpholinyl | Pyrazolyl | C1-
C3 alkoxy
Pyrazolyl | C1-C4 alkyl | cyano | Halogen | CH2 | Piperazinyl | Benzyl | C1-C3 alkyl
Pyridinyl | C1-C4 alkyl | amino | H | CH2 | Morpholinyl | Pyrazolyl | C1-C3 haloalkyl
Pyrimidinyl | Pyridinyl | cyano | C1-C3 alkyl | CH2CH2 | N(CH3)2 | Imidazolyl | C1-
C3 alkoxy
Pyrimidinyl | C1-C4 alkyl | H | C1-C2 haloalkyl | CH2CH2 | N(CH3)2 | Cyclobutyl |
C1-C3 haloalkyl
Pyrimidinyl | C1-C3 alkoxy | amino | cyano | CH2 | Piperindinyl | Cyclobutyl | Halogen
Imidazolyl | C1-C3 alkoxy | cyano | amino | CH2 | Piperazinyl | Pyridinyl | C1-C3
haloalkyl
Pyrimidinyl | C1-C3 alkoxy | C1-C2 haloalkyl | cyano | CH2CH2 | N(CH3)2 |
Imidazolyl | C1-C3 haloalkyl
Pyrazolyl | C1-C3 alkoxy | H | C1-C2 haloalkyl | CH2CH2 | Piperazinyl | Pyridinyl |
Halogen
Pyrazolyl | Benzyl | cyano | C1-C3 alkyl | CH2 | Morpholinyl | Imidazolyl | C1-C3
alkoxy
Pyrazolyl | Pyridinyl | H | H | CH2 | Piperindinyl | Pyrazolyl | Halogen
Pyridinyl | C1-C4 alkyl | C1-C3 alkyl | amino | CH2 | Piperindinyl | Benzyl | Halogen
Imidazolyl | Benzyl | cyano | H | CH2 | Morpholinyl | Benzyl | C1-C3 alkyl
Pyrimidinyl | C1-C3 alkoxy | Halogen | Halogen | CH2CH2 | Piperindinyl | Imidazolyl |
C1-C3 haloalkyl
Pyrazolyl | C1-C3 alkoxy | C1-C3 alkyl | H | CH2CH2 | Piperindinyl | Pyridinyl | C1-
C3 haloalkyl
Pyridinyl | C1-C3 alkoxy | Halogen | Halogen | CH2CH2 | N(CH2CH3)2 | Pyrazolyl |
Halogen
Pyridinyl | Pyridinyl | cyano | cyano | CH2CH2 | Morpholinyl | Benzyl | C1-C3
haloalkyl
Pyridinyl | C1-C3 alkoxy | H | C1-C3 alkyl | CH2CH2 | Morpholinyl | Benzyl | C1-C3
alkyl
Imidazolyl | Benzyl | amino | H | CH2 | N(CH3)2 | Benzyl | C1-C3 alkoxy
Pyrazolyl | C1-C3 alkoxy | H | H | CH2 | N(CH2CH3)2 | Benzyl | C1-C3 haloalkyl
Pyrimidinyl | C1-C3 alkoxy | cyano | Halogen | CH2 | Piperazinyl | Cyclobutyl | C1-C3
alkoxy
Pyridinyl | Pyridinyl | C1-C3 alkyl | C1-C3 alkyl | CH2 | N(CH3)2 | Pyrazolyl | C1-C3
alkyl
Pyrazolyl | C1-C3 alkoxy | C1-C3 alkyl | cyano | CH2CH2 | Morpholinyl | Cyclobutyl |
Halogen
Pyridinyl | Pyridinyl | C1-C3 alkyl | Halogen | CH2 | Morpholinyl | Pyrazolyl | Halogen
Imidazolyl | C1-C3 alkoxy | C1-C3 alkyl | C1-C2 haloalkyl | CH2 | Morpholinyl |
Imidazolyl | C1-C3 alkyl
Pyridinyl | C1-C4 alkyl | amino | cyano | CH2 | Piperindinyl | Benzyl | C1-C3 alkoxy
Imidazolyl | Benzyl | C1-C3 alkyl | H | CH2CH2 | N(CH2CH3)2 | Imidazolyl | C1-C3
alkyl
Pyridinyl | Pyridinyl | amino | C1-C2 haloalkyl | CH2 | Piperindinyl | Cyclobutyl | C1-
C3 haloalkyl
Pyrimidinyl | C1-C4 alkyl | amino | Halogen | CH2CH2 | Piperindinyl | Pyridinyl |
Halogen
Pyrimidinyl | C1-C3 alkoxy | C1-C2 haloalkyl | Halogen | CH2 | N(CH2CH3)2 |
Pyrazolyl | C1-C3 alkoxy
Pyridinyl | C1-C4 alkyl | C1-C3 alkyl | H | CH2 | N(CH3)2 | Pyridinyl | C1-C3 alkoxy
Pyrimidinyl | Benzyl | C1-C3 alkyl | H | CH2CH2 | N(CH3)2 | Benzyl | C1-C3
haloalkyl
Pyrimidinyl | Pyridinyl | Halogen | amino | CH2CH2 | Piperazinyl | Benzyl | C1-C3
alkyl
Pyrazolyl | C1-C3 alkoxy | C1-C2 haloalkyl | H | CH2CH2 | N(CH2CH3)2 | Pyrazolyl |
C1-C3 alkoxy
Pyrazolyl | C1-C4 alkyl | C1-C2 haloalkyl | H | CH2CH2 | N(CH2CH3)2 | Imidazolyl |
C1-C3 haloalkyl
Pyridinyl | Pyridinyl | H | C1-C2 haloalkyl | CH2 | N(CH2CH3)2 | Cyclobutyl | Halogen
Imidazolyl | C1-C4 alkyl | Halogen | H | CH2 | Piperazinyl | Pyrazolyl | C1-C3 alkyl
Pyridinyl | C1-C4 alkyl | C1-C3 alkyl | C1-C3 alkyl | CH2 | N(CH3)2 | Pyridinyl | C1-
C3 alkyl
Pyrazolyl | C1-C3 alkoxy | amino | cyano | CH2 | N(CH2CH3)2 | Pyridinyl | C1-C3
alkoxy
Pyridinyl | C1-C3 alkoxy | C1-C2 haloalkyl | amino | CH2 | Piperazinyl | Pyrazolyl |
Halogen
Pyrimidinyl | Pyridinyl | cyano | H | CH2 | N(CH2CH3)2 | Imidazolyl | C1-C3 alkyl
Pyridinyl | C1-C3 alkoxy | amino | C1-C3 alkyl | CH2 | N(CH2CH3)2 | Imidazolyl | C1-
C3 haloalkyl
Pyrazolyl | Pyridinyl | C1-C2 haloalkyl | amino | CH2CH2 | Piperindinyl | Benzyl | C1-
C3 haloalkyl
Pyrazolyl | Pyridinyl | H | amino | CH2 | N(CH2CH3)2 | Pyrazolyl | C1-C3 haloalkyl
Imidazolyl | Benzyl | Halogen | amino | CH2 | Morpholinyl | Cyclobutyl | C1-C3 alkoxy
Imidazolyl | Pyridinyl | cyano | H | CH2CH2 | N(CH2CH3)2 | Cyclobutyl | C1-C3 alkyl
Imidazolyl | Pyridinyl | Halogen | cyano | CH2CH2 | Morpholinyl | Pyridinyl | C1-C3
haloalkyl
Pyrazolyl | Pyridinyl | cyano | Halogen | CH2CH2 | N(CH3)2 | Pyrazolyl | C1-C3
haloalkyl
Pyrazolyl | Benzyl | amino | H | CH2CH2 | Piperindinyl | Pyridinyl | C1-C3 alkyl
Pyrimidinyl | C1-C4 alkyl | C1-C2 haloalkyl | C1-C2 haloalkyl | CH2 | Piperindinyl |
Pyrazolyl | C1-C3 alkoxy
Pyridinyl | Benzyl | C1-C3 alkyl | amino | CH2 | Piperindinyl | Imidazolyl | C1-C3
haloalkyl
Imidazolyl | Pyridinyl | cyano | cyano | CH2CH2 | Piperindinyl | Pyridinyl | Halogen
Imidazolyl | Benzyl | cyano | C1-C3 alkyl | CH2CH2 | Morpholinyl | Cyclobutyl | C1-
C3 alkoxy
Pyrimidinyl | Pyridinyl | cyano | H | CH2CH2 | Piperazinyl | Imidazolyl | C1-C3
haloalkyl
Imidazolyl | Benzyl | amino | C1-C2 haloalkyl | CH2CH2 | N(CH2CH3)2 | Cyclobutyl |
C1-C3 alkyl
Pyrimidinyl | Pyridinyl | Halogen | C1-C2 haloalkyl | CH2 | Morpholinyl | Imidazolyl |
C1-C3 haloalkyl
Pyrimidinyl | C1-C4 alkyl | H | cyano | CH2CH2 | Piperazinyl | Pyrazolyl | C1-C3 alkyl
Pyrazolyl | C1-C4 alkyl | cyano | Halogen | CH2 | N(CH2CH3)2 | Imidazolyl | C1-C3
alkyl
Imidazolyl | Pyridinyl | cyano | C1-C3 alkyl | CH2CH2 | Morpholinyl | Cyclobutyl | C1-
C3 alkyl
Pyrimidinyl | Benzyl | H | C1-C2 haloalkyl | CH2CH2 | Piperazinyl | Benzyl | C1-C3
alkoxy
Imidazolyl | Benzyl | H | C1-C3 alkyl | CH2 | Piperindinyl | Pyrazolyl | C1-C3 haloalkyl
Pyrimidinyl | Benzyl | Halogen | C1-C3 alkyl | CH2 | Piperindinyl | Imidazolyl |
Halogen
Pyrimidinyl | C1-C4 alkyl | C1-C3 alkyl | amino | CH2CH2 | Piperazinyl | Pyridinyl |
Halogen
Pyrazolyl | C1-C4 alkyl | H | Halogen | CH2 | N(CH2CH3)2 | Cyclobutyl | C1-C3
haloalkyl
Pyrazolyl | C1-C4 alkyl | H | C1-C3 alkyl | CH2CH2 | Piperazinyl | Benzyl | C1-C3
alkoxy
Pyridinyl | Benzyl | amino | C1-C2 haloalkyl | CH2CH2 | Piperazinyl | Benzyl | C1-C3
haloalkyl
Pyrazolyl | Pyridinyl | H | cyano | CH2 | Piperindinyl | Cyclobutyl | C1-C3 alkyl
Pyrazolyl | Benzyl | amino | C1-C2 haloalkyl | CH2CH2 | Piperindinyl | Pyridinyl | C1-
C3 alkoxy
Pyrimidinyl | Benzyl | cyano | Halogen | CH2 | Morpholinyl | Imidazolyl | C1-C3
haloalkyl
Pyrimidinyl | C1-C4 alkyl | Halogen | H | CH2CH2 | Piperazinyl | Benzyl | C1-C3
haloalkyl
Pyrazolyl | Pyridinyl | C1-C2 haloalkyl | Halogen | CH2CH2 | N(CH3)2 | Pyridinyl |
C1-C3 alkoxy
Pyridinyl | Benzyl | Halogen | cyano | CH2CH2 | Piperazinyl | Cyclobutyl | C1-C3
alkoxy
Pyridinyl | C1-C4 alkyl | amino | amino | CH2 | Morpholinyl | Pyridinyl | C1-C3 alkoxy
Imidazolyl | Pyridinyl | C1-C3 alkyl | H | CH2 | Piperindinyl | Benzyl | C1-C3 haloalkyl
Pyrimidinyl | C1-C4 alkyl | amino | C1-C3 alkyl | CH2CH2 | N(CH2CH3)2 | Benzyl |
C1-C3 alkyl
Imidazolyl | Pyridinyl | H | cyano | CH2 | Piperazinyl | Cyclobutyl | Halogen
Pyrimidinyl | C1-C4 alkyl | Halogen | H | CH2 | N(CH3)2 | Pyrazolyl | Halogen
Pyrazolyl | Benzyl | amino | H | CH2 | N(CH3)2 | Benzyl | C1-C3 alkoxy
Pyrazolyl | C1-C4 alkyl | amino | cyano | CH2 | N(CH3)2 | Pyridinyl | C1-C3 haloalkyl
Pyrimidinyl | Benzyl | Halogen | H | CH2 | N(CH3)2 | Imidazolyl | C1-C3 alkoxy
Pyrazolyl | C1-C3 alkoxy | C1-C2 haloalkyl | H | CH2CH2 | Piperindinyl | Cyclobutyl |
C1-C3 alkoxy
Pyridinyl | Pyridinyl | Halogen | C1-C3 alkyl | CH2CH2 | Piperindinyl | Pyridinyl | C1-
C3 haloalkyl
Pyrimidinyl | Pyridinyl | Halogen | H | CH2 | N(CH2CH3)2 | Pyridinyl | Halogen
Imidazolyl | Pyridinyl | cyano | amino | CH2CH2 | Morpholinyl | Pyrazolyl | C1-C3
haloalkyl
Imidazolyl | C1-C3 alkoxy | C1-C3 alkyl | cyano | CH2CH2 | Morpholinyl | Benzyl |
C1-C3 alkoxy
Pyridinyl | Pyridinyl | cyano | Halogen | CH2CH2 | N(CH3)2 | Imidazolyl | C1-C3
haloalkyl
Pyrimidinyl | Benzyl | cyano | amino | CH2 | Piperazinyl | Benzyl | C1-C3 haloalkyl
Pyridinyl | Pyridinyl | amino | C1-C3 alkyl | CH2 | N(CH2CH3)2 | Benzyl | C1-C3
haloalkyl
Pyrazolyl | Benzyl | C1-C2 haloalkyl | H | CH2CH2 | Piperazinyl | Benzyl | C1-C3 alkyl
Imidazolyl | C1-C4 alkyl | cyano | cyano | CH2CH2 | N(CH2CH3)2 | Imidazolyl | C1-
C3 alkyl
Pyrazolyl | C1-C4 alkyl | Halogen | C1-C2 haloalkyl | CH2 | Morpholinyl | Cyclobutyl |
C1-C3 alkoxy
Pyrimidinyl | Pyridinyl | cyano | H | CH2 | Morpholinyl | Pyridinyl | Halogen
Imidazolyl | Benzyl | amino | amino | CH2 | Piperazinyl | Pyrazolyl | C1-C3 alkyl
Pyrazolyl | C1-C4 alkyl | H | amino | CH2CH2 | N(CH2CH3)2 | Cyclobutyl | C1-C3
alkyl
Pyrazolyl | Pyridinyl | amino | H | CH2 | Piperindinyl | Cyclobutyl | C1-C3 alkyl
Pyrimidinyl | Benzyl | H | H | CH2CH2 | Piperindinyl | Imidazolyl | Halogen
Imidazolyl | C1-C4 alkyl | H | H | CH2 | Piperindinyl | Imidazolyl | C1-C3 alkoxy
Pyrazolyl | Pyridinyl | C1-C3 alkyl | C1-C2 haloalkyl | CH2 | N(CH3)2 | Imidazolyl |
C1-C3 alkoxy
Pyrimidinyl | C1-C3 alkoxy | Halogen | amino | CH2CH2 | Piperazinyl | Cyclobutyl |
Halogen
Pyrimidinyl | C1-C4 alkyl | Halogen | amino | CH2 | Piperazinyl | Cyclobutyl | Halogen
Imidazolyl | Pyridinyl | Halogen | cyano | CH2CH2 | N(CH2CH3)2 | Benzyl | C1-C3
alkoxy
Imidazolyl | C1-C3 alkoxy | amino | H | CH2CH2 | Morpholinyl | Benzyl | C1-C3
alkoxy
Pyrimidinyl | Pyridinyl | cyano | C1-C3 alkyl | CH2CH2 | Piperazinyl | Imidazolyl |
Halogen
Pyrimidinyl | C1-C3 alkoxy | C1-C2 haloalkyl | cyano | CH2CH2 | Piperazinyl |
Cyclobutyl | C1-C3 alkyl
Imidazolyl | C1-C4 alkyl | H | C1-C2 haloalkyl | CH2CH2 | Piperazinyl | Pyridinyl |
Halogen
Pyrimidinyl | C1-C4 alkyl | cyano | cyano | CH2CH2 | N(CH3)2 | Pyridinyl | C1-C3
haloalkyl
Imidazolyl | C1-C3 alkoxy | Halogen | amino | CH2CH2 | Piperindinyl | Benzyl | C1-C3
alkoxy
Pyrimidinyl | C1-C4 alkyl | C1-C3 alkyl | cyano | CH2 | Morpholinyl | Pyrazolyl |
Halogen
Pyrazolyl | Pyridinyl | Halogen | Halogen | CH2CH2 | Morpholinyl | Cyclobutyl |
Halogen
Pyrazolyl | C1-C3 alkoxy | amino | cyano | CH2 | N(CH2CH3)2 | Pyrazolyl | C1-C3
haloalkyl
Pyrazolyl | C1-C3 alkoxy | H | amino | CH2CH2 | Morpholinyl | Pyridinyl | C1-C3
haloalkyl
Pyrazolyl | C1-C4 alkyl | Halogen | C1-C3 alkyl | CH2 | Piperazinyl | Benzyl | C1-C3
alkoxy
Imidazolyl | Pyridinyl | H | C1-C2 haloalkyl | CH2CH2 | N(CH3)2 | Pyrazolyl | C1-C3
alkoxy
Pyridinyl | Pyridinyl | C1-C2 haloalkyl | Halogen | CH2CH2 | Morpholinyl | Imidazolyl |
C1-C3 alkoxy
Pyrimidinyl | Benzyl | Halogen | amino | CH2CH2 | Morpholinyl | Pyridinyl | C1-C3
alkoxy
Pyrazolyl | C1-C4 alkyl | C1-C3 alkyl | C1-C2 haloalkyl | CH2CH2 | N(CH2CH3)2 |
Imidazolyl | C1-C3 alkyl
Pyrazolyl | C1-C4 alkyl | Halogen | Halogen | CH2CH2 | N(CH2CH3)2 | Benzyl |
Halogen
Pyrazolyl | C1-C4 alkyl | H | C1-C2 haloalkyl | CH2CH2 | Piperindinyl | Benzyl | C1-
C3 alkyl
Pyrazolyl | C1-C3 alkoxy | cyano | cyano | CH2CH2 | Piperazinyl | Imidazolyl | C1-C3
alkoxy
Pyridinyl | C1-C3 alkoxy | C1-C2 haloalkyl | C1-C3 alkyl | CH2 | N(CH2CH3)2 |
Cyclobutyl | C1-C3 alkyl
Pyrimidinyl | Benzyl | Halogen | amino | CH2 | Piperindinyl | Cyclobutyl | C1-C3
alkoxy
Pyrimidinyl | Pyridinyl | C1-C3 alkyl | C1-C2 haloalkyl | CH2 | N(CH2CH3)2 |
Imidazolyl | Halogen
Pyridinyl | Benzyl | amino | C1-C2 haloalkyl | CH2 | N(CH2CH3)2 | Imidazolyl | C1-
C3 alkyl
Pyrimidinyl | Pyridinyl | cyano | H | CH2 | Piperindinyl | Pyrazolyl | C1-C3 alkyl
Imidazolyl | Pyridinyl | C1-C2 haloalkyl | amino | CH2CH2 | Piperindinyl | Imidazolyl |
C1-C3 alkoxy
Pyrazolyl | Pyridinyl | C1-C2 haloalkyl | amino | CH2 | Piperindinyl | Pyrazolyl | C1-C3
haloalkyl
Pyrimidinyl | Benzyl | amino | amino | CH2 | N(CH3)2 | Cyclobutyl | C1-C3 alkoxy
Pyrimidinyl | C1-C3 alkoxy | C1-C3 alkyl | cyano | CH2CH2 | Piperindinyl | Cyclobutyl |
C1-C3 alkoxy
Imidazolyl | Benzyl | cyano | C1-C2 haloalkyl | CH2CH2 | Morpholinyl | Cyclobutyl |
C1-C3 haloalkyl
Imidazolyl | Pyridinyl | C1-C2 haloalkyl | C1-C3 alkyl | CH2CH2 | Piperazinyl |
Imidazolyl | Halogen
Pyrazolyl | Benzyl | C1-C2 haloalkyl | Halogen | CH2CH2 | Morpholinyl | Pyrazolyl |
C1-C3 alkyl
Pyridinyl | Pyridinyl | C1-C3 alkyl | cyano | CH2 | N(CH2CH3)2 | Imidazolyl | Halogen
Imidazolyl | Pyridinyl | Halogen | amino | CH2CH2 | N(CH2CH3)2 | Pyridinyl |
Halogen
Pyrimidinyl | Benzyl | H | C1-C3 alkyl | CH2 | Morpholinyl | Pyrazolyl | C1-C3
haloalkyl
Pyrazolyl | Pyridinyl | C1-C2 haloalkyl | C1-C3 alkyl | CH2CH2 | Piperazinyl |
Pyrazolyl | C1-C3 alkoxy
Pyridinyl | Pyridinyl | amino | C1-C3 alkyl | CH2 | Piperindinyl | Benzyl | C1-C3 alkoxy
Pyrimidinyl | Benzyl | amino | H | CH2CH2 | Piperindinyl | Cyclobutyl | C1-C3 alkoxy
Pyrazolyl | Pyridinyl | amino | amino | CH2CH2 | N(CH3)2 | Cyclobutyl | Halogen
Imidazolyl | C1-C3 alkoxy | C1-C2 haloalkyl | C1-C2 haloalkyl | CH2 | Morpholinyl |
Pyrazolyl | C1-C3 haloalkyl
Pyrazolyl | Benzyl | H | H | CH2 | Piperindinyl | Cyclobutyl | C1-C3 alkyl
Pyridinyl | C1-C4 alkyl | H | Halogen | CH2 | Morpholinyl | Benzyl | C1-C3 alkyl
Pyrimidinyl | Benzyl | amino | cyano | CH2CH2 | N(CH2CH3)2 | Pyridinyl | Halogen
Pyridinyl | Benzyl | cyano | C1-C3 alkyl | CH2 | Piperindinyl | Cyclobutyl | C1-C3
haloalkyl
Imidazolyl | C1-C3 alkoxy | amino | H | CH2 | N(CH2CH3)2 | Pyrazolyl | C1-C3
haloalkyl
Pyrazolyl | C1-C4 alkyl | C1-C3 alkyl | Halogen | CH2CH2 | Piperazinyl | Pyrazolyl |
C1-C3 alkyl
Imidazolyl | C1-C3 alkoxy | C1-C2 haloalkyl | cyano | CH2CH2 | Piperazinyl |
Pyridinyl | Halogen
Pyridinyl | C1-C4 alkyl | C1-C3 alkyl | amino | CH2CH2 | N(CH2CH3)2 | Pyridinyl |
C1-C3 alkoxy
Pyrimidinyl | C1-C3 alkoxy | Halogen | H | CH2CH2 | N(CH2CH3)2 | Pyridinyl | C1-
C3 haloalkyl
Pyridinyl | Benzyl | amino | C1-C2 haloalkyl | CH2 | Morpholinyl | Pyridinyl | C1-C3
alkyl
Pyridinyl | C1-C4 alkyl | amino | C1-C3 alkyl | CH2CH2 | N(CH2CH3)2 | Pyrazolyl |
C1-C3 alkoxy
Imidazolyl | C1-C4 alkyl | cyano | C1-C2 haloalkyl | CH2 | N(CH3)2 | Benzyl | C1-C3
alkoxy
Pyrazolyl | Pyridinyl | H | amino | CH2CH2 | Piperazinyl | Pyrazolyl | C1-C3 alkyl
Pyrazolyl | Benzyl | cyano | amino | CH2CH2 | N(CH2CH3)2 | Benzyl | C1-C3 alkoxy
Pyridinyl | Pyridinyl | cyano | C1-C2 haloalkyl | CH2CH2 | Piperazinyl | Pyrazolyl | C1-
C3 alkyl
Pyridinyl | C1-C3 alkoxy | C1-C2 haloalkyl | C1-C3 alkyl | CH2 | N(CH3)2 |
Cyclobutyl | C1-C3 haloalkyl
Imidazolyl | C1-C4 alkyl | C1-C2 haloalkyl | C1-C3 alkyl | CH2CH2 | N(CH3)2 |
Benzyl | C1-C3 alkyl
Imidazolyl | C1-C3 alkoxy | C1-C3 alkyl | C1-C2 haloalkyl | CH2CH2 | Piperindinyl |
Imidazolyl | C1-C3 alkyl
Pyrazolyl | Benzyl | amino | Halogen | CH2 | Morpholinyl | Pyridinyl | C1-C3 alkoxy
Pyrimidinyl | C1-C3 alkoxy | H | H | CH2 | N(CH3)2 | Pyrazolyl | C1-C3 alkyl
Pyridinyl | Pyridinyl | Halogen | Halogen | CH2 | Piperindinyl | Imidazolyl | C1-C3
alkoxy
Pyridinyl | C1-C4 alkyl | amino | cyano | CH2 | N(CH2CH3)2 | Benzyl | Halogen
Pyrazolyl | C1-C4 alkyl | C1-C2 haloalkyl | amino | CH2 | N(CH2CH3)2 | Benzyl |
Halogen
Pyrazolyl | C1-C4 alkyl | H | amino | CH2 | N(CH2CH3)2 | Imidazolyl | Halogen
Imidazolyl | C1-C3 alkoxy | Halogen | C1-C2 haloalkyl | CH2CH2 | Piperindinyl |
Cyclobutyl | C1-C3 alkyl
Pyrazolyl | Pyridinyl | amino | C1-C2 haloalkyl | CH2 | N(CH2CH3)2 | Cyclobutyl | C1-
Imidazolyl | C1-C4 alkyl | H | cyano | CH2 | Piperazinyl | Pyrazolyl | C1-C3 haloalkyl
Pyrazolyl | C1-C4 alkyl | Halogen | amino | CH2CH2 | N(CH3)2 | Pyridinyl | C1-C3
haloalkyl
Pyrazolyl | Benzyl | amino | H | CH2CH2 | N(CH2CH3)2 | Cyclobutyl | C1-C3 alkoxy
Pyrazolyl | Benzyl | H | Halogen | CH2CH2 | Piperazinyl | Cyclobutyl | C1-C3 alkoxy
Imidazolyl | C1-C3 alkoxy | cyano | amino | CH2 | N(CH2CH3)2 | Imidazolyl | C1-C3
haloalkyl
Pyrazolyl | C1-C3 alkoxy | amino | C1-C2 haloalkyl | CH2CH2 | Piperazinyl |
Imidazolyl | Halogen
Imidazolyl | Benzyl | amino | C1-C2 haloalkyl | CH2CH2 | N(CH2CH3)2 | Pyrazolyl |
C1-C3 alkoxy
Pyridinyl | Benzyl | H | C1-C3 alkyl | CH2CH2 | N(CH2CH3)2 | Pyridinyl | C1-C3
alkoxy
Pyrazolyl | C1-C3 alkoxy | amino | C1-C2 haloalkyl | CH2 | Piperazinyl | Pyrazolyl |
C1-C3 alkoxy
Pyrimidinyl | C1-C3 alkoxy | Halogen | Halogen | CH2 | Piperazinyl | Benzyl | C1-C3
alkoxy
Imidazolyl | C1-C4 alkyl | H | cyano | CH2CH2 | Morpholinyl | Cyclobutyl | C1-C3
haloalkyl
Pyrazolyl | C1-C4 alkyl | Halogen | H | CH2 | N(CH2CH3)2 | Imidazolyl | C1-C3 alkyl
Pyridinyl | C1-C3 alkoxy | C1-C2 haloalkyl | C1-C3 alkyl | CH2CH2 | Piperazinyl |
Imidazolyl | Halogen
Pyrazolyl | C1-C4 alkyl | amino | cyano | CH2 | Piperindinyl | Imidazolyl | Halogen
Pyrazolyl | Pyridinyl | C1-C2 haloalkyl | Halogen | CH2 | N(CH2CH3)2 | Pyrazolyl |
C1-C3 alkyl
Pyrazolyl | Pyridinyl | amino | C1-C2 haloalkyl | CH2CH2 | Piperazinyl | Pyrazolyl |
C1-C3 alkyl
Imidazolyl | C1-C4 alkyl | amino | cyano | CH2 | Morpholinyl | Cyclobutyl | C1-C3
alkoxy
Imidazolyl | Pyridinyl | C1-C2 haloalkyl | Halogen | CH2CH2 | N(CH2CH3)2 |
Cyclobutyl | Halogen
Pyrimidinyl | Benzyl | C1-C3 alkyl | amino | CH2CH2 | Piperindinyl | Pyrazolyl | C1-
C3 alkoxy
Pyridinyl | C1-C4 alkyl | Halogen | Halogen | CH2 | Piperazinyl | Benzyl | Halogen
Imidazolyl | Benzyl | amino | C1-C3 alkyl | CH2 | Piperazinyl | Pyridinyl | Halogen
Pyrimidinyl | C1-C4 alkyl | H | H | CH2 | Morpholinyl | Imidazolyl | C1-C3 alkoxy
Imidazolyl | Benzyl | cyano | cyano | CH2CH2 | Piperazinyl | Pyrazolyl | C1-C3 alkoxy
Pyridinyl | Benzyl | C1-C2 haloalkyl | Halogen | CH2 | Piperindinyl | Pyridinyl | C1-C3
alkyl
Pyridinyl | Pyridinyl | amino | Halogen | CH2CH2 | Morpholinyl | Pyrazolyl | C1-C3
alkoxy
Pyrimidinyl | C1-C3 alkoxy | C1-C3 alkyl | cyano | CH2CH2 | Piperazinyl | Pyrazolyl |
Halogen
Pyrazolyl | Benzyl | C1-C3 alkyl | C1-C3 alkyl | CH2CH2 | N(CH2CH3)2 | Imidazolyl |
C1-C3 alkoxy
Pyrazolyl | Benzyl | C1-C2 haloalkyl | C1-C2 haloalkyl | CH2 | Piperindinyl | Benzyl |
C1-C3 alkoxy
Pyridinyl | Benzyl | C1-C2 haloalkyl | C1-C2 haloalkyl | CH2 | N(CH2CH3)2 |
Cyclobutyl | C1-C3 haloalkyl
Pyrimidinyl | C1-C3 alkoxy | C1-C3 alkyl | amino | CH2CH2 | Morpholinyl | Benzyl |
C1-C3 alkyl
Imidazolyl | C1-C3 alkoxy | amino | amino | CH2CH2 | Piperindinyl | Benzyl | C1-C3
haloalkyl
Pyrimidinyl | Pyridinyl | C1-C3 alkyl | Halogen | CH2 | Piperazinyl | Cyclobutyl | C1-
C3 haloalkyl
Pyrazolyl | C1-C4 alkyl | H | amino | CH2 | Morpholinyl | Benzyl | C1-C3 alkoxy
Pyrimidinyl | Pyridinyl | C1-C2 haloalkyl | amino | CH2 | Morpholinyl | Benzyl |
Halogen
Pyrimidinyl | C1-C4 alkyl | cyano | H | CH2CH2 | N(CH3)2 | Imidazolyl | C1-C3 alkyl
Pyridinyl | Pyridinyl | H | C1-C2 haloalkyl | CH2CH2 | Piperazinyl | Imidazolyl | C1-C3
haloalkyl
Pyrimidinyl | C1-C3 alkoxy | C1-C3 alkyl | C1-C2 haloalkyl | CH2 | N(CH2CH3)2 |
Imidazolyl | C1-C3 alkoxy
Pyrazolyl | Benzyl | H | C1-C2 haloalkyl | CH2CH2 | N(CH2CH3)2 | Cyclobutyl | C1-
C3 alkyl
Pyridinyl | Pyridinyl | Halogen | amino | CH2CH2 | N(CH2CH3)2 | Pyrazolyl | C1-C3
alkoxy
Pyridinyl | C1-C4 alkyl | C1-C2 haloalkyl | H | CH2 | Piperindinyl | Imidazolyl | C1-C3
alkoxy
Pyrimidinyl | C1-C3 alkoxy | C1-C3 alkyl | H | CH2 | Piperazinyl | Benzyl | C1-C3
alkoxy
Pyrimidinyl | C1-C3 alkoxy | cyano | Halogen | CH2 | N(CH2CH3)2 | Pyridinyl | C1-C3
haloalkyl
Imidazolyl | Pyridinyl | cyano | H | CH2CH2 | Morpholinyl | Imidazolyl | C1-C3
haloalkyl
Pyrazolyl | C1-C3 alkoxy | C1-C3 alkyl | Halogen | CH2CH2 | N(CH3)2 | Imidazolyl |
C1-C3 alkoxy
Pyridinyl | Pyridinyl | cyano | Halogen | CH2 | Morpholinyl | Benzyl | C1-C3 alkyl
Pyrimidinyl | C1-C3 alkoxy | cyano | H | CH2 | Piperazinyl | Benzyl | C1-C3 alkoxy
Pyridinyl | Pyridinyl | C1-C2 haloalkyl | cyano | CH2CH2 | N(CH3)2 | Cyclobutyl | C1-
C3 haloalkyl
Imidazolyl | Pyridinyl | C1-C3 alkyl | amino | CH2 | N(CH3)2 | Pyrazolyl | C1-C3 alkyl
Pyridinyl | Benzyl | amino | cyano | CH2CH2 | Piperazinyl | Cyclobutyl | C1-C3 alkoxy
Pyridinyl | C1-C4 alkyl | cyano | Halogen | CH2CH2 | Piperindinyl | Benzyl | C1-C3
alkoxy
Pyrazolyl | C1-C4 alkyl | amino | H | CH2CH2 | Morpholinyl | Pyridinyl | C1-C3
haloalkyl
Pyridinyl | C1-C4 alkyl | H | cyano | CH2 | N(CH2CH3)2 | Imidazolyl | Halogen
Pyrimidinyl | C1-C3 alkoxy | C1-C2 haloalkyl | amino | CH2CH2 | Piperazinyl |
Imidazolyl | C1-C3 alkyl
Imidazolyl | Pyridinyl | cyano | C1-C3 alkyl | CH2 | N(CH3)2 | Pyrazolyl | C1-C3 alkyl
Imidazolyl | Pyridinyl | H | cyano | CH2 | Morpholinyl | Imidazolyl | Halogen
Pyrimidinyl | Pyridinyl | amino | Halogen | CH2CH2 | N(CH2CH3)2 | Benzyl | C1-C3
alkoxy
Pyrimidinyl | Benzyl | cyano | C1-C3 alkyl | CH2 | Piperindinyl | Benzyl | C1-C3
haloalkyl
Pyrazolyl | Pyridinyl | Halogen | amino | CH2 | N(CH2CH3)2 | Cyclobutyl | Halogen
Pyridinyl | Pyridinyl | cyano | C1-C2 haloalkyl | CH2 | N(CH2CH3)2 | Pyrazolyl | C1-
C3 haloalkyl
Pyridinyl | C1-C3 alkoxy | Halogen | C1-C3 alkyl | CH2 | Piperindinyl | Pyrazolyl | C1-
C3 alkoxy
Imidazolyl | Benzyl | amino | C1-C2 haloalkyl | CH2 | Piperindinyl | Pyridinyl | C1-C3
haloalkyl
Pyrazolyl | Benzyl | cyano | C1-C2 haloalkyl | CH2CH2 | Piperazinyl | Benzyl |
Halogen
Pyridinyl | Pyridinyl | C1-C3 alkyl | C1-C2 haloalkyl | CH2CH2 | N(CH2CH3)2 |
Benzyl | C1-C3 alkyl
Pyrimidinyl | Benzyl | C1-C3 alkyl | amino | CH2CH2 | Piperazinyl | Cyclobutyl |
Halogen
Pyrazolyl | Benzyl | Halogen | cyano | CH2 | N(CH2CH3)2 | Pyrazolyl | Halogen
Pyrazolyl | Pyridinyl | cyano | H | CH2 | Piperindinyl | Imidazolyl | C1-C3 haloalkyl
Imidazolyl | Pyridinyl | H | H | CH2CH2 | N(CH2CH3)2 | Cyclobutyl | Halogen
Pyrimidinyl | Benzyl | H | cyano | CH2 | Piperindinyl | Pyridinyl | Halogen
Pyrazolyl | C1-C3 alkoxy | H | cyano | CH2 | Piperazinyl | Pyridinyl | C1-C3 haloalkyl
Imidazolyl | C1-C3 alkoxy | C1-C2 haloalkyl | H | CH2 | N(CH2CH3)2 | Pyrazolyl |
C1-C3 alkyl
Pyrimidinyl | Benzyl | C1-C2 haloalkyl | C1-C3 alkyl | CH2 | Piperazinyl | Cyclobutyl |
C1-C3 alkoxy
Imidazolyl | Benzyl | cyano | Halogen | CH2 | N(CH3)2 | Imidazolyl | C1-C3 haloalkyl
Imidazolyl | Pyridinyl | C1-C3 alkyl | C1-C2 haloalkyl | CH2CH2 | Piperazinyl |
Cyclobutyl | C1-C3 haloalkyl
Pyrimidinyl | Pyridinyl | Halogen | H | CH2CH2 | N(CH3)2 | Pyrazolyl | Halogen
Pyrazolyl | C1-C4 alkyl | cyano | C1-C3 alkyl | CH2CH2 | N(CH3)2 | Imidazolyl |
Halogen
Pyrimidinyl | Benzyl | cyano | cyano | CH2 | N(CH3)2 | Cyclobutyl | C1-C3 alkoxy
Pyrimidinyl | Pyridinyl | cyano | cyano | CH2 | N(CH3)2 | Imidazolyl | Halogen
Pyrazolyl | Benzyl | H | C1-C3 alkyl | CH2 | N(CH2CH3)2 | Cyclobutyl | C1-C3 alkyl
Pyrazolyl | C1-C3 alkoxy | H | C1-C2 haloalkyl | CH2CH2 | N(CH2CH3)2 | Cyclobutyl |
Halogen
Imidazolyl | Pyridinyl | cyano | amino | CH2 | Piperindinyl | Imidazolyl | C1-C3 alkyl
Pyrimidinyl | Pyridinyl | amino | H | CH2 | Morpholinyl | Pyrazolyl | C1-C3 alkyl
Pyridinyl | Benzyl | C1-C3 alkyl | Halogen | CH2CH2 | Piperindinyl | Cyclobutyl | C1-
C3 alkoxy
Pyrazolyl | Pyridinyl | C1-C3 alkyl | amino | CH2 | N(CH3)2 | Pyrazolyl | C1-C3
haloalkyl
Pyridinyl | Benzyl | Halogen | Halogen | CH2CH2 | N(CH3)2 | Pyrazolyl | C1-C3 alkyl
Pyrimidinyl | Pyridinyl | C1-C3 alkyl | C1-C2 haloalkyl | CH2CH2 | N(CH2CH3)2 |
Pyridinyl | C1-C3 alkoxy
Pyrimidinyl | C1-C3 alkoxy | Halogen | cyano | CH2 | Piperindinyl | Cyclobutyl | C1-C3
alkoxy
Pyrimidinyl | Benzyl | amino | Halogen | CH2 | Morpholinyl | Imidazolyl | C1-C3
alkoxy
Pyrazolyl | C1-C4 alkyl | H | C1-C3 alkyl | CH2CH2 | N(CH2CH3)2 | Cyclobutyl | C1-
C3 alkoxy
Pyridinyl | Pyridinyl | cyano | H | CH2CH2 | Piperindinyl | Benzyl | Halogen
Pyrimidinyl | Benzyl | cyano | Halogen | CH2CH2 | Piperindinyl | Pyridinyl | Halogen
Pyridinyl | Benzyl | C1-C2 haloalkyl | cyano | CH2CH2 | Piperindinyl | Pyrazolyl |
Halogen
Pyridinyl | C1-C4 alkyl | C1-C3 alkyl | Halogen | CH2 | Piperindinyl | Cyclobutyl | C1-
C3 haloalkyl
Pyrimidinyl | C1-C3 alkoxy | Halogen | cyano | CH2 | Morpholinyl | Imidazolyl | C1-C3
alkyl
Pyridinyl | C1-C3 alkoxy | H | C1-C3 alkyl | CH2CH2 | N(CH2CH3)2 | Imidazolyl |
Halogen
Imidazolyl | Pyridinyl | H | Halogen | CH2 | Morpholinyl | Imidazolyl | Halogen
Imidazolyl | C1-C4 alkyl | C1-C3 alkyl | amino | CH2CH2 | Piperazinyl | Pyrazolyl |
C1-C3 haloalkyl
Pyridinyl | C1-C3 alkoxy | C1-C3 alkyl | amino | CH2 | N(CH3)2 | Imidazolyl | Halogen
Pyrazolyl | C1-C4 alkyl | C1-C2 haloalkyl | Halogen | CH2CH2 | N(CH3)2 | Pyridinyl |
C1-C3 haloalkyl
Pyridinyl | Benzyl | H | C1-C3 alkyl | CH2 | Piperazinyl | Benzyl | Halogen
Pyridinyl | Pyridinyl | C1-C3 alkyl | C1-C2 haloalkyl | CH2 | Piperazinyl | Benzyl | C1-
C3 alkoxy
Pyrazolyl | C1-C4 alkyl | C1-C2 haloalkyl | amino | CH2CH2 | Morpholinyl | Pyrazolyl |
C1-C3 alkyl
Pyrimidinyl | Pyridinyl | C1-C2 haloalkyl | H | CH2 | Piperazinyl | Imidazolyl | C1-C3
alkyl
Pyrazolyl | C1-C3 alkoxy | Halogen | C1-C2 haloalkyl | CH2 | Piperindinyl | Cyclobutyl |
C1-C3 alkyl
Pyrimidinyl | Pyridinyl | cyano | cyano | CH2 | N(CH2CH3)2 | Imidazolyl | C1-C3 alkyl
Pyrazolyl | Pyridinyl | C1-C2 haloalkyl | amino | CH2CH2 | Piperazinyl | Benzyl |
Halogen
Pyrimidinyl | Benzyl | amino | C1-C2 haloalkyl | CH2CH2 | Piperindinyl | Pyrazolyl |
C1-C3 haloalkyl
Pyrimidinyl | Benzyl | H | H | CH2 | Piperazinyl | Benzyl | Halogen
Pyrazolyl | C1-C4 alkyl | Halogen | amino | CH2 | Morpholinyl | Benzyl | C1-C3 alkoxy
Pyrazolyl | C1-C3 alkoxy | amino | C1-C3 alkyl | CH2 | N(CH3)2 | Cyclobutyl | C1-C3
alkoxy
Imidazolyl | C1-C4 alkyl | H | Halogen | CH2 | Morpholinyl | Pyrazolyl | Halogen
Pyridinyl | C1-C3 alkoxy | C1-C2 haloalkyl | H | CH2 | Piperazinyl | Pyridinyl | C1-C3
haloalkyl
Pyridinyl | C1-C4 alkyl | amino | amino | CH2CH2 | N(CH2CH3)2 | Pyrazolyl | C1-C3
haloalkyl
Imidazolyl | C1-C3 alkoxy | C1-C2 haloalkyl | Halogen | CH2 | Piperazinyl | Cyclobutyl |
C1-C3 alkyl
Pyridinyl | C1-C3 alkoxy | C1-C2 haloalkyl | amino | CH2 | N(CH3)2 | Cyclobutyl | C1-
C3 haloalkyl
Pyridinyl | C1-C4 alkyl | C1-C3 alkyl | cyano | CH2 | Morpholinyl | Pyridinyl | C1-C3
alkyl
Pyrimidinyl | Benzyl | Halogen | C1-C2 haloalkyl | CH2 | Morpholinyl | Imidazolyl |
Halogen
Pyridinyl | Benzyl | C1-C3 alkyl | amino | CH2CH2 | Piperindinyl | Pyridinyl | Halogen
Imidazolyl | C1-C4 alkyl | H | C1-C3 alkyl | CH2CH2 | Morpholinyl | Pyrazolyl | C1-
C3 alkoxy
Pyrazolyl | C1-C4 alkyl | C1-C2 haloalkyl | amino | CH2CH2 | N(CH2CH3)2 | Benzyl |
Halogen
Pyrazolyl | C1-C3 alkoxy | amino | C1-C2 haloalkyl | CH2 | N(CH3)2 | Benzyl | C1-C3
alkyl
Imidazolyl | Benzyl | H | Halogen | CH2CH2 | N(CH2CH3)2 | Benzyl | C1-C3 haloalkyl
Imidazolyl | C1-C3 alkoxy | C1-C3 alkyl | C1-C3 alkyl | CH2 | N(CH2CH3)2 |
Cyclobutyl | C1-C3 alkoxy
Pyridinyl | Benzyl | C1-C3 alkyl | H | CH2CH2 | N(CH2CH3)2 | Pyridinyl | C1-C3
alkyl
Pyrazolyl | Benzyl | Halogen | Halogen | CH2 | N(CH2CH3)2 | Cyclobutyl | C1-C3
haloalkyl
Pyridinyl | Pyridinyl | amino | cyano | CH2 | Morpholinyl | Cyclobutyl | C1-C3 alkoxy
Imidazolyl | Pyridinyl | C1-C3 alkyl | Halogen | CH2 | Piperindinyl | Pyrazolyl | C1-C3
alkoxy
Pyridinyl | Pyridinyl | amino | Halogen | CH2 | N(CH3)2 | Pyrazolyl | C1-C3 alkyl
Pyrimidinyl | Pyridinyl | Halogen | amino | CH2 | Piperindinyl | Pyridinyl | Halogen
Pyridinyl | C1-C3 alkoxy | C1-C2 haloalkyl | amino | CH2CH2 | Piperazinyl |
Imidazolyl | C1-C3 alkoxy
Pyrazolyl | Pyridinyl | C1-C3 alkyl | cyano | CH2 | N(CH3)2 | Benzyl | C1-C3 haloalkyl
Pyridinyl | C1-C3 alkoxy | amino | H | CH2CH2 | N(CH3)2 | Benzyl | Halogen
Pyrazolyl | C1-C3 alkoxy | amino | cyano | CH2CH2 | Piperazinyl | Pyrazolyl | Halogen
Pyrimidinyl | C1-C3 alkoxy | Halogen | C1-C3 alkyl | CH2CH2 | Piperindinyl |
Cyclobutyl | C1-C3 alkyl
Pyrimidinyl | C1-C4 alkyl | H | amino | CH2 | Morpholinyl | Cyclobutyl | Halogen
Pyridinyl | C1-C4 alkyl | C1-C3 alkyl | H | CH2CH2 | Morpholinyl | Pyrazolyl |
Halogen
Pyridinyl | C1-C3 alkoxy | cyano | H | CH2CH2 | Morpholinyl | Pyridinyl | C1-C3
haloalkyl
Pyrimidinyl | C1-C3 alkoxy | C1-C2 haloalkyl | C1-C3 alkyl | CH2 | Piperazinyl |
Pyridinyl | C1-C3 alkoxy
Pyridinyl | Pyridinyl | C1-C2 haloalkyl | C1-C3 alkyl | CH2 | Piperazinyl | Pyrazolyl |
C1-C3 alkyl
Pyridinyl | Pyridinyl | Halogen | C1-C2 haloalkyl | CH2 | Morpholinyl | Imidazolyl |
Halogen
Imidazolyl | C1-C3 alkoxy | C1-C3 alkyl | H | CH2 | Morpholinyl | Imidazolyl | C1-C3
alkoxy
Pyrimidinyl | C1-C3 alkoxy | H | cyano | CH2CH2 | Piperazinyl | Benzyl | Halogen
Pyrimidinyl | Pyridinyl | Halogen | cyano | CH2 | N(CH2CH3)2 | Pyrazolyl | C1-C3
haloalkyl
Imidazolyl | C1-C3 alkoxy | H | C1-C2 haloalkyl | CH2 | Morpholinyl | Imidazolyl |
Halogen
Imidazolyl | C1-C4 alkyl | cyano | C1-C3 alkyl | CH2CH2 | Piperindinyl | Benzyl |
Halogen
Pyrazolyl | Benzyl | amino | C1-C3 alkyl | CH2 | Piperindinyl | Pyridinyl | Halogen
Pyrazolyl | Benzyl | H | C1-C3 alkyl | CH2 | Morpholinyl | Pyrazolyl | C1-C3 alkyl
Pyrazolyl | Pyridinyl | cyano | amino | CH2 | Piperazinyl | Imidazolyl | C1-C3 alkyl
Pyrimidinyl | Pyridinyl | C1-C3 alkyl | cyano | CH2CH2 | Piperazinyl | Imidazolyl | C1-
C3 alkoxy
Pyrimidinyl | C1-C4 alkyl | C1-C3 alkyl | H | CH2 | N(CH2CH3)2 | Pyridinyl | C1-C3
alkyl
Pyrazolyl | Pyridinyl | C1-C2 haloalkyl | H | CH2CH2 | N(CH2CH3)2 | Cyclobutyl |
Halogen
Pyridinyl | C1-C3 alkoxy | C1-C2 haloalkyl | H | CH2CH2 | N(CH2CH3)2 | Benzyl |
C1-C3 alkyl
Pyridinyl | C1-C4 alkyl | C1-C3 alkyl | C1-C3 alkyl | CH2CH2 | N(CH3)2 | Cyclobutyl |
C1-C3 haloalkyl
Pyridinyl | Benzyl | C1-C3 alkyl | H | CH2 | Morpholinyl | Pyridinyl | Halogen
Pyridinyl | Benzyl | Halogen | amino | CH2CH2 | Morpholinyl | Cyclobutyl | C1-C3
alkoxy
Pyrimidinyl | Benzyl | cyano | Halogen | CH2 | Piperazinyl | Pyridinyl | C1-C3 alkoxy
Pyrimidinyl | C1-C3 alkoxy | H | C1-C2 haloalkyl | CH2CH2 | Morpholinyl | Pyrazolyl |
C1-C3 alkyl
Pyrimidinyl | Pyridinyl | H | cyano | CH2 | Piperindinyl | Benzyl | C1-C3 alkoxy
Pyrazolyl | C1-C4 alkyl | C1-C2 haloalkyl | cyano | CH2CH2 | Piperindinyl | Pyridinyl |
C1-C3 alkoxy
Pyrazolyl | C1-C4 alkyl | Halogen | cyano | CH2 | N(CH2CH3)2 | Pyrazolyl | C1-C3
alkyl
Pyrimidinyl | Benzyl | C1-C2 haloalkyl | Halogen | CH2CH2 | N(CH2CH3)2 |
Cyclobutyl | C1-C3 alkoxy
Pyrazolyl | C1-C3 alkoxy | C1-C3 alkyl | Halogen | CH2CH2 | Piperindinyl | Pyridinyl |
C1-C3 haloalkyl
Pyrimidinyl | C1-C3 alkoxy | amino | H | CH2 | Piperindinyl | Pyridinyl | C1-C3 alkoxy
Pyrazolyl | C1-C3 alkoxy | cyano | C1-C3 alkyl | CH2CH2 | Piperindinyl | Pyrazolyl |
C1-C3 alkyl
Imidazolyl | Pyridinyl | amino | C1-C3 alkyl | CH2CH2 | N(CH2CH3)2 | Imidazolyl |
C1-C3 alkoxy
Imidazolyl | Pyridinyl | Halogen | H | CH2 | N(CH2CH3)2 | Pyridinyl | C1-C3 alkyl
Pyrimidinyl | C1-C4 alkyl | C1-C3 alkyl | amino | CH2CH2 | Piperazinyl | Pyrazolyl |
C1-C3 haloalkyl
Imidazolyl | C1-C4 alkyl | C1-C3 alkyl | C1-C3 alkyl | CH2 | Morpholinyl | Imidazolyl |
Halogen
Pyrazolyl | C1-C3 alkoxy | cyano | amino | CH2 | N(CH3)2 | Benzyl | Halogen
Pyridinyl | C1-C4 alkyl | cyano | Halogen | CH2 | N(CH2CH3)2 | Imidazolyl | C1-C3
haloalkyl
Pyrimidinyl | Pyridinyl | H | cyano | CH2 | Piperindinyl | Pyrazolyl | C1-C3 alkoxy
Pyrazolyl | C1-C3 alkoxy | amino | C1-C2 haloalkyl | CH2 | Piperindinyl | Imidazolyl |
C1-C3 alkyl
Pyrimidinyl | Pyridinyl | cyano | cyano | CH2 | N(CH2CH3)2 | Pyridinyl | C1-C3 alkoxy
Pyrimidinyl | C1-C4 alkyl | H | Halogen | CH2CH2 | Piperindinyl | Pyridinyl | C1-C3
alkyl
Pyrazolyl | C1-C4 alkyl | amino | amino | CH2 | Piperindinyl | Benzyl | C1-C3 alkyl
Pyridinyl | C1-C3 alkoxy | Halogen | H | CH2 | Piperindinyl | Pyrazolyl | C1-C3 alkoxy
Pyrimidinyl | C1-C3 alkoxy | C1-C2 haloalkyl | C1-C3 alkyl | CH2 | Piperazinyl |
Pyrazolyl | C1-C3 alkoxy
Pyrazolyl | C1-C4 alkyl | C1-C2 haloalkyl | amino | CH2CH2 | Piperindinyl |
Cyclobutyl | C1-C3 alkyl
Pyrazolyl | C1-C4 alkyl | C1-C3 alkyl | C1-C2 haloalkyl | CH2 | Piperindinyl |
Pyrazolyl | C1-C3 alkyl
Imidazolyl | Benzyl | C1-C2 haloalkyl | C1-C3 alkyl | CH2CH2 | Piperindinyl |
Cyclobutyl | C1-C3 alkyl
Pyrazolyl | C1-C4 alkyl | Halogen | amino | CH2CH2 | Morpholinyl | Pyrazolyl | C1-C3
alkoxy
Pyridinyl | C1-C3 alkoxy | H | cyano | CH2CH2 | N(CH2CH3)2 | Pyridinyl | C1-C3
alkoxy
Pyrimidinyl | C1-C4 alkyl | C1-C3 alkyl | cyano | CH2CH2 | Piperazinyl | Imidazolyl |
C1-C3 haloalkyl
Pyrazolyl | Pyridinyl | C1-C3 alkyl | Halogen | CH2CH2 | N(CH2CH3)2 | Imidazolyl |
Halogen
Pyrazolyl | C1-C3 alkoxy | C1-C3 alkyl | H | CH2 | N(CH2CH3)2 | Cyclobutyl |
Halogen
Imidazolyl | Pyridinyl | amino | C1-C3 alkyl | CH2CH2 | Morpholinyl | Benzyl | C1-C3
alkoxy
Pyrazolyl | C1-C3 alkoxy | C1-C3 alkyl | amino | CH2 | N(CH3)2 | Cyclobutyl |
Halogen
Pyrazolyl | C1-C3 alkoxy | amino | cyano | CH2CH2 | Morpholinyl | Benzyl | Halogen
Pyridinyl | C1-C4 alkyl | amino | amino | CH2CH2 | N(CH3)2 | Imidazolyl | C1-C3
alkoxy
Pyrimidinyl | C1-C4 alkyl | amino | H | CH2 | N(CH2CH3)2 | Pyridinyl | C1-C3
haloalkyl
Imidazolyl | C1-C3 alkoxy | Halogen | H | CH2 | N(CH2CH3)2 | Cyclobutyl | C1-C3
alkyl
Imidazolyl | C1-C3 alkoxy | H | Halogen | CH2 | Piperindinyl | Benzyl | Halogen
Pyrimidinyl | Pyridinyl | amino | amino | CH2 | Piperindinyl | Pyrazolyl | C1-C3 alkyl
Imidazolyl | C1-C3 alkoxy | Halogen | C1-C3 alkyl | CH2CH2 | Piperazinyl | Benzyl |
C1-C3 alkoxy
Pyrimidinyl | Benzyl | C1-C2 haloalkyl | amino | CH2CH2 | N(CH3)2 | Pyrazolyl | C1-
C3 haloalkyl
Pyridinyl | Pyridinyl | amino | cyano | CH2CH2 | Piperindinyl | Benzyl | Halogen
Pyridinyl | C1-C3 alkoxy | C1-C2 haloalkyl | C1-C3 alkyl | CH2CH2 | Piperazinyl |
Cyclobutyl | C1-C3 haloalkyl
Imidazolyl | Pyridinyl | cyano | Halogen | CH2CH2 | N(CH3)2 | Pyridinyl | C1-C3 alkyl
Imidazolyl | C1-C3 alkoxy | amino | cyano | CH2CH2 | Morpholinyl | Cyclobutyl | C1-
C3 haloalkyl
Pyrazolyl | Benzyl | C1-C2 haloalkyl | Halogen | CH2 | N(CH2CH3)2 | Benzyl | C1-C3
alkoxy
Pyridinyl | C1-C3 alkoxy | C1-C3 alkyl | C1-C3 alkyl | CH2 | Morpholinyl | Cyclobutyl |
C1-C3 haloalkyl
Pyridinyl | C1-C3 alkoxy | amino | Halogen | CH2CH2 | N(CH2CH3)2 | Cyclobutyl |
Halogen
Imidazolyl | Benzyl | Halogen | H | CH2CH2 | Piperazinyl | Imidazolyl | Halogen
Pyridinyl | Benzyl | Halogen | Halogen | CH2 | N(CH3)2 | Benzyl | C1-C3 haloalkyl
Pyrazolyl | C1-C3 alkoxy | cyano | H | CH2 | N(CH3)2 | Pyrazolyl | C1-C3 alkoxy
Imidazolyl | Benzyl | cyano | C1-C3 alkyl | CH2 | N(CH2CH3)2 | Pyridinyl | C1-C3
alkyl
Imidazolyl | C1-C4 alkyl | C1-C3 alkyl | H | CH2 | Piperindinyl | Imidazolyl | Halogen
Pyridinyl | C1-C4 alkyl | H | cyano | CH2 | Morpholinyl | Imidazolyl | C1-C3 alkoxy
Pyrazolyl | Benzyl | H | C1-C2 haloalkyl | CH2CH2 | N(CH3)2 | Imidazolyl | C1-C3
haloalkyl
Imidazolyl | Benzyl | C1-C2 haloalkyl | Halogen | CH2CH2 | Piperindinyl | Pyrazolyl |
C1-C3 alkyl
Imidazolyl | C1-C3 alkoxy | amino | amino | CH2 | N(CH2CH3)2 | Benzyl | C1-C3
alkoxy
Pyrimidinyl | Benzyl | Halogen | C1-C2 haloalkyl | CH2CH2 | Morpholinyl | Imidazolyl |
C1-C3 haloalkyl
Pyrazolyl | Pyridinyl | H | C1-C2 haloalkyl | CH2 | Piperindinyl | Pyridinyl | C1-C3
alkoxy
Imidazolyl | C1-C3 alkoxy | H | amino | CH2CH2 | Piperindinyl | Pyrazolyl | Halogen
Imidazolyl | Pyridinyl | cyano | Halogen | CH2 | Morpholinyl | Benzyl | C1-C3 alkoxy
Pyridinyl | Pyridinyl | amino | H | CH2CH2 | N(CH2CH3)2 | Pyrazolyl | C1-C3 alkyl
Pyridinyl | C1-C3 alkoxy | C1-C3 alkyl | C1-C3 alkyl | CH2 | Piperindinyl | Imidazolyl |
C1-C3 haloalkyl
Imidazolyl | Benzyl | C1-C3 alkyl | C1-C2 haloalkyl | CH2CH2 | N(CH3)2 | Imidazolyl |
C1-C3 haloalkyl
Pyrimidinyl | C1-C4 alkyl | C1-C3 alkyl | amino | CH2 | Piperazinyl | Cyclobutyl | C1-
C3 haloalkyl
Pyridinyl | Pyridinyl | Halogen | H | CH2 | N(CH3)2 | Pyrazolyl | Halogen
Pyrazolyl | Benzyl | amino | C1-C3 alkyl | CH2CH2 | N(CH3)2 | Pyrazolyl | Halogen
Imidazolyl | C1-C4 alkyl | cyano | Halogen | CH2CH2 | N(CH2CH3)2 | Cyclobutyl |
Halogen
Pyrazolyl | Benzyl | C1-C3 alkyl | H | CH2CH2 | Piperindinyl | Pyrazolyl | C1-C3 alkyl
Pyrimidinyl | C1-C3 alkoxy | C1-C2 haloalkyl | amino | CH2CH2 | N(CH2CH3)2 |
Imidazolyl | C1-C3 alkyl
Imidazolyl | C1-C4 alkyl | amino | C1-C3 alkyl | CH2CH2 | Piperazinyl | Pyrazolyl |
C1-C3 alkoxy
Pyrazolyl | C1-C3 alkoxy | amino | Halogen | CH2 | N(CH2CH3)2 | Pyrazolyl | C1-C3
haloalkyl
Pyrazolyl | Pyridinyl | C1-C3 alkyl | H | CH2CH2 | Piperindinyl | Pyridinyl | C1-C3
haloalkyl
Pyridinyl | Benzyl | Halogen | Halogen | CH2CH2 | Piperazinyl | Pyrazolyl | Halogen
Pyridinyl | C1-C4 alkyl | H | C1-C2 haloalkyl | CH2CH2 | N(CH2CH3)2 | Benzyl | C1-
C3 alkyl
Imidazolyl | C1-C3 alkoxy | C1-C3 alkyl | amino | CH2 | N(CH2CH3)2 | Pyridinyl | C1-
C3 haloalkyl
Imidazolyl | C1-C4 alkyl | Halogen | Halogen | CH2CH2 | N(CH3)2 | Cyclobutyl |
Halogen
Pyrazolyl | Pyridinyl | amino | C1-C3 alkyl | CH2CH2 | Morpholinyl | Benzyl | Halogen
Imidazolyl | Pyridinyl | C1-C3 alkyl | C1-C3 alkyl | CH2CH2 | Piperazinyl | Pyrazolyl |
C1-C3 alkoxy
Imidazolyl | Pyridinyl | amino | cyano | CH2 | Piperindinyl | Imidazolyl | C1-C3 alkoxy
Pyrazolyl | C1-C4 alkyl | C1-C3 alkyl | C1-C3 alkyl | CH2 | Piperindinyl | Pyridinyl |
C1-C3 alkoxy
Pyrimidinyl | C1-C3 alkoxy | Halogen | H | CH2CH2 | N(CH3)2 | Pyridinyl | C1-C3
alkyl
Imidazolyl | C1-C3 alkoxy | Halogen | H | CH2CH2 | Piperindinyl | Pyrazolyl | Halogen
Pyrimidinyl | Pyridinyl | C1-C3 alkyl | C1-C2 haloalkyl | CH2 | N(CH2CH3)2 | Benzyl |
C1-C3 alkyl
Pyridinyl | Pyridinyl | amino | Halogen | CH2CH2 | N(CH2CH3)2 | Benzyl | C1-C3
alkoxy
Pyrimidinyl | Benzyl | C1-C2 haloalkyl | C1-C3 alkyl | CH2 | N(CH3)2 | Imidazolyl |
C1-C3 haloalkyl
Pyrazolyl | C1-C3 alkoxy | amino | C1-C2 haloalkyl | CH2 | Morpholinyl | Cyclobutyl |
C1-C3 haloalkyl
Pyridinyl | C1-C3 alkoxy | Halogen | cyano | CH2 | N(CH3)2 | Cyclobutyl | Halogen
Pyrimidinyl | C1-C3 alkoxy | H | amino | CH2 | N(CH3)2 | Pyridinyl | C1-C3 alkyl
Imidazolyl | C1-C4 alkyl | amino | C1-C2 haloalkyl | CH2 | Morpholinyl | Pyrazolyl |
C1-C3 alkoxy
Pyrazolyl | C1-C3 alkoxy | C1-C3 alkyl | cyano | CH2 | N(CH3)2 | Imidazolyl | Halogen
Pyrimidinyl | Pyridinyl | amino | H | CH2CH2 | N(CH2CH3)2 | Benzyl | C1-C3 alkoxy
Pyrimidinyl | C1-C3 alkoxy | cyano | Halogen | CH2CH2 | Piperindinyl | Cyclobutyl |
C1-C3 alkyl
Pyrimidinyl | C1-C3 alkoxy | cyano | H | CH2 | Piperazinyl | Imidazolyl | C1-C3 alkyl
Pyrimidinyl | Benzyl | amino | amino | CH2 | Morpholinyl | Imidazolyl | C1-C3
haloalkyl
Imidazolyl | Pyridinyl | Halogen | cyano | CH2 | Piperazinyl | Benzyl | C1-C3 alkyl
Pyrimidinyl | Pyridinyl | Halogen | C1-C2 haloalkyl | CH2CH2 | N(CH3)2 | Benzyl |
C1-C3 alkoxy
Pyrazolyl | Pyridinyl | amino | H | CH2CH2 | N(CH3)2 | Imidazolyl | C1-C3 alkyl
Imidazolyl | C1-C4 alkyl | Halogen | cyano | CH2CH2 | Piperindinyl | Benzyl | C1-C3
haloalkyl
Pyrazolyl | Pyridinyl | H | cyano | CH2 | Piperazinyl | Cyclobutyl | Halogen
Pyrazolyl | C1-C3 alkoxy | C1-C2 haloalkyl | amino | CH2 | N(CH3)2 | Pyrazolyl | C1-
C3 haloalkyl
Pyridinyl | C1-C4 alkyl | Halogen | cyano | CH2CH2 | Piperindinyl | Pyrazolyl |
Halogen
Pyrimidinyl | C1-C4 alkyl | C1-C3 alkyl | C1-C3 alkyl | CH2CH2 | N(CH2CH3)2 |
Pyridinyl | C1-C3 alkoxy
Pyrimidinyl | C1-C4 alkyl | cyano | cyano | CH2CH2 | Piperazinyl | Pyrazolyl | C1-C3
alkoxy
Pyrimidinyl | Benzyl | amino | amino | CH2 | Piperindinyl | Imidazolyl | C1-C3 alkyl
Pyrazolyl | C1-C3 alkoxy | H | H | CH2CH2 | Piperindinyl | Cyclobutyl | C1-C3 alkoxy
Pyridinyl | Benzyl | Halogen | C1-C2 haloalkyl | CH2CH2 | N(CH2CH3)2 | Pyrazolyl |
C1-C3 alkoxy
Pyridinyl | Pyridinyl | cyano | C1-C2 haloalkyl | CH2CH2 | Piperazinyl | Cyclobutyl |
Halogen
Pyrimidinyl | C1-C4 alkyl | amino | amino | CH2CH2 | Morpholinyl | Benzyl | Halogen
Pyridinyl | C1-C3 alkoxy | amino | C1-C2 haloalkyl | CH2 | N(CH2CH3)2 | Imidazolyl |
Halogen
Pyrimidinyl | Benzyl | cyano | H | CH2 | Piperindinyl | Pyrazolyl | C1-C3 alkoxy
Pyrimidinyl | C1-C4 alkyl | H | Halogen | CH2 | Piperazinyl | Pyrazolyl | C1-C3 alkoxy
Pyridinyl | C1-C3 alkoxy | C1-C3 alkyl | cyano | CH2CH2 | N(CH3)2 | Benzyl | C1-C3
alkoxy
Pyridinyl | Benzyl | Halogen | Halogen | CH2CH2 | N(CH3)2 | Pyrazolyl | C1-C3
alkoxy
Imidazolyl | C1-C4 alkyl | H | cyano | CH2CH2 | Piperindinyl | Imidazolyl | C1-C3
alkyl
Pyrazolyl | C1-C4 alkyl | C1-C3 alkyl | H | CH2 | Piperazinyl | Cyclobutyl | C1-C3 alkyl
Pyrimidinyl | Pyridinyl | Halogen | H | CH2CH2 | Piperazinyl | Imidazolyl | C1-C3 alkyl
Pyrimidinyl | C1-C3 alkoxy | cyano | C1-C3 alkyl | CH2 | Piperazinyl | Benzyl | C1-C3
alkoxy
Pyrimidinyl | C1-C4 alkyl | C1-C2 haloalkyl | C1-C3 alkyl | CH2CH2 | N(CH2CH3)2 |
Benzyl | C1-C3 alkyl
Pyrazolyl | Pyridinyl | Halogen | H | CH2 | N(CH3)2 | Pyridinyl | C1-C3 alkoxy
Pyrazolyl | C1-C3 alkoxy | Halogen | Halogen | CH2CH2 | Morpholinyl | Cyclobutyl |
C1-C3 haloalkyl
Imidazolyl | C1-C4 alkyl | H | amino | CH2CH2 | Morpholinyl | Benzyl | C1-C3 alkoxy
Pyrimidinyl | Benzyl | cyano | amino | CH2 | N(CH3)2 | Pyrazolyl | C1-C3 alkyl
Imidazolyl | Benzyl | C1-C3 alkyl | H | CH2 | Piperindinyl | Pyridinyl | C1-C3 alkyl
Pyrazolyl | Pyridinyl | cyano | C1-C2 haloalkyl | CH2 | N(CH2CH3)2 | Benzyl | C1-C3
haloalkyl
Imidazolyl | C1-C3 alkoxy | C1-C3 alkyl | Halogen | CH2CH2 | Piperindinyl | Pyridinyl |
C1-C3 alkyl
Pyrazolyl | Benzyl | C1-C3 alkyl | Halogen | CH2CH2 | Piperindinyl | Benzyl | C1-C3
haloalkyl
Pyridinyl | Benzyl | amino | Halogen | CH2 | Piperindinyl | Imidazolyl | C1-C3
haloalkyl
Pyridinyl | Benzyl | H | C1-C2 haloalkyl | CH2 | Piperindinyl | Benzyl | C1-C3 alkoxy
Pyrimidinyl | Benzyl | H | cyano | CH2CH2 | N(CH2CH3)2 | Imidazolyl | C1-C3 alkoxy
Pyrazolyl | C1-C4 alkyl | Halogen | amino | CH2CH2 | Piperindinyl | Imidazolyl |
Halogen
Imidazolyl | C1-C4 alkyl | H | C1-C2 haloalkyl | CH2CH2 | N(CH2CH3)2 | Imidazolyl |
Pyridinyl | C1-C4 alkyl | cyano | Halogen | CH2CH2 | Piperazinyl | Benzyl | C1-C3
haloalkyl
Imidazolyl | C1-C4 alkyl | H | Halogen | CH2 | Piperazinyl | Pyrazolyl | C1-C3 alkoxy
Pyridinyl | Benzyl | C1-C3 alkyl | C1-C2 haloalkyl | CH2CH2 | Piperindinyl | Pyridinyl |
Halogen
Imidazolyl | C1-C3 alkoxy | C1-C3 alkyl | C1-C3 alkyl | CH2 | Piperazinyl | Pyrazolyl |
C1-C3 alkoxy
Pyrimidinyl | C1-C4 alkyl | amino | cyano | CH2 | Piperindinyl | Imidazolyl | C1-C3
alkoxy
Pyridinyl | Pyridinyl | C1-C3 alkyl | C1-C3 alkyl | CH2CH2 | N(CH2CH3)2 |
Cyclobutyl | C1-C3 alkoxy
Imidazolyl | C1-C4 alkyl | C1-C3 alkyl | Halogen | CH2CH2 | N(CH2CH3)2 | Pyrazolyl |
C1-C3 alkyl
Pyrimidinyl | C1-C4 alkyl | C1-C3 alkyl | Halogen | CH2 | Morpholinyl | Cyclobutyl |
Halogen
Pyrimidinyl | C1-C4 alkyl | amino | amino | CH2CH2 | N(CH2CH3)2 | Pyridinyl |
Halogen
Imidazolyl | C1-C3 alkoxy | cyano | C1-C3 alkyl | CH2CH2 | Piperindinyl | Benzyl |
C1-C3 alkyl
Imidazolyl | C1-C3 alkoxy | cyano | cyano | CH2CH2 | Piperazinyl | Pyridinyl | C1-C3
alkyl
Pyridinyl | C1-C4 alkyl | amino | amino | CH2 | Piperindinyl | Benzyl | C1-C3 haloalkyl
Pyrazolyl | Pyridinyl | C1-C2 haloalkyl | Halogen | CH2 | N(CH3)2 | Pyridinyl | C1-C3
alkyl
Pyrimidinyl | C1-C3 alkoxy | C1-C3 alkyl | C1-C2 haloalkyl | CH2CH2 | Piperazinyl |
Imidazolyl | Halogen
Pyrazolyl | Pyridinyl | Halogen | amino | CH2CH2 | Morpholinyl | Cyclobutyl | Halogen
Pyrazolyl | C1-C3 alkoxy | Halogen | Halogen | CH2 | Piperazinyl | Benzyl | C1-C3
haloalkyl
Pyridinyl | Pyridinyl | Halogen | C1-C2 haloalkyl | CH2CH2 | Morpholinyl | Pyridinyl |
C1-C3 haloalkyl
Pyrazolyl | C1-C4 alkyl | H | H | CH2 | N(CH2CH3)2 | Imidazolyl | C1-C3 alkoxy
Pyridinyl | C1-C3 alkoxy | C1-C3 alkyl | C1-C3 alkyl | CH2CH2 | N(CH2CH3)2 |
Pyridinyl | Halogen
Imidazolyl | C1-C3 alkoxy | cyano | H | CH2CH2 | N(CH2CH3)2 | Pyridinyl | C1-C3
alkyl
Pyrimidinyl | C1-C4 alkyl | amino | C1-C2 haloalkyl | CH2CH2 | Piperindinyl |
Pyrazolyl | C1-C3 alkoxy
Pyridinyl | C1-C4 alkyl | C1-C3 alkyl | Halogen | CH2CH2 | N(CH3)2 | Imidazolyl |
C1-C3 haloalkyl
Imidazolyl | C1-C3 alkoxy | C1-C3 alkyl | C1-C3 alkyl | CH2CH2 | Morpholinyl |
Pyrazolyl | Halogen
Imidazolyl | C1-C4 alkyl | cyano | Halogen | CH2CH2 | Piperindinyl | Benzyl | C1-C3
alkyl
Imidazolyl | Benzyl | C1-C3 alkyl | C1-C3 alkyl | CH2CH2 | Morpholinyl | Benzyl | C1-
C3 alkoxy
Pyrimidinyl | C1-C3 alkoxy | C1-C3 alkyl | C1-C2 haloalkyl | CH2 | N(CH2CH3)2 |
Cyclobutyl | Halogen
Pyrazolyl | C1-C4 alkyl | Halogen | C1-C2 haloalkyl | CH2 | N(CH2CH3)2 | Cyclobutyl |
C1-C3 alkoxy
Pyrazolyl | C1-C4 alkyl | H | amino | CH2CH2 | Piperindinyl | Cyclobutyl | C1-C3
alkoxy
Pyridinyl | Benzyl | H | cyano | CH2 | Piperazinyl | Cyclobutyl | C1-C3 haloalkyl
Pyridinyl | Pyridinyl | cyano | C1-C3 alkyl | CH2CH2 | N(CH3)2 | Pyridinyl | C1-C3
haloalkyl
Imidazolyl | Pyridinyl | H | C1-C3 alkyl | CH2CH2 | Morpholinyl | Benzyl | C1-C3
alkyl
Pyrimidinyl | C1-C4 alkyl | Halogen | H | CH2 | N(CH3)2 | Pyridinyl | C1-C3 haloalkyl
Pyrimidinyl | Benzyl | amino | Halogen | CH2 | Piperazinyl | Benzyl | Halogen
Pyridinyl | C1-C3 alkoxy | C1-C3 alkyl | Halogen | CH2 | N(CH2CH3)2 | Imidazolyl |
C1-C3 alkyl
Imidazolyl | C1-C4 alkyl | H | cyano | CH2 | Morpholinyl | Pyridinyl | Halogen
Pyrimidinyl | C1-C3 alkoxy | C1-C2 haloalkyl | H | CH2CH2 | Piperazinyl | Pyridinyl |
C1-C3 haloalkyl
Pyrimidinyl | C1-C3 alkoxy | amino | Halogen | CH2CH2 | Morpholinyl | Benzyl | C1-
C3 haloalkyl
Pyrimidinyl | C1-C4 alkyl | C1-C3 alkyl | amino | CH2CH2 | N(CH2CH3)2 | Pyrazolyl |
C1-C3 haloalkyl
Pyrazolyl | Pyridinyl | amino | Halogen | CH2CH2 | N(CH3)2 | Pyridinyl | C1-C3
alkoxy
Imidazolyl | C1-C3 alkoxy | amino | C1-C3 alkyl | CH2 | N(CH2CH3)2 | Imidazolyl |
Halogen
Pyrazolyl | Benzyl | C1-C3 alkyl | Halogen | CH2CH2 | Piperindinyl | Cyclobutyl | C1-
C3 haloalkyl
Pyridinyl | C1-C3 alkoxy | C1-C2 haloalkyl | C1-C3 alkyl | CH2CH2 | Morpholinyl |
Benzyl | C1-C3 alkyl
Pyrazolyl | Benzyl | cyano | amino | CH2CH2 | Piperindinyl | Imidazolyl | C1-C3
haloalkyl
Pyridinyl | C1-C4 alkyl | C1-C2 haloalkyl | amino | CH2CH2 | N(CH2CH3)2 | Benzyl |
Halogen
Pyrazolyl | Pyridinyl | C1-C2 haloalkyl | Halogen | CH2 | Piperazinyl | Benzyl | C1-C3
alkoxy
Pyridinyl | C1-C4 alkyl | cyano | Halogen | CH2 | Piperazinyl | Pyrazolyl | C1-C3
haloalkyl
Imidazolyl | C1-C3 alkoxy | C1-C2 haloalkyl | C1-C3 alkyl | CH2 | Piperazinyl |
Imidazolyl | C1-C3 haloalkyl
Imidazolyl | Pyridinyl | H | C1-C3 alkyl | CH2CH2 | N(CH3)2 | Pyrazolyl | Halogen
Imidazolyl | C1-C4 alkyl | C1-C3 alkyl | C1-C2 haloalkyl | CH2CH2 | Morpholinyl |
Imidazolyl | C1-C3 alkyl

We claim:

1. A compound having the structure of Formula I: 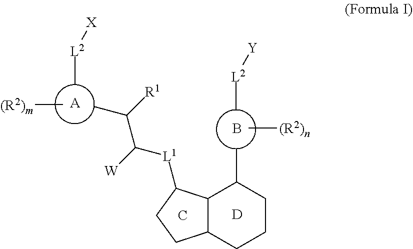
or a pharmaceutically acceptable salt thereof,
wherein:
A is aryl or heteroaryl;
B is a bond, aryl or heteroaryl;
the 5,6-membered bicyclic heteroaryl represented by C and D is selected from: 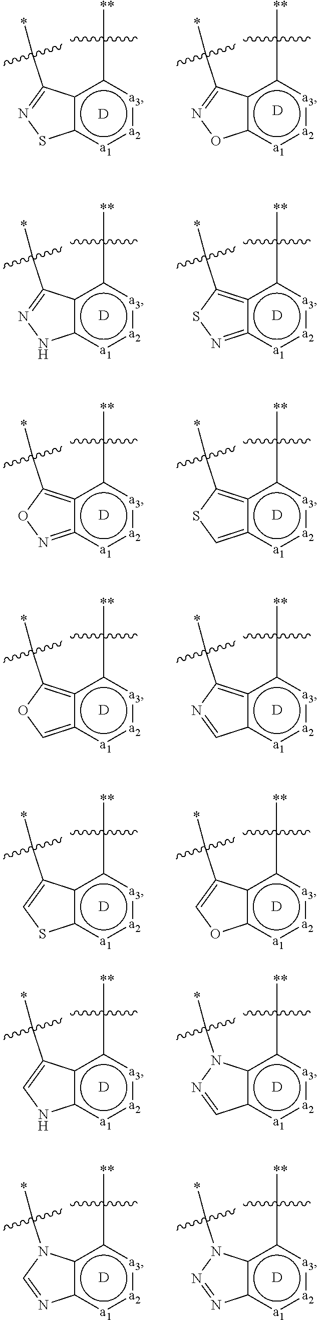 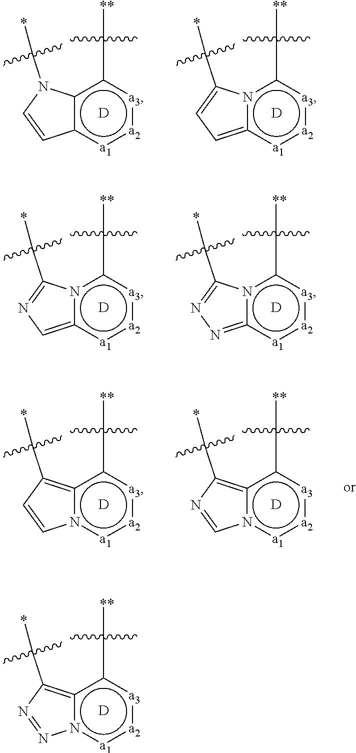
where [symbol] represents the points of attachment, * represents the point attaching to L¹, and ** represents the point attaching to B;
a₁ is CH, N or NH;
a₂ is C(Z¹), N or N(Z²);
a₃ is C(Z¹), N or N(Z²),
provided that a₁, a₂, and as are selected such that ring D is aromatic;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
W is C(O)OR^{w1}, C(O)N(H)S(O)₂R^{W2}, S(O)₂N(H)C(O)R^{W2}, S(O)₂N(H)R^{W3}, 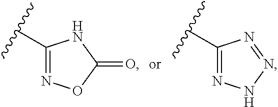 [SMILES: *c1noc(=O)[nH]1 ; *c1nn[nH]n1]
where [symbol] represents the point of attachment;
X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};
Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X2}), or hydroxyl, wherein each of said aryl, heteroaryl, cycloalkyl and heterocyclyl is optionally substituted with one, two, three or four R^{X3};
Z¹ is H, halogen, -L²-C_{y}, aryl, heteroaryl, cycloalkyl or heterocyclyl, wherein each of said aryl, heteroaryl, cycloalkyl and heterocyclyl is optionally substituted with one or more R^{Z1};
Z² is H, aryl, heteroaryl, cycloalkyl or heterocyclyl, wherein each of said aryl, heteroaryl, cycloalkyl and heterocyclyl is optionally substituted with one or more R^{Z1};
C_{y} is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 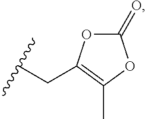 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W2} is C₁-C₆ alkyl, cycloalkyl, heterocyclyl, aryl or heteroaryl;
R^{W3} is aryl or heteroaryl;
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or
R^{X1} and R^{X2}, together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4, with the proviso that n is 0 when B is a bond; and
p in each instance is independently 0 or 1.

2. The compound of claim 1, or a pharmaceutically acceptable salt thereof, wherein:
A is aryl or heteroaryl;
B is aryl or heteroaryl;
the 5,6-membered bicyclic heteroaryl represented by C and D is selected from: 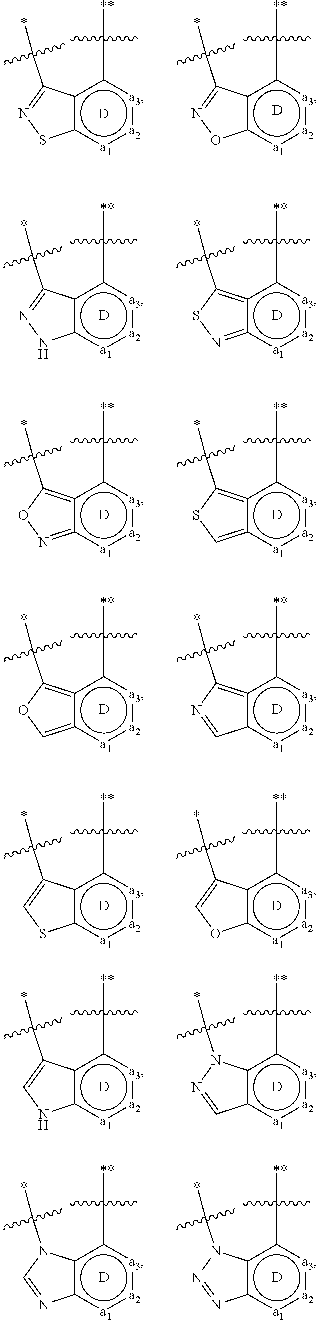 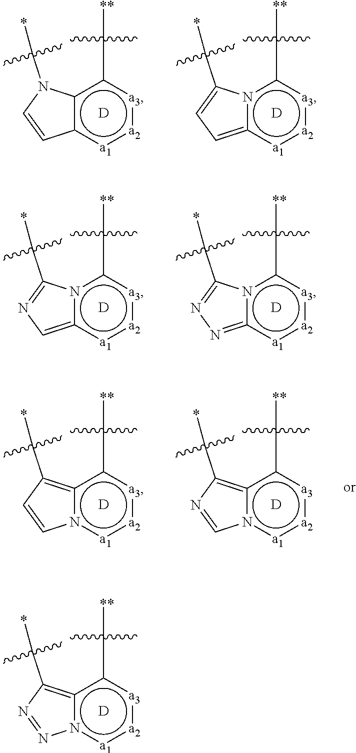
where [symbol] represents the points of attachment, * represents the point attaching to L¹, and ** represents the point attaching to B;
a₁ is CH, N or NH;
a₂ is C(Z¹), N or N(Z²);
a₃ is C(Z¹), N or N(Z²),
provided that a₁, a₂, and as are selected such that ring D is aromatic;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
W is C(O)OR^{W1}, C(O)N(H)S(O)₂R^{W2}, S(O)₂N(H)C(O)R^{W2}, S(O)₂N(H)R^{W3}, 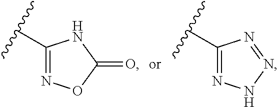 [SMILES: *c1noc(=O)[nH]1 ; *c1nn[nH]n1]
where [symbol] represents the point of attachment;
X is aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};
Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X2}), or hydroxyl, wherein each of said aryl, heteroaryl, cycloalkyl and heterocyclyl is optionally substituted with one, two, three or four R^{X3};
Z¹ is H, halogen, -L²-C_{y}, aryl, heteroaryl, cycloalkyl or heterocyclyl, wherein each of said aryl, heteroaryl, cycloalkyl and heterocyclyl is optionally substituted with one or more R^{Z1};
Z² is H, aryl, heteroaryl, cycloalkyl or heterocyclyl, wherein each of said aryl, heteroaryl, cycloalkyl and heterocyclyl is optionally substituted with one or more R^{Z1};
C_{y} is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W2} is C₁-C₆ alkyl, cycloalkyl, heterocyclyl, aryl or heteroaryl;
R^{W3} is aryl or heteroaryl;
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or
R^{X1} and R^{X2}, together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4; and
p in each instance is independently 0 or 1.

3. A compound having the structure of Formula II: 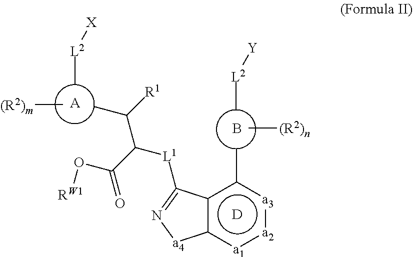
or a pharmaceutically acceptable salt thereof,
wherein:
A is aryl or heteroaryl;
B is a bond, aryl or heteroaryl;
a₁ is CH, N or NH;
a₂ is C(Z¹), N or N(Z²);
a₃ is C(Z¹), N or N(Z²);
a₄ is S, O or NH,
provided that a₁, a₂, and a₃ are selected such that ring D is aromatic;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
X is absent, aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};
Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X}), or hydroxyl, wherein each of said aryl, heteroaryl, cycloalkyl and heterocyclyl is optionally substituted with one, two, three or four R^{X3};
Z¹ is H, halogen, -L²-C_{y}, aryl, heteroaryl, cycloalkyl or heterocyclyl, wherein each of said aryl, heteroaryl, cycloalkyl and heterocyclyl is optionally substituted with one or more R^{Z1};
Z² is H, aryl, heteroaryl, cycloalkyl or heterocyclyl, wherein each of said aryl, heteroaryl, cycloalkyl and heterocyclyl is optionally substituted with one or more R^{Z1};
C_{y} is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 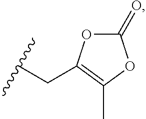 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or
R^{X1} and R^{X2}, together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4, with the proviso that n is 0 when B is a bond; and
p in each instance is independently 0 or 1.

4. The compound of claim 3, or a pharmaceutically acceptable salt thereof, wherein:
A is aryl or heteroaryl;
B is aryl or heteroaryl;
a₁ is CH, N or NH;
a₂ is C(Z¹), N or N(Z²);
a₃ is C(Z¹), N or N(Z²);
a₄ is S, O or NH,
provided that a₁, a₂, and as are selected such that ring D is aromatic;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ-S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
X is aryl, heteroaryl, cycloalkyl, heterocyclyl, C₁-C₆ haloalkyl, C₁-C₆ hydroxyalkyl, C₁-C₆ alkoxy, or N(R^{X1})(R^{X2}), wherein each of said aryl, heteroaryl, cycloalkyl and heterocyclyl is optionally substituted with one, two, three or four R^{X3};
Y is aryl, heteroaryl, cycloalkyl, heterocyclyl, C₁-C₆ haloalkyl, C₁-C₆ hydroxyalkyl, C₁-C₆ alkoxy, N(R^{X1})(R^{X2}), or hydroxyl, wherein each of said aryl, heteroaryl, cycloalkyl and heterocyclyl is optionally substituted with one, two, three or four R^{X3};
Z¹ is H, halogen, -L²-C_{y}, aryl, heteroaryl, cycloalkyl or heterocyclyl, wherein each of said aryl, heteroaryl, cycloalkyl and heterocyclyl is optionally substituted with one or more R^{Z1};
Z² is H, aryl, heteroaryl, cycloalkyl or heterocyclyl, wherein each of said aryl, heteroaryl, cycloalkyl and heterocyclyl is optionally substituted with one or more R^{Z1};
C_{y} is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R^{X2} are in each instance each independently H, C₁-C₆ alkyl, C₁-C₆ alkoxy, C₁-C₆ haloalkyl, C₁-C₆ hydroxyalkyl, aryl, heteroaryl, or heterocyclyl, wherein each of aryl, heteroaryl, or heterocyclyl are optionally substituted with one, two, three or four R^{X3}; or
R^{X1} and R^{X2}, together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4; and
p in each instance is independently 0 or 1.

5. A compound of any of claims 1-3, wherein A is phenyl, pyridine, pyridazine, pyrimidine, pyrazine or thiophene.

6. A compound of any of claims 1-5, wherein B is phenyl, pyridine or thiophene.

7. A compound of any of claims 1-6, wherein:
L¹ is O;
R¹ is H;
R² in each instance is independently C₁-C₆ alkyl or halogen;
m is 0; and
n is 2.

8. A compound of any of claims 1-7, wherein:
Y is 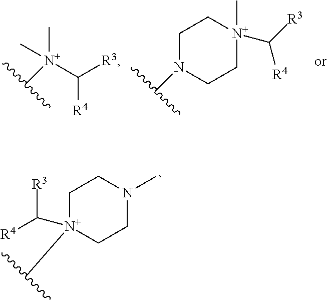
where [symbol] represents the point of attachment;
R³ is H or C₁-C₆ alkyl;
R⁴ is —O—P(O)(O⁻)(O⁻), —O—P(O)(O⁻)(OR⁵), —O—P(O)(OR⁵)(OR⁵), —O—S(O₂)—O⁻, —O—S(O₂)—OR⁵, Cy^{a}, —O—C(O)—R⁶, —O—C(O)—OR⁶, or —O—C(O)—N(R⁶)(R⁶);
Cy^{a} is cycloalkyl, heterocyclyl, aryl or heteroaryl;
R⁵ in each instance is independently H, C₁-C₆ alkyl, or aralkyl(C₁-C₆); and
R⁶ in each instance is independently H, C₁-C₆ alkyl, or C₁-C₆ aminoalkyl.

9. The compound of claim 3, wherein:
A is aryl;
B is aryl;
a₁ is CH;
a₂ is C(H) or N;
a₃ is C(Z¹);
a₄ is S, O or NH;
L¹ is a bond, CH₂, O, NH, S, SO, or SO₂;
L² in each instance is independently a bond or —(C₁-C₆ alkyl)ₚ—O—(C₁-C₆ alkyl)ₚ—;
X is aryl or heteroaryl, wherein each of said aryl and heteroaryl is optionally substituted with one, two, three or four R^{X3};
Y is heterocyclyl, C₁-C₆ hydroxyalkyl, C₁-C₆ alkoxy, N(R^{X1})(R^{X2}), or hydroxyl, wherein heterocyclyl is optionally substituted with one, two, three or four R^{X3};
Z¹ is aryl, heteroaryl, cycloalkyl or heterocyclyl, wherein each of said aryl, heteroaryl, cycloalkyl and heterocyclyl is optionally substituted with one or more R^{Z1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R^{X2} are in each instance each independently H, C₁-C₆ alkyl, C₁-C₆ alkoxy, C₁-C₆ haloalkyl or C₁-C₆ hydroxyalkyl; or
R^{X1} and R^{X2}, together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};
R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};
R^{Z3} in each instance is independently aryl, heteroaryl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
m is 0, 1, 2, or 3;
n is 0, 1, 2, 3 or 4; and
p in each instance is independently 0 or 1.

10. The compound of claim 3, or a pharmaceutically acceptable salt thereof, wherein:
A is aryl;
B is aryl;
a₁ is CH;
a₂ is C(Z¹);
a₃ is C(H);
a₄ is S, O or NH;
L¹ is a bond, CH₂, O, NH, S, SO, or SO₂;
L² in each instance is independently a bond or —(C₁-C₆ alkyl)ₚ—O—(C₁-C₆ alkyl)ₚ—;
X is aryl or heteroaryl, wherein each of said aryl and heteroaryl is optionally substituted with one, two, three or four R^{X3};
Y is heterocyclyl, C₁-C₆ hydroxyalkyl, C₁-C₆ alkoxy, N(R^{X1})(R^{X2}), or hydroxyl, wherein heterocyclyl is optionally substituted with one, two, three or four R^{X3};
Z¹ is aryl, heteroaryl, cycloalkyl or heterocyclyl, wherein each of said aryl, heteroaryl, cycloalkyl and heterocyclyl is optionally substituted with one or more R^{Z1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X1} and R² are in each instance each independently H, C₁-C₆ alkyl, C₁-C₆ alkoxy, C₁-C₆ haloalkyl or C₁-C₆ hydroxyalkyl; or
R^{X1} and R², together with the N to which they are connected, form a heterocycle or heteroaryl, wherein each of said heterocycle and heteroaryl is optionally substituted with one, two, three or four R^{X3};
R^{X3} in each instance is independently aryl, heteroaryl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one, two, three or four R^{X3}a;
R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;
R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};
R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};
R^{Z3} in each instance is independently aryl, heteroaryl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl and heteroaryl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
m is 0, 1, 2, or 3;
n is 0, 1, 2, 3 or 4; and
p in each instance is independently 0 or 1.

11. The compound of claim 10, or a pharmaceutically acceptable salt thereof, wherein:
Z¹ is an indazolyl, benzoimidazolyl, benzoimidazolonyl, indolyl, pyrrolopyridinyl or isoquinolinyl, wherein each of indazolyl, benzoimidazolyl, benzoimidazolonyl, indolyl, pyrrolopyridinyl and isoquinolinyl optionally substituted with one, two or three groups independently selected from C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano.

12. The compound of any one of claims 1-11, wherein L² is a bond and Y is hydroxyl.

13. The compound of any one of claims 1-12, wherein:
R^{W1} is H;
L¹ is O;
R¹ is H;
R² in each instance is independently C₁-C₆ alkyl or halogen;
m is 0; and
n is 2.

14. The compound of any one of claims 1-13, wherein a₂ is C(H) or N.

15. A compound having the structure of Formula III: 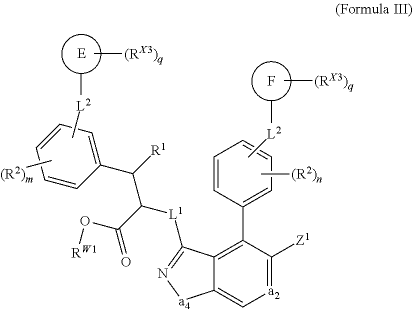
or a pharmaceutically acceptable salt thereof,
wherein:
a₂ is C(Z¹) or N;
a₄ is S, O or NH;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
E is absent, aryl, heteroaryl, cycloalkyl or heterocyclyl;
F is aryl, heteroaryl, cycloalkyl or heterocyclyl;
Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};
Cy is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 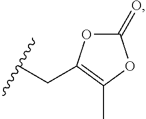 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4;
p in each instance is independently 0 or 1; and
q in each instance is independently 0, 1, 2, 3 or 4, with the proviso that q is 0 in —(R^{X3})_{q} when E is absent.

16. The compound of claim 15, or a pharmaceutically acceptable salt thereof, wherein:
a₂ is C(Z¹) or N;
a₄ is S, O or NH;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
E is aryl, heteroaryl, cycloalkyl or heterocyclyl;
F is aryl, heteroaryl, cycloalkyl or heterocyclyl;
Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};
Cy is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4;
p in each instance is independently 0 or 1; and
q in each instance is independently 0, 1, 2, 3 or 4.

17. The compound of claim 15, wherein:
a₂ is C(H) or N;
a₄ is S, O or NH;
L¹ is a bond, CH₂, O, NH, S, SO, or SO₂;
L² in each instance is independently a bond or —(C₁-C₆ alkyl)ₚ—O—(C₁-C₆ alkyl)ₚ—;
E is aryl or heteroaryl;
F is cycloalkyl or heterocyclyl;
Z¹ is aryl, heteroaryl, cycloalkyl or heterocyclyl, wherein each of said aryl, heteroaryl, cycloalkyl and heterocyclyl is optionally substituted with one or more R^{Z1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or, when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, nitro or cyano, wherein each of said C₁-C₆ alkyl, C₁-C₆ aminoalkyl and C₁-C₆ alkoxy is optionally substituted with one or more R^{Z3};
R^{Z2} in each instance is independently H, C₁-C₆ alkyl, C₁-C₆ alkoxy, C₁-C₆ haloalkyl or C₁-C₆ hydroxyalkyl, wherein each of said C₁-C₆ alkyl and C₁-C₆ alkoxy is optionally substituted with one or more R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
m is 0, 1, 2 or 3;
n is 0, 1, 2, 3 or 4;
p in each instance is independently 0 or 1; and
q in each instance is independently 0, 1, 2, 3 or 4.

18. The compound of claim 15, wherein:
R^{W1} is H;
L¹ is O;
R¹ is H;
R² in each instance is independently C₁-C₆ alkyl or halogen;
m is 0; and
n is 2.

19. The compound of claim 15, wherein a₂ is C(H) or N.

20. The compound of claim 15, wherein:
R¹ is H;
R² in each instance is independently C₁-C₆ alkyl or halogen;
m is 0; and
n is 2.

21. The compound of claim 15 wherein:
L¹ is O;
R¹ is H;
R² in each instance is independently C₁-C₆ alkyl or halogen;
m is 0; and
n is 2.

22. The compound of any one of claims 15-21, wherein Z¹ in each instance is independently H or halogen.

23. The compound of any one of claims 5-21, wherein Z¹ in each instance is independently H, Br or Cl.

24. The compound of any one of claims 5-21, wherein:
Z¹ in each instance is independently H, phenyl, pyridine, thiophene, furan, pyrrole, cyclopropyl, or cyclobutyl, wherein each of phenyl, pyridine, thiophene, furan, pyrrole, cyclopropyl, and cyclobutyl is optionally substituted with one or two R^{Z1}; and
R^{Z1} in each instance is independently halogen or C₁-C₆ alkyl.

25. The compound of claim 24, wherein R^{Z1} in each instance is independently methyl, ethyl, F or Cl.

26. A compound having the structure of Formula IV: 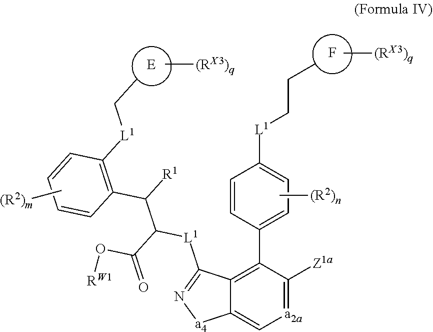
or a pharmaceutically acceptable salt thereof,
wherein:
a₂a is CH or N;
a₄ is S, O or NH;
L¹ in each instance is independently a bond, CH₂, O, NH, S, SO, or SO₂;
E is absent, aryl, heteroaryl, cycloalkyl or heterocyclyl;
F is aryl, heteroaryl, cycloalkyl or heterocyclyl;
Z^{1a} is H, halogen, aryl, heteroaryl, cycloalkyl or heterocyclyl, wherein each of said aryl, heteroaryl, cycloalkyl and heterocyclyl is optionally substituted with one or more R^{Z1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 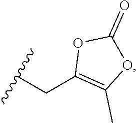 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
m is 0, 1, 2, or 3;
n is 0, 1, 2, 3 or 4; and
q in each instance is independently 0, 1, 2, 3 or 4, with the proviso that q is 0 in —(R^{X3})_{q} when E is absent.

27. The compound of claim 26, or a pharmaceutically acceptable salt thereof, wherein:
a₂a is CH or N;
a₄ is S, O or NH;
L¹ in each instance is independently a bond, CH₂, O, NH, S, SO, or SO₂;
E is aryl, heteroaryl, cycloalkyl or heterocyclyl;
F is aryl, heteroaryl, cycloalkyl or heterocyclyl;
Z^{1a} is H, halogen, aryl, heteroaryl, cycloalkyl or heterocyclyl, wherein each of said aryl, heteroaryl, cycloalkyl and heterocyclyl is optionally substituted with one or more R^{Z1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, or heteroaryl, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}) amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
m is 0, 1, 2, or 3;
n is 0, 1, 2, 3 or 4; and
q in each instance is independently 0, 1, 2, 3 or 4.

28. The compound of claim 26, wherein:
R¹ is H;
R² in each instance is independently C₁-C₆ alkyl or halogen;
m is 0; and
n is 2.

29. The compound of any one of claims 26-28, wherein Z^{1a} is halogen.

30. The compound of claim 27, wherein the halogen is Br or Cl.

31. The compound of any one of claims 26-28, wherein:
Z^{1a} is phenyl, pyridine, thiophene, furan, pyrrole, cyclopropyl, or cyclobutyl, wherein each is optionally substituted with one or two R^{Z1}; and
R^{Z1} in each instance is independently halogen or C₁-C₆ alkyl.

32. The compound of claim 31, wherein R^{Z1} in each instance is independently methyl, ethyl, F or Cl.

33. The compound of any of claims 15-32, wherein:
E is phenyl, pyridine, pyridazine, pyrimidine, pyrazine, triazine, furan, thiophene, pyrrole, pyrazole, imidazole or triazole;
F is pyrrolidine, piperidine, piperazine, tetrahydropyran, morpholine, 2,6-diazaspiro[3.3]heptanyl, 2-oxa-6-azaspiro[3.3]heptanyl, 2-azaspiro[3.3]heptanyl, or 2-oxaspiro[3.3]heptanyl;
R^{X3} in each instance is independently C₁-C₆ alkyl, C₁-C₆ alkoxy, C₁-C₆ haloalkyl, C₁-C₆ hydroxyalkyl, hydroxy, halogen, amino, nitro or cyano; and
q in each instance is independently 0, 1 or 2.

34. A compound having the structure of Formula V: 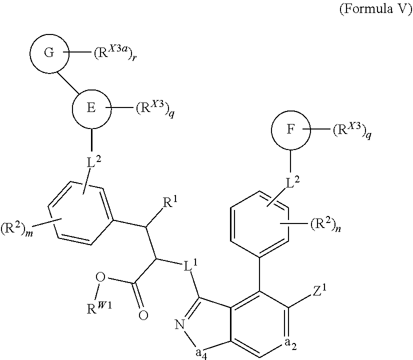
or a pharmaceutically acceptable salt thereof,
wherein:
a₂ is C(Z¹) or N;
a₄ is S, O or NH;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
E is aryl, heteroaryl, cycloalkyl or heterocyclyl;
F is aryl, heteroaryl, cycloalkyl or heterocyclyl;
G is aryl, heteroaryl, cycloalkyl or heterocyclyl;
Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};
Cy is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 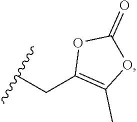 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4;
p in each instance is independently 0 or 1;
q in each instance is independently 0, 1, 2, 3 or 4; and
r is 0, 1, 2, 3 or 4.

35. A compound having the structure of Formula VI: 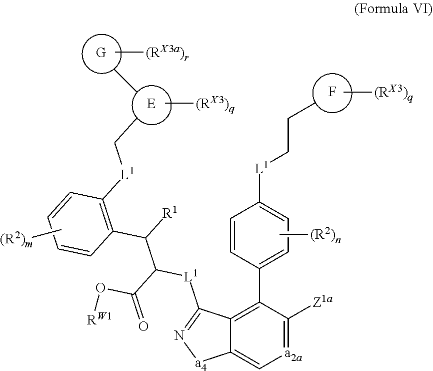
or a pharmaceutically acceptable salt thereof,
wherein:
a₂ is C(Z¹) or N;
a₄ is S, O or NH;
L¹ in each instance is independently a bond, CH₂, O, NH, S, SO, or SO₂;
E is aryl, heteroaryl, cycloalkyl or heterocyclyl;
F is aryl, heteroaryl, cycloalkyl or heterocyclyl;
G is aryl, heteroaryl, cycloalkyl or heterocyclyl;
Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};
Cy is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 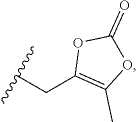 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4;
q in each instance is independently 0, 1, 2, 3 or 4; and
r is 0, 1, 2, 3 or 4.

36. A compound having the structure of Formula VII: 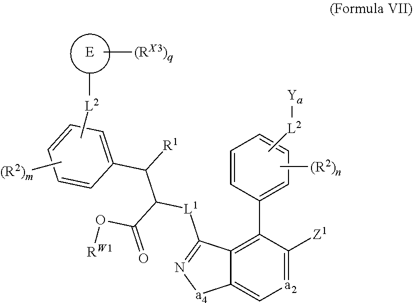
or a pharmaceutically acceptable salt thereof,
wherein:
a₂ is C(Z¹) or N;
a₄ is S, O or NH;
L¹ is a bond, CH₂, O, NH, S, SO, or SO₂;
L² in each instance is independently a bond, optionally substituted C₁-C₆ alkyl, —(C₁-C₆ alkyl)ₚ—O—(C₁-C₆ alkyl)ₚ—, —(C₁-C₆ alkyl)ₚ-N(R^{X1})—(C₁-C₆ alkyl)ₚ—, —(C₁-C₆ alkyl)ₚ—S—(C₁-C₆ alkyl)ₚ—, —(C₁-C₆ alkyl)ₚ—S(O)—(C₁-C₆ alkyl)ₚ—, or —(C₁-C₆ alkyl)ₚ—S(O)₂—(C₁-C₆ alkyl)ₚ—;
E is absent, aryl, heteroaryl, cycloalkyl or heterocyclyl;
Yₐ is C₁-C₆ hydroxyalkyl, C₁-C₆ alkoxy, N(R^{X1})(R^{X2}), or hydroxyl;
Z¹ in each instance is independently H, halogen, -L²-Cy, aryl, heteroaryl, cycloalkyl or heterocyclyl, wherein each of said aryl, heteroaryl, cycloalkyl and heterocyclyl is optionally substituted with one or more R^{Z1};
Cy is aryl, heteroaryl, cycloalkyl or heterocyclyl, wherein each is optionally substituted with one, two, three or four R^{Cy1};
R¹ is H, hydroxy, C₁-C₃ alkyl, C₁-C₂ haloalkyl, C₁-C₂ hydroxyalkyl, or C₁-C₂ alkoxy;
R² in each instance is independently cyano, halogen, hydroxy, C₁-C₆ alkyl, C₁-C₆ alkylamino, C₁-C₆ aminoalkyl, C₁-C₆ haloalkyl, C₁-C₆ hydroxyalkyl, C₁-C₆ alkoxy, nitro or N(R^{2a})(R^{2b});
R^{2a} and R^{2b} are each independently H, C₁-C₆ alkyl, C₁-C₆ haloalkyl, or C₁-C₆ hydroxyalkyl;
R^{W1} is H, C₁-C₆ alkyl, CH(R^{W1a})(R^{W2a}), heterocyclyl, aryl, heteroaryl, or 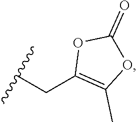 [SMILES: *Cc1oc(=O)oc1C]
where [symbol] represents the point of attachment, wherein each of said heterocyclyl, aryl and heteroaryl is optionally substituted with one, two, three or four R^{W3a};
R^{W1a} is H or C₁-C₆ alkyl;
R^{W2a} is OC(O)OR^{W2b}, OC(O)N(R^{W2b})(R^{W2b}) or OP(O)(OR^{W2b})₂;
R^{W2b} in each instance is independently H, C₁-C₆ alkyl, cycloalkyl or C₁-C₆ alkoxy; or,
when R^{W2a} is OC(O)N(R^{W2b})(R^{W2b}), the two R^{W2b}, together with the N to which they are connected, form a heterocyclyl or heteroaryl, wherein each of said heterocyclyl and heteroaryl is optionally substituted with one, two, three or four R^{W3a};
R^{W3a} is C₁-C₆ alkyl, C₁-C₆ alkoxy, C₁-C₆ haloalkyl, C₁-C₆ hydroxyalkyl, hydroxy, halogen, amino, nitro or cyano;
R^{X3} in each instance is independently aryl, heteroaryl, cycloalkyl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one, two, three or four R^{X3a};
R^{X3a} in each instance is independently heteroaryl, heterocyclyl, C₁-C₆ alkyl, C₁-C₆ alkoxy, C₁-C₆ haloalkyl, C₁-C₆ hydroxyalkyl, hydroxy, halogen, —C(O)N(R^{X3c})(R^{X3d}), amino, nitro, sulfonamide, sulfoxide, sulfonyl, or cyano, wherein each of heteroaryl, heterocyclyl, amino, nitro, sulfonamide, sulfoxide, sulfonyl, C₁-C₆ alkyl, C₁-C₆ alkoxy and C₁-C₆ hydroxyalkyl is optionally substituted with one, or two R^{X3b};
R^{X3b} in each instance is independently aryl, heteroaryl, heterocyclyl, C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano, wherein each of aryl, heteroaryl and heterocyclyl is optionally substituted with one, two, three or four groups each independently selected from C₁-C₆ alkyl, C₁-C₆ alkoxy, C₁-C₆ haloalkyl, C₁-C₆ hydroxyalkyl, C₁-C₆ acyl, halogen, amino, nitro, sulfonamide, sulfoxide, sulfonyl or cyano;
R^{X3c} and R^{X3d} is each independently selected from H, C₁-C₆ cycloalkyl, C₁-C₆ alkyl, C₁-C₆ alkoxy, C₁-C₆ haloalkyl, C₁-C₆ hydroxyalkyl, C₁-C₆ acyl; or
R^{X3c} and R^{X3d}, together with the N to which they are connected, form a 4-6 membered heterocycle optionally substituted with one or two groups each independently selected from C₁-C₆ alkyl, C₁-C₆ alkoxy, C₁-C₆ haloalkyl, C₁-C₆ hydroxyalkyl, C₁-C₆ acyl or halogen;R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano;
R^{Cy1} is aryl, heteroaryl, cycloalkyl, heterocyclyl, halogen, hydroxy, N(R^{Cy2})(R^{Cy2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano;
R^{Cy2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl;
m is 0, 1, 2, or 3;
n is 0, 1, 2, 3 or 4;
p in each instance is independently 0 or 1; and
q in each instance is independently 0, 1, 2, 3 or 4, with the proviso that q is 0 in —(R^{X3})_{q} when E is absent.

37. The compound of claim 36, or a pharmaceutically acceptable salt thereof, wherein:
E is phenyl, pyridine, pyridazine, pyrimidine, pyrazine, triazine, furan, thiophene, pyrrole, pyrazole, imidazole or triazole;
Yₐ is N(R^{X1})(R^{X2}); and
q in each instance is independently 0, 1 or 2.

38. The compound of any of claims 15, 26, 34, and 35, wherein F is 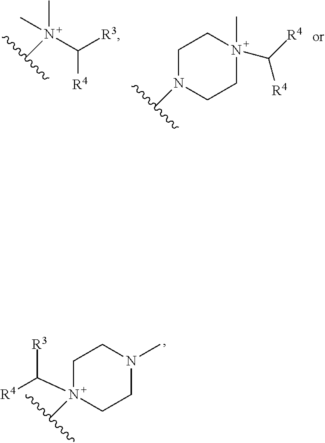
where [symbol] represents the point of attachment;
R₃ is H or C₁-C₆ alkyl;
R⁴ is —O—P(O)(O⁻)(O⁻), —O—P(O)(O⁻)(OR⁵),—O—P(O)(OR⁵)(OR⁵), —O—S(O₂)—O—,—O—S(O₂)—OR⁵, Cy^{a}, —O—C(O)—R⁶, —O—C(O)—OR⁶, or —O—C(O)—N(R⁶)(R⁶);
R⁵ in each instance is independently H, C₁-C₆ alkyl, or aralkyl(C₁-C₆);
R⁶ in each instance is independently H, C₁-C₆ alkyl, or C₁-C₆ aminoalkyl; and
q, in the instance of F substitution with R^{X3}, is 0.

39. The compound of any of claims 1, 3, 15, 26, 34, 35 and 36, wherein R^{W1} is

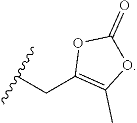 [SMILES: *Cc1oc(=O)oc1C]

40. The compound of any of claims 1-39, wherein C₁-C₆ haloalkyl is trifluoromethane or trifluoroethane.

41. A compound selected from: 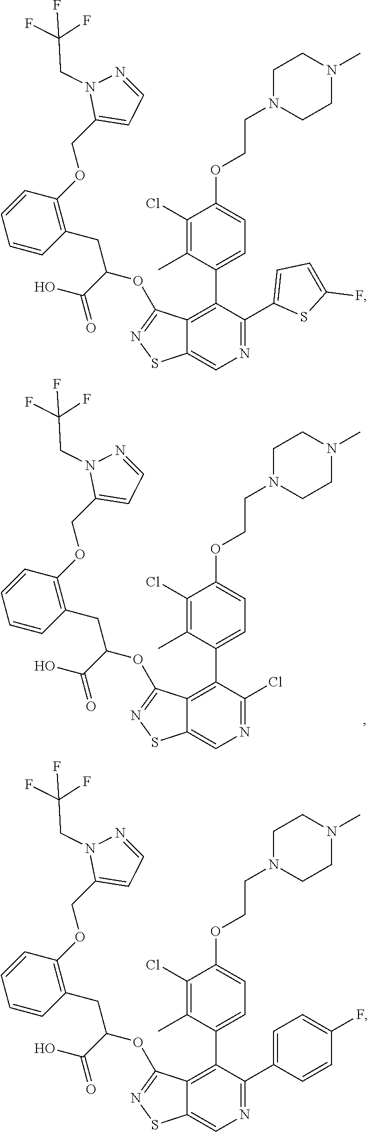 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl] 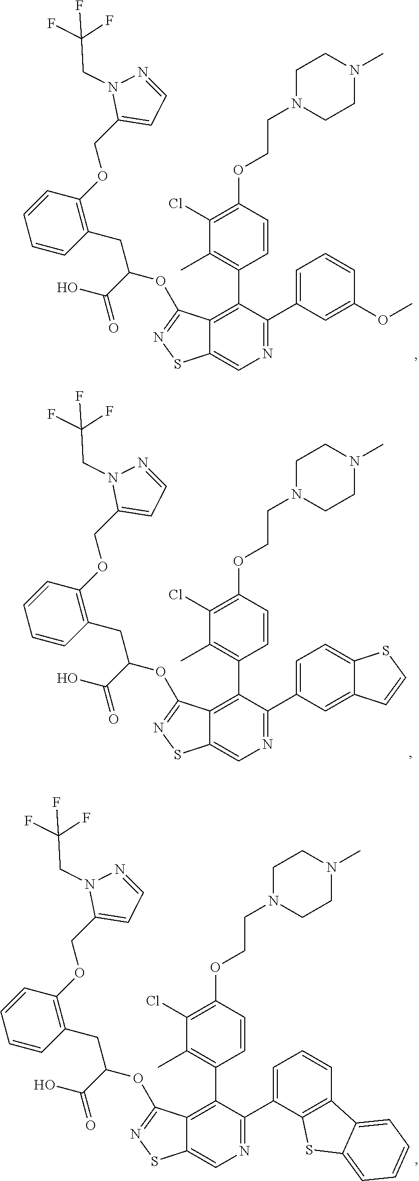 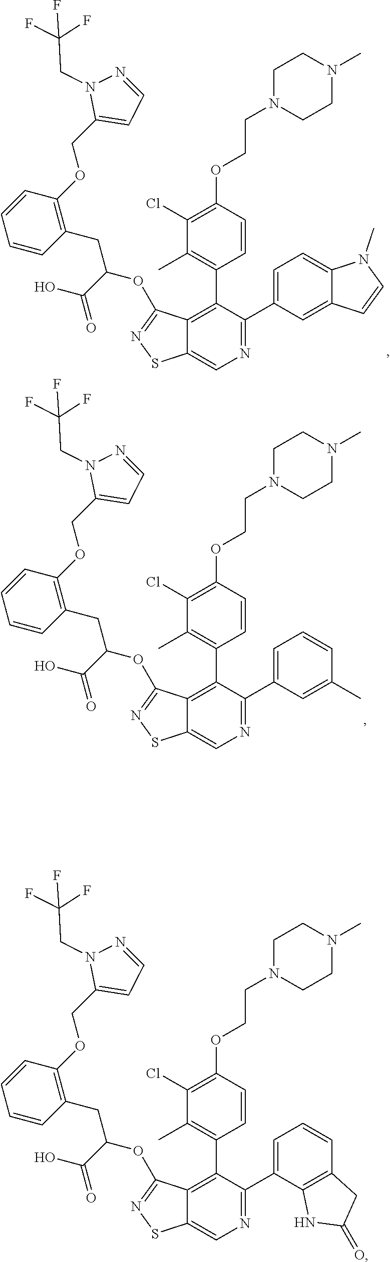 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl] 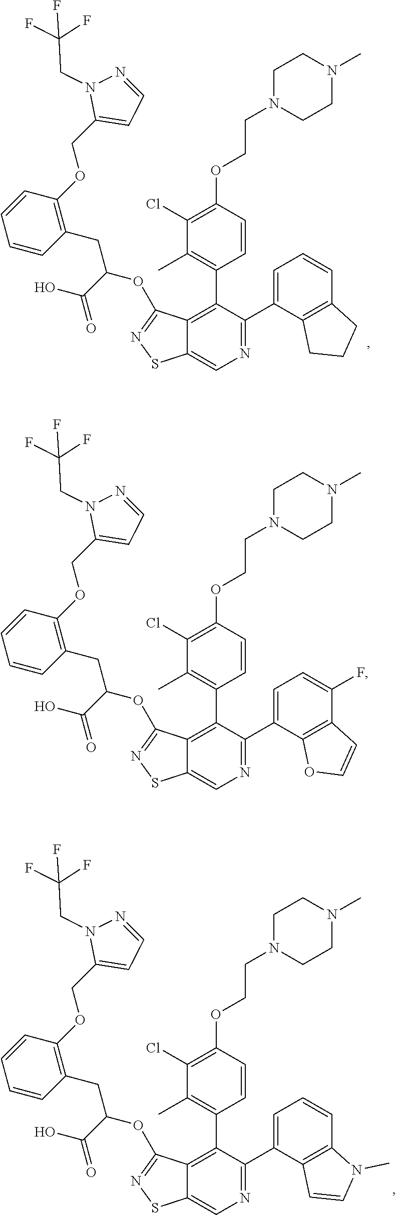 [SMILES: Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl] 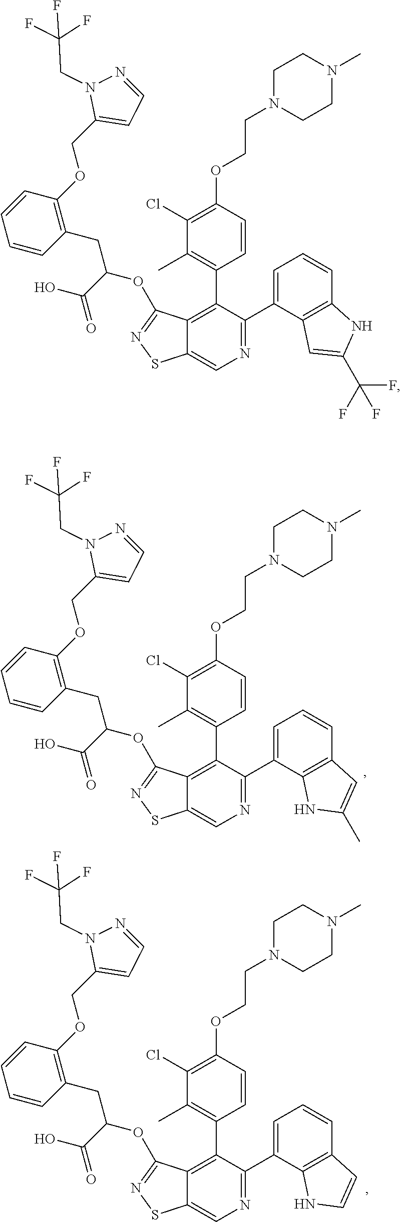 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1 ; Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl] 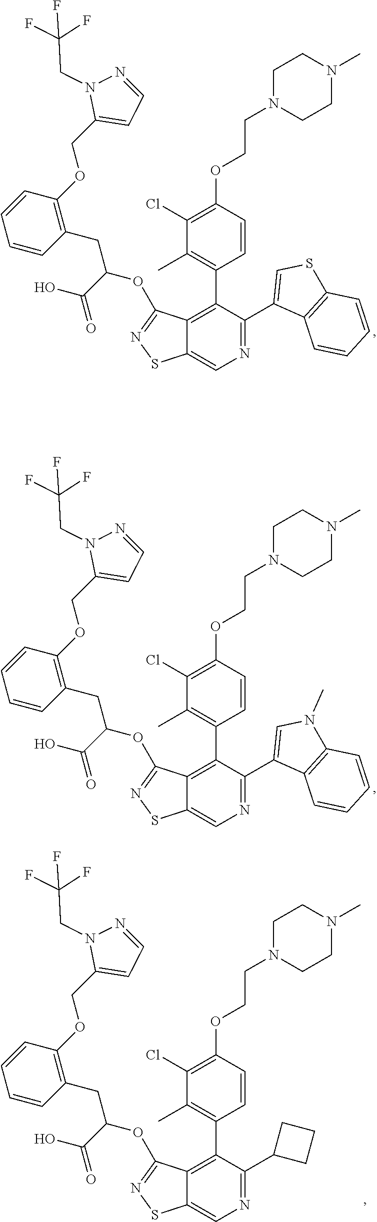 [SMILES: Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl] 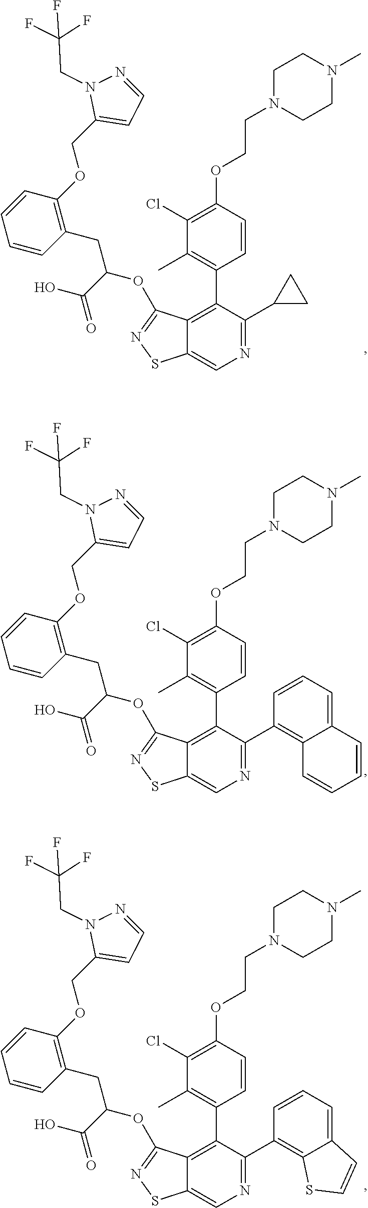 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl] 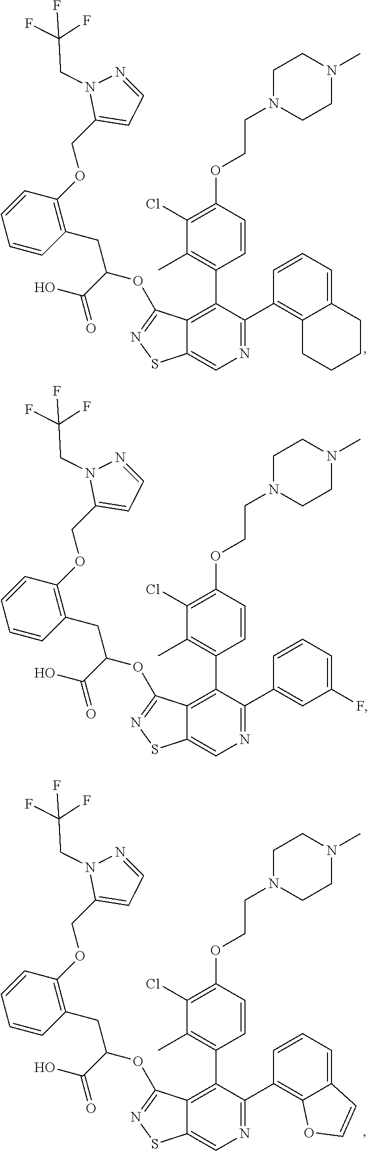 [SMILES: Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl] 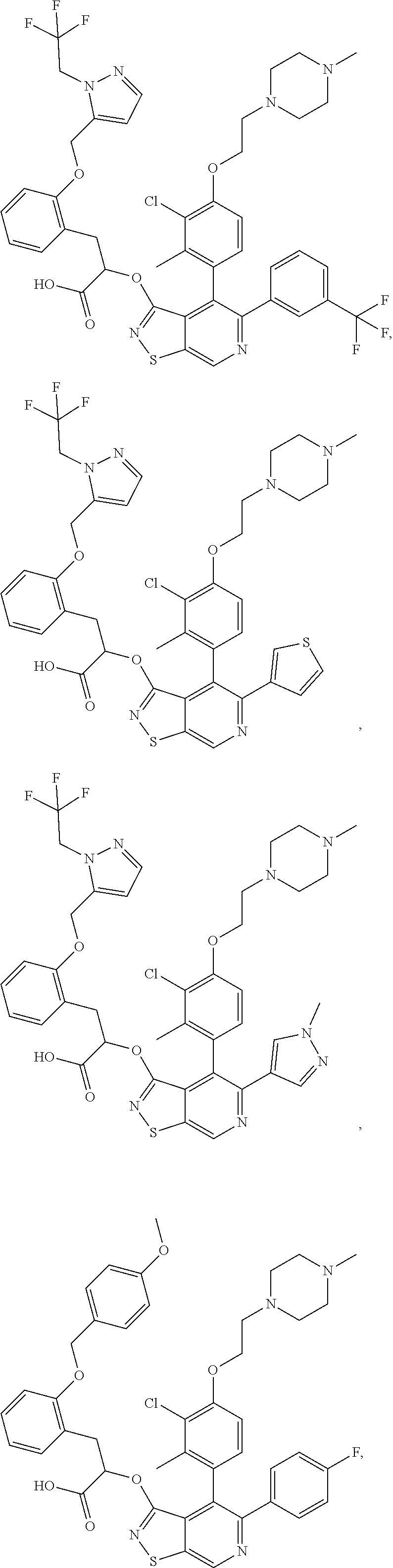 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl ; COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1] 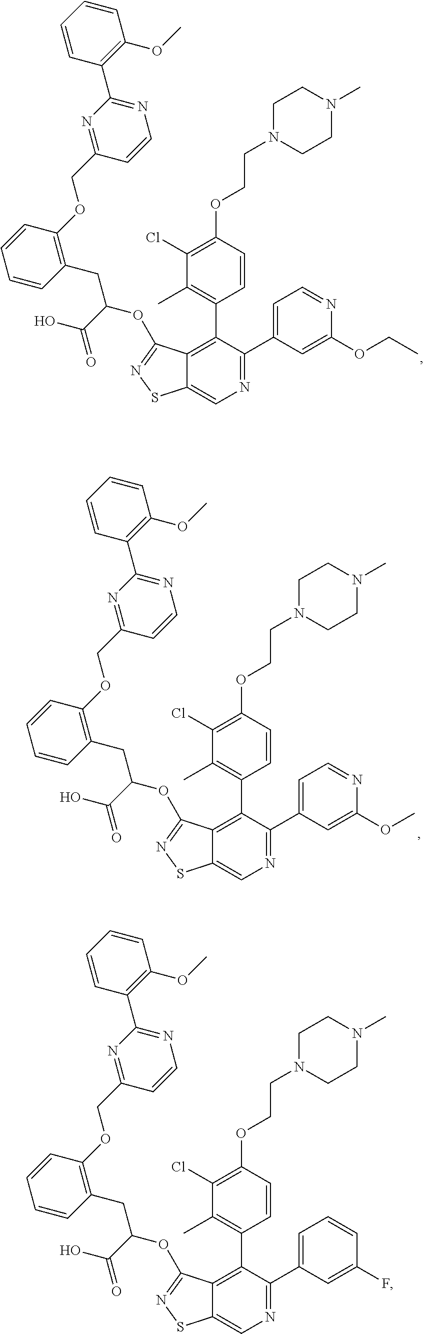 [SMILES: CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1] 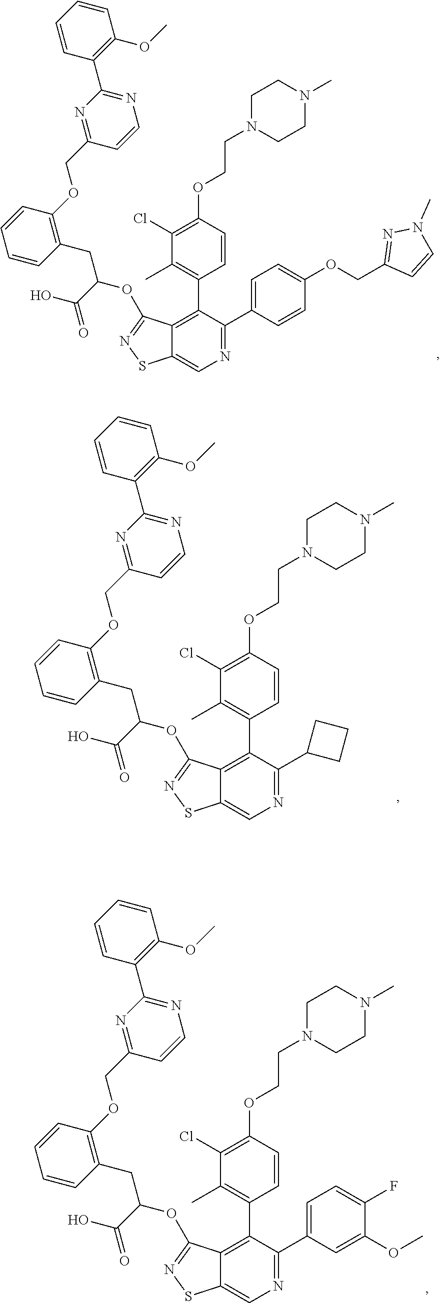 [SMILES: COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F] 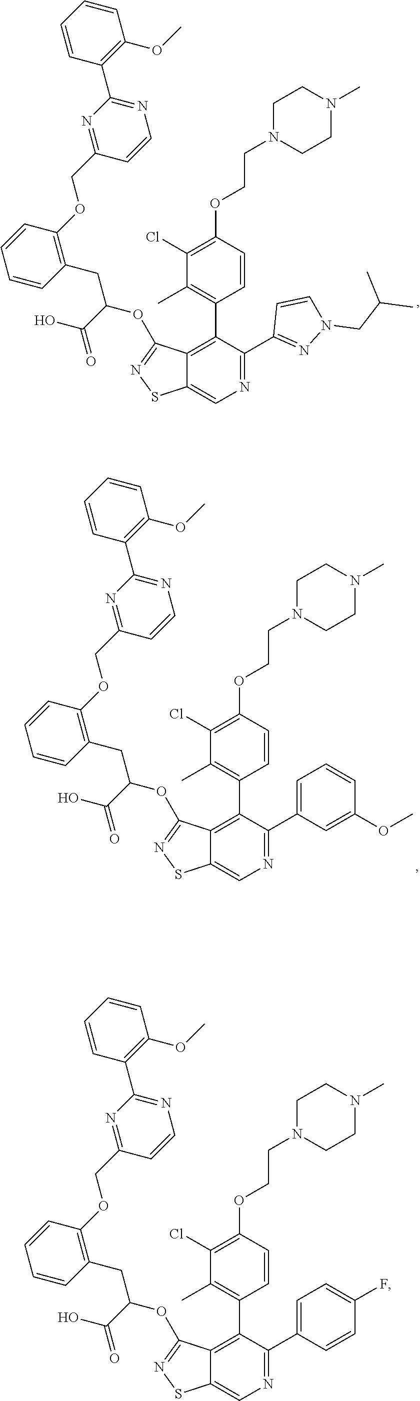 [SMILES: COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1 ; COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1] 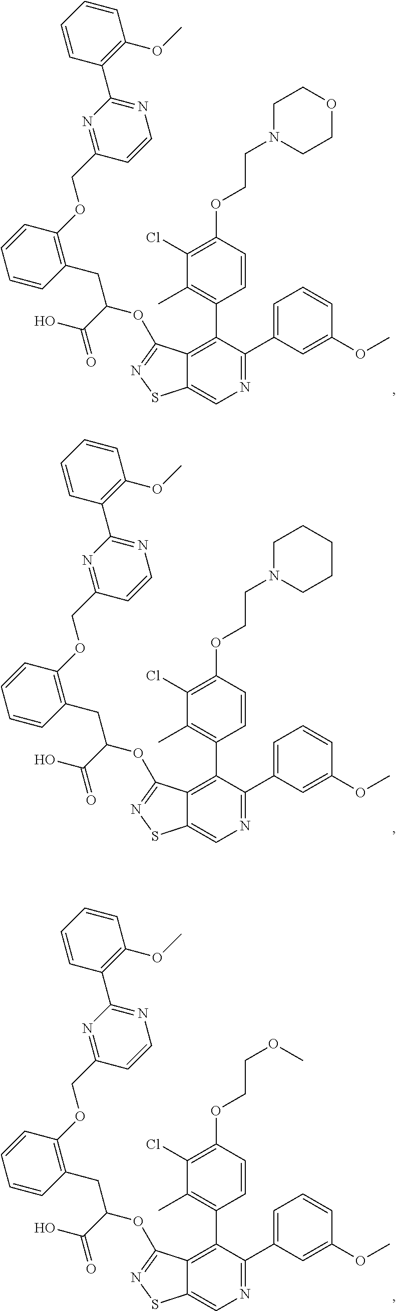 [SMILES: COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl] 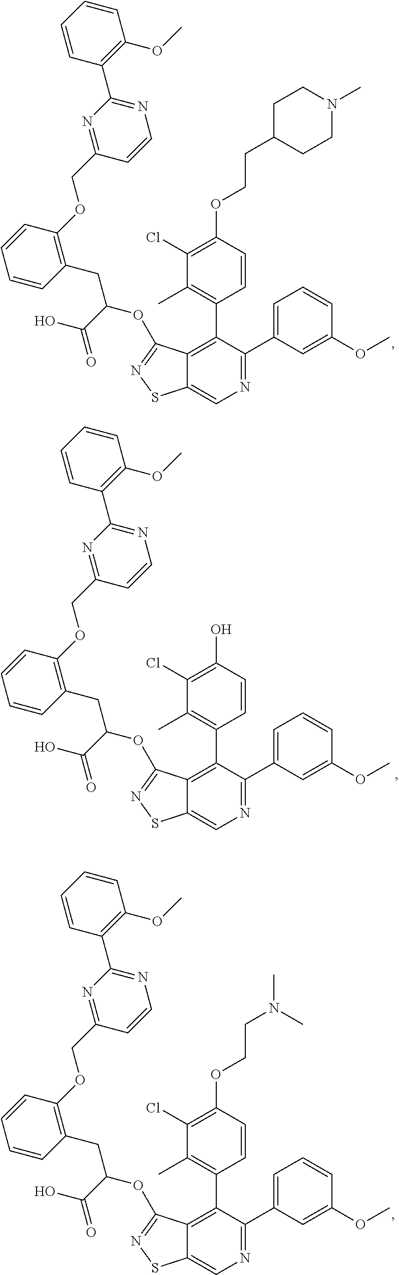 [SMILES: COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1] 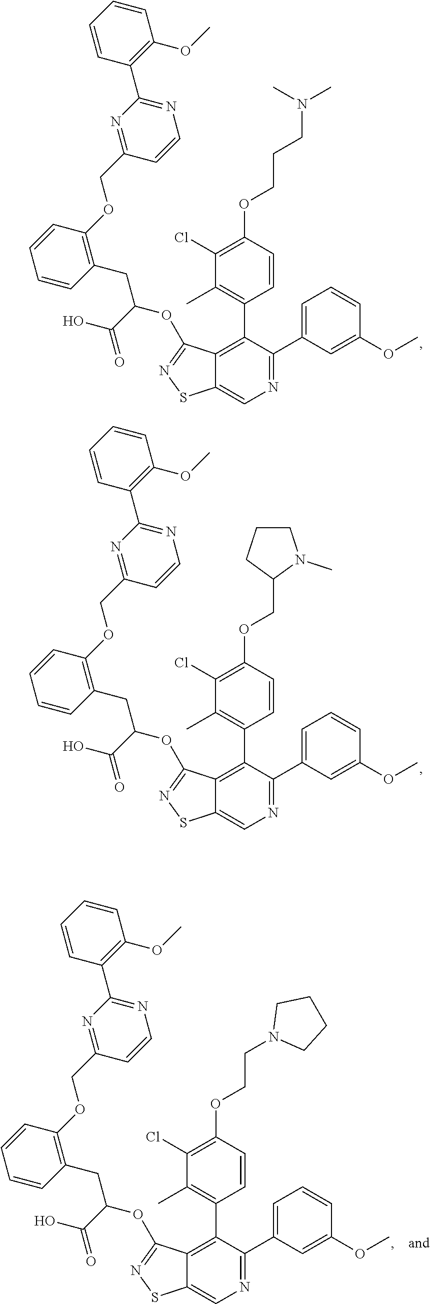 [SMILES: COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCC3CCCN3C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1] 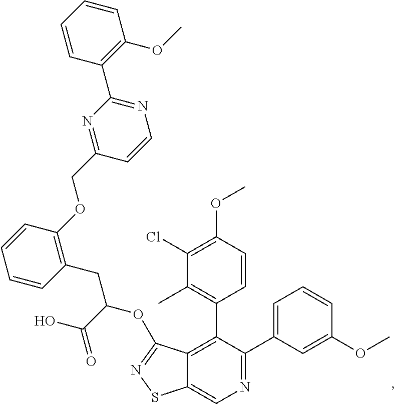 [SMILES: COc1cccc(-c2ncc3snc(OC(Cc4ccccc4OCc4ccnc(-c5ccccc5OC)n4)C(=O)O)c3c2-c2ccc(OC)c(Cl)c2C)c1]
or a pharmaceutically acceptable salt thereof.

42. A compound selected from: 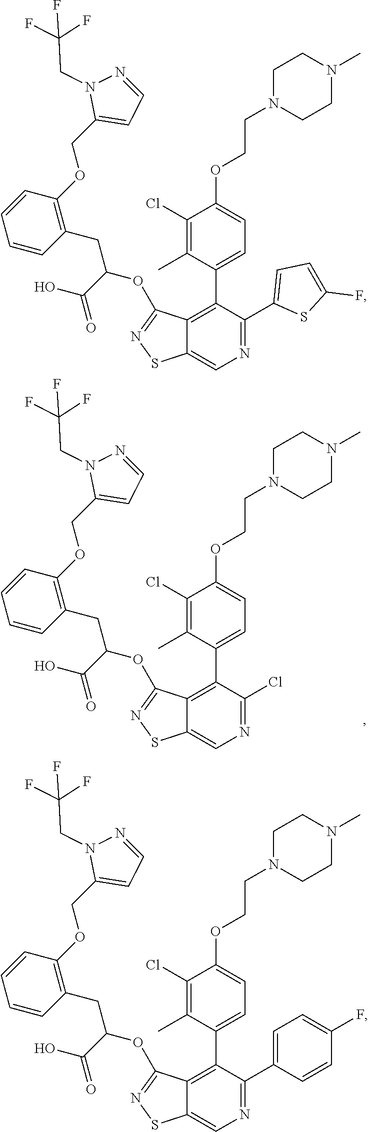 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl] 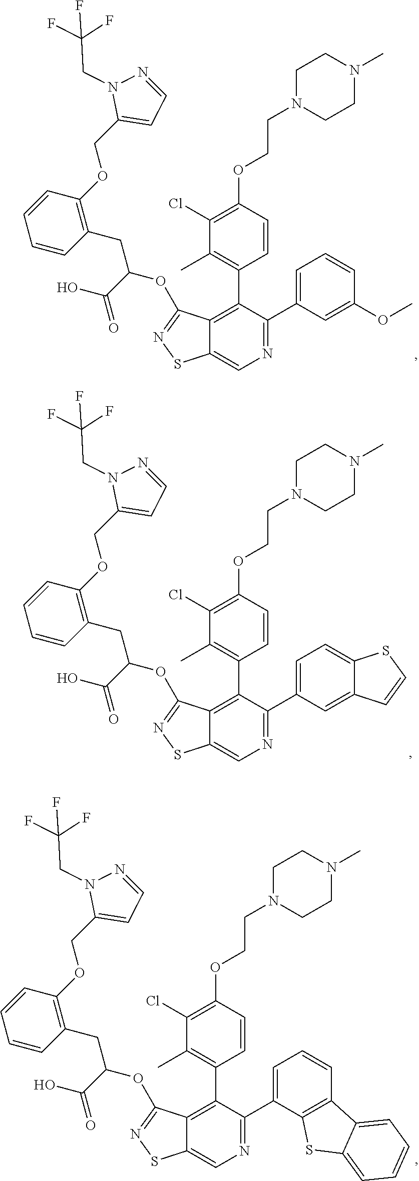 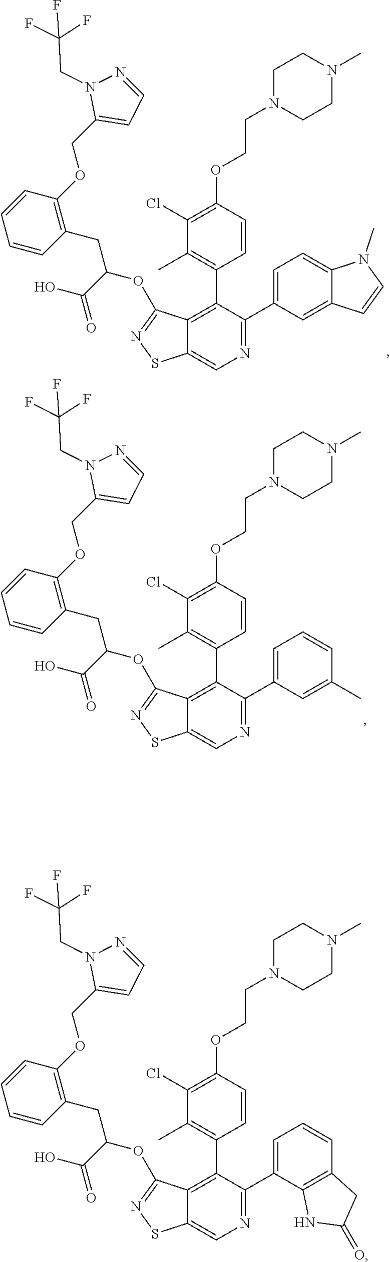 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl] 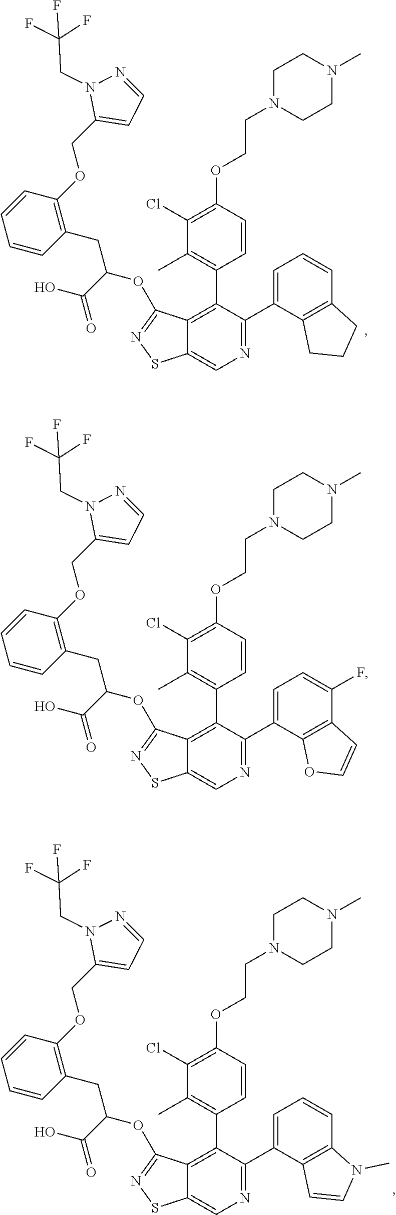 [SMILES: Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl] 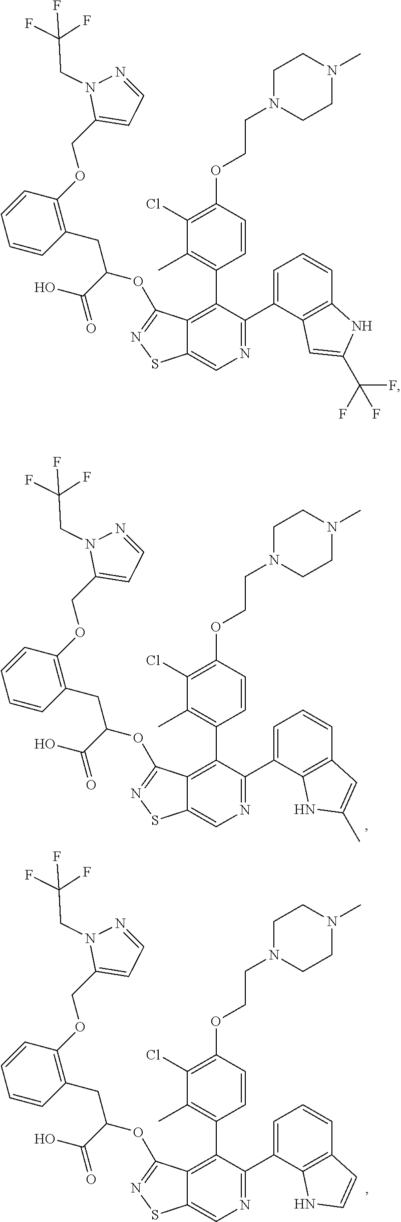 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1 ; Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl] 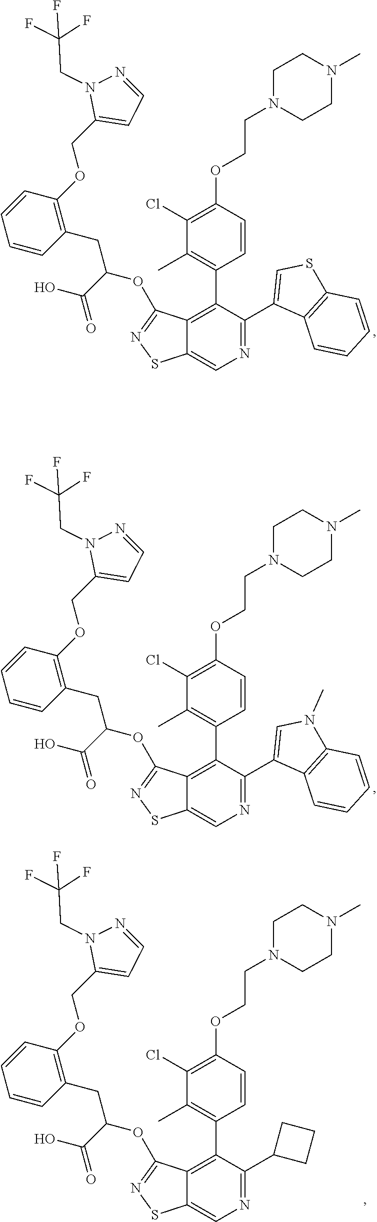 [SMILES: Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl] 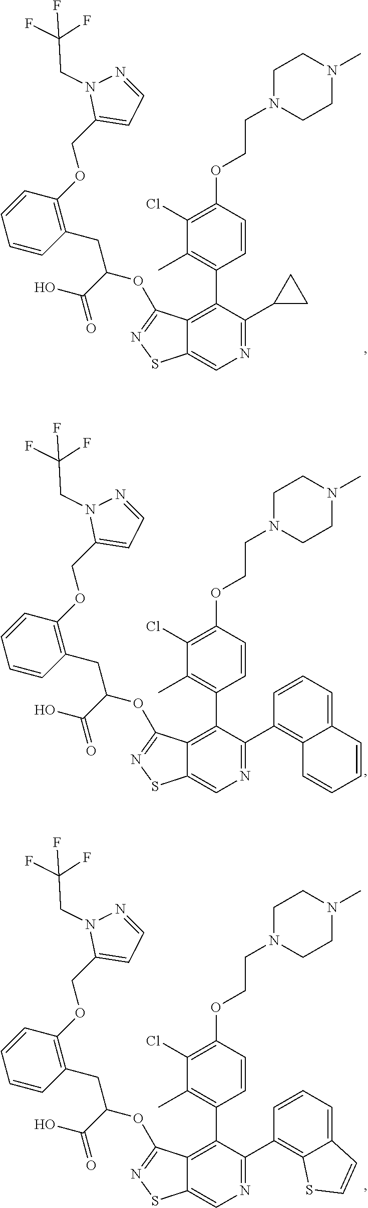 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl] 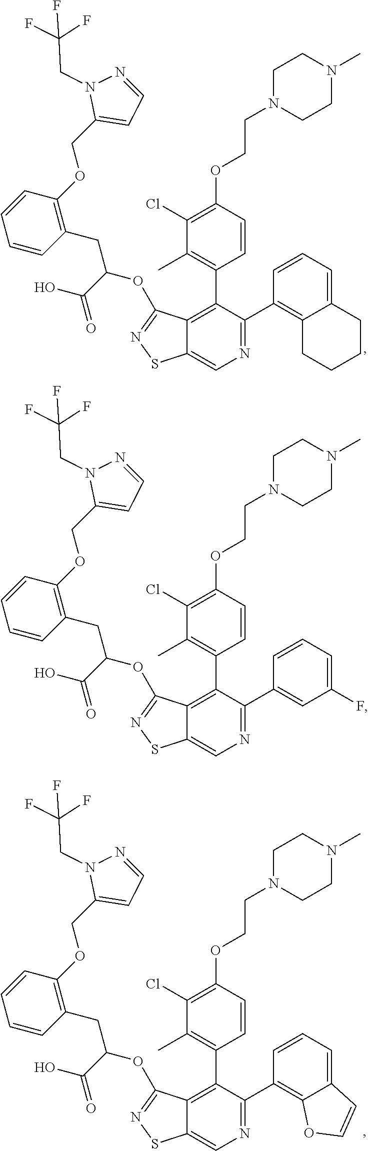 [SMILES: Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl] 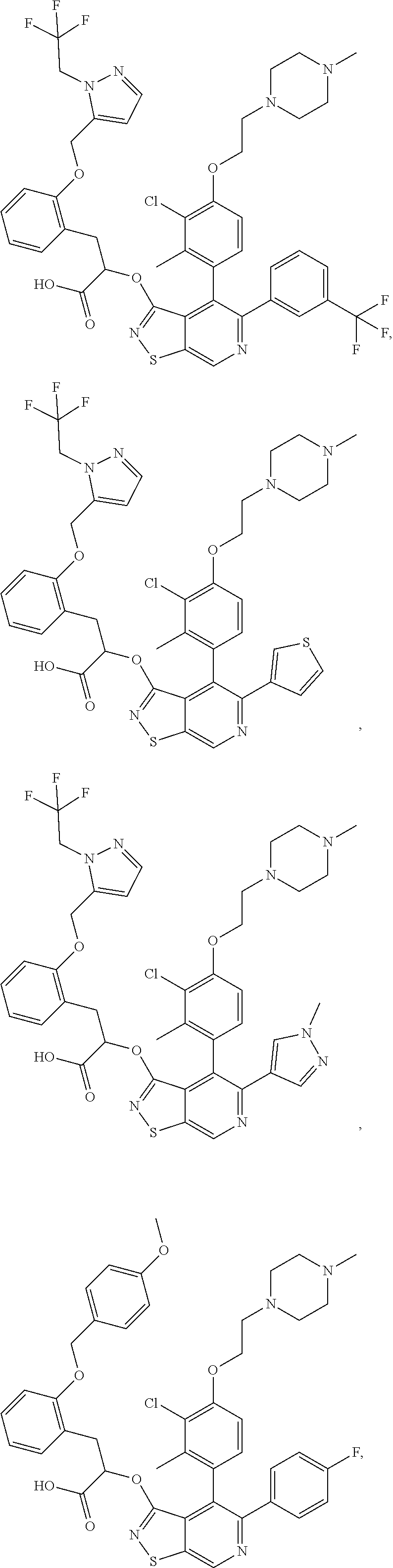 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl ; COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1] 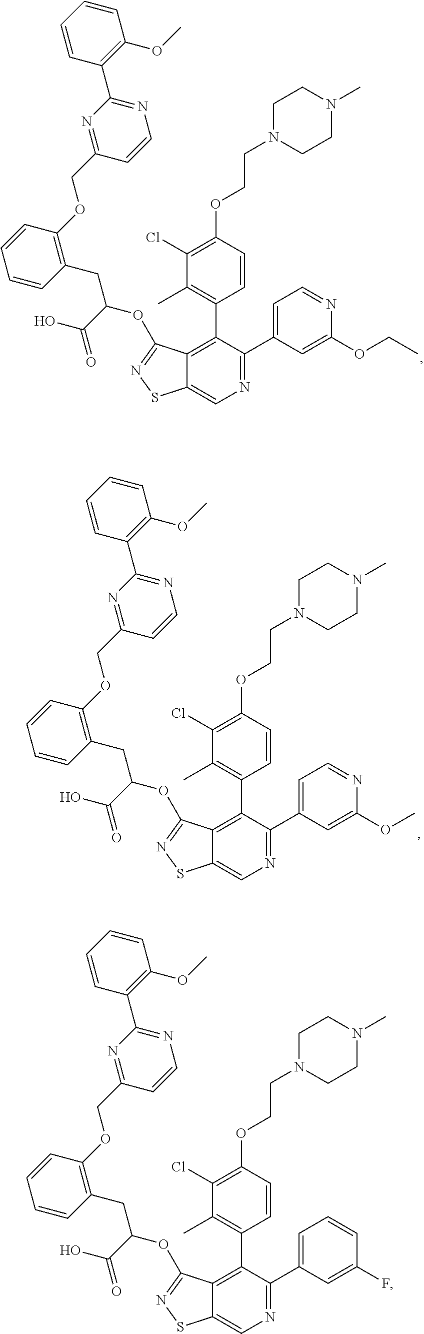 [SMILES: CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1] 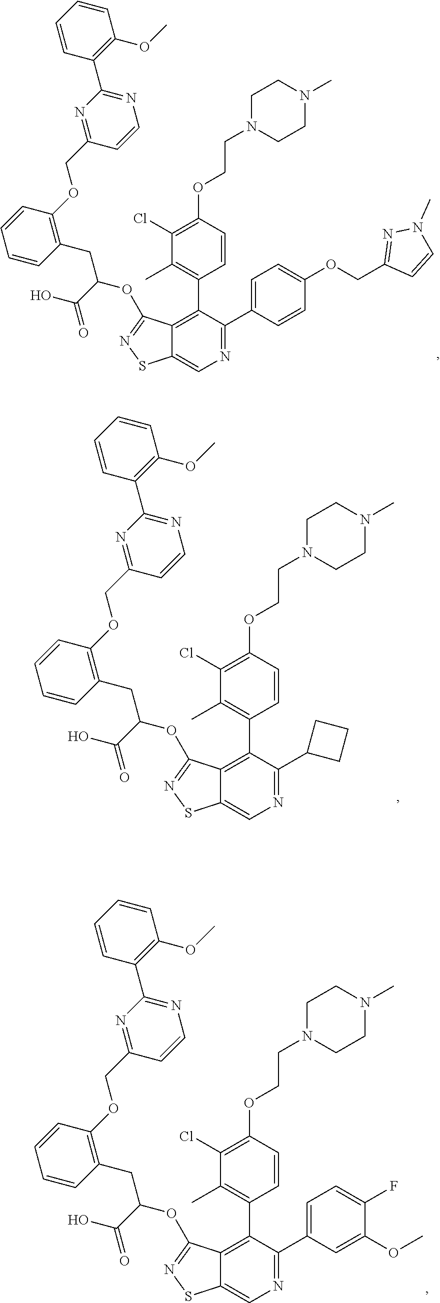 [SMILES: COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F] 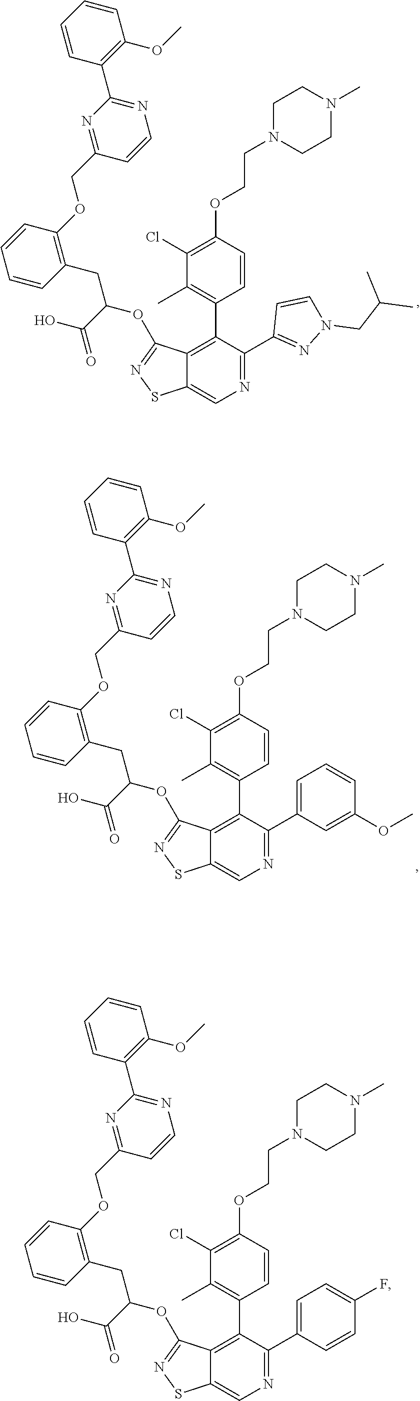 [SMILES: COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1 ; COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1] 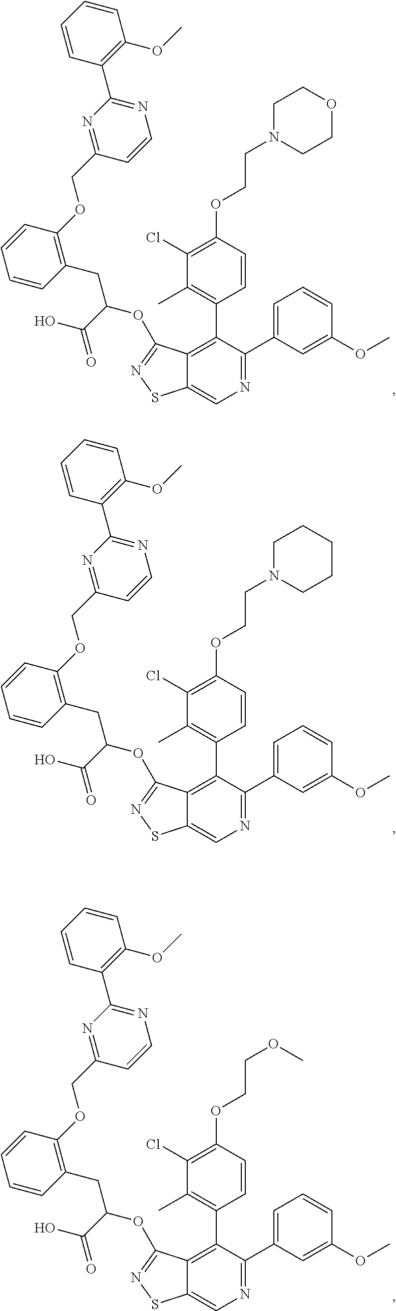 [SMILES: COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl] 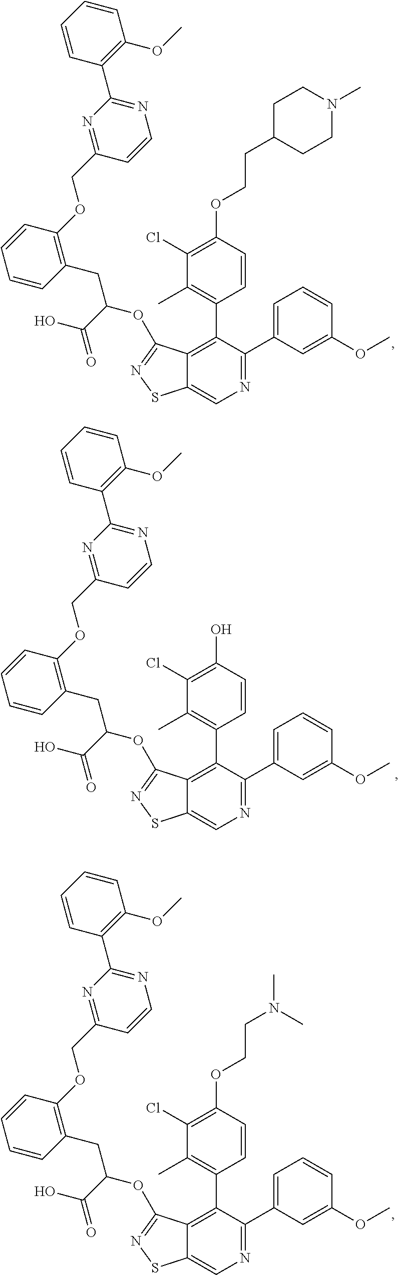 [SMILES: COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1] 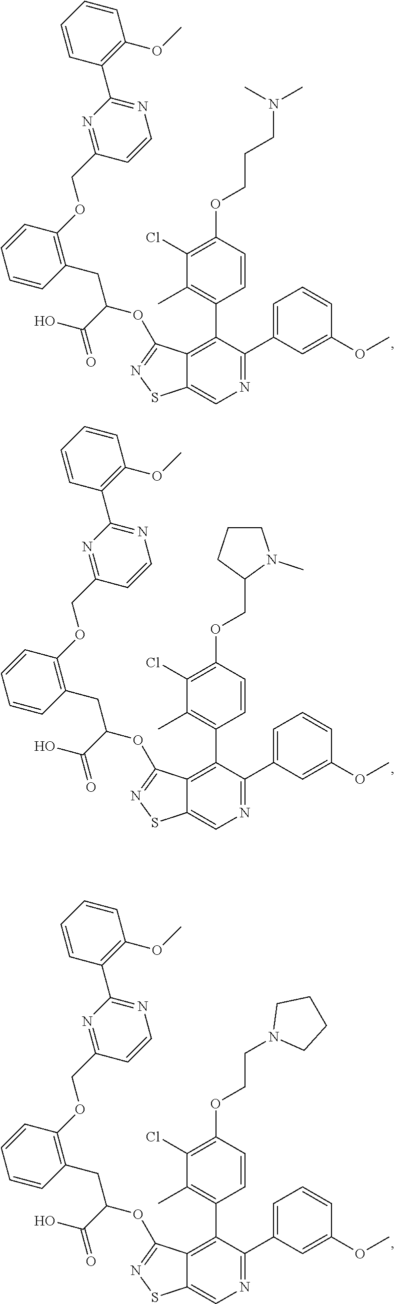 [SMILES: COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCC3CCCN3C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1] 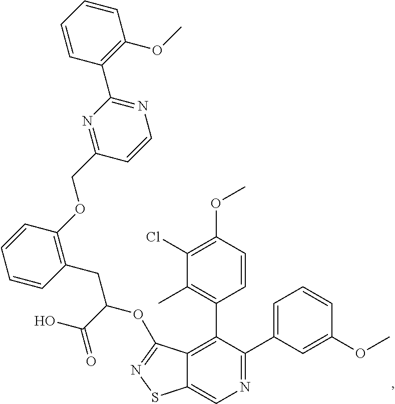 [SMILES: COc1cccc(-c2ncc3snc(OC(Cc4ccccc4OCc4ccnc(-c5ccccc5OC)n4)C(=O)O)c3c2-c2ccc(OC)c(Cl)c2C)c1] 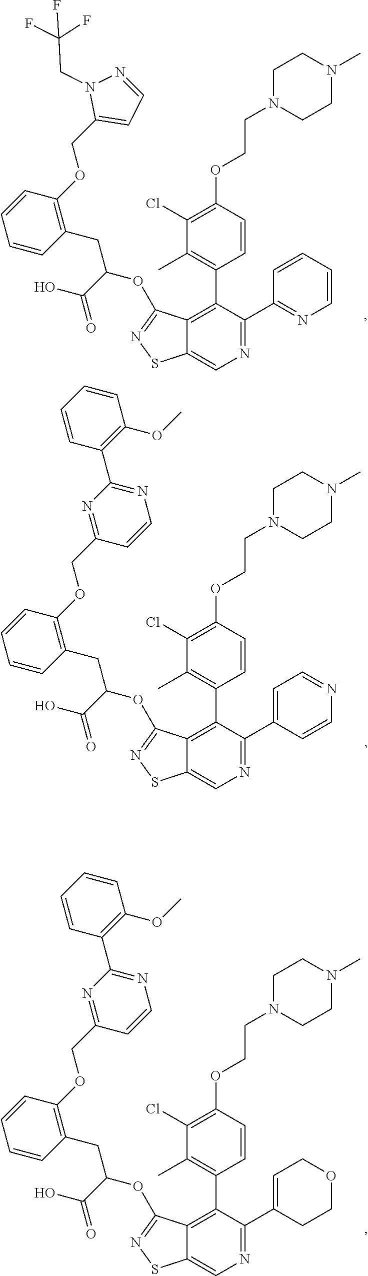 [SMILES: Cc1c(-c2c(-c3ccccn3)ncc3snc(OC(Cc4ccccc4OCc4ccnn4CC(F)(F)F)C(=O)O)c23)ccc(OCCN2CCN(C)CC2)c1Cl ; COc1ccccc1-c1nccc(COc2ccccc2CC(Oc2nsc3cnc(-c4ccncc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4=CCOCC4)c(-c4ccc(OCCN5CCN(C)CC5)c(Cl)c4C)c23)C(=O)O)n1] 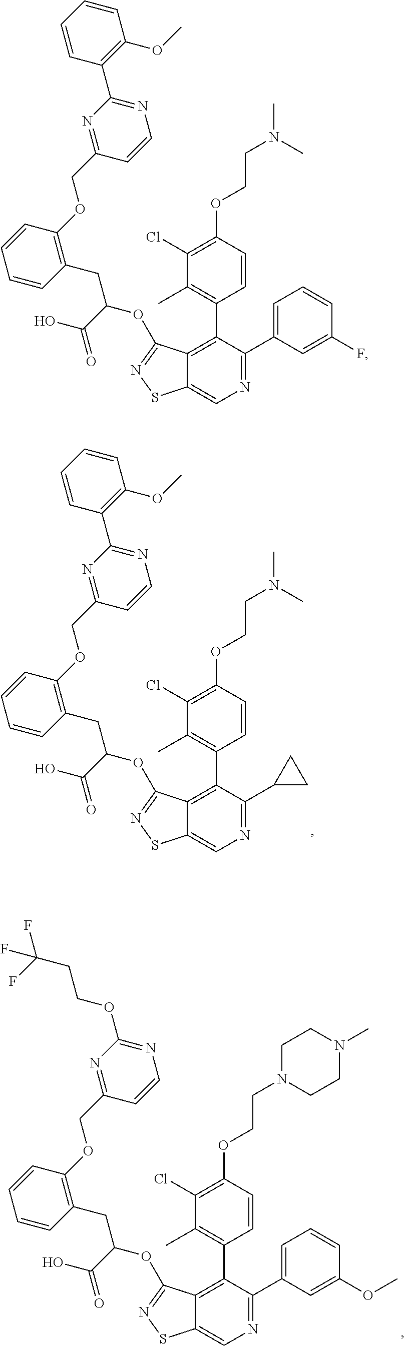 [SMILES: COc1ccccc1-c1nccc(COc2ccccc2CC(Oc2nsc3cnc(-c4cccc(F)c4)c(-c4ccc(OCCN(C)C)c(Cl)c4C)c23)C(=O)O)n1 ; COc1ccccc1-c1nccc(COc2ccccc2CC(Oc2nsc3cnc(C4CC4)c(-c4ccc(OCCN(C)C)c(Cl)c4C)c23)C(=O)O)n1 ; COc1cccc(-c2ncc3snc(OC(Cc4ccccc4OCc4ccnc(OCCC(F)(F)F)n4)C(=O)O)c3c2-c2ccc(OCCN3CCN(C)CC3)c(Cl)c2C)c1] 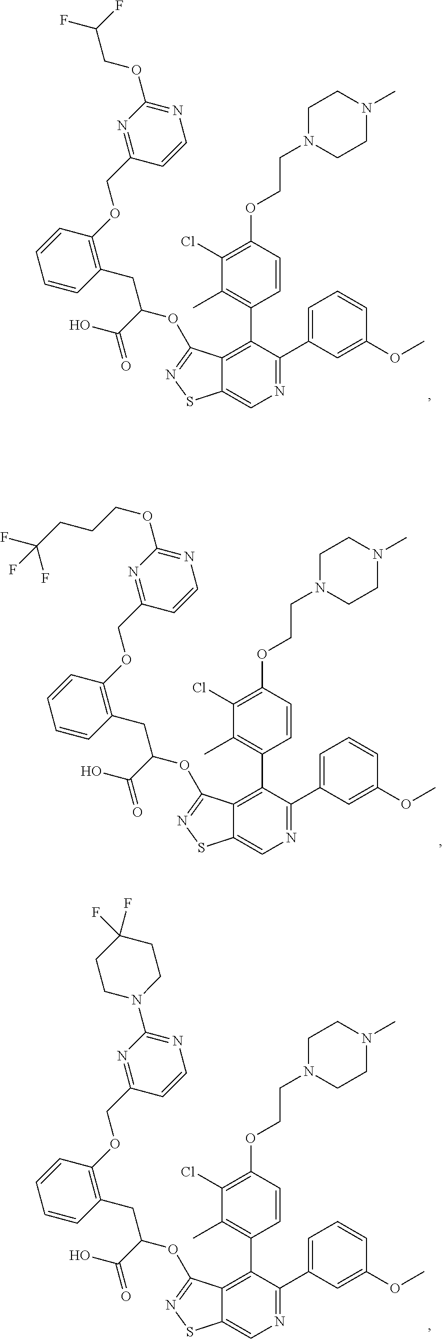 [SMILES: COc1cccc(-c2ncc3snc(OC(Cc4ccccc4OCc4ccnc(OCC(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(OCCCC(F)(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(F)(F)CC5)n4)C(=O)O)c3c2-c2ccc(OCCN3CCN(C)CC3)c(Cl)c2C)c1] 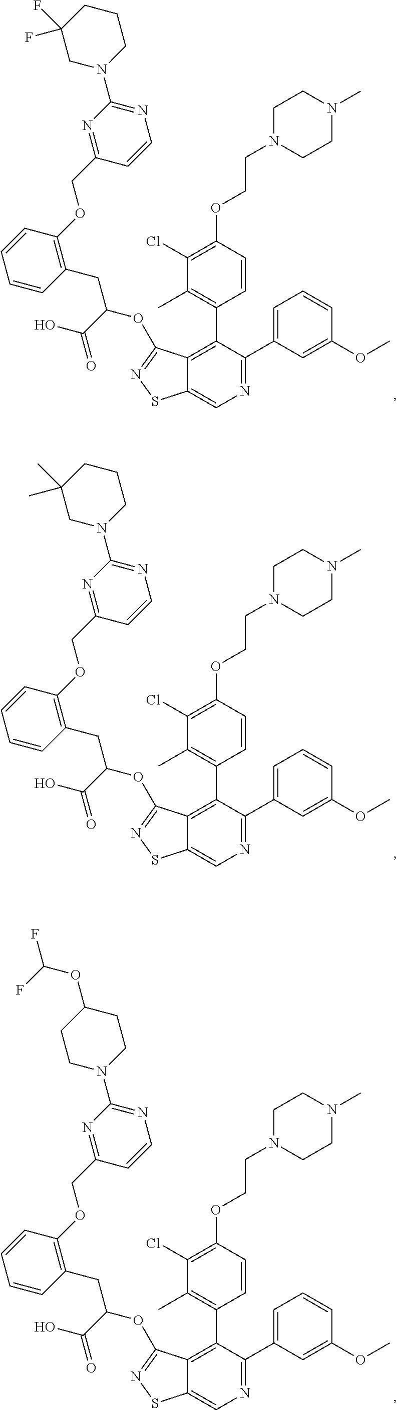 [SMILES: COc1cccc(-c2ncc3snc(OC(Cc4ccccc4OCc4ccnc(N5CCCC(F)(F)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCCC(C)(C)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(OC(F)F)CC5)n4)C(=O)O)c3c2-c2ccc(OCCN3CCN(C)CC3)c(Cl)c2C)c1] 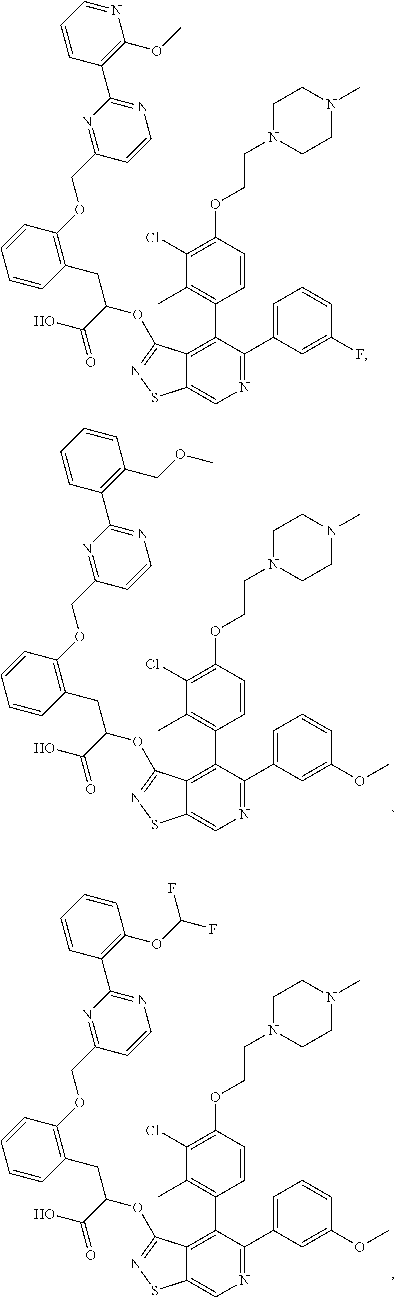 [SMILES: COc1ncccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1 ; COCc1ccccc1-c1nccc(COc2ccccc2CC(Oc2nsc3cnc(-c4cccc(OC)c4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC(F)F)n4)C(=O)O)c3c2-c2ccc(OCCN3CCN(C)CC3)c(Cl)c2C)c1] 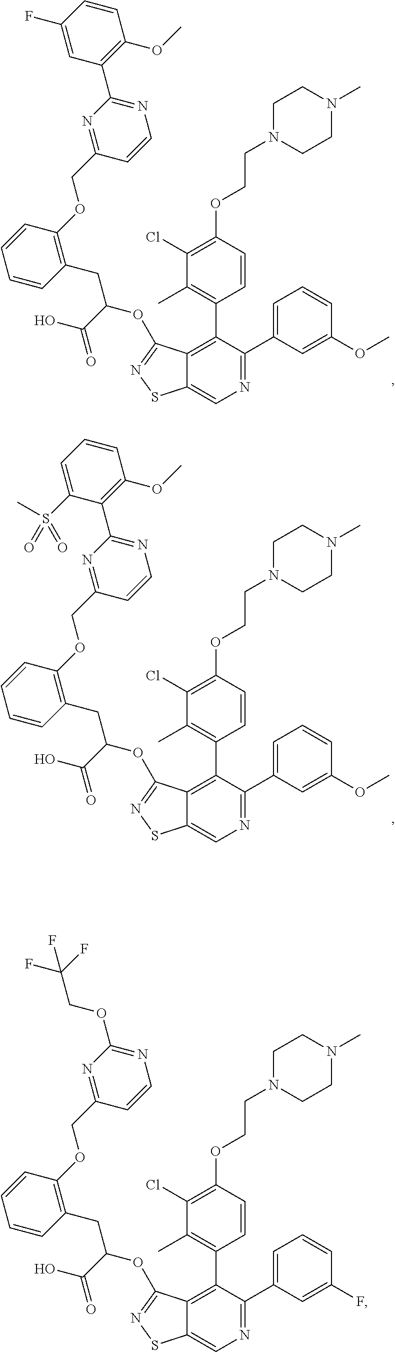 [SMILES: COc1cccc(-c2ncc3snc(OC(Cc4ccccc4OCc4ccnc(-c5cc(F)ccc5OC)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(-c5c(OC)cccc5S(C)(=O)=O)n4)C(=O)O)c3c2-c2ccc(OCCN3CCN(C)CC3)c(Cl)c2C)c1 ; Cc1c(-c2c(-c3cccc(F)c3)ncc3snc(OC(Cc4ccccc4OCc4ccnc(OCC(F)(F)F)n4)C(=O)O)c23)ccc(OCCN2CCN(C)CC2)c1Cl] 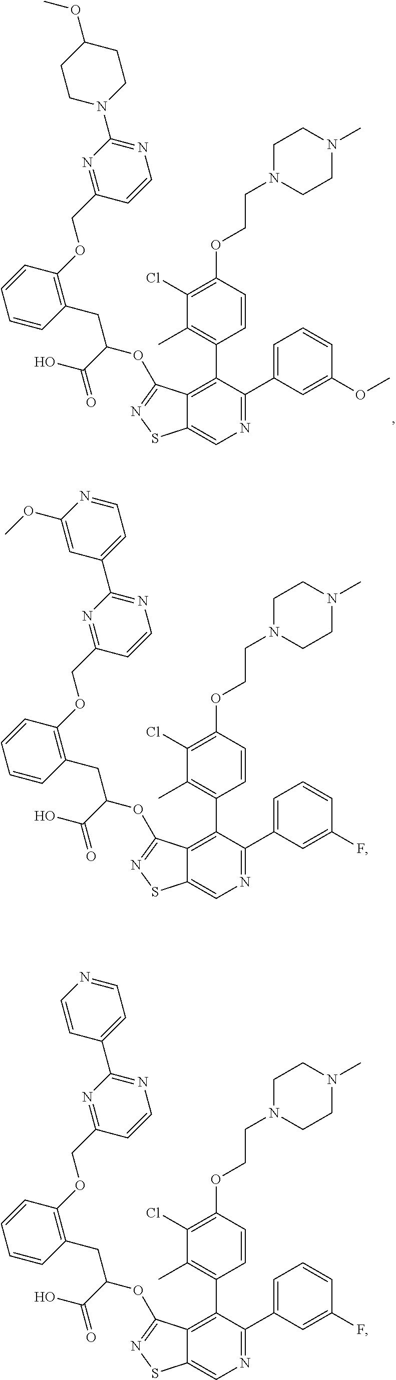 [SMILES: COc1cccc(-c2ncc3snc(OC(Cc4ccccc4OCc4ccnc(N5CCC(OC)CC5)n4)C(=O)O)c3c2-c2ccc(OCCN3CCN(C)CC3)c(Cl)c2C)c1 ; COc1cc(-c2nccc(COc3ccccc3CC(Oc3nsc4cnc(-c5cccc(F)c5)c(-c5ccc(OCCN6CCN(C)CC6)c(Cl)c5C)c34)C(=O)O)n2)ccn1 ; Cc1c(-c2c(-c3cccc(F)c3)ncc3snc(OC(Cc4ccccc4OCc4ccnc(-c5ccncc5)n4)C(=O)O)c23)ccc(OCCN2CCN(C)CC2)c1Cl] 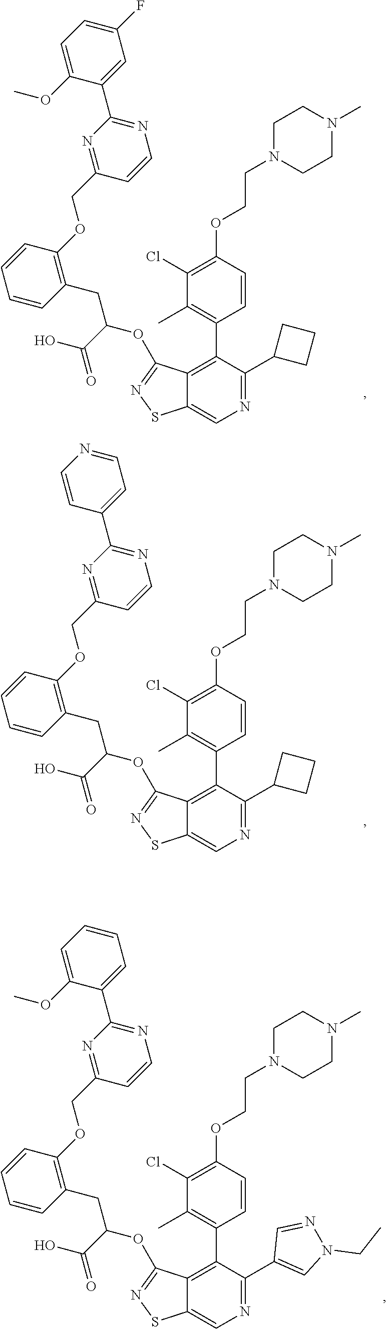 [SMILES: COc1ccc(F)cc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5ccncc5)n4)C(=O)O)c23)ccc(OCCN2CCN(C)CC2)c1Cl ; CCn1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)cn1] 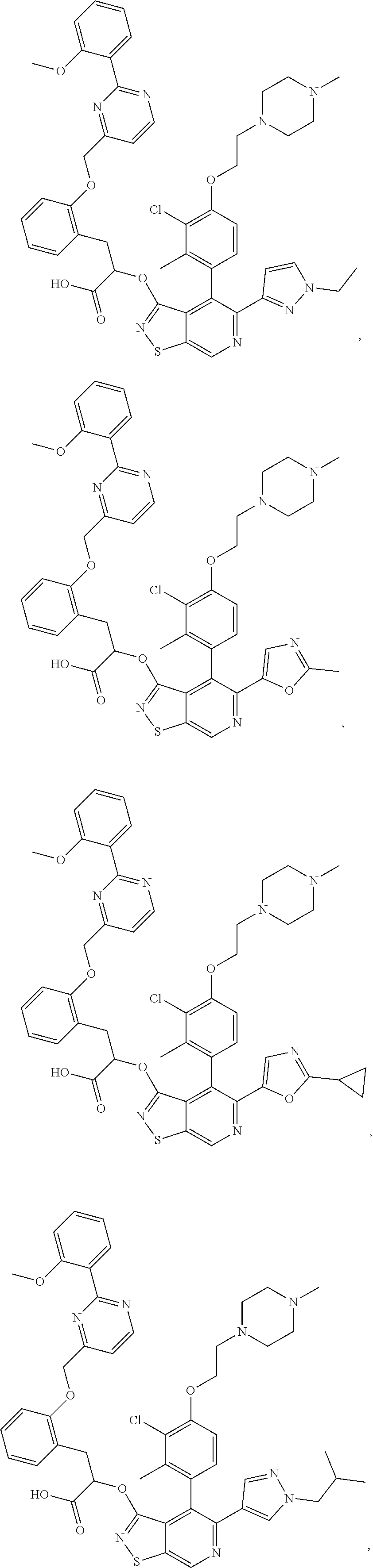 [SMILES: CCn1ccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)n1 ; COc1ccccc1-c1nccc(COc2ccccc2CC(Oc2nsc3cnc(-c4cnc(C)o4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnc(C5CC5)o4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC(C)C)c4)c(-c4ccc(OCCN5CCN(C)CC5)c(Cl)c4C)c23)C(=O)O)n1] 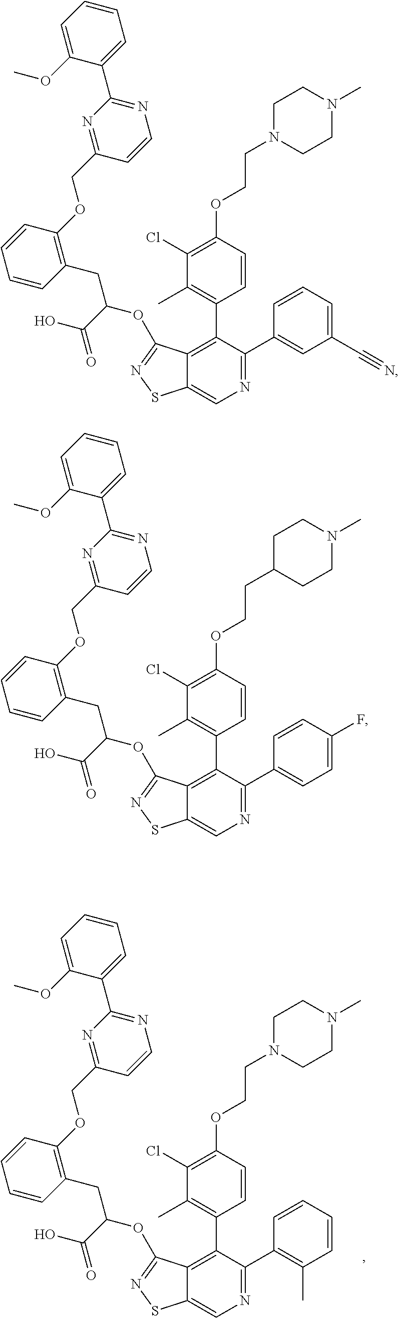 [SMILES: COc1ccccc1-c1nccc(COc2ccccc2CC(Oc2nsc3cnc(-c4cccc(C#N)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(F)cc4)c(-c4ccc(OCCC5CCN(C)CC5)c(Cl)c4C)c23)C(=O)O)n1 ; COc1ccccc1-c1nccc(COc2ccccc2CC(Oc2nsc3cnc(-c4ccccc4C)c(-c4ccc(OCCN5CCN(C)CC5)c(Cl)c4C)c23)C(=O)O)n1] 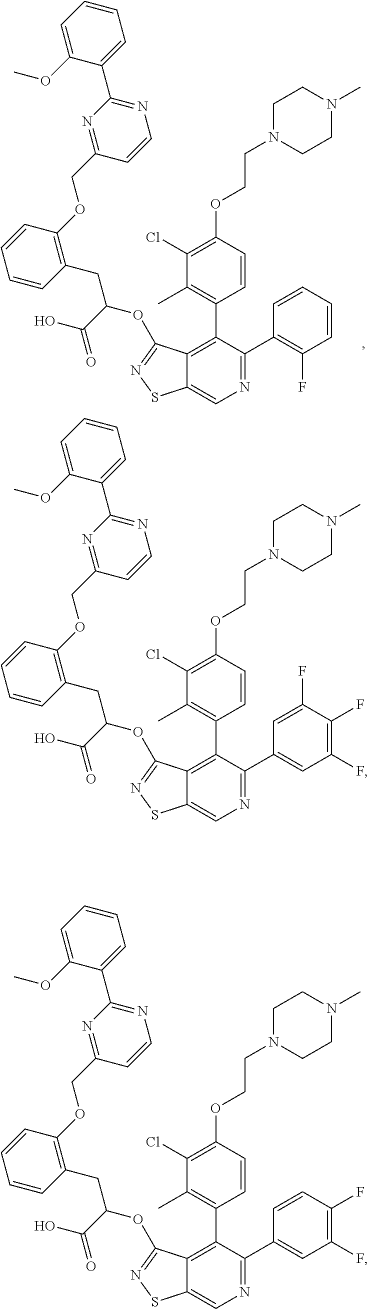 [SMILES: COc1ccccc1-c1nccc(COc2ccccc2CC(Oc2nsc3cnc(-c4ccccc4F)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cc(F)c(F)c(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(F)c(F)c4)c(-c4ccc(OCCN5CCN(C)CC5)c(Cl)c4C)c23)C(=O)O)n1] 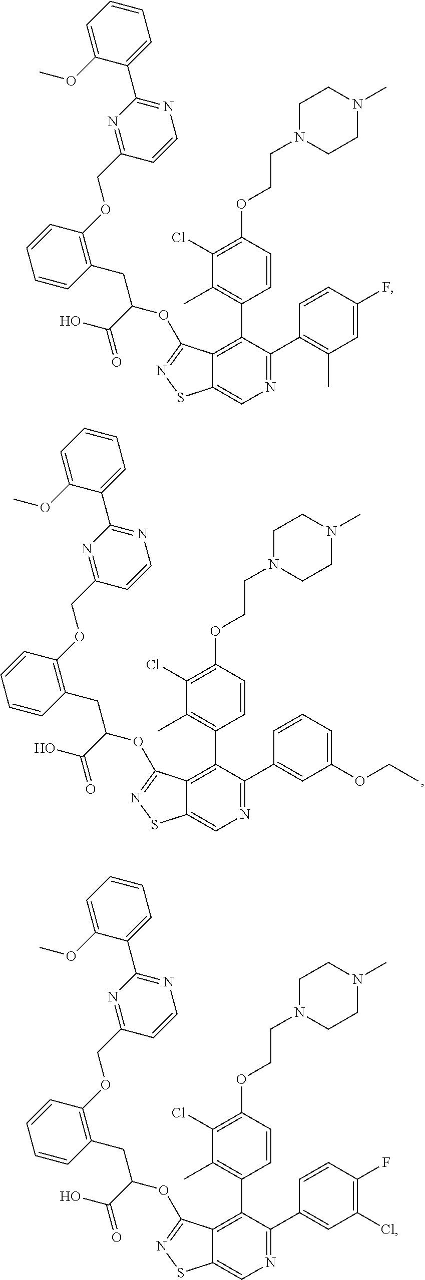 [SMILES: COc1ccccc1-c1nccc(COc2ccccc2CC(Oc2nsc3cnc(-c4ccc(F)cc4C)c(-c4ccc(OCCN5CCN(C)CC5)c(Cl)c4C)c23)C(=O)O)n1 ; CCOc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1 ; COc1ccccc1-c1nccc(COc2ccccc2CC(Oc2nsc3cnc(-c4ccc(F)c(Cl)c4)c(-c4ccc(OCCN5CCN(C)CC5)c(Cl)c4C)c23)C(=O)O)n1] 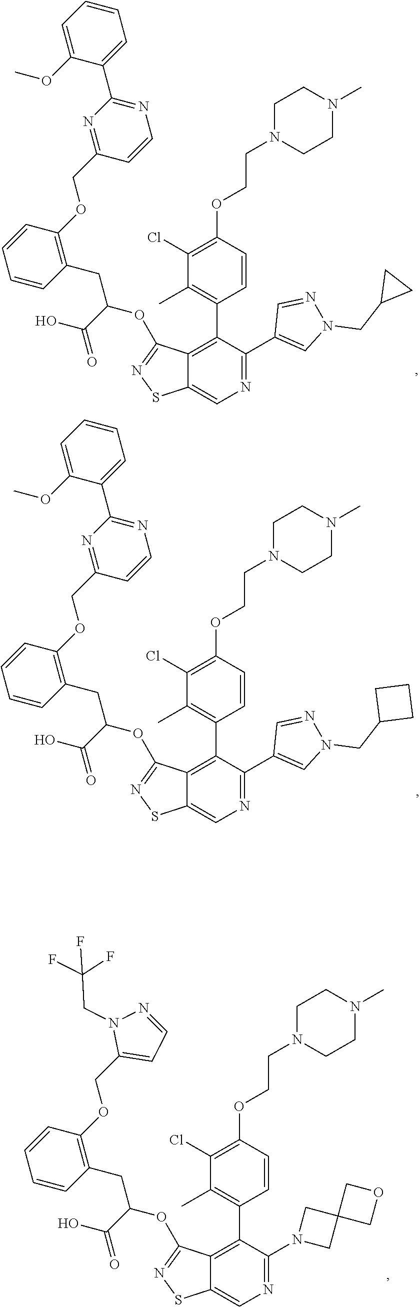 [SMILES: COc1ccccc1-c1nccc(COc2ccccc2CC(Oc2nsc3cnc(-c4cnn(CC5CC5)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CCC5)c4)c(-c4ccc(OCCN5CCN(C)CC5)c(Cl)c4C)c23)C(=O)O)n1 ; Cc1c(-c2c(N3CC4(COC4)C3)ncc3snc(OC(Cc4ccccc4OCc4ccnn4CC(F)(F)F)C(=O)O)c23)ccc(OCCN2CCN(C)CC2)c1Cl] 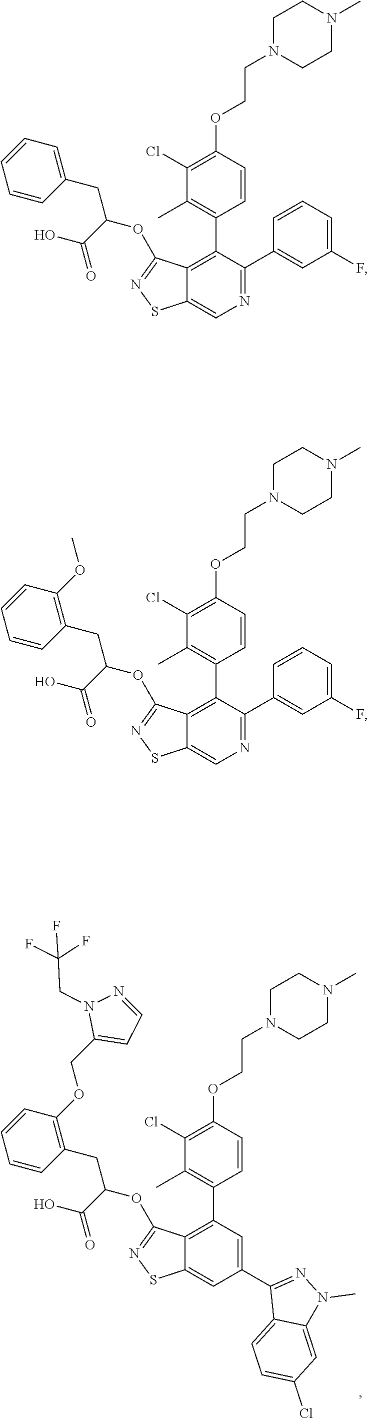 [SMILES: Cc1c(-c2c(-c3cccc(F)c3)ncc3snc(OC(Cc4ccccc4)C(=O)O)c23)ccc(OCCN2CCN(C)CC2)c1Cl ; COc1ccccc1CC(Oc1nsc2cnc(-c3cccc(F)c3)c(-c3ccc(OCCN4CCN(C)CC4)c(Cl)c3C)c12)C(=O)O ; Cc1c(-c2cc(-c3nn(C)c4cc(Cl)ccc34)cc3snc(OC(Cc4ccccc4OCc4ccnn4CC(F)(F)F)C(=O)O)c23)ccc(OCCN2CCN(C)CC2)c1Cl] 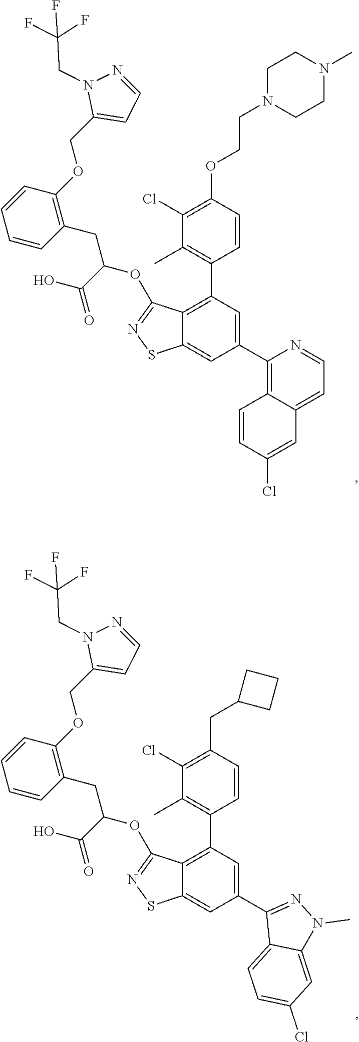 [SMILES: Cc1c(-c2cc(-c3nccc4cc(Cl)ccc34)cc3snc(OC(Cc4ccccc4OCc4ccnn4CC(F)(F)F)C(=O)O)c23)ccc(OCCN2CCN(C)CC2)c1Cl ; Cc1c(-c2cc(-c3nn(C)c4cc(Cl)ccc34)cc3snc(OC(Cc4ccccc4OCc4ccnn4CC(F)(F)F)C(=O)O)c23)ccc(CC2CCC2)c1Cl] 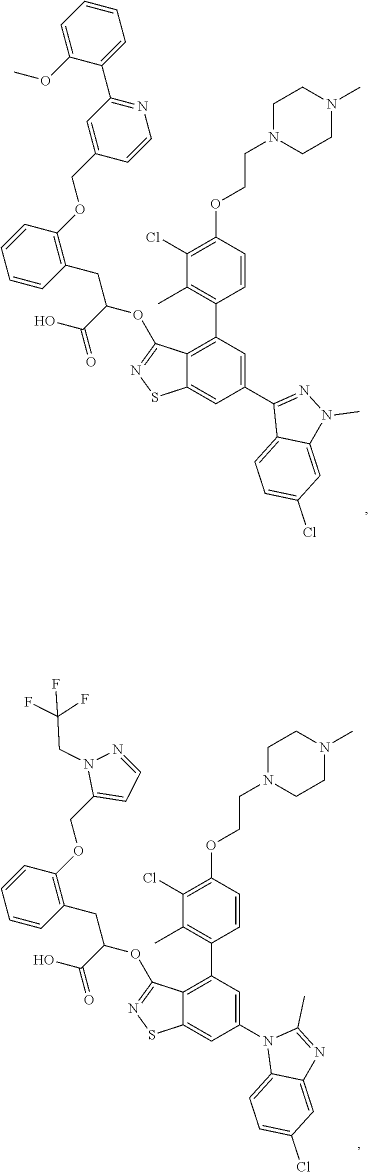 [SMILES: COc1ccccc1-c1cc(COc2ccccc2CC(Oc2nsc3cc(-c4nn(C)c5cc(Cl)ccc45)cc(-c4ccc(OCCN5CCN(C)CC5)c(Cl)c4C)c23)C(=O)O)ccn1 ; Cc1c(-c2cc(-n3c(C)nc4cc(Cl)ccc43)cc3snc(OC(Cc4ccccc4OCc4ccnn4CC(F)(F)F)C(=O)O)c23)ccc(OCCN2CCN(C)CC2)c1Cl] 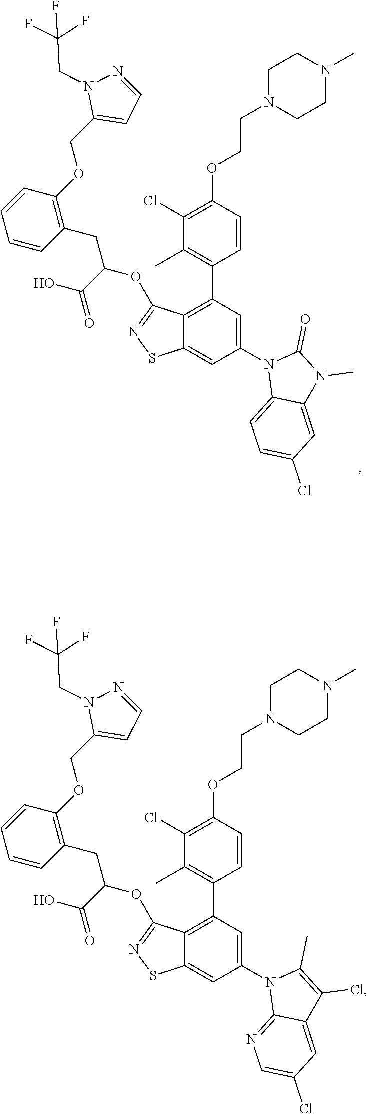 [SMILES: Cc1c(-c2cc(-n3c(=O)n(C)c4cc(Cl)ccc43)cc3snc(OC(Cc4ccccc4OCc4ccnn4CC(F)(F)F)C(=O)O)c23)ccc(OCCN2CCN(C)CC2)c1Cl ; Cc1c(-c2cc(-n3c(C)c(Cl)c4cc(Cl)cnc43)cc3snc(OC(Cc4ccccc4OCc4ccnn4CC(F)(F)F)C(=O)O)c23)ccc(OCCN2CCN(C)CC2)c1Cl] 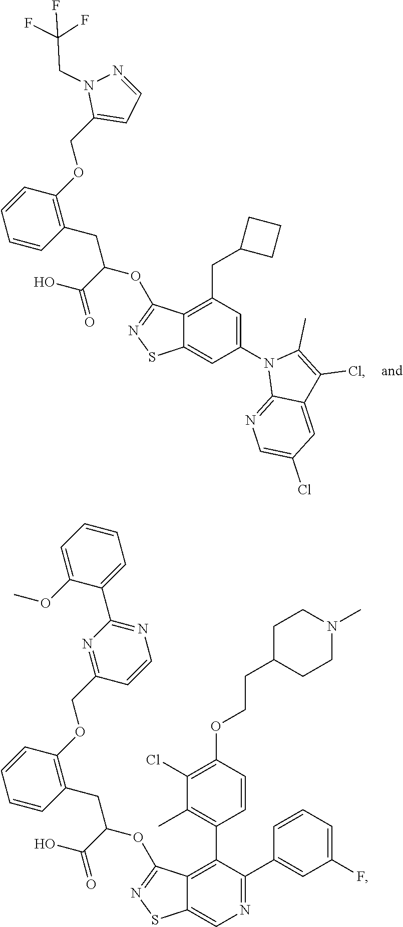 [SMILES: Cc1c(Cl)c2cc(Cl)cnc2n1-c1cc(CC2CCC2)c2c(OC(Cc3ccccc3OCc3ccnn3CC(F)(F)F)C(=O)O)nsc2c1 ; COc1ccccc1-c1nccc(COc2ccccc2CC(Oc2nsc3cnc(-c4cccc(F)c4)c(-c4ccc(OCCC5CCN(C)CC5)c(Cl)c4C)c23)C(=O)O)n1]
or a pharmaceutically acceptable salt thereof.

43. A compound selected from: 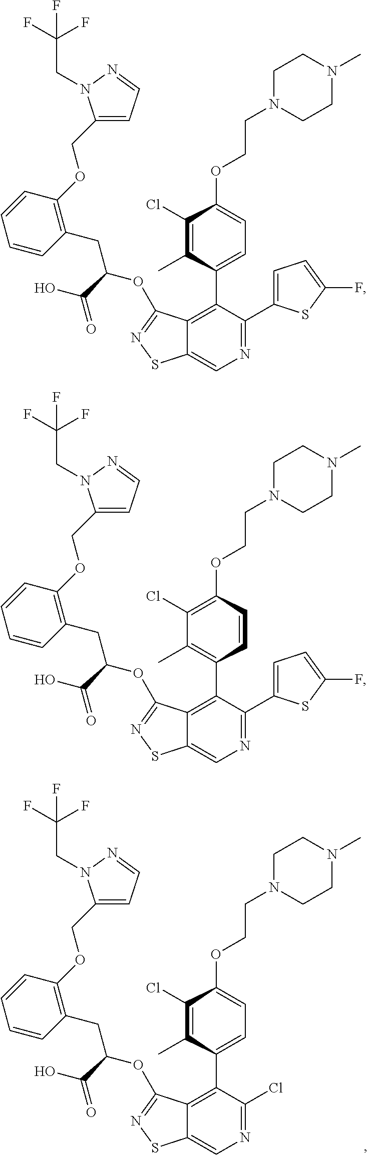 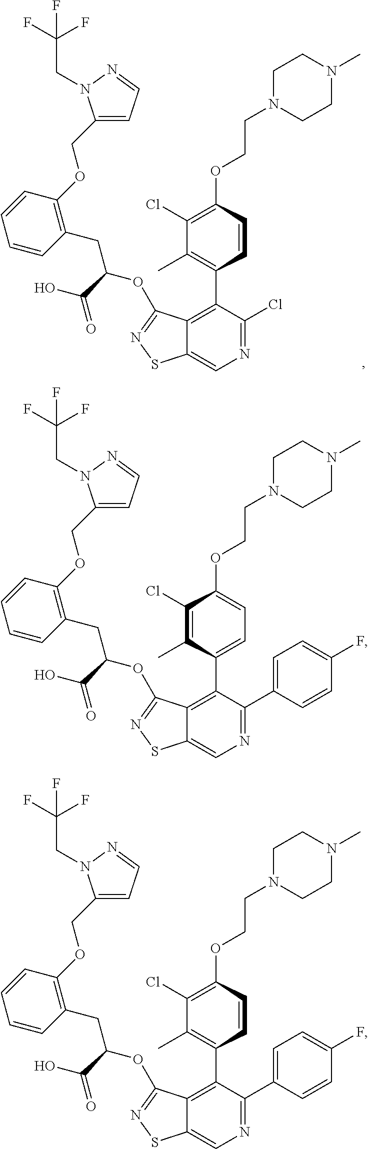 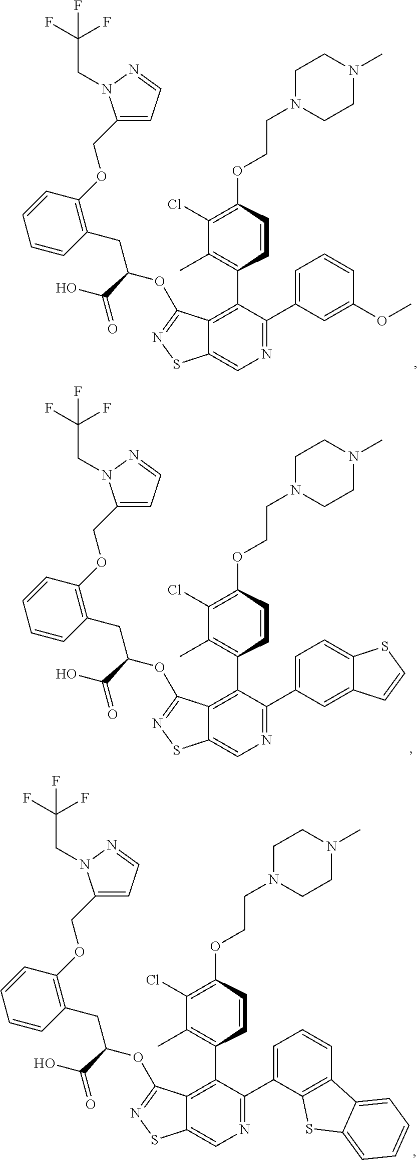 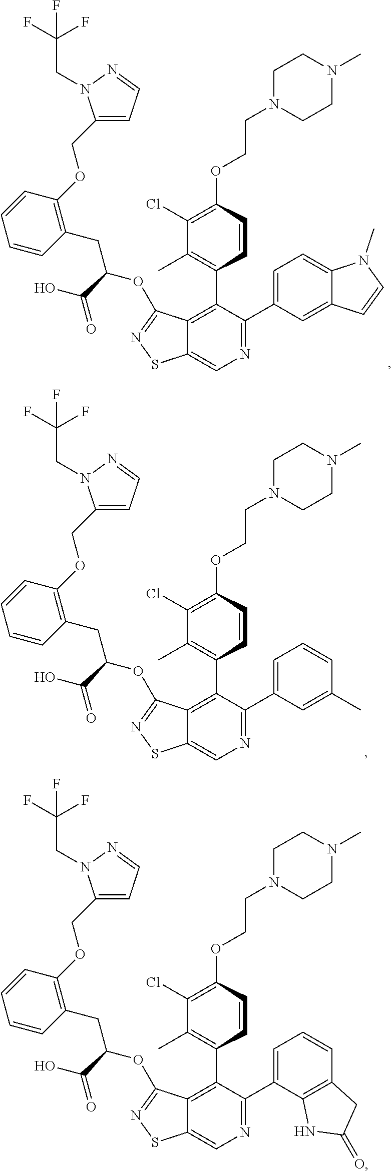 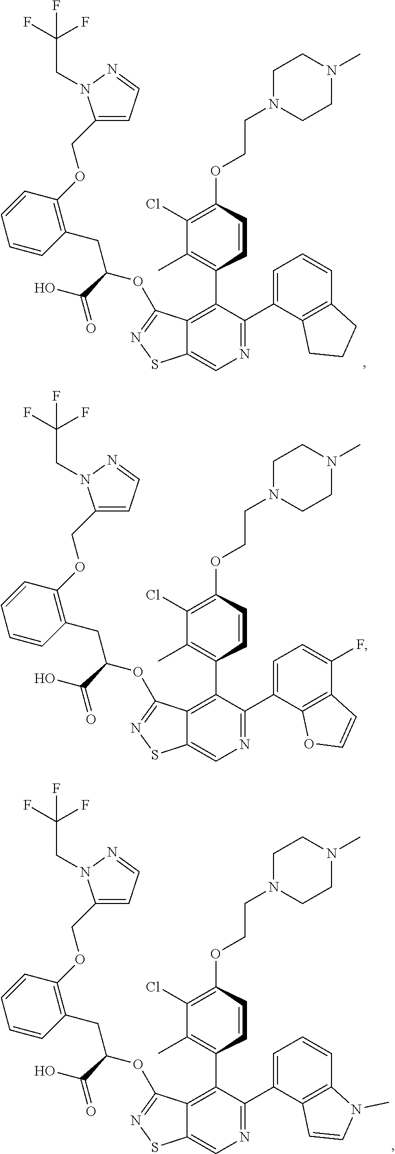 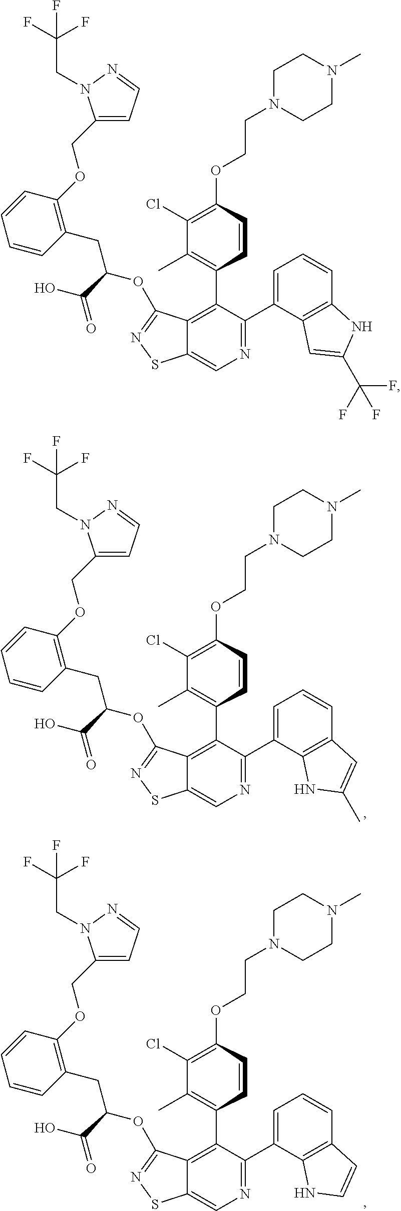 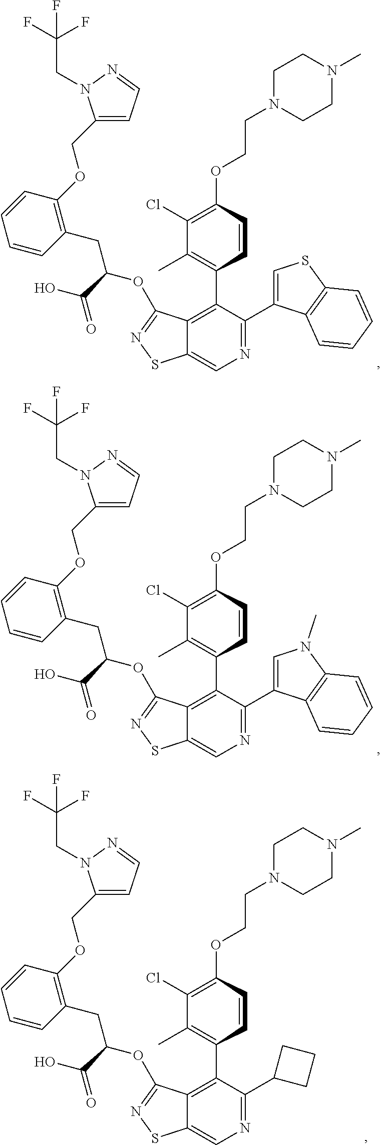 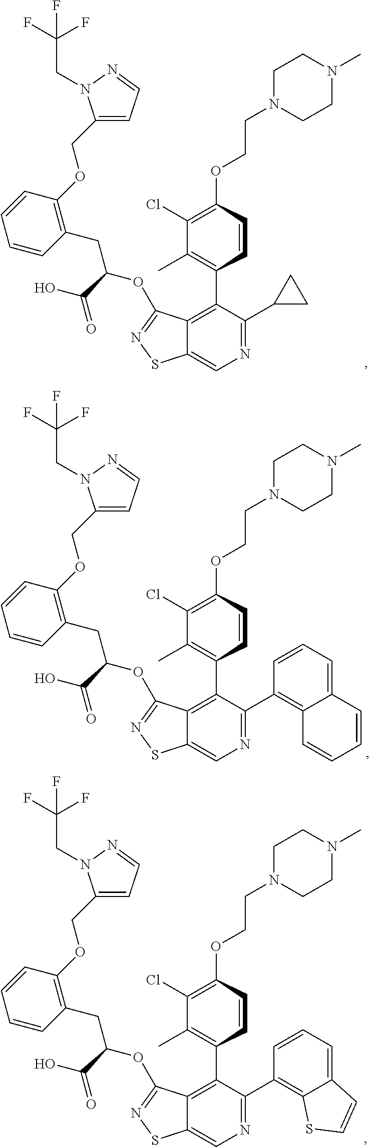 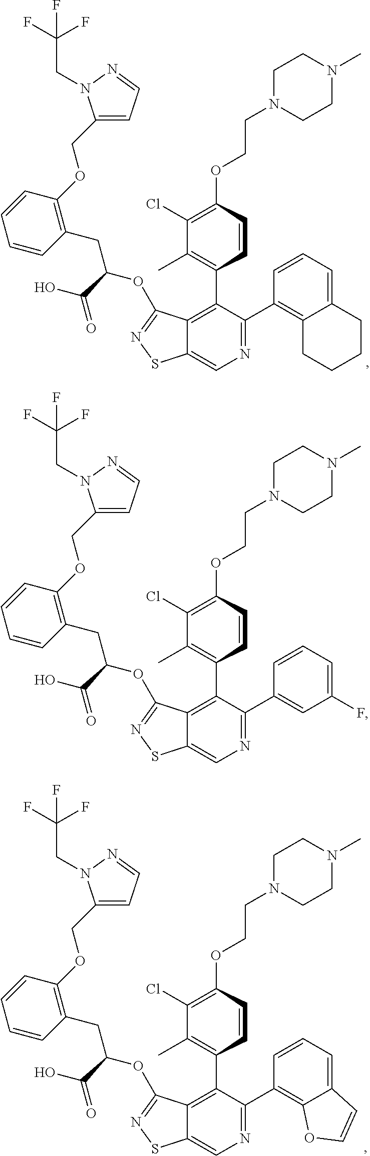 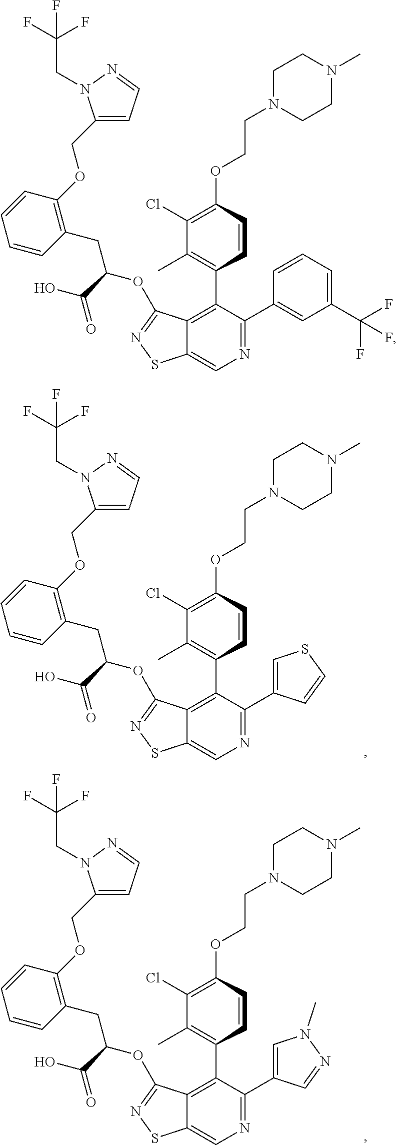 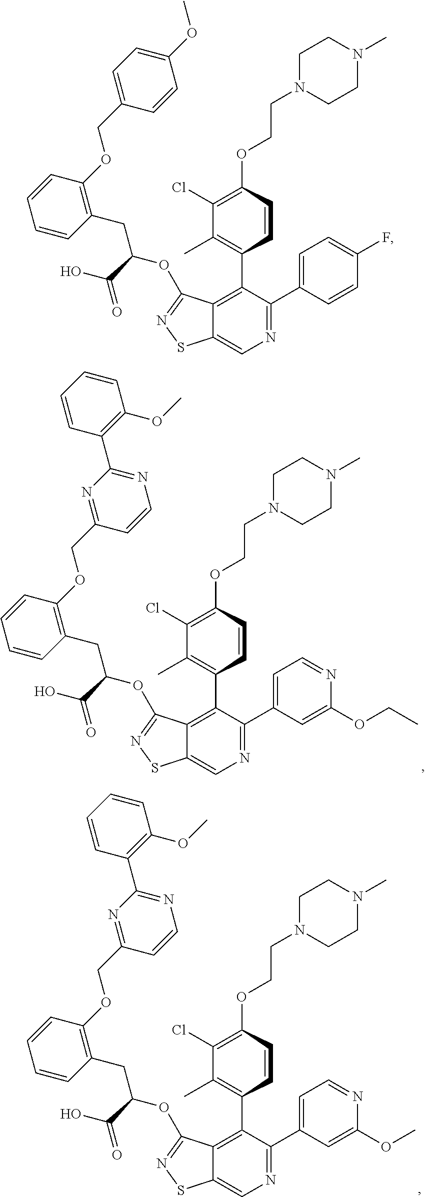 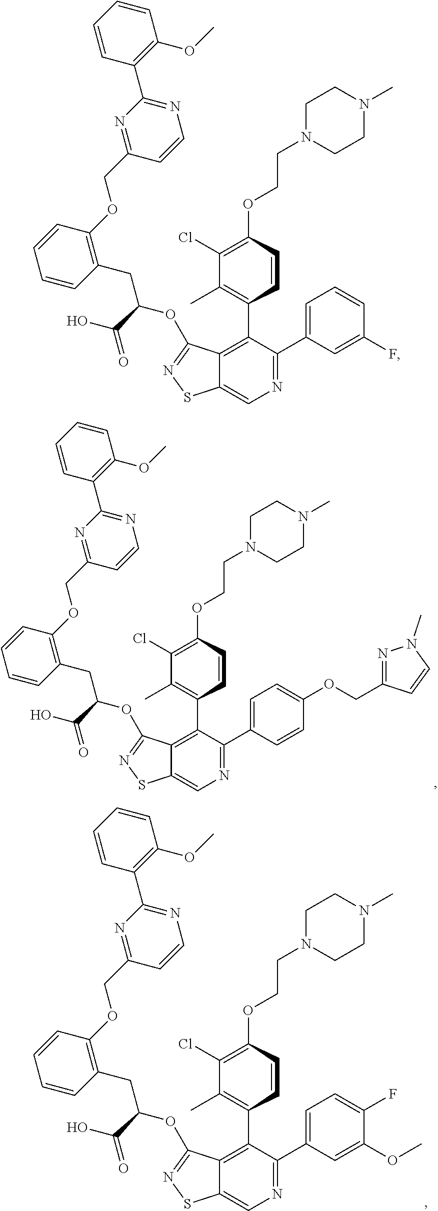 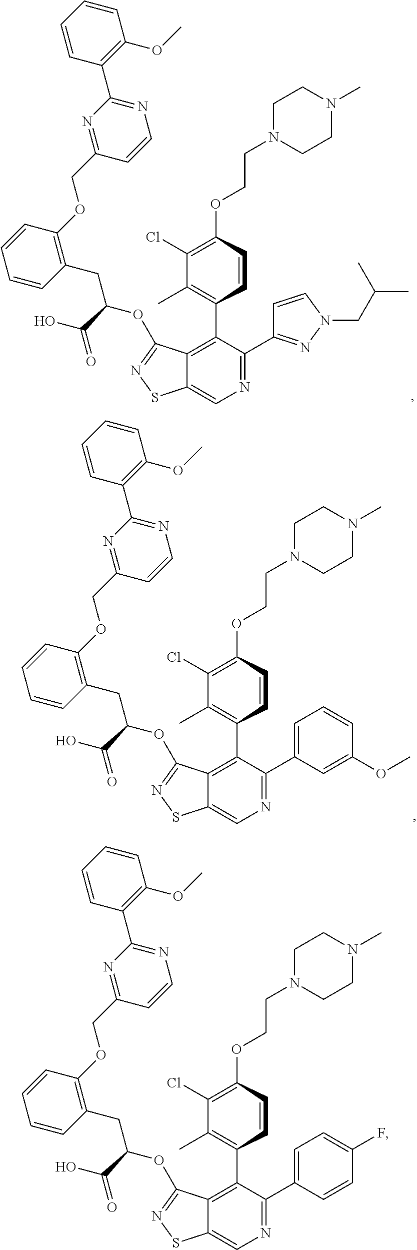 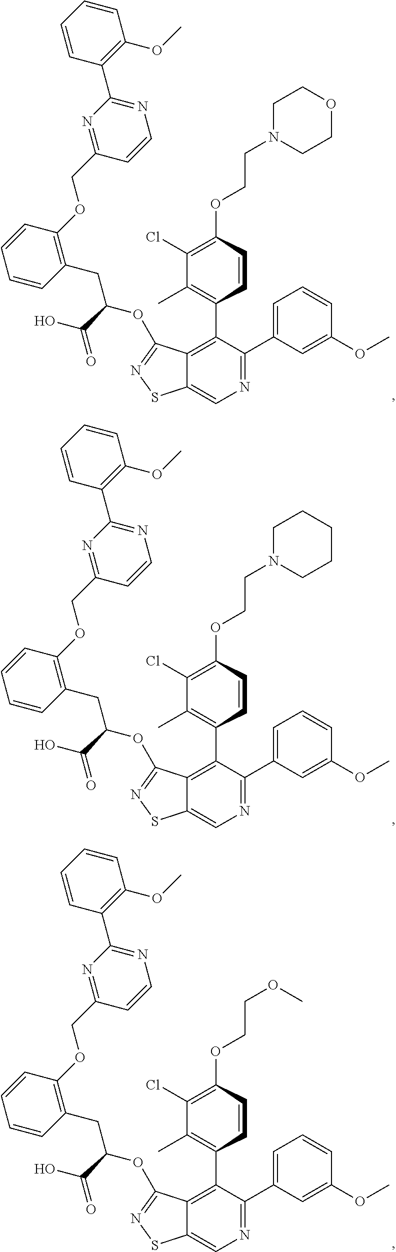 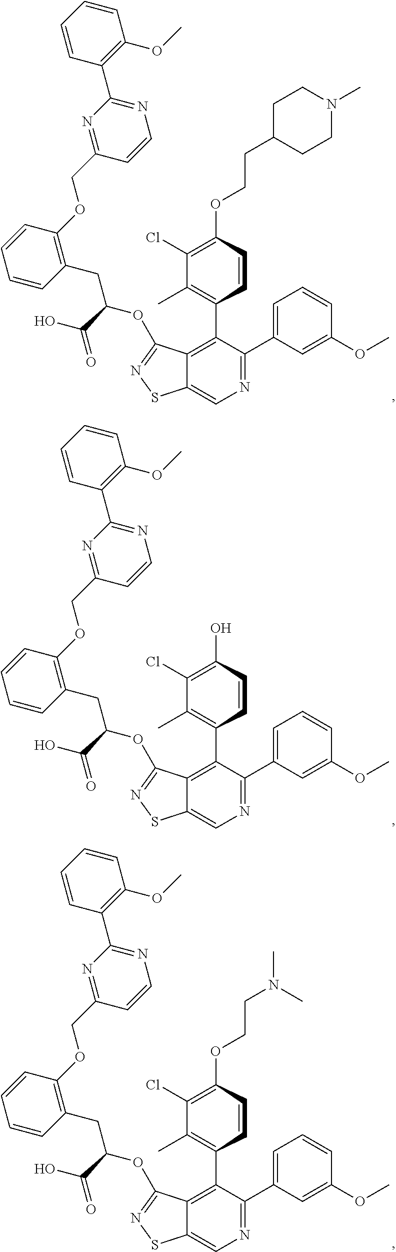 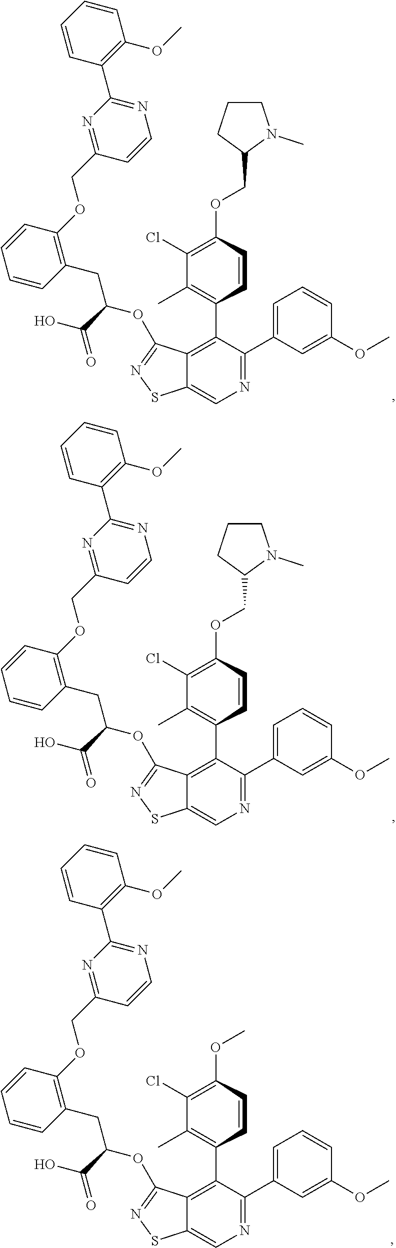 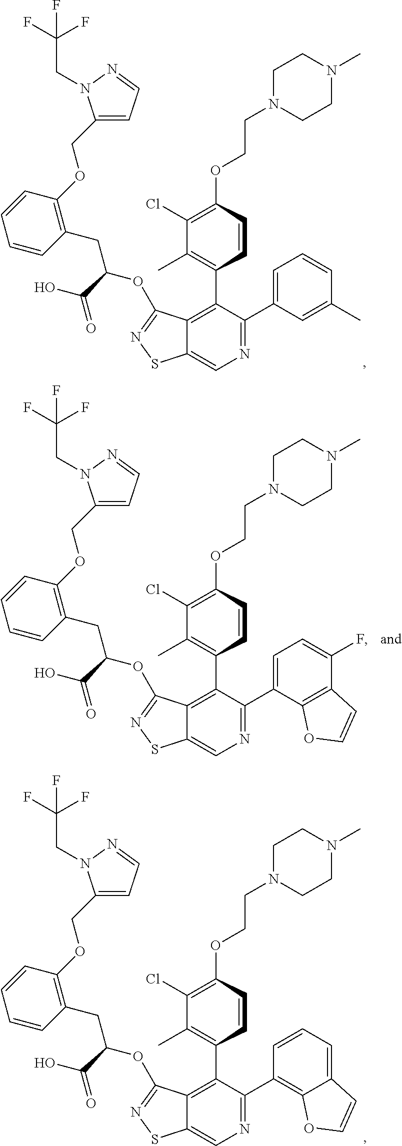
or a pharmaceutically acceptable salt thereof.

44. A compound selected from: 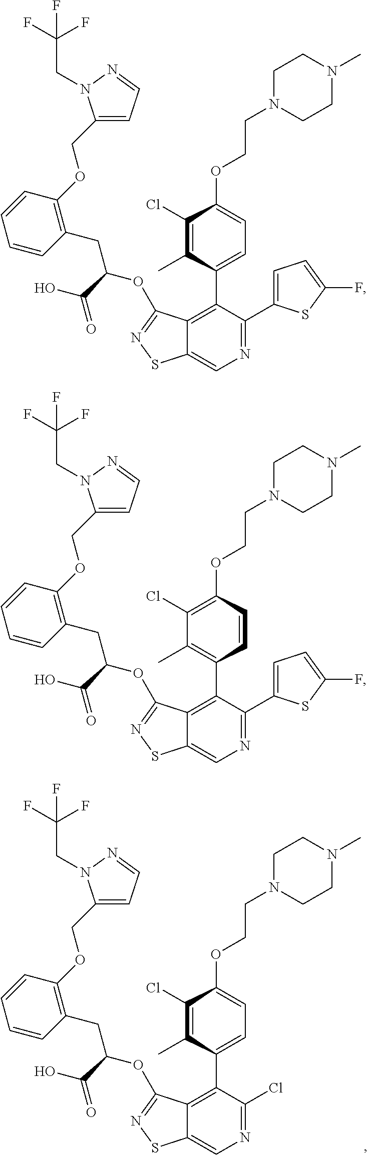 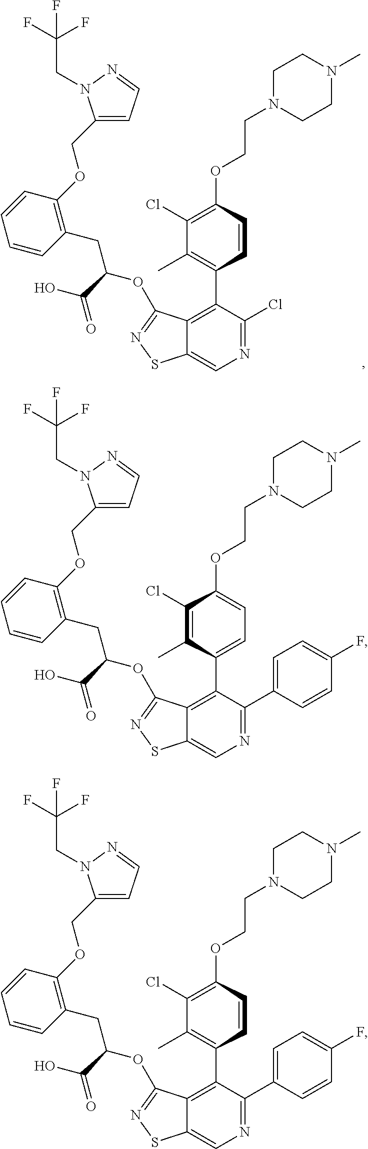 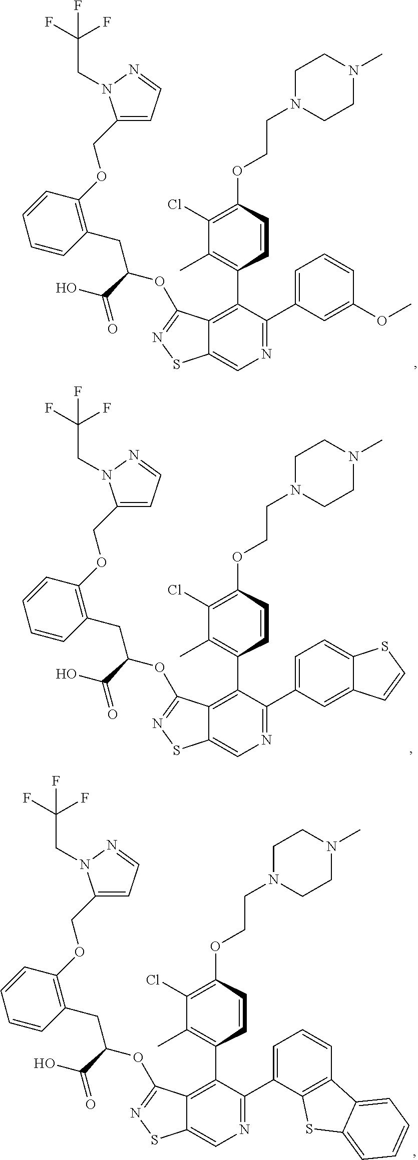 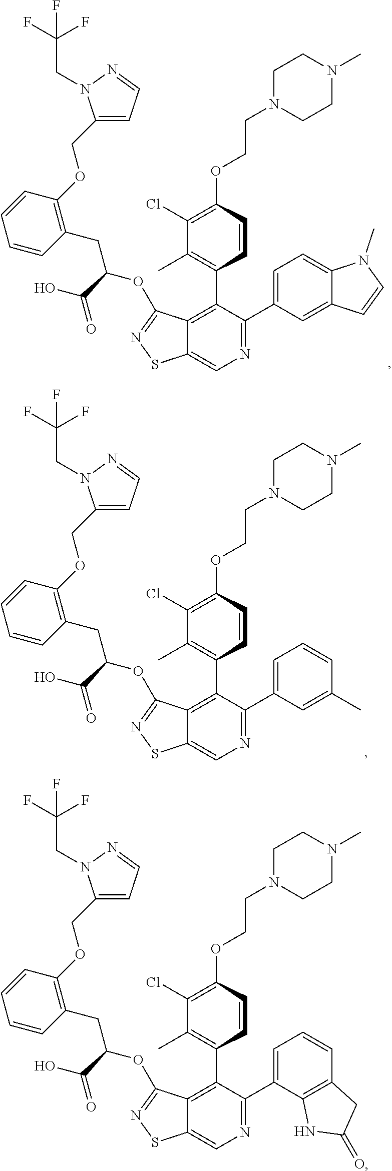 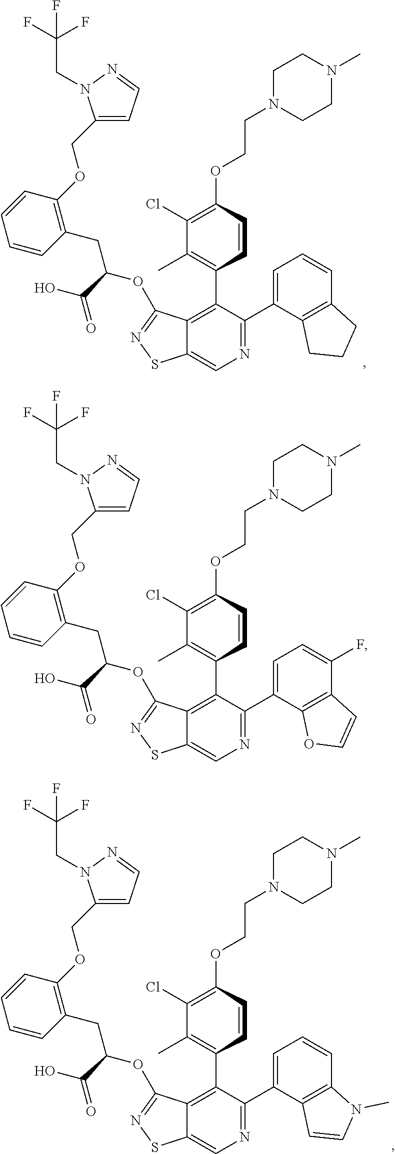 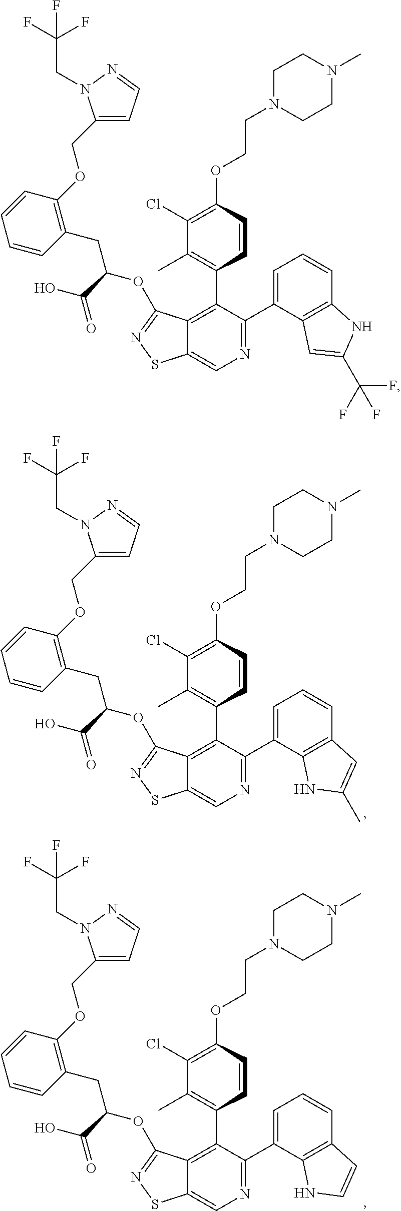 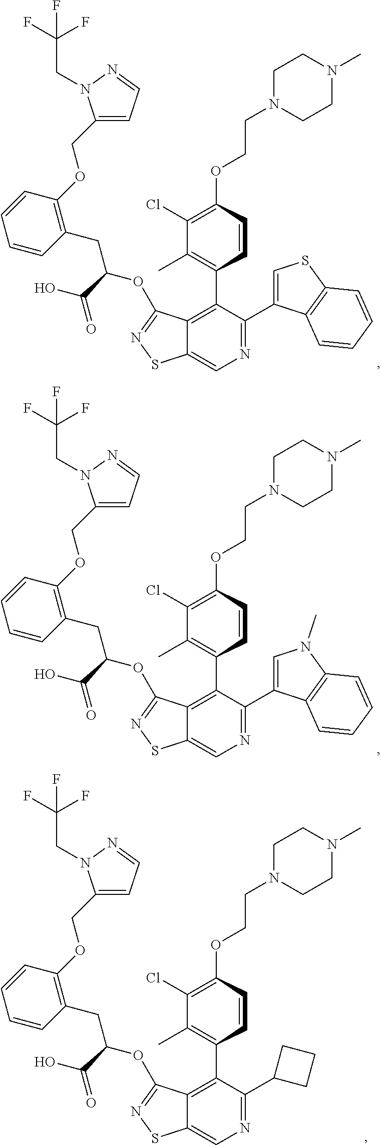 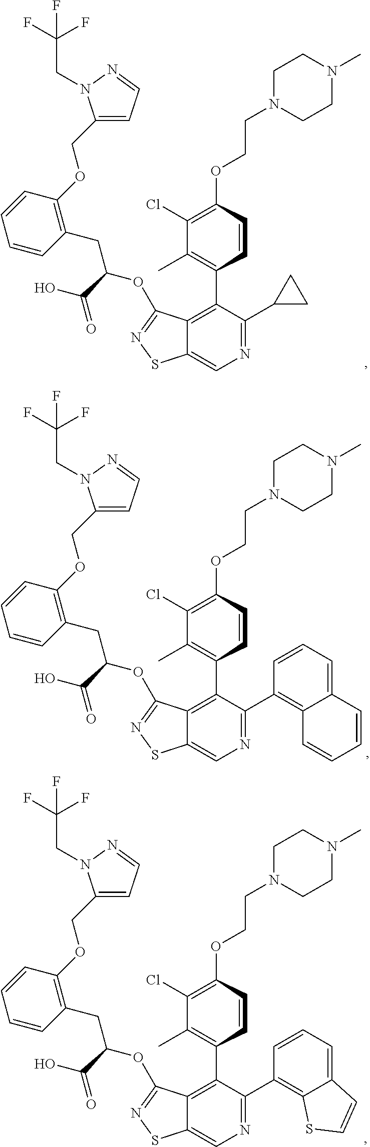 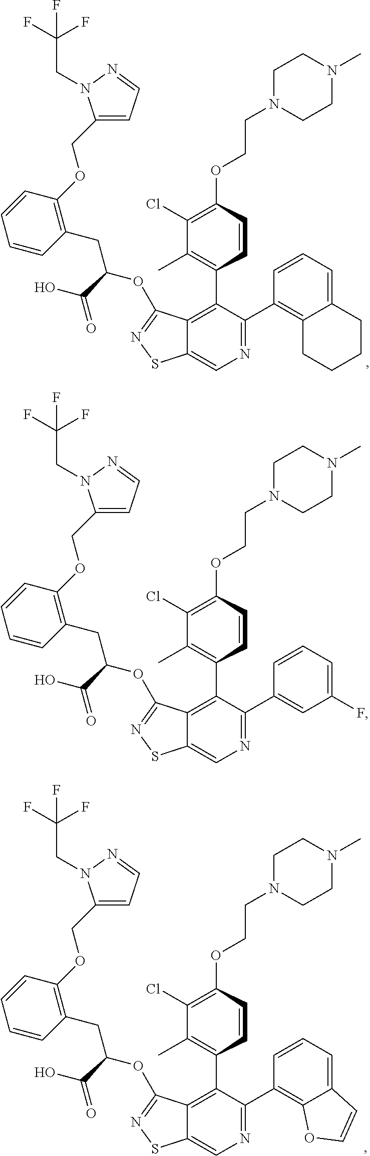 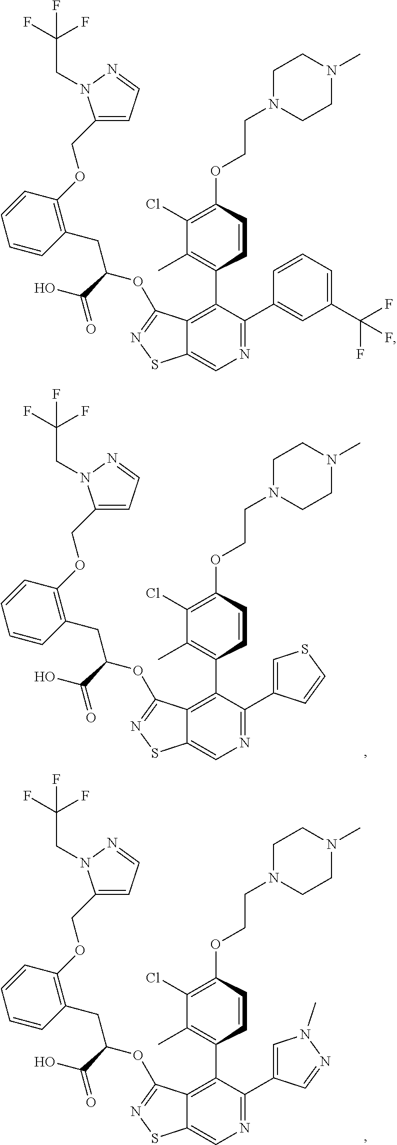 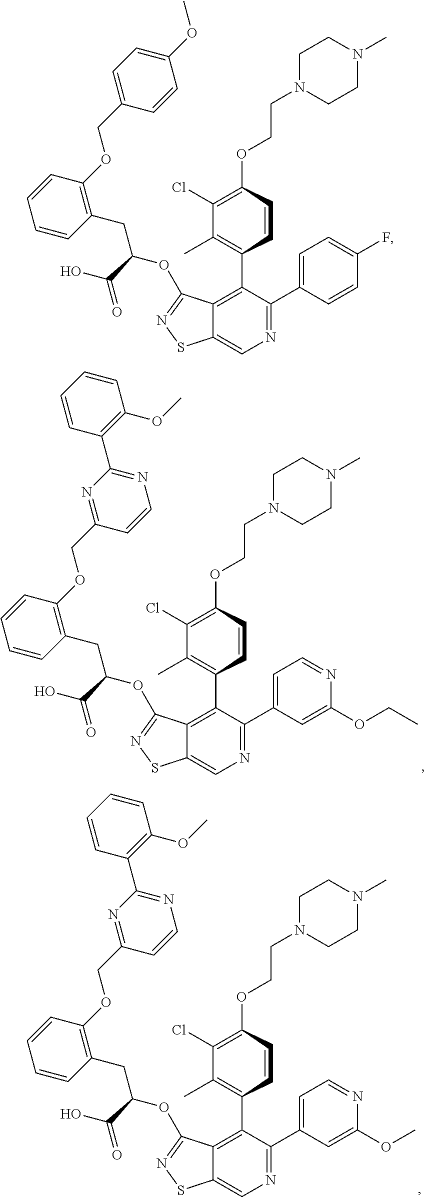 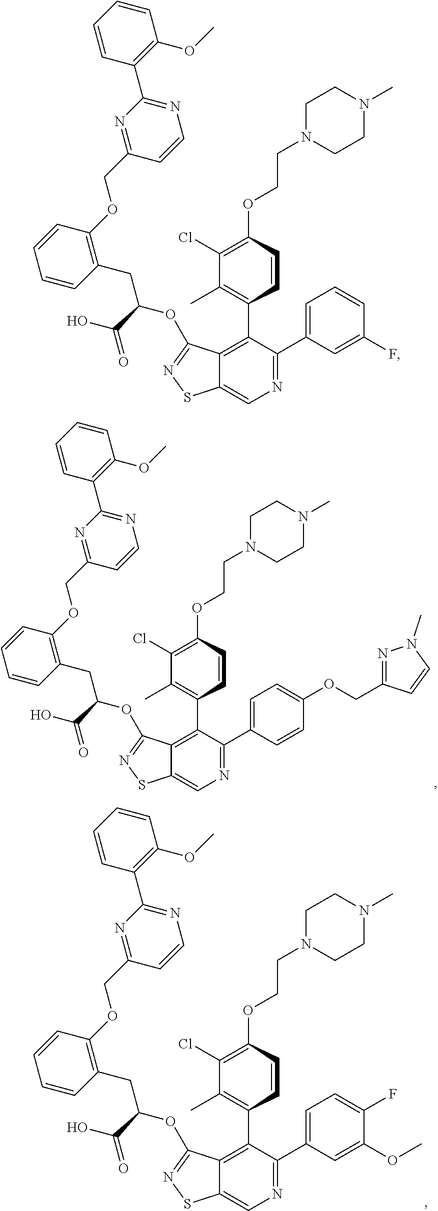 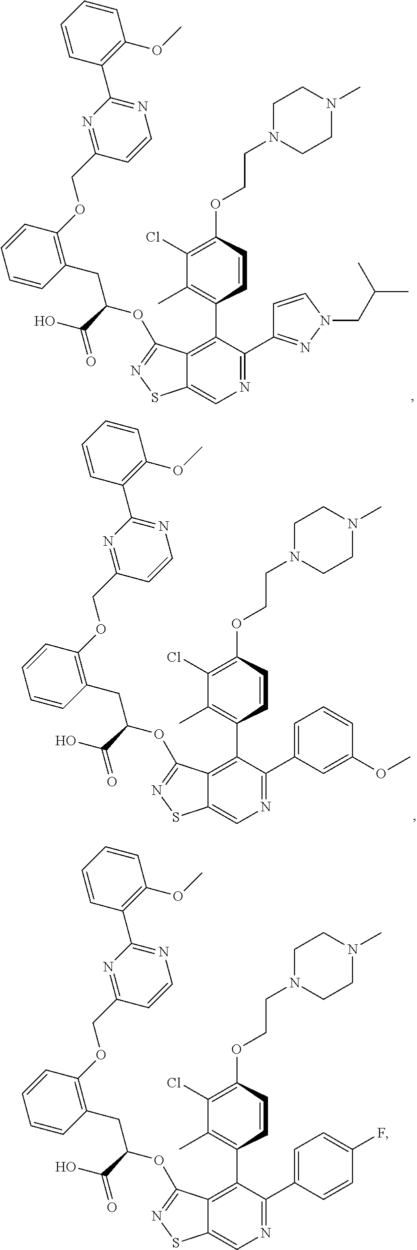 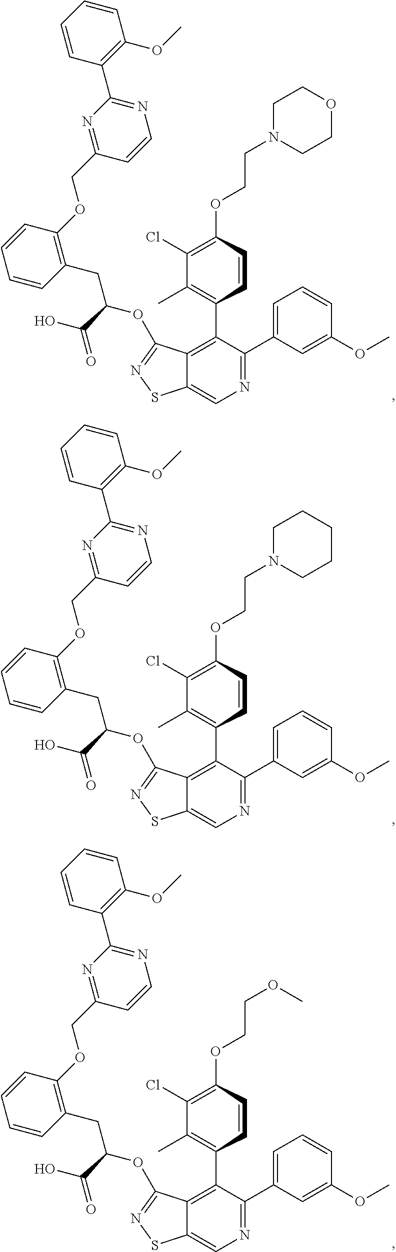 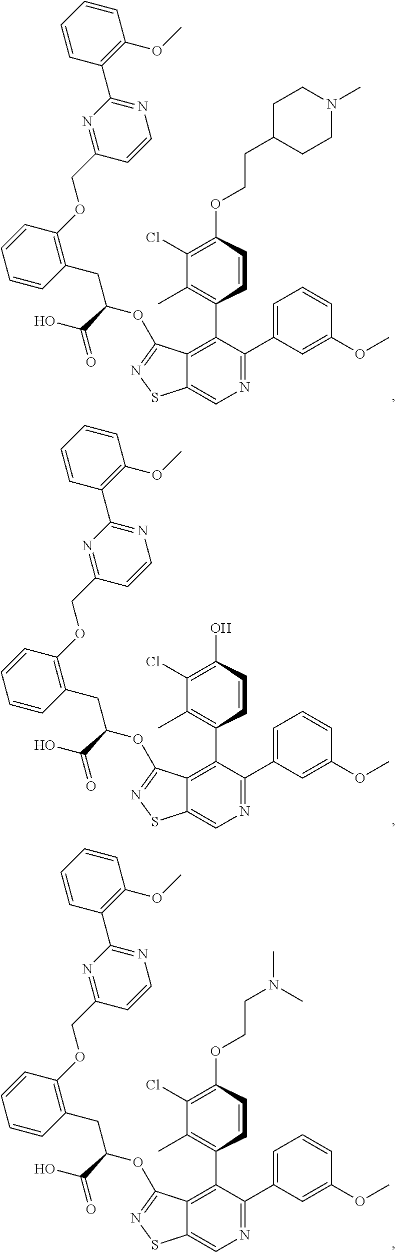 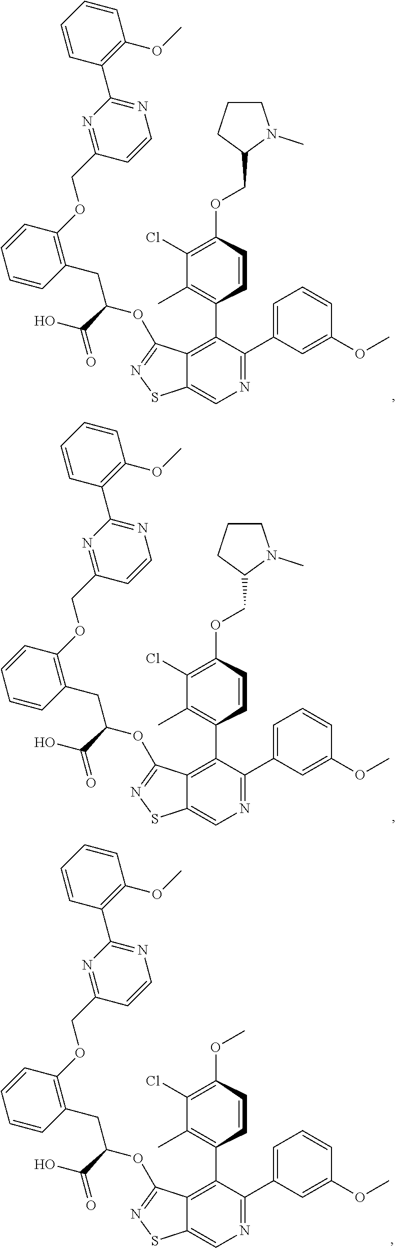 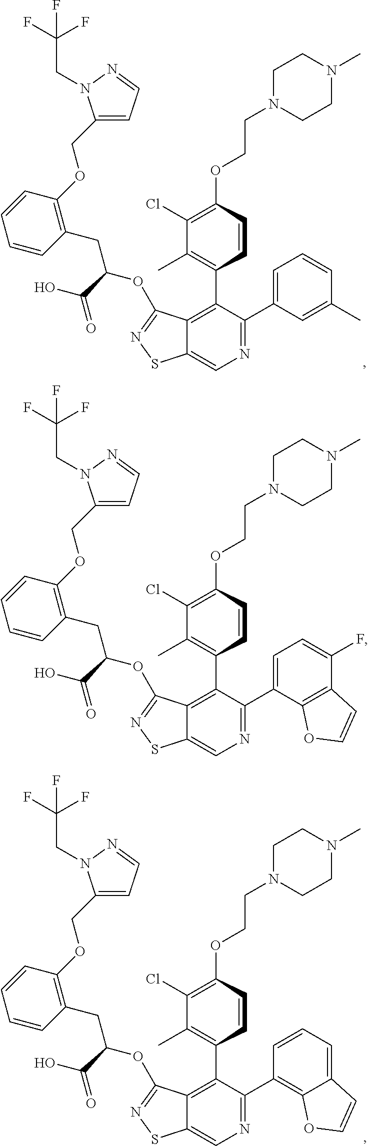 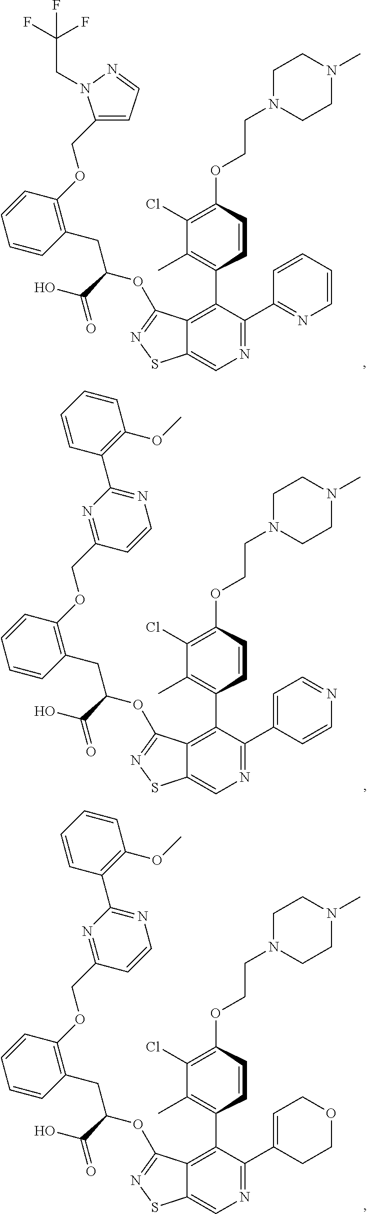 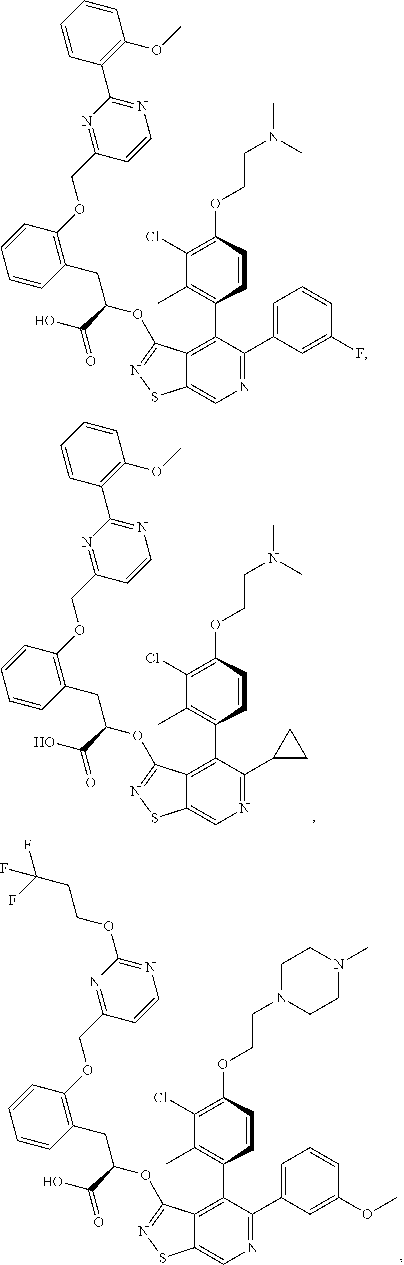 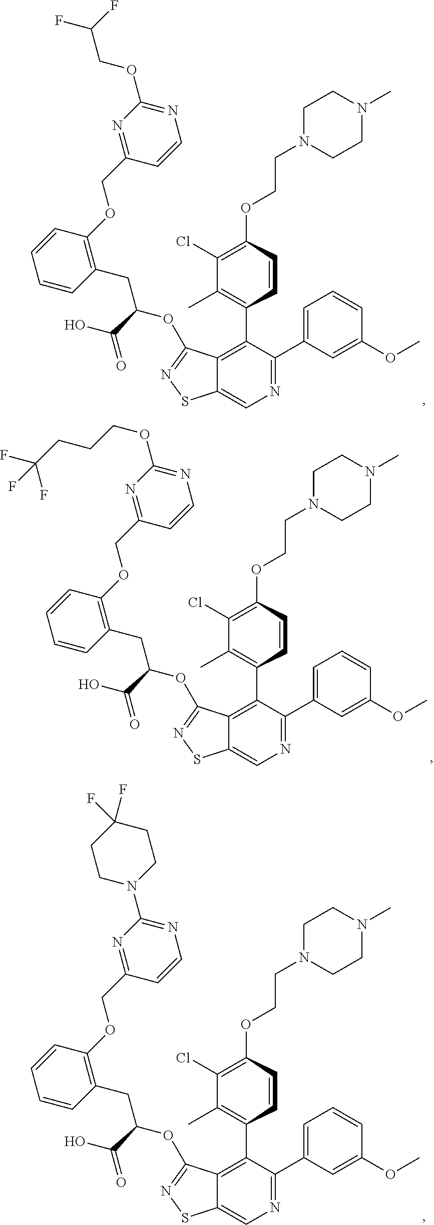 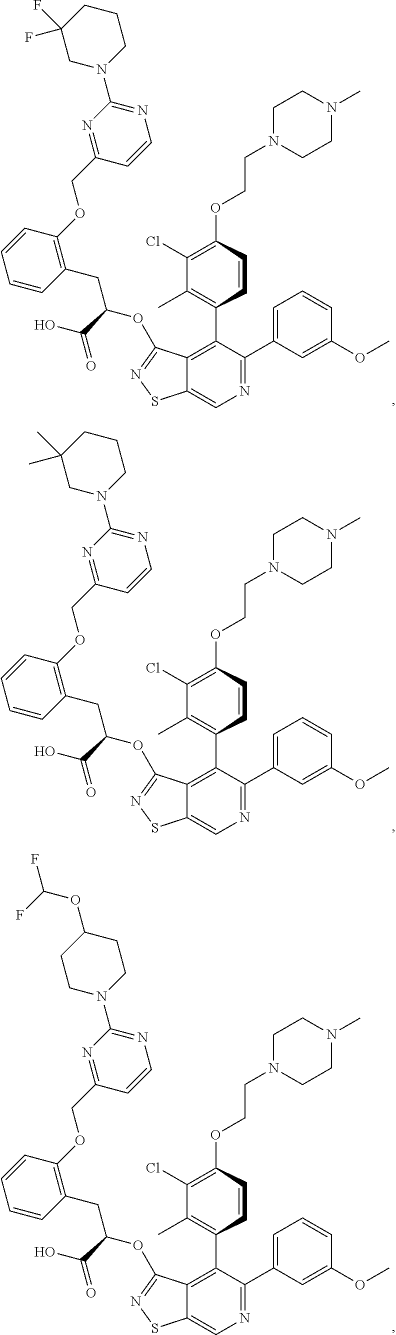 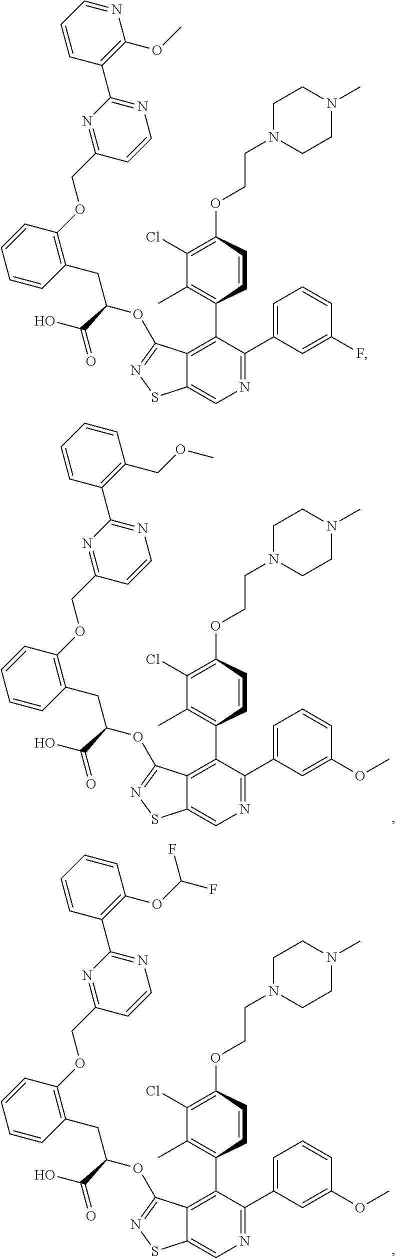 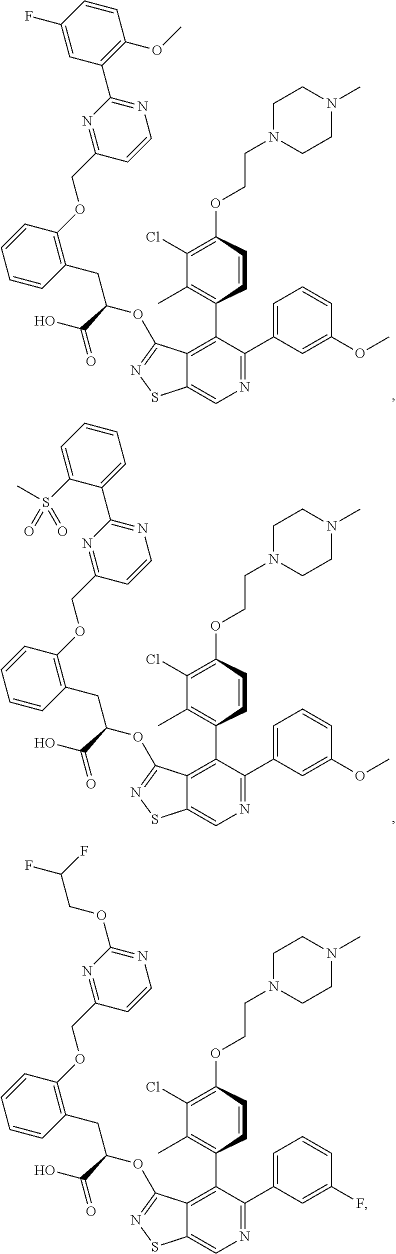 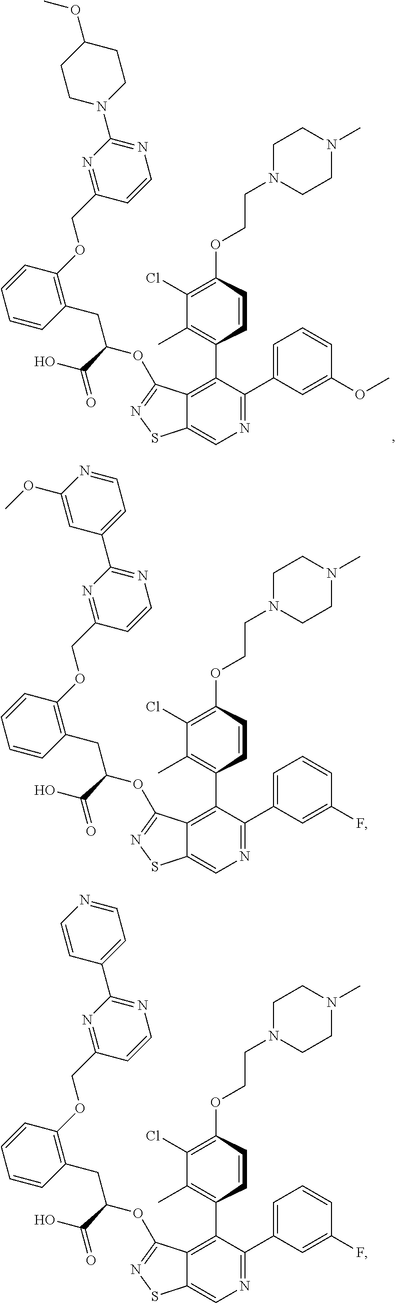 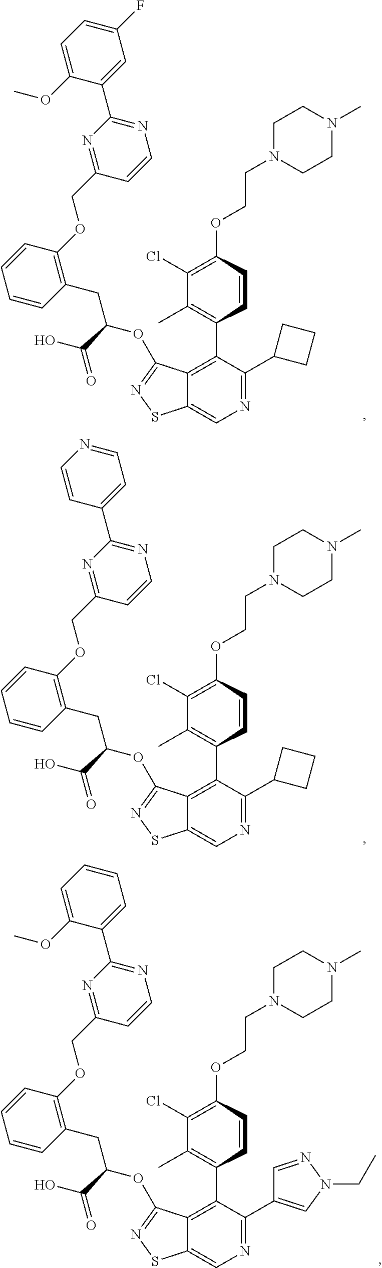 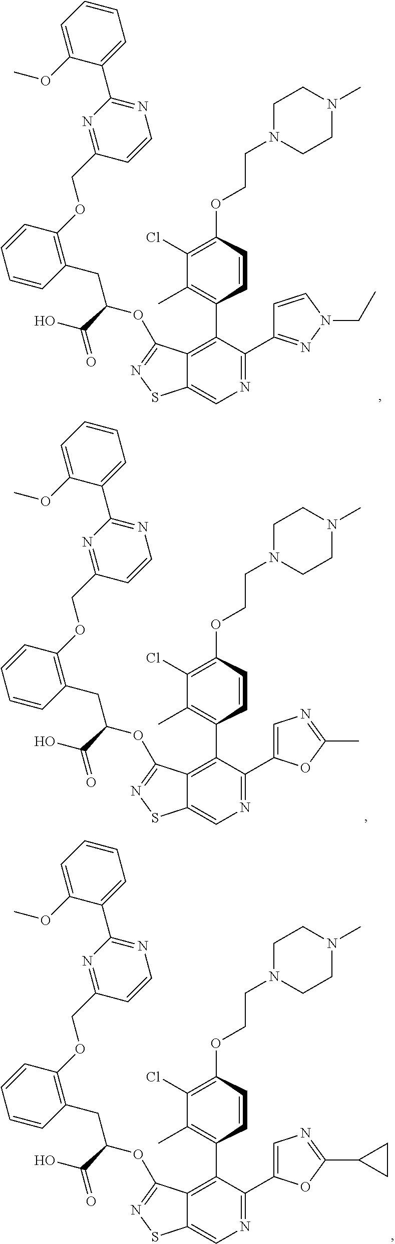 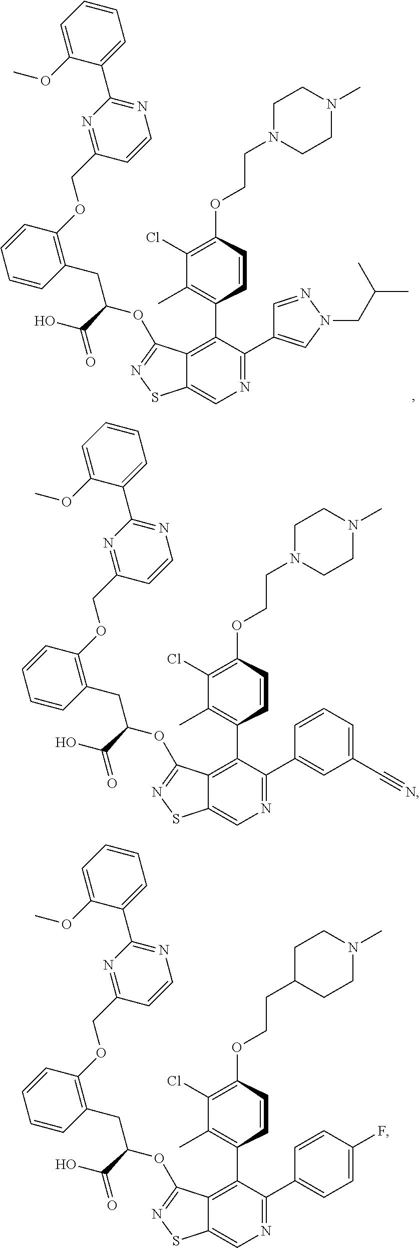 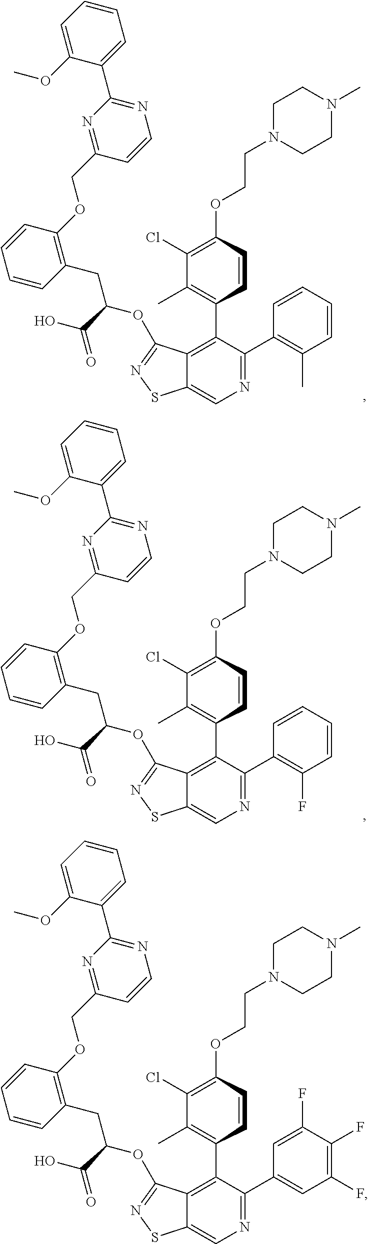 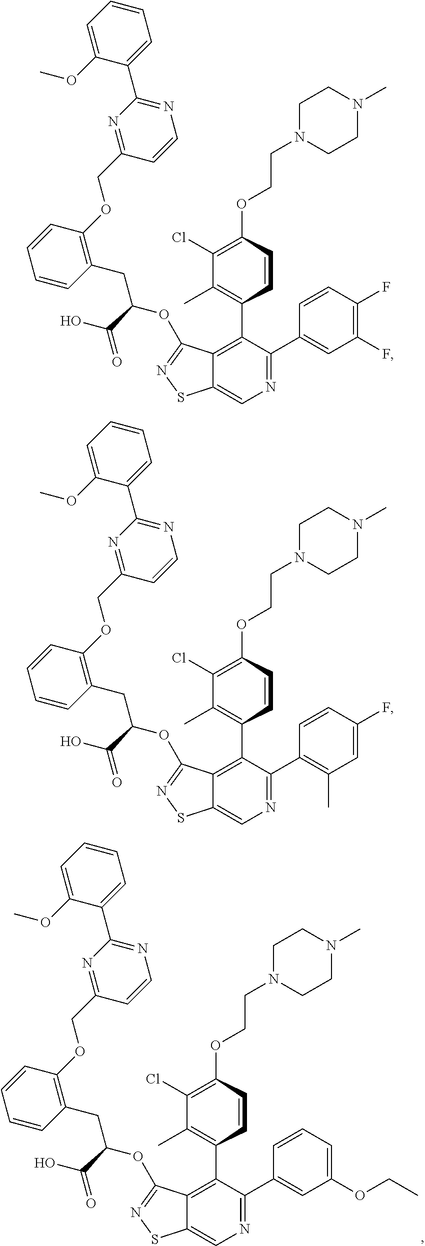 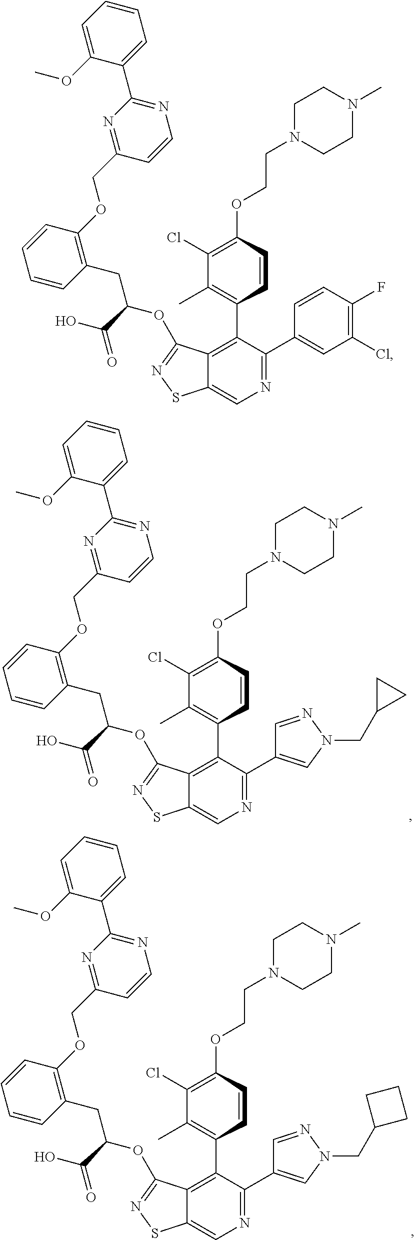 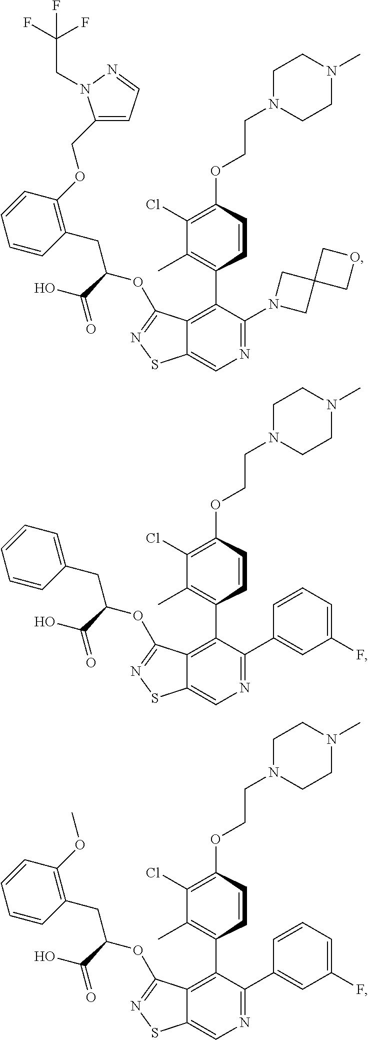 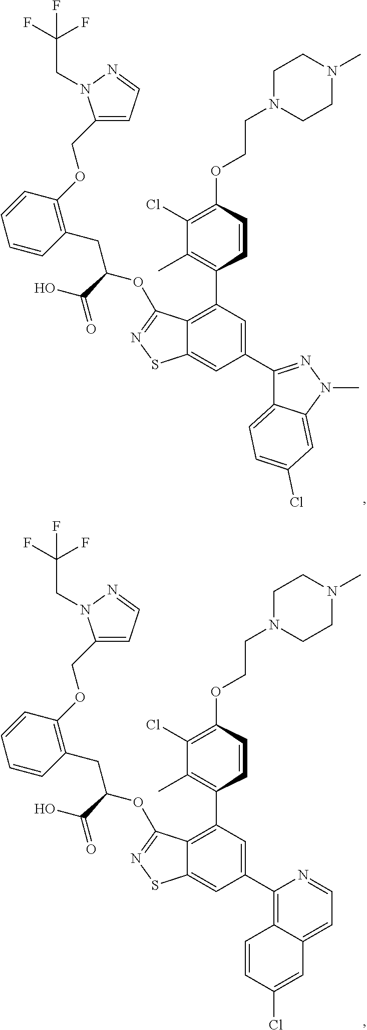 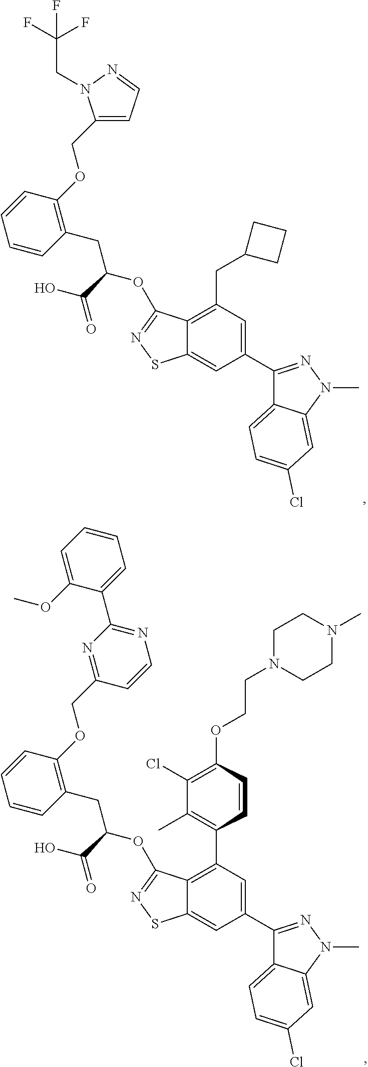 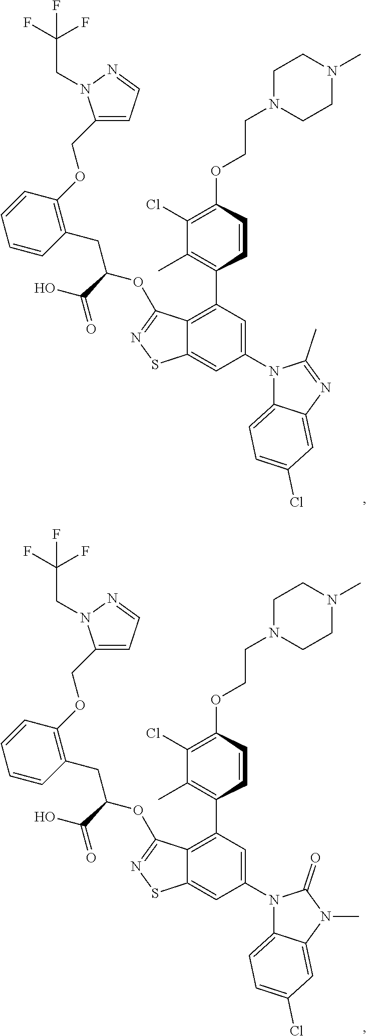 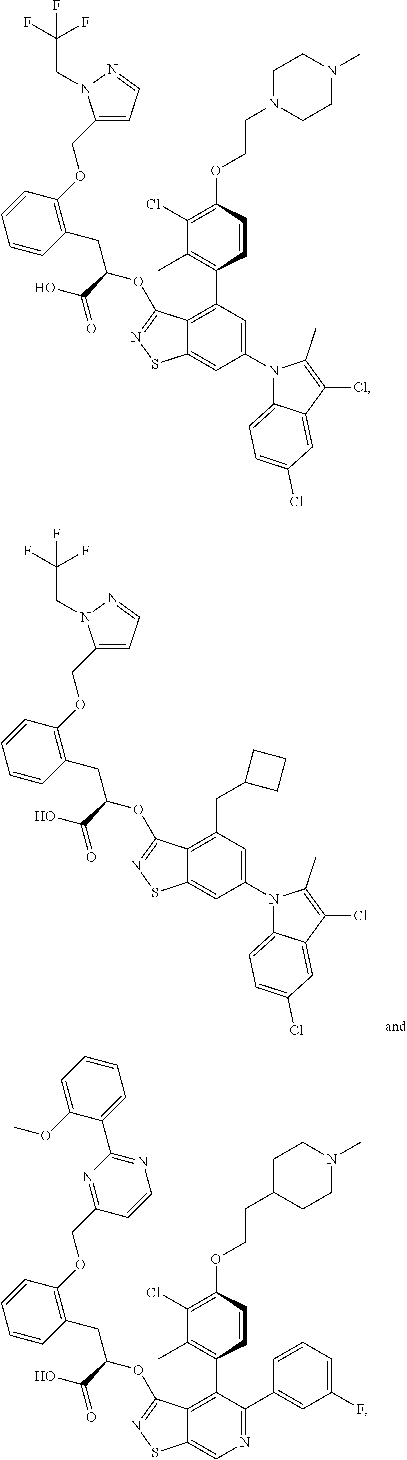
or a pharmaceutically acceptable salt thereof.

45. A compound selected from: 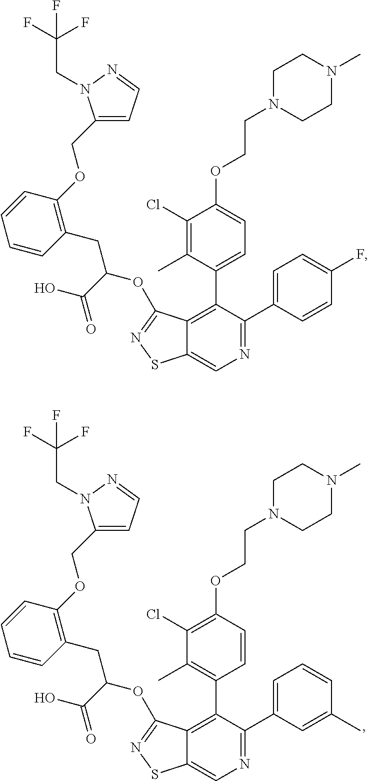 [SMILES: Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1] 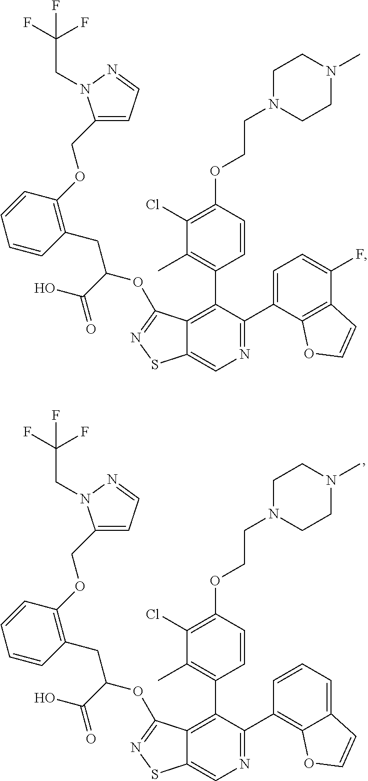 [SMILES: Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl] 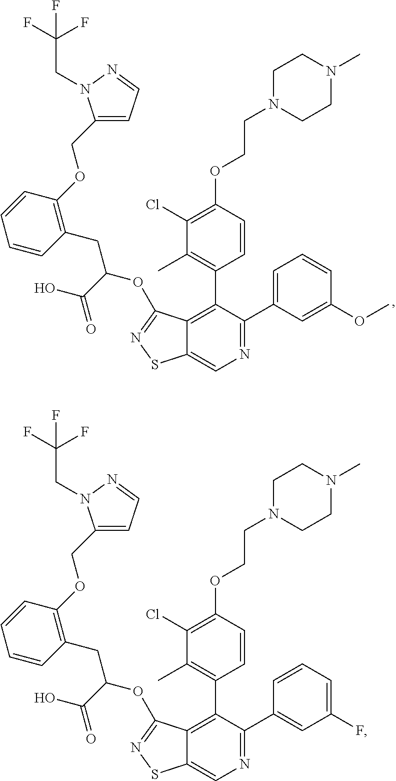 [SMILES: COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1 ; Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl] 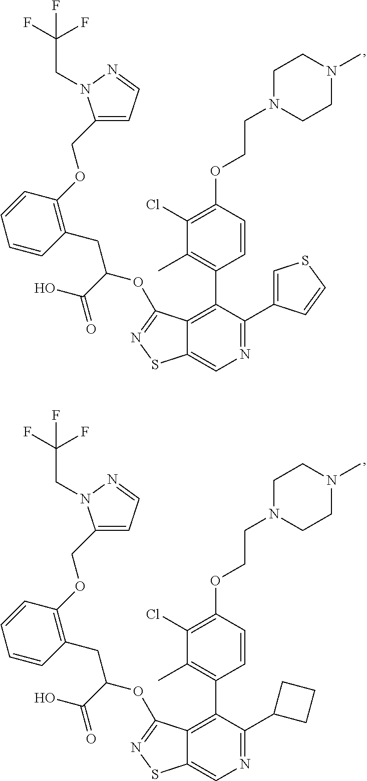 [SMILES: Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl] 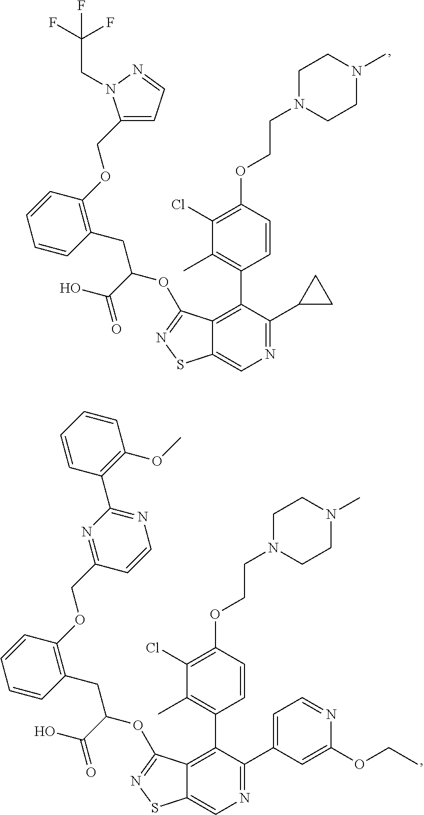 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1] 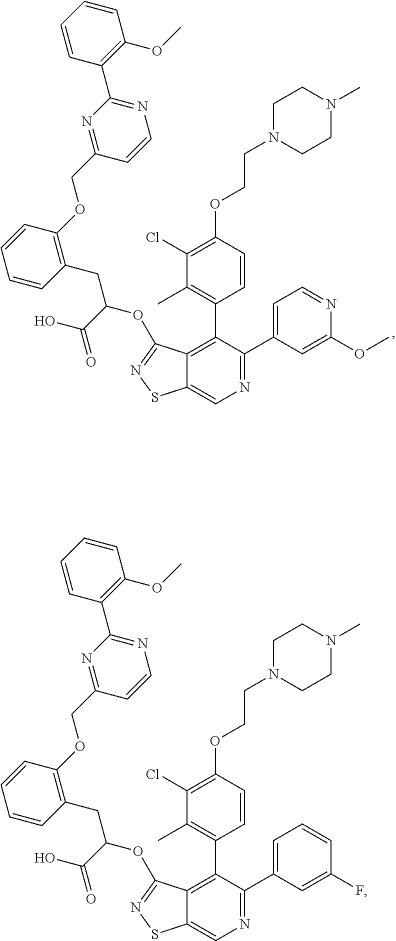 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1] 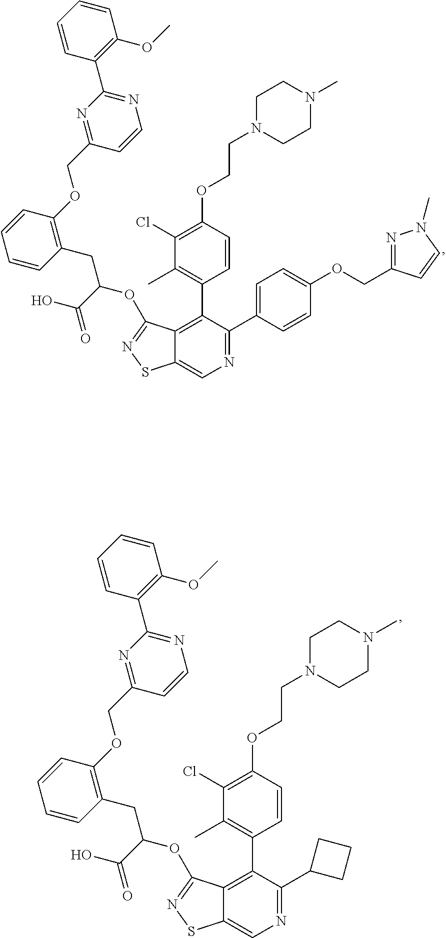 [SMILES: COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1] 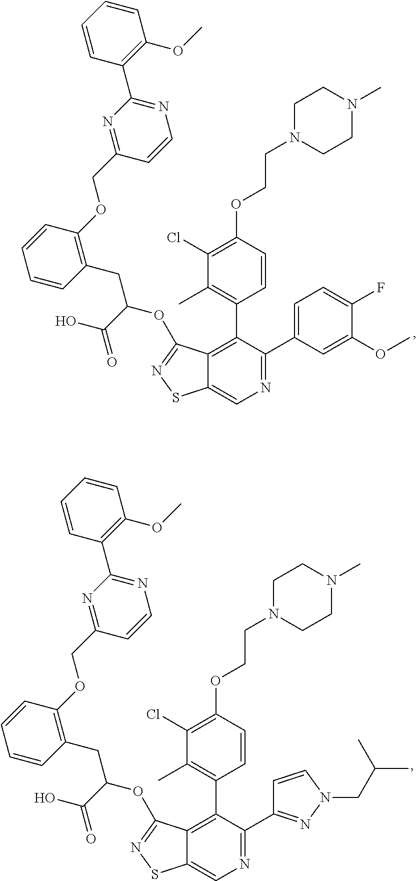 [SMILES: COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F ; COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1] 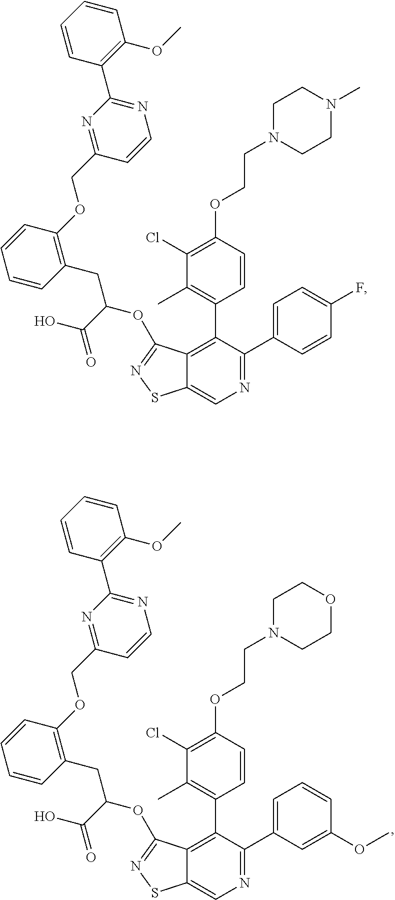 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1] 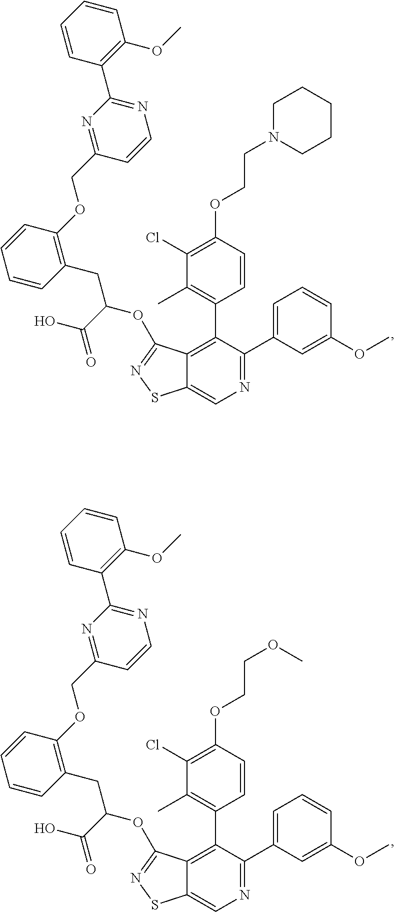 [SMILES: COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl] 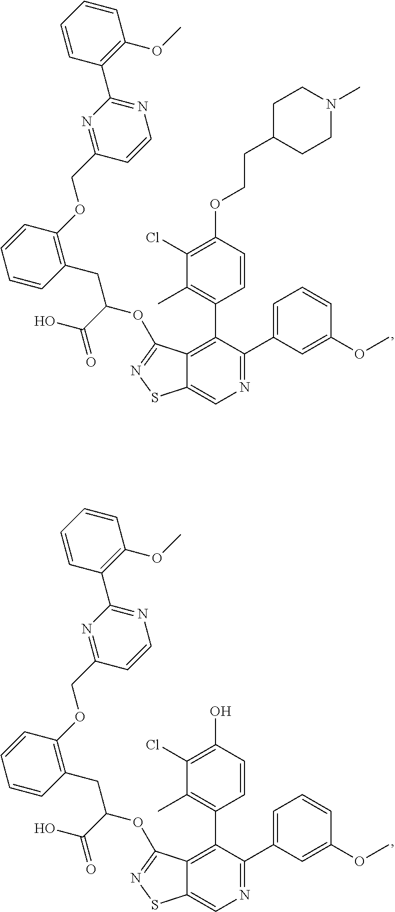 [SMILES: COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1] 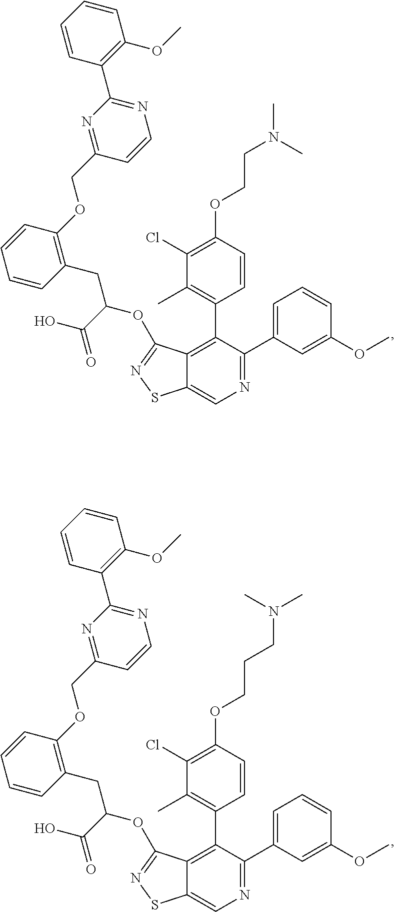 [SMILES: COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1] 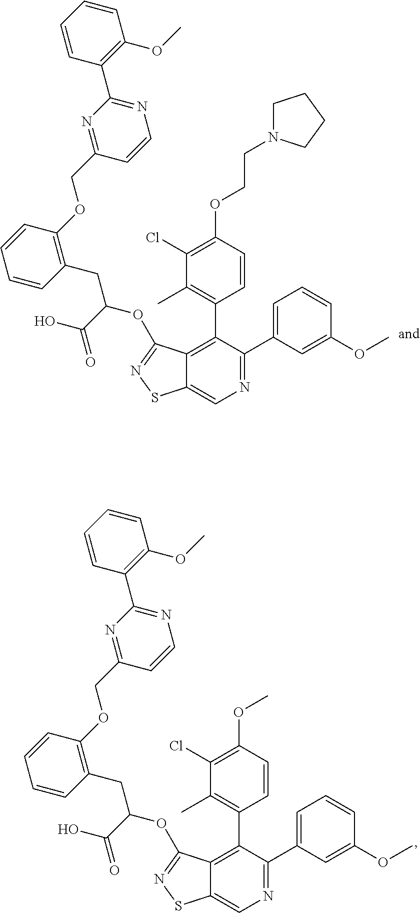 [SMILES: COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1 ; COc1cccc(-c2ncc3snc(OC(Cc4ccccc4OCc4ccnc(-c5ccccc5OC)n4)C(=O)O)c3c2-c2ccc(OC)c(Cl)c2C)c1]
or a pharmaceutically acceptable salt thereof.

46. A compound selected from: 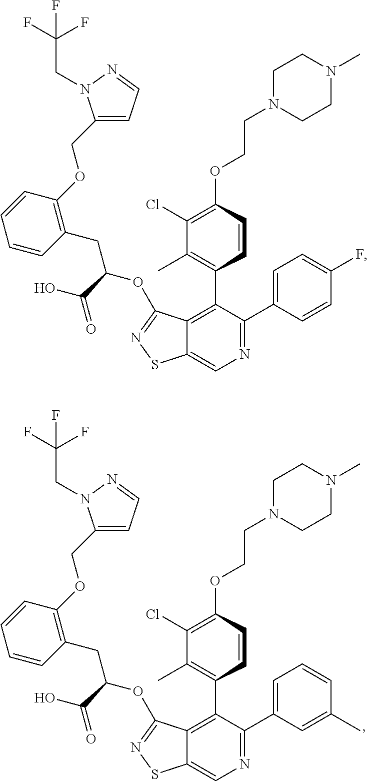 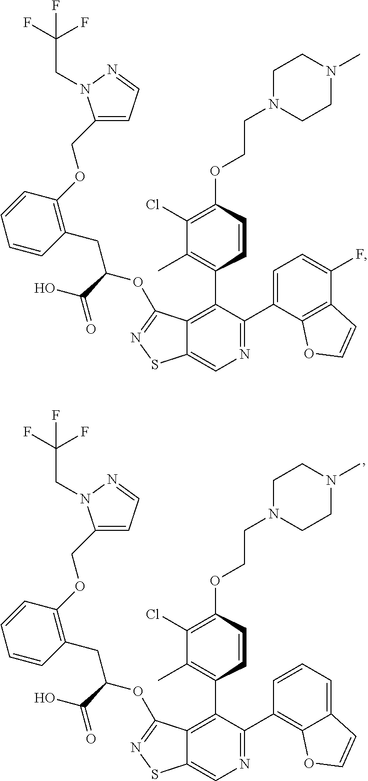 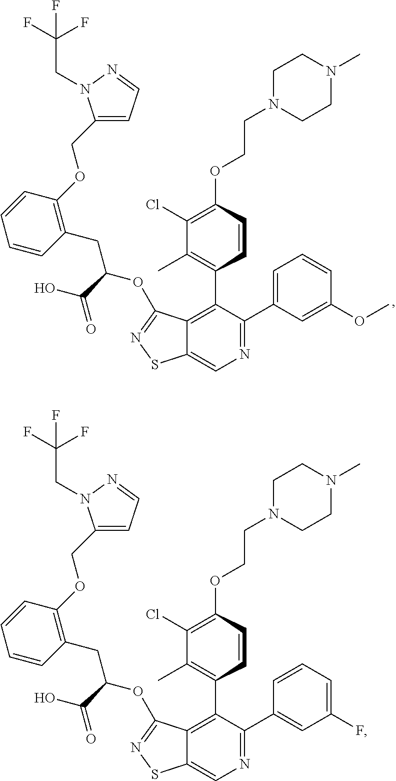 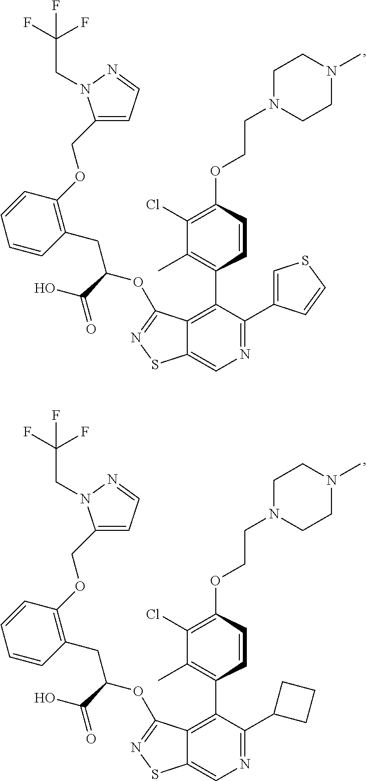 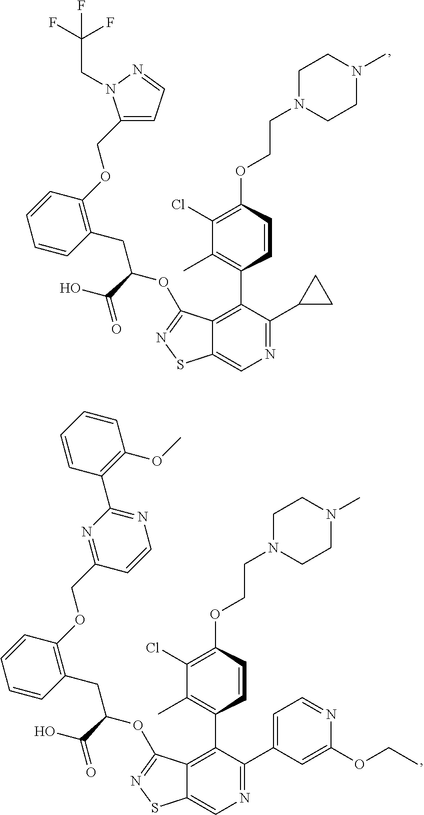 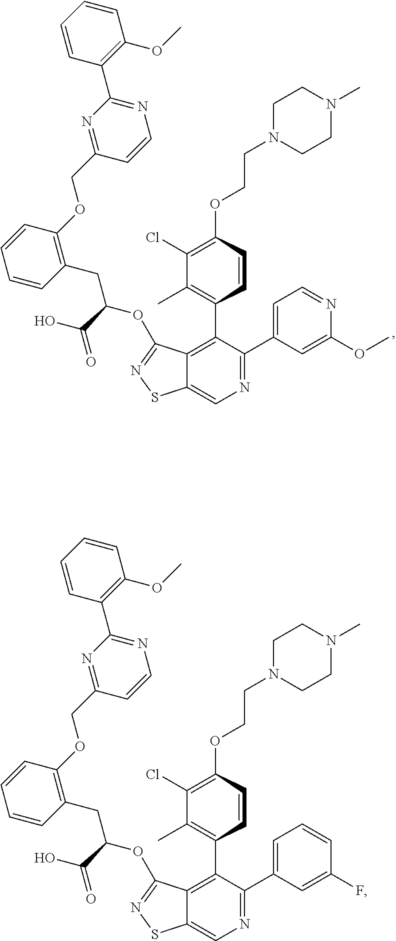 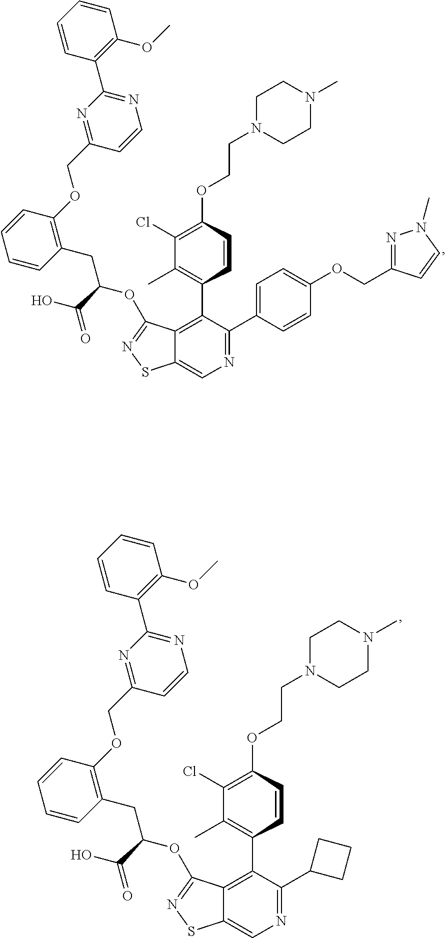 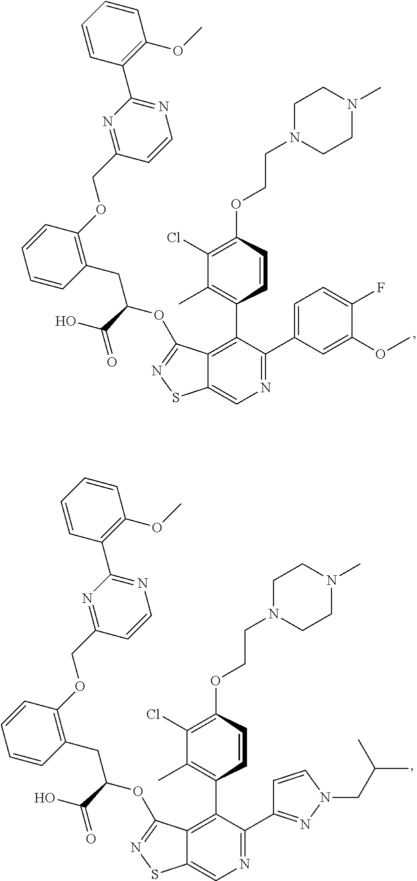 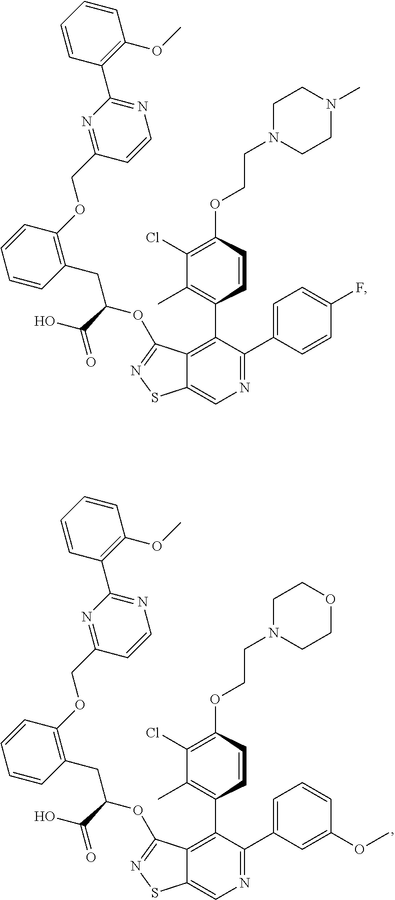 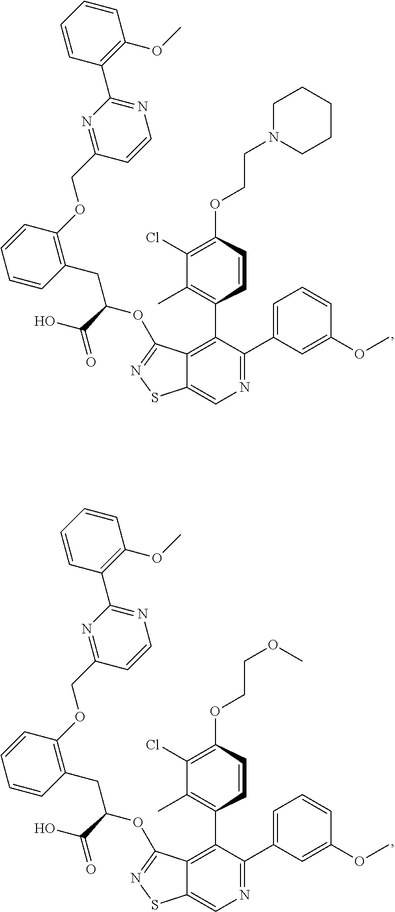 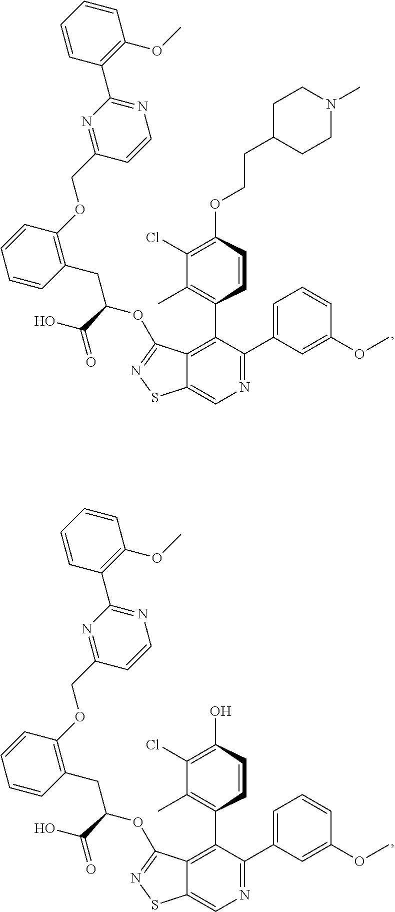 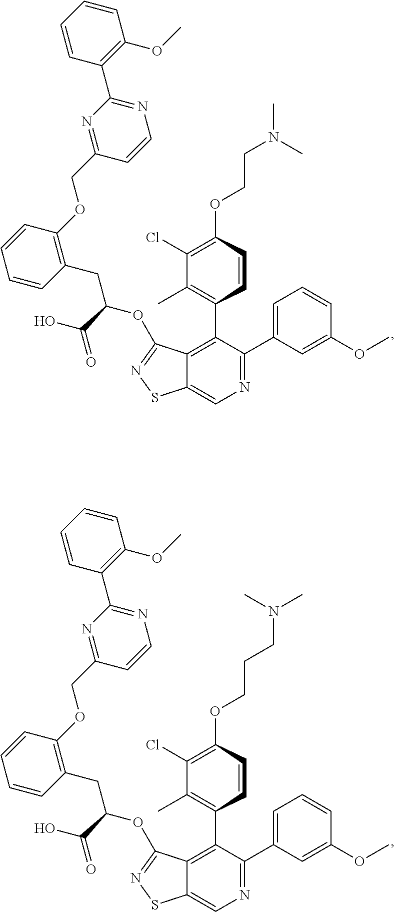 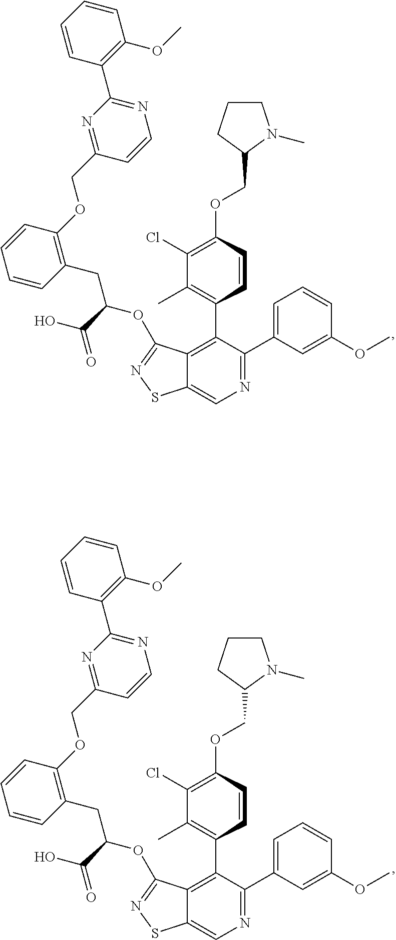 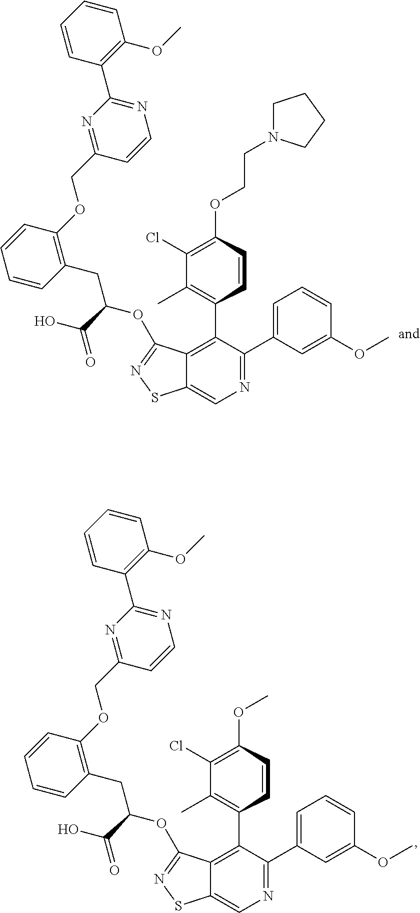
or a pharmaceutically acceptable salt thereof.

47. A compound selected from: 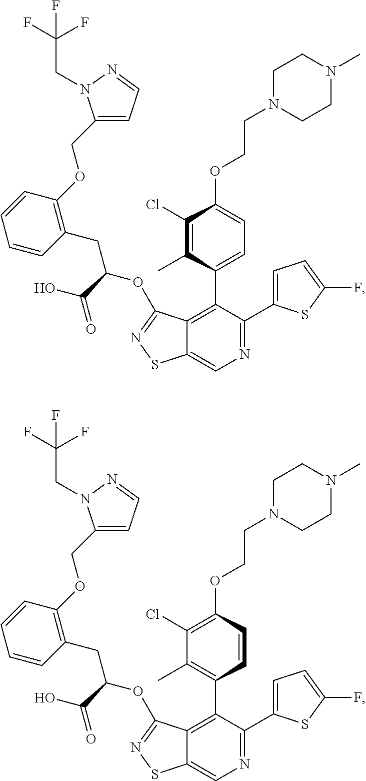 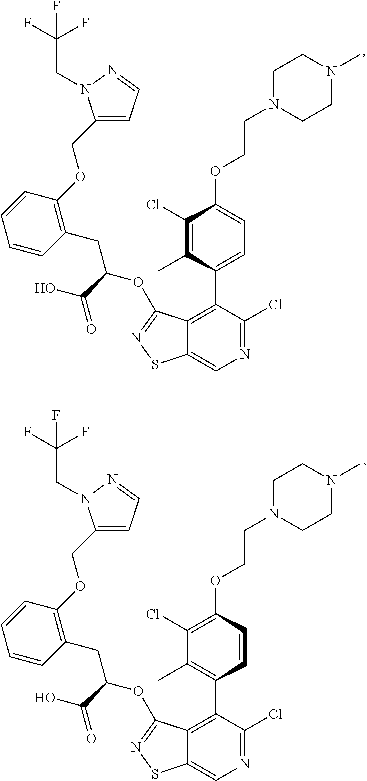 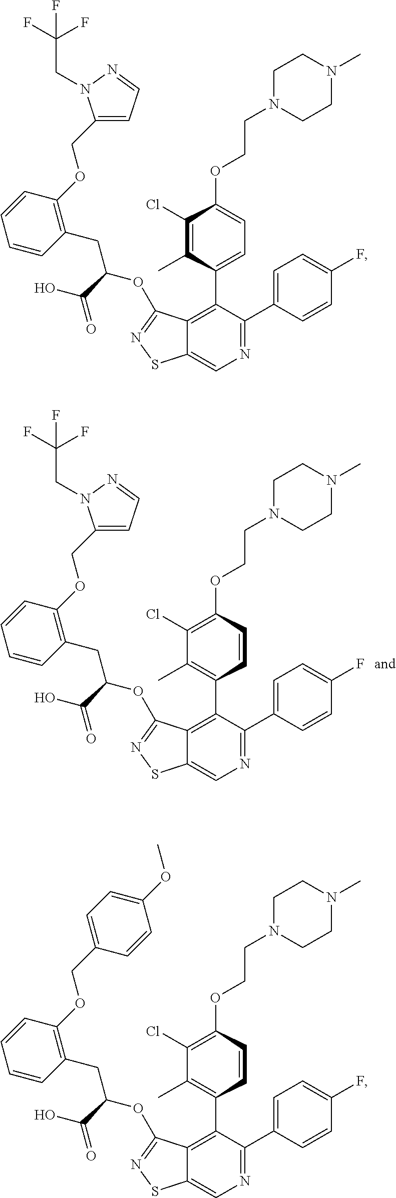
or a pharmaceutically acceptable salt thereof.

48. A compound selected from: 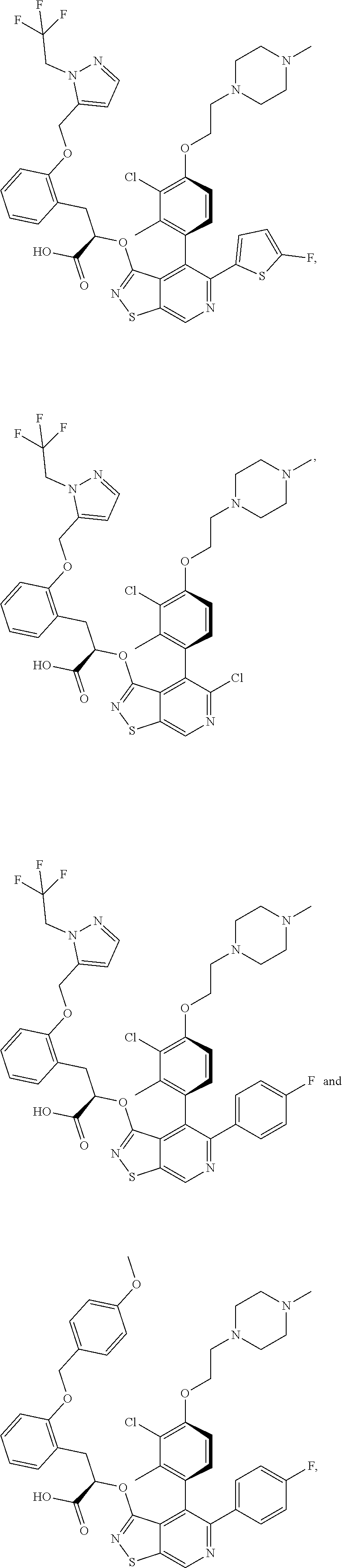
or a pharmaceutically acceptable salt thereof.

49. A compound selected from: 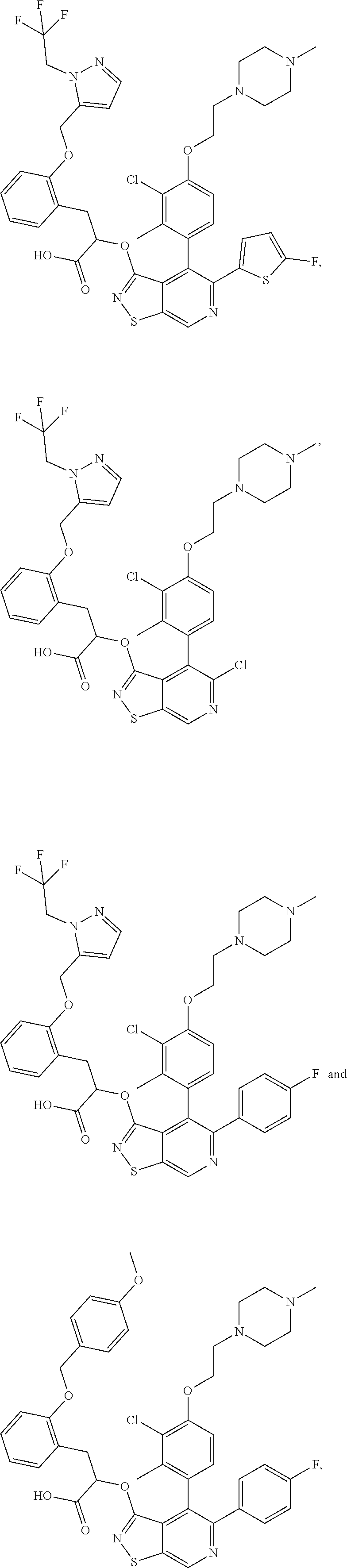 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl ; COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1]
or a pharmaceutically acceptable salt thereof.

50. A compound selected from: 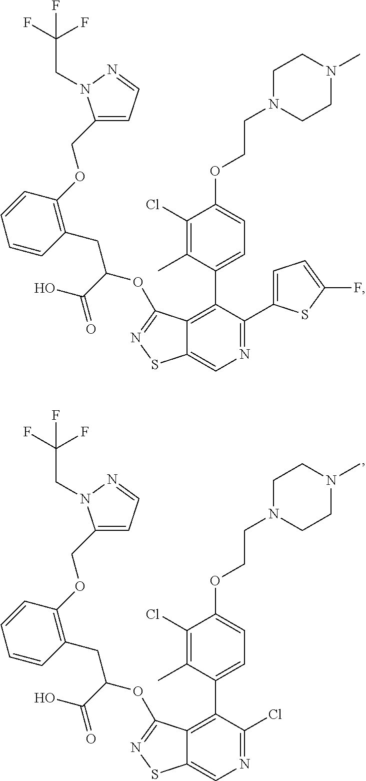 [SMILES: Cc1c(-c2c(-c3ccc(F)s3)ncc3snc(OC(Cc4ccccc4OCc4ccnn4CC(F)(F)F)C(=O)O)c23)ccc(OCCN2CCN(C)CC2)c1Cl ; Cc1c(-c2c(Cl)ncc3snc(OC(Cc4ccccc4OCc4ccnn4CC(F)(F)F)C(=O)O)c23)ccc(OCCN2CCN(C)CC2)c1Cl] 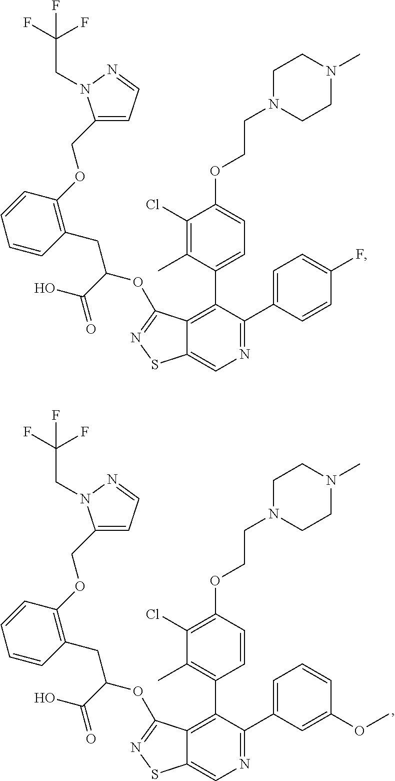 [SMILES: Cc1c(-c2c(-c3ccc(F)cc3)ncc3snc(OC(Cc4ccccc4OCc4ccnn4CC(F)(F)F)C(=O)O)c23)ccc(OCCN2CCN(C)CC2)c1Cl ; COc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1] 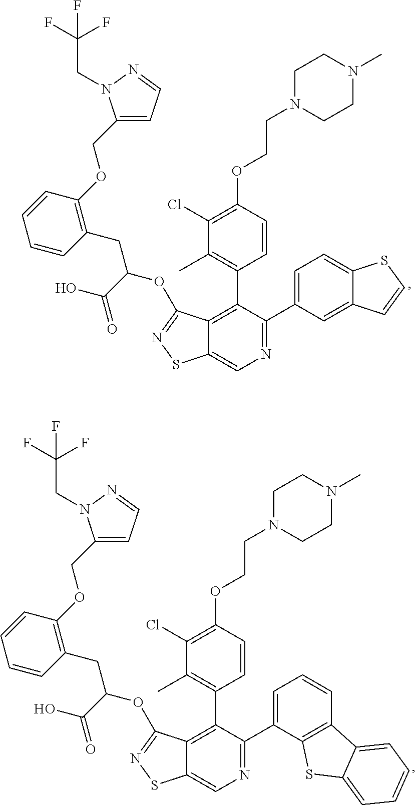 [SMILES: Cc1c(-c2c(-c3ccc4sccc4c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3sc3ccccc34)ncc3snc(OC(Cc4ccccc4OCc4ccnn4CC(F)(F)F)C(=O)O)c23)ccc(OCCN2CCN(C)CC2)c1Cl] 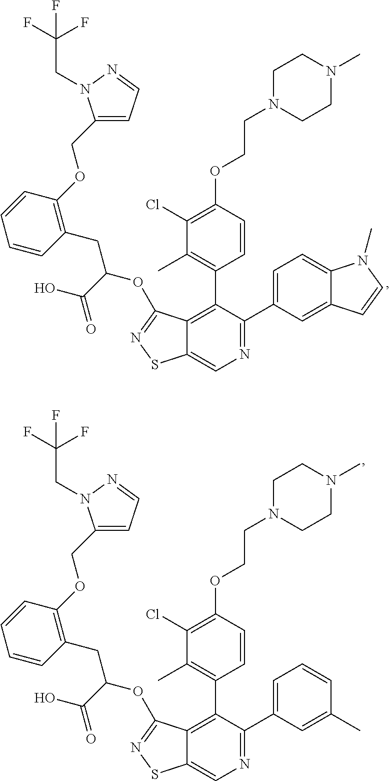 [SMILES: Cc1c(-c2c(-c3ccc4c(ccn4C)c3)ncc3snc(OC(Cc4ccccc4OCc4ccnn4CC(F)(F)F)C(=O)O)c23)ccc(OCCN2CCN(C)CC2)c1Cl ; Cc1cccc(-c2ncc3snc(OC(Cc4ccccc4OCc4ccnn4CC(F)(F)F)C(=O)O)c3c2-c2ccc(OCCN3CCN(C)CC3)c(Cl)c2C)c1] 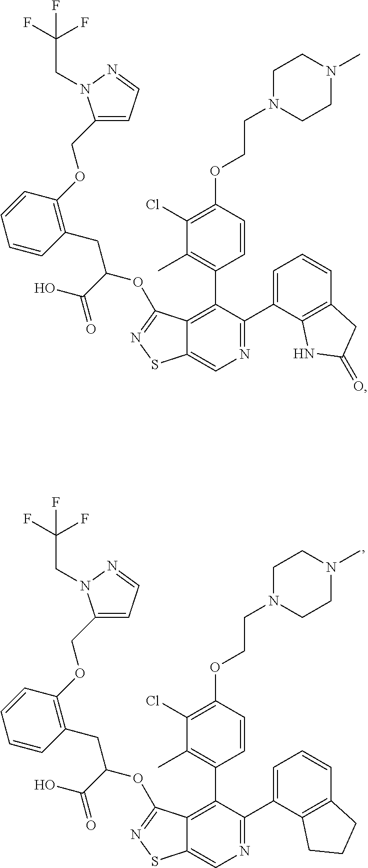 [SMILES: Cc1c(-c2c(-c3cccc4c3NC(=O)C4)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3CCC4)ncc3snc(OC(Cc4ccccc4OCc4ccnn4CC(F)(F)F)C(=O)O)c23)ccc(OCCN2CCN(C)CC2)c1Cl] 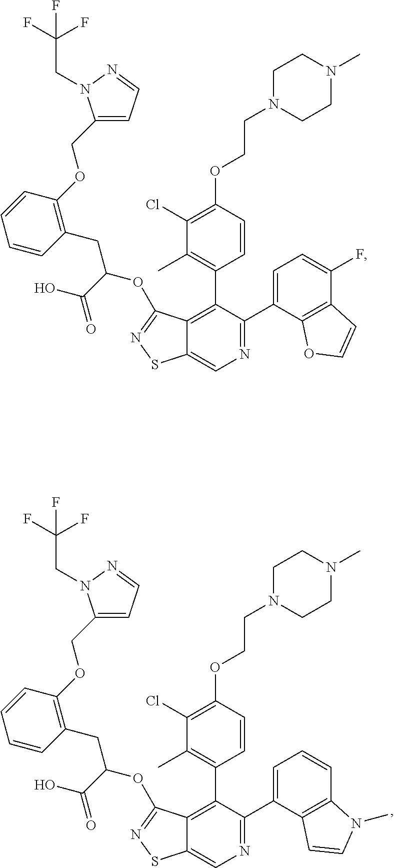 [SMILES: Cc1c(-c2c(-c3ccc(F)c4ccoc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3ccn4C)ncc3snc(OC(Cc4ccccc4OCc4ccnn4CC(F)(F)F)C(=O)O)c23)ccc(OCCN2CCN(C)CC2)c1Cl] 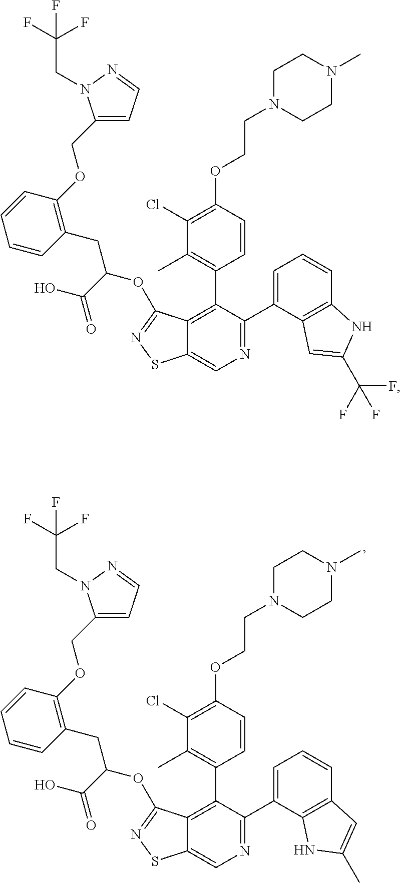 [SMILES: Cc1c(-c2c(-c3cccc4[nH]c(C(F)(F)F)cc34)ncc3snc(OC(Cc4ccccc4OCc4ccnn4CC(F)(F)F)C(=O)O)c23)ccc(OCCN2CCN(C)CC2)c1Cl ; Cc1cc2cccc(-c3ncc4snc(OC(Cc5ccccc5OCc5ccnn5CC(F)(F)F)C(=O)O)c4c3-c3ccc(OCCN4CCN(C)CC4)c(Cl)c3C)c2[nH]1] 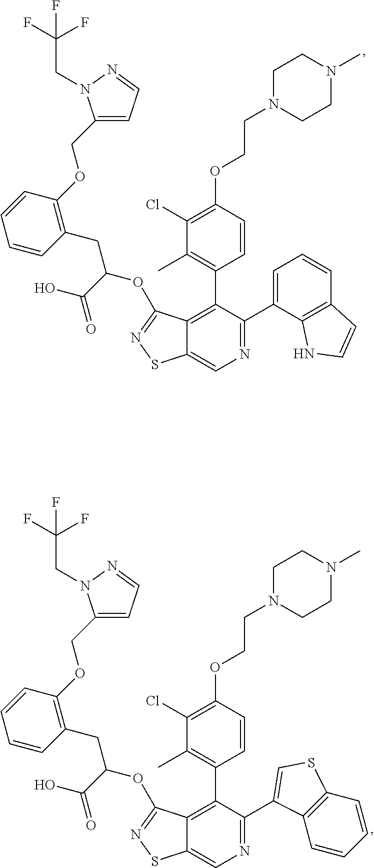 [SMILES: Cc1c(-c2c(-c3cccc4cc[nH]c34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3csc4ccccc34)ncc3snc(OC(Cc4ccccc4OCc4ccnn4CC(F)(F)F)C(=O)O)c23)ccc(OCCN2CCN(C)CC2)c1Cl] 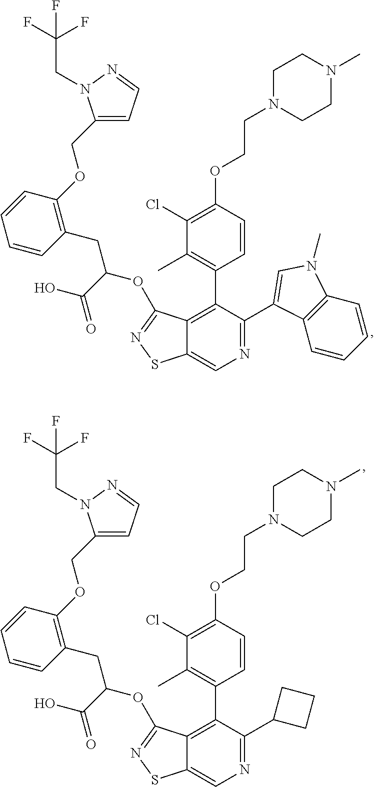 [SMILES: Cc1c(-c2c(-c3cn(C)c4ccccc34)ncc3snc(OC(Cc4ccccc4OCc4ccnn4CC(F)(F)F)C(=O)O)c23)ccc(OCCN2CCN(C)CC2)c1Cl ; Cc1c(-c2c(C3CCC3)ncc3snc(OC(Cc4ccccc4OCc4ccnn4CC(F)(F)F)C(=O)O)c23)ccc(OCCN2CCN(C)CC2)c1Cl] 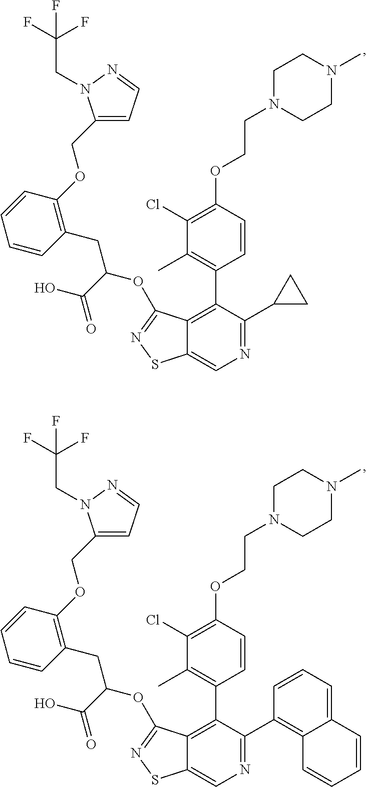 [SMILES: Cc1c(-c2c(C3CC3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccccc34)ncc3snc(OC(Cc4ccccc4OCc4ccnn4CC(F)(F)F)C(=O)O)c23)ccc(OCCN2CCN(C)CC2)c1Cl] 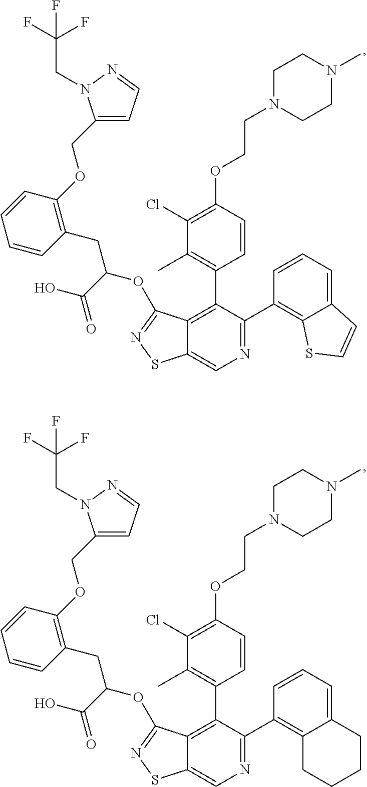 [SMILES: Cc1c(-c2c(-c3cccc4ccsc34)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4c3CCCC4)ncc3snc(OC(Cc4ccccc4OCc4ccnn4CC(F)(F)F)C(=O)O)c23)ccc(OCCN2CCN(C)CC2)c1Cl] 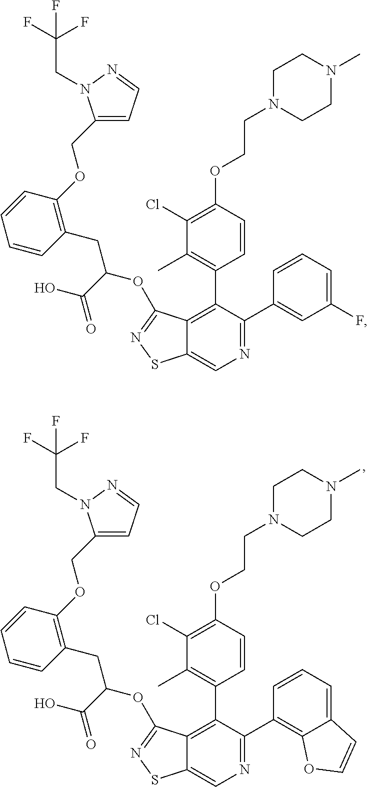 [SMILES: Cc1c(-c2c(-c3cccc(F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc4ccoc34)ncc3snc(OC(Cc4ccccc4OCc4ccnn4CC(F)(F)F)C(=O)O)c23)ccc(OCCN2CCN(C)CC2)c1Cl] 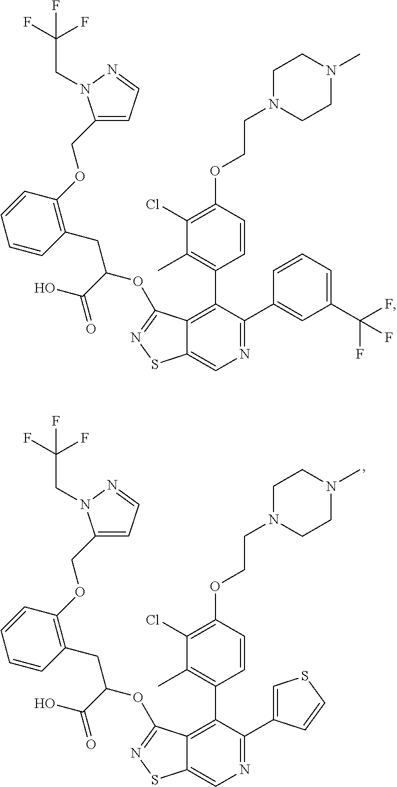 [SMILES: Cc1c(-c2c(-c3cccc(C(F)(F)F)c3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3ccsc3)ncc3snc(OC(Cc4ccccc4OCc4ccnn4CC(F)(F)F)C(=O)O)c23)ccc(OCCN2CCN(C)CC2)c1Cl] 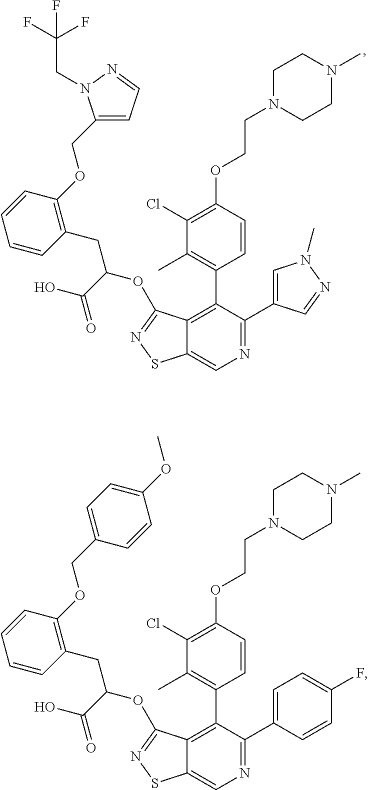 [SMILES: Cc1c(-c2c(-c3cnn(C)c3)ncc3snc(OC(Cc4ccccc4OCc4ccnn4CC(F)(F)F)C(=O)O)c23)ccc(OCCN2CCN(C)CC2)c1Cl ; COc1ccc(COc2ccccc2CC(Oc2nsc3cnc(-c4ccc(F)cc4)c(-c4ccc(OCCN5CCN(C)CC5)c(Cl)c4C)c23)C(=O)O)cc1] 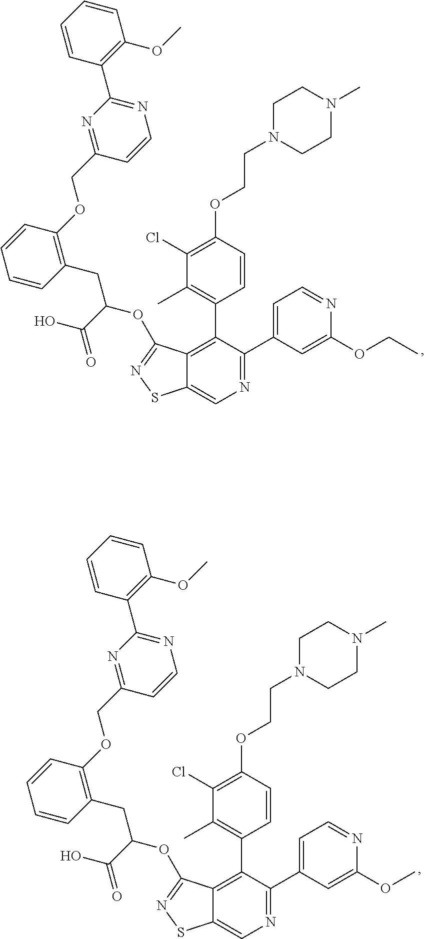 [SMILES: CCOc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccn1] 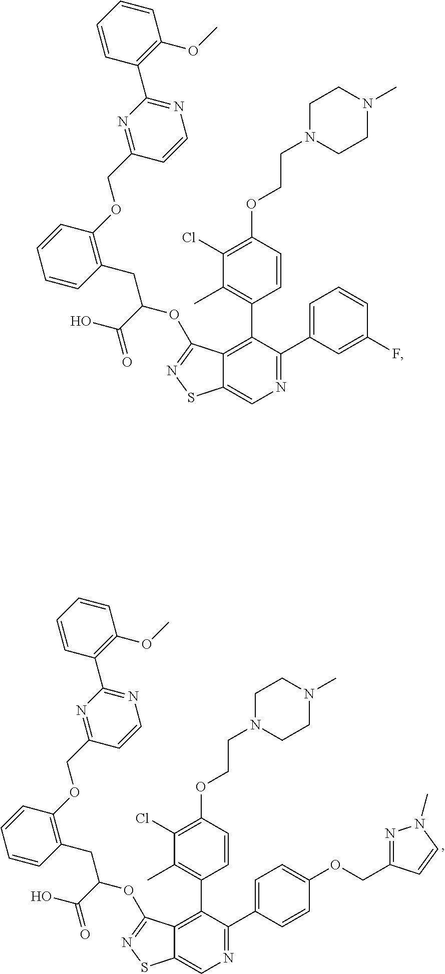 [SMILES: COc1ccccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(OCc5ccn(C)n5)cc4)c(-c4ccc(OCCN5CCN(C)CC5)c(Cl)c4C)c23)C(=O)O)n1] 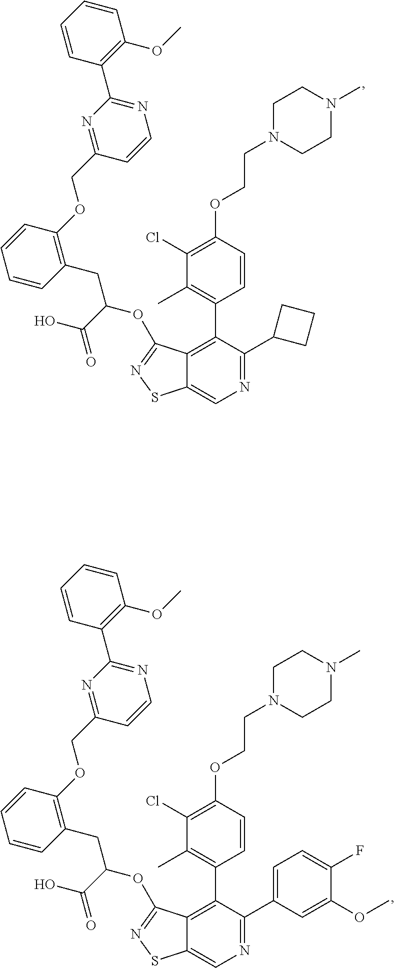 [SMILES: COc1ccccc1-c1nccc(COc2ccccc2CC(Oc2nsc3cnc(C4CCC4)c(-c4ccc(OCCN5CCN(C)CC5)c(Cl)c4C)c23)C(=O)O)n1 ; COc1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)ccc1F] 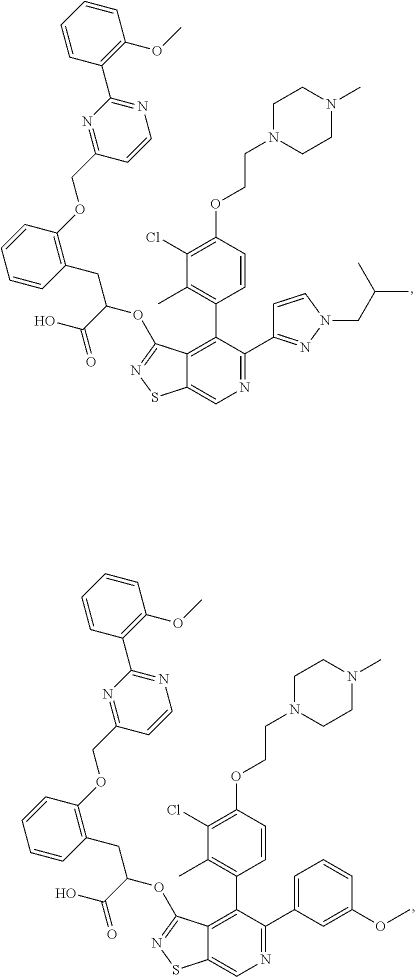 [SMILES: COc1ccccc1-c1nccc(COc2ccccc2CC(Oc2nsc3cnc(-c4ccn(CC(C)C)n4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1] 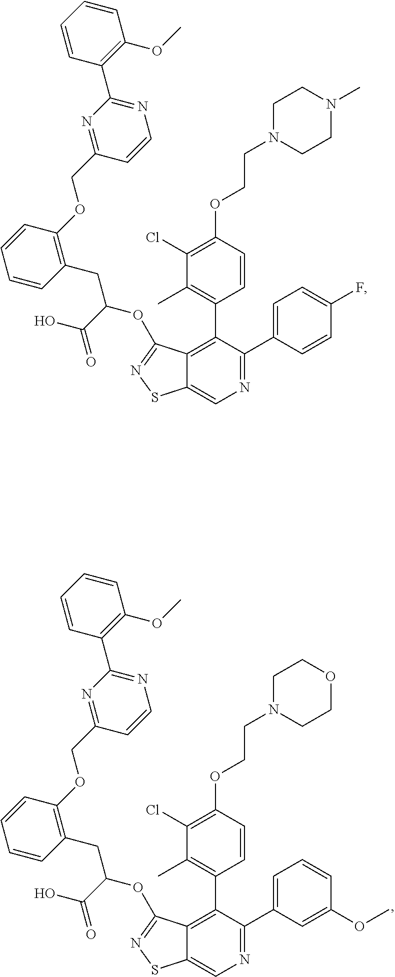 [SMILES: COc1ccccc1-c1nccc(COc2ccccc2CC(Oc2nsc3cnc(-c4ccc(F)cc4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC)n4)C(=O)O)c3c2-c2ccc(OCCN3CCOCC3)c(Cl)c2C)c1] 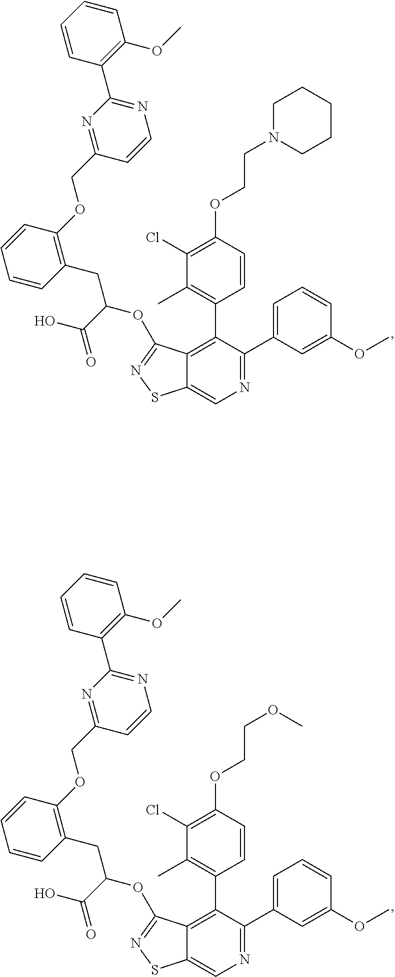 [SMILES: COc1cccc(-c2ncc3snc(OC(Cc4ccccc4OCc4ccnc(-c5ccccc5OC)n4)C(=O)O)c3c2-c2ccc(OCCN3CCCCC3)c(Cl)c2C)c1 ; COCCOc1ccc(-c2c(-c3cccc(OC)c3)ncc3snc(OC(Cc4ccccc4OCc4ccnc(-c5ccccc5OC)n4)C(=O)O)c23)c(C)c1Cl] 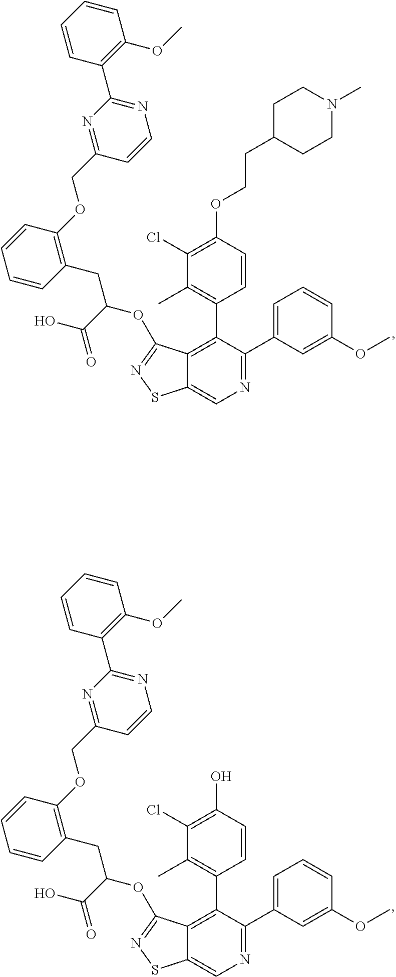 [SMILES: COc1cccc(-c2ncc3snc(OC(Cc4ccccc4OCc4ccnc(-c5ccccc5OC)n4)C(=O)O)c3c2-c2ccc(OCCC3CCN(C)CC3)c(Cl)c2C)c1 ; COc1cccc(-c2ncc3snc(OC(Cc4ccccc4OCc4ccnc(-c5ccccc5OC)n4)C(=O)O)c3c2-c2ccc(O)c(Cl)c2C)c1] 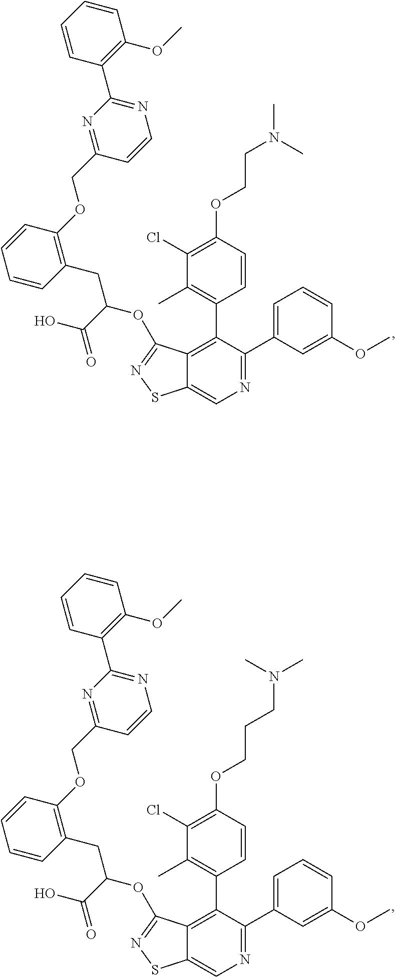 [SMILES: COc1cccc(-c2ncc3snc(OC(Cc4ccccc4OCc4ccnc(-c5ccccc5OC)n4)C(=O)O)c3c2-c2ccc(OCCN(C)C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCCN(C)C)c(Cl)c2C)c1] 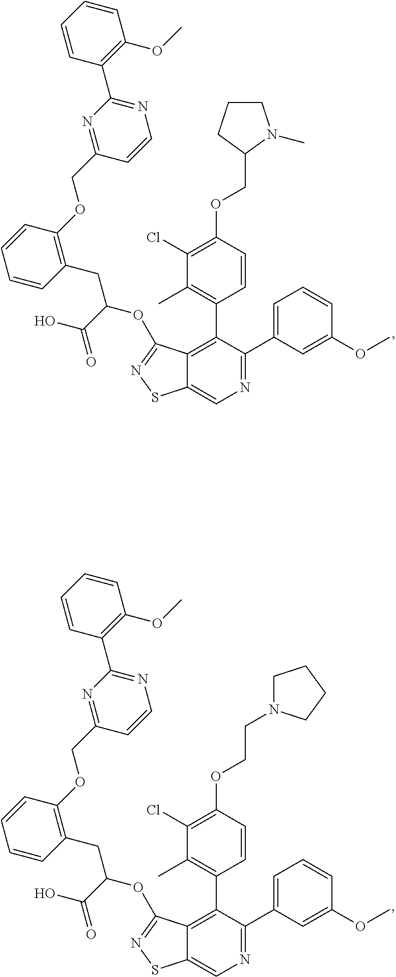 [SMILES: COc1cccc(-c2ncc3snc(OC(Cc4ccccc4OCc4ccnc(-c5ccccc5OC)n4)C(=O)O)c3c2-c2ccc(OCC3CCCN3C)c(Cl)c2C)c1 ; COc1cccc(-c2ncc3snc(OC(Cc4ccccc4OCc4ccnc(-c5ccccc5OC)n4)C(=O)O)c3c2-c2ccc(OCCN3CCCC3)c(Cl)c2C)c1] 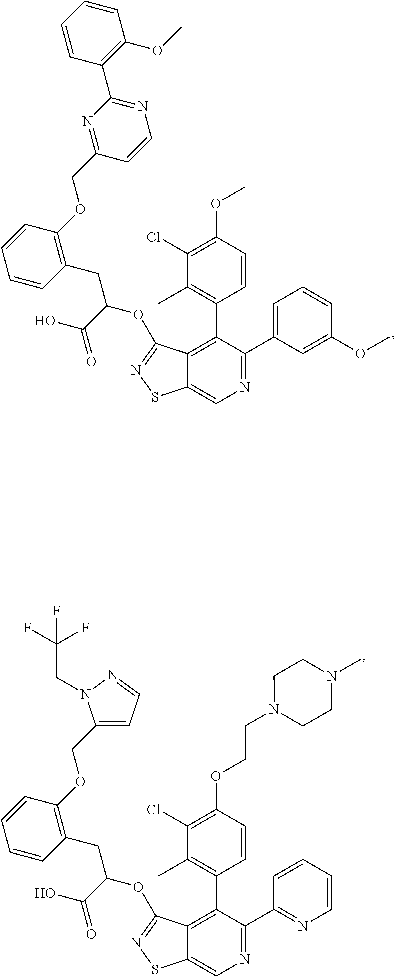 [SMILES: COc1cccc(-c2ncc3snc(OC(Cc4ccccc4OCc4ccnc(-c5ccccc5OC)n4)C(=O)O)c3c2-c2ccc(OC)c(Cl)c2C)c1 ; Cc1c(-c2c(-c3ccccn3)ncc3snc(OC(Cc4ccccc4OCc4ccnn4CC(F)(F)F)C(=O)O)c23)ccc(OCCN2CCN(C)CC2)c1Cl] 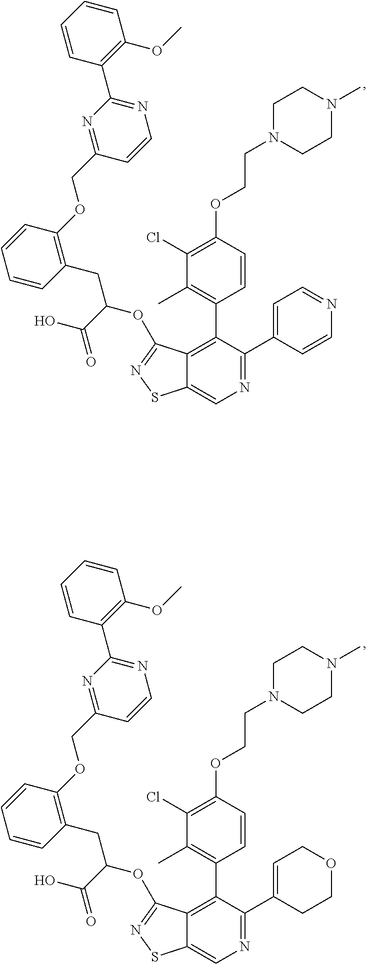 [SMILES: COc1ccccc1-c1nccc(COc2ccccc2CC(Oc2nsc3cnc(-c4ccncc4)c(-c4ccc(OCCN5CCN(C)CC5)c(Cl)c4C)c23)C(=O)O)n1 ; COc1ccccc1-c1nccc(COc2ccccc2CC(Oc2nsc3cnc(C4=CCOCC4)c(-c4ccc(OCCN5CCN(C)CC5)c(Cl)c4C)c23)C(=O)O)n1] 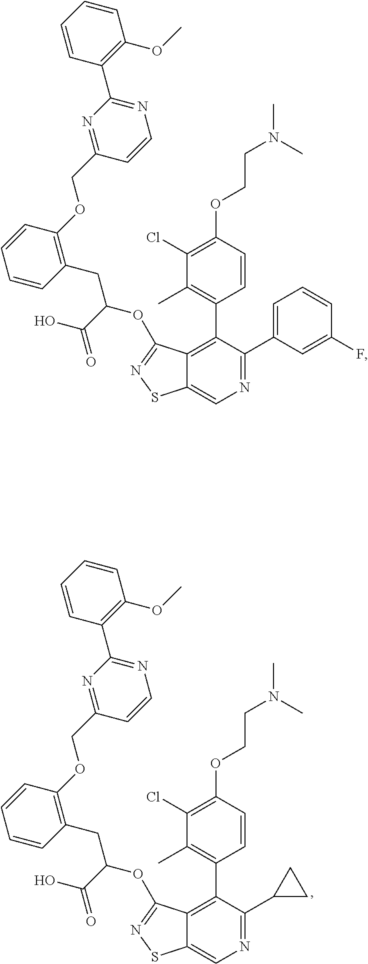 [SMILES: COc1ccccc1-c1nccc(COc2ccccc2CC(Oc2nsc3cnc(-c4cccc(F)c4)c(-c4ccc(OCCN(C)C)c(Cl)c4C)c23)C(=O)O)n1 ; COc1ccccc1-c1nccc(COc2ccccc2CC(Oc2nsc3cnc(C4CC4)c(-c4ccc(OCCN(C)C)c(Cl)c4C)c23)C(=O)O)n1] 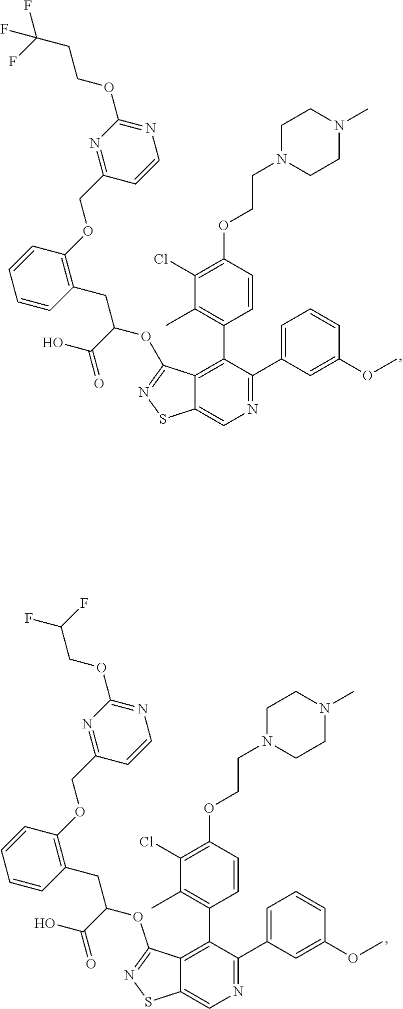 [SMILES: COc1cccc(-c2ncc3snc(OC(Cc4ccccc4OCc4ccnc(OCCC(F)(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(OCC(F)F)n4)C(=O)O)c3c2-c2ccc(OCCN3CCN(C)CC3)c(Cl)c2C)c1] 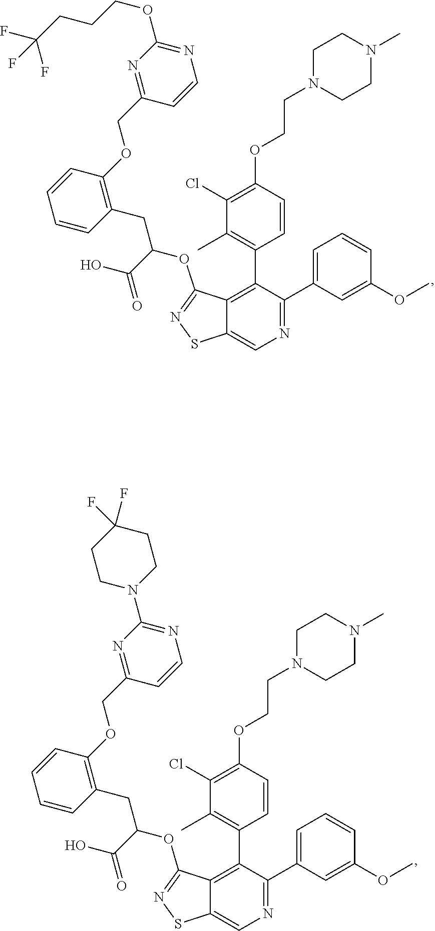 [SMILES: COc1cccc(-c2ncc3snc(OC(Cc4ccccc4OCc4ccnc(OCCCC(F)(F)F)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCC(F)(F)CC5)n4)C(=O)O)c3c2-c2ccc(OCCN3CCN(C)CC3)c(Cl)c2C)c1] 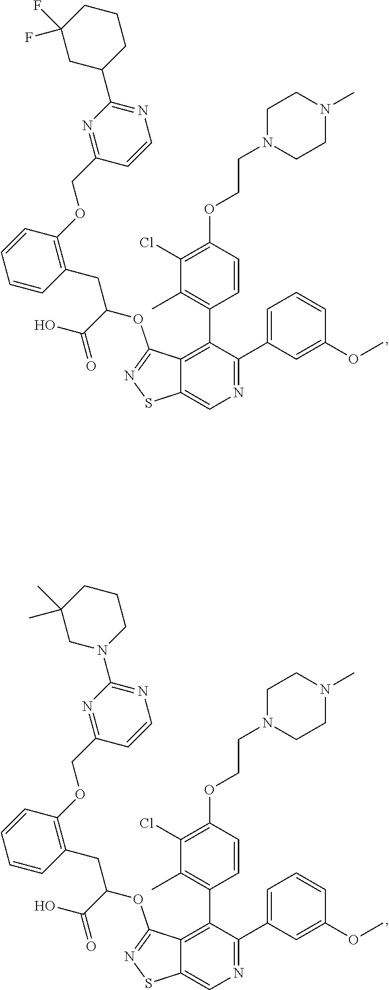 [SMILES: COc1cccc(-c2ncc3snc(OC(Cc4ccccc4OCc4ccnc(C5CCCC(F)(F)C5)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(N5CCCC(C)(C)C5)n4)C(=O)O)c3c2-c2ccc(OCCN3CCN(C)CC3)c(Cl)c2C)c1] 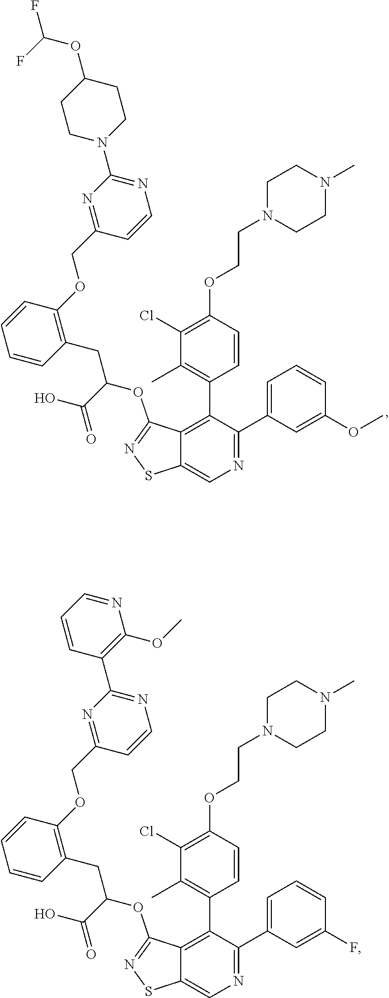 [SMILES: COc1cccc(-c2ncc3snc(OC(Cc4ccccc4OCc4ccnc(N5CCC(OC(F)F)CC5)n4)C(=O)O)c3c2-c2ccc(OCCN3CCN(C)CC3)c(Cl)c2C)c1 ; COc1ncccc1-c1nccc(COc2ccccc2CC(Oc2nsc3cnc(-c4cccc(F)c4)c(-c4ccc(OCCN5CCN(C)CC5)c(Cl)c4C)c23)C(=O)O)n1] 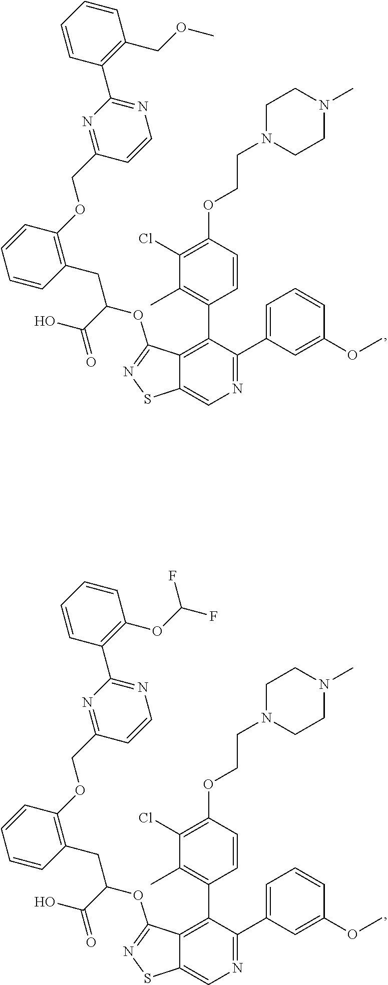 [SMILES: COCc1ccccc1-c1nccc(COc2ccccc2CC(Oc2nsc3cnc(-c4cccc(OC)c4)c(-c4ccc(OCCN5CCN(C)CC5)c(Cl)c4C)c23)C(=O)O)n1 ; COc1cccc(-c2ncc3snc(OC(Cc4ccccc4OCc4ccnc(-c5ccccc5OC(F)F)n4)C(=O)O)c3c2-c2ccc(OCCN3CCN(C)CC3)c(Cl)c2C)c1] 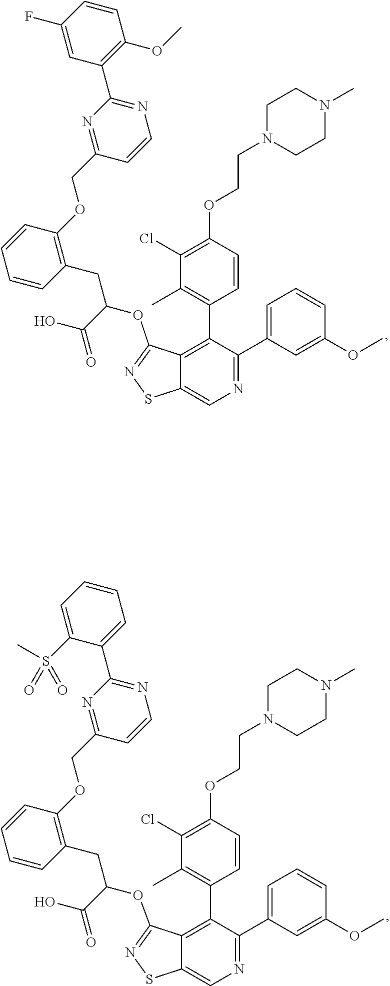 [SMILES: COc1cccc(-c2ncc3snc(OC(Cc4ccccc4OCc4ccnc(-c5cc(F)ccc5OC)n4)C(=O)O)c3c2-c2ccc(OCCN3CCN(C)CC3)c(Cl)c2C)c1 ; COc1cccc(-c2ncc3snc(OC(Cc4ccccc4OCc4ccnc(-c5ccccc5S(C)(=O)=O)n4)C(=O)O)c3c2-c2ccc(OCCN3CCN(C)CC3)c(Cl)c2C)c1] 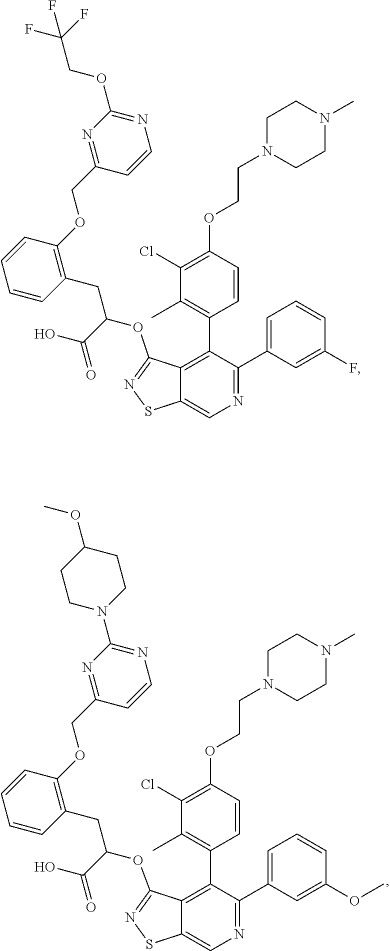 [SMILES: Cc1c(-c2c(-c3cccc(F)c3)ncc3snc(OC(Cc4ccccc4OCc4ccnc(OCC(F)(F)F)n4)C(=O)O)c23)ccc(OCCN2CCN(C)CC2)c1Cl ; COc1cccc(-c2ncc3snc(OC(Cc4ccccc4OCc4ccnc(N5CCC(OC)CC5)n4)C(=O)O)c3c2-c2ccc(OCCN3CCN(C)CC3)c(Cl)c2C)c1] 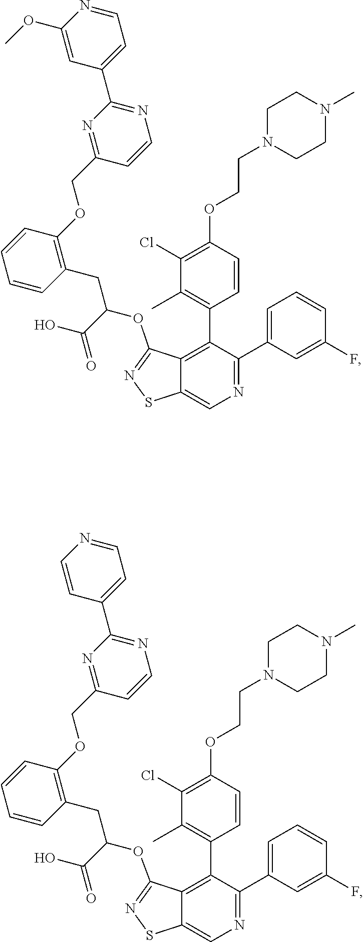 [SMILES: COc1cc(-c2nccc(COc3ccccc3CC(Oc3nsc4cnc(-c5cccc(F)c5)c(-c5ccc(OCCN6CCN(C)CC6)c(Cl)c5C)c34)C(=O)O)n2)ccn1 ; Cc1c(-c2c(-c3cccc(F)c3)ncc3snc(OC(Cc4ccccc4OCc4ccnc(-c5ccncc5)n4)C(=O)O)c23)ccc(OCCN2CCN(C)CC2)c1Cl] 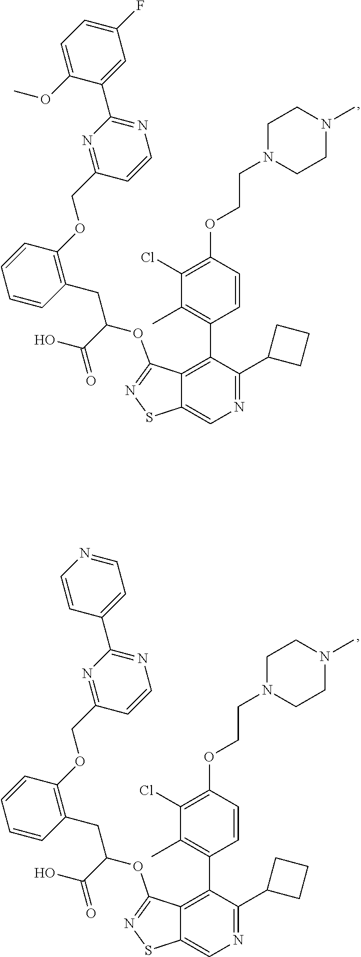 [SMILES: COc1ccc(F)cc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5ccncc5)n4)C(=O)O)c23)ccc(OCCN2CCN(C)CC2)c1Cl] 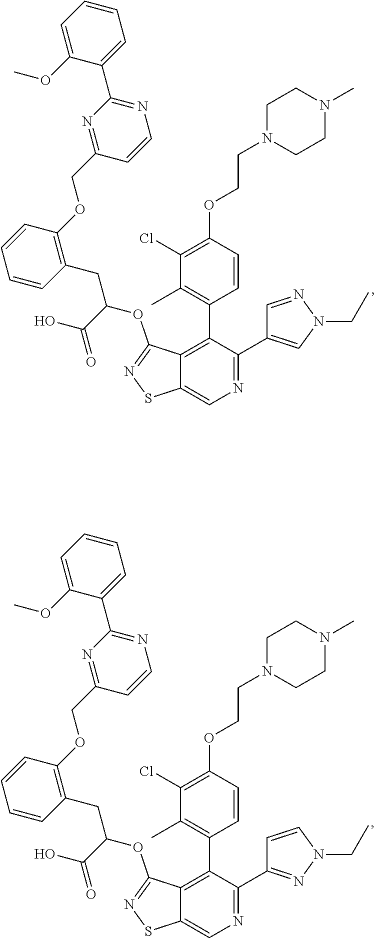 [SMILES: CCn1cc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)cn1 ; CCn1ccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)n1] 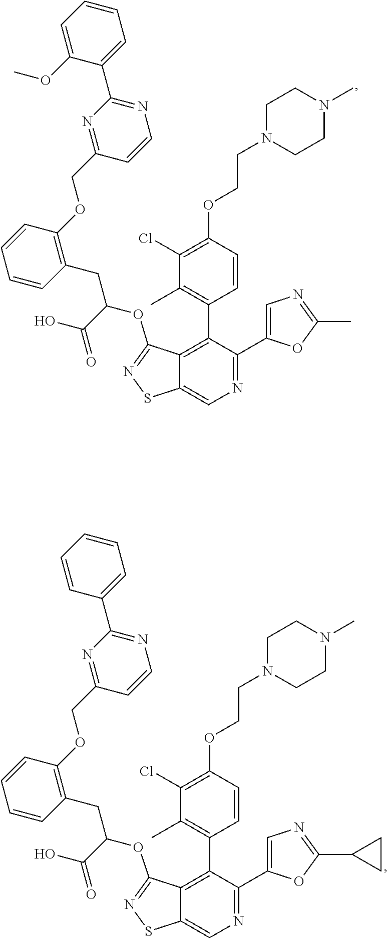 [SMILES: COc1ccccc1-c1nccc(COc2ccccc2CC(Oc2nsc3cnc(-c4cnc(C)o4)c(-c4ccc(OCCN5CCN(C)CC5)c(Cl)c4C)c23)C(=O)O)n1 ; Cc1c(-c2c(-c3cnc(C4CC4)o3)ncc3snc(OC(Cc4ccccc4OCc4ccnc(-c5ccccc5)n4)C(=O)O)c23)ccc(OCCN2CCN(C)CC2)c1Cl] 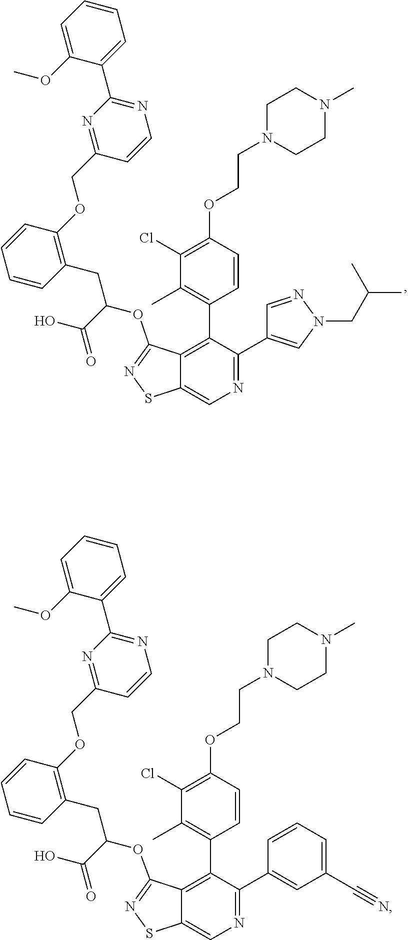 [SMILES: COc1ccccc1-c1nccc(COc2ccccc2CC(Oc2nsc3cnc(-c4cnn(CC(C)C)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cccc(C#N)c4)c(-c4ccc(OCCN5CCN(C)CC5)c(Cl)c4C)c23)C(=O)O)n1] 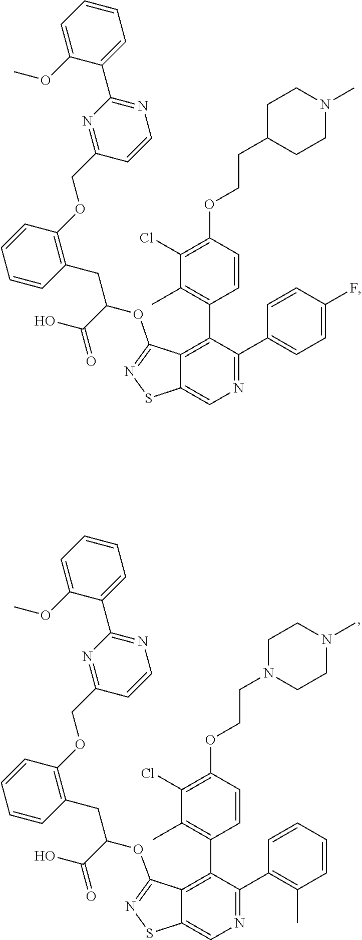 [SMILES: COc1ccccc1-c1nccc(COc2ccccc2CC(Oc2nsc3cnc(-c4ccc(F)cc4)c(-c4ccc(OCCC5CCN(C)CC5)c(Cl)c4C)c23)C(=O)O)n1 ; COc1ccccc1-c1nccc(COc2ccccc2CC(Oc2nsc3cnc(-c4ccccc4C)c(-c4ccc(OCCN5CCN(C)CC5)c(Cl)c4C)c23)C(=O)O)n1] 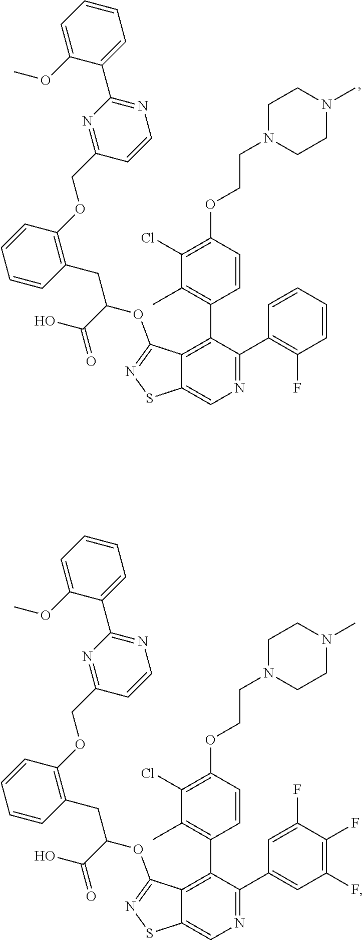 [SMILES: COc1ccccc1-c1nccc(COc2ccccc2CC(Oc2nsc3cnc(-c4ccccc4F)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cc(F)c(F)c(F)c4)c(-c4ccc(OCCN5CCN(C)CC5)c(Cl)c4C)c23)C(=O)O)n1] 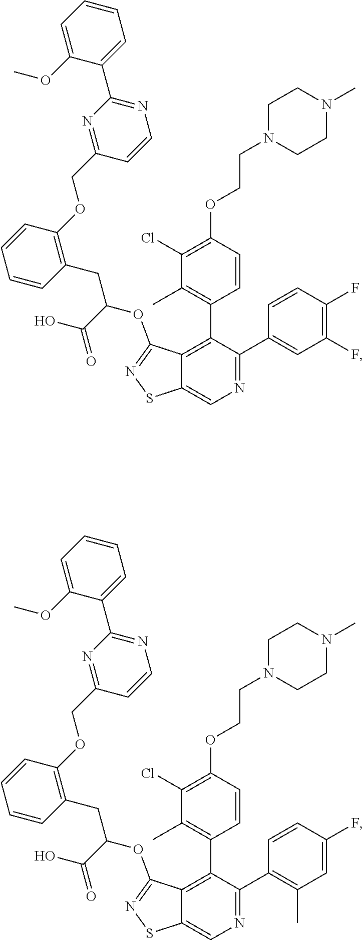 [SMILES: COc1ccccc1-c1nccc(COc2ccccc2CC(Oc2nsc3cnc(-c4ccc(F)c(F)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4ccc(F)cc4C)c(-c4ccc(OCCN5CCN(C)CC5)c(Cl)c4C)c23)C(=O)O)n1] 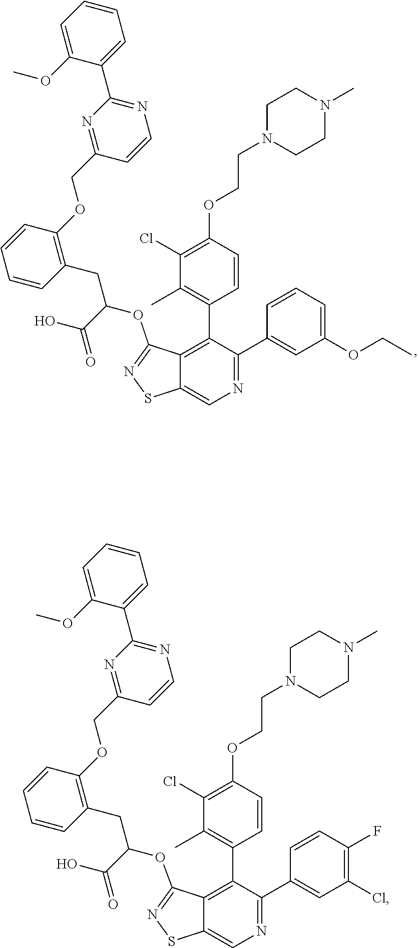 [SMILES: CCOc1cccc(-c2ncc3snc(OC(Cc4ccccc4OCc4ccnc(-c5ccccc5OC)n4)C(=O)O)c3c2-c2ccc(OCCN3CCN(C)CC3)c(Cl)c2C)c1 ; COc1ccccc1-c1nccc(COc2ccccc2CC(Oc2nsc3cnc(-c4ccc(F)c(Cl)c4)c(-c4ccc(OCCN5CCN(C)CC5)c(Cl)c4C)c23)C(=O)O)n1] 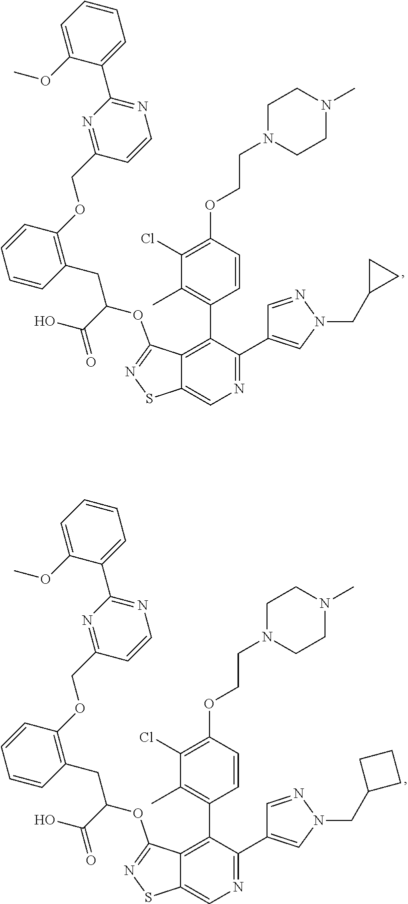 [SMILES: COc1ccccc1-c1nccc(COc2ccccc2CC(Oc2nsc3cnc(-c4cnn(CC5CC5)c4)c(-c4ccc(OCCN5CCN(C)CC5)c(Cl)c4C)c23)C(=O)O)n1 ; COc1ccccc1-c1nccc(COc2ccccc2CC(Oc2nsc3cnc(-c4cnn(CC5CCC5)c4)c(-c4ccc(OCCN5CCN(C)CC5)c(Cl)c4C)c23)C(=O)O)n1] 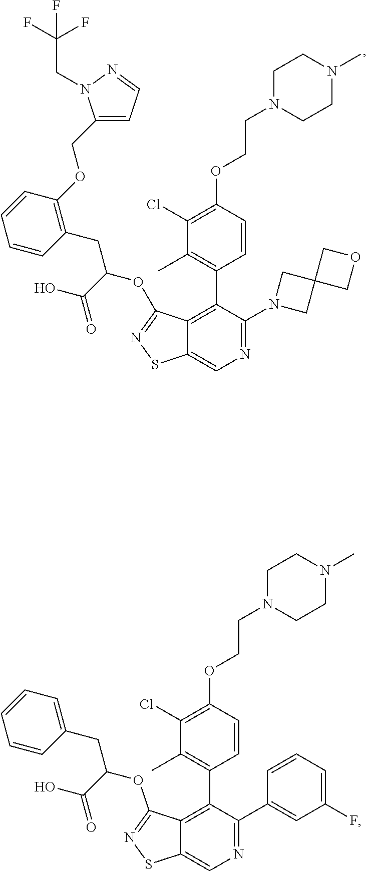 [SMILES: Cc1c(-c2c(N3CC4(COC4)C3)ncc3snc(OC(Cc4ccccc4OCc4ccnn4CC(F)(F)F)C(=O)O)c23)ccc(OCCN2CCN(C)CC2)c1Cl ; Cc1c(-c2c(-c3cccc(F)c3)ncc3snc(OC(Cc4ccccc4)C(=O)O)c23)ccc(OCCN2CCN(C)CC2)c1Cl] 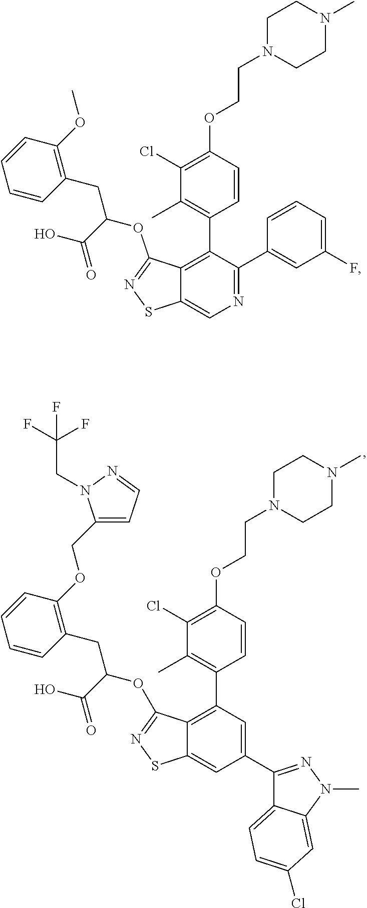 [SMILES: COc1ccccc1CC(Oc1nsc2cnc(-c3cccc(F)c3)c(-c3ccc(OCCN4CCN(C)CC4)c(Cl)c3C)c12)C(=O)O ; Cc1c(-c2cc(-c3nn(C)c4cc(Cl)ccc34)cc3snc(OC(Cc4ccccc4OCc4ccnn4CC(F)(F)F)C(=O)O)c23)ccc(OCCN2CCN(C)CC2)c1Cl] 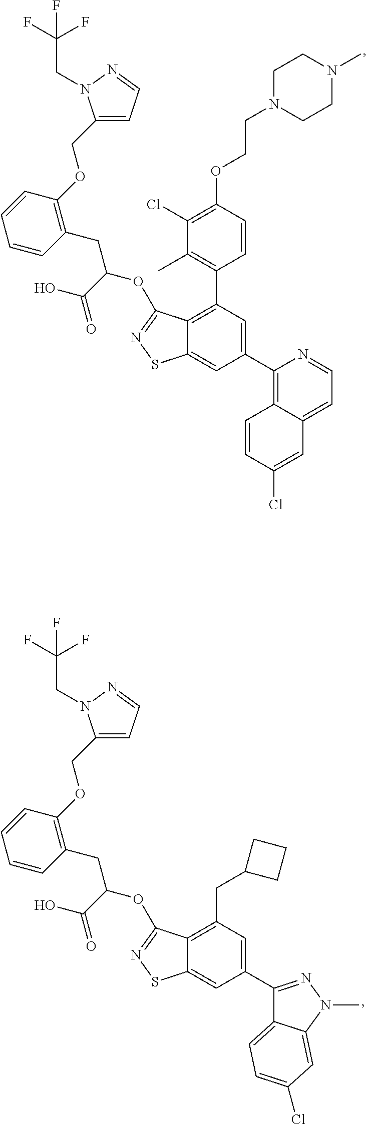 [SMILES: Cc1c(-c2cc(-c3nccc4cc(Cl)ccc34)cc3snc(OC(Cc4ccccc4OCc4ccnn4CC(F)(F)F)C(=O)O)c23)ccc(OCCN2CCN(C)CC2)c1Cl ; Cn1nc(-c2cc(CC3CCC3)c3c(OC(Cc4ccccc4OCc4ccnn4CC(F)(F)F)C(=O)O)nsc3c2)c2ccc(Cl)cc21] 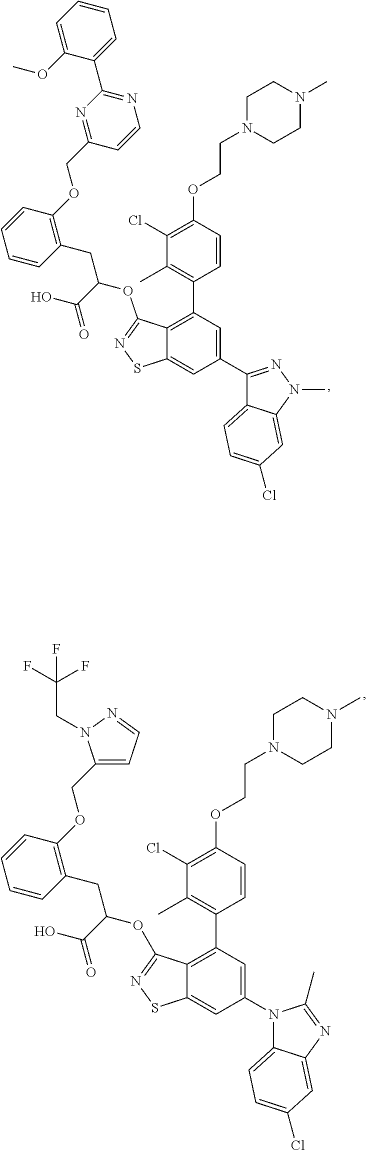 [SMILES: COc1ccccc1-c1nccc(COc2ccccc2CC(Oc2nsc3cc(-c4nn(C)c5cc(Cl)ccc45)cc(-c4ccc(OCCN5CCN(C)CC5)c(Cl)c4C)c23)C(=O)O)n1 ; Cc1c(-c2cc(-n3c(C)nc4cc(Cl)ccc43)cc3snc(OC(Cc4ccccc4OCc4ccnn4CC(F)(F)F)C(=O)O)c23)ccc(OCCN2CCN(C)CC2)c1Cl] 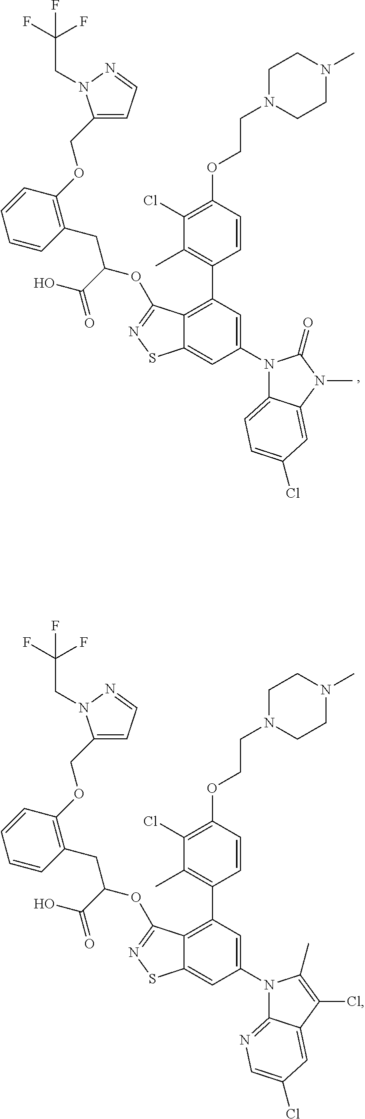 [SMILES: Cc1c(-c2cc(-n3c(=O)n(C)c4cc(Cl)ccc43)cc3snc(OC(Cc4ccccc4OCc4ccnn4CC(F)(F)F)C(=O)O)c23)ccc(OCCN2CCN(C)CC2)c1Cl ; Cc1c(-c2cc(-n3c(C)c(Cl)c4cc(Cl)cnc43)cc3snc(OC(Cc4ccccc4OCc4ccnn4CC(F)(F)F)C(=O)O)c23)ccc(OCCN2CCN(C)CC2)c1Cl] 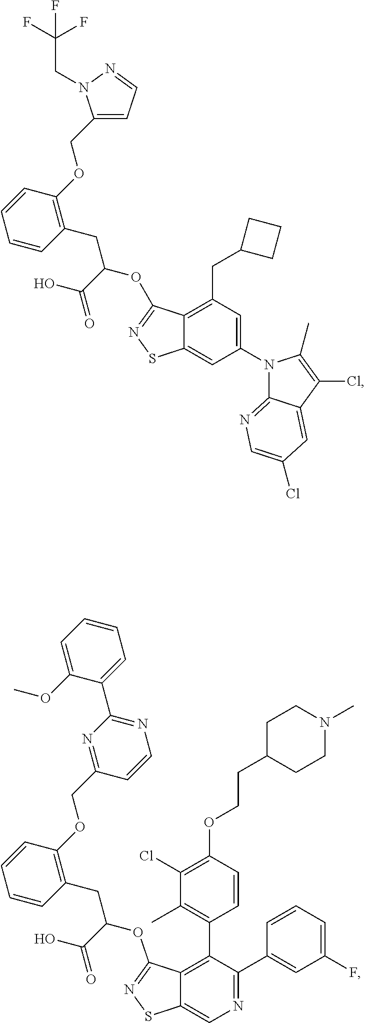 [SMILES: Cc1c(Cl)c2cc(Cl)cnc2n1-c1cc(CC2CCC2)c2c(OC(Cc3ccccc3OCc3ccnn3CC(F)(F)F)C(=O)O)nsc2c1 ; COc1ccccc1-c1nccc(COc2ccccc2CC(Oc2nsc3cnc(-c4cccc(F)c4)c(-c4ccc(OCCC5CCN(C)CC5)c(Cl)c4C)c23)C(=O)O)n1] 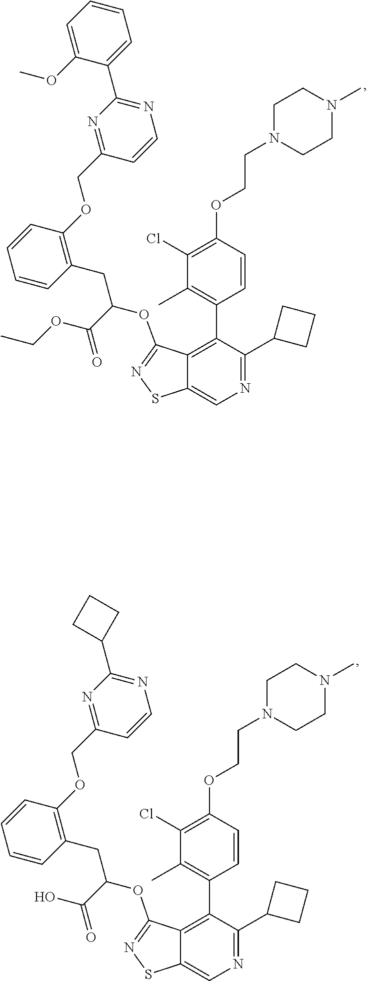 [SMILES: CCOC(=O)C(Cc1ccccc1OCc1ccnc(-c2ccccc2OC)n1)Oc1nsc2cnc(C3CCC3)c(-c3ccc(OCCN4CCN(C)CC4)c(Cl)c3C)c12 ; Cc1c(-c2c(C3CCC3)ncc3snc(OC(Cc4ccccc4OCc4ccnc(C5CCC5)n4)C(=O)O)c23)ccc(OCCN2CCN(C)CC2)c1Cl] 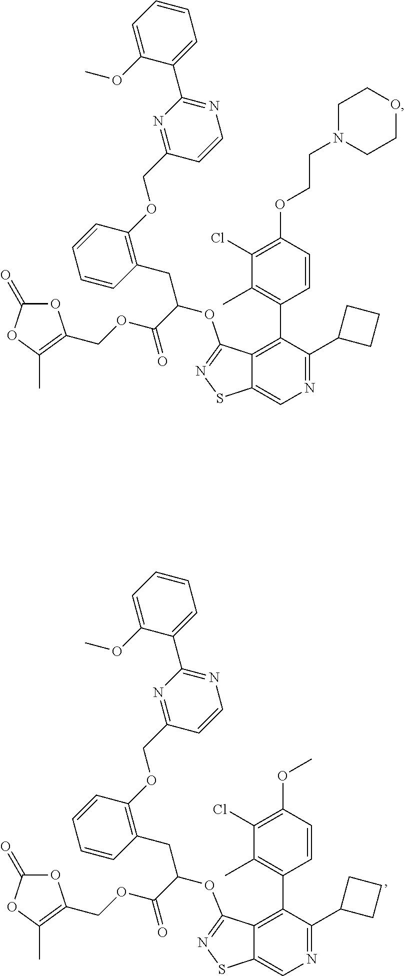 [SMILES: COc1ccccc1-c1nccc(COc2ccccc2CC(Oc2nsc3cnc(C4CCC4)c(-c4ccc(OCCN5CCOCC5)c(Cl)c4C)c23)C(=O)OCc2oc(=O)oc2C)n1 ; COc1ccccc1-c1nccc(COc2ccccc2CC(Oc2nsc3cnc(C4CCC4)c(-c4ccc(OC)c(Cl)c4C)c23)C(=O)OCc2oc(=O)oc2C)n1] 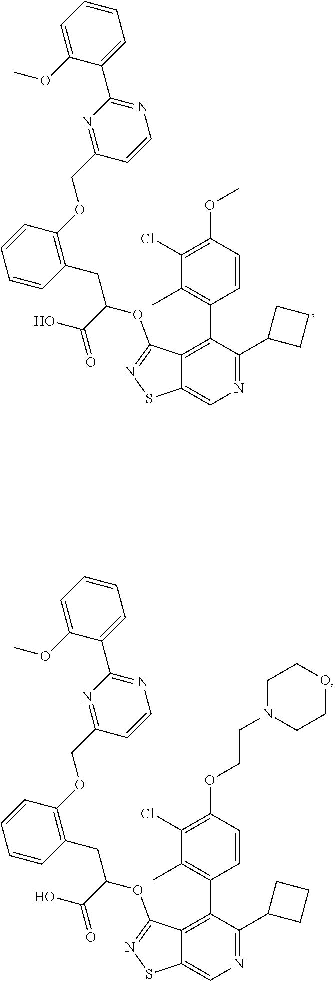 [SMILES: COc1ccccc1-c1nccc(COc2ccccc2CC(Oc2nsc3cnc(C4CCC4)c(-c4ccc(OC)c(Cl)c4C)c23)C(=O)O)n1 ; COc1ccccc1-c1nccc(COc2ccccc2CC(Oc2nsc3cnc(C4CCC4)c(-c4ccc(OCCN5CCOCC5)c(Cl)c4C)c23)C(=O)O)n1] 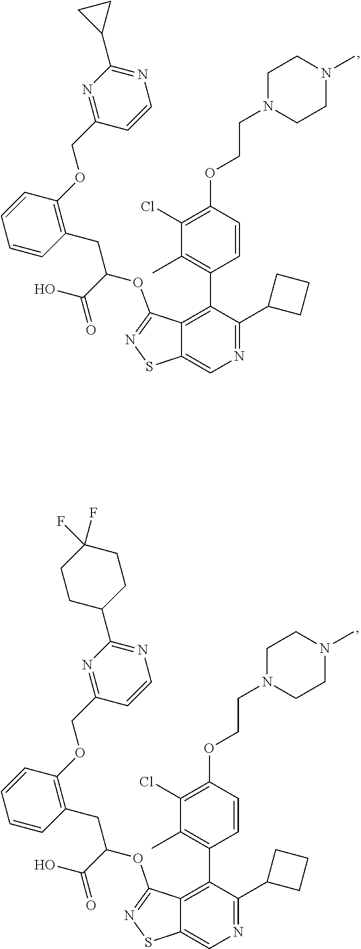 [SMILES: Cc1c(-c2c(C3CCC3)ncc3snc(OC(Cc4ccccc4OCc4ccnc(C5CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCC(F)(F)CC5)n4)C(=O)O)c23)ccc(OCCN2CCN(C)CC2)c1Cl] 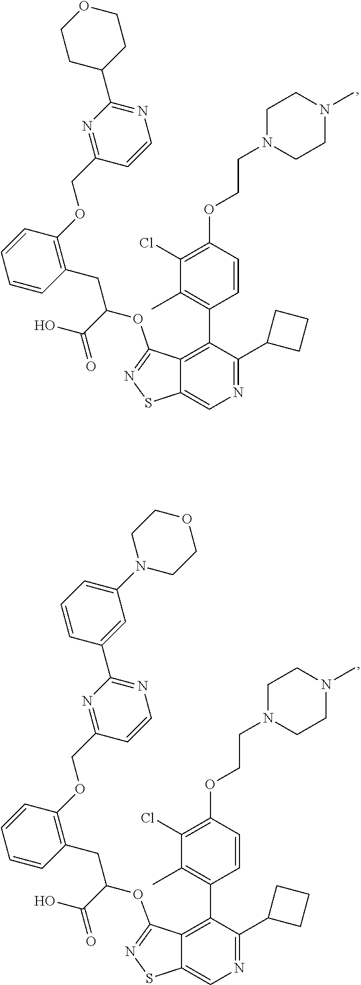 [SMILES: Cc1c(-c2c(C3CCC3)ncc3snc(OC(Cc4ccccc4OCc4ccnc(C5CCOCC5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5cccc(N6CCOCC6)c5)n4)C(=O)O)c23)ccc(OCCN2CCN(C)CC2)c1Cl] 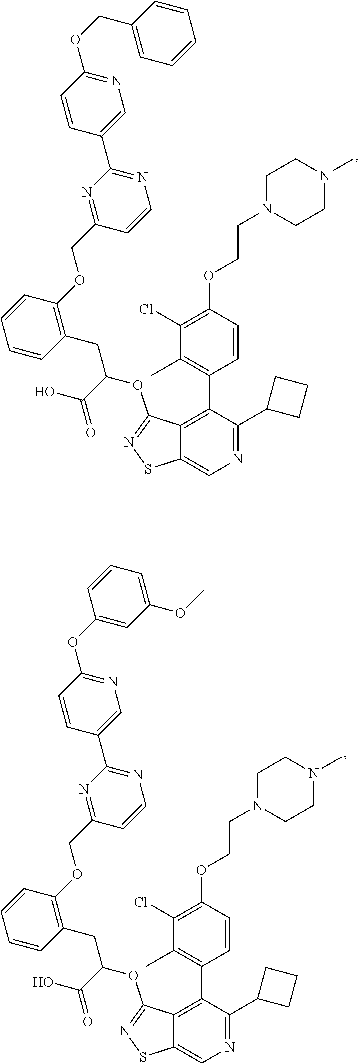 [SMILES: Cc1c(-c2c(C3CCC3)ncc3snc(OC(Cc4ccccc4OCc4ccnc(-c5ccc(OCc6ccccc6)nc5)n4)C(=O)O)c23)ccc(OCCN2CCN(C)CC2)c1Cl ; COc1cccc(Oc2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cn2)c1] 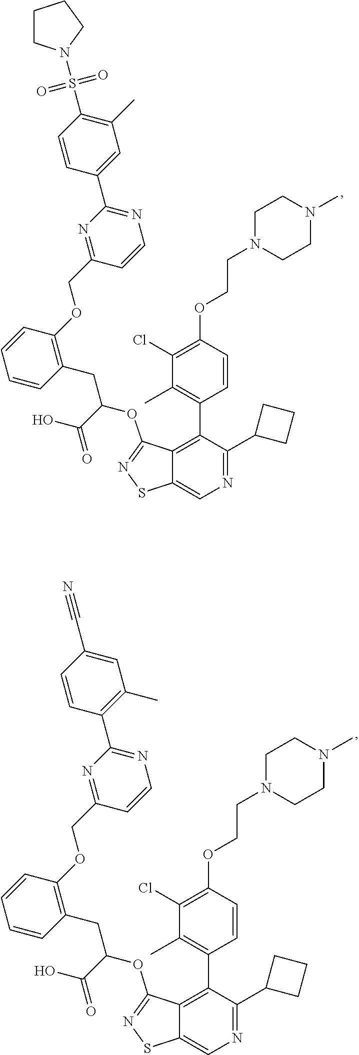 [SMILES: Cc1cc(-c2nccc(COc3ccccc3CC(Oc3nsc4cnc(C5CCC5)c(-c5ccc(OCCN6CCN(C)CC6)c(Cl)c5C)c34)C(=O)O)n2)ccc1S(=O)(=O)N1CCCC1 ; Cc1cc(C#N)ccc1-c1nccc(COc2ccccc2CC(Oc2nsc3cnc(C4CCC4)c(-c4ccc(OCCN5CCN(C)CC5)c(Cl)c4C)c23)C(=O)O)n1] 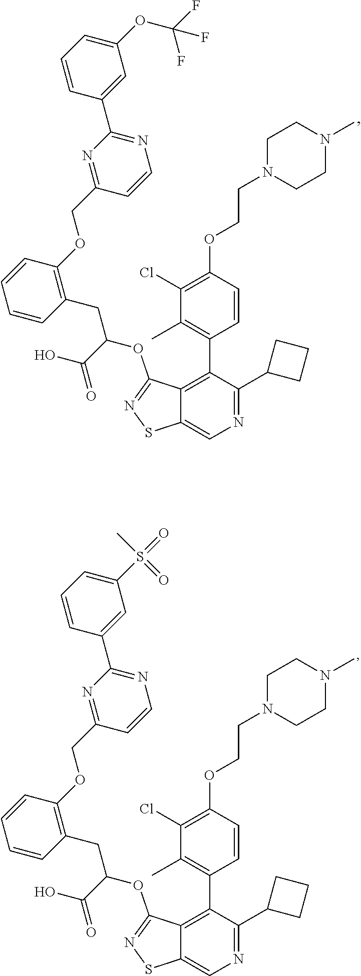 [SMILES: Cc1c(-c2c(C3CCC3)ncc3snc(OC(Cc4ccccc4OCc4ccnc(-c5cccc(OC(F)(F)F)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5cccc(S(C)(=O)=O)c5)n4)C(=O)O)c23)ccc(OCCN2CCN(C)CC2)c1Cl] 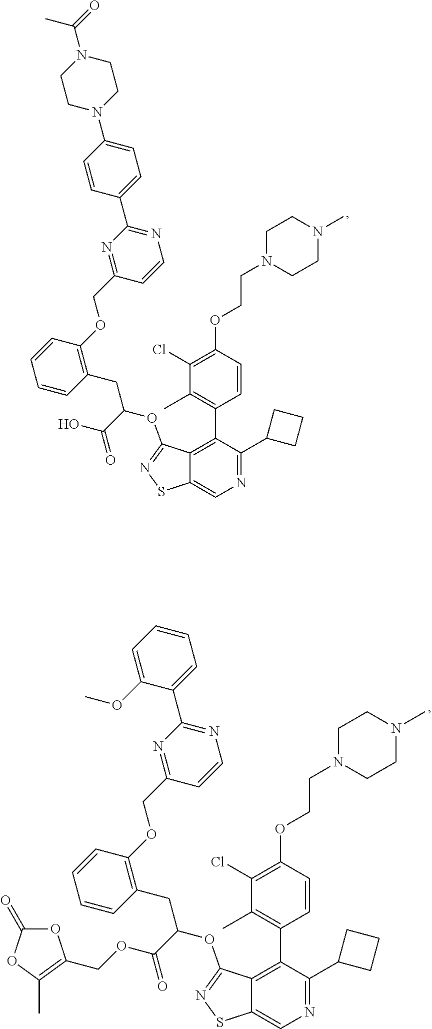 [SMILES: CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cc2)CC1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)OCc2oc(=O)oc2C)n1] 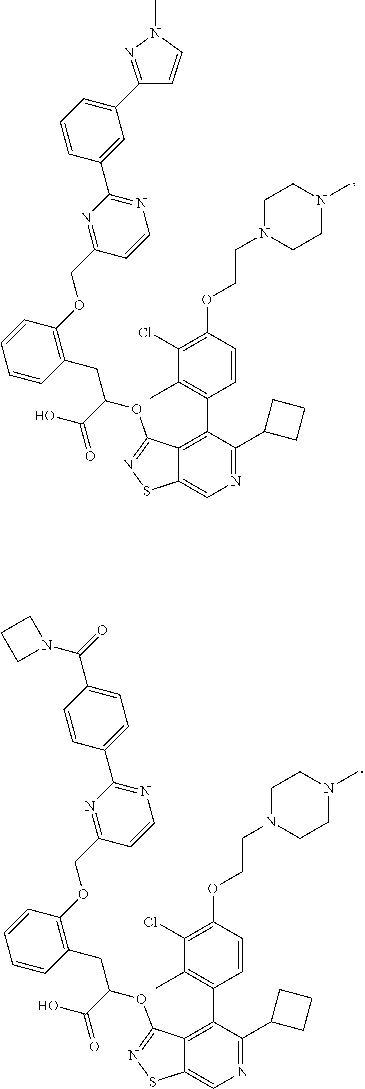 [SMILES: Cc1c(-c2c(C3CCC3)ncc3snc(OC(Cc4ccccc4OCc4ccnc(-c5cccc(-c6ccn(C)n6)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(C(=O)N6CCC6)cc5)n4)C(=O)O)c23)ccc(OCCN2CCN(C)CC2)c1Cl] 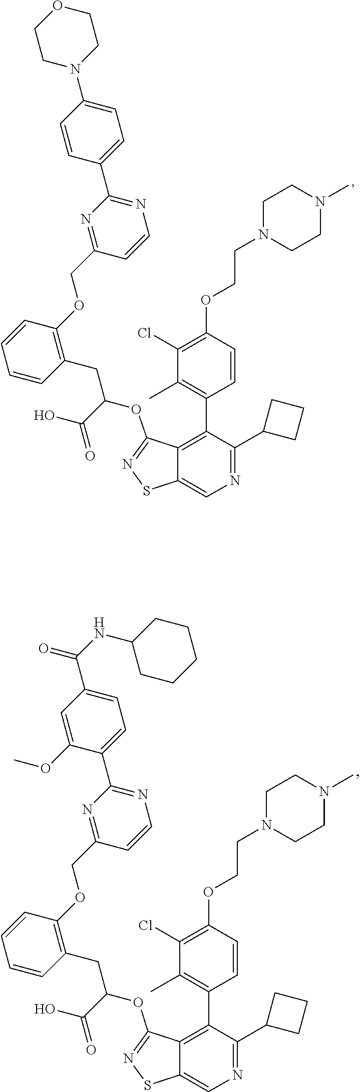 [SMILES: Cc1c(-c2c(C3CCC3)ncc3snc(OC(Cc4ccccc4OCc4ccnc(-c5ccc(N6CCOCC6)cc5)n4)C(=O)O)c23)ccc(OCCN2CCN(C)CC2)c1Cl ; COc1cc(C(=O)NC2CCCCC2)ccc1-c1nccc(COc2ccccc2CC(Oc2nsc3cnc(C4CCC4)c(-c4ccc(OCCN5CCN(C)CC5)c(Cl)c4C)c23)C(=O)O)n1] 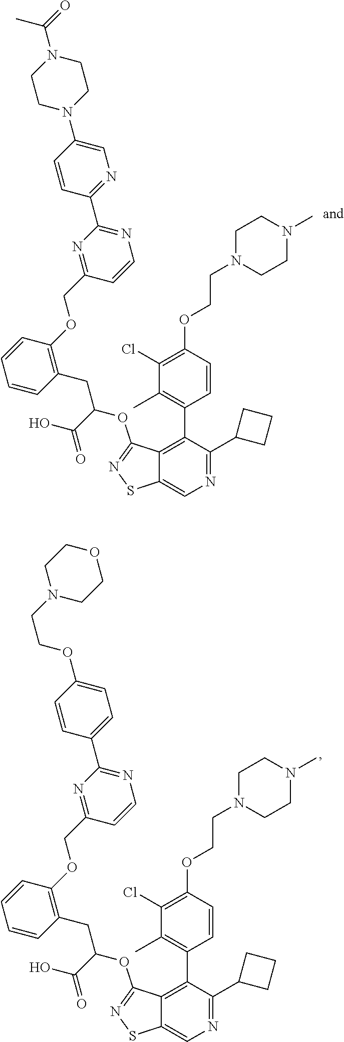 [SMILES: CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)nc2)CC1 ; Cc1c(-c2c(C3CCC3)ncc3snc(OC(Cc4ccccc4OCc4ccnc(-c5ccc(OCCN6CCOCC6)cc5)n4)C(=O)O)c23)ccc(OCCN2CCN(C)CC2)c1Cl]
or a pharmaceutically acceptable salt thereof.

51. A compound selected from: 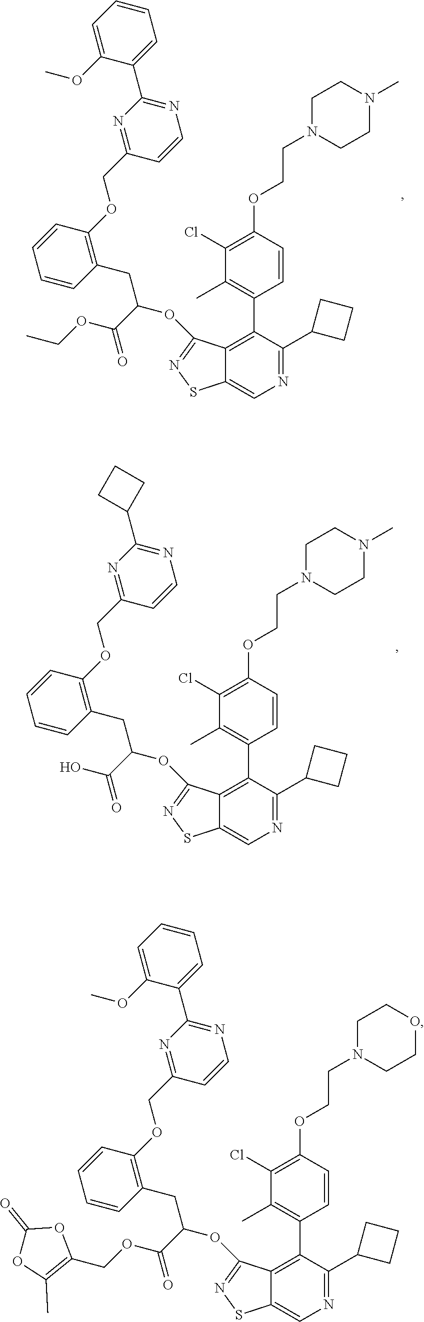 [SMILES: CCOC(=O)C(Cc1ccccc1OCc1ccnc(-c2ccccc2OC)n1)Oc1nsc2cnc(C3CCC3)c(-c3ccc(OCCN4CCN(C)CC4)c(Cl)c3C)c12 ; Cc1c(-c2c(C3CCC3)ncc3snc(OC(Cc4ccccc4OCc4ccnc(C5CCC5)n4)C(=O)O)c23)ccc(OCCN2CCN(C)CC2)c1Cl ; COc1ccccc1-c1nccc(COc2ccccc2CC(Oc2nsc3cnc(C4CCC4)c(-c4ccc(OCCN5CCOCC5)c(Cl)c4C)c23)C(=O)OCc2oc(=O)oc2C)n1] 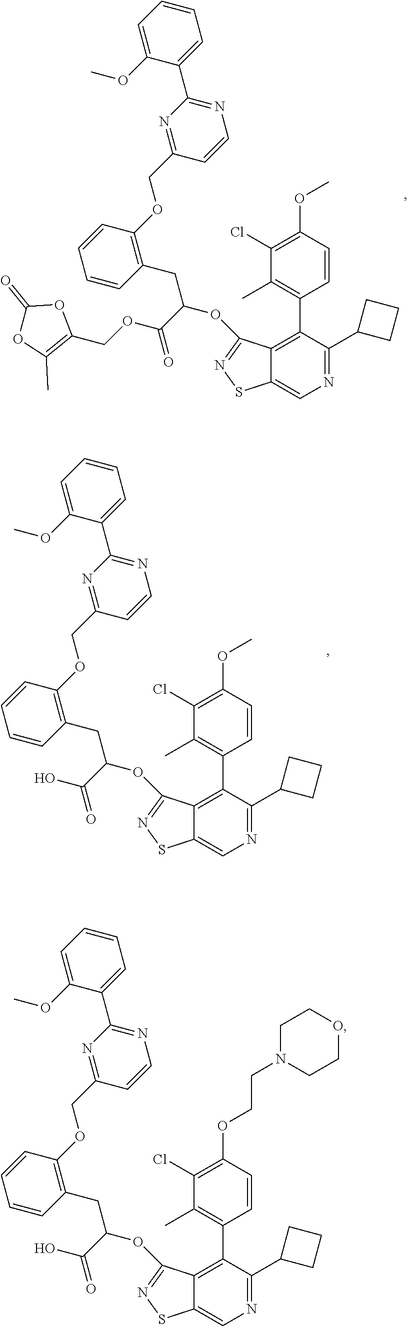 [SMILES: COc1ccccc1-c1nccc(COc2ccccc2CC(Oc2nsc3cnc(C4CCC4)c(-c4ccc(OC)c(Cl)c4C)c23)C(=O)OCc2oc(=O)oc2C)n1 ; COc1ccccc1-c1nccc(COc2ccccc2CC(Oc2nsc3cnc(C4CCC4)c(-c4ccc(OC)c(Cl)c4C)c23)C(=O)O)n1 ; COc1ccccc1-c1nccc(COc2ccccc2CC(Oc2nsc3cnc(C4CCC4)c(-c4ccc(OCCN5CCOCC5)c(Cl)c4C)c23)C(=O)O)n1] 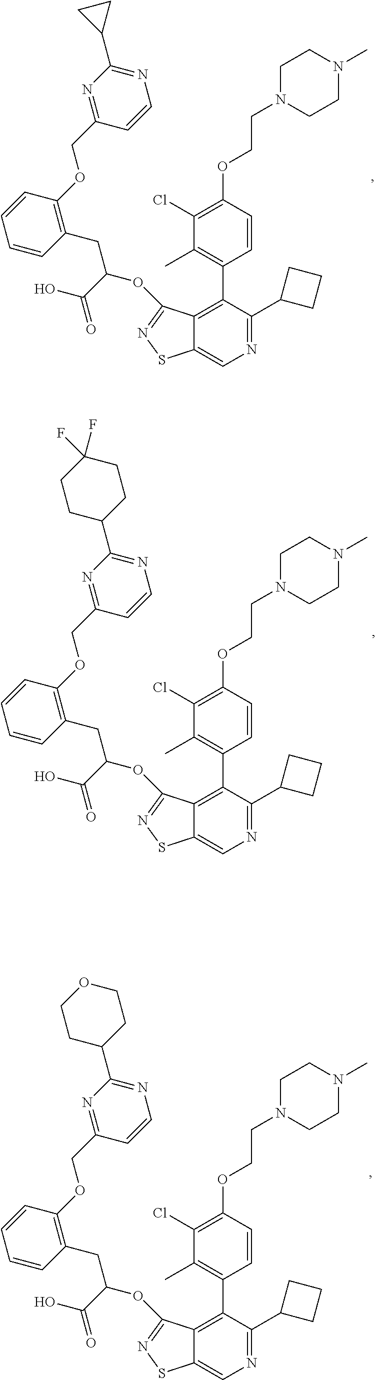 [SMILES: Cc1c(-c2c(C3CCC3)ncc3snc(OC(Cc4ccccc4OCc4ccnc(C5CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCC(F)(F)CC5)n4)C(=O)O)c23)ccc(OCCN2CCN(C)CC2)c1Cl ; Cc1c(-c2c(C3CCC3)ncc3snc(OC(Cc4ccccc4OCc4ccnc(C5CCOCC5)n4)C(=O)O)c23)ccc(OCCN2CCN(C)CC2)c1Cl] 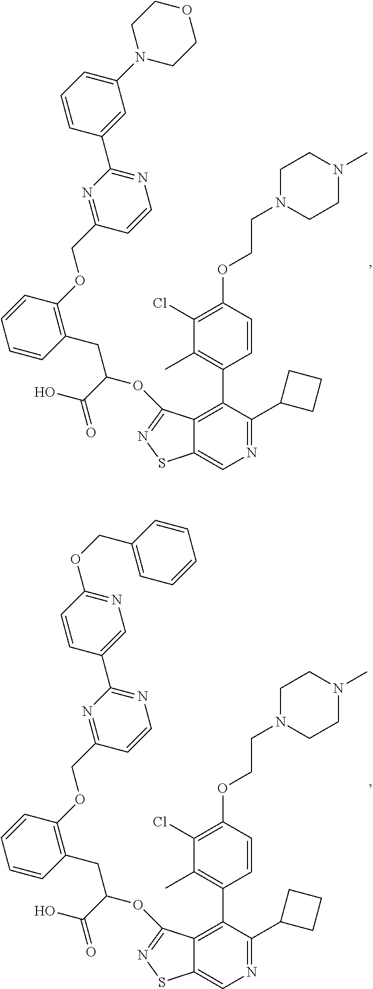 [SMILES: Cc1c(-c2c(C3CCC3)ncc3snc(OC(Cc4ccccc4OCc4ccnc(-c5cccc(N6CCOCC6)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(OCc6ccccc6)nc5)n4)C(=O)O)c23)ccc(OCCN2CCN(C)CC2)c1Cl] 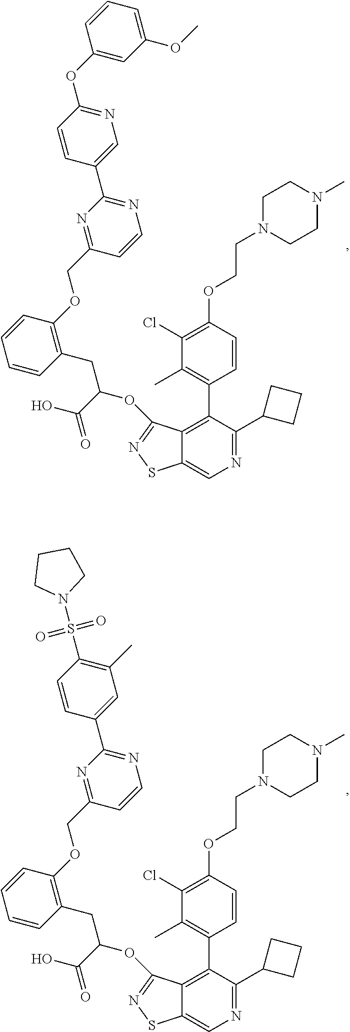 [SMILES: COc1cccc(Oc2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cn2)c1 ; Cc1cc(-c2nccc(COc3ccccc3CC(Oc3nsc4cnc(C5CCC5)c(-c5ccc(OCCN6CCN(C)CC6)c(Cl)c5C)c34)C(=O)O)n2)ccc1S(=O)(=O)N1CCCC1] 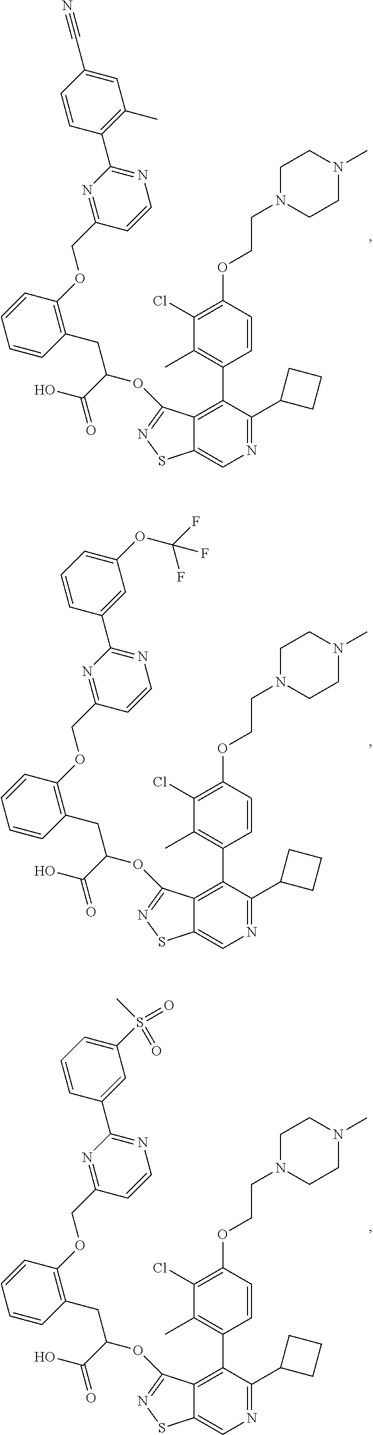 [SMILES: Cc1cc(C#N)ccc1-c1nccc(COc2ccccc2CC(Oc2nsc3cnc(C4CCC4)c(-c4ccc(OCCN5CCN(C)CC5)c(Cl)c4C)c23)C(=O)O)n1 ; Cc1c(-c2c(C3CCC3)ncc3snc(OC(Cc4ccccc4OCc4ccnc(-c5cccc(OC(F)(F)F)c5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5cccc(S(C)(=O)=O)c5)n4)C(=O)O)c23)ccc(OCCN2CCN(C)CC2)c1Cl] 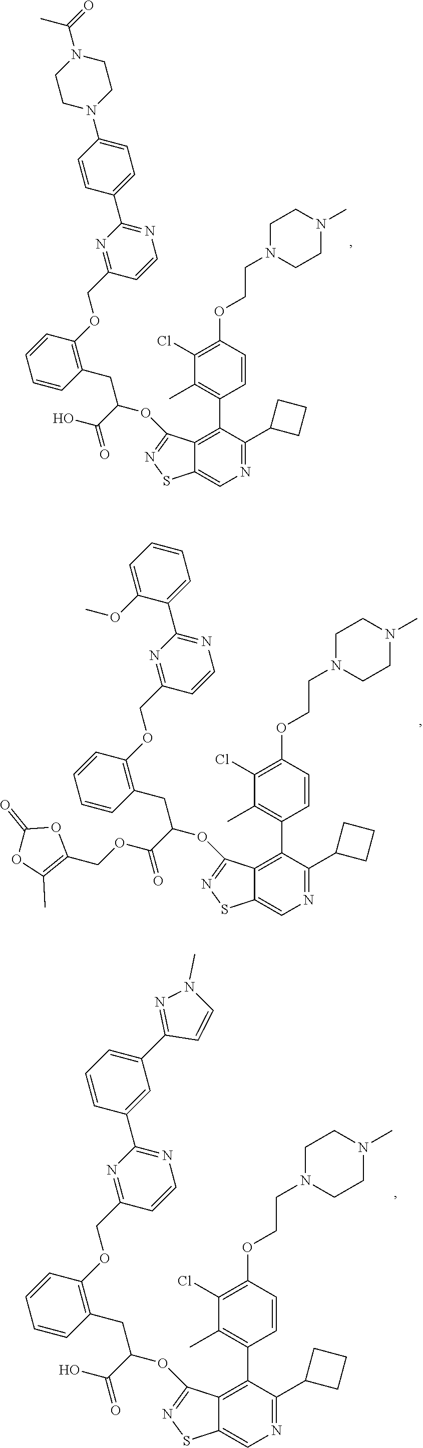 [SMILES: CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)cc2)CC1 ; COc1ccccc1-c1nccc(COc2ccccc2CC(Oc2nsc3cnc(C4CCC4)c(-c4ccc(OCCN5CCN(C)CC5)c(Cl)c4C)c23)C(=O)OCc2oc(=O)oc2C)n1 ; Cc1c(-c2c(C3CCC3)ncc3snc(OC(Cc4ccccc4OCc4ccnc(-c5cccc(-c6ccn(C)n6)c5)n4)C(=O)O)c23)ccc(OCCN2CCN(C)CC2)c1Cl] 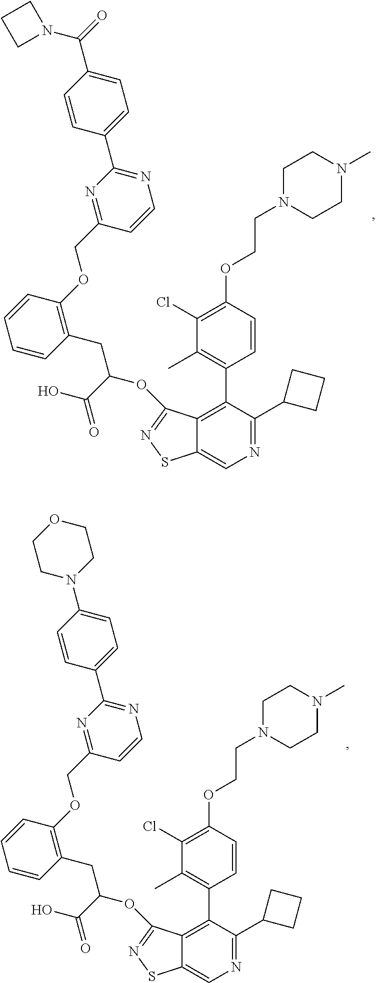 [SMILES: Cc1c(-c2c(C3CCC3)ncc3snc(OC(Cc4ccccc4OCc4ccnc(-c5ccc(C(=O)N6CCC6)cc5)n4)C(=O)O)c23)ccc(OCCN2CCN(C)CC2)c1Cl ; Cc1c(-c2c(C3CCC3)ncc3snc(OC(Cc4ccccc4OCc4ccnc(-c5ccc(N6CCOCC6)cc5)n4)C(=O)O)c23)ccc(OCCN2CCN(C)CC2)c1Cl] 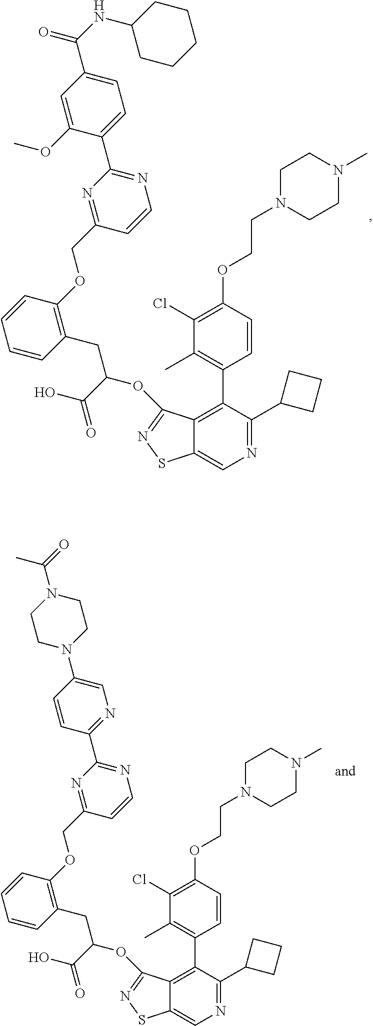 [SMILES: COc1cc(C(=O)NC2CCCCC2)ccc1-c1nccc(COc2ccccc2CC(Oc2nsc3cnc(C4CCC4)c(-c4ccc(OCCN5CCN(C)CC5)c(Cl)c4C)c23)C(=O)O)n1 ; CC(=O)N1CCN(c2ccc(-c3nccc(COc4ccccc4CC(Oc4nsc5cnc(C6CCC6)c(-c6ccc(OCCN7CCN(C)CC7)c(Cl)c6C)c45)C(=O)O)n3)nc2)CC1] 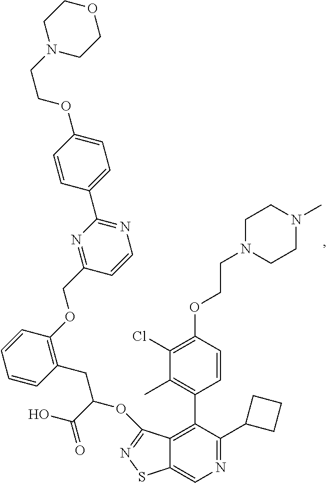 [SMILES: Cc1c(-c2c(C3CCC3)ncc3snc(OC(Cc4ccccc4OCc4ccnc(-c5ccc(OCCN6CCOCC6)cc5)n4)C(=O)O)c23)ccc(OCCN2CCN(C)CC2)c1Cl]
or a pharmaceutically acceptable salt thereof.

52. A compound selected from: 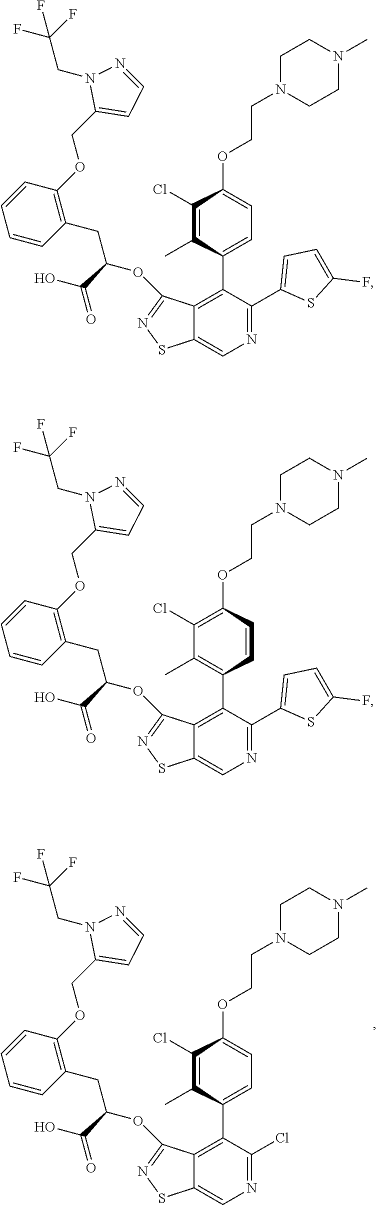 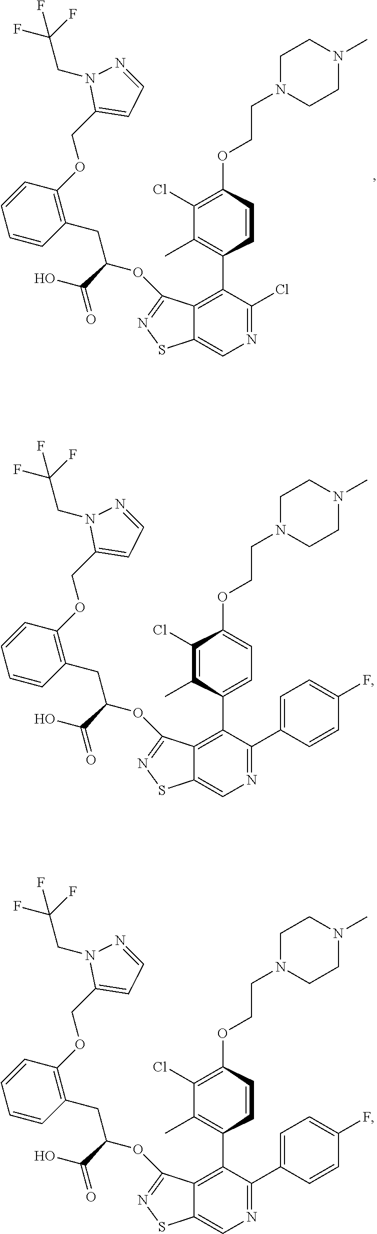 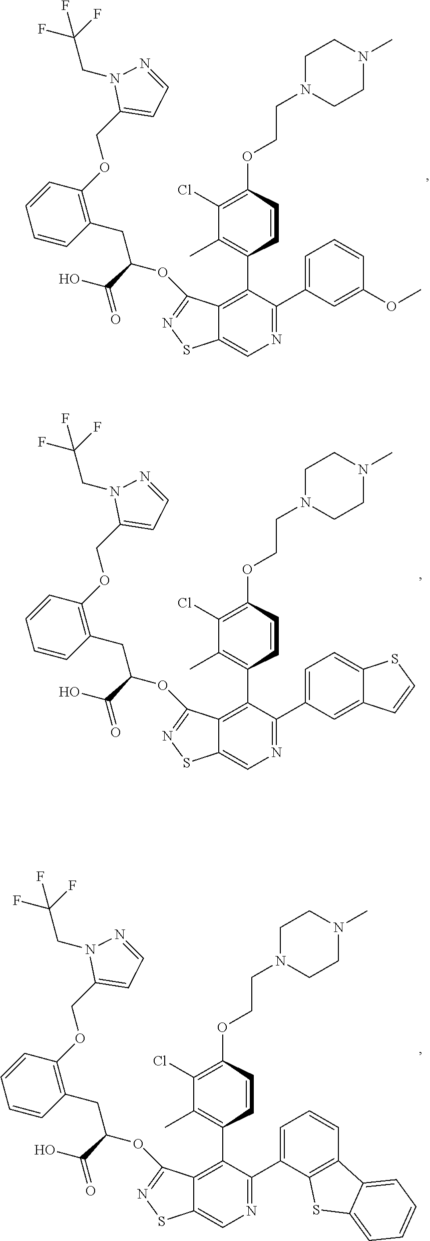 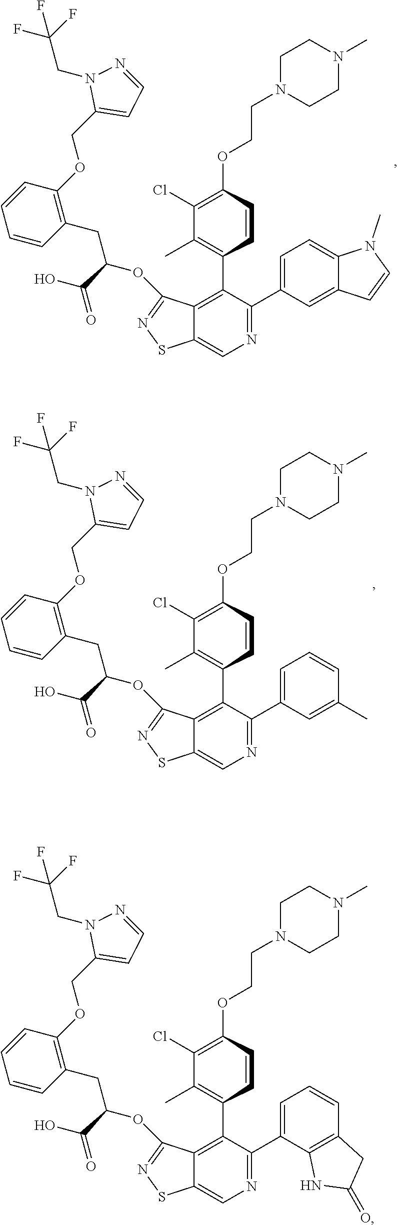 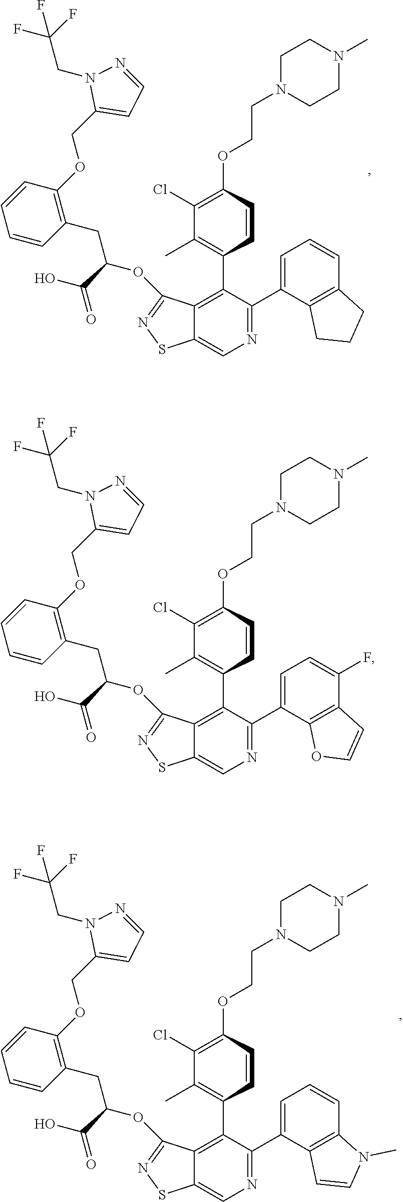 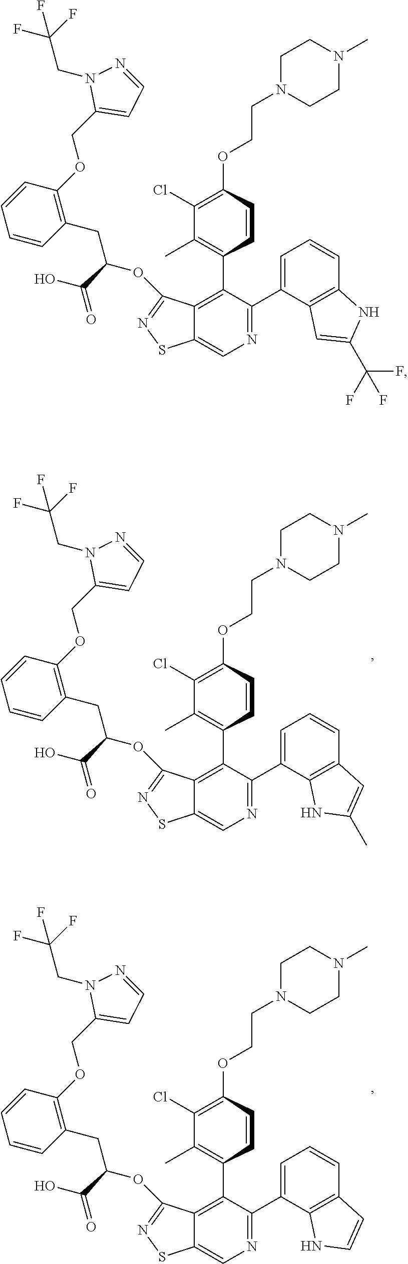 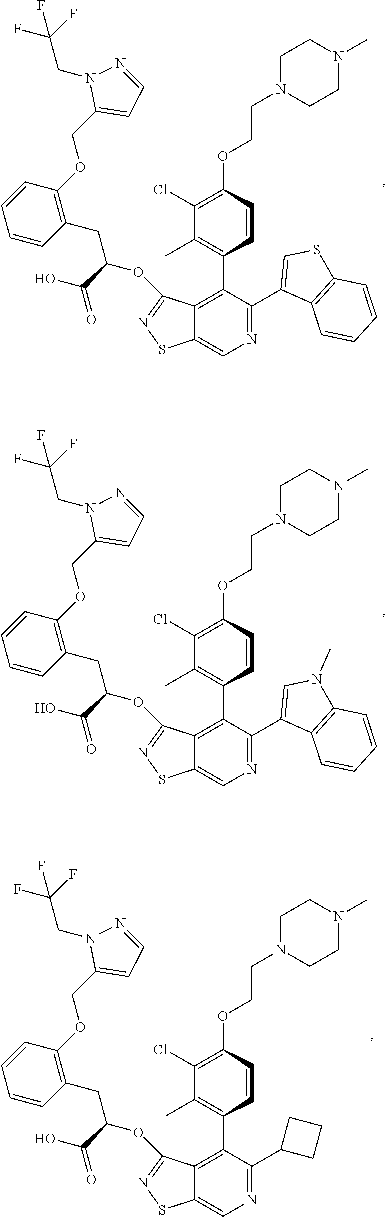 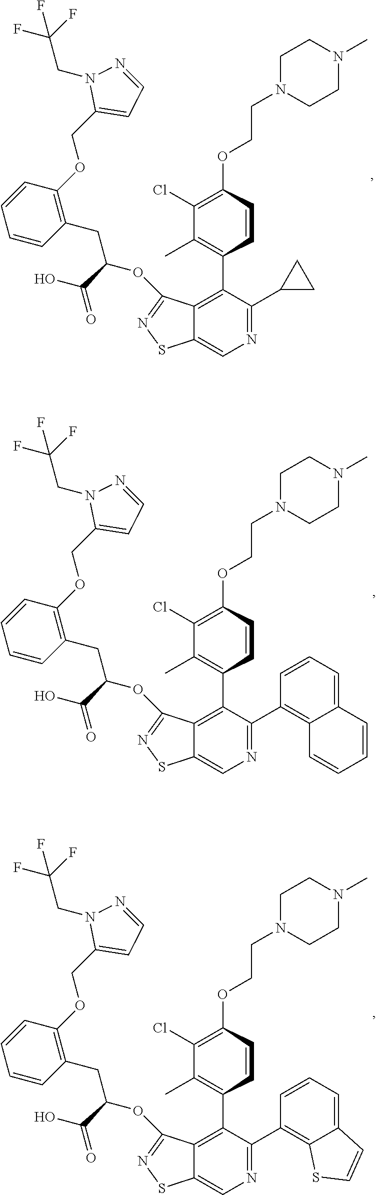 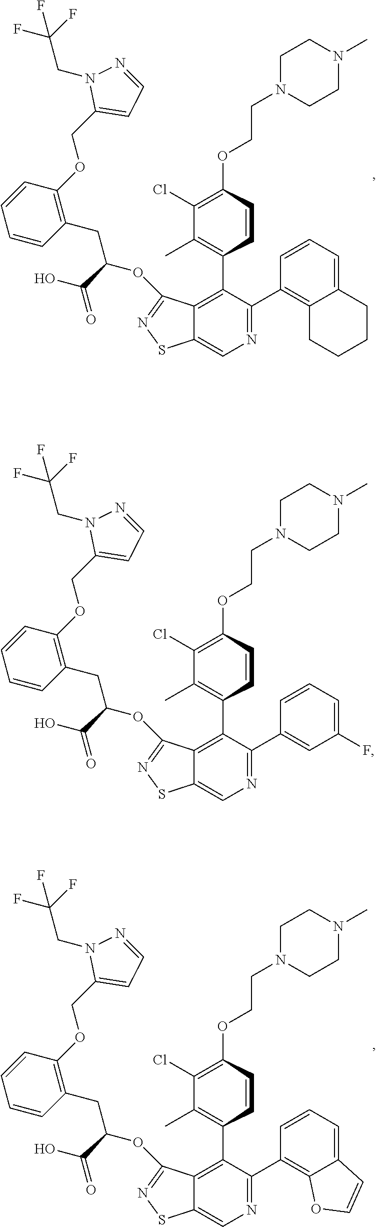 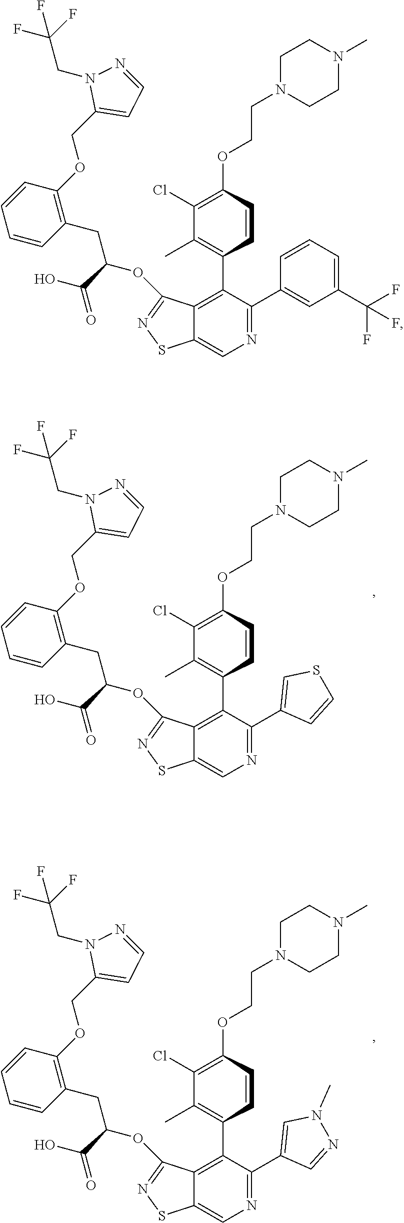 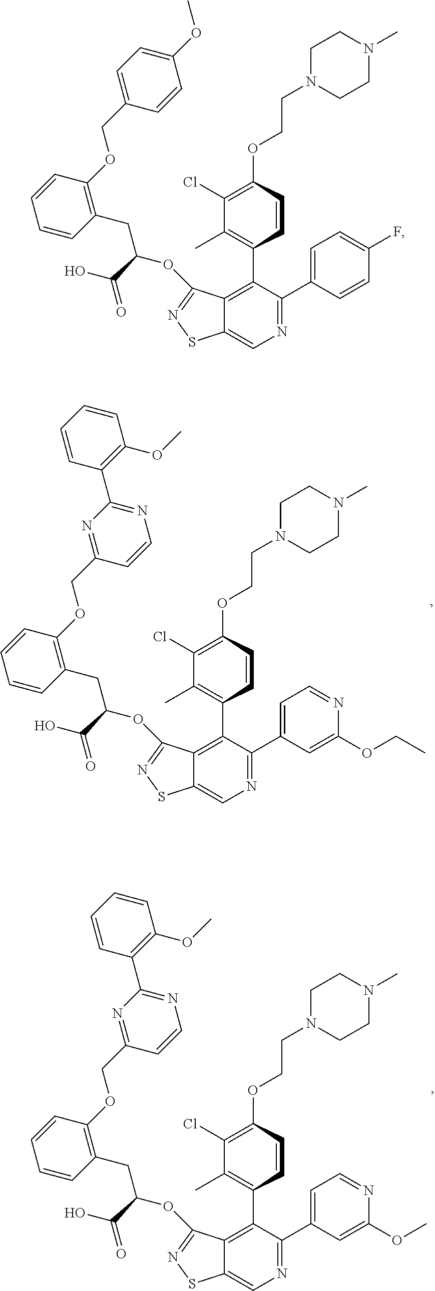 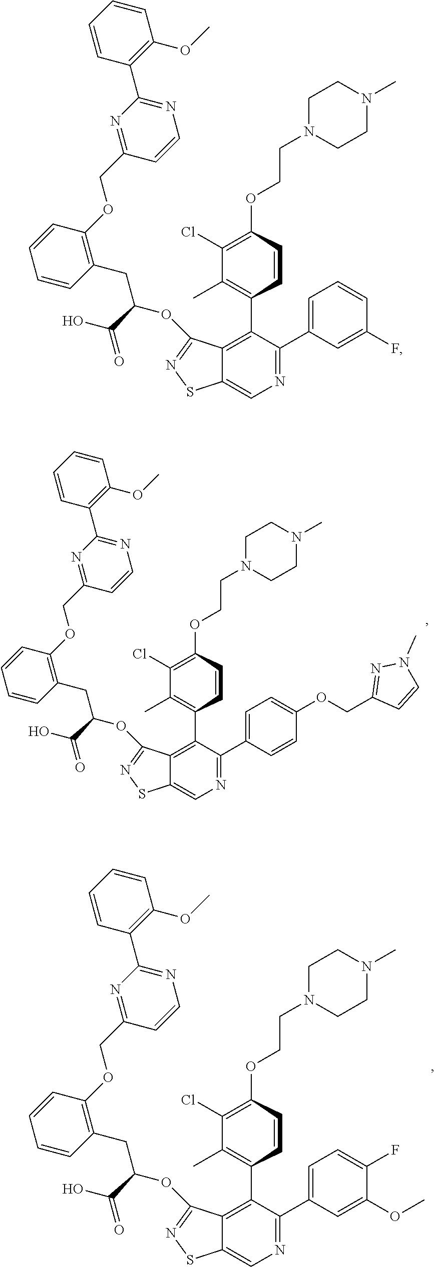 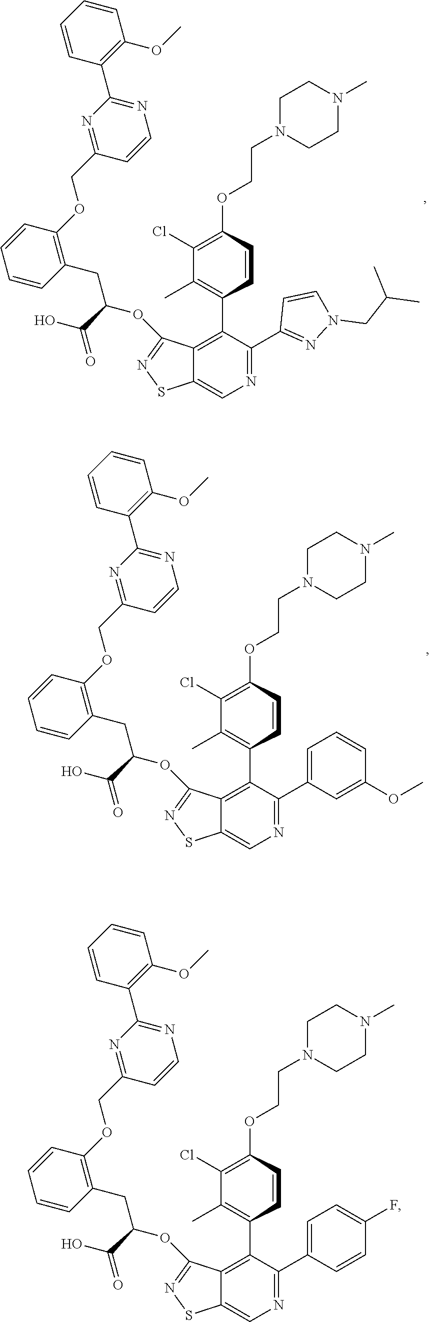 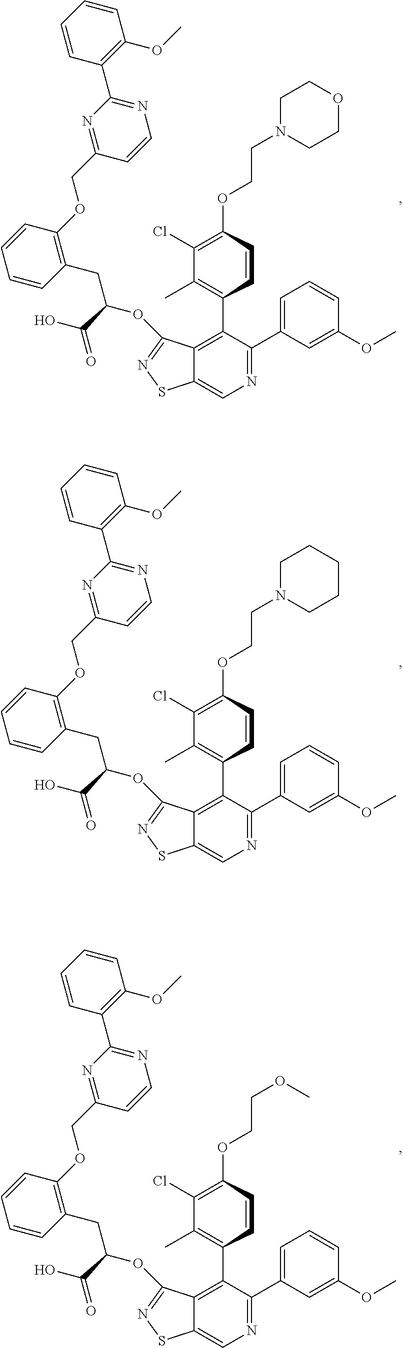 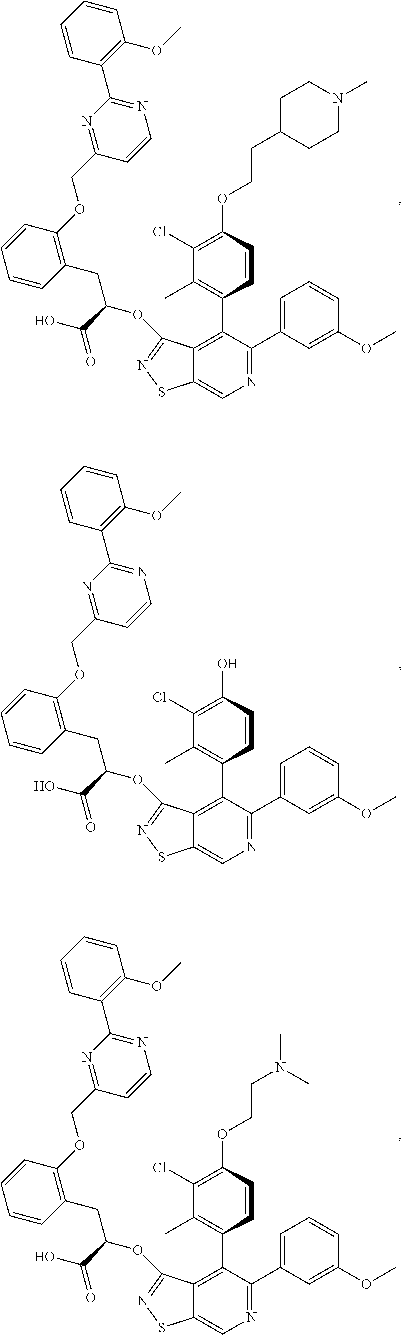 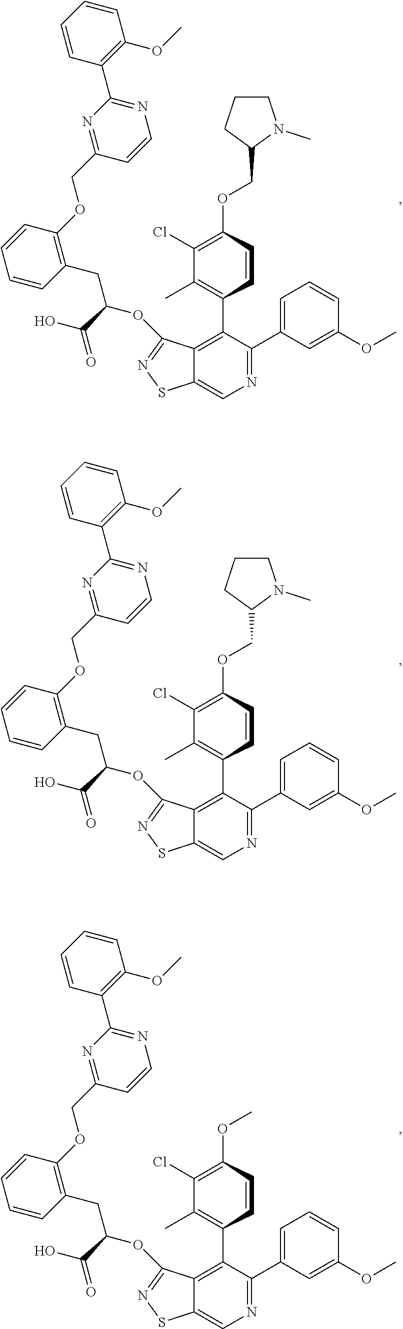 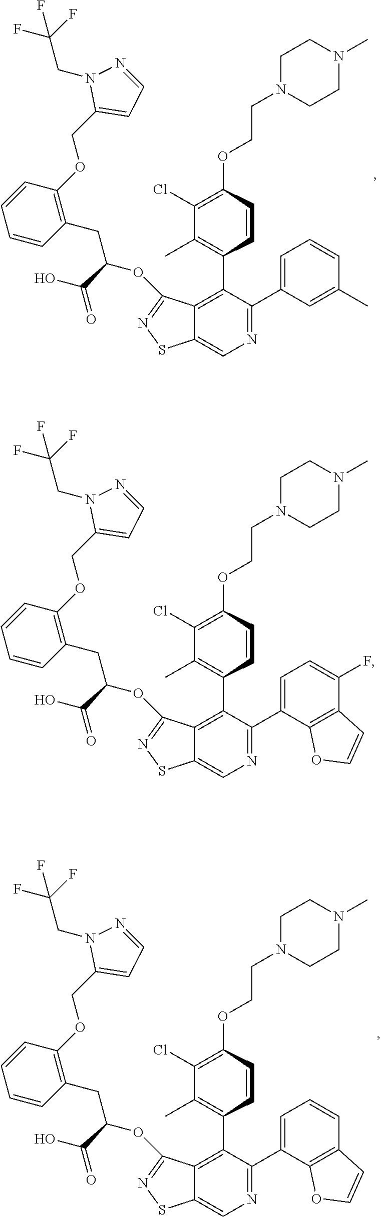 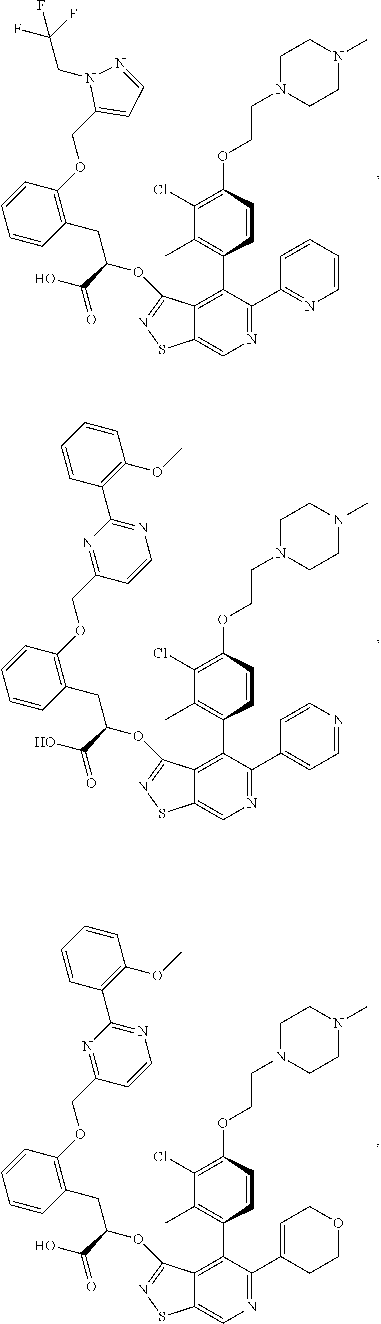 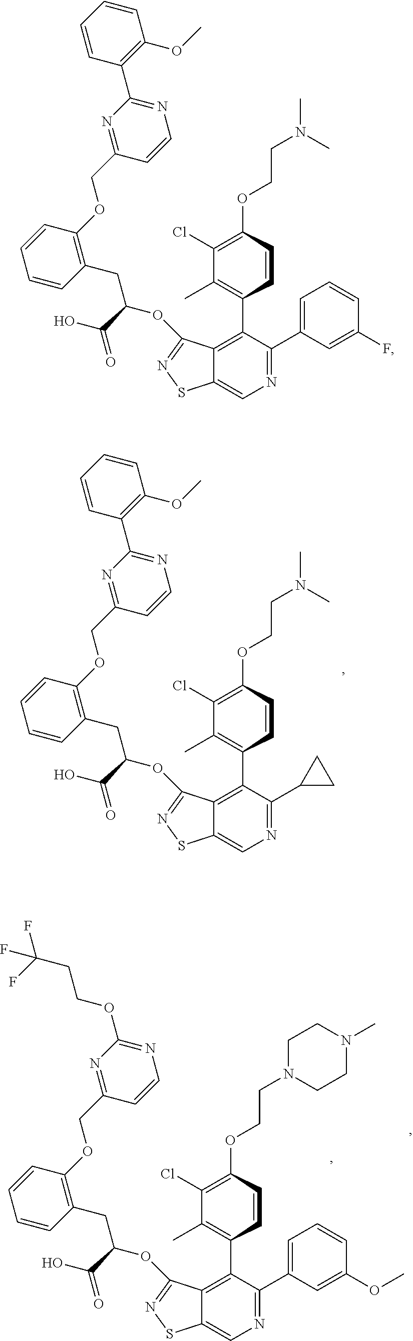 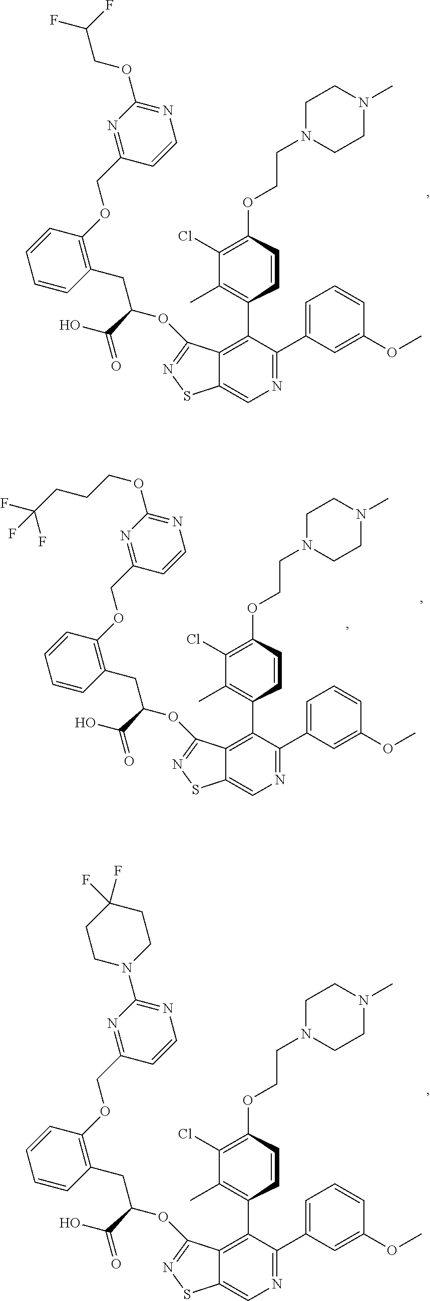 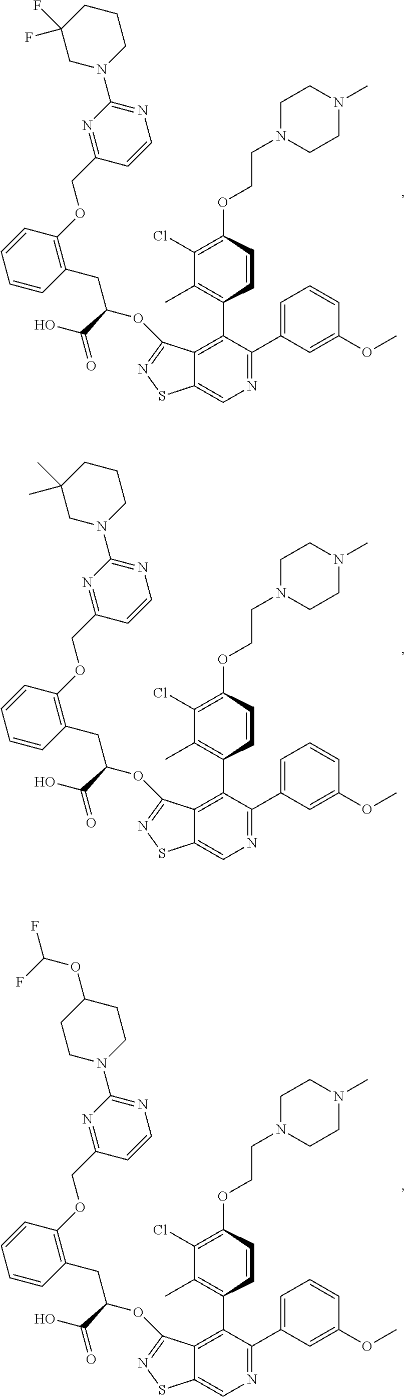 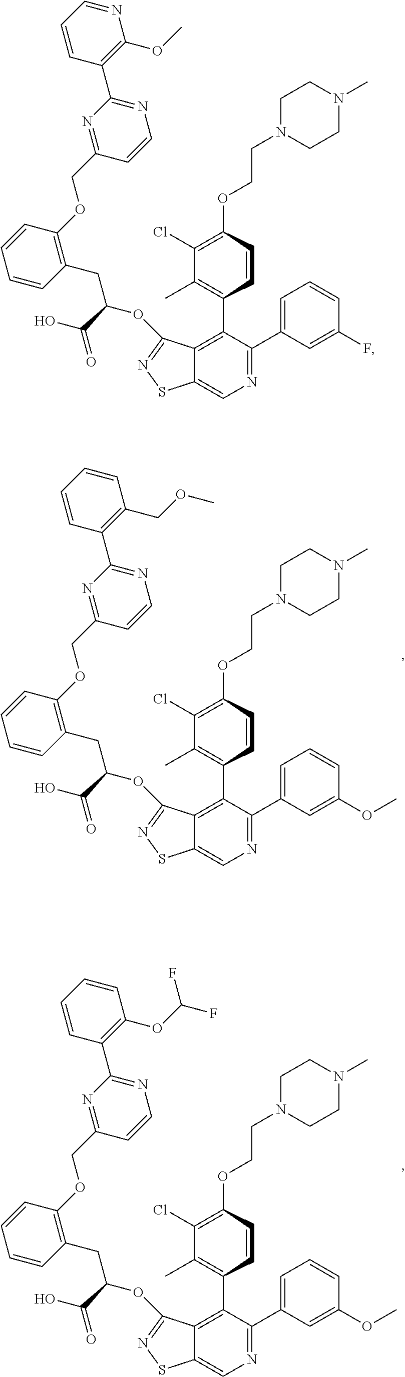 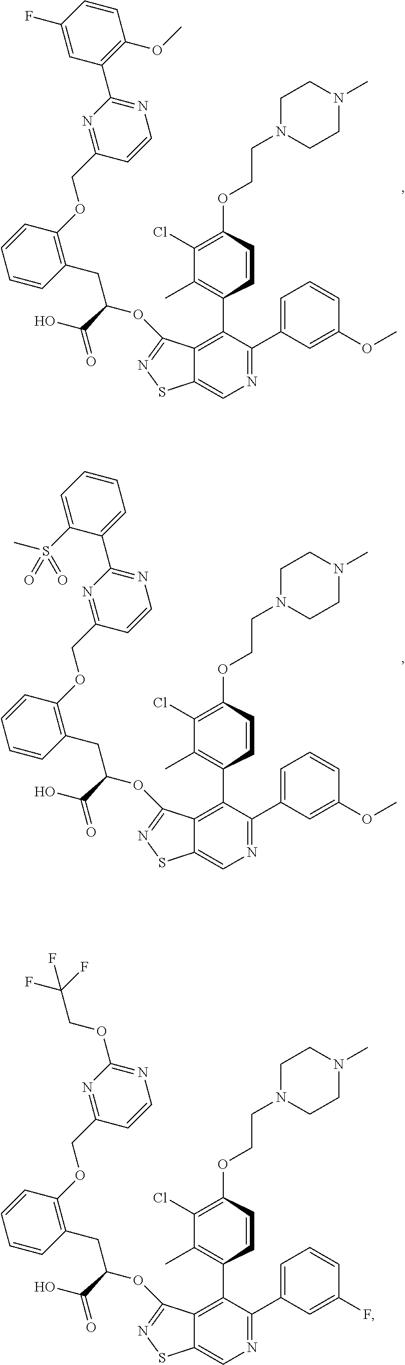 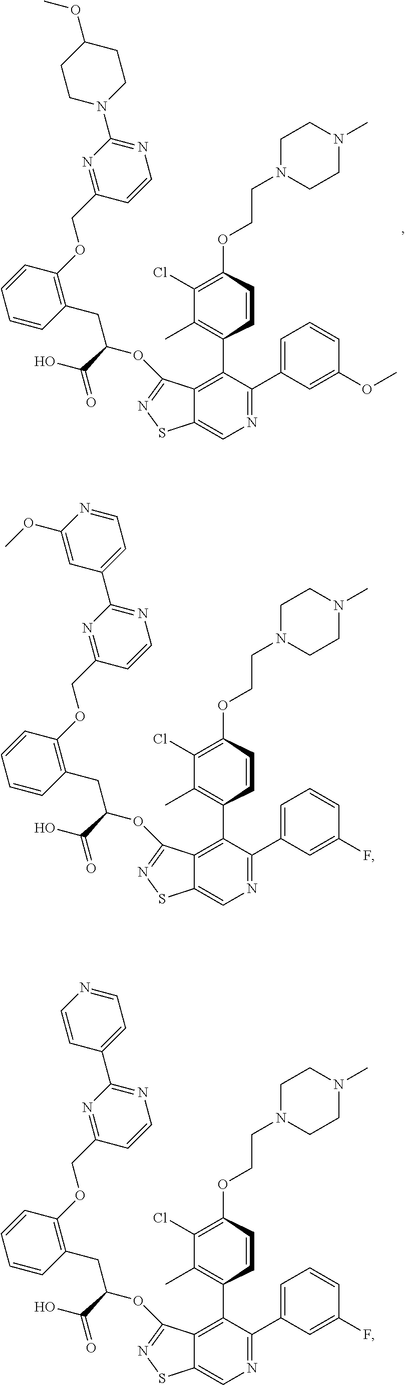 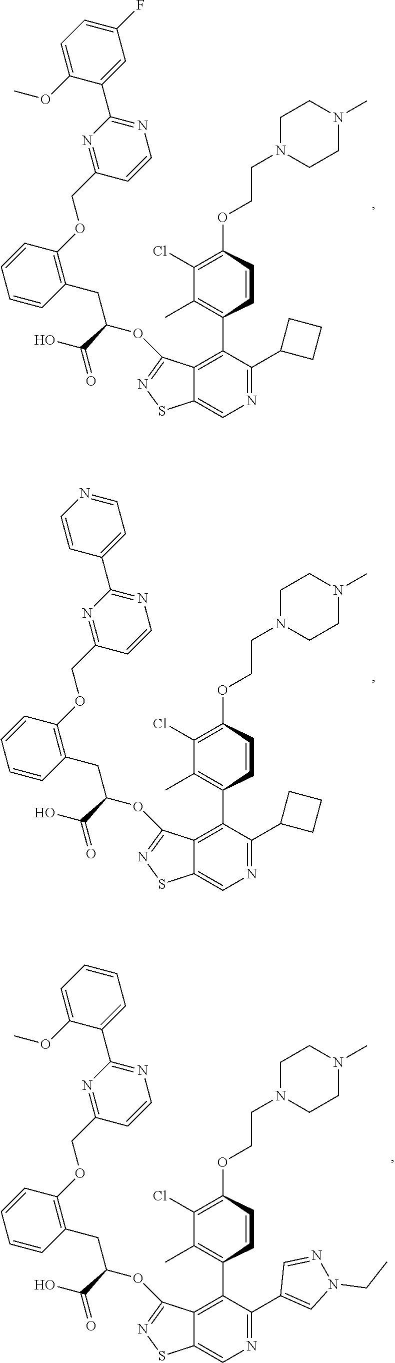 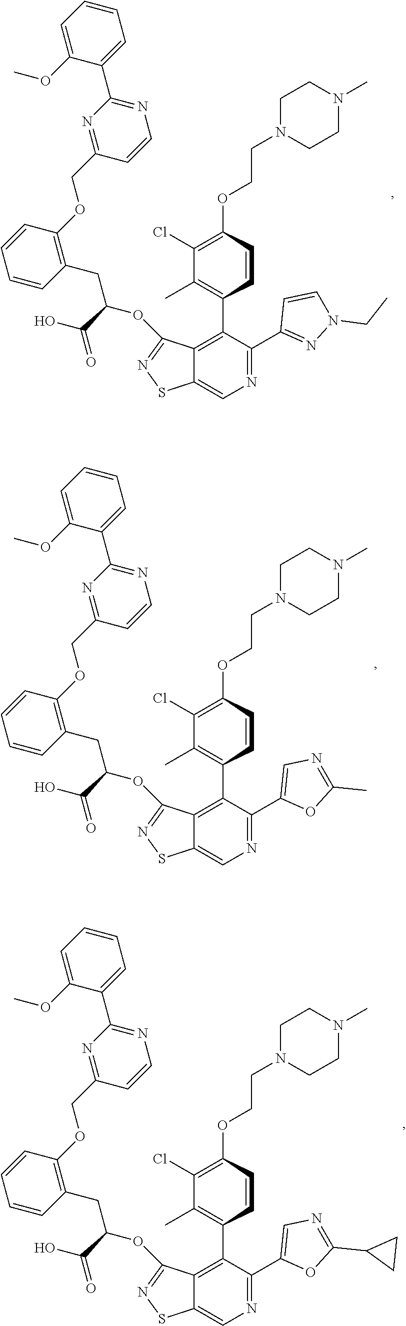 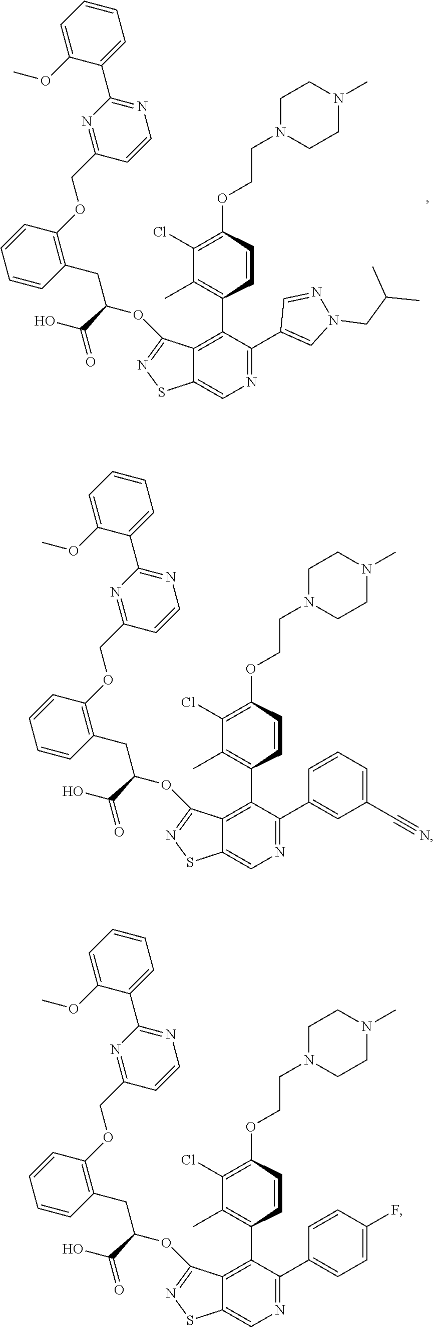 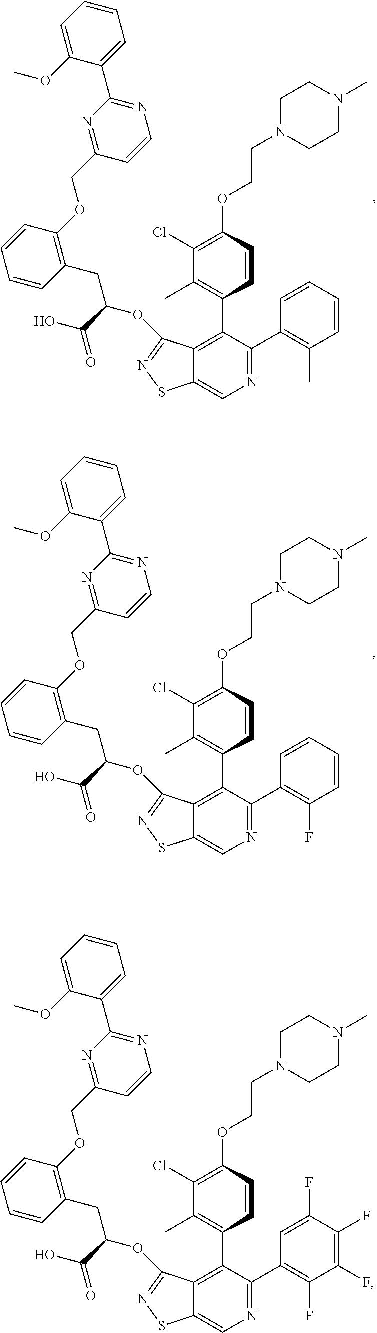 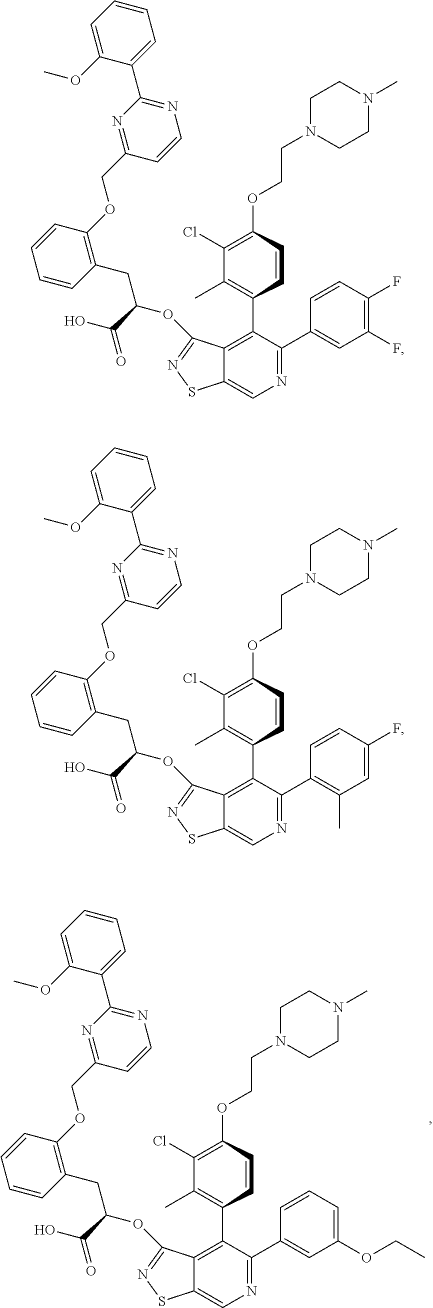 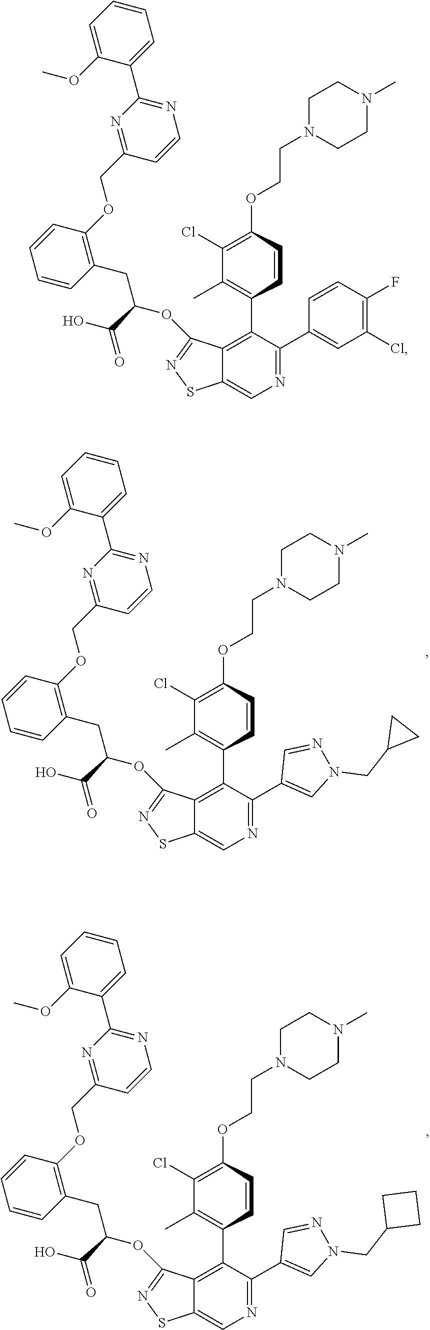 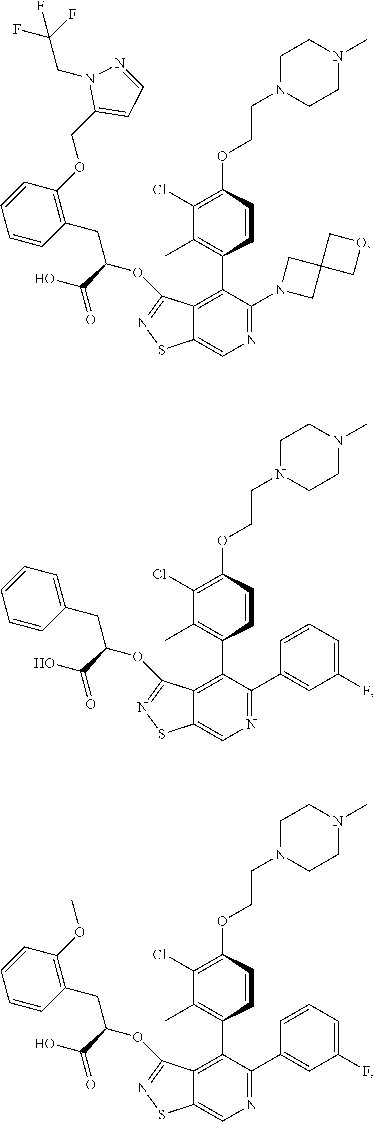 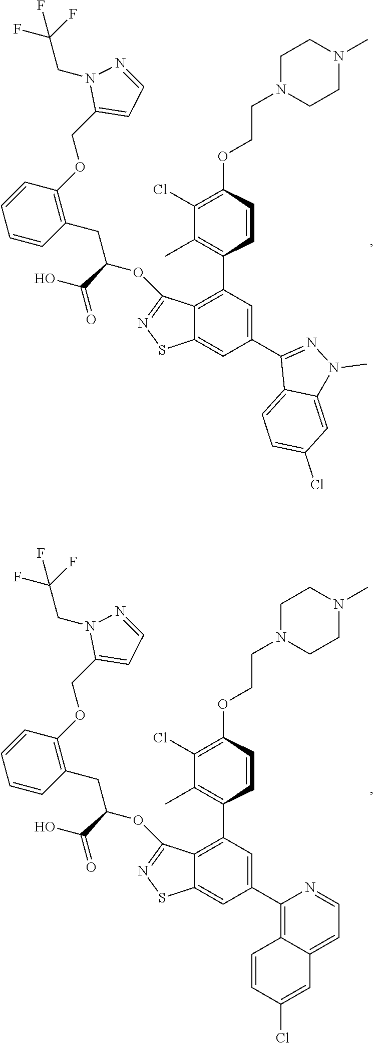 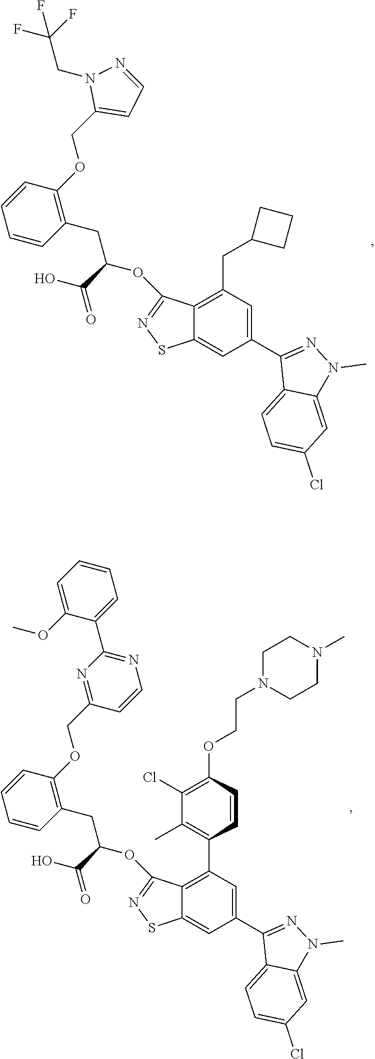 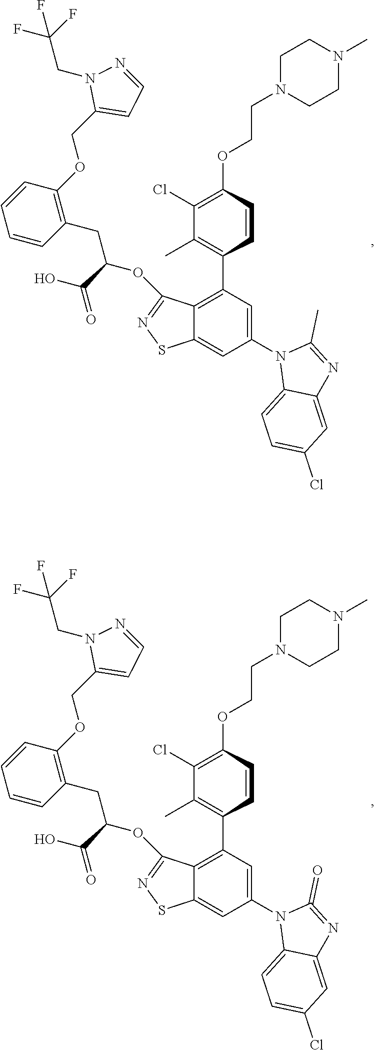 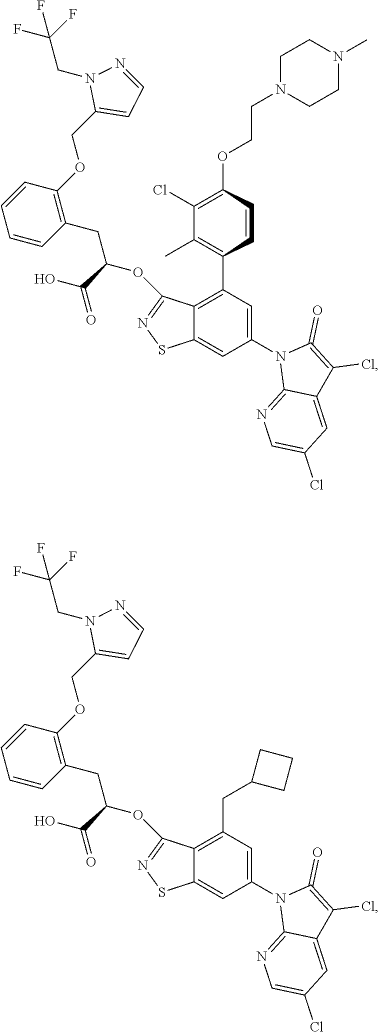 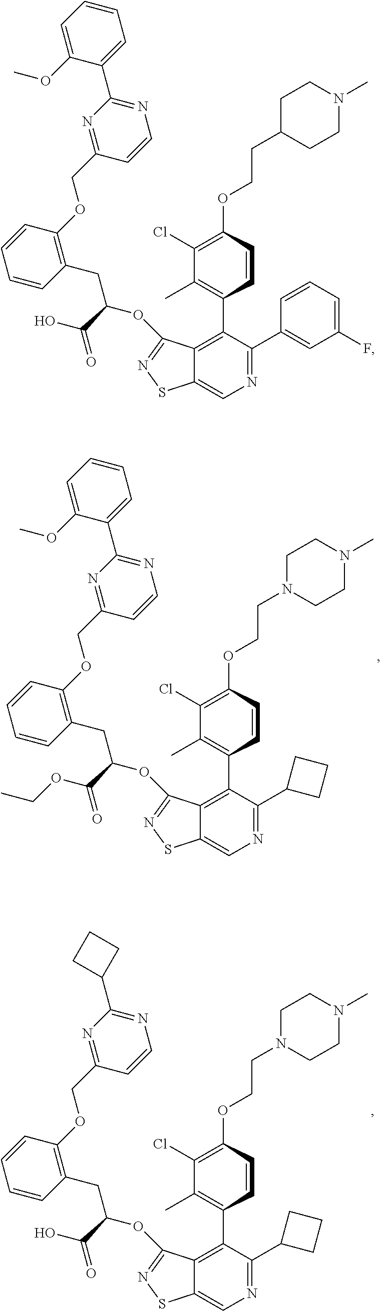 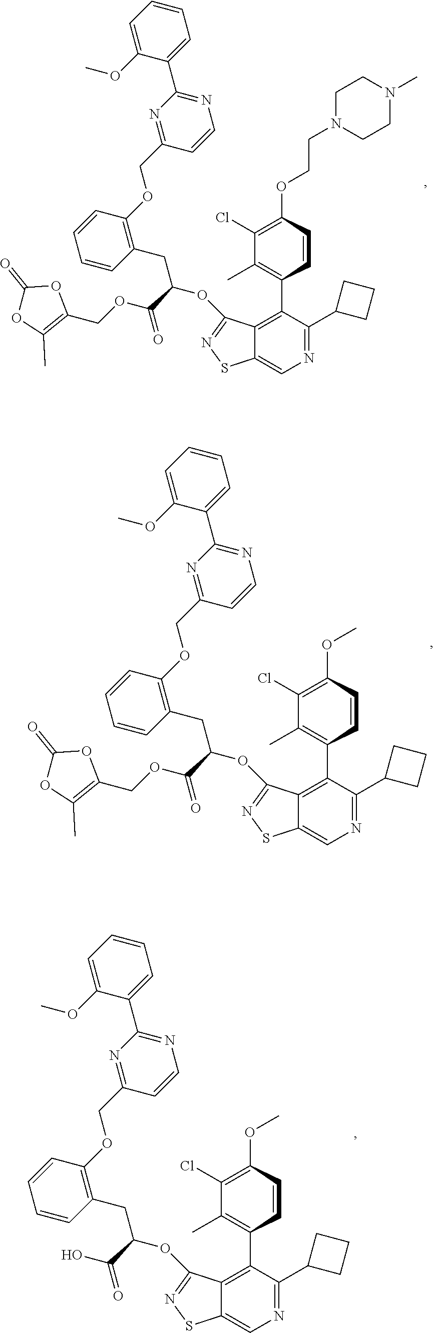 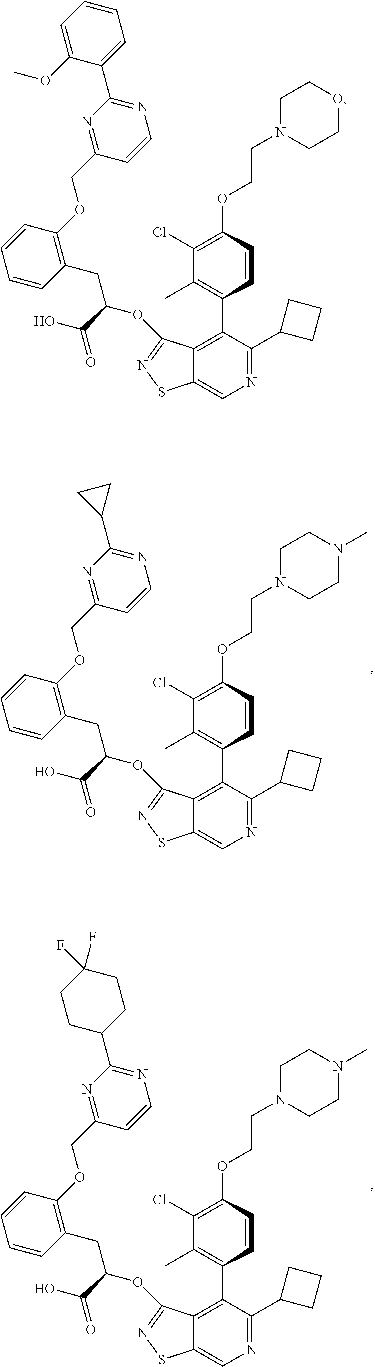 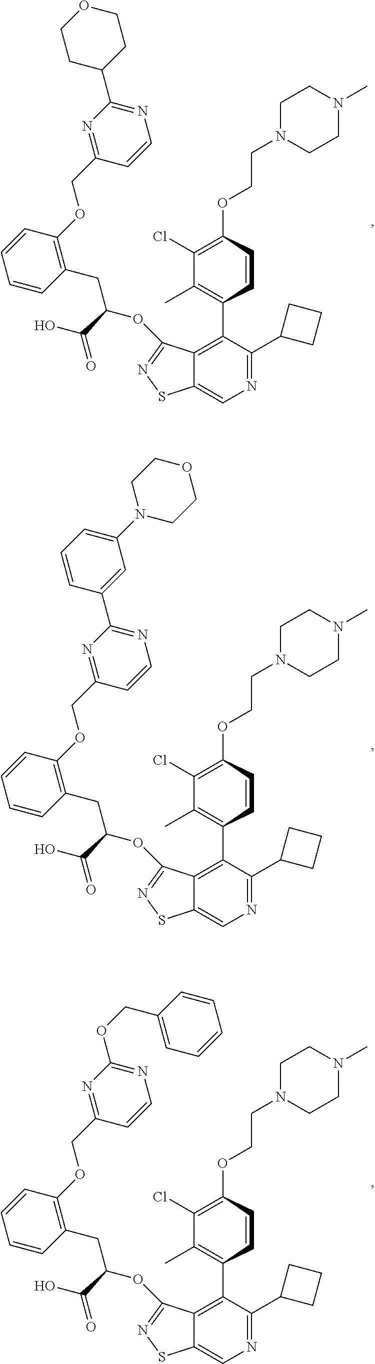 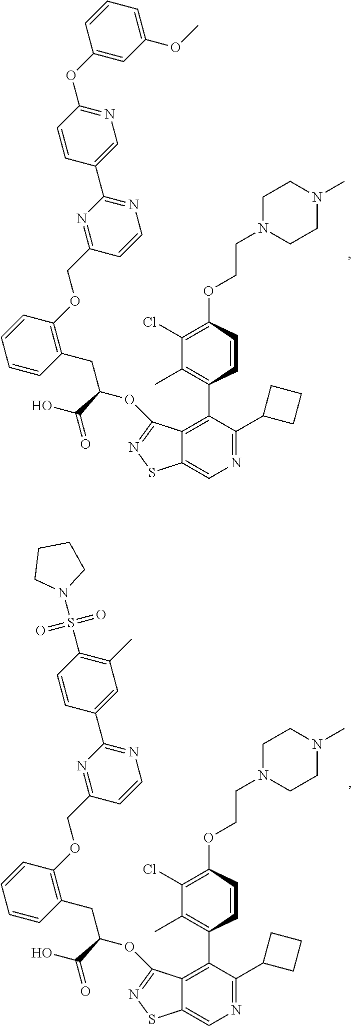 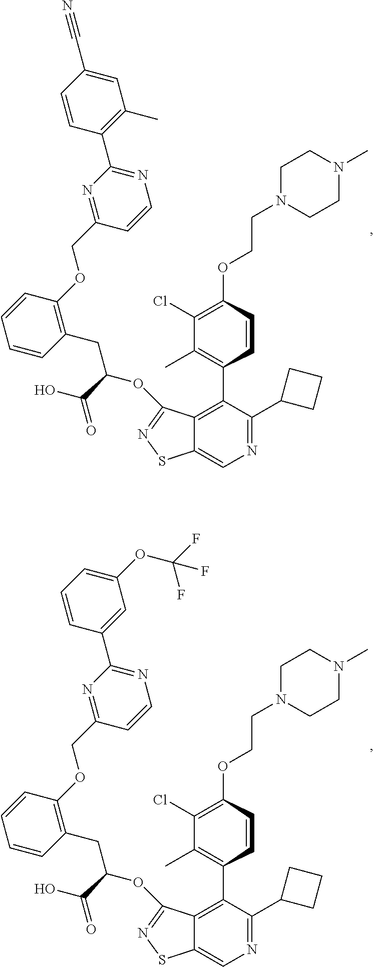 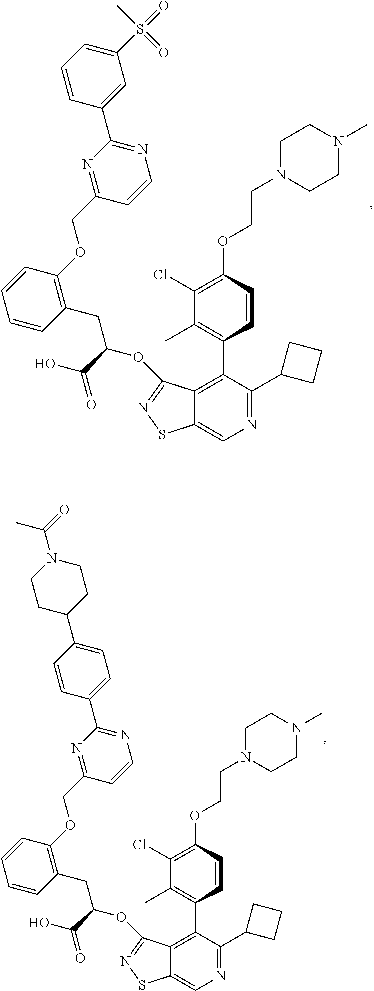 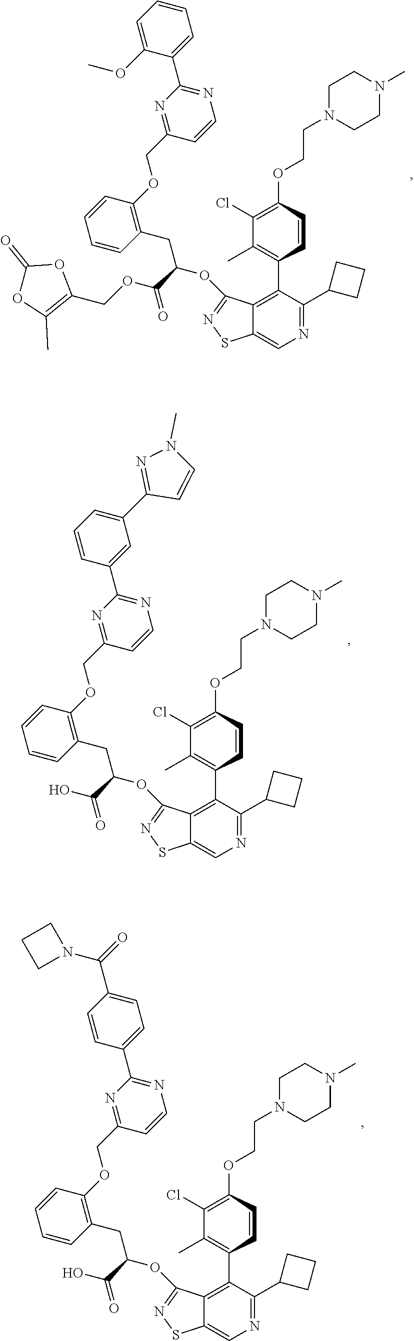 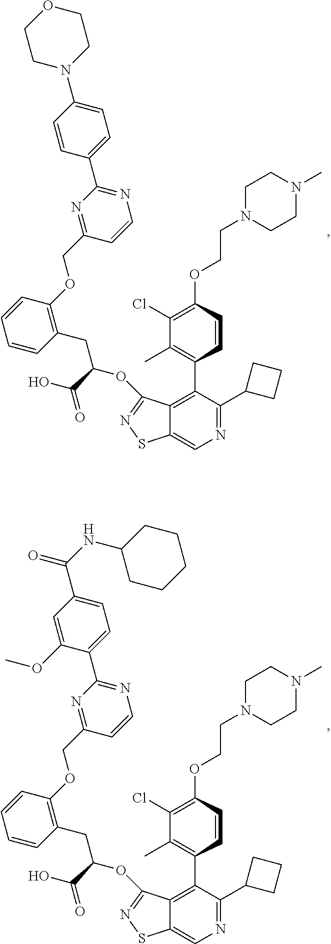 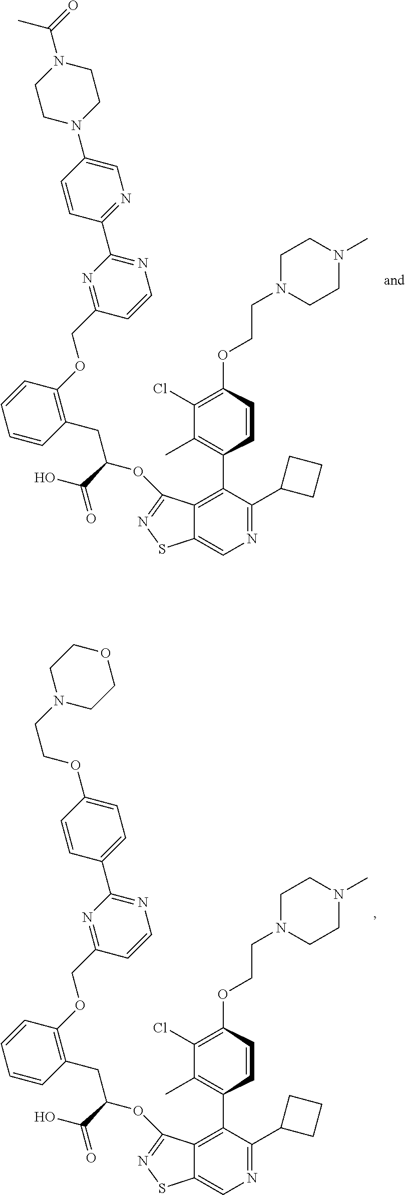
or a pharmaceutically acceptable salt thereof.

53. A compound selected from: 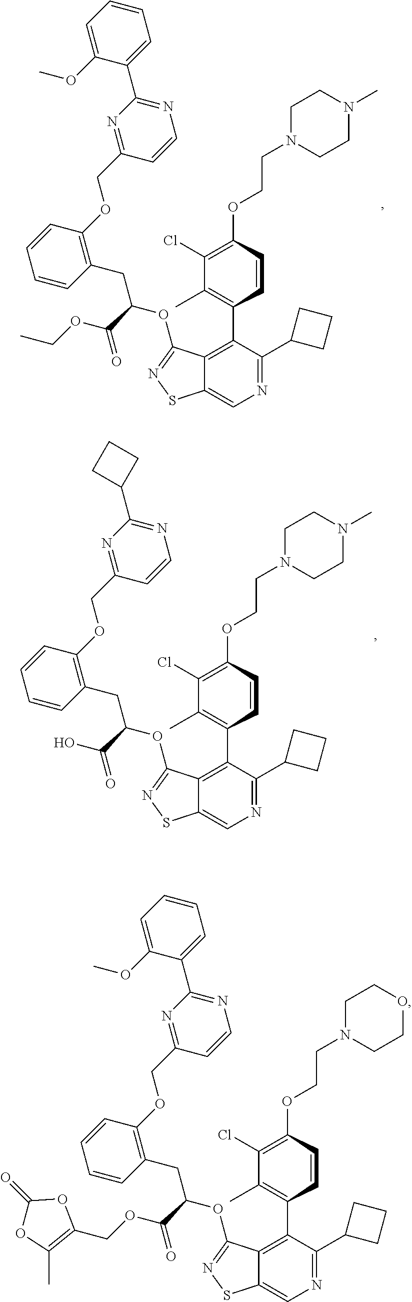 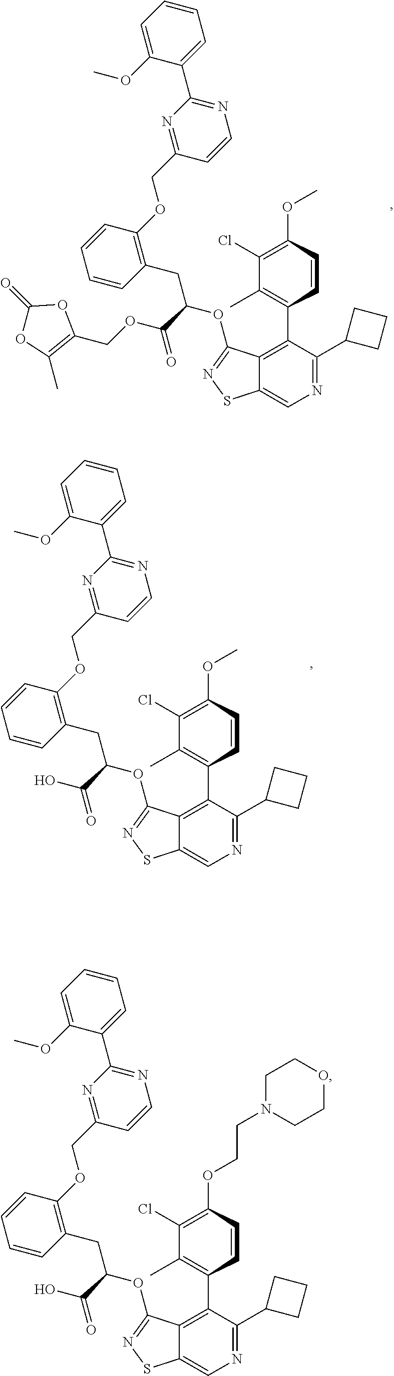 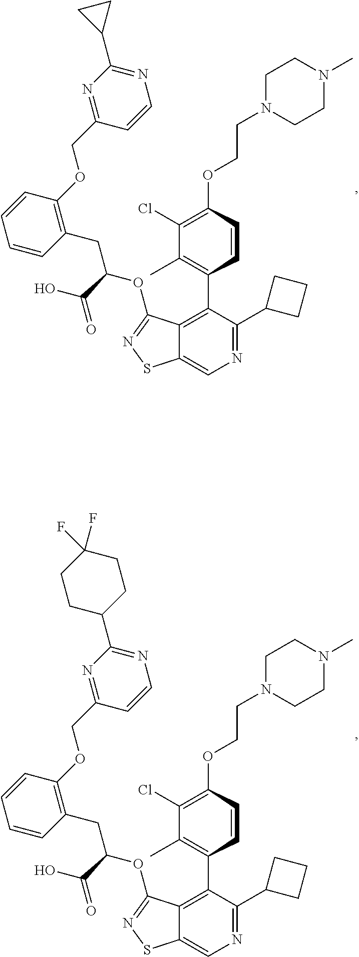 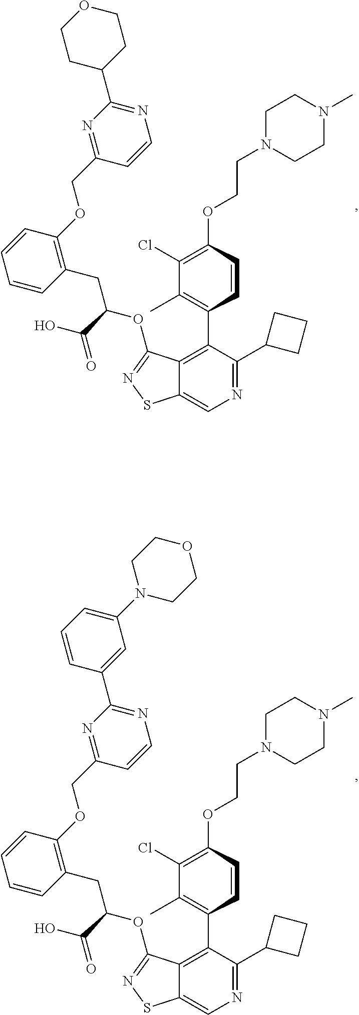 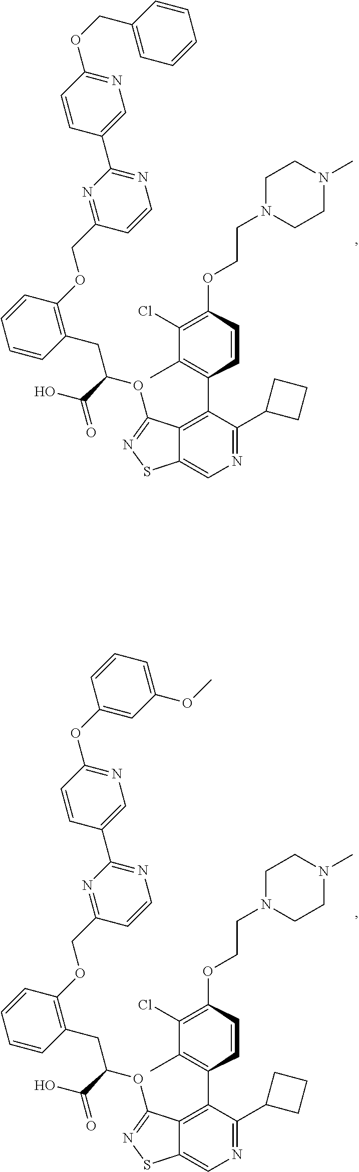 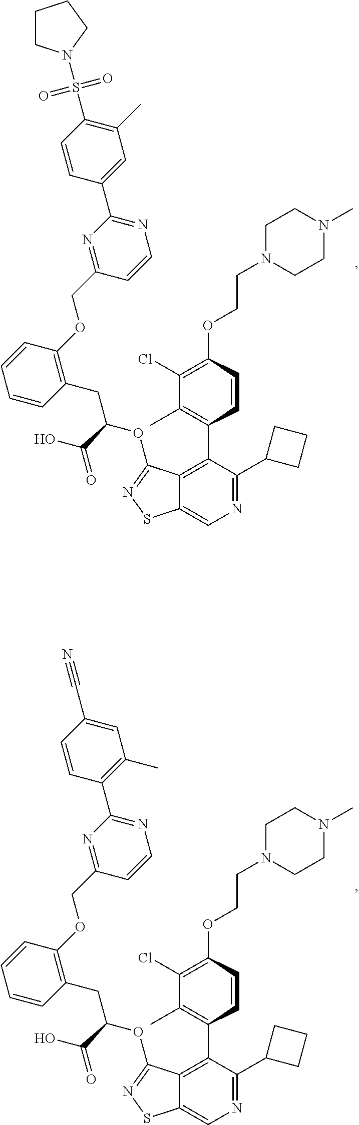 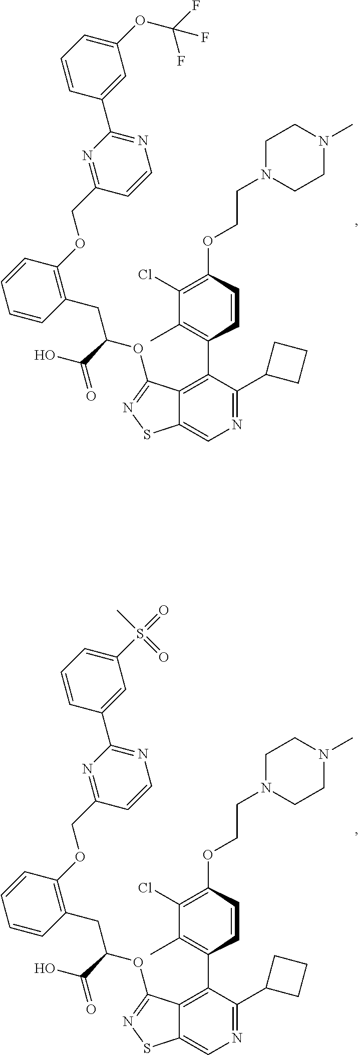 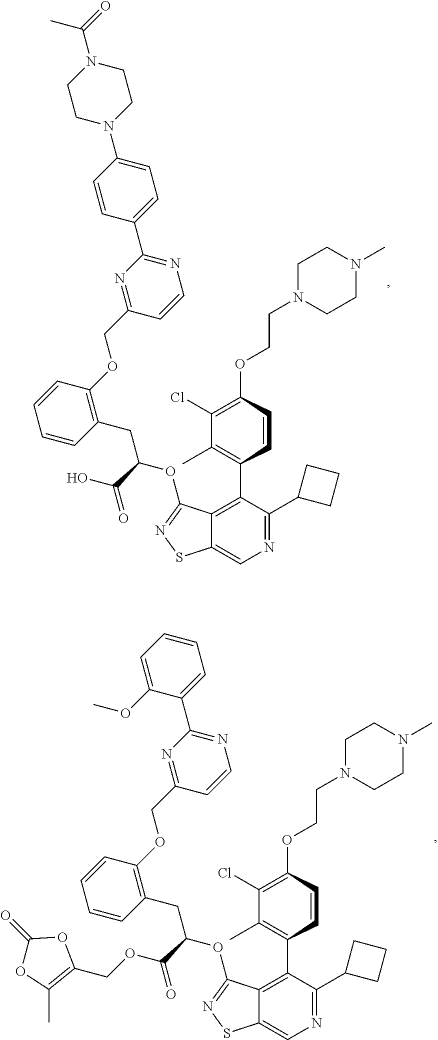 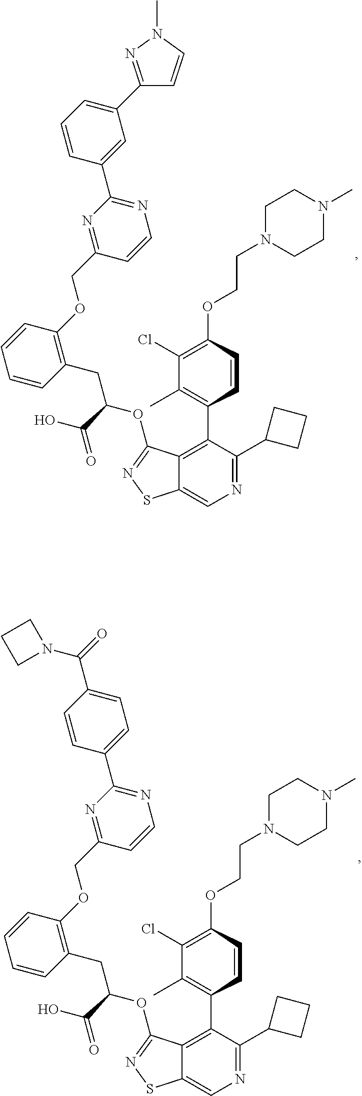 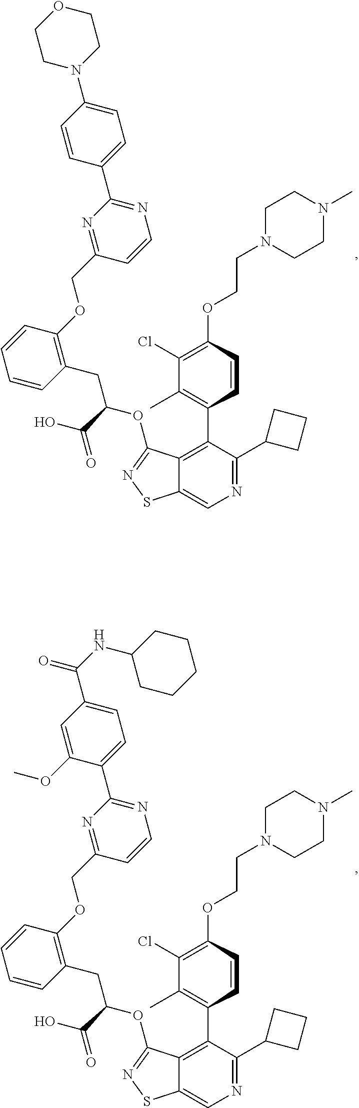 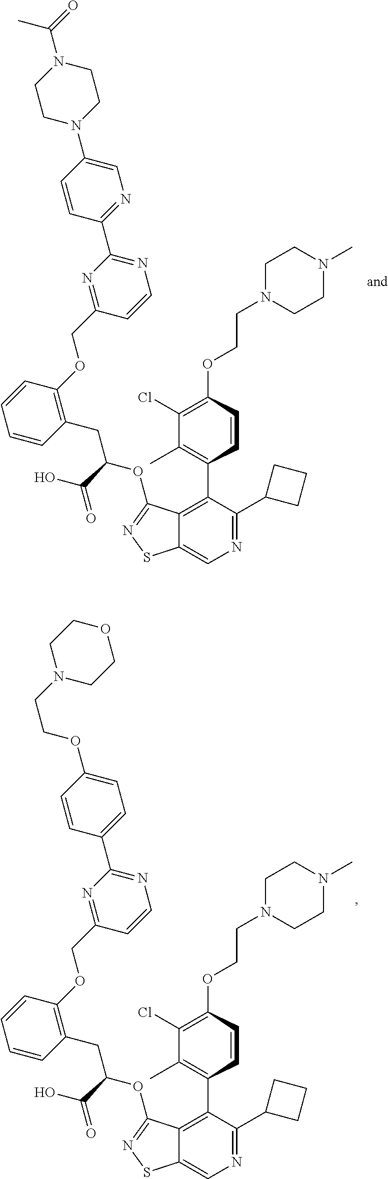
or a pharmaceutically acceptable salt thereof.

54. A compound having the structure of Formula VIII: 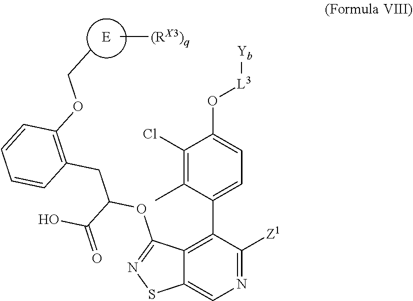
or a pharmaceutically acceptable salt thereof,
wherein:
E is heteroaryl;
L³ is —CH₂—, or —CH₂CH₂—;
Y_{b} is H, heterocyclyl, —N(CH₃)₂, —N(CH₂CH₃)₂, —CH₂N(CH₃)₂ or —CH₂N(CH₂CH₃)₂; or
L³ is absent and Y_{b} is H;
Z¹ is aryl, heteroaryl, cycloalkyl or heterocyclyl, wherein each of said aryl, heteroaryl, cycloalkyl and heterocyclyl is optionally substituted with one or more R^{Z1};
R^{X3} in each instance is independently aryl, heteroaryl, heterocyclyl, C₁-C₆ alkyl, C₁-C₆ alkoxy, C₁-C₆ haloalkyl, C₁-C₆ hydroxyalkyl, hydroxy, halogen, amino, nitro or cyano, wherein each of said aryl, heteroaryl, heterocyclyl and C₁-C₆ alkoxy is optionally substituted with one, two, three or four R^{X3}a;
R^{X3a} in each instance is independently C₁-C₆ alkyl, C₁-C₆ alkoxy, C₁-C₆ haloalkyl, C₁-C₆ hydroxyalkyl, hydroxy, halogen, amino, nitro or cyano;
R^{Z1} in each instance is independently aryl, heteroaryl, cycloalkyl, heterocyclyl, oxo, halogen, hydroxy, N(R^{Z2})(R^{Z2}), C₁-C₆ alkyl, C₁-C₆ aminoalkyl, C₁-C₆ alkoxy, C₁-C₆ hydroxyalkyl, C₁-C₆ haloalkyl, C₂-C₆ alkenyl, C₂-C₆ alkynyl, nitro or cyano, wherein each of said aryl, heteroaryl, cycloalkyl, heterocyclyl, C₁-C₆ alkyl, C₁-C₆ aminoalkyl, C₁-C₆ alkoxy, C₂-C₆ alkenyl and C₂-C₆ alkynyl is optionally substituted with one or more R^{Z3};
R^{Z2} in each instance is independently H, aryl, heteroaryl, cycloalkyl, heterocyclyl, C₁-C₆ alkyl, C₁-C₆ alkoxy, C₁-C₆ haloalkyl or C₁-C₆ hydroxyalkyl, wherein each of said aryl, heteroaryl, cycloalkyl, heterocyclyl, C₁-C₆ alkyl and C₁-C₆ alkoxy is optionally substituted with one or more R^{Z3}; or
two R^{Z2}, together with the N to which they are connected, form a heterocycle or heteroaryl, wherein each of said heterocycle and heteroaryl is optionally substituted with one, two, three or four R^{Z3};
R^{Z3} in each instance is independently aryl, heteroaryl, cycloalkyl, heterocyclyl, C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano, wherein each of said aryl, heteroaryl, cycloalkyl and heterocyclyl is optionally substituted with one or more R^{Z4};
R^{Z4} in each instance is independently C₁-C₆ alkyl, C₁-C₆ aminoalkyl, C₁-C₆ alkylamino, C₁-C₆ alkoxy, C₁-C₆ haloalkyl, C₁-C₆ hydroxyalkyl, hydroxy, halogen, amino, nitro or cyano; and
q is 0, 1, 2, 3 or 4.

55. The compound of claim 46, wherein the compound has an MCL1 IC₅₀ of about 100 nM or lower.

56. The compound of claim 46 or 47, wherein the compound has an average IC₅₀ for the drug sensitive cell lines of Table 3 of 1 μM or lower.

57. The compound of claim 46, 47 or 48, wherein the compound has an average IC₅₀ for the drug-sensitive cell lines of Table 3 that is at least about 10-fold more potent than the average IC₅₀ for the drug-resistant cell lines of Table 3.

58. The compound of any of claims 46-49, wherein:
E is pyrimidynyl or pyrazolyl;
L³ is —CH₂CH₂—;
Y_{b} is heterocyclyl;
Z¹ is cycloalkyl; and
q is 0, 1 or 2.

59. A compound selected from: 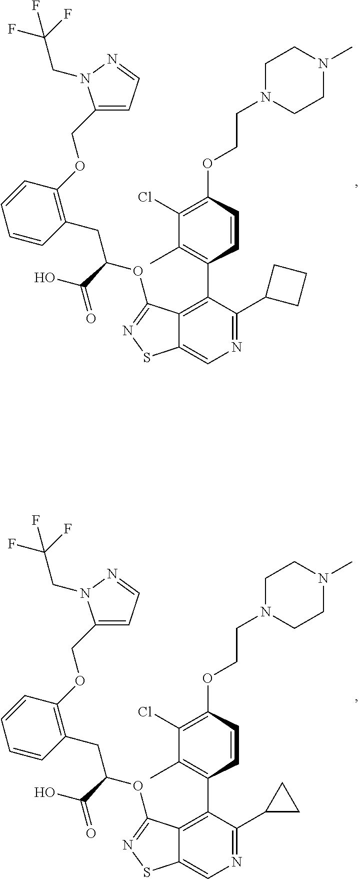 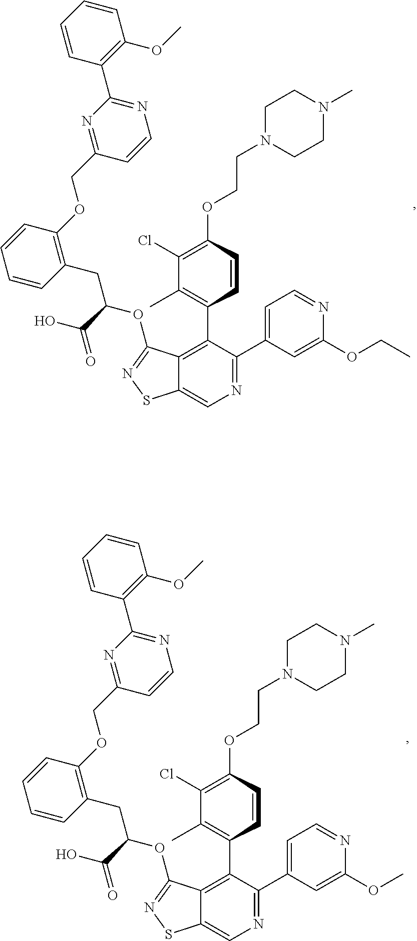 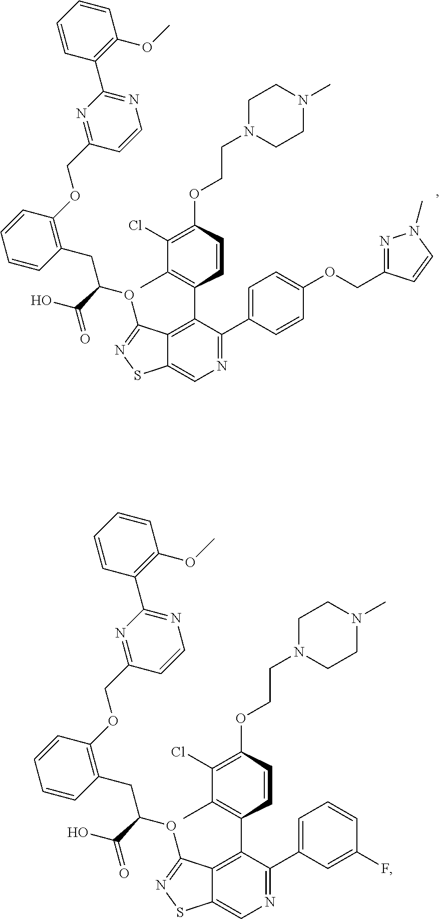 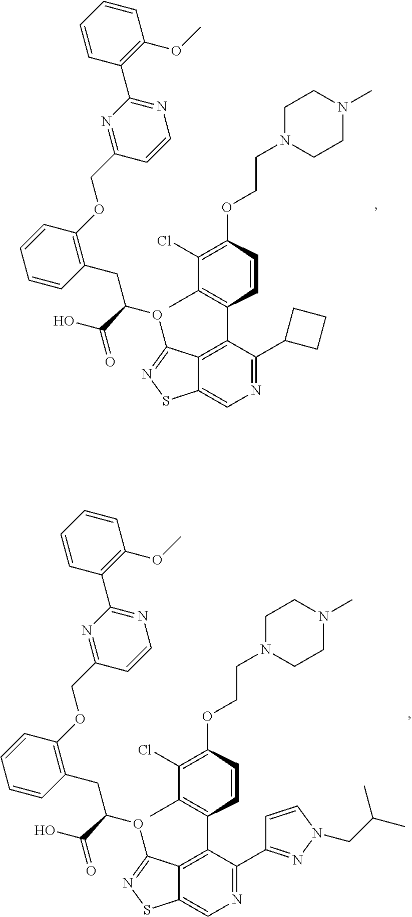 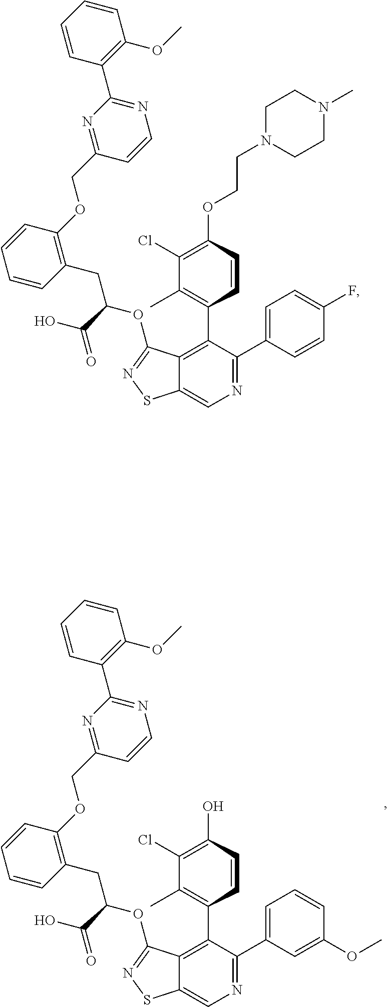 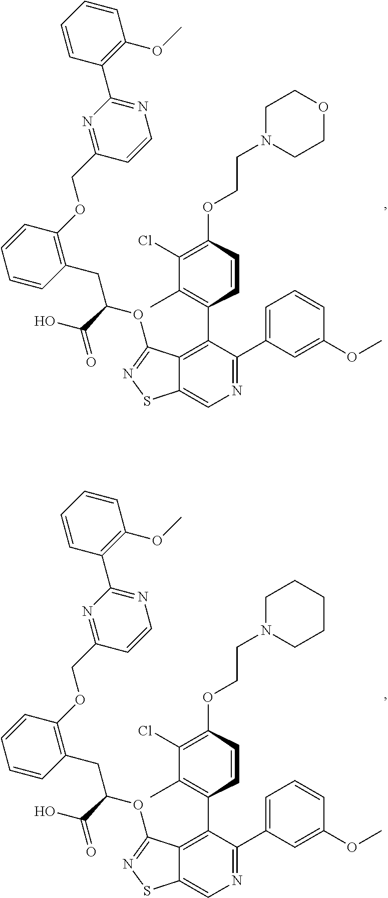 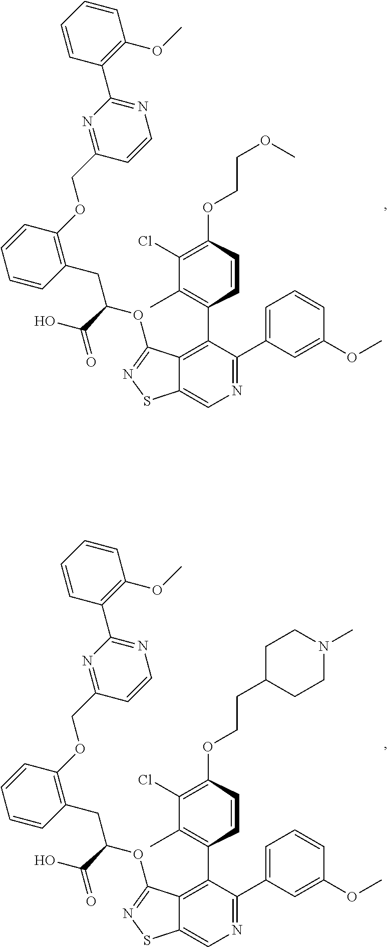 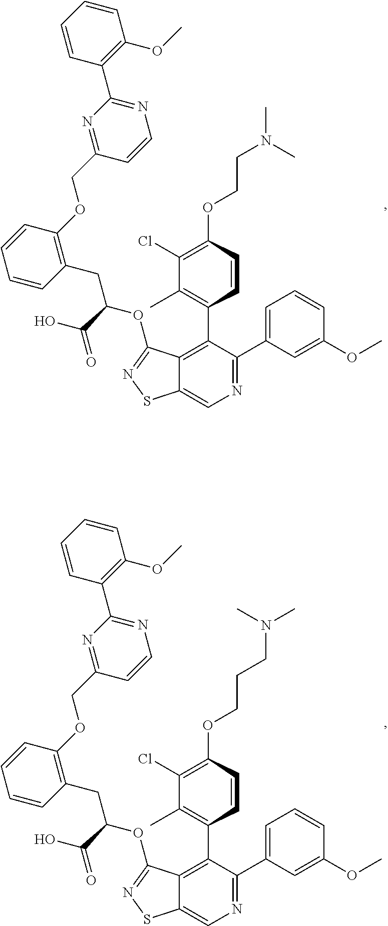 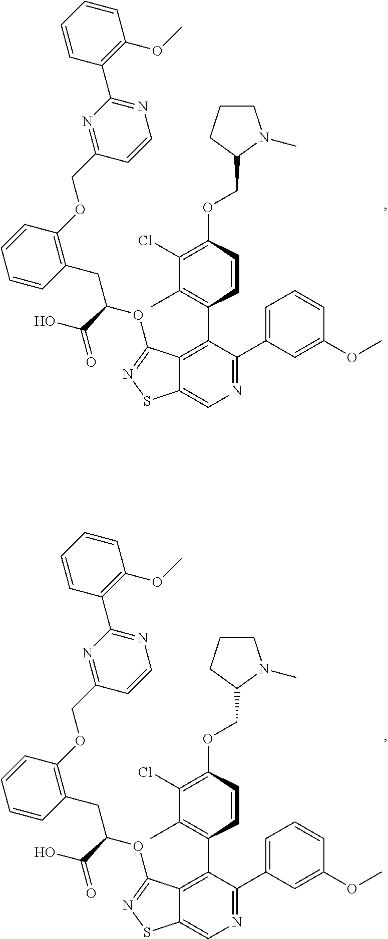 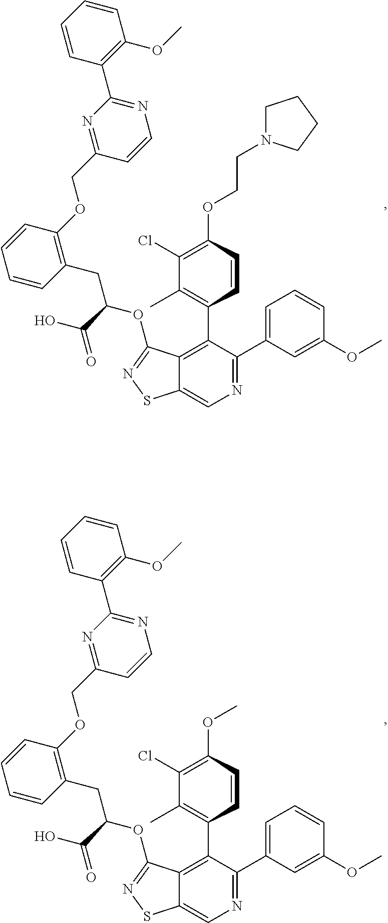 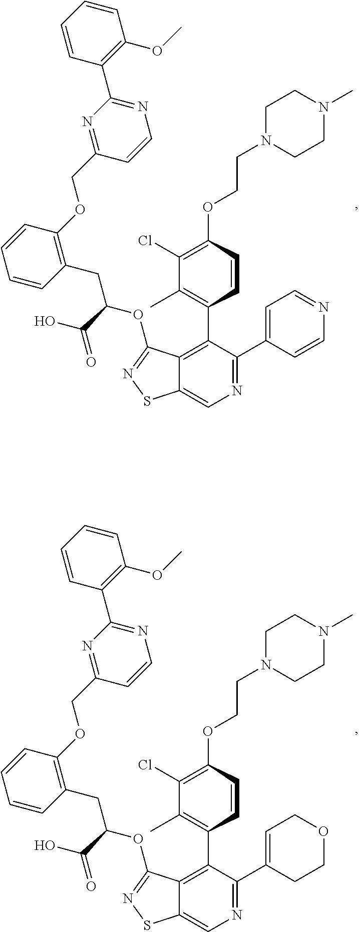 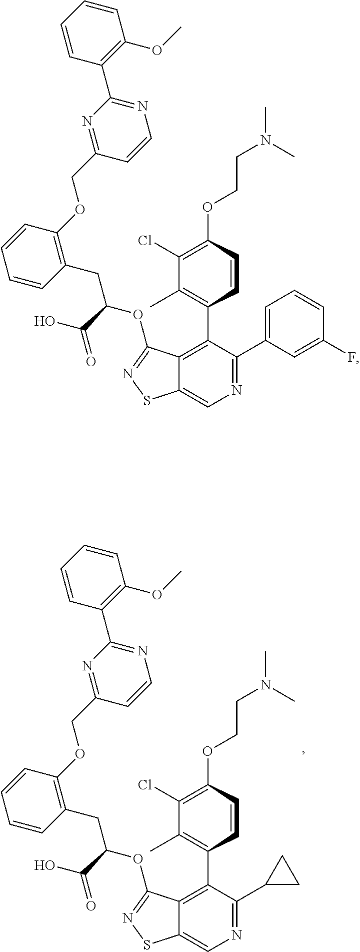 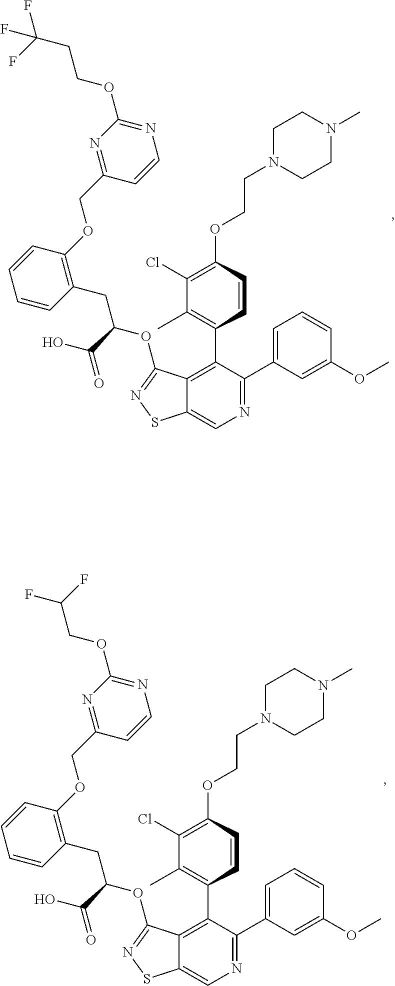 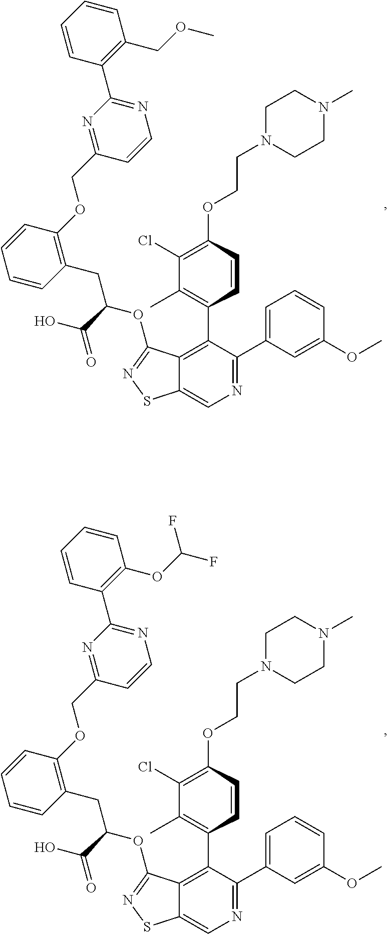 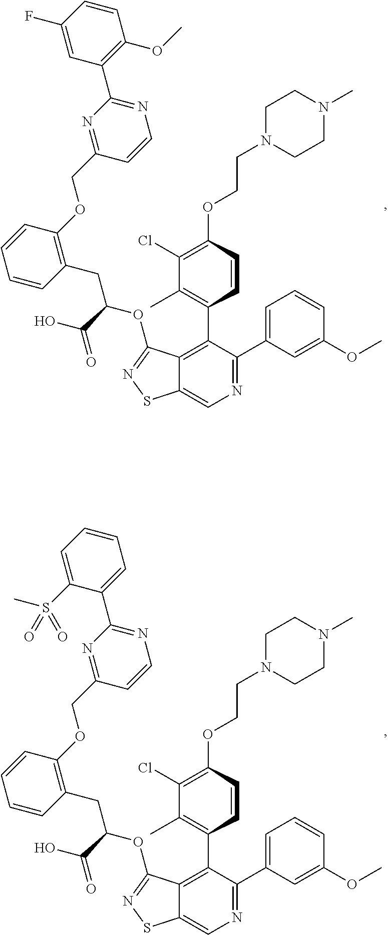 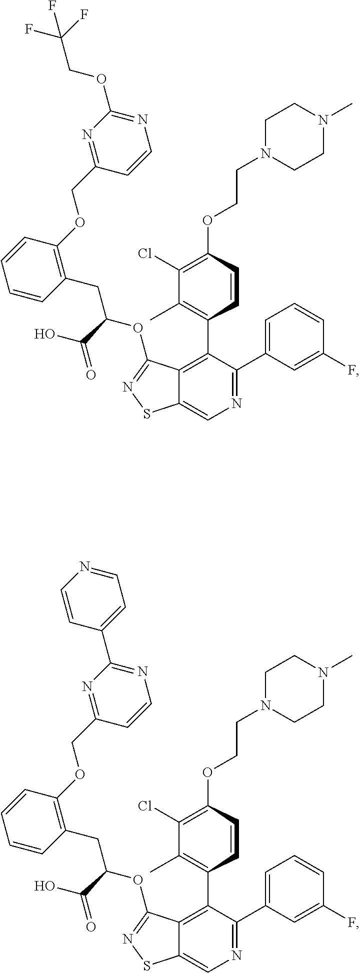 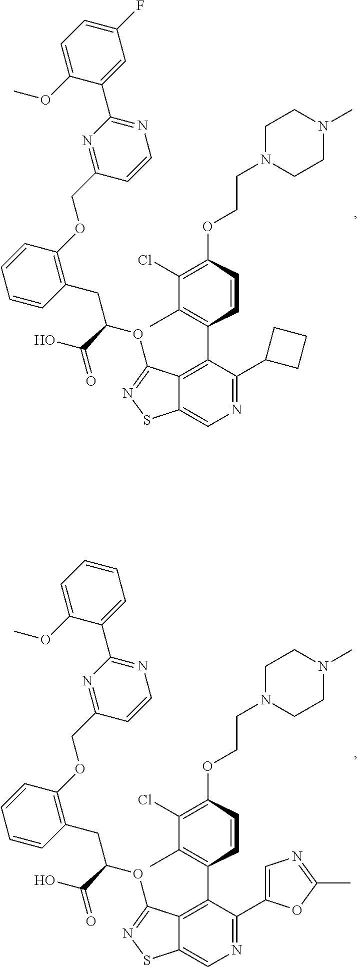 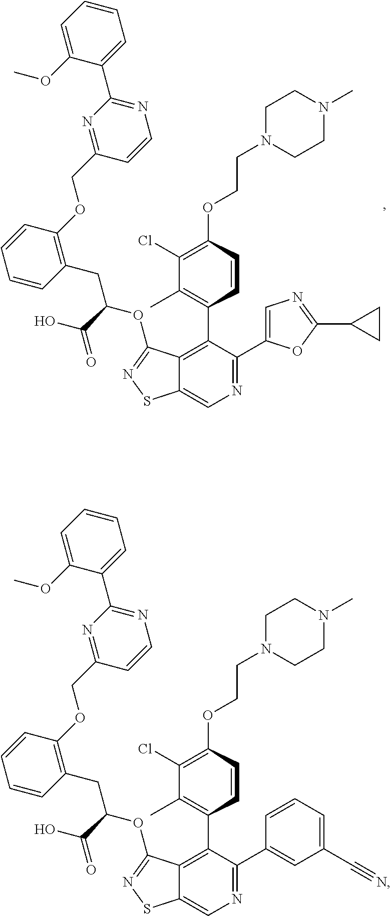 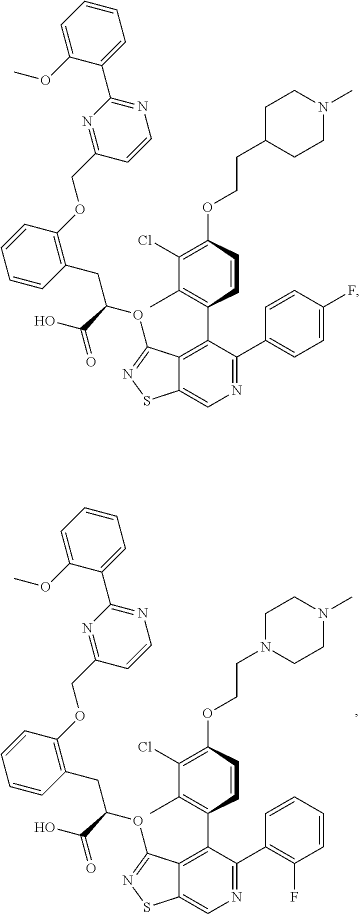 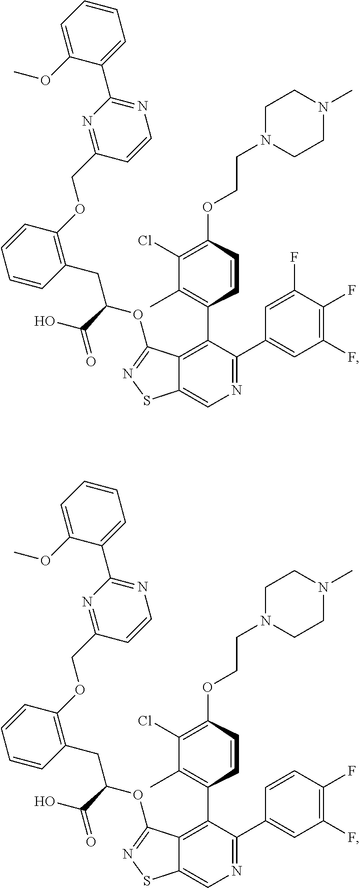 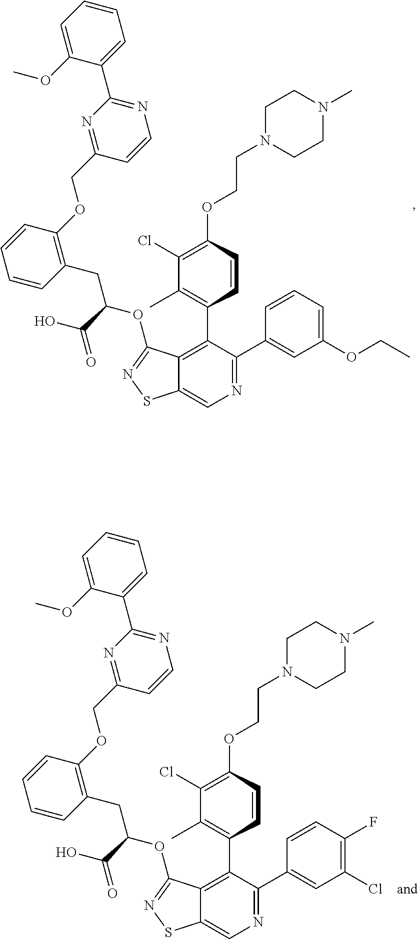 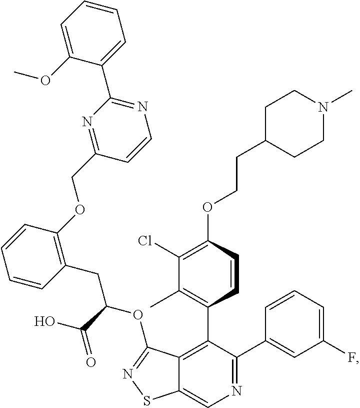
or a pharmaceutically acceptable salt thereof.

60. A compound having the structure of Formula IX: 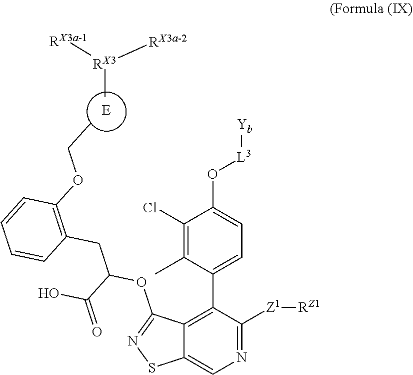
or a pharmaceutically acceptable salt thereof,
wherein:
E is pyrimidinyl, pyrazolyl, pyridinyl or imidazolyl;
L³ is —CH₂— or —CH₂CH₂—;
Y_{b} morpholinyl, piperazinyl, piperindinyl, N(CH₂CH₃)₂ or N(CH₃)₂;
Z¹ is cyclobutyl, benzyl, pyridinyl, pyrazolyl or imidazolyl;
R^{X3} benzyl, pyridinyl, C₁-C₃ alkoxy or C₁-C₄ alkyl;
R^{X3a-1} and R^{X3a-2} is each independently H, C₁-C₃ alkyl, C₁-C₂ haloalkyl, amino, cyano or halogen; and
R^{Z1} is C₁-C₃ alkyl, C₁-C₃ haloalkyl, C₁-C₃ alkoxy or halogen.

61. A compound having the structure of Formula X: 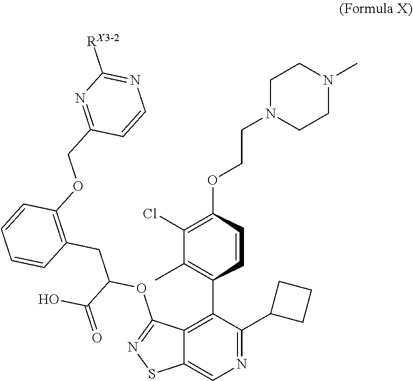
or a pharmaceutically acceptable salt thereof, wherein R^{X3-2} is 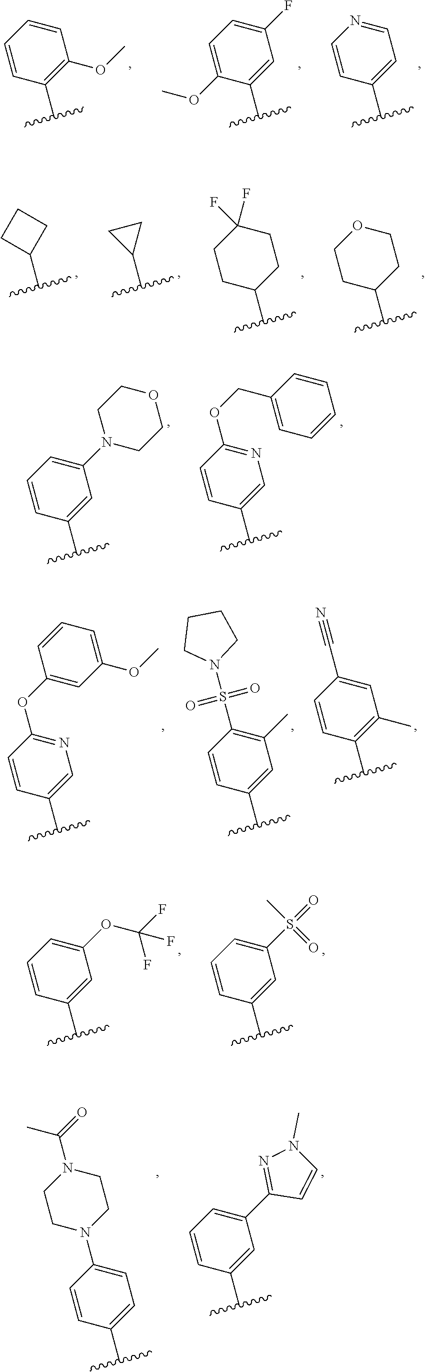 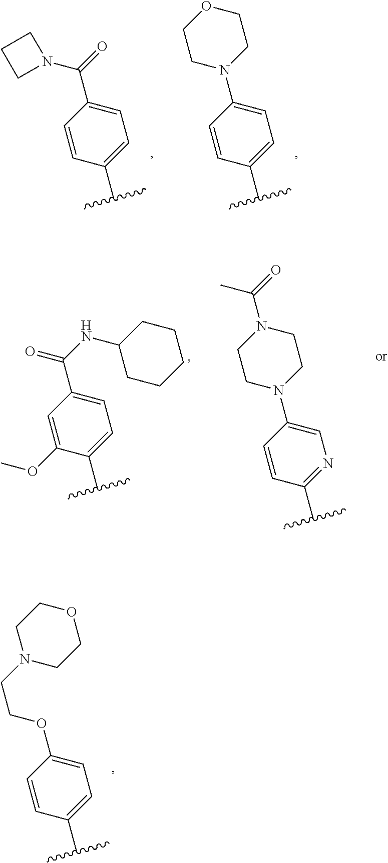 [SMILES: *c1ccc(C(=O)N2CCC2)cc1 ; *c1ccc(N2CCOCC2)cc1 ; *c1ccc(C(=O)NC2CCCCC2)cc1OC ; *c1ccc(N2CCN(C(C)=O)CC2)cn1 ; *c1ccc(OCCN2CCOCC2)cc1]
where [symbol] represents the point of attachment.

62. A compound selected from Table 4.

63. A pharmaceutical composition comprising a compound of any one of claims 1-62 and a pharmaceutically acceptable diluent or excipient.

64. A method of treating a patient afflicted with a disease comprising administering an effective amount of the compound of any one of claims 1-62 or the pharmaceutical composition of claim 63 to the patient so as to thereby treat the disease, wherein the underlying pathology of the disease is mediated by MCL1.

65. The method of claim 64, wherein the disease is a cancer.

66. The method of claim 65, wherein the cancer is selected from a carcinoma, a sarcoma, kidney cancer, epidermis cancer, liver cancer, lung cancer, esophagus cancer, gall bladder cancer, ovary cancer, pancreatic cancer, stomach cancer, cervix cancer, thyroid cancer, nose cancer, head and neck cancer, prostate cancer, skin cancer, breast cancer, familial melanoma, and melanoma.

67. The method of claim 66, wherein the carcinoma is a carcinoma of the endometrium, bladder, breast, or colon; the sarcoma is Kaposi's sarcoma, osteosarcoma, tumor of mesenchymal origin, for example fibrosarcoma or habdomyosarcoma; the lung cancer is adenocarcinoma, small cell lung cancer or non-small cell lung carcinomas; the pancreatic cancer is exocrine pancreatic carcinoma; the skin cancer is squamous cell carcinoma; and the breast cancer is a primary breast tumor, node-negative breast cancer, invasive duct adenocarcinomas of the breast or non-endometrioid breast cancer.

68. The method of claim 65, wherein the cancer is selected from leukemia, acute lymphocytic leukemia, mantle cell lymphoma, chronic lymphocytic leukemia, B-cell lymphoma, diffuse large B cell lymphoma, T-cell lymphoma, multiple myeloma, Hodgkin's lymphoma, non-Hodgkin's lymphoma, hairy cell lymphoma, and Burkett's lymphoma, acute and chronic myelogenous leukemias, myelodysplastic syndrome, and promyelocytic leukemia.

69. The method of claim 65, wherein the cancer is selected from astrocytoma, neuroblastoma, glioma, schwannoma, seminoma, teratocarcinoma, xeroderma pigmentosum, retinoblastoma, keratoctanthoma, and thyroid follicular cancer.

70. The method of claim 65, wherein the cancer is selected from head and neck cancer, sarcoma, melanoma, myeloma, lymphoma, lung cancer, breast cancer, pancreatic cancer, thyroid cancer, colorectal cancer, ovarian cancer and acute myelogenous leukemia.
